SEQUENCE LISTING (14 sequences)

SEQ ID NO: 1 - DNA, 1813 bp, Human
  CDS (31)..(1491)
  mat_peptide (124)..(1491)
  “rBPI”
caggccttgaggttttggcagctctggaggatgagagagaacatggccaggggcccttgcaacgcgccgagatgggtgtccctgatggtgctcgtcgccataggcaccgccgtgacagcggccgtcaaccctggcgtcgtggtcaggatctcccagaagggcctggactacgccagccagcaggggacggccgctctgcagaaggagctgaagaggatcaagattcctgactactcagacagctttaagatcaagcatcttgggaaggggcattatagcttctacagcatggacatccgtgaattccagcttcccagttcccagataagcatggtgcccaatgtgggccttaagttctccatcagcaacgccaatatcaagatcagcgggaaatggaaggcacaaaagagattcttaaaaatgagcggcaattttgacctgagcatagaaggcatgtccatttcggctgatctgaagctgggcagtaaccccacgtcaggcaagcccaccatcacctgctccagctgcagcagccacatcaacagtgtccacgtgcacatctcaaagagcaaagtcgggtggctgatccaactcttccacaaaaaaattgagtctgcgcttcgaaacaagatgaacagccaggtctgcgagaaagtgaccaattctgtatcctccaagctgcaaccttatttccagactctgccagtaatgaccaaaatagattctgtggctggaatcaactatggtctggtggcacctccagcaaccacggctgagaccctggatgtacagatgaagggggagttttacagtgagaaccaccacaatccacctccctttgctccaccagtgatggagtttcccgctgcccatgaccgcatggtatacctgggcctctcagactacttcttcaacacagccgggcttgtataccaagaggctggggtcttgaagatgacccttagagatgacatgattccaaaggagtccaaatttcgactgacaaccaagttctttggaaccttcctacctgaggtggccaagaagtttcccaacatgaagatacagatccatgtctcagcctccaccccgccacacctgtctgtgcagcccaccggccttaccttctaccctgccgtggatgtccaggcctttgccgtcctccccaactcctccctggcttccctcttcctgattggcatgcacacaactggttccatggaggtcagcgccgagtccaacaggcttgttggagagctcaagctggataggctgctcctggaactgaagcactcaaatattggccccttcccggttgaattgctgcaggatatcatgaactacattgtacccattcttgtgctgcccagggttaacgagaaactacagaaaggcttccctctcccgacgccggccagagtccagctctacaacgtagtgcttcagcctcaccagaacttcctgctgttcggtgcagacgttgtctataaatgaaggcaccaggggtgccgggggctgtcagccgcacctgttcctgatgggctgtggggcaccggctgcctttccccagggaatcctctccagatcttaaccaagagccccttgcaaacttcttcgactcagattcagaaatgatctaaacacgaggaaacattattcattggaaaagtgcatggtgtgtattttagggattatgagcttctttcaagggctaaggctgcagagatatttcctccaggaatcgtgtttcaattgtaaccaagaaatttccatttgtgcttcatgaaaaaaaacttctggtttttttcatgtg

SEQ ID NO: 2 - protein, 487 aa, Human
  “rBPI”
MRENMARGPCNAPRWVSLMVLVAIGTAVTAAVNPGVVVRISQKGLDYASQQGTAALQKELKRIKIPDYSDSFKIKHLGKGHYSFYSMDIREFQLPSSQISMVPNVGLKFSISNANIKISGKWKAQKRFLKMSGNFDLSIEGMSISADLKLGSNPTSGKPTITCSSCSSHINSVHVHISKSKVGWLIQLFHKKIESALRNKMNSQVCEKVTNSVSSKLQPYFQTLPVMTKIDSVAGINYGLVAPPATTAETLDVQMKGEFYSENHHNPPPFAPPVMEFPAAHDRMVYLGLSDYFFNTAGLVYQEAGVLKMTLRDDMIPKESKFRLTTKFFGTFLPEVAKKFPNMKIQIHVSASTPPHLSVQPTGLTFYPAVDVQAFAVLPNSSLASLFLIGMHTTGSMEVSAESNRLVGELKLDRLLLELKHSNIGPFPVELLQDIMNYIVPILVLPRVNEKLQKGFPLPTPARVQLYNVVLQPHQNFLLFGADVVYK

SEQ ID NO: 3 - DNA, 24 bp, Human
  BPI-53
actggttccatggaggtcagcgcc

SEQ ID NO: 4 - DNA, 29 bp, Human
  BPI-54
gacagatctctcgagtcatttatagacaa

SEQ ID NO: 5 - DNA, 42 bp, Human
  oligonucleotide from XcmI site to SphI site within BPI gene (encoding residues 348-361) containing the codon TCC for the serine at amino acid position 351
cccaactcctccctggcttccctcttcctgattggcatgcac

SEQ ID NO: 6 - DNA, 42 bp, Human
  Oligonucleotide complementary to SEQ ID NO5
gggttcaggagggaccgaagggagaaggactaaccgtacgtg

SEQ ID NO: 7 - protein, 14 aa, Human
  “wild type” amino acid sequence of residues 348-361 in BPI
PNSSLASLFLIGMH

SEQ ID NO: 8 - DNA, 42 bp, Human
  oligonucleotide from XcmI site to SphI site within the BPI gene (encoding residues 348-361) containing the codon GCC for alanine at amino acid position 351
cccaactccgccctggcttccctcttcctgattggcatgcac

SEQ ID NO: 9 - DNA, 42 bp, Human
  Oligonucleotide complementary to SEQ ID NO8
gggttcaggcgggaccgaagggagaaggactaaccgtacgtg

SEQ ID NO: 10 - protein, 14 aa, Human
  “nonglycosylated” amino acid sequence of residues 348-361 in BPI
PNSALASLFLIGMH

SEQ ID NO: 11 - protein, 456 aa, Human
  bactericidal/permeability-increasing protein (BPI) (Figure 5)
VNPGVVVRISQKGLDYASQQGTAALQKELKRIKIPDYSDSFKIKHLGKGHYSFYSMDIREFQLPSSQISMVPNVGLKFSISNANIKISGKWKAQKRFLKMSGNFDLSIEGMSISADLKLGSNPTSGKPTITCSSCSSHINSVHVHISKSKVGWLIQLFHKKIESALRNKMNSQVCEKVTNSVSSELQPYFQTLPVMTKIDSVAGINYGLVAPPATTAETLDVQMKGEFYSENHHNPPPFAPPVMEFPAAHDRMVYLGLSDYFFNTAGLVYQEAGVLKMTLRDDMIPKESKFRLTTKFFGTFLPEVAKKFPNMKIQIHVSASTPPHLSVQPTGLTFYPAVDVQAFAVLPNSSLASLFLIGMHTTGSMEVSAESNRLVGELKLDRLLLELKHSNIGPFPVELLQDIMNYIVPILVLPRVNEKLQKGFPLPTPARVQLYNVVLQPHQNFLLFGADVVYK

SEQ ID NO: 12 - protein, 456 aa, Human
  lipopolysaccharide binding protein (LBP) (Figure 5)
ANPGLVARITDKGLQYAAQEGLLALQSELLRITLPDFTGDLRIPHVGRGRYEFHSLNIHSCELLHSALRPVPGQGLSLSISDSSIRVQGRWKVRKSFFKLQGSFDVSVKGISISVNLLLGSESSGRPTVTASSCSSDIADVEVDMSGDLGWLLNLFHNQIESKFQKVLESRICEMIQKSVSSDLQPYLQTLPVTTEIDSFADIDYSLVEAPRATAQMLEVMFKGEIFHRNHRSPVTLLAAVMSLPEEHNKMVYFAISDYVFNTASLVYHEEGYLNFSITDDMIPPDSNIRLTTKSFRPFVPRLARLYPNMNLELQGSVPSAPLLNFSPGNLSVDPYMEIDAFVLLPSSSKEPVFRLSVATNVSATLTFNTSKITGFLKPGKVKVELKESKVGLFNAELLEALLNYYILNTFYPKFNDKLAEGFPLPLLKRVQLYDLGLQIHKDFLFLGANVQYMRV

SEQ ID NO: 13 - protein, 476 aa, Human
  phospholipid transfer protein (PLTP) (Figure 5)
EFPGCKIRVTSKALELVKQEGLRFLEQELETITIPDLRGKEGHFYYNISEVKVTELQLTSSELDFQPQQELMLQITNASLGLRFRRQLLYWFFYDGGYINASAEGVSIRTGLELSRDPAGRMKVSNVSCQASVSRMHAAFGGTFKKVYDFLSTFITSGMRFLLNQQICPVLYHAGTVLLNSLLDTVPVRSSVDELVGIDYSLMKDPVASTSNLDMDFRGAFFPLTERNWSLPNRAVEPQLQEEERMVYVAFSEFFFDSAMESYFRAGALQLLLVGDKVPHDLDMLLRATYFGSIVLLSPAVIDSPLKLELRVLAPPRCTIKPSGTTISVTASVTIALVPPDQPEVQLSSMTMDARLSAKMALRGKALRTQLDLRRFRIYSNHSALESLALIPLQAPLKTMLQIGVMPMLNERTWRGVQIPLPEGINFVHEVVTNHAGFLTIGADLHFAKGLREVIEKNRPADVRASTAPTPSTAAV

SEQ ID NO: 14 - protein, 470 aa, Human
  cholesteryl ester transfer protein (CETP) (Figure 5)
HEAGIVCRITKPALLVLNHETAKVIQTAFQRASYPDITGEKAMMLLGQVKYGLHNIQISHLSIASSQVELVEAKSIDVSIQNVSVVFKGTLKYGYTTAWWLGIDQSIDFEIDSAIDLQINTQLTCDSGRVRTDAPDCYLSFHKLLLHLQGEREPGWIKQLFTNFISFTLKLVLKGQICKEINVISNIMADFVQTRAASILSDGDIGVDISLTGDPVITASYLESHHKGHFIYKNVSEDLPLPTFSPTLLGDSRMLYFWFSERVFHSLAKVAFQDGRLMLSLMGDEFKAVLETWGFNTNQEIFQEVVGGFPSQAQVTVHCLKMPKISCQNKGVVVNSSVMVKFLFPRPDQQHSVAYTFEEDIVTTVQASYSKKKLFLSLLDFQITPKTVSNLTESSSESIQSFLQSMITAVGIPEVMSRLEVVFTALMNSKGVSLFDIINPEIITRDGFLLLQMDFGFPEHLLVDFLQSLS

(19) United States
(12) Patent Application Publication
(10) Pub. No.: US 2004/0014153 A1
(43) Pub. Date: Jan. 22, 2004

(54) Bactericidal/permeability-increasing protein: crystallization, x-ray diffraction, three-dimensional structure determination, rational drug design and molecular modeling of related proteins

(76) Inventors: Lesa J. Beamer, Columbia, MO (US); Stephen F. Carroll, Walnut Creek, CA (US); David Eisenberg, Los Angeles, CA (US); Gary Kleiger, Los Angeles, CA (US)

Correspondence Address: Janet M. McNicholas, Ph.D., McAndrews, Held & Malloy, Ltd., 34th Floor, 500 W. Madison, Chicago, IL, 60661, US

(21) Appl. No.: 10/162,743

(22) Filed: Jun. 5, 2002

Related U.S. Application Data
(63) Continuation of application No. 09/872,128, filed on May 31, 2001.
Application No. 09/872,128 is a continuation-in-part of application No. 09/518,598, filed on Mar. 3, 2000.
Application No. 09/518,598 is a continuation of application No. 08/879,565, filed on Jun. 20, 1997, now Pat. No. 6,093,573.
Application No. 09/518,598 is a continuation-in-part of application No. 09/446,415, filed on Jul. 19, 2000.
Application No. 09/446,415 is a 371 of international application No. PCT/US98/13007, filed on Jun. 22, 1998.
Application No. 09/518,598 is a continuation-in-part of application No. 08/879,565, filed on Jun. 20, 1997, now Pat. No. 6,093,573.

(51) Int. Cl. G01N 33/554; G01N 33/569; G06F 19/00; G01N 33/48; G01N 33/50

(52) U.S. Cl. 435/7.32; 702/19

(57) ABSTRACT

The present invention solves the three-dimensional structure of BPI and thereby provides atomic coordinates of BPI from the analysis of x-ray diffraction patterns of sufficiently high resolution for three-dimensional structure determination of the protein, as well as methods for rational drug design, based on using amino acid sequence data and/or x-ray diffraction data provided on computer readable media, as analyzed on a computer system having suitable computer algorithms; and atomic coordinates are provided yielding structural information on related proteins, including the lipid binding and lipid transport protein family that includes BPI, LBP, CETP and PLTP.

This application claims priority as a continuation-in-part of each U.S. application Ser. Nos. 09/518,598, filed Mar. 3, 2000; 09/446,415 which is the national phase of PCT/US98/13007, filed Jun. 22, 1998; 08/879,565, filed Jun. 20, 1997, and of International Application PCT/US98/13007, filed Jun. 22, 1998, which are hereby incorporated by reference in its entirety.

Part of the work performed during development of this invention utilized U.S. Government funds. The U.S. Government has certain rights in this invention.

The present invention generally pertains to the fields of protein crystallization, x-ray diffraction analysis, three-dimensional structural determination, rational drug design and molecular modeling of related proteins. The present invention solves the three-dimensional structure of bactericidal/permeability-increasing protein (BPI) and provides crystallization methods for BPI protein products. A crystallized BPI protein product was physically analyzed by x-ray diffraction techniques. The resulting x-ray diffraction patterns were of sufficiently high resolution to be useful for determining the three-dimensional structure of BPI and have yielded atomic coordinates for BPI. The present invention relates to uses of BPI coordinates for molecular modeling of related proteins and rational drug design (RDD) of mimetics and ligands for BPI and for related proteins. The present invention also relates to atomic coordinates of BPI, or portions thereof, to solve crystal forms of BPI proteins or their fragments, analogs, and variants thereof, or of related proteins, including lipid transfer proteins, or their fragments, analogs and variants.

### BACKGROUND OF THE INVENTION

Bactericidal/permeability-increasing protein (BPI) is a protein isolated from the granules of mammalian polymorphonuclear leukocytes (PMNs or neutrophils), which are blood cells essential in the defense against invading microorganisms. BPI is known to bind the lipopolysaccharide (LPS), a major component of the outer membrane of gram-negative bacteria that stimulates a potent inflammatory response. Human BPI protein has been isolated from PMNs by acid extraction combined with either ion exchange chromatography [Elsbach, J. Biol. Chem., 254:11000 (1979)] or E. coli affinity chromatography [Weiss, et al., Blood, 69:652 (1987)]. BPI obtained in such a manner is referred to herein as natural BPI and has been shown to have potent bactericidal activity against a broad spectrum of gram-negative bacteria. The molecular weight of human BPI is approximately 55,000 daltons (55 kD). The amino acid sequence of the entire human BPI protein and the nucleic acid sequence of DNA encoding the protein have been reported in FIG. 1 of Gray et al., J. Biol. Chem., 264:9505 (1989), incorporated herein by reference. The Gray et al. amino acid sequence is set out in SEQ ID NO: 1 hereto. U.S. Pat. No. 5,198,541, EP0375724 and WO89/10486 (PCT/US88/02700) disclose recombinant genes encoding and methods for expression of BPI proteins, including BPI holoprotein and fragments of BPI.

A proteolytic N-terminal fragment of BPI having a molecular weight of about 25 kD possesses essentially all the anti-bacterial efficacy of the naturally-derived 55 kD human BPI holoprotein. [Ooi et al., J. Bio. Chem., 262: 14891-14894 (1987)]. In contrast to the N-terminal portion, the C-terminal region of the isolated human BPI protein displays only slightly detectable anti-bacterial activity against gram-negative organisms. [Ooi et al., J. Exp. Med., 174:649 (1991).] An N-terminal BPI fragment of approximately 23 kD, referred to as “rBPI₂₃,” has been produced by recombinant means and also retains anti-bacterial activity against gram-negative organisms. [Gazzano-Santoro et al., Infect. Immun. 60:4754-4761 (1992).] An N-terminal analog of BPI, rBPI₂₁, has been produced as described in Horwitz et al., Protein Expression Purification, 8:28-40 (1996).

The bactericidal effect of BPI has been reported to be highly specific to gram-negative species, e.g., in Elsbach and Weiss, Inflammation: Basic Principles and Clinical Correlates, eds. Gallin et al., Chapter 30, Raven Press, Ltd. (1992). This reported target cell specificity was believed to be the result of the strong attraction of BPI for LPS on the outer membrane (or envelope) of gram-negative organisms. Although BPI was commonly thought to be non-toxic for other microorganisms, including yeast and for higher eukaryotic cells, it has recently been discovered that BPI protein products exhibit activity against gram-positive bacteria, mycoplasma, mycobacteria, fungi, protozoa and chlamydia.

The precise mechanism by which BPI kills gram-negative bacteria is not yet completely elucidated, but it is believed that BPI must first bind to the surface of the bacteria through electrostatic and hydrophobic interactions between the cationic BPI protein and negatively charged sites on lipopolysaccharide. Bacterial LPS has been referred to as “endotoxin” because of the potent inflammatory response that it stimulates, i.e., the release of mediators by host inflammatory cells which may ultimately result in irreversible endotoxic shock. BPI binds to lipid A, reported to be the most toxic and most biologically active component of LPS.

In susceptible gram-negative bacteria, BPI binding is thought to disrupt LPS structure, leading to activation of bacterial enzymes that degrade phospholipids and peptidoglycans, altering the permeability of the cell's outer membrane, and initiating events that ultimately lead to cell death. [Elsbach and Weiss (1992), supra]. BPI has been proposed to act in two stages. The first stage proposed is a sublethal one that is characterized by immediate growth arrest, permeabilization of the outer membrane and selective activation of bacterial enzymes that hydrolyze phospholipids and peptidoglycans. Bacteria at this stage could be rescued by growth in serum albumin supplemented media [Mannion et al., J. Clin. Invest., 85:853-860 (1990)]. The second stage, defined by growth inhibition that cannot be reversed by serum albumin, is proposed to occur after prolonged exposure of the bacteria to BPI and characterized by extensive physiologic and structural changes, including apparent damage to the inner cytoplasmic membrane.

Initial binding of BPI to LPS leads to organizational changes that probably result from binding to the anionic groups of LPS, which normally stabilize the outer membrane through binding of Mg⁺⁺ and Ca⁺⁺. Attachment of BPI to the outer membrane of gram-negative bacteria produces rapid permeabilization of the outer membrane to hydrophobic agents such as actinomycin D. Binding of BPI and subsequent gram-negative bacterial killing depends, at least in part, upon the LPS polysaccharide chain length, with long O-chain bearing, “smooth” organisms being more resistant to BPI bactericidal effects than short O-chain bearing, “rough” organisms [Weiss et al., J. Clin. Invest. 65: 619-628 (1980)]. This permeabilization of the gram-negative outer envelope is reversible upon dissociation of the BPI, a process requiring high concentrations of divalent cations and synthesis of new LPS [Weiss et al., J. Immunol. 132: 3109-3115 (1984)]. Loss of gram-negative bacterial viability, however, is not reversed by processes which restore the envelope integrity, suggesting that the bactericidal action is mediated by additional lesions induced in the target organism and which may be situated at the cytoplasmic membrane (Mannion et al., J. Clin. Invest. 86: 631-641 (1990)). Specific investigation of this possibility has shown that on a molar basis BPI is at least as inhibitory of cytoplasmic membrane vesicle function as polymyxin B but the exact mechanism as well as the relevance of such vesicles to studies of intact organisms was not elucidated (In't Veld, et al., Infection and Immunity 56: 1203-1208 (1988)).

BPI is a member of a gene/protein family of lipopolysaccharide binding and lipid transfer proteins whose other currently known members include lipopolysaccharide binding protein (LBP), cholesteryl ester transfer protein (CETP) and phospholipid transfer protein (PLTP).

BPI protein products (which include naturally and recombinantly produced BPI protein; natural, synthetic, and recombinant biologically active polypeptide fragments of BPI protein; biologically active polypeptide variants of BPI protein or fragments thereof, including hybrid fusion proteins and dimers; biologically active polypeptide analogs of BPI protein or fragments or variants thereof, including cysteine-substituted analogs; and BPI-derived peptides) have been demonstrated to have a variety of beneficial activities. BPI protein products are known to be bactericidal for gram-negative bacteria, as described in U.S. Pat. Nos. 5,198,541 and 5,523,288, both of which are incorporated herein by reference. BPI protein products are also known to enhance the effectiveness of antibiotic therapy in gram-negative bacterial infections, as described in U.S. Pat. No. 5,523,288 and corresponding International Publication No. WO 95/08344 (PCT/US94/11225), which are incorporated herein by reference. BPI protein products are also known to be bactericidal for gram-positive bacteria and mycoplasma, and to enhance the effectiveness of antibiotics in gram-positive bacterial infections, as described in U.S. Pat. No. 5,578,572 and corresponding International Publication No. WO 95/19180 (PCT/US95/00656), which are incorporated herein by reference. BPI protein products are further known to exhibit anti-fungal activity, and to enhance the activity of other anti-fungal agents, as described in U.S. Pat. No. 5,627,153 and corresponding International Publication No. WO 95/19179 (PCT/US95/00498), and further as described for anti-fungal peptides in U.S. Pat. Nos. 6,156,730 and 5,858,974, which is in turn a continuation-in-part of U.S. application Ser. No. 08/504,841 filed Jul. 20, 1995 and corresponding International Publication Nos. WO 96/08509 (PCT/US95/09262) and WO 97/04008 (PCT/US96/03845), all of which are incorporated herein by reference. BPI protein products are further known to exhibit anti-protozoan activity, as described in U.S. Pat. Nos. 5,646,114 and 6,013,629 and corresponding International Publication No. WO 96/01647 (PCT/US95/08624), all of which are incorporated herein by reference. BPI protein products are known to exhibit anti-chlamydial activity, as described in U.S. Pat. Nos. 5,888,973 and 6,162,788, and corresponding International Publication No. WO 98/06415 (PCT/US97/13810), all of which are incorporated herein by reference. Finally, BPI protein products are known to exhibit anti-mycobacterial activity, as described in U.S. Pat. No. 6,214,289 which is in turn a continuation of U.S. application Ser. No. 08/285,803 filed Aug. 14, 1994, which is in turn a continuation-in-part of U.S. application Ser. No. 08/031,145 filed Mar. 12, 1993 and corresponding International Publication No. WO94/20129 (PCT/US94/02463), all of which are incorporated herein by reference.

The effects of BPI protein products in humans with endotoxin in circulation, including effects on TNF, IL-6 and endotoxin are described in U.S. Pat. Nos. 5,643,875, 5,753,620, 5,952,302 and 6,191,112 and corresponding International Publication No. WO 95/19784 (PCT/US95/01151), all of which are incorporated herein by reference.

BPI protein products are also known to be useful for treatment of specific disease conditions, such as meningococcemia in humans (as described in U.S. Pat. Nos. 5,888,977 and 5,990,086, and corresponding International Publication No. WO 97/42966 (PCT/US97/08016), which are incorporated herein by reference), hemorrhagic trauma in humans, (as described in U.S. Pat. No. 5,945,399, a continuation-in-part of U.S. application Ser. No. 08/652,292 filed May 23, 1996, now U.S. Pat. No. 5,756,464, and corresponding International Publication No. WO 97/44056 (PCT/US97/08941), all of which are incorporated herein by reference), burn injury (as described in U.S. Pat. No. 5,494,896 and corresponding International Publication No. WO 96/30037 (PCT/US96/02349), both of which are incorporated herein by reference), ischemia/reperfusion injury (as described in U.S. Pat. Nos. 5,578,568 and 6,017,881, incorporated herein by reference), and liver resection (as described in co-pending U.S. application Ser. No. 09/689,097 filed Oct. 12, 2000 which is a continuation of U.S. application Ser. No. 09/466,412 filed Dec. 17, 1999 which is a continuation of U.S. application Ser. No. 08/582,230 filed Mar. 16, 1998 which is a continued prosecution application of the same serial no. filed Jan. 3, 1996, which is in turn a continuation of U.S. application Ser. No. 08/318,357 filed Oct. 5, 1994, which is in turn a continuation-in-part of U.S. application Ser. No. 08/132,510 filed Oct. 5, 1993, and corresponding International Publication No. WO 95/10297 (PCT/US94/11404), all of which are incorporated herein by reference).

BPI protein products are also known to neutralize the anti-coagulant activity of exogenous heparin, as described in U.S. Pat. No. 5,348,942, incorporated herein by reference, as well as to be useful for treating chronic inflammatory diseases such as rheumatoid and reactive arthritis, as described in U.S. Pat. No. 5,639,727, incorporated herein by reference, and for inhibiting angiogenesis and for treating angiogenesis-associated disorders including malignant tumors, ocular retinopathy and endometriosis, as described in U.S. Pat. Nos. 5,807,818, 5,837,678 and 5,854,214, and corresponding International Publication No. WO 94/20128 (PCT/US94/02401), all of which are incorporated herein by reference.

BPI protein products are also known for use in antithrombotic methods, as described in U.S. Pat. Nos. 5,741,779, 5,935,930 and 6,107,280, and corresponding International Publication No. WO97/42967 (PCT/US97/08017), which are incorporated herein by reference.

U.S. Pat. Nos. 5,420,019, 5,827,816 and 5,674,834 and corresponding International Publication No. WO94/18323 (PCT/US94/01235), all of which are incorporated herein by reference, disclose that the replacement of the cysteine residue at amino acid position 132 or 135 with another amino acid renders the resulting BPI polypeptide resistant to dimerization and cysteine adduct formation. It also discloses that terminating the N-terminal BPI fragment at BPI amino acid position 193 resulted in an expression product with reduced carboxy-terminal heterogeneity.

Because of the multiplicity of valuable activities and uses of BPI protein products as exemplified above, a need continues to exist for new products with structures based on or mimicking a BPI protein product and having one or more of the activities and/or uses of BPI protein products, including use as anti-infective products, including antimicrobial agents (e.g., gram-negative bacteria [U.S. Pat. Nos. 5,198,541 and 5,523,288; WO95/08344 (PCT/US94/11225)] and gram-positive bacteria [U.S. Pat. Nos. 5,578,572, 5,783,561 and 6,054,431; WO95/19180 (PCT/US95/00656)], fungi [U.S. Pat. No. 5,627,153; WO95/19179 (PCT/US95/00498)], mycobacteria [U.S. Pat. No. 6,214,789, EP0690721; WO94/20129 (PCT/US94/02463)] and chlamydia [U.S. Pat. No. 6,162,788, WO96/01647 (PCT/US95/08624)] and endotoxin binding/neutralizing agents [WO95/019784 (PCT/US95/01151)], and as heparin binding/neutralizing products [U.S. Pat. Nos. 5,348,942 and 5,639,727; WO94/20128 (PCT/US94/02401)], including for the neutralization of exogenously administered heparin, inhibition of angiogenesis (normal or pathological) for the treatment of chronic inflammatory disease states, anticoagulant and thrombolytic agents for the treatment of thrombotic disorders [WO97/42976 (PCT/US97/08017)], for inhibiting H⁺/K⁺ ATPase activity [WO01/03724 (PCT/US00/09125)] for modulation of pericyte proliferation [U.S. application Ser. No. 60/250,542], for treating conditions associated with corneal injury [WO97/17990 (PCT/US96/18632)], for treating conditions associated with corneal transplantation [U.S. Pat. No. 5,686,414; WO97/17989 (PCT/US96/18416)], for use in ANCA-positive humans [U.S. application Ser. Nos. 08/742,985 and 09/255,245], for use in cystic fibrosis patients [WO98/19694 (PCT/US97/19850)], for treating chronic cardiac disease [WO00/43028 (PCT/US00/01515)], for treating BPI-deficient humans [U.S. Pat. No. 6,153,584; WO00/59531 (PCT/US00/08864)], and for use in humans with otitis media with effusion [WO00/71149 (PCT/US00/14496)]. All of the above-listed references regarding biological or functional activities of BPI, as well as therapeutic and diagnostic uses of BPI, are hereby incorporated by reference. One avenue of investigation toward solving the problem of new products based on BPI and fulfilling this need is the determination of the crystal structure of a BPI protein product.

### SUMMARY OF THE INVENTION

The present invention solves the above problem and fulfills the need for designing and making new and useful products based on BPI. It is an object of this invention to solve the three-dimensional structure of BPI and thereby provide the atomic coordinates (i.e., structure coordinates) of BPI from the analysis of x-ray diffraction patterns of sufficiently high resolution to be useful for determining the three-dimensional protein structure.

It is an object of this invention to provide methods of expressing, purifying and crystallizing bactericidal/permeability-increasing protein (BPI) products, and thereby provide crystallized BPI proteins.

It is an object of this invention to provide the use of the structure coordinates of a BPI crystal to allow the design of compounds for mimicking a BPI protein product to reveal the atomic details of ligand binding sites of BPI (e.g. lipid-like or heparin-like molecules).

It is an object of this invention to provide use of the structure coordinates of a BPI crystal as described herein to solve the crystal structure of a crystal of a different BPI protein or fragment, analog or variant thereof, or a crystal of a related protein, including a BPI-related lipid transfer protein or a fragment, analog or variant thereof.

It is an object of this invention to provide mutants of BPI or fragments, analogs, or variants thereof characterized by one or more different properties as compared with wild-type BPI. These properties include altered surface charge, altered lipid binding pockets, altered specificity or higher activity. BPI mutants are useful to identify those amino acids that are most important for the lipid and heparin binding activity and other biological activities of BPI. This information, in turn, allows the design of new structures with one or more different properties based on BPI.

It is an object of this invention to provide the use of the structure coordinates and atomic details of BPI as described herein or its fragments, analogs or variants (including mutants or co-complexes) or of a BPI-related lipid transfer protein or its fragments, analogs or variants (including mutants or co-complexes) to design, evaluate computationally, synthesize and use new structures based on BPI with desirable properties of BPI, such as physical and pharmacological properties.

X-ray diffraction patterns of a related protein can be analyzed directly to provide the three-dimensional structure (if of sufficiently high resolution), however, the atomic coordinates for the crystallized BPI, as provided herein, can be used for structure determination. The x-ray diffraction patterns obtained by methods of the present invention, and provided on computer readable media, are used to provide electron density maps. The amino acid sequence is also useful for three-dimensional structure determination. The data is then used in combination with phase determination (e.g. using multiple isomorphous replacement (MIR) molecular replacement techniques) to generate electron density maps of BPI, using a suitable computer system. The electron density maps, provided by analysis of either the x-ray diffraction patterns or working backwards from the atomic coordinates, provided herein, are then fitted using suitable computer algorithms to generate secondary, tertiary and/or quaternary structures and/or domains of BPI, which structures and/or domains are then used to provide an overall three-dimensional structure, as well as binding and/or active sites of BPI.

It is also an object of this invention to specifically provide for the use of three-dimensional modeling of BPI and other members of the BPI protein family using the coordinates from the x-ray diffraction patterns. The coordinates and amino acid sequences are entered into one or more computer programs for molecular modeling. Such molecular modeling programs generate atomic coordinates that reflect the secondary, tertiary and/or quaternary structures of the protein which contribute to its overall three-dimensional structure and provide information related to binding and/or active sites of the protein.

It is a further object of this invention to specifically provide for the use of similar molecular modeling for rational drug design (RDD) of mimetics and ligands of BPI and other members of the BPI protein family. The drug design paradigm uses computer modeling programs to determine potential mimetics and ligands which are expected to interact with sites on the protein. The potential mimetics or ligands are then screened for activity and/or binding. For BPI-related mimetics or ligands, screening methods can be selected from assays for at least one biological activity of BPI, e.g., anti-microbial, LPS-binding/neutralizing, heparin binding/neutralizing, and/or anti-thrombotic activities, according to known method steps. Similarly for LBP-, CETP- or PLTP-related mimetics or ligands, such screening methods can be selected from assays for at least one biological activity of LBP, CETP or PLTP, according to known method steps. The resulting mimetics or ligands are then provided by methods of the present invention and are useful for treating, inhibiting or preventing BPI-modulated diseases (or LBP-, CETP- and PLTP-modulated) in animals, including humans.

Thus, as described herein, the present invention provides use of atomic coordinates of a BPI protein, or fragment, analog or variant thereof, to model a BPI protein or a related protein, including a BPI-related lipid transfer protein, such as LBP, CETP or PLTP, or fragment, analog or variant thereof.

The present invention also provides use of atomic coordinates of a BPI protein wherein the BPI protein comprises a binding site characterized by amino acid residues of at least one binding pocket as defined in Table 3, and/or wherein the BPI protein comprises a binding site characterized by at least one amino acid sequence, or variant of the sequence, selected from positions about 17 to about 45, positions about 65 to about 99 or positions about 142 to about 169 of BPI, or alternatively, a binding site characterized by at least one amino acid sequence, or variant of the sequence, selected from positions about 36 to about 54, positions about 84 to about 109 or positions about 142 to about 164 of BPI.

The present invention provides use of atomic coordinates of a BPI protein to computationally design a chemical compound for mimicking a BPI protein, or fragment, analog or variant thereof, or a BPI-related lipid transfer protein, or fragment, analog or variant thereof, including, for example, lipopolysaccharide-binding protein (LBP), cholesteryl ester transferase protein (CETP) or phospholipid transfer protein (PLTP), or fragment, analog, or variant thereof.

The present invention also provides use of atomic coordinates of BPI protein to design a chemical compound capable of associating with a BPI-related lipid binding protein, or fragment, analog or variant thereof, including, for example, bactericidal/permeability-increasing protein (BPI), lipopolysaccharide-binding protein (LBP), cholesteryl ester transferase protein (CETP) or phospholipid transfer protein (PLTP), or fragment, analog or variant thereof.

The present invention provides use of atomic coordinates of a BPI protein to design a model of ligands in an active site of a lipid binding protein, including, for example, BPI protein, lipopolysaccharide-binding protein (LBP), cholesteryl ester transferase protein (CETP) or phospholipid transfer protein (PLTP), or fragment, analog or variant thereof.

The present invention provides use of atomic coordinates of a bactericidal/permeability-increasing (“BPI”) protein, to design compounds with at least one activity of antibacterial, antifungal, antimycobacterial, antichlamydial, antiprotozoan, heparin-binding, endotoxin-binding, heparin-neutralizing, endotoxin-neutralizing, inhibition of tumor and endothelial cell proliferation, inhibition of angiogenesis, anti-inflammatory, anticoagulant or antithrombolytic, enhancement of pericyte cell proliferation, enhancement of antibiotic activity or susceptibility, or inhibition of H⁺/K⁺ ATPase activity. The coordinates disclosed herein are suitable for all of the aforementioned uses of atomic coordinates.

The present invention provides a method of three-dimensional modeling of a BPI protein or BPI-related lipid transfer protein comprising the steps of: (a) providing three-dimensional atomic coordinates derived from X-ray diffraction measurements of a BPI protein in a computer readable format; (b) inputting the data from step (a) into a computer with appropriate software programs; and (c) generating a three-dimensional structural representation of the BPI protein or BPI-related lipid transfer protein suitable for visualization and further computational manipulation; particularly wherein the BPI protein comprises a binding site characterized by amino acid residues of at least one binding pocket as defined in Table 3, or wherein the BPI protein comprises a binding site characterized by at least one amino acid sequence, or variant of the sequence, selected from positions about 17 to about 45, positions about 65 to about 99 or positions about 142 to about 169 of BPI or wherein the BPI protein comprises a binding site characterized by amino acid residues of at least one binding pocket as defined in Table 3 and a binding site characterized by at least one amino acid sequence, or variant of the sequence, selected from positions about 36 to about 54, positions about 84 to about 109 or positions about 142 to about 164 of BPI, or alternatively from about positions 36 to about 54, from about positions 84 to about 109, or about positions 142 to about 164.

The present invention provides a method for providing an atomic model of a BPI protein, BPI-related lipid binding protein, or fragment, analog or variant thereof, comprising (a) providing a computer readable medium having stored, thereon atomic coordinate/x-ray diffraction data of a BPI protein, or fragment, analog or variant thereof, in crystalline form, the data sufficient to model the three-dimensional structure of the BPI protein, or fragment, analog or variant thereof; (b) analyzing, on a computer using at least one subroutine executed in said computer, atomic coordinate/x-ray diffraction data from (a) to provide atomic coordinate data output defining an atomic model of said BPI protein, BPI-related binding lipid protein or fragment, analog or variant thereof, said analyzing utilizing at least one computing algorithm selected from the group consisting of data processing and reduction, auto-indexing, intensity scaling, intensity merging, amplitude conversion, truncation, molecular replacement, molecular alignment, molecular refinement, electron density map calculation, electron density modification, electron map visualization, model building, rigid body refinement, positional refinement; and (c) obtaining atomic coordinate data defining the three-dimensional structure of at least one of said BPI protein, BPI-related lipid binding protein, or fragment, analog or variant thereof; particularly wherein said computer readable medium further has stored thereon data corresponding to a nucleic acid sequence or an amino acid sequence data comprising at least one structural domain or functional domain of a BPI, LBP, CETP or PLTP corresponding to at least one BPI or mutant primary sequence or fragment, analog or variant thereof; and wherein said analyzing step further comprises analyzing said sequence data.

The present invention provides a computer-based system for providing atomic model data of the three-dimensional structure of a BPI protein, BPI-related lipid binding protein or fragment, analog or variant thereof, comprising the following elements: (a) at least one computer readable medium (CRM) having stored thereon atomic coordinate/x-ray diffraction data of a BPI protein, or fragment, analog or variant thereof; (b) at least one computing subroutine that, when executed in a computer, causes the computer to analyze atomic coordinate/x-ray diffraction data from (a) to provide atomic coordinate data output defining an atomic model of a BPI protein, BPI-related lipid binding protein or fragment, analog or variant thereof, said analyzing utilizing at least one computing subroutine selected from the group consisting of data processing and reduction, auto-indexing, intensity scaling, intensity merging, amplitude conversion, truncation, molecular replacement, molecular alignment, molecular refinement, electron density map calculation, electron density modification, electron map visualization, model building, rigid body refinement, positional refinement; and (c) retrieval means for obtaining atomic coordinate output data substantially defining the three-dimensional structure of said BPI protein, BPI-related lipid binding protein or fragment, analog or variant thereof.

The present invention provides a method for providing a computer atomic model of a ligand of a BPI protein, BPI-related lipid binding protein, or fragment, analog or variant thereof, comprising: (a) providing a computer readable medium (CRM) having stored thereon atomic coordinate data of a BPI protein, or fragment, analog, or variant thereof; (b) providing a CRM having stored thereon atomic coordinate data sufficient to generate atomic models of potential ligands of said BPI protein, BPI-related lipid binding protein, or fragment, analog, or variant thereof; (c) analyzing on a computer, using at least one subroutine executed in said computer, the atomic coordinate data from (a) and ligand data from (b), to determine binding sites of BPI protein, BPI-related lipid binding protein, or fragment, analog, or variant thereof, and to provide atomic coordinate data defining an atomic model of at least one ligand of said BPI protein, BPI-related lipid binding protein, or fragment, analog or variant thereof, said analyzing utilizing computing subroutines selected from the group consisting of data processing and reduction, auto-indexing, intensity scaling, intensity merging, amplitude conversion, truncation, molecular replacement, molecular alignment, molecular refinement, electron density map calculation, electron density modification, electron map visualization, model building, rigid body refinement, positional refinement; and (d) obtaining atomic coordinate model output data defining the three-dimensional structure of said at least one ligand of said BPI protein, BPI-related lipid binding protein, or fragment, analog, or variant thereof.

The present invention provides a computer-based system for providing an atomic model of at least one ligand of a BPI protein, BPI-related lipid binding protein, or fragment, analog or variant thereof, comprising the following elements: (a) a computer readable medium (CRM) having stored thereon atomic coordinate data of a BPI protein, fragment, analog or variant thereof; (b) a CRM having stored thereon atomic coordinate data sufficient to generate atomic models of potential ligands of a BPI protein, BPI-related lipid binding protein, or fragment, analog or variant thereof; (c) at least one computing subroutine for analyzing on a computer, the atomic coordinate data from (a) and (b), to determine binding sites of BPI protein, BPI-related lipid binding protein, or fragment, analog, or variant thereof, and to provide data output defining an atomic model of at least one potential ligand of BPI protein, BPI-related lipid binding protein, or fragment, analog, or variant thereof, said analyzing utilizing at least one computing subroutine selected from the group consisting of data processing and reduction, auto-indexing, intensity scaling, intensity merging, amplitude conversion, truncation, molecular replacement, molecular alignment, molecular refinement, electron density map calculation, electron density modification, electron map visualization, model building, rigid body refinement, positional refinement; and (d) retrieval means for obtaining atomic coordinate data of said at least one ligand of a BPI protein, BPI-related lipid binding protein, or fragment, analog or variant thereof.

Other objects of the invention will be apparent to one of ordinary skill in the art from the following detailed description and examples relating to the present invention.

### BRIEF DESCRIPTION OF THE FIGURES

FIG. 1(A) A ribbon diagram of residues 1-456 of BPI illustrating its boomerang shape. The NH₂-terminal domain is shown; the COOH-terminal domain and the two phosphatidylcholine molecules are shown. The linker is also shown, and the disulfide bond is shown as a ball-and-stick model. (B) View after rotating (A) 70° about the long axis of the molecule. Figure produced with MOLSCRIPT [P. Krauliz, J. Appl. Cryst., 24:926 (1991)] and RASTER3D [E. A. Merrit and M. E. P. Murphy, Acta Crystallogr., D50:889 (1994); D. J. Bacon and W. F. Anderson, J. Mo. Graphics, 6:219 (1988)].

FIG. 2(A) Schematic drawing of the novel BPI domain fold, shown in same orientation as the NH₂-terminal domain in FIG. 1B (B) Superposition of the NH₂- and COOH-terminal domains of BPI showing the overall topological similarity. Residues 1 to 230 and 250 to 456 are shown. The NH₂-terminal domain is in the same orientation as FIG. 1A.

FIG. 3 Electron density of the final 2.8 Å MIR map contoured at 1.0σ and superimposed on the refined model. The area shown is in the lipid binding pocket of the NH₂-terminal domain of BPI. The phosphatidylcholine and the surrounding protein atoms are shown.

FIG. 4(A) The covalent structure of phosphatidylcholine and the lipid A region of LPS from E. coli and S. typhimurium. Phosphate groups are indicated by P. Adapted with changes from [C. R. H. Raetz, Annu. Rev. Biochem, 59:129 (1990)]. (B) Slice through the interior of BPI showing the lipid binding pocket in the NH₂-terminal domain. The solvent accessible surface of the protein was calculated without lipid present, the interior of the protein and the phosphatidylcholine are shown. Protein residues are shown as ball-and-stick. Figure produced with MSP [M. L. Connolly, Science, 221:709 (1983); M. L. Connolly, J. Am. Chem. Soc., 107:1118 (1985)].

FIGS. 5(A) and 5(B) The amino acid sequences of human BPI, LBP, PLTP, and CETP. The alignment was performed with CLUSTAL [D. G. Higgins and P. M. Sharp, Gene, 73:237 (1989)] using all eleven known protein sequences from mammals [R. R. Schuman, et al., Science, 249:1429 (1990); D. Drayna et al., Nature, 327:632 (1987); R. Day et al., J. Biol. Chem., 269:9388 (1994); S. R. Leong and T. Camerato, Nucleic Acids Res., 18:3052 (1990); M. Nagashima, J. W. McLean, R. M. Lawn, J. Lipid Res., 29:1643 (1988); M. E. Pape, E. F. Rehber, K. R. Marotti, G. W. Melchior, Artheriosclerosis 11:1759 (1991); G. Su et al., J. Immunol., 153:743 (1994); P. W. Gray et al., J. Biol. Chem. 264: 9505 (1989); Albers et al., Biochem. Biophys. Acta, 1258:27 (1995); X. C. Jiang et al., Biochemistry, 34:7258 (1995); L. B. Agellon et al., Biochemistry, 29:1372 (1990); X. C. Jiang et al., J. Biol. Chem., 266:4631 (1991)] but only the four human sequences are shown. Residues that are completely conserved in all proteins are indicated below the sequence *; those which are highly conserved are indicated by •. The secondary structure of BPI is indicated above the sequences. The β strands are indicated by arrows; strands which make up the central β sheet are shown with gray arrows. Because of the β bulges and pronounced twisting, some of the β strands have one or more residues that do not show classical H-bonding patterns or ΦΨ angles; these breaks are indicated by \hat{ } above the strands. The a helices are shown as cylinders, and one-residue breaks in helices B and B′ are indicated with a vertical dashed line. The horizontal dashed line indicates the linker region. Peptides from BPI and LBP with the highest LPS-binding activity (Little, et al., J. Biol. Chem. 268: 1865 (1994); Taylor et al., J. Biol. Chem. 270: 17934 (1995)) are in bold italics. The disulfide bond is indicated by S-S. Residues with atoms within 4 Å of the NH₂-terminal lipid are highlighted with gray shading; residues within 4 Å of the COOH-terminal lipid are shown with white letters in black boxes.

FIG. 6 Block diagram of a computer system 102 that can be used to implement the present invention. The computer system 102 includes a processor 106 connected to a bus 104. Also connected to the bus 104 are a main memory 108 (preferably implemented as random access memory, RAM) and a variety of secondary storage memory 110, such as a hard drive 112, a removable medium storage device 114, a command device 118, and a visualization device, 120. Also included is a removable storage medium 116.

FIG. 7(A) A ribbon diagram of residues 1-456 of the 1.7 Å crystal structure of BPI. The NH₂-terminal domain is blue, and the COOH-terminal is red. (B) Superposition of the NH₂-terminal domain (blue) on the COOH-terminal domain (red). (C) Schematic drawing of BPI showing its elongated shape and two-domain structure. Residues 1 to 229 and 251 to 456 are shown.

FIG. 8 Illustration of three major categories of pairs of 3D-1D environmentally conserved positions with dissimilar residues: (A) Conserved structural roles; (B) Auxiliary structural roles; (C) Different structural roles.

FIG. 9 Examples (A), (B) and (C) of pairs of 3D-1D environmentally conserved residues with dissimilar residues and conserved structural roles from the BPI domain alignment. Residues and secondary structure elements from the NH₂-terminal domain are blue; residues and secondary structure elements from the COOH-terminal domain are red.

FIG. 10(A) Space-filling representation of BPI. The NH₂-terminal domain is shown on the left. (B) View after rotating (A) 90° about the long axis of the molecule. (C) Ball-and-stick model and space-filling representation of the interaction of 3D-1D environmentally conserved positions in the NH₂-terminal lipid-binding pocket. (D) Ball-and stick model and space-filling representation of the interaction of 3D-1D environmentally conserved positions in the COOH-terminal domain.

### DETAILED DESCRIPTION

The present invention provides methods for crystallizing a BPI protein product where the crystals diffract x-rays with sufficiently high resolution to allow determination of the three-dimensional structure of the BPI protein product, including atomic coordinates. The three-dimensional structure (e.g, as provided on computer readable media as described herein) is useful for rational drug design of BPI-related (and LBP-, CETP-, PLTP-related) mimetics and/or ligands. Specifically provided is a method for crystallizing a recombinant non-glycosylated human BPI analog holoprotein comprising a 456 amino acid sequence wherein the amino acid serine at position 351 has been changed to alanine. The three-dimensional structure is useful for modeling and/or synthesizing BPI-related mimetics or ligands. Such BPI-related mimetics or ligands are useful for treating, inhibiting or preventing BPI-modulated diseases.

The present invention thus includes methods of expressing, purifying and crystallizing a BPI protein product from suitable sources, such as eukaryotic cells or tissues. The present invention also provides crystallized BPI protein products by these methods. The crystallized BPI is analyzed by x-ray diffraction techniques to obtain high resolution diffraction patterns and atomic coordinates that are suitable for molecular modeling.

As used herein, “BPI protein product” or “BPI protein” includes naturally and recombinantly produced BPI protein; natural, synthetic, and recombinant biologically active polypeptide fragments of BPI protein; biologically active polypeptide variants of BPI protein or fragments thereof, including hybrid fusion proteins and dimers; biologically active polypeptide analogs of BPI protein or fragments or variants thereof, including cysteine-substituted analogs; and BPI-derived peptides. The BPI protein products for therapeutic or diagnostic uses may be generated and/or isolated by any means known in the art. U.S. Pat. No. 5,198,541, the disclosure of which is incorporated herein by reference, discloses recombinant genes encoding and methods for expression of BPI proteins including recombinant BPI holoprotein, referred to as rBPI (also referred to as rBPI₅₅ or simply rBPI₅₀) and recombinant fragments of BPI. U.S. patent application Ser. No. 07/885,501, now abandoned, and a continuation-in-part thereof, U.S. patent application Ser. No. 08/072,063, filed May 19, 1993, issued as U.S. Pat. No. 5,439,807 on Aug, 8, 1995 and corresponding PCT Application No. 93/04752 filed May 19, 1993, which are all incorporated herein by reference, disclose novel methods for the purification of recombinant BPI protein products expressed in and secreted from genetically transformed mammalian host cells in culture and discloses how one may produce large quantities of recombinant BPI products suitable for incorporation into stable, homogeneous pharmaceutical preparations.

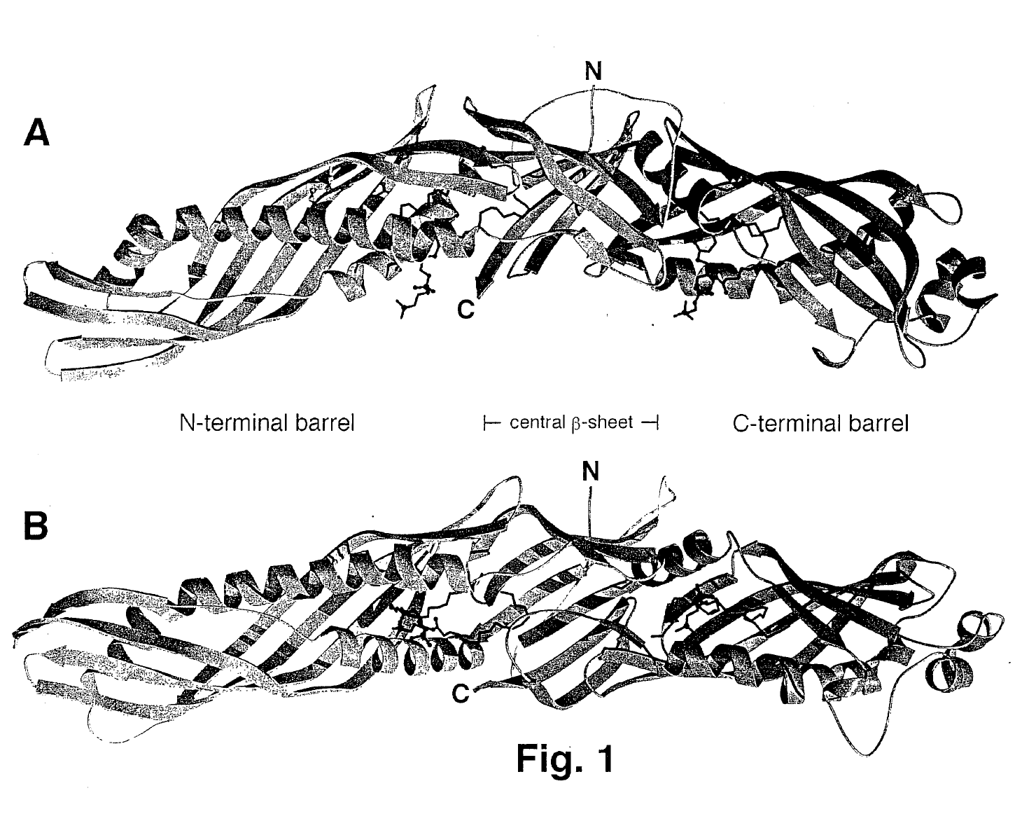

Biologically active fragments of BPI (BPI fragments) include biologically active molecules that have the same or similar amino acid sequence as a natural human BPI holoprotein, except that the fragment molecule lacks amino-terminal amino acids, internal amino acids, and/or carboxy-terminal amino acids of the holoprotein. Nonlimiting examples of such fragments include a N-terminal fragment of natural human BPI of approximately 25 kD, described in Ooi et al., J. Exp. Med., 174:649 (1991), and the recombinant expression product of DNA encoding N-terminal amino acids from 1 to about 193 or 199 of natural human BPI, described in Gazzano-Santoro et al., Infect. Immun. 60:4754-4761 (1992), and referred to as rBPI₂₃. In that publication, an expression vector was used as a source of DNA encoding a recombinant expression product (rBPI₂₃) having the 31-residue signal sequence and the first 199 amino acids of the N-terminus of the mature human BPI, as set out in FIG. 1 of Gray et al., supra, except that valine at position 151 is specified by GTG rather than GTC and residue 185 is glutamic acid (specified by GAG) rather than lysine (specified by AAG). Recombinant holoprotein (rBPI) has also been produced having the sequence (SEQ ID NOS: 1 and 2) set out in FIG. 1 of Gray et al., supra, with the exceptions noted for rBPI₂₃ and with the exception that residue 417 is alanine (specified by GCT) rather than valine (specified by GTT). Other examples include dimeric forms of BPI fragments, as described in U.S. Pat. No. 5,447,913, and corresponding PCT Application No. PCT/US95/03125, the disclosures of which are incorporated herein by reference. Preferred dimeric products include dimeric BPI protein products wherein the monomers are amino-terminal BPI fragments having the N-terminal residues from about 1 to 175 to about 1 to 199 of BPI holoprotein. A particularly preferred dimeric product is the dimeric form of the BPI fragment having N-terminal residues 1 through 193, designated rBPI₄₂ dimer.

Biologically active variants of BPI (BPI variants) include but are not limited to recombinant hybrid fusion proteins, comprising BPI holoprotein or biologically active fragment thereof and at least a portion of at least one other polypeptide, and dimeric forms of BPI variants. Examples of such hybrid fusion proteins and dimeric forms are described by Theofan et al. in U.S. patent application Ser. No. 07/885,911, now abandoned, and a continuation-in-part application thereof, U.S. patent application Ser. No. 08/064,693 filed May 19, 1993, issued as U.S. Pat. No. 5,643,570 on Jul. 1, 1997 and corresponding PCT Application No. US93/04754 filed May 19, 1993, which are all incorporated herein by reference and include hybrid fusion proteins comprising, at the amino-terminal end, a BPI protein or a biologically active fragment thereof and, at the carboxy-terminal end, at least one constant domain of an immunoglobulin heavy chain or allelic variant thereof. Similarly configured hybrid fusion proteins involving part or all Lipopolysaccharide Binding Protein (LBP) are also contemplated for use in the present invention.

Biologically active analogs of BPI (BPI analogs) include but are not limited to BPI protein products wherein one or more amino acid residues have been replaced by a different amino acid. For example, U.S. Pat. No. 5,420,019 and corresponding PCT Application No. US94/01235, filed Feb. 2, 1994, the disclosures of which are incorporated herein by reference, discloses polypeptide analogs of BPI and BPI fragments wherein a cysteine residue is replaced by a different amino acid. A preferred BPI protein product described by this application is the expression product of DNA encoding from amino acid 1 to approximately 193 or 199 of the N-terminal amino acids of BPI holoprotein, but wherein the cysteine at residue number 132 is substituted with alanine and is designated rBPI₂₁Δcys or rBPI₂₁. Other examples include dimeric forms of BPI analogs; e.g. U.S. patent application Ser. No. 08/212,132 filed Mar. 11, 1994, issued as U.S. Pat. No. 5,447,913 on Sep. 5, 1995 and corresponding PCT Application No. PCT/US95/03125, the disclosures of which are incorporated herein by reference.

Other BPI protein products useful according to the methods of the invention are peptides derived from or based on BPI produced by recombinant or synthetic means (BPI-derived peptides), such as those described in U.S. patent application Ser. No. 08/504,841 filed Jul. 20, 1995 and in PCT Application No. PCT/US94/10427 filed Sep. 15, 1994, which corresponds to U.S. patent application Ser. No. 08/306,473 filed Sep. 15, 1994, issued as U.S. Pat. No. 5,652,332 on Jul. 29, 1997, and PCT Application No. US94/02465 filed Mar. 11, 1994, which corresponds to U.S. patent application Ser. No. 08/209,762, filed Mar. 11, 1994, issued as U.S. Pat. No. 5,733,872 on Mar. 31, 1998, which is a continuation-in-part of U.S. patent application Ser. No. 08/183,222, filed Jan. 14, 1994, now abandoned, which is a continuation-in-part of U.S. patent application Ser. No. 08/093,202 filed Jul. 15, 1993, now abandoned, (for which the corresponding international application is PCT Application No. US94/02401 filed Mar. 11, 1994), which is a continuation-in-part of U.S. patent application Ser. No. 08/030,644 filed Mar. 12, 1993, issued as U.S. Pat. No. 5,348,942 on Sep. 20, 1994, the disclosures of all of which are incorporated herein by reference.

Presently preferred BPI protein products include recombinantly-produced N-terminal fragments of BPI, especially those having a molecular weight of approximately between 21 to 25 kD such as rBPI₂₃ or rBPI₂₁, or dimeric forms of these N-terminal fragments (e.g., rBPI₄₂ dimer). Additionally, preferred BPI protein products include rBPI₅₀ and BPI-derived peptides.

The administration of BPI protein products is preferably accomplished with a pharmaceutical composition comprising a BPI protein product and a pharmaceutically acceptable diluent, adjuvant, or carrier. The BPI protein product may be administered without or in conjunction with known surfactants, other chemotherapeutic agents or additional known anti-microbial agents. One pharmaceutical composition containing BPI protein products (e.g., rBPI₅₀, rRBPI₂₃) comprises the BPI protein product at a concentration of 1 mg/ml in citrate buffered saline (5 or 20 mM citrate, 150 mM NaCl, pH 5.0) comprising 0.1% by weight of poloxamer 188 (Pluronic F-68, BASF Wyandotte, Parsippany, N.J.) and 0.002% by weight of polysorbate 80 (Tween 80, ICI Americas Inc., Wilmington, Del.). Another pharmaceutical composition containing BPI protein products (e.g., rBPI₂₁) comprises the BPI protein product at a concentration of 2 mg/mL in 5 mM citrate, 150 mM NaCl, 0.2% poloxamer 188 and 0.002% polysorbate 80. Such combinations are described in PCT Application No. US94/01239 filed Feb. 2, 1994, which corresponds to U.S. patent application Ser. No. 08/190,869 filed Feb. 2, 1994, issued as U.S. Pat. No. 5,488,034 on Jan. 30, 1996, and U.S. patent application Ser. No. 08/012,360 filed Feb. 2, 1993, now abandoned, the disclosures of all of which are incorporated herein by reference. Additional formulations are provided in U.S. patent application Ser. Nos. 08/372,104, filed Jan. 13, 1995, now abandoned, 08/530,599, filed Sep. 19, 1995, now abandoned, and 08/586,133, filed Jan. 12, 1996 and corresponding WO96/21436 (PCT/US96/01095).

The x-ray diffraction patterns of the invention are now discovered to be of sufficiently high resolution to be useful for three-dimensional modeling of a BPI. Preferably the resolution is in the range of 1.5 to 3.5 Å, preferably 1.5-3.0 Å and more preferably ≦2.6 Å.

Three-dimensional modeling is performed using the diffraction coordinates from these x-ray diffraction patterns. The coordinates are entered into one or more computer programs for molecular modeling, as known in the art. Such molecular modeling can utilize known x-ray diffraction molecular modeling algorithms or molecular modeling software to generate atomic coordinates corresponding to the three-dimensional structure of at least one BPI or a fragment thereof.

The entry of the coordinates of the x-ray diffraction patterns and the amino acid sequence into such programs results in the calculation of most probable secondary, tertiary and quaternary structures of the protein, including overall atomic coordinates of a BPI or a fragment thereof. These structures are combined and refined by additional calculations using such programs to determine the probable or actual three-dimensional structure of the BPI, including potential or actual active or binding sites of the protein.

Such molecular modeling (and related) programs useful for rational drug design of ligands or mimetics, are also provided by the present invention. The drug design uses computer modeling programs which calculate how different molecules interact with the various sites of the BPI. This procedure determines potential ligands or mimetics of a BPI or at least one fragment thereof. The actual BPI-ligand complexes or mimetics are crystallized and analyzed using x-ray diffraction. The diffraction pattern coordinates are similarly used to calculate the three-dimensional interaction of a ligand and the BPI or a mimetic, in order to confirm that the ligand binds to, or changes the conformation of, a particular site on the BPI, or where the mimetic has a similar three-dimensional structure to that of a BPI or a fragment thereof.

The potential ligands or mimetics are then screened for activity relating to a BPI. Such screening methods are selected from assays for at least one biological activity of the native BPI.

The resulting ligands or mimetics, provided by methods of the present invention, are useful for treating, screening or preventing bacterial infections in animals, such as mammals (including humans) and birds. Mimetics or ligands of a particular BPI will similarly react with other BPIs from other species, subgenera or genera of the BPI source organism.

Also provided are biologically active BPI proteins. A BPI protein is also provided as a crystallized protein suitable for x-ray diffraction analysis. The x-ray diffraction patterns obtained by the x-ray analysis are of moderately high to high resolution, e.g., 1.5-3.5 Å. The coordinates from these diffraction patterns are suitable and useful for three-dimensional modeling of the crystallized protein.

During the three-dimensional modeling of the BPI, these coordinates are entered with the BPI amino acid sequence into computer modeling programs to generate secondary, tertiary and quaternary structures of the BPI, as atomic coordinates. These structures together provide the three-dimensional structure of the BPI. The calculated and confirmed three-dimensional structure is then used for rational drug design of ligands or mimetics of the BPI or a fragment thereof.

The determination of the three-dimensional structure of a BPI protein thus has a broad-based utility. Significant sequence identity and conservation of important structural elements is expected to exist among the BPI proteins of a particular species, subgenus, genus, or family. Therefore, the three-dimensional structure from one or a few BPI proteins can be used to identify therapeutics with one or more of the biological activities of BPI (and/or those of related proteins such as LBP, CETP and PLTP).

Determination of Protein Structures

Different techniques give different and complementary information about protein structure. The primary structure is obtained by biochemical methods, either by direct determination of the amino acid sequence from the protein, or from the nucleotide sequence of the corresponding gene or cDNA. The quaternary structure of large proteins or aggregates can also be determined by electron microscopy. To obtain the secondary and tertiary structure, which requires detailed information about the arrangement of atoms within a protein, x-ray crystallography is preferred.

The first prerequisite for solving the three-dimensional structure of a protein by x-ray crystallography is a well-ordered crystal that will diffract x-rays strongly. The crystallographic method directs a beam of x-rays onto a regular, repeating array of many identical molecules so that the x-rays are diffracted from it in a pattern from which the structure of an individual molecule can be retrieved. Well-ordered crystals of globular protein molecules are large, spherical, or ellipsoidal objects with irregular surfaces, and crystals thereof contain large holes or channels that are formed between the individual molecules. These channels, which usually occupy more than half the volume of the crystal, are filled with disordered solvent molecules. The protein molecules are in contact with each other at only a few small regions. This is one reason why structures of proteins determined by x-ray crystallography are generally the same as those for the proteins in solution.

The formation of crystals is dependent on a number of different parameters, including pH, temperature, protein, concentration, the nature of the solvent and precipitant, as well as the presence of added ions or ligands to the protein. Many routine crystallization experiments may be needed to screen all these parameters for the few combinations that might give crystal suitable for x-ray diffraction analysis. Crystallization robots can automate and speed up the work of reproducibly setting up large number of crystallization experiments.

A pure and homogeneous protein sample is important for successful crystallization. Proteins obtained from cloned genes in efficient expression vectors can be purified quickly to homogeneity in large quantities in a few purification steps. A protein to be crystallized is preferably at least 93-99% pure according to standard criteria of homogeneity. Crystals form when molecules are precipitated very slowly from supersaturated solutions. The most frequently used procedure for making protein crystals is the hanging-drop method, in which a drop of protein solution is brought very gradually to supersaturation by loss of water from the droplet to the larger reservoir that contains salt or polyethylene glycol solution.

Different crystal forms can be more or less well-ordered and hence give diffraction patterns of different quality. As a general rule, the more closely the protein molecules pack, and consequently the less water the crystals contain, the better is the diffraction pattern because the molecules are better ordered in the crystal.

X-rays are electromagnetic radiation at short wavelengths, emitted when electrons jump from a higher to a lower energy state. In conventional sources in the laboratory, x-rays are produced by high-voltage tubes in which a metal plate, the anode, is bombarded with accelerating electrons and thereby caused to emit x-rays of a specific wavelength, so-called monochromatic x-rays. The high voltage rapidly heats up the metal plate, which therefore has to be cooled. Efficient cooling is achieved by so-called rotating anode x-ray generators, where the metal plate revolves during the experiment so that different parts are heated up.

More powerful x-ray beams can be produced in synchrotron storage rings where electrons (or positrons) travel close to the speed of light. These particles emit very strong radiation at all wavelengths from short gamma rays to visible light. When used as an x-ray source, only radiation within a window of suitable wavelengths is channeled from the storage ring. Polychromatic x-ray beams are produced by having a broad window that allows through x-ray radiation with wavelengths of 0.2-3.5 Å.

In diffraction experiments a narrow and parallel beam of x-rays is taken out from the x-ray source and directed onto the crystal to produce diffracted beams. The incident primary beam causes damage to both protein and solvent molecules. The crystal is, therefore, usually cooled to prolong its lifetime (e.g., −220 to −50° C.). The primary beam must strike the crystal from many different directions to produce all possible diffraction spots, and so the crystal is rotated in the beam during the experiment.

The diffracted spots are recorded either on a film, the classical method, or by an electronic detector. The exposed film has to be measured and digitized by a scanning device, whereas electronic detectors feed the signals they detect directly in a digitized form into a computer. Electronic area detectors (an electronic film) significantly reduce the time required to collect and measure diffraction data.

When the primary beam from an x-ray source strikes the crystal, some of the x-rays interact with the electrons on each atom and cause them to oscillate. The oscillating electrons serve as a new source of x-rays, which are emitted in almost all directions, referred to as scattering. When atoms (and hence their electrons) are arranged in a regular three-dimensional array, as in a crystal, the x-rays emitted from the oscillating electrons interfere with one another. In most cases, these x-rays, colliding from different directions, cancel each other out; those from certain directions, however, will add together to produce diffracted beams of radiation that can be recorded as a pattern on a photographic plate or detector.

The diffraction pattern obtained in an x-ray experiment is related to the crystal that caused the diffraction. X-rays that are reflected from adjacent planes travel different distances, and diffraction only occurs when the difference in distance is equal to the wavelength of the x-ray beam. This distance is dependent on the reflection angle, which is equal to the angle between the primary beam and the planes.

The relationship between the reflection angle (θ), the distance between the planes (d), and the wavelength (λ) is given by Bragg's law: 2d sin θ=λ. This relation can be used to determine the size of the unit cell in the crystal. Briefly, the position on the film of the diffraction data relates each spot to a specific set of planes through the crystal. By using Bragg's law, these positions can be used to determine the size of the unit call.

Each atom in a crystal scatters x-rays in all directions, and only those that positively interfere with one another, according to Bragg's law, give rise to diffracted beams that can be recorded as a distinct diffraction spot above background. Each diffraction spot is the result of interference of all x-rays with the same diffraction angle emerging from all atoms. For example, for the protein crystal of myoglobin, each of the about 20,000 diffracted beams that have been measured contain scattered x-rays from each of the around 1500 atoms in the molecule. To extract information about individual atoms from such a system requires considerable computation. The mathematical tool that is used to handle such problems is called the Fourier transform.

Each diffracted beam, which is recorded as a spot on the film, is defined by three properties: the amplitude, which we can measure from the intensity of the spot; the wavelength, which is set by the x-ray source; and the phase, which is lost in x-ray experiments. All three properties are needed for all of the diffracted beams, in order to determine the position of the atoms giving rise to the diffracted beams.

For larger molecules, protein crystallographers have determined the phases in many cases using a method called multiple isomorphous replacement (MIR) (including heavy metal scattering), which requires the introduction of new x-ray scatterers into the unit cell of the crystal. These additions are usually heavy atoms (so that they make a significant contribution to the diffraction pattern), such that there should not be too many of them (so that their positions can be located); and they should not change the structure of the molecule or of the crystal cell, i. e., the crystals should be isomorphous. Isomorphous replacement is usually done by diffusing different heavy-metal complexes into the channels of the preformed protein crystals. The protein molecules expose side chains (such as SH groups) into these solvent channels that are able to bind heavy metals. It is also possible to replace endogenous light metals in metalloproteins with heavier ones, e.g., zinc by mercury, or calcium by samarium.

Since such heavy metals contain many more electrons than the light atoms (H, N, C, O and S) of the protein, they scatter x-rays more strongly. All diffracted beams would therefore increase in intensity after heavy-metal substitution if all interference were positive. In fact, however, some interference is negative; consequently, following heavy-metal substitution, some spots measurably increase in intensity, others decrease, and many show no detectable difference.

Phase differences between diffracted spots can be determined from intensity changes following heavy-metal substitution. First, the intensity differences are used to deduce the positions of the heavy atoms in the crystal unit cell. Fourier summations of these intensity differences give maps of the vectors between the heavy atoms, the so-called Patterson maps. From these vector maps the atomic arrangement of the heavy atoms is deduced. From the positions of the heavy metals in the unit cell, one can calculate the amplitudes and phases of their contribution to the diffracted beams of protein crystals containing heavy metals.

This knowledge is then used to find the phase of the contribution from the protein in the absence of the heavy-metal atoms. As both the phase and amplitude of the heavy metals and the amplitude of the protein alone is known, as well as the amplitude of the protein plus heavy metals (i.e., protein heavy-metal complex), one phase and three amplitudes are known. From this, the interference of the x-rays scattered by the heavy metals and protein can be calculated to see if it is constructive or destructive. The extent of positive or negative interference, with knowledge of the phase of the heavy metal, given an estimate of the phase of the protein. Because two different phase angles are determined and are equally good solutions, a second heavy-metal complex can be used which also gives two possible phase angles. Only one of these will have the same value as one of the two previous phase angles; it therefore represents the correct phase angle. In practice, more than two different heavy-metal complexes are usually made in order to give a reasonably good phase determination for all reflections. Each individual phase estimate contains experimental errors arising from errors in the measured amplitudes. Furthermore, for many reflections, the intensity differences are too small to measure after one particular isomorphous replacement, and others can be tried.

The amplitudes and the phases of the diffraction data from the protein crystals are used to calculate an electron-density map of the repeating unit of the crystal. This map then has to be interpreted as a polypeptide chain with a particular amino acid sequence. The interpretation of the electron-density map is made more complex by several limitations of the data. First of all, the map itself contains errors, mainly due to errors in the phase angles. In addition, the quality of the map depends on the resolution of the diffraction data, which in turn depends on how well-ordered the crystals are. This directly influences the image that can be produced. The resolution is measured in A units; the smaller this number is, the higher the resolution and therefore the greater the amount of detail that can be seen.

Building the initial model is a trial-and-error process. First, one has to decide how the polypeptide chain weaves its way through the electron-density map. The resulting chain trace constitutes a hypothesis, by which one tries to match the density of the side chains to the known sequence of the polypeptide. When a reasonable chain trace has finally been obtained, an initial model is built to give the best fit of the atoms to the electron density. Computer graphics are used both for chain tracing and for model building to present the data and manipulated the models.

The initial model will contain some errors. Provided the protein crystals diffract to high enough resolution (e.g., better than 3.5 Å), most or substantially all of the errors can be removed by crystallographic refinement of the model using computer algorithms. In this process, the model is changed to minimize the difference between the experimentally observed diffraction amplitudes and those calculated for a hypothetical crystal containing the model (instead of the real molecule). This difference is expressed as an R factor (residual disagreement) which is 0.0 for exact agreement and about 0.59 for total disagreement.

In general, the R factor is preferably between 0.15 and 0.35 (such as less than about 0.24-0.28) for a well-determined protein structure. The residual difference is a consequence of errors and imperfections in the data. These derive from various sources, including slight variations in the conformation of the protein molecules, as well as inaccurate corrections both for the presence of solvent and for differences in the orientation of the microcrystals from which the crystal is built. This means that the final model represents an average of molecules that are slightly different both in conformation and orientation.

In refined structures at high resolution, there are usually no major errors in the orientation of individual residues, and the estimated errors in atomic positions are usually around 0.1-0.2 Å, provided the amino acid sequence is known. Hydrogen bonds, both within the protein and to bound ligands, can be identified with a high degree of confidence.

Those of skill in the art understand that a set of structure coordinates determined by X-ray crystallography is not without standard error. For the purpose of this invention, any set of structure coordinates for a BPI protein that have a root mean square deviation of protein backbone atoms (N, Cα, C and O) of less that 0.75 | when superimposed—using backbone atoms—on the structure coordinates listed in Table 4 or in Table 6 shall be considered identical.

Most x-ray structures are determined to a resolution between 1.7 Å and 3.5 Å. Electron-density maps with this resolution range are preferably interpreted by fitting the known amino acid sequences into regions of electron density in which individual atoms are not resolved.

An amino acid sequence is preferred for accurate x-ray structure determination. Thus, recombinant DNA techniques have had a double impact on x-ray structural work. When a protein is cloned and overexpressed for structural studies, the amino acid sequence, necessary for the x-ray work, is also quickly obtained via the nucleotide sequence. Recombinant DNA techniques give us not only abundant supplies of rare proteins, but also their amino acid sequence as a bonus.

Overview of BPI Purification and Crystallization Methods

In general, a BPI protein is purified as described in Example 1. The resulting BPI is in sufficient purity and concentration for crystallization. The BPI is then isolated and assayed for biological activity and for lack of aggregation (which interferes with crystallization). The purified BPI preferably runs as a single band under reducing or nonreducing polyacrylamide gel electrophoresis (PAGE) (nonreducing is used to evaluate the presence of cysteine bridges).

The purified BPI is preferably crystallized using the hanging drop method under varying conditions of at least one of the following: pH, buffer type, buffer concentration, salt type, polymer type, polymer concentration, other precipitating agents and concentration of purified and cleaved BPI. See, e.g., the methods provided in a commercial kit, such as CRYSTAL SCREEN (Hampton Research, Riverside, Calif.). Differently sized and shaped crystals are tested for suitability for x-ray diffraction. Generally, larger crystals provide better crystallography than smaller crystals, and thicker crystals provide better diffraction than thinner crystals.

Purified BPIs

The results of the purification are optionally analyzed by polyacrylamide gel electrophoresis (PAGE) under reducing or non-reducing conditions. A single band is preferably obtained. With disulfide-containing BPIs, it is preferred that the analysis of the cleaved BPI be under non-reducing conditions to indicate whether the cleaved protein formed disulfide linked dimers. The amino acid sequence can also be determined according to known methods, or otherwise obtained, as this sequence is important in determining the three-dimensional structure of the cleaved protein (in combination with crystallographic analysis), as described herein, using molecular modeling techniques.

Before crystallization, the purified protein is tested for one or more of the known biological activities of a BPI protein.

It is preferred that the biological activity exceed the activity of the native protein. The preferred result indicates that the BPI protein retains its native structure, which is important for determining the three-dimensional crystal structure of the biologically active molecule. To identify the protease cleavage site, the purified and cleaved protein can be sequenced using known techniques. See, e.g., Murti et al., Proc. Natl. Acad. Sci. USA 90:1523-1525 (1993); Takimoto et al. (1992), infra, entirely incorporated herein by reference.

Protein Crystallization Methods

The hanging drop method is preferably used to crystallize the purified protein. See, e.g., Taylor et al., J. Mol. Biol. 226:1287-1290 (1992); Takimoto et al. (1992), infra; CRYSTAL SCREEN, Hampton Research.

A mixture of the purified protein and precipitant can include the following:

pH (e.g., 4-9);

buffer type (e.g., phosphate, cacodylate, acetates, imidazole, Tris HCl, sodium HEPES);

buffer concentration (e.g., 10-200 mM);

salt type (e.g., calcium chloride, sodium citrate, magnesium chloride, ammonium acetate, ammonium sulfate, potassium phosphate, magnesium acetate, zinc acetate; calcium acetate)

polymer type and concentration: (e.g., polyethylene glycol (PEG) 1-50%, average molecular weight 200-10,000);

other precipitating agents (salts: K, Na tartrate, NH₄SO₄, NaAc, LiSO₄, NaFormate, NaCitrate, MgFormate, NaPO₄, KPO₄ NH₄PO₄; organics: 2-propanol; non-volatile: 2-methyl-2,4-pentanediol); and

concentration of purified BPI (e.g., 1.0-100 mg/ml). See, e.g., CRYSTAL SCREEN, Hampton Research.

A non-limiting example of such crystallization conditions is the following:

purified protein (e.g., approximately 3-4 mg/ml);

H₂O;

precipitant 10-14% Polyethylene glycol (PEG) 8000 buffered with 100 mM cacodylate buffer and 200 mM of Mg acetate;

at an overall pH of about 3.5-8.5.

The above mixtures are used and screened by varying at least one of pH, buffer type; buffer concentration, precipitating salt type or concentration, PEG type, PEG concentration, and protein concentration. Crystals ranging in size from 0.2-0.7 mm are formed in 1-7 days. These crystals diffract x-rays to at least 3.5 Å resolution, such as 1.5-3.5 Å, or any range of value therein, such as 1.5, 1.6, 1.7, 1.8, 1.9, 2.0, 2.1, 2.2, 2.3, 2.4, 2.5, 2.6, 2.7, 2.8, 2.9, or 3.0, with 3.0 Å or less being preferred.

Protein Crystals

Crystals appear after 1-4 days and grow to maximal size within a week. From one ten crystals are observed in one drop and crystal forms can occur, such as, but not limited to, bipyramidal, rhomboid, and cubic. Initial x-ray analyses indicate that such crystals diffract at moderately high to high resolution. When fewer crystals are produced in a drop, they can be much larger size, e.g., 0.4-0.9 mm.

X-ray Crystallography Methods and Molecular Modeling

The crystals so produced for BPI are x-ray analyzed using a suitable x-ray source. Diffraction patterns are obtained. Crystals are preferably stable for at least 10 hrs in the x-ray beam. Frozen crystals (e.g., −220 to −50° C.) could also be used for longer x-ray exposures (e.g., 24-72 hrs), the crystals being relatively more stable to the x-rays in the frozen state. To collect the maximum number of useful reflections, multiple frames are optionally collected as the crystal is rotated in the x-ray beam, e.g., for 24-72 hrs. Larger crystals (>0.2 mm) are preferred, to increase the resolution of the x-ray diffraction. Alternatively, crystals may be analyzed using a synchrotron high energy x-ray source. Using frozen crystals, x-ray diffraction data is collected on crystals that diffract to a relatively high resolution of 3.5 ↑ or less, sufficient to solve the three-dimensional structure of BPI in considerable detail, as presented herein.

Native and/or derivative x-ray diffraction data with medium resolution is collected on area detectors mounted on rotating anode x-ray sources. The alternative program DENZO is preferably used for data processing and reduction (Sawyer et al., eds., Proceedings of CCP4 Study Weekend, pp. 56-62, SERC Darsbary Lab., UK (1993)).

The resolution is optionally improved using larger crystals, e.g., 0.2 mm, making data collection more efficient, particularly for the determination of suitable heavy metal derivatives, such as Hg, Pt, Pb, Ba, Cd, and/or La derivatives.

The heavy metal derivatives are used to determine the phase, e.g., by the isomorphous replacement method. Heavy atom isomorphous derivatives of BPI are used for x-ray crystallography, where the structure is solved using one or several derivatives, which, (when combined) improves the overall figure of merit. Derivatives are identified through Patterson maps and/or cross-phase difference Fourier maps, e.g., using the CCP4 package (SERC Collaborative Computing Project No. 4, Daresbury Laboratory, UK, 1979).

Phases were also obtained or improved by optimization of the anomalous dispersion component of the x-ray scattering which can break the phase ambiguity which a single heavy atom derivative gives. In certain cases phase information may be obtained without the need of a native set of data, through the use of multiple wavelength with anomalous dispersion phasing (MAD phasing). The wavelength of the x-rays used may be selected at a synchrotron source to optimize this anomalous scattering. In this case data from a derivatised crystal or crystals is collected at typically three wavelengths, two of which are very close to the absorption edge of the heavy atom scatterer. One way of obtaining a suitable heavy atom derivatised crystal is to derivatise a known ligand of the protein.

The program MLPHARE (Wolf et al., eds., Isomorphous Replacement and Anomalous Scattering: Proceedings of CCP4 Study Weekend, pp. 80-86, SERC Daresbury Lab., UK (1991)) is optionally used for refinement of the heavy atom parameters and the phases derived from them by comparing at least one of completeness (%), resolution (Å), R^{r} (%), heavy atom concentration (mM), soaking time, heavy atom sites, phasing power (acentric, centric) (See Table 1 as an analogous example from The Crystal Structure of diphtheria toxin, Choe et al., Nature 357: 216-222 (1992). Addition of heavy atom derivatives produce an MIR map with recognizable features.

The initial phases are calculated to 3.2 Å, and then improved and extended to a higher resolution of 2.8 Å (e.g., <3.0 Å) using solvent flattening, histogram matching and/or Sayre's equation in the program DM (Cowtan and Main, Acta Crystallogr. D 49:148-157 (1993)). The skeletonization of DM procedure is optionally used to improve connectivity in the bulk of the protein envelope. Both the MIR and density modified maps are optionally used in subsequent stages, to provide sufficient resolution and/or modeling of surface structures.

Skeletonized representations of electron density maps are then computed. These maps are automatically or manually edited using suitable software, e.g., the graphics package FRODO (Jones et al. (1991), infra) to give a continuous Cα trace. The BPI sequence is then aligned to the trace. Initially pieces of idealized polypeptide backbone were placed into regions of the electron density map with obvious secondary structures (e.g., α-helix, β-sheet). After a polyalanine model was constructed for the protein, amino acid sidechains were added where density was present in the maps. The amino acid sequence of BPI was then examined for regions with distinct sidechain patterns (e.g., three consecutive aromatic rings). When a pattern in the sequence was found to match an area of the map, the correct sidechains were built onto the existing model. Eventually fragments containing recognizable sequence motifs were connected into a single chain, completing the tracing of the amino acid sequence into the maps.

X-ray diffraction data (e.g., to ≦3.0 Å) was collected on an RAXIS 11 C area detector (e.g., a Mar imaging plate) mounted on a RIGAKU rotating anode or alternatively a synchrotron x-ray source, and processed using a suitable oscillation data reduction program (DENZO, Sawyer et al. eds., Proceedings of CCP4 Study Weekend, pp. 56-62, SERC Darsbary Lab., UK (1993). Cycles of simulated annealing against these data were refined using the program X-PLOR for molecular dynamics for R-factor refinement (X-PLOR, Brünger et al., J. Mol. Biol. 203:803-816 (1987)). This refinement was followed by manual rebuilding with FRODO using experimental and 2Fₒ-F_{c} maps. The model can be optionally further refined using a least-squares refinement program, such as TNT (Tronrud et al., Acta Crystallogr. A 43:489-501 (1987)).

One or more of the above modeling steps is performed to provide a molecular 3-D model of BPI. It is preferred that the BPI model has no residues in disallowed regions of the Ramachandran plot, and gives a positive 3D-1D profile (Luthy et al., Nature 356:83-85 (1992)), suggesting that all the residues are in acceptable environments (Kraulis (1991), infra).

Multiple isomorphous replacement phase determination was used for solving the three-dimensional structure of BPI. This structure is then used for rational drug design of BPI ligands or mimetics of at least one BPI bactericidal activity, or other biological activity important in inactivating bacterial toxicity, replication and/or infection.

Computer Related Embodiments

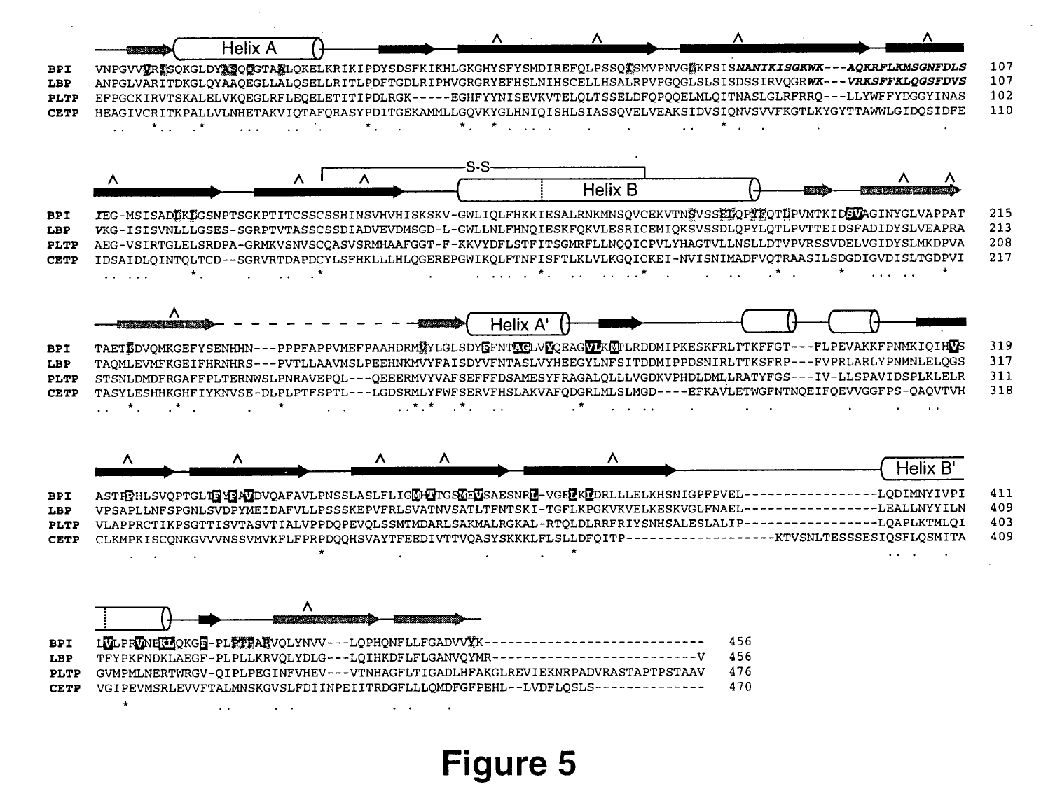

An amino acid sequence of a BPI protein (or related protein such as LBP, CETP or PLTP) and/or x-ray diffraction data, useful for computer molecular modeling of BPI protein (or related protein such as LBP, CETP or PLTP) or a portion thereof, can be “provided” in a variety of mediums to facilitate use thereof. As used herein, provided refers to a manufacture, which contains, for example, a BPI amino acid sequence and/or atomic coordinate/x-ray diffraction data of the present invention, e.g., an amino acid sequence provided in FIG. 5, a representative fragment thereof, or an amino acid sequence having at least 80-100% overall identity to an amino acid fragment of an amino acid sequence of FIG. 5 or a variant thereof. Such a method provides the amino acid sequence and/or x-ray diffraction data in a form which allows a skilled artisan to analyze and molecular model the three-dimensional structure of a BPI-related protein, including a subdomain thereof.

In one application of this embodiment, BPI (or related protein such as LBP, CETP or PLTP), or at least one subdomain thereof, amino acid sequence and/or x-ray diffraction data of the present invention is recorded on computer readable medium. As used herein, “computer readable medium” refers to any medium which can be read and accessed directly by a computer. Such media include, but are not limited to: magnetic storage media, such as floppy discs, hard disc storage medium, and magnetic tape; optical storage media such as optical discs or CD-ROM; electrical storage media such as RAM and ROM; and hybrids of these categories such as magnetic/optical storage media. A skilled artisan can readily appreciate how any of the presently known computer readable mediums can be used to create a manufacture comprising computer readable medium having recorded thereon an amino acid sequence and/or x-ray diffraction data of the present invention.

As used herein, “recorded” refers to a process for storing information on computer readable medium. A skilled artisan can readily adopt any of the presently know methods for recording information on computer readable medium to generate manufactures comprising an amino acid sequence and/or atomic coordinate/x-ray diffraction data information of the present invention.

A variety of data storage structures are available to a skilled artisan for creating a computer readable medium having recorded thereon an amino acid sequence and/or atomic coordinate/x-ray diffraction data of the present invention. The choice of the data storage structure will generally be based on the means chosen to access the stored information. In addition, a variety of data processor programs and formats can be used to store the sequence and x-ray data information of the present invention on computer readable medium. The sequence information can be represented in a word processing text file, formatted in commercially-available software such as WordPerfect and MICROSOFT Word, or represented in the form of an ASCII file, stored in a database application, such as DB2, Sybase, Oracle, or the like. A skilled artisan can readily adapt any number of dataprocessor structuring formats (e.g. text file or database) in order to obtain computer readable medium having recorded thereon the information of the present invention.

By providing computer readable medium having stored thereon a BPI or related sequence protein and/or atomic coordinates based on x-ray diffraction data, a skilled artisan can routinely access the sequence and atomic coordinate or x-ray diffraction data to model a BPI or related protein, a subdomain thereof, mimetic, or a ligand thereof. Computer algorithms are publicly and commercially available which allow a skilled artisan to access this data provided in a computer readable medium and analyze it for molecular modeling and/or RDD. See, e.g., Biotechnology Software Directory, MaryAnn Liebert Publ., New York (1995).

The present invention further provides systems, particularly computer-based systems, which contain the sequence and/or diffraction data described herein. Such systems are designed to do structure determination and RDD for a BPI or related protein or at least one subdomain thereof. Non-limiting examples are microcomputer workstations available from Silicon Graphics Incorporated and Sun Microsystems running UNIX based, Windows NT or IBM OS/2 operating systems.

As used herein, “a computer-based system” refers to the hardware means, software means, and data storage means used to analyze the sequence and/or x-ray diffraction data of the present invention. The minimum hardware means of the computer-based systems of the present invention comprises a central processing unit (CPU), input means, output means, and data storage means. A skilled artisan can readily appreciate which of the currently available computer-based systems are suitable for use in the present invention. A visualization device, such as a monitor, is optionally provided to visualize structure data.

As stated above, the computer-based systems of the present invention comprise a data storage means having stored therein a BPI or related protein or fragment sequence and/or atomic coordinate/x-ray diffraction data of the present invention and the necessary hardware means and software means for supporting and implementing an analysis means. As used herein, “data storage means” refers to memory which can store sequence or atomic coordinate/x-ray diffraction data of the present invention, or a memory access means which can access manufactures having recorded thereon the sequence or x-ray data of the present invention.

As used herein, “search means” or “analysis means” refers to one or more programs which are implemented on the computer-based system to compare a target sequence or target structural motif with the sequence or x-ray data stored within the data storage means. Search means are used to identify fragments or regions of a BPI or related protein which match a particular target sequence or target motif. A variety of known algorithms are disclosed publicly and a variety of commercially available software for conducting search means are and can be used in the computer-based systems of the present invention. A skilled artisan can readily recognize that any one of the available algorithms or implementing software packages for conducting computer analyses can be adapted for use in the present computer-based systems.

As used herein, “a target structural motif,” or “target motif,” refers to any rationally selected sequence or combination of sequences in which the sequence(s) are chosen based on a three-dimensional configuration or electron density map which is formed upon the folding of the target motif. There are a variety of target motifs known in the art. Protein target motifs include, but are not limited to, enzymic active sites, structural subdomains, epitopes, functional domains and signal sequences. A variety of structural formats for the input and output means can be used to input and output the information in the computer-based systems of the present invention.

A variety of comparing means can be used to compare a target sequence or target motif with the data storage means to identify structural motifs or electron density maps derived in part from the atomic coordinate/x-ray diffraction data. A skilled artisan can readily recognize that any one of the publicly available computer modeling programs can be used as the search means for the computer-based systems of the present invention.

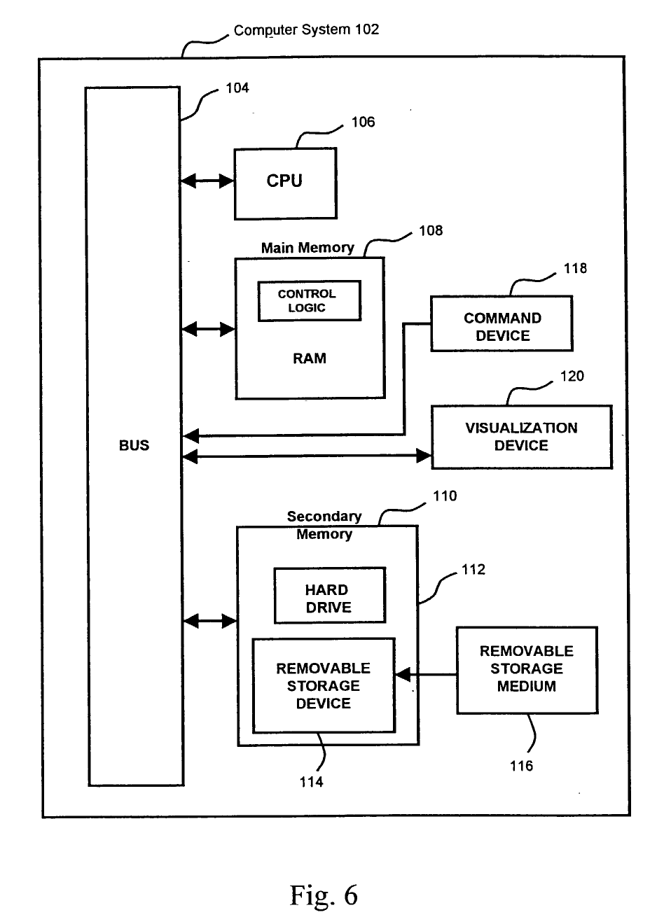

One application of this embodiment is provided in FIG. 6. FIG. 6 provides a block diagram of a computer system 102 that can be used to implement the present invention. The computer system 102 includes a processor 106 connected to a bus 104. Also connected to the bus 104 are a main memory 108 (preferably implemented as random access memory, RAM) and a variety of secondary storage memory 110, such as a hard drive 112, a removable medium storage device 114, a command device 118, and a visualization device, 120. Also included is a removable storage medium 116. The removable medium storage device 114 may represent, for example, a floppy disk drive, a CD-ROM drive, a magnetic tape drive, etc. A removable storage medium 116 (such as a floppy disk, a compact disk, a magnetic tape, etc.) containing control logic and/or data recorded therein may be inserted into the removable storage device 114. The computer system 102 includes appropriate software for reading the control logic and/or the data from the removable storage medium 116 once inserted in the removable medium storage device 114.

Amino acid, encoding nucleotide or other sequence and/or atomic coordinate/x-ray diffraction data of the present invention may be stored in a well known manner in the main memory 108, or any of the secondary storage devices 110, and/or a removable storage medium 116. Software for accessing and processing the amino acid sequence and/or atomic coordinate/x-ray diffraction data (such as search tools, comparing tools, etc.) reside in main memory 108 during execution. User commands are implemented through a command device 118, such as a keyboard. The visualization device 120 is optionally used to visualize the structure data.

Structure Determination

One or more computational steps, computer programs and/or computer algorithms are used to provide a molecular 3-D model of BPI or related protein, using amino acid sequence data from FIG. 5 (or fragments or variants thereof) and/or atomic coordinate/x-ray diffraction data. In x-ray crystallography, x-ray diffraction data and phases are combined to produce electron density maps in which the three-dimensional structure of a BPI protein is then built or modeled. MIR Phase determination was used for solving the three-dimensional structure of BPI. This structure can then be used for RDD of mimetics or ligands of a BPI or related protein and its associated biological activity, which is relevant to a protein modulated disease.

Density Modification and Map Interpretation

Electron density maps were calculated by X-PLOR or alternatively using such programs as those from the CCP4 computing package (SERC (UK) Collaborative Computing Project 4, Daresbury Laboratory, UK, 1979). If non-crystallographic symmetry axes are present, cycles of symmetry averaging can further be used, such as with the program RAVE (Kleywegt & Jones, Bailey et al., eds., First Map to Final Model, SERC Daresbury Laboratory, UK, pp 59-66 (1994)) and gradual model expansion. For map visualization and model building the program FRODO was used or alternatively, a program such as “O” (Jones (1991), infra) can be used.

Refinement and Model Validation

Rigid body and positional refinement can be carried out using a program such as X-PLOR (Brünger (1992), infra), e.g., with the stereochemical parameters of Engh and Huber (Acta Ctyst. A47:392-400 (1991)). If the model at this stage in the averaged maps is still missing residues (e.g., at least 5-10 per subunit), some or all of the missing residues can be incorporated in the model during additional cycles of positional refinement and model building. The refinement procedure can start using data from lower resolution (e.g., 25-10 Å to 10-3.0 Å and then be gradually extended to include higher resolution data from 12-6Å to 3.0-1.5 Å). B-values (also termed temperature factors) for individual atoms were refined once data between 2.9 and 1.5 Å has been added. Subsequently waters were gradually added by manual inspection of electron density maps. Alternatively, a program such as ARP (Lamzin and Wilson, Acta Cryst. D49:129-147 (1993)) can be used to add crystallographic waters and as a tool to check for bad areas in the model. The programs PROCHECK (Lackowski et al., J. Appl. Cryst. 26:283-291 (1993)), WHATIF (Vriend, J. Mol. Graph. 8:52-56 (1990)), PROFILE 3D (Lüthy et al., Nature 356:83-85 (1992)), and ERRAT (Colovos & Yeates Protein Science, 2:1511-19 (1993)) as well as the geometrical analysis generated by X-PLOR were used to check the structure for errors. Anisotropic scaling between F_{obs} and F_{calc} was applied after careful assessment of the quality and completeness of the data.

The program DSSP was used to assign the secondary structure elements (Kabsch and Sander, Biopolymers, 22:2577-2637 (1983)). A program such as SUPPOS (from the BIOMOL crystallographic computing package) can be used for some or all of the least-squares superpositions of various models and parts of models. The program ALIGN (Cohen J. Mol. Biol., 190: 593-604 (1986)) was used to superimpose N- and C-terminal domains of BPI. Solvent accessible surfaces and electrostatic potentials can be calculated using such programs as GRASP (Nicholls et al. (1991), infra).

The structure of BPI from different organisms and the related proteins LBP, CETP and PLTP can thus be solved with the molecular replacement procedure such as by using X-PLOR (Brünger (1992), infra). A partial search model for a portion or all of these proteins can be constructed using the structures of BPI. The rotation and translation function can be used to yield orientations and positions for these models. Symmetry averaging can also be done using the RAVE program and model expansion can also be used to add missing residues resulting in a model with 95-99.9% of the total number of residues. The model can be refined in a program such as X-PLOR (Brünger (1992), supra), to a suitable crystallographic R_{factor}. The model data is then saved on computer readable medium for use in further analysis, such as rational drug design.

Rational Design of Mimetics or Ligands

The determination of the crystal structure of a BPI protein, as described herein, provides a basis for the design of new and specific agents, including proteins or organic compounds.

Several approaches can be taken for the use of the crystal structure of a BPI in the rational design of protein or organic analogs having a relevant activity similar to that of a BPI or related protein. A computer-assisted, manual examination of a BPI potential binding site structure is optionally done. The use of software such as GRID—Goodford, J. Med. Chem. 28:849-857 (1985) a program that determines probable interaction sites between probes with various functional group characteristics and the protein surface—is used to analyze the surface sites to determine structures of similar inhibiting proteins or compounds. The GRID calculations, with suitable inhibiting groups on molecules (e.g., protonated primary amines) as the probe, are used to identify potential hotspots around accessible positions at suitable energy contour levels.

A diagnostic or therapeutic BPI or related protein modulating ligand of the present invention can be, but is not limited to, at least one selected from a lipid, a nucleic acid, a compound, a protein, an element, an antibody, a saccharide, an isotope, a carbohydrate, an imaging agent, a lipoprotein, a glycoprotein, an enzyme, a detectable probe, and antibody or fragment thereof, or any combination thereof, which can be detectably labeled as for labeling antibodies. Such labels include, but are not limited to, enzymatic labels, radioisotope or radioactive compounds or elements, fluorescent compounds or metals, chemiluminescent compounds and bioluminescent compounds. Alternatively, any other known diagnostic or therapeutic agent can be used in a method of the invention. Suitable compounds are then tested for activities of a BPI protein or BPI mimetic.

The program DOCK (Kuntz et al. J. Mol. Biol., 161:269-288 (1982)) may be used to analyze an active site or ligand binding site and suggest ligands with complementary steric properties. Several methodologies for searching three-dimensional databases to test pharmacophore hypotheses and select compounds for screening are available. These include the program CAVEAT (Bacon et al. J. Mol Biol., 225: 849-858 (1992)) which uses databases of cyclic compounds which can act as “spacers” to connect any number of chemical fragments already positioned in the active site. This allows one skilled in the art to quickly generate hundreds of possible ways to connect the fragments already known or suspected to be necessary for tight binding. The program LUDI (Bohm et al. J. Comput. -Aid. Mol. Des., 6:61-78 (1992)) can determine a list of interactions sites into which to place both hydrogen bonding and hydrophobic fragments. LUDI then uses a library of approx. 600 linkers to connect up to four different interaction sites into fragments. Then smaller “bridging” groups such as —CH2— and —COO— are used to connect these fragments. For example, for the enzyme DHFR, the placements of key functional groups in the well-known inhibitor methotrexate were reproduced by LUDI. See also, Rotstein and Murcko, J. Med. Chem., 36:1700-1710 (1992)).

After preliminary experiments are done to determine the Kₜ of a ligand (e.g., a lipid ligand) by BPI (or related) protein to a BPI (or related) protein, mimetic or fragment, the time-dependent nature of the inhibition by the BPI or related protein (e.g., by the method of Henderson (Biochem. J. 127:321-333 (1972)) is determined.

For example, a lipid ligand and a BPI mimetic are pre-incubated in buffer. Reactions are initiated by the addition of detecting substrate. Aliquots are removed over a suitable time course and each quenched by addition into the aliquots of suitable quenching solution. The concentration of product are determined by known methods of detection. Plots of activity against time can be close to linear over the assay period, and are used to obtain values for the initial velocity in the presence (V₁) or absence (V₀) of, for example, a BPI mimetic. Error is present in both axes in a Henderson plot, making it inappropriate for standard regression analysis (Leatherbarrow, Trends Biochem. Sci. 15:455-458 (1990)). Therefore, K₁ values are obtained from the data by fitting to a modified version of the Henderson equation for competitive inhibition:

Qr²+(Eₜ−Q−Iₜ)r−Eₜ=0

where (using the notation of Henderson (Biochem. J. 127:321-333 (1972)):

$$Q = {K}_{t} ( \frac{{A}_{t} + {K}_{a}}{{K}_{a}} ) and r = \frac{{V}_{o}}{{V}_{i}}$$

This equation is solved for the positive root with the constraint that Q=Kₜ((Aₜ+Kₐ)/Kₐ) using PROCNLIN from SAS (SAS Institute Inc., Cary, N.C., USA) which performs nonlinear regression using least-square techniques. The iterative method used is optionally the multivariate secant method, similar to the Gauss-Newton method, except that the derivatives in the Taylor series are estimated from the histogram of iterations rather than supplied analytically. A suitable convergence criterion is optionally used, e.g., where there is a change in loss function of less than 10⁻⁸.

Once modulating compounds are found, crystallographic studies of co-complexes, for example, BPI mimetics complexed to a ligand are performed. As used herein, a co-complex refers to a BPI protein, fragment, analog or variant thereof in covalent or non-covalent association with a chemical entity or compound. As a non-limiting example, BPI crystals are soaked for 2 days in 0.01-100 mM inhibitor compound and x-ray diffraction data are collected on an area detector and/or an image plate detector (e.g., a Mar image plate detector) using a rotating anode x-ray source. Data are collected to as high a resolution as possible, e.g., ≦3.0 Å, and merged with a suitable R-factor on intensities. An atomic model of the mimetic is built into the difference Fourier map (F_{inhibitor complex}−Fₙₐₜᵢᵥₑ). The model can be refined to convergence in a cycle of simulated annealing (Brünger (1987), infra) involving 10-100 cycles of energy refinement, 100-10,000 1-fs steps of room temperature dynamics and/or 10-100 more cycles of energy refinement. Harmonic restraints may be used for the atom refinement, except for atoms within a 10-15 Å radius of the inhibitor. An R-factor is calculated for the model as well as an r.m.s. deviation from the ideal bond lengths and angles.

Direct measurements of activity provide further confirmation that the modeled mimetic compounds are high-affinity inhibitors for the lipid ligands. Other suitable assays for biological activity known for BPI or related proteins may be used.

Preferably, little or no change in the structure of the BPI or mimetic occurs in the electron density map described above. Kₜ values are determined by a previously described method (Henderson (1972), infra) to evaluate mimetic proteins or organic compounds.

Atomic coordinates of BPI proteins are useful in the generation of molecular models of related proteins and of BPI mimetics. The atomic coordinates generated from the solved three-dimensional structure of BPI disclosed herein may be utilized in combination with additional structural and/or physicochemical information, such as amino acid sequence data, x-ray diffraction data, combinations of x-ray diffraction data from multiple isomorphous replacement molecular replacement techniques, or other phase determination techniques. These combinations may be used to generate other three-dimensional coordinate data useful to generate secondary, tertiary and/or quaternary structures and/or domains of BPI or related proteins, including BPI-related lipid binding proteins, or their fragments, analogs, or variants. These alternate coordinate sets are useful to provide overall three-dimensional structure, as well as binding and or active sites of a BPI or related protein, including BPI-related lipid binding proteins, or their fragments, analogs, or variants. These alternate coordinate data sets are also useful in molecular modeling computer-based systems and methods for rational drug design of mimetics and ligands of BPI and other related proteins, including other BPI-related lipid binding proteins. Utilizing CLUSTAL (a multiple sequence alignment program in PC-Gene) and the Homology module (a structure-based homology modeling program in Insightll on a Silicon Graphics Incorporated workstation, molecular models (and the corresponding three-dimensional coordinates files) of lipopolysaccharide binding protein (LBP), cholesteryl ester transfer protein (CETP) and phospholipid transfer protein (PLTP) are generated. With these files, existing mutants are mapped and new ones designed.

The results described herein demonstrate that tight-binding mimetics of a BPI or related protein, based on the crystal structure of BPI, are provided by the present invention. Demonstration of clinically relevant levels of a biological activity of the mimetic is also useful.

In evaluating mimetics for biological activity in animal models (e.g., mouse, rat, rabbit, baboon) various oral and parenteral routes of administration are evaluated. Using this approach, it is expected that a biological activity occurs in suitable animal models, e.g., using the inimetics discovered by structure determination and x-ray crystallography.

Having now generally described the invention, the same will be more readily understood through reference to the following examples which are provided by way of illustration, and are not intended to be limiting of the present invention.

### EXAMPLE 1

### Preparation and Purification for Crystallization Construction of Plasmids Containing BPI (S351A)

BPI contains a single N-linked glycosylation site at the asparagine at position 349 which was eliminated by genetic engineering of the DNA sequence of BPI as follows. For glycosylation to occur at this position, the asparagine must occur within the sequence Asn-X-Ser/Thr where X can be any amino acid, except proline. N-linked glycosylation can be eliminated by either changing the Asn to another amino acid such as glutamine or by changing the serine or threonine to an alternate amino acid. The latter strategy was used to construct vectors containing BPI with an alanine at position 351 instead of serine.

Construction of Plasmids for BPI Expression

The plasmid pIC108 containing a cDNA encoding BPI cloned in a T3T7 plasmid (Clontech, Palo Alto, Calif.) served as the starting point for the construction of a vector for expression of nonglycosylated rBPI in mammalian cells.

To allow insertion of BPI into an optimized mammalian expression vector, a unique XhoI site was first added to the 3′ end of the BPI gene in pIC108. Two oligonucleotides were synthesized for this purpose: BPI-53 (5′ ACT GGT TCC ATG GAG GTC AGC GCC 3′) encoding amino acids 361-370 of BPI and BPI-54 (5′ GAC AGA TCT CTC GAG TCA TTT ATA GAC AA 3′) encoding the last four amino acids of coding sequence, the stop codon (TGA), and incorporating an XhoI site immediately downstream of the stop codon. These oligonucleotides were used to PCR amplify a 280 bp fragment of the C-terminus of BPI and incorporate the XhoI site at the 3′ end of the gene. The amplified fragment was digested with NcoI and BglII and ligated to a ˜4100 bp NcoI-BamHI fragment from pIC108 to generate the plasmid pSS101.

Construction of Plasmid with BPI (S351A)

The glycosylation site was next removed by replacing the region from a unique XcmI site to a unique SphI site within the BPI gene in pSS101 with an annealed oligonucleotide that contained the codon (TCC) for the serine at amino acid position 351 changed to the codon (GCC) for alanine as shown below.

```
Wild type
                XcmI SphI
. . . CCC AAC TCC TCC CTG GCT TCC CTC TTC CTG ATT GGC ATG CAC
. . . GGG TTC AGG AGG GAC CGA AGG GAG AAG GAC TAA CCG TAC GTG
            Pro Asn Ser Ser Leu Ala Ser Leu Phe Leu Ile Gly Met His
                                    351
Nonglycosylated
                XcmI SphI
. . . CCC AAC TCC GCC CTG GCT TCC CTC TTC CTG ATT GGC ATG CAC
. . . GGG TTC AGG CGG GAC CGA AGG GAG AAG GAC TAA CCG TAC GTG
            Pro Asn Ser Ala Leu Ala Ser Leu Phe Leu Ile Gly Met His
                                    351
```

This step generated the plasmid pSS102.

To construct the vector, pING4322, for the expression of full length nonglycosylated holo BPI in mammalian cells, pSS102 was digested with BstBI and XhoI and a 596 bp fragment, which included the modified BPI sequence, was purified and ligated to the large BstBI-XhoI fragment from pING4147 which contains the gpt gene encoding resistance to mycophenolic acid, the human Ig enhancer, the human cytomegalovirus promoter (CMV) and the mouse light chain 3′ untranslated region and is identical to the vector, pING4144 as described in U.S. Pat. No. 5,420,019 and WO94/18323 (PCT/US94/01235) hereby incorporated by reference, except that it contains the codon for the native cysteine instead of an alanine at amino acid position 132 of the BPI gene.

Stable Transfection of Mammalian Cells for Expression of Nonglycosylated BPI

Mammalian cells are preferred hosts for production of rBPI protein analogs as described herein. Such cells permit proper secretion, folding, and post-translational modification of expressed proteins. Presently preferred mammalian host cells for production of BPI proteins include cells of fibroblast origin, such as CHO-K1 cells (ATCC CCL61), CHO-DG44 cells (a dihydrofolate reductase [DHFR] minus mutant of CHO Toronto obtained from Dr. Lawrence Chasin, Columbia University), CHO-DXB-11 (a DHFR⁻ mutant of CHO-K1 obtained from Dr. Lawrence Chasin), Vero Cells (ATCC CRL81) and Baby Hamster Kidney (BHK) cells (ATCC CRL6281) and cells of lymphoid origin, such as the hybridoma Sp2/O-Ag14 (ATCC CRL1581) or the myeloma, NSO (ECACC No. 85110503).

Transfection of mammalian cells can be accomplished by a variety of methods. Two of the most common approaches involve calcium phosphate precipitation of the expression vector DNA which is subsequently taken by the cells and electroporation, which causes the cells to take up the DNA through membrane pores created by the generation of a strong electric field. Selection for transfected cells is facilitated by the incorporation in the expression vector of a gene whose product allows the transfected cells to survive and grow under selective conditions. A number of such genes have been identified including, among others, the bacterial Tn5 neo gene, which encodes resistance to the antibiotic G418 and the Escherichia coli guanine phosphoribosyl transferase (gpt) gene, which encodes resistance to mycophenolic acid (MPA) in the presence of xanthine (Mulligan and Berg, Proc. Natl. Acad. Sci. 78:2072-2076 (1981)), the dihydrofolate reductase (DHFR) gene, which allows for growth of DHFR⁻ cells in the absence of nucleosides and gene amplification in the presence of increasing concentrations of methotrexate, the glutamine synthetase gene, which allows for growth of glutamine auxotrophs without glutamine and gene amplification in the presence of methionine sulfoximine and the Salmonella typhimurium hisD gene and the E. coli trpB gene (Hartman and Mulligan, Proc. Natl. Acad. Sci. 85: 8047-8051 (1988)), which allow growth in the presence of histidinol or without tryptophan (in the presence of indole), respectively. The availability of these selective markers provide significant flexibility for the generation of mammalian cell lines that express recombinant products, since they can be used either alone or in various combinations to provide cell lines with the highest possible productivity.

Transfection of CHO-K1 Cells with pING4322

The CHO-KI cell line was maintained in Ham's F12 medium plus 10% fetal bovine serum (FBS). Media were supplemented with glutamine/penicillin/strepto-mycin (Irvine Scientific, Irvine, Calif.).

CHO-K1 cells were transfected by electroporation with 40 μg of pING4322 DNA that was first digested with PvuI, phenol-chloroform extracted and ethanol precipitated. Following the electroporation, the cells were allowed to recover for 24 hours in non-selective Ham's F12 medium. The cells were then trypsinized, resuspended at a concentration of ˜5×10⁴ cells/ml in Ham's F12 medium supplemented with MPA (25 μg/mL) and xanthine (250 μg/mL) and plated at ˜10⁴ cells/well in 96 well plates. Untransfected CHO-K1 cells are unable to grow in this medium due to the inhibition of pyrimidine synthesis by the MPA. At ˜2 weeks, colonies consisting of transfected cells were observed in the 96 well plates. Supernatants from wells containing single colonies were analyzed for the presence of BPI-reactive protein by anti-BPI ELISA using BPI₂₃ as a standard. In this assay, Immulon-II 96 well plates (Dynatech) were pre-coated with affinity purified rabbit anti-BPI₂₃ antiserum, followed by supernatant samples and detection was with affinity purified, biotinylated rabbit anti-BPI₂₃ antiserum followed by peroxidase-labeled avidin. A total of 100 colonies were screened in this manner. The top isolates were transferred to 24 well plates and productivity was assessed as follows. Cells were grown to confluence in a 24 well plate in Ham's F12 medium supplemented with 10% FBS. Once the cells reached confluence, the Ham's F12 medium was removed and 2 ml of HB-CHO serum free medium (Irvine Scientific) plus 40 μL of S-Sepharose beads (Pharmacia) were added. The cells were incubated for 7 days after which the S-Sepharose beads were removed and washed with 0.1 M NaCl in 10 mM Tris buffer (pH 7.5). BPI was eluted from the beads by addition of 1.0 M NaCl in Tris buffer. The top producers, designated Clones 37 and 91, secreted ˜17 and 14 μg/ml, respectively in this assay and were frozen as Research Cell Bank numbers C2020 and C2021, respectively. Purified protein was prepared for crystallization studies as follows.

Production and Purification of Nonglycosylated rBPI

The host cells used to prepare protein for crystallization studies were CHO-K1 cells transformed with the DNA vector pING4322 which includes DNA encoding the 456 amino acids of human BPI preceded by its endogenous 31 residue secretory signal as described above. During post-translational secretory processing, the signal sequence residues were removed by the host cell. The desired expression product, nonglycosylated rBPI, was a biologically active variant of the human BPI molecule in which the amino acid serine at position 351 in the human BPI protein has been replaced with an alanine.

Forty roller bottles were prepared which contained the transfected CHO-K1 host cells at 1.3×10⁷ cells per bottle in DME/F12 media supplemented with 5% fetal bovine serum (FBS). The cells were grown for three days, at which time 500 ml of fresh media, DME/F12 with 2.5% FBS was added along with a 10 ml slurry (approximately 8 gr.) of sterilized S-Sepharose (Pharmacia, fast flow #17-0511-01, Uppsula, Sweden) and 1 ml of a 1 M solution of sodium butyrate. After two days, the old media plus the S-Sepharose was removed and fresh media, S-Sepharose and sodium butyrate were added to each roller bottle. This process of harvesting the expressed protein product with S-Sepharose was repeated for a total of three harvests, and the S-Sepharose removed during each harvest was pooled. The use of S-Sepharose beads to capture recombinant BPI protein products has been described in U.S. Pat. No. 5,439,807 and WO93/23540 (PCT/US93/04752).

The expressed nonglycosylated rBPI protein was purified from the pooled S-Sepharose by first removing it from the S-Sepharose resin followed by further purification and concentration on a series of Q-Sepharose (Pharmacia, fast flow #17-0510-01) and CM-Spherodex (Sepracor, #273431, Villeneuve la Garenne, France) columns. Following purification, the protein was buffer exchanged utilizing a Sephacryl S-100 (Pharmacia, high resolution #17-0612-01) column. Specifically, the pooled S-Sepharose resin from each harvest was allowed to settle for approximately 15 minutes. The media was removed by decanting and the settled resin was washed three times with approximately 400 ml of 20 mM MES, pH 6.8, 150 mM NaCl. For each wash, the buffer was added, the mixture was stirred gently and the resin was allowed to resettle for approximately 15 minutes. Each buffer wash was removed by decanting. The beads were then washed with 400 mL of 20 mM sodium acetate/acetic acid, 150 mM NaCl, pH 4.0 (acetate buffer), and then poured into a 2.5×50 cm liquid chromatography column (BIORAD, Econocolumn, Richmond, Calif.). The column was washed extensively with approximately 2 liters of 400 mM NaCl-acetate buffer, pH 4.0 until the A280 absorbance reading of the column eluate was equal to that of the buffer alone. The column was additionally washed with approximately 600 ml of 600 mM NaCl-acetate buffer until the A280 absorbance of the eluate was again equal to that of buffer alone. The protein was then eluted from the S-Sepharose in approximately 500 ml of 1.0 M NaCl-acetate buffer.

The S-Sepharose eluates from each harvest were pooled and diluted to a NaCl concentration of 300 mM. The diluted material was then loaded on to a two column, serial arrangement of a 100 ml Q-Sepharose column connected to a 12 ml CM-Spherodex column. Both columns were constructed using new, sterile resin and were pre-equilibrated with 20 mM MES, pH 5.5, 200 mM NaCl. The Q-Sepharose column served to remove any nucleic acid in the sample material, and the protein did not bind to this resin. After the approximate 3 liters of protein containing material had been loaded, the Q-Sepharose column was disconnected and the CM-Spherodex column was washed with buffer until the A280 absorbance of the eluate was the same as buffer alone. The protein was eluted from the column in 20 mM MES, 400 mM NaCl, pH 5.5 in a volume of approximately 180 ml. This eluted fraction was then reloaded on to a smaller (2 ml) CM-Spherodex column for protein concentration, and the bound protein removed in a single step of 20 mM MES, 1.2 M NaCl, pH 5.5 in a volume of approximately 12 mL. The protein was then loaded directly on to a 150 ml pyrogen-free Sephacryl S-100 column pre-equilibrated with 20 mM sodium citrate, 150 mM NaCl, pH 5.0 buffer. Column fractions, were analyzed by Coommassie-stained (0.5% Coommassie Brilliant Blue-R, 25% isopropanol, 10% methanol, 10% acetic acid) SDS-PAGE and Western analysis. Western analysis was performed using a 1:1000 dilution of a rabbit anti-human BPI antisera. Fractions which contained the nonglycosylated rBPI protein were pooled and resulted in a final lot which was greater than 95% pure as analyzed by Coommassie-stained SDS-PAGE.

The protein samples thus prepared and purified were filtered and/or concentrated for crystallization studies of the purified nonglycosylated rBPI protein. Protein samples were optionally filtered using a 0.2 μm syringe filter (Millipore Corp., Bedford, Mass.) or a 0.2 μm Nalgene filter (Nalge Corp., Rochester, N.Y.) to remove precipitate. Protein samples were concentrated in a Centricon 10 (Amicon Corp., Beverly, Mass.) or a Centriprep 10 (Amicon Corp., Beverly, Mass.). For the Centricon 10 concentrators, a JA 20 rotor (Beckman, Fullerton, Calif.) in a J2-21 Beckman centrifuge was used at 6000 rpm for 60 minutes. For the Centriprep 10 concentrators, a swinging bucket rotor in a J-6B Beckman centrifuge was used at 3000 rpm for 60 minutes. Final volumes for various protein samples prepared for crystallization studies described herein ranged from about 0.1 to 1 mL, and the protein concentrations were generally between about 10 and 20 mg/mL. Protein solutions may be diluted or concentrated for crystallization studies, for example, from about 5 to about 50 mg/ml.

### EXAMPLE 2

### Structure Determination of a Crystallized BPI Protein

Presented herein is the crystal structure of BPI and two bound phospholipids at 2.4 Å resolution. Our model provides the first structural information on the LPS-binding and lipid transport protein family and suggests a common mode of lipid binding for its members.

Purified, full-length, non-glycosylated, recombinant human BPI expressed in CHO cells was crystallized by hanging-drop vapor diffusion at room temperature. The protein concentration was 8.5 mg/ml and the crystallization buffer contained 12% (w/v) PEG 8000, 200 mM magnesium acetate, and 100 mM sodium cacodylate, pH 6.8. Two crystal forms with slightly different cell dimensions grew under the same conditions in space group C₂, with one molecule per asymmetric unit. Form 1 crystals were reproducible and had cell dimensions of a=185.0, b=37.2, c=84.3 Å, and β=101.3°. Form 2 crystals appeared rarely and had cell dimensions of a 185.6, b=33.0, c=85.2 Å, and β=101.6°

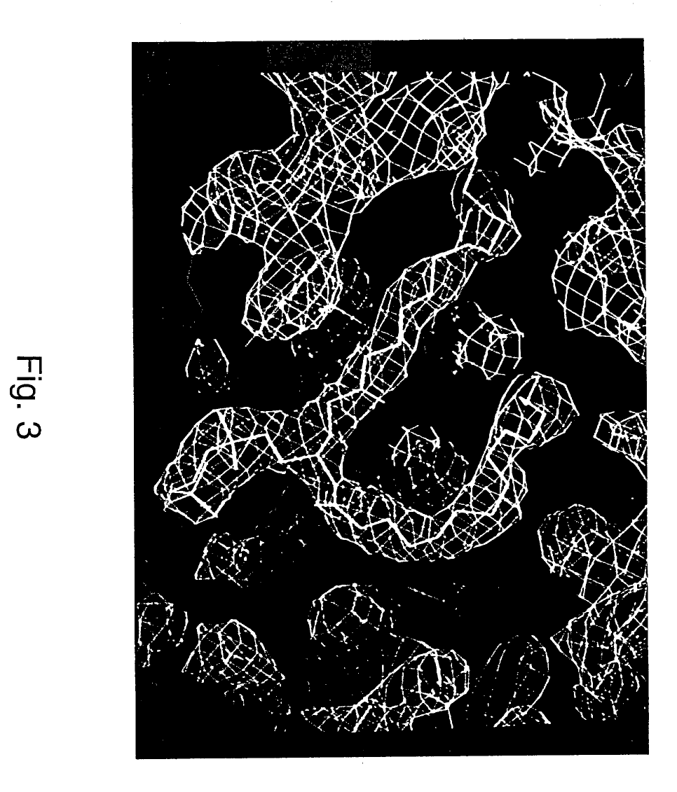

For Table 1, x-ray diffraction data were collected at room temperature with the R-AXIS IIC imaging plate area detector mounted on a Rigaku RU200 rotating anode x-ray generator. Data were processed with DENZO and SCALEPACK [Z. Otwinski, in Proceedings of CCP4 Study Weekend: Data Collection and Processing, L. Sawyer, N. Isaacs, S. Baileys, Eds. (SERC Daresbury Laboratory, Warrington, UK, 1993), pp. 56]. For form 1 crystals, a native data set to 2.8 Å was collected from a single crystal, which was 92.4% complete overall (84.9% complete with an average I/σ(I)=2.3 in the outermost resolution shell). A native data set to 2.4 Å for form 2 was collected from two crystals and was 92.7% complete overall (94.6% complete with an average I/σ(I)=2.6 in the outermost shell). Because they could be reliably reproduced, form 1 crystals were used for all heavy atom soaks. The structure was solved by multiple isomorphous replacement (MIR) with anomalous scattering. Heavy atom sites were identified by difference-Patterson and difference-Fourier maps. Phase refinement was performed with [Collaborative Computational Project No. 4, Acta Crystallogr., D50:760 (1994)] producing a mean figure of merit (FOM) of 0.57. The MIR map (FIG. 3) was improved by density modification including solvent flattening, histogram matching, and phase extension using DM [Collaborative Computational Project No. 4, supra]. After a partial model was obtained with FRODO [T. A. Jones, J. Appl. Crystallogr., 11:268 (1978)], phase combination was performed with SIGMAA [Collaborative Computational Project No. 4, supra] (final FOM=0.89). CMNP is chloro-Hg-nitrophenol; DMM is dimethyl mercury; PCMBS is parachloromercury-benzene sulfonate; TELA is triethyl lead acetate.

TABLE 1
X-RAY DIFFRACTION DATA

| | | Data | | | | | |
|---|---|---|---|---|---|---|---|
| | Resolution | Complete- | Rsym* | Sites | R | Phasing | |
| Item | (Å) | ness | (%) | (N) | Cullis† | Power | Mid‡ |
| Native 1 | 2.8 | 92.4 | 8.6 | | | | |
| Native 2 | 2.4 | 92.7 | 7.2 | | | | |
| CMNP§ | 3.2 | 84.8 | 6.1 | 1 | 66.0 | 2.04 | 0.15 |
| DMM∥ | 3.5 | 72.8 | 9.8 | 11 | 65.0 | 1.49 | 0.26 |
| PCMBS¶ | 3.1 | 66.4 | 9.4 | 3 | 77.0 | 1.27 | 0.38 |
| HgCl₂ | 3.0 | 86.5 | 6.9 | 1 | 49.0 | 2.13 | 0.18 |
| K₂PtCl₄ | 3.2 | 93.3 | 8.2 | 3 | 90.0 | 0.68 | 0.13 |
| K₂PtBr₆ | 3.1 | 94.8 | 5.8 | 3 | 73.0 | 0.88 | 0.14 |
| TELA# | 3.3 | 94.0 | 11.3 | 2 | 86.0 | 0.80 | 0.15 |
| TELA-HgCl₂ | 3.3 | 91.4 | 9.6 | 3 | 63.0 | 1.90 | 0.18 |
| Xenon | 3.4 | 98.2 | 18.9 | 5 | 87.0 | 0.69 | 0.18 |
| K₃UO₂F₅ | 3.0 | 75.0 | 8.6 | 2 | 65.0 | 1.40 | 0.16 |

*Rsym = 100 (Σₕ | |ₕ − < | > |)/(Σh |h) where < | > is the mean intensity of all symmetry-related reflections |h.
†R_{Cullis}= (Σ|F_{PH} +/− F_{P} | − F_{H(calc)})/(Σ|F_{PH} +/− F_{P} |) for centric reflections, Phasing power = [Σ| F_{H(calc)}²|/ (Σ |F_{PH(obs)} − F_{PH(calc)} |²]^{1/2.}
‡MID (mean isomorphous difference) = Σ | F_{PH} − F_{P}|/Σ F_{P}, where F_{PH} is the derivative structure factor and F_{P} is the native structure factor and the sum is over all reflections common to both data sets.
§CMNP, chloro-Hg-nitrophenol;
∥DMM, dimethyl mercury;
¶PCMBS, parachloromercurybenzene sulfonate;
#TELA, triethyl lead acetate

Table 2 relates to model refinement and statistics. The model was refined at 2.8 Å through iterative cycles of simulated-annealing with X-PLOR [A. T. Brünger and A. Krukowski, Acta Crystallogr., A46:585 (1990)] and manual rebuilding. 10% of the data were set aside before refinement began for R_{free} [A. T. Brünger Nature, 355:472 (1992)] calculations. When the model had been refined to an R-factor* of 20.4% (R_{free}=32.6%) with the 2.8 Å data, rigid-body minimization was performed against the 2.4 Å data set (R=29.8% to 3.5 Å after minimization). Additional cycles of simulated annealing, positional refinement, correlated individual temperature factor refinement, and manual rebuilding reduced the R-factor to 22.7% and R_{free}=31.3% (no intensity cutoff). An overall anisotropic temperature factor and bulk solvent correction were applied to the observed reflections when R_{free} showed improvement. The model was confirmed by calculating simulated-annealing omit-maps for every part of the structure. The final model contains all 456 residues of the protein, 48 well-ordered waters, and 2 molecules of phosphatidylcholine. Regions of the backbone with poor electron density include residues 148, 232-236, 258-260, and parts of the loop between residues 281-311. Sidechains with poorly defined density were truncated to alanine. The model was examined by the programs PROCHECK [R. A. Laskowski, M. W. McArthur, D. S. Moss, J. M. Thornton, J. Appl. Crystallogr. 26:283 (1993)], VERIFY [R. Lüthy, J. U. Bowie, D. Eisenberg, Nature, 356:83 (1992)], and ERRAT [C. Colovos and T. Yeates, Protein Sci., 2:1511 (1993)].

TABLE 2
REFINEMENT STATISTICS FOR FORM 2 CRYSTALS

| Data | |
|---|---|
| Resolution (Å) | 2.4 |
| Unique reflections (N) | 18,898 |
| Completeness (%) | 92.7 |
| Atoms in model | |
| Protein (non-hydrogen) | 3532 |
| Phosphatidylcholine | 102 |
| Water | 48 |
| Refinement parameters | |
| Resolution range (Å) | 50.0-2.4 |
| R-factor* (%) | 22.7 |
| R_{free}(%) | 31.3 |
| Avg. atomic B factors | |
| Protein | 36.9 |
| Lipid N, C | 49.4, 51.0† |
| Waters | 44.6 |
| rms‡ deviation from ideality | |
| Bonds (Å) | 0.006 |
| Angles (deg) | 1.4 |
| Dihedrals (deg) | 26.0 |
| Impropers (deg) | 1.2 |

*R = 100 (Σ | F_{obs} − F_{c} |) / (Σ F_{obs}) where F_{obs} and F_{c} are the observed and calculated structure factors, respectively.
†B factors for the lipids bound in the NH₂— and COOH-terminal domains.
‡rms = root mean square

BPI is a boomerang-shaped molecule with approximate dimensions of 135 by 35 by 35 Å (FIGS. 1, A and B). It consists of two domains of similar size (NH₂- and COOH-terminal) that are connected by a proline-rich linker of 21 residues (positions 230 to 250). The two domains form three structural units; barrels are found at each end of the protein, and a central β sheet forms an interface between the barrels. The secondary structure and topology of the two domains are similar, giving the protein pseudo-twofold symmetry.

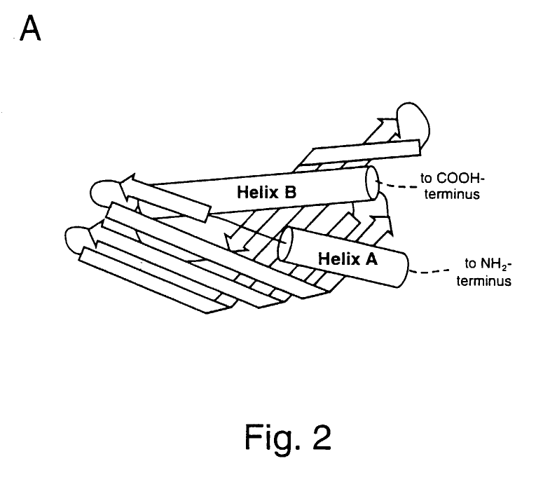

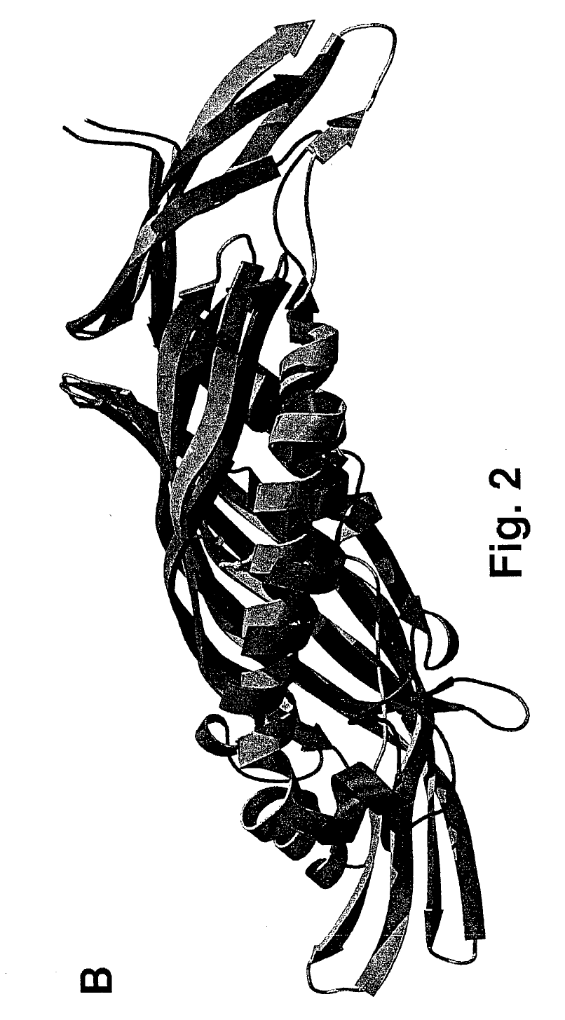

Each barrel (residues 10 to 193 and 260 to 421) contains three common structural elements: a short α helix, a five-stranded antiparallel β sheet, and a long helix (FIG. 2A), in that order. We call these elements helix A, sheet N and helix B in the NH₂-terminal domain, and helix A′, sheet C and helix B′ in the COOH-terminal domain. Sheets N and C have a series of β bulges that change the direction of their strands and cause a pronounced curve in the sheets. In each domain, the long helix lies along the concave face of the sheet, with the helical axis at ˜60° to the strands of the β sheet. A single disulfide bond between Cys¹³⁵ and Cys¹⁷⁵ anchors helix B to the final strand of sheet N. Situated between the NH₂- and COOH-terminal barrels is a twisted, seven-stranded antiparallel β sheet composed of four strands from the NH₂-terminal domain and three strands from the COOH-terminal domain. This central sheet forms an interface between the two domains and is thus reminiscent of several dimer interfaces stabilized by hydrogen bonds between strands of a β sheet [M. Leeson, B. Henderson, J. Gillig, J. Schwab and J. Smith, Structure, 4:253 (1996); D. Ohlendorf, W. F. Anderson, M. Lewis, C. O. Pabo, B. W. Matthews, J. Mol. Biol., 169:757 (1983); G. N. Reeke, J. W. Becker, G. M. Edelman, J. Biol. Chem., 250:1525 (1975)].

The structural similarity of the two domains of BPI is shown by the superposition [G. H. Cohen, J. Mol. Biol., 190:593 (1986)] in FIG. 2B; they are related by a rotation of 173° and have a root mean square deviation (rmsd) of 3.0 Å on the basis of superposition of 169 Cα pairs. The structure shared by these two domains does not resemble other protein folds; several structural alignment programs [N. N. Alexandrov and D. Fischer, Proteins, 25:354 (1996); D. Fischer, C. J. Tsai, R. Nussinov, Protein Eng., 8:981 (1995); L. Holm, C. Sander, Nucl. Acids Res., 22:3600 (1996)] failed to reveal a significant match to any known folds. Significant differences between the superimposed domains are found in two loop regions containing residues 45 and 96 in the NH₂-terminal domain and residues 280 and 348 in the COOH-terminal domain. These differences may be functionally important because the loops around residues 45 and 96 in the NH₂-terminal domain have been implicated in LPS binding and bactericidal activity (see below). This structural similarity of the two domains was unexpected, not only because of their lack of significant sequence identity (<20%), but also because of their functional differences. The NH₂-terminal domain of BPI is cationic and retains the bactericidal, LPS-binding, and LPS-neutralization activities of the intact protein [A. H. Horwitz, et al., Protein Expr. and Purif., 8:28 (1996); C. E. Ooi, J. Weiss, P. Elsbach, B. Frangione and B. Mannion, J. Biol. Chem., 262:14891-14894 (1987); C. E. Ooi, J. Weiss, M. E. Doerfler and P. Elsbach, J. Exp. Med., 174:649 (1991)]. The COOH-terminal domain is essentially neutral and shows limited LPS-neutralization activity,[S. L. Abrahamson et al., J. Biol. Chem., 272:2149 (1997)]. However, the structural similarity of the two domains may reflect a previously undetected functional similarity: each domain contains a binding pocket for a phospholipid.

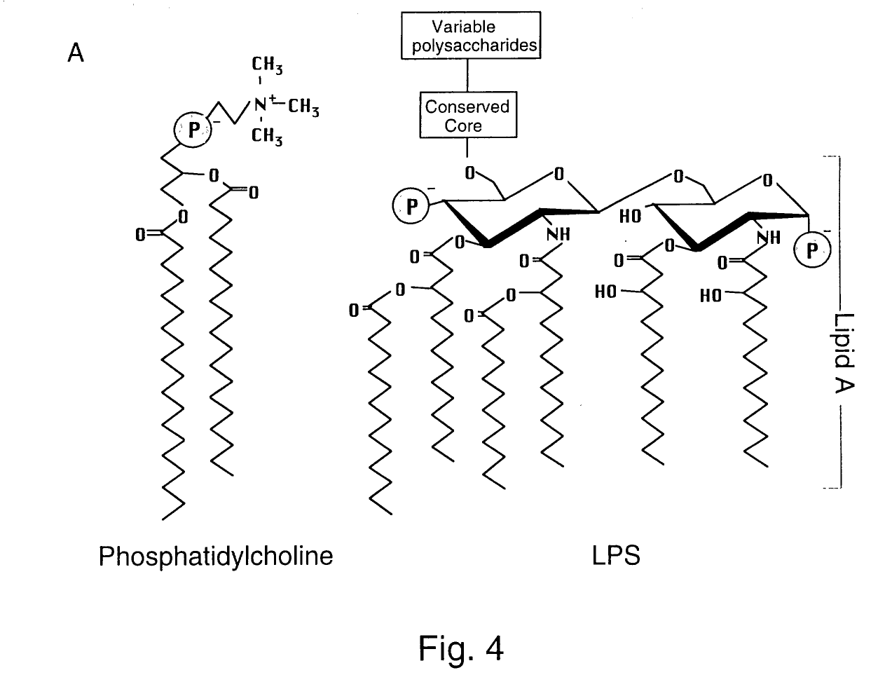

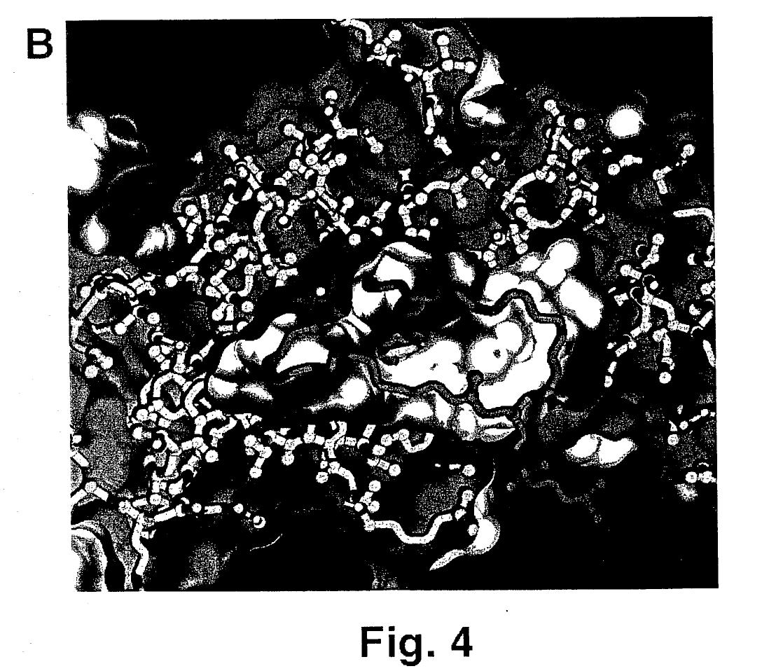

After the amino acid sequence had been traced in the electron density maps, two regions of extended electron density remained that could not be accounted for by protein atoms. This density, found in the interior of both domains, was present in the multiple isomorphous replacement (MIR) maps (FIG. 3) at an intensity similar to that of the protein density, and it became the predominant feature in F_{obs}-F_{calc} maps after sequence fitting (both form 1 and form 2 crystals). Electrospray mass spectrometry of the sample used for crystallization revealed two molecules, with relative molecular masses of 522 and 787, in approximately equal amounts. Tandem mass spectrometric analysis was consistent with the two species being phosphoglycerides containing a phosphatidylcholine head group and either one or two 18-carbon acyl chains with one double bond. Phosphatidylcholine (FIG. 4A) is abundant in eukaryotic cells and is presumably bound by BPI in the cells from which the protein is isolated.

The two lipids are bound in extensive apolar pockets on the concave surface of the boomerang, situated between the NH₂-terminal and COOH-terminal barrels and the central β sheet. In the NH₂-terminal domain, the entrance to the pocket is formed by helices A and B. The back and sides are formed by sheet N and the central sheet. The two acyl chains insert ˜15 Å into the interior of the protein and are surrounded by apolar side chains (FIG. 4B). The head group lies at the entrance of the pocket and is exposed to solvent. The pocket in the COOH-terminal domain, which has a slightly larger opening, is formed by the analogous secondary structures. Both basic and acidic side chains found near the entrances of the pockets are available for electrostatic interactions with the zwitterionic head group. When the lipids are removed from the model, the pocket in NH₂-terminal domain has a solvent accessible surface area [M. L. Connolly, Science, 221:709 (1983); M. L. Connolly, J. Am. Chem. Soc., 107:1118 (1985)] of 557 Å², and the pocket in the COOH-terminal domain has an area of 413 Å², for a total of 970 Å². The intensity of the electron density for the two acyl chains in both pockets is similar and does not indicate whether the single acyl chain species is found predominantly in either pocket.

The discovery of bound phospholipid in our structure suggests a possible site of interaction between BPI and LPS. As seen in FIG. 4A, phosphatidylcholine and LPS share some structural similarity, including negatively charged phosphate groups and, most notably, acyl chains. Since BPI's function is to bind a lipid, LPS, and since lipid is bound in pockets of BPI, it seems reasonable that the acyl chains of LPS bind in the apolar pockets. The following observations support his hypothesis: i) the acyl chains of lipid A are known to be essential for binding by BPI [H. Gazzano-Santoro et al., Infection and Immunity, 63:2201 (1995)]; ii) the binding pockets of BPI are reminiscent of cavities in other lipid-binding proteins [L. Banaszak et al., Adv. Protein Chem., 45:89 (1994)]; and iii) BPI has a significant sequence similarity to two lipid transfer proteins (see below).

Our proposed site of interaction between BPI and the acyl chains of LPS differs from that suggested by previous work focusing on the NH₂-terminal domain. Fragments containing the NH₂-terminal domain of BPI have been identified with equivalent or greater bactericidal and LPS-binding activities relative to the full-length protein [A. H. Horwitz, et al., Protein Expression and Purification, 8:28 (1996); C. E. Ooi, J. Weiss, M. E. Doerfler and P. Elsbach, J. Exp. Med., 174:649 (1991)]. The activity of one NH₂-terminal fragment was reduced when residues past positions 12 or between positions 169 and 199 were deleted [C. Capodici and J. Weiss, J. Immunol., 156:4789 (1996)]. The structure shows that these deletions affect elements of the barrel (at the beginning of helix A and from the middle to the end of helix B) and could significantly alter its structure. While the barrel seems to be the minimal structural unit with full activity, three smaller regions of this domain retain significant LPS-binding, LPS-neutralization, and bactericidal activity [R. G. Little, D. N. Kelner, E. Lim, D. J. Burke and P. J. Conlon, J. Biol. Chem., 268:1865 (1994)]: residues 17-45 (most of helix A and the first β strand of sheet N), residues 82-108 (a β hairpin [residues 82 to 106 of-BPI show limited sequence similarity with residues 32 to 51 of the limulus anti-LPS factor (LALF), and have been predicted to form an amphipathic β hairpin similar to that seen in the LALF structure [A. Hoess, S. Watson, G. R. Silber and R. Liddington, EMBO 12:3351 (1993)]. Although this region of BPI does form β hairpin, the strict amphipathic character of the loop seen in LALF is not maintained, and a structural superposition shows that the sequence of BPI must be shifted by one residue relative to the proposed sequence alignment] between strands 3 and 4 of sheet N), and residues 142-169 (a segment preceding and including part of helix B). These three regions include 18 basic residues (and only 4 acidic residues) and form a positively charged tip on the NH₂-terminal domain (on the left of FIG. 1) which may make favorable electrostatic interactions with negatively charged groups of LPS. Further studies are necessary to determine the relative importance of the apolar pockets and positively charged NH₂-terminal tip to BPI's LPS-binding and bactericidal activities.

BPI is the first member of the mammalian LPS-binding and lipid transfer family to have its three-dimensional structure determined. BPI and LBP are related to two lipid transfer proteins, cholesteryl ester transfer protein (CETP) and phospholipid transfer protein (PLTP) [A. Tall, Annu. Rev. Biochem., 64:235 (1995)]. Alignment of the amino acid sequences of human BPI, LBP, CETP, and PLTP with BPI's secondary structure (FIGS. 5, A and B) shows that structurally important residues are conserved in the four proteins. The two cysteines that form the single disulfide bond and are critical to the function of BPI [A. H. Horwitz, et al., Protein Expression and Purification, 8:28 (1996)] are completely conserved. Also, the pattern of hydrophobic/hydrophilic residues in the β strands indicates that the β bulges responsible for the extensive sheet twisting are preserved. The conserved sequences strongly suggest that members of the LPS-binding and lipid transfer family share BPI's two-domain structure and that the two domains are similar in topology.

It is likely that the lipid transfer proteins will also share the apolar binding pockets found in BPI. Striking parallels are found between our BPI-phosphatidylcholine structure and previous work showing that CETP copurifies with an equimolar amount of phosphatidylcholine [A. Tall, Annu. Rev. Biochem., 64:235 (1995)] and has two distinct binding sites [S. Wang, L. Deng, R. W. Milne and A. R. Tall, J. Biol. Chem., 267:17487 (1992)]—one for neutral lipids and another for phospholipids. The known ligands of CETP and PLTP (cholesteryl esters, triglycerides, retinyl esters and phospholipids) all contain at least one acyl chain which could bind in apolar pockets similar to those in BPI, suggesting a common mode of ligand binding in this family. Sequestration of these hydrophobic chains in interior pockets may be critical to the function of the lipid transfer proteins: transfer of apolar ligands in an aqueous environment. Thus, the structure of BPI illuminates the action of the plasma lipid transfer proteins, as well as offering possibilities for how BPI and LBP interact with LPS.

### EXAMPLE 3

### Molecular Modeling of BPI Ligands and Mimetics

We have used the information derived from the X-ray crystal structure of BPI presented herein, along with the teachings of the art, including, for example, WO94/20532 (PCT/US94/02465) to design various BPI-related proteins and peptides. These constructs may be divided into categories as illustrated below, including peptides and proteins, including fragments, analogs and variants of the protein, since they best describe the different ways in which different domains and portions may be assembled to achieve new molecules.

1. Individual Peptide Domains

The overlapping BPI peptide data indicated that the N-terminal domain of BPI contains at least three independent functional domains that have one or more of the biological activities of BPI, including, for example, antibacterial, antifungal, anti-heparin and anti-angiogenic activities. Domain I is a region of amino acid residues from about 17 to about 45; Domain II is a region of amino acid residues from about 65 to about 99; and Domain III is a region of amino acid residues from about 142 to about 169. Hundreds of peptide sequences derived from these domains have been synthesized, including addition, deletion and substitution variants of the domain-derived sequences. Through further refinements, smaller “core” regions within these domains have been identified that still retain high levels of activity; for example, within Domain II residues 90-99 and within Domain III residues 148-161.

These peptides have included linear molecules that may or may not assume a conformation that maximally express activity. From the X-ray structure data, segments of BPI are designed that should preserve the three-dimensional structure of these domains when constructed outside the context of the intact protein. For example, both Domains I and II contain hairpin loop structures that are positioned adjacent to one another in space on the proximal tip of the molecule. In contrast, although Domain III is a helix+turn and not a loop, extensions from both ends of the domain are positioned near enough to each other to consider linking them together. As a result, peptides can be designed that reflect these structures by replacing selected residues in synthetic or recombinant peptides with cysteines, so as to create disulfide-stablized domain mimetics. Since this approach is based upon the actual structure of BPI, it differs from those of other groups that are based upon putative alignments between BPI and the structure of proteins such as the Limulus amoebocyte lysate factor (LALF). As examples of these embodiments, listed below are a series of exemplary peptides that, with cysteines added to the N- and C-terminus could assume structures similar to those seen in the intact protein: from Domain I: residues 36-54; from Domain II: residues 84-109, 85-108, 86-107, 87-106, 88-105, 89-104, or 90-103; and from Domain III: residues 142-164.

2. Peptide Domain Hybrids

It has also been demonstrated that certain peptide domain hybrids, which include repeats of the same sequence from a single domain or inter-domain combination of sequences, have enhanced activities. For example, linking Domain II- and III-derived peptides (such as XMP.29: 85-99::148-161) has enhanced biological activity. Interestingly, these domains in the crystal structure are closely associated in space, and peptide XMP.29 may represent a Domain II::III hybrid that actually shares some structural similarity to the intact protein. Based upon the X-ray structure, a Domain II-III peptide that consists of approximately residues 90-103::146-162 is constructed. Such a peptide may even more closely mimic what is seen in the protein.

3. BPI “Tip” Mimetics

As discussed above, portions of all three peptide domains discovered and described in WO94/20532 come together on the proximal tip of the N-terminal fragment. As a result, a BPI “tip” mimetic is designed that essentially “slices” off the most extreme tip of the molecule but preserves the critical domain elements. Such a slice would lack the hydrophobic pockets found in the intact protein, but may exhibit activities beyond those of the individual segments. The following segments represent such a “slice” of the three peptide domains. However, to best preserve the geometry between the domains, it would be desirable to insert “linker” sequences between them so as to ensure proper positioning. By fixing these segments in space, programs such as InsightII (Molecular Simulations, Inc.) can identify possible linker sequences by i) searching protein databases for similar structures or ii) de novo designing appropriate linkers. In this regard it may be desirable to utilize residues that are not readily susceptible to proteolysis (Ala, Ser, Gly, etc.), or to utilize amino acids like Pro that impose additional spatial constraints on peptide structure. An exemplary peptide consists of Domain I-II-III-derived elements: 37-54:90-104:144-162.

Similarly, by analogy with the above-described cyclic domain structures, the fact that residues 37 and 162 are positioned near each other in the protein suggests that a cyclic tip mimetic could be created by replacing these residues with cysteines, for example, Cys::38-54:90-104:144-161::Cys.

4. Extended N-terminal Domains of BPI

The three dimensional structure of BPI indicates that the molecule, which forms N- and C-terminal domains, can be divided into three structural units as described in Example 2. Two of these units represent the N- and C-terminal “barrels” that are formed by residues 10-193 and 260-421, respectively, whereas the third element is a central β sheet structure that forms the interface between the two barrels. Of interest is the fact that the two bound phospholipids in BPI occupy spaces between the two barrel structures and the central β sheet. As a result, the recombinant BPI protein product rBPI₂₁, which essentially contains residues 1-193 lacks some of the structural components necessary to form a complete hydrophobic pocket. A new molecule is constructed that encodes residues 1 to approximately 260 which would contain most of the residues necessary to form a complete hydrophobic pocket.

5. Mutants for Immobilizing rBPI₂₁

One application for BPI protein products is their use as affinity removal ligands for endotoxin in solution. For example, immobilizing a BPI protein on a column or membrane matrix would allow removal of endotoxin from endotoxin-contaminated solutions by simply passing those solutions over the immobilized BPI protein. Some of the cysteine-mutated peptides described above may be useful for this purpose, as well as rBPI₂₃. Alternatively, in order to selectively couple a stable, readily produced protein like rBPI₂₁ to a column or membrane, a cysteine could be added to the N- or C-terminus, thus allowing site-specific conjugation and selective orientation of the binding “tip” away from the solid support. Such a construct is alternatively constructed by adding a short linker segment (such as Gly-Gly-Gly-Ser) to the C-terminal residue of the BPI protein product, for example, residue 193 of rBPI(1-193), followed by a cysteine residue. Such a construct would have a high probability of folding correctly, given the domain nature of residues 1-193, and be readily conjugatable. Similarly, a series of new conjugates between rBPI(1-193)C and other thiol-containing proteins or molecules is created for the purposes of evaluating new molecules.

6. New N-terminal Dimeric Molecules

As an extension of the above analysis, a new series of N-terminal dimeric molecules can be constructed that take better advantage of the hydrophobic pockets. For example, by replacing the C-terminal barrel with another copy of the N-terminal barrel, an analog of BPI would be created that contains two functional barrels and possibly two functional hydrophobic pockets. One such dimer could be constructed by replacing residues 260-456 with residues 1-193. Alternatively, other more central locations may be identified within the β sheet structure where symmetry would dictate additional and even better points for duplication.

7. C-terminal Fusion Proteins

The C-terminal domains of LBP and CETP appear to mediate interactions with CD14 and lipoproteins. Similarly, the C-terminal domain of BPI has LPS binding and neutralization activities. As a consequence, the C-terminal barrel of BPI (or other family members) could be fused to barrels or domains of other family members and/or to other proteins to alter/modify/enhance their action.

8. Homology Modeling of BPI Family Members

The BPI coordinates have been useful in the generation of molecular models of other members of the BPI protein family. Utilizing CLUSTAL (a multiple sequence alignment program in PC-Gene) and the Homology module (a structure-based homology modeling program in InsightII on the SGI), molecular models (and the corresponding three-dimensional coordinates files) of lipopolysaccharide binding protein (LBP), cholesteryl ester transfer protein (CETP) and phospholipid transfer protein (PLTP) have been generated. With these files, existing mutants are mapped and new ones designed. Published data [Wang et al., Biochemistry 30:3484-3490, (1991)] indicate that insertional alterations in three locations of CETP severely impaired cholesteryl ester transfer activity: residues 48-53, residue 165, and residues 373-379. Since residues 48-53 and residue 165 of CETP coincide structurally with Domains I and III of BPI respectively, the functional domain structure of BPI extends to the other protein family members. Similarly, by virtue of the symmetry between the N- and C-terminal domains, it is likely that the corresponding residues on the C-terminal tip of BPI are involved in recognizing receptors and/or interacting with lipoproteins.

9. Lipid Pocket Mutants

A detailed compilation of the residues in BPI which form the pockets is described in Table 3 as follows. Column 1 of Table 3 indicates the residue name and number. Column 2 shows checked residues which indicate the residues that show a change in solvent accessible surface area with lipid binding. Column 3 shows checked residues that indicate the residues that have some atom within 4 A of a lipid atom. If the contact is to the head-group of the lipid, the residues are listed at the end, under ENTRY ONLY. Column 4 indicates conservation in 3 BPI and 4 LBP sequences, e.g., for Ile 68, in 3 of the 7 sequences, the residue is similarly Ile; for the other 4 sequences, the residue is Leu (see also column 5). Column 5 indicates alternative residues which occur in BPI or LBP at that position for the 7 BPI and LBP sequences analyzed. Column 6 indicates residues for mutations to block the pockets, using residues selected to be well-conserved (especially in the N-terminal domain) and relatively small. The suggested mutations are all to large sidechains in order to decrease the size of the pocket by as much as possible.

TABLE 3
N-TERMINAL POCKET - residues contributing to interior

| | | Within | Conserva- | Alterna- | Muta- |
|---|---|---|---|---|---|
| Residue¹ | ΔSA² | 4¶³ | tion⁴ | tives⁵ | tions⁶ |
| Val 7 | ✓ | ✓ | 2/7 | A, T | |
| Ile 9 | ✓ | ✓ | All | | W |
| Gly 13 | ✓ | ✓ | All | | |
| Leu 14 | ✓ | | All | | |
| Tyr 16 | ✓ | | All | | |
| Ala 17 | ✓ | ✓ | All | | F |
| Ser 18 | ✓ | ✓ | 1/7 | C, A | |
| Gly 21 | ✓ | ✓ | All | | F |
| Ala 24 | ✓ | ✓ | 4/7 | V, T, S | Y, H |
| Leu 25 | ✓ | | All | | |
| Ile 68 | ✓ | ✓ | 3/7 | L | |
| Leu 76 | ✓ | ✓ | All | | |
| Phe 78 | ✓ | ✓ | 1/7 | V, L | |
| Leu 117 | ✓ | ✓ | All | | |
| Leu 119 | ✓ | | All | | |
| Pro 128 | ✓ | | 5/7 | A, S | |
| Ile 130 | ✓ | | 1/7 | V | |
| Val 178 | ✓ | | 2/7 | L, I | |
| Val 182 | ✓ | | All | | |
| Glu 185 | ✓ | ✓ | 1/7 (allele) | K, D, H | |
| Leu 186 | ✓ | ✓ | All | | W |
| Tyr 189 | ✓ | ✓ | All | | |
| Phe 190 | ✓ | ✓ | 2/7 | V, L | |
| Leu 193 | ✓ | ✓ | All | | |
| Leu 220 | ✓ | ✓ | All | | |
| Val 222 | ✓ | | 5/7 | M, W | |
| Val 254 | ✓ | ✓ | 6/7 | I | |
| Leu 256 | ✓ | | 3/7 | F | |
| Pro 428 | ✓ | ✓ | All | | |
| Thr 429 | ✓ | ✓ | 1/7 | M, L | |
| Pro 430 | ✓ | ✓ | 5/7 | L | |
| Val 433 | ✓ | | 3/7 | I | |
| Leu 435 | ✓ | | All | | |
| Val 453 | ✓ | | 6/7 | I | |

ENTRY ONLY

| | | | | |
|---|---|---|---|---|
| Gln 20 | ✓ | ✓ | 3/7 | E |
| Lys 27 | ✓ | ✓ | 3/7 | R, S |
| Glu 28 | ✓ | | 6/7 | K |
| Arg 31 | ✓ | | 2/7 | K, E |
| Ser 181 | ✓ | ✓ | 5/7 | T, A |
| Arg 432 | ✓ | ✓ | 3/7 | K, Y, H |
| Tyr 455 | ✓ | ✓ | 6/7 | H |

C-TERMINAL POCKET - residues contributing to interior

| | | | | | |
|---|---|---|---|---|---|
| Phe 263 | ✓ | ✓ | All | | |
| Asn 264 | ✓ | | All | | |
| Ala 266 | ✓ | ✓ | All | | F |
| Gly 267 | ✓ | ✓ | 2/7 | A, S, T, | |
| Val 275 | ✓ | ✓ | 1/7 | A, Y | |
| Leu 276 | ✓ | ✓ | 5/7 | F, W | F, W |
| Lys 277 | ✓ | | 1/7 | G, N | |
| Met 278 | ✓ | ✓ | 1/7 | L, F | |
| Val 318 | ✓ | | 1/7 | L, I, G | |
| Ala 320 | ✓ | | 2/7 | V | |
| Pro 324 | ✓ | ✓ | 6/7 | Q | |
| Leu 326 | ✓ | ✓ | 6/7 | V | |
| Phe 335 | ✓ | ✓ | 1/7 | L, V, E | |
| Pro 337 | ✓ | ✓ | 5/7 | A, F | |
| Val 339 | ✓ | | 2/7 | L, M | |
| Met 360 | ✓ | ✓ | 2/7 | L, V | |
| Thr 362 | ✓ | ✓ | 5/7 | L | |
| Val 368 | ✓ | ✓ | 2/7 | I, L | |
| Leu 375 | ✓ | ✓ | 3/7 | I, V | |
| Val 376 | ✓ | ✓ | 2/7 | I, T | |
| Gly 377 | ✓ | | All | | |
| Leu 379 | ✓ | ✓ | All | | |
| Leu 381 | ✓ | ✓ | 3/7 | P | |
| Val 409 | ✓ | ✓ | 1/7 | L, M, I | |
| Val 413 | ✓ | ✓ | 1/7 | F, L | F |
| Val 417 | ✓ | ✓ | 3/7 | I, F | W |
| Lys 420 | ✓ | ✓ | 5/7 | E | Y, H |
| Leu 421 | ✓ | ✓ | 5/7 | I, F | |
| Phe 425 | ✓ | ✓ | 6/7 | L | |

ENTRY ONLY

| | | | | |
|---|---|---|---|---|
| Asp 200 | ✓ | | All | |
| Ser 201 | ✓ | ✓ | 4/7 | K, T, N |
| Val 202 | ✓ | | 4/7 | F, I |
| Tyr 270 | ✓ | ✓ | All | |
| Arg 416 | ✓ | ✓ | 1/7 | K, V, D |
| Lys 423 | ✓ | | 3/7 | R, E, Q |

¹Residue name and number.
²Checked residues show a change in solvent accessible surface area with lipid binding.
³Checked residues have some atom within 4 A of a lipid atom (if the contact is to the head group of the lipid, the residues are listed at the end, under ENTRY ONLY).
⁴Conservation in 3 BPI and 4 LBP sequences, e.g., for Ile 68, in 3 of the 7 sequences, the residue is similarly Ile; for the other 4 sequences, the residue is Leu (see note 5).
⁵Indicates alternative residues which occur in BPI or LBP at that position for the 7 BPI and LBP sequences analyzed.
⁶Indicates residues for mutations to block the pockets using residues selected to be well-conserved (especially in the N-terminal domain) and relatively small. The suggested mutations are all to large sidechains in order to decrease the size of the pocket by as much as possible.

TABLE 4

| | | | | | | | | | | |
|---|---|---|---|---|---|---|---|---|---|---|
| ATOM | 1 | CB | VAL | A | 1 | 95.942 | 11.564 | 18.390 | 1.00 | 51.23 |
| ATOM | 2 | CG1 | VAL | A | 1 | 97.217 | 12.313 | 18.805 | 1.00 | 48.90 |
| ATOM | 3 | CG2 | VAL | A | 1 | 94.694 | 12.383 | 18.720 | 1.00 | 50.67 |
| ATOM | 4 | C | VAL | A | 1 | 96.124 | 12.502 | 16.083 | 1.00 | 47.46 |
| ATOM | 5 | O | VAL | A | 1 | 97.238 | 12.990 | 15.911 | 1.00 | 49.54 |
| ATOM | 8 | N | VAL | A | 1 | 97.161 | 10.372 | 16.554 | 1.00 | 51.03 |
| ATOM | 10 | CA | VAL | A | 1 | 95.982 | 11.216 | 16.876 | 1.00 | 48.79 |
| ATOM | 11 | N | ASN | A | 2 | 95.020 | 13.016 | 15.554 | 1.00 | 42.80 |
| ATOM | 13 | CA | ASN | A | 2 | 95.067 | 14.266 | 14.810 | 1.00 | 38.18 |
| ATOM | 14 | CB | ASN | A | 2 | 93.709 | 14.566 | 14.185 | 1.00 | 39.01 |
| ATOM | 15 | CG | ASN | A | 2 | 93.494 | 13.831 | 12.892 | 1.00 | 40.06 |
| ATOM | 16 | OD1 | ASN | A | 2 | 94.323 | 13.020 | 12.487 | 1.00 | 42.94 |
| ATOM | 17 | ND2 | ASN | A | 2 | 92.380 | 14.108 | 12.228 | 1.00 | 39.49 |
| ATOM | 20 | C | ASN | A | 2 | 95.439 | 15.376 | 15.786 | 1.00 | 34.97 |
| ATOM | 21 | O | ASN | A | 2 | 94.946 | 15.399 | 16.916 | 1.00 | 37.36 |
| ATOM | 22 | N | PRO | A | 3 | 96.373 | 16.260 | 15.397 | 1.00 | 30.80 |
| ATOM | 23 | CD | PRO | A | 3 | 97.144 | 16.244 | 14.143 | 1.00 | 28.90 |
| ATOM | 24 | CA | PRO | A | 3 | 96.806 | 17.367 | 16.252 | 1.00 | 28.19 |
| ATOM | 25 | CB | PRO | A | 3 | 98.083 | 17.830 | 15.571 | 1.00 | 26.30 |
| ATOM | 26 | CG | PRO | A | 3 | 97.765 | 17.619 | 14.128 | 1.00 | 26.17 |
| ATOM | 27 | C | PRO | A | 3 | 95.765 | 18.482 | 16.283 | 1.00 | 28.00 |
| ATOM | 28 | O | PRO | A | 3 | 95.104 | 18.758 | 15.275 | 1.00 | 28.89 |
| ATOM | 29 | N | GLY | A | 4 | 95.615 | 19.108 | 17.444 | 1.00 | 26.31 |
| ATOM | 31 | CA | GLY | A | 4 | 94.653 | 20.182 | 17.588 | 1.00 | 25.44 |
| ATOM | 32 | C | GLY | A | 4 | 95.178 | 21.508 | 17.091 | 1.00 | 25.34 |
| ATOM | 33 | O | GLY | A | 4 | 94.407 | 22.425 | 16.831 | 1.00 | 28.37 |
| ATOM | 34 | N | VAL | A | 5 | 96.494 | 21.621 | 16.981 | 1.00 | 23.98 |
| ATOM | 36 | CA | VAL | A | 5 | 97.134 | 22.842 | 16.519 | 1.00 | 21.80 |
| ATOM | 37 | CB | VAL | A | 5 | 97.671 | 23.681 | 17.689 | 1.00 | 18.63 |
| ATOM | 38 | CG1 | VAL | A | 5 | 98.573 | 24.785 | 17.171 | 1.00 | 20.04 |
| ATOM | 39 | CG2 | VAL | A | 5 | 96.526 | 24.283 | 18.468 | 1.00 | 20.89 |
| ATOM | 40 | C | VAL | A | 5 | 98.308 | 22.436 | 15.660 | 1.00 | 24.04 |
| ATOM | 41 | O | VAL | A | 5 | 99.014 | 21.486 | 15.984 | 1.00 | 27.65 |
| ATOM | 42 | N | VAL | A | 6 | 98.492 | 23.139 | 14.551 | 1.00 | 25.94 |
| ATOM | 44 | CA | VAL | A | 6 | 99.589 | 22.874 | 13.633 | 1.00 | 26.54 |
| ATOM | 45 | CB | VAL | A | 6 | 99.082 | 22.292 | 12.294 | 1.00 | 29.26 |
| ATOM | 46 | CG1 | VAL | A | 6 | 100.253 | 22.038 | 11.357 | 1.00 | 31.93 |
| ATOM | 47 | CG2 | VAL | A | 6 | 98.318 | 21.001 | 12.535 | 1.00 | 30.52 |
| ATOM | 48 | C | VAL | A | 6 | 100.289 | 24.191 | 13.355 | 1.00 | 24.99 |
| ATOM | 49 | O | VAL | A | 6 | 99.638 | 25.202 | 13.135 | 1.00 | 26.97 |
| ATOM | 50 | N | VAL | A | 7 | 101.611 | 24.183 | 13.416 | 1.00 | 26.91 |
| ATOM | 52 | CA | VAL | A | 7 | 102.404 | 25.371 | 13.156 | 1.00 | 27.18 |
| ATOM | 53 | CB | VAL | A | 7 | 103.401 | 25.637 | 14.298 | 1.00 | 27.59 |
| ATOM | 54 | CG1 | VAL | A | 7 | 104.343 | 26.773 | 13.925 | 1.00 | 27.21 |
| ATOM | 55 | CG2 | VAL | A | 7 | 102.659 | 25.967 | 15.575 | 1.00 | 27.05 |
| ATOM | 56 | C | VAL | A | 7 | 103.190 | 25.103 | 11.889 | 1.00 | 29.61 |
| ATOM | 57 | O | VAL | A | 7 | 103.863 | 24.084 | 11.777 | 1.00 | 32.24 |
| ATOM | 58 | N | ARG | A | 8 | 103.077 | 25.994 | 10.917 | 1.00 | 33.04 |
| ATOM | 60 | CA | ARG | A | 8 | 103.801 | 25.841 | 9.668 | 1.00 | 33.25 |
| ATOM | 61 | CB | ARG | A | 8 | 102.842 | 25.631 | 8.494 | 1.00 | 35.70 |
| ATOM | 62 | CG | ARG | A | 8 | 102.195 | 24.244 | 8.485 | 1.00 | 41.64 |
| ATOM | 63 | CD | ARG | A | 8 | 101.309 | 24.004 | 7.259 | 1.00 | 44.24 |
| ATOM | 64 | NE | ARG | A | 8 | 102.049 | 23.578 | 6.073 | 1.00 | 48.00 |
| ATOM | 66 | CZ | ARG | A | 8 | 101.565 | 23.626 | 4.835 | 1.00 | 51.86 |
| ATOM | 67 | NH1 | ARG | A | 8 | 100.341 | 24.091 | 4.611 | 1.00 | 52.72 |
| ATOM | 70 | NH2 | ARG | A | 8 | 102.293 | 23.177 | 3.820 | 1.00 | 53.10 |
| ATOM | 73 | C | ARG | A | 8 | 104.682 | 27.052 | 9.441 | 1.00 | 32.06 |
| ATOM | 74 | O | ARG | A | 8 | 104.196 | 28.168 | 9.288 | 1.00 | 32.62 |
| ATOM | 75 | N | ILE | A | 9 | 105.986 | 26.830 | 9.515 | 1.00 | 31.21 |
| ATOM | 77 | CA | ILE | A | 9 | 106.955 | 27.888 | 9.315 | 1.00 | 31.97 |
| ATOM | 78 | CB | ILE | A | 9 | 108.210 | 27.633 | 10.167 | 1.00 | 32.76 |
| ATOM | 79 | CG2 | ILE | A | 9 | 109.208 | 28.781 | 10.012 | 1.00 | 29.90 |
| ATOM | 80 | CG1 | ILE | A | 9 | 107.803 | 27.454 | 11.633 | 1.00 | 32.76 |
| ATOM | 81 | CD1 | ILE | A | 9 | 108.945 | 27.087 | 12.554 | 1.00 | 36.13 |
| ATOM | 82 | C | ILE | A | 9 | 107.309 | 27.889 | 7.832 | 1.00 | 34.24 |
| ATOM | 83 | O | ILE | A | 9 | 107.650 | 26.845 | 7.265 | 1.00 | 37.01 |
| ATOM | 84 | N | SER | A | 10 | 107.179 | 29.045 | 7.195 | 1.00 | 34.50 |
| ATOM | 86 | CA | SER | A | 10 | 107.473 | 29.173 | 5.774 | 1.00 | 33.98 |
| ATOM | 87 | CB | SER | A | 10 | 106.536 | 30.200 | 5.146 | 1.00 | 33.86 |
| ATOM | 88 | OG | SER | A | 10 | 106.778 | 31.488 | 5.679 | 1.00 | 36.26 |
| ATOM | 90 | C | SER | A | 10 | 108.922 | 29.563 | 5.500 | 1.00 | 32.91 |
| ATOM | 91 | O | SER | A | 10 | 109.674 | 29.886 | 6.416 | 1.00 | 33.57 |
| ATOM | 92 | N | GLN | A | 11 | 109.300 | 29.550 | 4.225 | 1.00 | 34.16 |
| ATOM | 94 | CA | GLN | A | 11 | 110.653 | 29.912 | 3.811 | 1.00 | 35.54 |
| ATOM | 95 | CB | GLN | A | 11 | 110.792 | 29.845 | 2.283 | 1.00 | 35.39 |
| ATOM | 96 | CG | GLN | A | 11 | 112.212 | 30.070 | 1.746 | 1.00 | 36.23 |
| ATOM | 97 | CD | GLN | A | 11 | 113.164 | 28.920 | 2.040 | 1.00 | 35.94 |
| ATOM | 98 | OE1 | GLN | A | 11 | 112.880 | 27.774 | 1.716 | 1.00 | 38.97 |
| ATOM | 99 | NE2 | GLN | A | 11 | 114.310 | 29.230 | 2.624 | 1.00 | 35.28 |
| ATOM | 102 | C | GLN | A | 11 | 110.991 | 31.312 | 4.316 | 1.00 | 36.60 |
| ATOM | 103 | O | GLN | A | 11 | 112.116 | 31.559 | 4.740 | 1.00 | 41.10 |
| ATOM | 104 | N | LYS | A | 12 | 110.013 | 32.216 | 4.305 | 1.00 | 35.51 |
| ATOM | 106 | CA | LYS | A | 12 | 110.245 | 33.573 | 4.788 | 1.00 | 31.96 |
| ATOM | 107 | CB | LYS | A | 12 | 109.005 | 34.445 | 4.612 | 1.00 | 32.24 |
| ATOM | 108 | CG | LYS | A | 12 | 109.226 | 35.876 | 5.065 | 1.00 | 32.71 |
| ATOM | 109 | CD | LYS | A | 12 | 107.953 | 36.685 | 5.063 | 1.00 | 31.87 |
| ATOM | 110 | CE | LYS | A | 12 | 108.227 | 38.085 | 5.573 | 1.00 | 34.67 |
| ATOM | 111 | NZ | LYS | A | 12 | 106.998 | 38.914 | 5.627 | 1.00 | 37.88 |
| ATOM | 115 | C | LYS | A | 12 | 110.612 | 33.498 | 6.259 | 1.00 | 30.93 |
| ATOM | 116 | O | LYS | A | 12 | 111.482 | 34.227 | 6.728 | 1.00 | 32.37 |
| ATOM | 117 | N | GLY | A | 13 | 109.961 | 32.586 | 6.973 | 1.00 | 30.47 |
| ATOM | 119 | CA | GLY | A | 13 | 110.231 | 32.407 | 8.385 | 1.00 | 27.64 |
| ATOM | 120 | C | GLY | A | 13 | 111.593 | 31.791 | 8.610 | 1.00 | 29.49 |
| ATOM | 121 | O | GLY | A | 13 | 112.312 | 32.184 | 9.531 | 1.00 | 30.35 |
| ATOM | 122 | N | LEU | A | 14 | 111.952 | 30.817 | 7.779 | 1.00 | 28.87 |
| ATOM | 124 | CA | LEU | A | 14 | 113.248 | 30.170 | 7.912 | 1.00 | 28.53 |
| ATOM | 125 | CB | LEU | A | 14 | 113.361 | 28.941 | 7.011 | 1.00 | 25.60 |
| ATOM | 126 | CG | LEU | A | 14 | 112.564 | 27.698 | 7.419 | 1.00 | 25.99 |
| ATOM | 127 | CD1 | LEU | A | 14 | 112.959 | 26.547 | 6.531 | 1.00 | 28.34 |
| ATOM | 128 | CD2 | LEU | A | 14 | 112.829 | 27.330 | 8.855 | 1.00 | 23.90 |
| ATOM | 129 | C | LEU | A | 14 | 114.359 | 31.155 | 7.613 | 1.00 | 28.63 |
| ATOM | 130 | O | LEU | A | 14 | 115.346 | 31.207 | 8.335 | 1.00 | 31.86 |
| ATOM | 131 | N | ASP | A | 15 | 114.176 | 31.972 | 6.582 | 1.00 | 31.30 |
| ATOM | 133 | CA | ASP | A | 15 | 115.176 | 32.975 | 6.215 | 1.00 | 33.98 |
| ATOM | 134 | CB | ASP | A | 15 | 114.695 | 33.832 | 5.033 | 1.00 | 36.03 |
| ATOM | 135 | CG | ASP | A | 15 | 114.818 | 33.127 | 3.681 | 1.00 | 36.52 |
| ATOM | 136 | OD1 | ASP | A | 15 | 115.662 | 32.209 | 3.515 | 1.00 | 37.01 |
| ATOM | 137 | OD2 | ASP | A | 15 | 114.071 | 33.530 | 2.765 | 1.00 | 36.25 |
| ATOM | 138 | C | ASP | A | 15 | 115.473 | 33.882 | 7.413 | 1.00 | 35.08 |
| ATOM | 139 | O | ASP | A | 15 | 116.626 | 34.246 | 7.654 | 1.00 | 37.07 |
| ATOM | 140 | N | TYR | A | 16 | 114.433 | 34.244 | 8.162 | 1.00 | 34.55 |
| ATOM | 142 | CA | TYR | A | 16 | 114.598 | 35.098 | 9.337 | 1.00 | 34.48 |
| ATOM | 143 | CB | TYR | A | 16 | 113.239 | 35.554 | 9.865 | 1.00 | 34.89 |
| ATOM | 144 | CG | TYR | A | 16 | 113.320 | 36.661 | 10.891 | 1.00 | 35.28 |
| ATOM | 145 | CD1 | TYR | A | 16 | 113.859 | 37.905 | 10.562 | 1.00 | 36.36 |
| ATOM | 146 | CE1 | TYR | A | 16 | 113.921 | 38.936 | 11.496 | 1.00 | 34.26 |
| ATOM | 147 | CD2 | TYR | A | 16 | 112.846 | 36.474 | 12.182 | 1.00 | 33.51 |
| ATOM | 148 | CE2 | TYR | A | 16 | 112.903 | 37.498 | 13.118 | 1.00 | 34.97 |
| ATOM | 149 | CZ | TYR | A | 16 | 113.440 | 38.724 | 12.767 | 1.00 | 34.08 |
| ATOM | 150 | OH | TYR | A | 16 | 113.491 | 39.733 | 13.697 | 1.00 | 37.78 |
| ATOM | 152 | C | TYR | A | 16 | 115.348 | 34.340 | 10.427 | 1.00 | 35.46 |
| ATOM | 153 | O | TYR | A | 16 | 116.240 | 34.883 | 11.074 | 1.00 | 36.90 |
| ATOM | 154 | N | ALA | A | 17 | 114.982 | 33.081 | 10.625 | 1.00 | 36.20 |
| ATOM | 156 | CA | ALA | A | 17 | 115.632 | 32.250 | 11.617 | 1.00 | 35.77 |
| ATOM | 157 | CB | ALA | A | 17 | 115.025 | 30.870 | 11.615 | 1.00 | 33.78 |
| ATOM | 158 | C | ALA | A | 17 | 117.118 | 32.180 | 11.293 | 1.00 | 37.85 |
| ATOM | 159 | O | ALA | A | 17 | 117.957 | 32.330 | 12.178 | 1.00 | 40.31 |
| ATOM | 160 | N | SER | A | 18 | 117.447 | 32.008 | 10.017 | 1.00 | 39.36 |
| ATOM | 162 | CA | SER | A | 18 | 118.846 | 31.927 | 9.612 | 1.00 | 42.40 |
| ATOM | 163 | CB | SER | A | 18 | 118.993 | 31.399 | 8.175 | 1.00 | 43.78 |
| ATOM | 164 | OG | SER | A | 18 | 118.220 | 32.127 | 7.236 | 1.00 | 48.26 |
| ATOM | 166 | C | SER | A | 18 | 119.605 | 33.238 | 9.786 | 1.00 | 42.53 |
| ATOM | 167 | O | SER | A | 18 | 120.768 | 33.228 | 10.178 | 1.00 | 44.52 |
| ATOM | 168 | N | GLN | A | 19 | 118.959 | 34.367 | 9.509 | 1.00 | 42.42 |
| ATOM | 170 | CA | GLN | A | 19 | 119.630 | 35.652 | 9.665 | 1.00 | 42.67 |
| ATOM | 171 | CB | GLN | A | 19 | 118.805 | 36.806 | 9.089 | 1.00 | 47.13 |
| ATOM | 172 | CG | GLN | A | 19 | 118.810 | 36.864 | 7.563 | 1.00 | 57.52 |
| ATOM | 173 | CD | GLN | A | 19 | 118.457 | 38.238 | 7.012 | 1.00 | 62.74 |
| ATOM | 174 | OE1 | GLN | A | 19 | 117.488 | 38.866 | 7.440 | 1.00 | 65.88 |
| ATOM | 175 | NE2 | GLN | A | 19 | 119.248 | 38.710 | 6.051 | 1.00 | 64.58 |
| ATOM | 178 | C | GLN | A | 19 | 119.943 | 35.906 | 11.126 | 1.00 | 40.97 |
| ATOM | 179 | O | GLN | A | 19 | 121.030 | 36.378 | 11.456 | 1.00 | 41.85 |
| ATOM | 180 | N | GLN | A | 20 | 119.008 | 35.565 | 12.005 | 1.00 | 39.51 |
| ATOM | 182 | CA | GLN | A | 20 | 119.226 | 35.759 | 13.430 | 1.00 | 37.61 |
| ATOM | 183 | CB | GLN | A | 20 | 117.944 | 35.507 | 14.228 | 1.00 | 38.26 |
| ATOM | 184 | CG | GLN | A | 20 | 116.764 | 36.405 | 13.874 | 1.00 | 38.33 |
| ATOM | 185 | CD | GLN | A | 20 | 117.057 | 37.895 | 14.002 | 1.00 | 41.17 |
| ATOM | 186 | OE1 | GLN | A | 20 | 116.912 | 38.642 | 13.040 | 1.00 | 43.42 |
| ATOM | 187 | NE2 | GLN | A | 20 | 117.441 | 38.335 | 15.192 | 1.00 | 42.94 |
| ATOM | 190 | C | GLN | A | 20 | 120.325 | 34.805 | 13.878 | 1.00 | 36.56 |
| ATOM | 191 | O | GLN | A | 20 | 121.208 | 35.176 | 14.654 | 1.00 | 38.07 |
| ATOM | 192 | N | GLY | A | 21 | 120.285 | 33.587 | 13.348 | 1.00 | 36.41 |
| ATOM | 194 | CA | GLY | A | 21 | 121.276 | 32.585 | 13.688 | 1.00 | 33.84 |
| ATOM | 195 | C | GLY | A | 21 | 122.665 | 33.023 | 13.289 | 1.00 | 34.61 |
| ATOM | 196 | O | GLY | A | 21 | 123.520 | 33.199 | 14.140 | 1.00 | 36.02 |
| ATOM | 197 | N | THR | A | 22 | 122.879 | 33.221 | 11.994 | 1.00 | 35.98 |
| ATOM | 199 | CA | THR | A | 22 | 124.174 | 33.645 | 11.462 | 1.00 | 39.75 |
| ATOM | 200 | CB | THR | A | 22 | 24.036 | 34.109 | 9.983 | 1.00 | 41.34 |
| ATOM | 201 | OG1 | THR | A | 22 | 123.400 | 33.082 | 9.212 | 1.00 | 40.97 |
| ATOM | 203 | CG2 | THR | A | 22 | 125.397 | 34.408 | 9.377 | 1.00 | 40.54 |
| ATOM | 204 | C | THR | A | 22 | 124.780 | 34.785 | 12.285 | 1.00 | 40.46 |
| ATOM | 205 | O | THR | A | 22 | 125.954 | 34.741 | 12.667 | 1.00 | 41.11 |
| ATOM | 206 | N | ALA | A | 23 | 123.959 | 35.787 | 12.578 | 1.00 | 41.75 |
| ATOM | 208 | CA | ALA | A | 23 | 124.387 | 36.944 | 13.351 | 1.00 | 41.89 |
| ATOM | 209 | CB | ALA | A | 23 | 123.224 | 37.895 | 13.567 | 1.00 | 40.51 |
| ATOM | 210 | C | ALA | A | 23 | 124.919 | 36.461 | 14.678 | 1.00 | 43.71 |
| ATOM | 211 | O | ALA | A | 23 | 125.992 | 36.872 | 15.114 | 1.00 | 47.24 |
| ATOM | 212 | N | ALA | A | 24 | 124.175 | 35.554 | 15.298 | 1.00 | 43.71 |
| ATOM | 214 | CA | ALA | A | 24 | 124.570 | 34.991 | 16.574 | 1.00 | 43.02 |
| ATOM | 215 | CB | ALA | A | 24 | 123.413 | 34.238 | 17.185 | 1.00 | 43.37 |
| ATOM | 216 | C | ALA | A | 24 | 125.774 | 34.069 | 16.396 | 1.00 | 42.93 |
| ATOM | 217 | O | ALA | A | 24 | 126.549 | 33.868 | 17.328 | 1.00 | 45.41 |
| ATOM | 218 | N | LEU | A | 25 | 125.944 | 33.532 | 15.193 | 1.00 | 41.61 |
| ATOM | 220 | CA | LEU | A | 25 | 127.056 | 32.638 | 14.910 | 1.00 | 40.32 |
| ATOM | 221 | CB | LEU | A | 25 | 126.746 | 31.755 | 13.699 | 1.00 | 36.89 |
| ATOM | 222 | CG | LEU | A | 25 | 127.662 | 30.554 | 13.483 | 1.00 | 34.95 |
| ATOM | 223 | CD1 | LEU | A | 25 | 127.636 | 29.653 | 14.703 | 1.00 | 34.64 |
| ATOM | 224 | CD2 | LEU | A | 25 | 127.218 | 29.797 | 12.262 | 1.00 | 35.16 |
| ATOM | 225 | C | LEU | A | 25 | 128.331 | 33.438 | 14.676 | 1.00 | 40.69 |
| ATOM | 226 | O | LEU | A | 25 | 129.423 | 32.984 | 15.014 | 1.00 | 42.61 |
| ATOM | 227 | N | GLN | A | 26 | 128.194 | 34.644 | 14.138 | 1.00 | 41.51 |
| ATOM | 229 | CA | GLN | A | 26 | 129.352 | 35.494 | 13.878 | 1.00 | 45.29 |
| ATOM | 230 | CB | GLN | A | 26 | 128.920 | 36.850 | 13.317 | 1.00 | 46.09 |
| ATOM | 231 | CG | GLN | A | 26 | 130.087 | 37.811 | 13.108 | 1.00 | 48.70 |
| ATOM | 232 | CD | GLN | A | 26 | 129.650 | 39.240 | 12.861 | 1.00 | 50.15 |
| ATOM | 233 | OE1 | GLN | A | 26 | 129.833 | 40.115 | 13.708 | 1.00 | 51.16 |
| ATOM | 234 | NE2 | GLN | A | 26 | 129.078 | 39.489 | 11.692 | 1.00 | 51.55 |
| ATOM | 237 | C | GLN | A | 26 | 130.175 | 35.728 | 15.145 | 1.00 | 46.79 |
| ATOM | 238 | O | GLN | A | 26 | 131.403 | 35.587 | 15.136 | 1.00 | 46.74 |
| ATOM | 239 | N | LYS | A | 27 | 129.488 | 36.081 | 16.230 | 1.00 | 48.40 |
| ATOM | 241 | CA | LYS | A | 27 | 130.140 | 36.355 | 17.509 | 1.00 | 50.23 |
| ATOM | 242 | CB | LYS | A | 27 | 129.119 | 36.696 | 18.595 | 1.00 | 52.58 |
| ATOM | 243 | CG | LYS | A | 27 | 128.157 | 37.818 | 18.249 | 1.00 | 55.93 |
| ATOM | 244 | CD | LYS | A | 27 | 127.495 | 38.391 | 19.503 | 1.00 | 59.98 |
| ATOM | 245 | CE | LYS | A | 27 | 126.959 | 37.306 | 20.437 | 1.00 | 63.01 |
| ATOM | 246 | NZ | LYS | A | 27 | 125.911 | 36.452 | 19.807 | 1.00 | 66.76 |
| ATOM | 250 | C | LYS | A | 27 | 130.983 | 35.186 | 17.977 | 1.00 | 50.39 |
| ATOM | 251 | O | LYS | A | 27 | 132.121 | 35.369 | 18.398 | 1.00 | 52.00 |
| ATOM | 252 | N | GLU | A | 28 | 130.421 | 33.984 | 17.920 | 1.00 | 50.09 |
| ATOM | 254 | CA | GLU | A | 28 | 131.157 | 32.803 | 18.341 | 1.00 | 48.77 |
| ATOM | 255 | CB | GLU | A | 28 | 130.250 | 31.571 | 18.390 | 1.00 | 48.51 |
| ATOM | 256 | CG | GLU | A | 28 | 129.514 | 31.395 | 19.713 | 1.00 | 50.95 |
| ATOM | 257 | CD | GLU | A | 28 | 130.436 | 30.999 | 20.857 | 1.00 | 52.82 |
| ATOM | 258 | OE1 | GLU | A | 28 | 130.687 | 29.789 | 21.030 | 1.00 | 55.51 |
| ATOM | 259 | OE2 | GLU | A | 28 | 130.903 | 31.891 | 21.594 | 1.00 | 54.60 |
| ATOM | 260 | C | GLU | A | 28 | 132.343 | 32.564 | 17.421 | 1.00 | 46.96 |
| ATOM | 261 | O | GLU | A | 28 | 133.407 | 32.153 | 17.871 | 1.00 | 49.87 |
| ATOM | 262 | N | LEU | A | 29 | 132.177 | 32.868 | 16.140 | 1.00 | 43.03 |
| ATOM | 264 | CA | LEU | A | 29 | 133.257 | 32.674 | 15.189 | 1.00 | 39.75 |
| ATOM | 265 | CB | LEU | A | 29 | 132.740 | 32.817 | 13.763 | 1.00 | 36.79 |
| ATOM | 266 | CG | LEU | A | 29 | 131.832 | 31.658 | 13.360 | 1.00 | 33.73 |
| ATOM | 267 | CD1 | LEU | A | 29 | 131.557 | 31.708 | 11.870 | 1.00 | 34.40 |
| ATOM | 268 | CD2 | LEU | A | 29 | 132.499 | 30.347 | 13.720 | 1.00 | 31.01 |
| ATOM | 269 | C | LEU | A | 29 | 134.431 | 33.610 | 15.444 | 1.00 | 38.40 |
| ATOM | 270 | O | LEU | A | 29 | 135.584 | 33.204 | 15.348 | 1.00 | 38.06 |
| ATOM | 271 | N | LYS | A | 30 | 134.131 | 34.843 | 15.828 | 1.00 | 38.44 |
| ATOM | 273 | CA | LYS | A | 30 | 135.155 | 35.845 | 16.111 | 1.00 | 40.67 |
| ATOM | 274 | CB | LYS | A | 30 | 134.495 | 37.225 | 16.210 | 1.00 | 41.05 |
| ATOM | 275 | CG | LYS | A | 30 | 135.448 | 38.409 | 16.270 | 1.00 | 43.32 |
| ATOM | 276 | CD | LYS | A | 30 | 134.690 | 39.733 | 16.198 | 1.00 | 44.53 |
| ATOM | 277 | CE | LYS | A | 30 | 135.633 | 40.926 | 16.343 | 1.00 | 46.81 |
| ATOM | 278 | NZ | LYS | A | 30 | 134.912 | 42.238 | 16.331 | 1.00 | 50.27 |
| ATOM | 282 | C | LYS | A | 30 | 135.955 | 35.539 | 17.388 | 1.00 | 42.29 |
| ATOM | 283 | O | LYS | A | 30 | 136.979 | 36.166 | 17.654 | 1.00 | 42.94 |
| ATOM | 284 | N | ARG | A | 31 | 135.495 | 34.565 | 18.168 | 1.00 | 44.02 |
| ATOM | 286 | CA | ARG | A | 31 | 136.162 | 34.190 | 19.411 | 1.00 | 44.45 |
| ATOM | 287 | CB | ARG | A | 31 | 135.139 | 34.005 | 20.534 | 1.00 | 46.86 |
| ATOM | 288 | CG | ARG | A | 31 | 134.504 | 35.305 | 20.983 | 1.00 | 52.93 |
| ATOM | 289 | CD | ARG | A | 31 | 133.479 | 35.095 | 22.078 | 1.00 | 60.04 |
| ATOM | 290 | NE | ARG | A | 31 | 132.947 | 36.372 | 22.554 | 1.00 | 67.90 |
| ATOM | 292 | CZ | ARG | A | 31 | 131.839 | 36.505 | 23.279 | 1.00 | 71.80 |
| ATOM | 293 | NH1 | ARG | A | 31 | 131.128 | 35.437 | 23.621 | 1.00 | 74.30 |
| ATOM | 296 | NH2 | ARG | A | 31 | 131.444 | 37.710 | 23.672 | 1.00 | 73.67 |
| ATOM | 299 | C | ARG | A | 31 | 137.008 | 32.937 | 19.276 | 1.00 | 44.41 |
| ATOM | 300 | O | ARG | A | 31 | 137.339 | 32.293 | 20.273 | 1.00 | 46.21 |
| ATOM | 301 | N | ILE | A | 32 | 137.343 | 32.581 | 18.041 | 1.00 | 44.30 |
| ATOM | 303 | CA | ILE | A | 32 | 138.162 | 31.404 | 17.783 | 1.00 | 42.96 |
| ATOM | 304 | CB | ILE | A | 32 | 138.054 | 30.953 | 16.308 | 1.00 | 39.50 |
| ATOM | 305 | CG2 | ILE | A | 32 | 139.146 | 29.950 | 15.973 | 1.00 | 36.12 |
| ATOM | 306 | CG1 | ILE | A | 32 | 136.674 | 30.351 | 16.052 | 1.00 | 37.18 |
| ATOM | 307 | CD1 | ILE | A | 32 | 136.431 | 29.975 | 14.624 | 1.00 | 37.20 |
| ATOM | 308 | C | ILE | A | 32 | 139.613 | 31.707 | 18.103 | 1.00 | 44.44 |
| ATOM | 309 | O | ILE | A | 32 | 140.109 | 32.794 | 17.798 | 1.00 | 45.60 |
| ATOM | 310 | N | LYS | A | 33 | 140.288 | 30.747 | 18.725 | 1.00 | 45.45 |
| ATOM | 312 | CA | LYS | A | 33 | 141.692 | 30.917 | 19.060 | 1.00 | 46.20 |
| ATOM | 313 | CB | LYS | A | 33 | 141.958 | 30.569 | 20.522 | 1.00 | 49.33 |
| ATOM | 314 | CG | LYS | A | 33 | 141.336 | 31.546 | 21.492 | 1.00 | 52.41 |
| ATOM | 315 | CD | LYS | A | 33 | 141.910 | 31.387 | 22.885 | 1.00 | 56.15 |
| ATOM | 316 | CE | LYS | A | 33 | 143.374 | 31.789 | 22.918 | 1.00 | 59.63 |
| ATOM | 317 | NZ | LYS | A | 33 | 143.891 | 31.817 | 24.313 | 1.00 | 63.31 |
| ATOM | 321 | C | LYS | A | 33 | 142.619 | 30.115 | 18.153 | 1.00 | 44.41 |
| ATOM | 322 | O | LYS | A | 33 | 142.563 | 28.884 | 18.102 | 1.00 | 42.99 |
| ATOM | 323 | N | ILE | A | 34 | 143.442 | 30.836 | 17.408 | 1.00 | 43.49 |
| ATOM | 325 | CA | ILE | A | 34 | 144.411 | 30.231 | 16.512 | 1.00 | 43.47 |
| ATOM | 326 | CB | ILE | A | 34 | 144.452 | 30.958 | 15.159 | 1.00 | 41.93 |
| ATOM | 327 | CG2 | ILE | A | 34 | 143.322 | 30.464 | 14.280 | 1.00 | 41.39 |
| ATOM | 328 | CG1 | ILE | A | 34 | 144.347 | 32.470 | 15.363 | 1.00 | 42.97 |
| ATOM | 329 | CD1 | ILE | A | 34 | 143.967 | 33.234 | 14.108 | 1.00 | 44.59 |
| ATOM | 330 | C | ILE | A | 34 | 145.761 | 30.288 | 17.216 | 1.00 | 44.81 |
| ATOM | 331 | O | ILE | A | 34 | 146.142 | 31.319 | 17.778 | 1.00 | 46.96 |
| ATOM | 332 | N | PRO | A | 35 | 146.464 | 29.151 | 17.267 | 1.00 | 43.85 |
| ATOM | 333 | CD | PRO | A | 35 | 146.008 | 27.862 | 16.728 | 1.00 | 44.63 |
| ATOM | 334 | CA | PRO | A | 35 | 147.773 | 29.003 | 17.904 | 1.00 | 42.65 |
| ATOM | 335 | CB | PRO | A | 35 | 148.109 | 27.534 | 17.650 | 1.00 | 42.05 |
| ATOM | 336 | CG | PRO | A | 35 | 146.778 | 26.887 | 17.578 | 1.00 | 44.72 |
| ATOM | 337 | C | PRO | A | 35 | 148.860 | 29.899 | 17.334 | 1.00 | 43.14 |
| ATOM | 338 | O | PRO | A | 35 | 148.649 | 30.640 | 16.376 | 1.00 | 44.26 |
| ATOM | 339 | N | ASP | A | 36 | 150.026 | 29.827 | 17.959 | 1.00 | 42.32 |
| ATOM | 341 | CA | ASP | A | 36 | 151.181 | 30.584 | 17.535 | 1.00 | 41.85 |
| ATOM | 342 | CB | ASP | A | 36 | 151.933 | 31.125 | 18.745 | 1.00 | 43.04 |
| ATOM | 343 | CG | ASP | A | 36 | 151.155 | 32.196 | 19.485 | 1.00 | 45.33 |
| ATOM | 344 | OD1 | ASP | A | 36 | 150.120 | 32.666 | 18.959 | 1.00 | 49.40 |
| ATOM | 345 | OD2 | ASP | A | 36 | 151.591 | 32.584 | 20.588 | 1.00 | 46.99 |
| ATOM | 346 | C | ASP | A | 36 | 152.051 | 29.612 | 16.776 | 1.00 | 42.29 |
| ATOM | 347 | O | ASP | A | 36 | 152.175 | 28.450 | 17.159 | 1.00 | 43.32 |
| ATOM | 348 | N | TYR | A | 37 | 152.637 | 30.077 | 15.687 | 1.00 | 42.04 |
| ATOM | 350 | CA | TYR | A | 37 | 153.476 | 29.228 | 14.877 | 1.00 | 41.59 |
| ATOM | 351 | CB | TYR | A | 37 | 152.979 | 29.243 | 13.436 | 1.00 | 42.04 |
| ATOM | 352 | CG | TYR | A | 37 | 151.564 | 28.743 | 13.327 | 1.00 | 43.15 |
| ATOM | 353 | CD1 | TYR | A | 37 | 151.295 | 27.378 | 13.256 | 1.00 | 42.32 |
| ATOM | 354 | CE1 | TYR | A | 37 | 149.991 | 26.904 | 13.239 | 1.00 | 43.09 |
| ATOM | 355 | CD2 | TYR | A | 37 | 150.487 | 29.626 | 13.370 | 1.00 | 44.59 |
| ATOM | 356 | CE2 | TYR | A | 37 | 149.175 | 29.161 | 13.354 | 1.00 | 43.64 |
| ATOM | 357 | CZ | TYR | A | 37 | 148.938 | 27.799 | 13.291 | 1.00 | 42.12 |
| ATOM | 358 | OH | TYR | A | 37 | 147.651 | 27.327 | 13.307 | 1.00 | 43.31 |
| ATOM | 360 | C | TYR | A | 37 | 154.923 | 29.649 | 14.957 | 1.00 | 42.22 |
| ATOM | 361 | O | TYR | A | 37 | 155.311 | 30.692 | 14.443 | 1.00 | 43.15 |
| ATOM | 362 | N | SER | A | 38 | 155.714 | 28.833 | 15.634 | 1.00 | 44.23 |
| ATOM | 364 | CA | SER | A | 38 | 157.130 | 29.088 | 15.789 | 1.00 | 47.19 |
| ATOM | 365 | CB | SER | A | 38 | 157.466 | 29.330 | 17.257 | 1.00 | 47.44 |
| ATOM | 366 | OG | SER | A | 38 | 156.651 | 30.356 | 17.799 | 1.00 | 54.54 |
| ATOM | 368 | C | SER | A | 38 | 157.887 | 27.878 | 15.285 | 1.00 | 48.36 |
| ATOM | 369 | O | SER | A | 38 | 157.664 | 26.761 | 15.746 | 1.00 | 49.57 |
| ATOM | 370 | N | ASP | A | 39 | 158.773 | 28.103 | 14.327 | 1.00 | 50.18 |
| ATOM | 372 | CA | ASP | A | 39 | 159.569 | 27.030 | 13.763 | 1.00 | 52.06 |
| ATOM | 373 | CB | ASP | A | 39 | 158.822 | 26.402 | 12.579 | 1.00 | 53.01 |
| ATOM | 374 | CG | ASP | A | 39 | 159.375 | 25.043 | 12.179 | 1.00 | 54.91 |
| ATOM | 375 | OD1 | ASP | A | 39 | 159.569 | 24.178 | 13.063 | 1.00 | 54.67 |
| ATOM | 376 | OD2 | ASP | A | 39 | 159.600 | 24.837 | 10.969 | 1.00 | 56.72 |
| ATOM | 377 | C | ASP | A | 39 | 160.912 | 27.631 | 13.334 | 1.00 | 53.93 |
| ATOM | 378 | O | ASP | A | 39 | 161.100 | 28.853 | 13.384 | 1.00 | 54.67 |
| ATOM | 379 | N | SER | A | 40 | 161.868 | 26.771 | 13.003 | 1.00 | 54.19 |
| ATOM | 381 | CA | SER | A | 40 | 163.187 | 27.208 | 12.577 | 1.00 | 52.94 |
| ATOM | 382 | CB | SER | A | 40 | 164.255 | 26.307 | 13.202 | 1.00 | 52.97 |
| ATOM | 383 | OG | SER | A | 40 | 163.996 | 24.934 | 12.947 | 1.00 | 53.27 |
| ATOM | 385 | C | SER | A | 40 | 163.283 | 27.163 | 11.057 | 1.00 | 54.32 |
| ATOM | 386 | O | SER | A | 40 | 162.394 | 26.623 | 10.386 | 1.00 | 54.83 |
| ATOM | 387 | N | PHE | A | 41 | 164.358 | 27.730 | 10.516 | 1.00 | 55.12 |
| ATOM | 389 | CA | PHE | A | 41 | 164.580 | 27.742 | 9.074 | 1.00 | 55.04 |
| ATOM | 390 | CB | PHE | A | 41 | 163.908 | 28.967 | 8.424 | 1.00 | 50.91 |
| ATOM | 391 | CG | PHE | A | 41 | 164.564 | 30.285 | 8.756 | 1.00 | 46.23 |
| ATOM | 392 | CD1 | PHE | A | 41 | 165.720 | 30.695 | 8.092 | 1.00 | 44.94 |
| ATOM | 393 | CD2 | PHE | A | 41 | 164.019 | 31.124 | 9.719 | 1.00 | 44.37 |
| ATOM | 394 | CE1 | PHE | A | 41 | 166.321 | 31.918 | 8.386 | 1.00 | 43.85 |
| ATOM | 395 | CE2 | PHE | A | 41 | 164.614 | 32.350 | 10.018 | 1.00 | 43.51 |
| ATOM | 396 | CZ | PHE | A | 41 | 165.765 | 32.746 | 9.351 | 1.00 | 42.57 |
| ATOM | 397 | C | PHE | A | 41 | 166.071 | 27.724 | 8.756 | 1.00 | 56.75 |
| ATOM | 398 | O | PHE | A | 41 | 166.904 | 27.935 | 9.637 | 1.00 | 58.20 |
| ATOM | 399 | N | LYS | A | 42 | 166.391 | 27.455 | 7.496 | 1.00 | 59.71 |
| ATOM | 401 | CA | LYS | A | 42 | 167.768 | 27.427 | 7.010 | 1.00 | 63.27 |
| ATOM | 402 | CB | LYS | A | 42 | 168.474 | 26.129 | 7.425 | 1.00 | 64.98 |
| ATOM | 403 | CG | LYS | A | 42 | 169.984 | 26.140 | 7.215 | 1.00 | 68.46 |
| ATOM | 404 | CD | LYS | A | 42 | 170.706 | 25.591 | 8.449 | 1.00 | 70.94 |
| ATOM | 405 | CE | LYS | A | 42 | 172.227 | 25.630 | 8.298 | 1.00 | 71.66 |
| ATOM | 406 | NZ | LYS | A | 42 | 172.928 | 25.300 | 9.582 | 1.00 | 71.11 |
| ATOM | 410 | C | LYS | A | 42 | 167.706 | 27.552 | 5.487 | 1.00 | 64.24 |
| ATOM | 411 | O | LYS | A | 42 | 167.585 | 26.555 | 4.773 | 1.00 | 65.07 |
| ATOM | 412 | N | ILE | A | 43 | 167.725 | 28.792 | 5.003 | 1.00 | 64.98 |
| ATOM | 414 | CA | ILE | A | 43 | 167.652 | 29.070 | 3.570 | 1.00 | 64.79 |
| ATOM | 415 | CB | ILE | A | 43 | 166.721 | 30.282 | 3.282 | 1.00 | 65.08 |
| ATOM | 416 | CG2 | ILE | A | 43 | 166.731 | 30.617 | 1.796 | 1.00 | 66.08 |
| ATOM | 417 | CG1 | ILE | A | 43 | 165.293 | 29.965 | 3.745 | 1.00 | 64.11 |
| ATOM | 418 | CD1 | ILE | A | 43 | 164.284 | 31.071 | 3.478 | 1.00 | 65.50 |
| ATOM | 419 | C | ILE | A | 43 | 169.039 | 29.291 | 2.956 | 1.00 | 64.78 |
| ATOM | 420 | O | ILE | A | 43 | 169.840 | 30.079 | 3.465 | 1.00 | 65.23 |
| ATOM | 421 | N | LYS | A | 44 | 169.273 | 28.631 | 1.825 | 1.00 | 64.51 |
| ATOM | 423 | CA | LYS | A | 44 | 170.539 | 28.672 | 1.093 | 1.00 | 63.65 |
| ATOM | 424 | CB | LYS | A | 44 | 170.328 | 28.232 | −0.360 | 1.00 | 63.66 |
| ATOM | 425 | CG | LYS | A | 44 | 170.066 | 26.742 | −0.553 | 1.00 | 63.81 |
| ATOM | 426 | CD | LYS | A | 44 | 170.064 | 26.396 | −2.035 | 1.00 | 65.27 |
| ATOM | 427 | CE | LYS | A | 44 | 169.910 | 24.905 | −2.289 | 1.00 | 65.86 |
| ATOM | 428 | NZ | LYS | A | 44 | 169.953 | 24.599 | −3.751 | 1.00 | 66.87 |
| ATOM | 432 | C | LYS | A | 44 | 171.382 | 29.947 | 1.115 | 1.00 | 63.16 |
| ATOM | 433 | O | LYS | A | 44 | 172.600 | 29.876 | 1.274 | 1.00 | 65.20 |
| ATOM | 434 | N | HIS | A | 45 | 170.769 | 31.107 | 0.929 | 1.00 | 61.08 |
| ATOM | 436 | CA | HIS | A | 45 | 171.550 | 32.339 | 0.920 | 1.00 | 61.08 |
| atom | 437 | CB | HIS | A | 45 | 171.454 | 33.017 | −0.447 | 1.00 | 64.62 |
| ATOM | 438 | CG | HIS | A | 45 | 172.039 | 32.214 | −1.565 | 1.00 | 68.00 |
| ATOM | 439 | CD2 | HIS | A | 45 | 171.695 | 32.126 | −2.871 | 1.00 | 68.82 |
| ATOM | 440 | ND1 | HIS | A | 45 | 173.124 | 31.380 | −1.397 | 1.00 | 70.18 |
| ATOM | 442 | CE1 | HIS | A | 45 | 173.425 | 30.815 | −2.552 | 1.00 | 71.24 |
| ATOM | 443 | NE2 | HIS | A | 45 | 172.572 | 31.250 | −3.462 | 1.00 | 70.61 |
| ATOM | 445 | C | HIS | A | 45 | 171.167 | 33.337 | 1.994 | 1.00 | 59.87 |
| ATOM | 446 | O | HIS | A | 45 | 171.609 | 34.486 | 1.960 | 1.00 | 60.80 |
| ATOM | 447 | N | LEU | A | 46 | 170.345 | 32.900 | 2.940 | 1.00 | 57.18 |
| ATOM | 449 | CA | LEU | A | 46 | 169.881 | 33.767 | 4.012 | 1.00 | 53.69 |
| ATOM | 450 | CB | LEU | A | 46 | 168.362 | 33.699 | 4.103 | 1.00 | 53.23 |
| ATOM | 451 | CG | LEU | A | 46 | 167.715 | 34.609 | 5.135 | 1.00 | 51.73 |
| ATOM | 452 | CG1 | LEU | A | 46 | 167.712 | 36.039 | 4.628 | 1.00 | 52.65 |
| ATOM | 453 | CD2 | LEU | A | 46 | 166.310 | 34.126 | 5.390 | 1.00 | 51.01 |
| ATOM | 454 | C | LEU | A | 46 | 170.490 | 33.336 | 5.330 | 1.00 | 52.64 |
| ATOM | 455 | O | LEU | A | 46 | 170.908 | 34.168 | 6.136 | 1.00 | 52.10 |
| ATOM | 456 | N | GLY | A | 47 | 170.513 | 32.026 | 5.550 | 1.00 | 51.88 |
| ATOM | 458 | CA | GLY | A | 47 | 171.081 | 31.486 | 6.768 | 1.00 | 51.77 |
| ATOM | 459 | C | GLY | A | 47 | 170.107 | 30.643 | 7.564 | 1.00 | 51.44 |
| ATOM | 460 | O | GLY | A | 47 | 169.059 | 30.235 | 7.065 | 1.00 | 50.21 |
| ATOM | 461 | N | LYS | A | 48 | 170.484 | 30.357 | 8.802 | 1.00 | 52.15 |
| ATOM | 463 | CA | LYS | A | 48 | 169.664 | 29.572 | 9.711 | 1.00 | 52.12 |
| ATOM | 464 | CB | LYS | A | 48 | 170.497 | 28.479 | 10.407 | 1.00 | 56.73 |
| ATOM | 465 | CG | LYS | A | 48 | 171.878 | 28.907 | 10.938 | 1.00 | 62.50 |
| ATOM | 466 | CD | LYS | A | 48 | 172.953 | 28.876 | 9.843 | 1.00 | 65.46 |
| ATOM | 467 | CE | LYS | A | 48 | 174.332 | 29.252 | 10.380 | 1.00 | 66.23 |
| ATOM | 468 | NZ | LYS | A | 48 | 175.380 | 29.178 | 9.318 | 1.00 | 65.64 |
| ATOM | 472 | C | LYS | A | 48 | 169.041 | 30.510 | 10.738 | 1.00 | 49.63 |
| ATOM | 473 | O | LYS | A | 48 | 169.632 | 31.533 | 11.101 | 1.00 | 48.85 |
| ATOM | 474 | N | GLY | A | 49 | 167.846 | 30.174 | 11.200 | 1.00 | 46.79 |
| ATOM | 476 | CA | GLY | A | 49 | 167.197 | 31.026 | 12.169 | 1.00 | 47.22 |
| ATOM | 477 | C | GLY | A | 49 | 165.918 | 30.449 | 12.728 | 1.00 | 47.47 |
| ATOM | 478 | O | GLY | A | 49 | 165.594 | 29.285 | 12.506 | 1.00 | 46.51 |
| ATOM | 479 | N | HIS | A | 50 | 165.166 | 31.292 | 13.420 | 1.00 | 47.82 |
| ATOM | 481 | CA | HIS | A | 50 | 163.922 | 30.886 | 14.041 | 1.00 | 48.80 |
| ATOM | 482 | CB | HIS | A | 50 | 164.126 | 30.793 | 15.554 | 1.00 | 58.27 |
| ATOM | 483 | CG | HIS | A | 50 | 163.658 | 29.505 | 16.155 | 1.00 | 68.69 |
| ATOM | 484 | CD2 | HIS | A | 50 | 164.349 | 28.453 | 16.657 | 1.00 | 72.98 |
| ATOM | 485 | ND1 | HIS | A | 50 | 162.324 | 29.196 | 16.311 | 1.00 | 73.69 |
| ATOM | 487 | CE1 | HIS | A | 50 | 162.212 | 28.010 | 16.885 | 1.00 | 76.58 |
| ATOM | 488 | NE2 | HIS | A | 50 | 163.426 | 27.539 | 17.105 | 1.00 | 77.46 |
| ATOM | 490 | C | HIS | A | 50 | 162.905 | 31.965 | 13.742 | 1.00 | 45.89 |
| ATOM | 491 | O | HIS | A | 50 | 163.149 | 33.137 | 14.019 | 1.00 | 41.84 |
| ATOM | 492 | N | TYR | A | 51 | 161.785 | 31.585 | 13.143 | 1.00 | 46.60 |
| ATOM | 494 | CA | TYR | A | 51 | 160.746 | 32.555 | 12.824 | 1.00 | 48.52 |
| ATOM | 495 | CB | TYR | A | 51 | 160.308 | 32.420 | 11.354 | 1.00 | 50.04 |
| ATOM | 496 | CG | TYR | A | 51 | 159.360 | 31.280 | 11.098 | 1.00 | 51.79 |
| ATOM | 497 | CD1 | TYR | A | 51 | 157.987 | 31.449 | 11.266 | 1.00 | 53.21 |
| ATOM | 498 | CE1 | TYR | A | 51 | 157.111 | 30.396 | 11.103 | 1.00 | 56.71 |
| ATOM | 499 | CD2 | TYR | A | 51 | 159.833 | 30.021 | 10.745 | 1.00 | 52.56 |
| ATOM | 500 | CE2 | TYR | A | 51 | 158.959 | 28.955 | 10.573 | 1.00 | 56.44 |
| ATOM | 501 | CZ | TYR | A | 51 | 157.597 | 29.149 | 10.761 | 1.00 | 58.01 |
| ATOM | 502 | OH | TYR | A | 51 | 156.717 | 28.094 | 10.649 | 1.00 | 60.76 |
| ATOM | 504 | C | TYR | A | 51 | 159.566 | 32.330 | 13.763 | 1.00 | 47.31 |
| ATOM | 505 | O | TYR | A | 51 | 159.485 | 31.291 | 14.424 | 1.00 | 47.36 |
| ATOM | 506 | N | SER | A | 52 | 158.648 | 33.288 | 13.812 | 1.00 | 46.53 |
| ATOM | 508 | CA | SER | A | 52 | 157.480 | 33.163 | 14.669 | 1.00 | 43.69 |
| ATOM | 509 | CB | SER | A | 52 | 157.862 | 33.449 | 16.123 | 1.00 | 43.87 |
| ATOM | 510 | OG | SER | A | 52 | 156.759 | 33.233 | 16.986 | 1.00 | 42.88 |
| ATOM | 512 | C | SER | A | 52 | 156.344 | 34.083 | 14.253 | 1.00 | 41.60 |
| ATOM | 513 | O | SER | A | 52 | 156.558 | 35.261 | 13.981 | 1.00 | 42.86 |
| ATOM | 514 | N | PHE | A | 53 | 155.152 | 33.513 | 14.132 | 1.00 | 39.20 |
| ATOM | 516 | CA | PHE | A | 53 | 153.944 | 34.260 | 13.796 | 1.00 | 36.73 |
| ATOM | 517 | CB | PHE | A | 53 | 153.310 | 33.737 | 12.507 | 1.00 | 33.91 |
| ATOM | 518 | CG | PHE | A | 53 | 154.107 | 34.054 | 11.271 | 1.00 | 32.26 |
| ATOM | 519 | CD1 | PHE | A. | 53 | 154.039 | 35.311 | 10.691 | 1.00 | 31.58 |
| ATOM | 520 | CD2 | PHE | A | 53 | 154.912 | 33.092 | 10.682 | 1.00 | 30.53 |
| ATOM | 521 | CE1 | PHE | A | 53 | 154.759 | 35.603 | 9.545 | 1.00 | 30.85 |
| ATOM | 522 | CE2 | PHE | A | 53 | 155.637 | 33.373 | 9.533 | 1.00 | 30.45 |
| ATOM | 523 | CZ | PHE | A | 53 | 155.559 | 34.631 | 8.964 | 1.00 | 30.49 |
| ATOM | 524 | C | PHE | A | 53 | 153.071 | 33.965 | 15.006 | 1.00 | 35.40 |
| ATOM | 525 | O | PHE | A | 53 | 152.746 | 32.814 | 15.276 | 1.00 | 34.75 |
| ATOM | 526 | N | TYR | A | 54 | 152.720 | 34.998 | 15.757 | 1.00 | 34.80 |
| ATOM | 528 | CA | TYR | A | 54 | 151.960 | 34.798 | 16.974 | 1.00 | 37.29 |
| ATOM | 529 | CB | TYR | A | 54 | 152.947 | 34.533 | 18.103 | 1.00 | 39.38 |
| ATOM | 530 | CG | TYR | A | 54 | 153.841 | 35.721 | 18.375 | 1.00 | 42.01 |
| ATOM | 531 | CD1 | TYR | A | 54 | 154.782 | 36.139 | 17.437 | 1.00 | 42.10 |
| ATOM | 532 | CE1 | TYR | A | 54 | 155.552 | 37.270 | 17.649 | 1.00 | 40.85 |
| ATOM | 533 | CD2 | TYR | A | 54 | 153.703 | 36.467 | 19.541 | 1.00 | 43.83 |
| ATOM | 534 | CE2 | TYR | A | 54 | 154.472 | 37.600 | 19.763 | 1.00 | 43.33 |
| ATOM | 535 | CZ | TYR | A | 54 | 155.391 | 37.995 | 18.811 | 1.00 | 42.17 |
| ATOM | 536 | OH | TYR | A | 54 | 156.139 | 39.126 | 19.018 | 1.00 | 46.33 |
| ATOM | 538 | C | TYR | A | 54 | 151.100 | 35.990 | 17.362 | 1.00 | 38.69 |
| ATOM | 539 | O | TYR | A | 54 | 151.072 | 37.007 | 16.672 | 1.00 | 41.66 |
| ATOM | 540 | N | SER | A | 55 | 150.430 | 35.858 | 18.505 | 1.00 | 40.04 |
| ATOM | 542 | CA | SER | A | 55 | 149.562 | 36.894 | 19.056 | 1.00 | 40.41 |
| ATOM | 543 | CB | SER | A | 55 | 150.407 | 38.091 | 19.499 | 1.00 | 41.89 |
| ATOM | 544 | OG | SER | A | 55 | 149.708 | 38.923 | 20.409 | 1.00 | 46.68 |
| ATOM | 546 | C | SER | A | 55 | 148.489 | 37.318 | 18.051 | 1.00 | 41.35 |
| ATOM | 547 | O | SER | A | 55 | 148.115 | 38.492 | 17.973 | 1.00 | 40.80 |
| ATOM | 548 | N | MET | A | 56 | 147.984 | 36.346 | 17.299 | 1.00 | 42.39 |
| ATOM | 550 | CA | MET | A | 56 | 146.968 | 36.606 | 16.292 | 1.00 | 43.64 |
| ATOM | 551 | CB | MET | A | 56 | 146.996 | 35.513 | 15.225 | 1.00 | 42.07 |
| ATOM | 552 | CG | MET | A | 56 | 148.310 | 35.414 | 14.475 | 1.00 | 39.09 |
| ATOM | 553 | SD | MET | A | 56 | 148.335 | 34.056 | 13.289 | 1.00 | 41.60 |
| ATOM | 554 | CE | MET | A | 56 | 149.266 | 32.847 | 14.209 | 1.00 | 36.68 |
| ATOM | 555 | C | MET | A | 56 | 145.580 | 36.708 | 16.910 | 1.00 | 46.06 |
| ATOM | 556 | O | MET | A | 56 | 145.222 | 35.928 | 17.794 | 1.00 | 48.27 |
| ATOM | 557 | N | ASP | A | 57 | 144.820 | 37.700 | 16.463 | 1.00 | 47.20 |
| ATOM | 559 | CA | ASP | A | 57 | 143.466 | 37.930 | 16.941 | 1.00 | 47.94 |
| ATOM | 560 | CB | ASP | A | 57 | 143.408 | 39.167 | 17.848 | 1.00 | 52.70 |
| ATOM | 561 | CG | ASP | A | 57 | 143.578 | 38.831 | 19.324 | 1.00 | 58.73 |
| ATOM | 562 | OD1 | ASP | A | 57 | 142.552 | 38.587 | 19.999 | 1.00 | 62.99 |
| ATOM | 563 | OD2 | ASP | A | 57 | 144.729 | 38.835 | 19.818 | 1.00 | 59.77 |
| ATOM | 564 | C | ASP | A | 57 | 142.566 | 38.151 | 15.736 | 1.00 | 46.86 |
| ATOM | 565 | O | ASP | A | 57 | 143.001 | 38.684 | 14.712 | 1.00 | 44.98 |
| ATOM | 566 | N | ILE | A | 58 | 141.324 | 37.699 | 15.843 | 1.00 | 45.46 |
| ATOM | 568 | CA | ILE | A | 58 | 140.356 | 37.872 | 14.773 | 1.00 | 45.28 |
| ATOM | 569 | CB | ILE | A | 58 | 139.232 | 36.822 | 14.869 | 1.00 | 42.99 |
| ATOM | 570 | CG2 | ILE | A | 58 | 138.200 | 37.052 | 13.782 | 1.00 | 41.93 |
| ATOM | 571 | CG1 | ILE | A | 58 | 139.825 | 35.415 | 14.744 | 1.00 | 42.15 |
| ATOM | 572 | CD1 | ILE | A | 58 | 138.803 | 34.306 | 14.802 | 1.00 | 41.09 |
| ATOM | 573 | C | ILE | A | 58 | 139.783 | 39.277 | 14.936 | 1.00 | 47.07 |
| ATOM | 574 | O | ILE | A | 58 | 139.271 | 39.615 | 15.999 | 1.00 | 48.46 |
| ATOM | 575 | N | ARG | A | 59 | 139.945 | 40.116 | 13.918 | 1.00 | 49.05 |
| ATOM | 577 | CA | ARG | A | 59 | 139.441 | 41.486 | 13.973 | 1.00 | 51.28 |
| ATOM | 578 | CB | ARG | A | 59 | 140.435 | 42.459 | 13.326 | 1.00 | 53.21 |
| ATOM | 579 | CG | ARG | A | 59 | 141.760 | 42.590 | 14.062 | 1.00 | 55.13 |
| ATOM | 580 | CD | ARG | A | 59 | 141.578 | 43.175 | 15.453 | 1.00 | 59.25 |
| ATOM | 581 | NE | ARG | A | 59 | 142.833 | 43.178 | 16.202 | 1.00 | 63.13 |
| ATOM | 583 | CZ | ARG | A | 59 | 142.981 | 43.681 | 17.424 | 1.00 | 66.00 |
| ATOM | 584 | NH1 | ARG | A | 59 | 141.950 | 44.231 | 18.054 | 1.00 | 68.47 |
| ATOM | 587 | NH2 | ARG | A | 59 | 144.164 | 43.631 | 18.022 | 1.00 | 67.15 |
| ATOM | 590 | C | ARG | A | 59 | 138.066 | 41.627 | 13.322 | 1.00 | 52.12 |
| ATOM | 591 | O | ARG | A | 59 | 137.256 | 42.456 | 13.746 | 1.00 | 54.45 |
| ATOM | 592 | N | GLU | A | 60 | 137.823 | 40.857 | 12.265 | 1.00 | 52.91 |
| ATOM | 594 | CA | GLU | A | 60 | 136.538 | 40.878 | 11.570 | 1.00 | 52.94 |
| ATOM | 595 | CB | GLU | A | 60 | 136.492 | 41.944 | 10.476 | 1.00 | 55.28 |
| ATOM | 596 | CG | GLU | A | 60 | 135.133 | 41.994 | 9.776 | 1.00 | 60.74 |
| ATOM | 597 | CD | GLU | A | 60 | 135.131 | 42.841 | 8.523 | 1.00 | 64.33 |
| ATOM | 598 | OE1 | GLU | A | 60 | 135.450 | 44.046 | 8.618 | 1.00 | 67.37 |
| ATOM | 599 | OE2 | GLU | A | 60 | 134.798 | 42.300 | 7.443 | 1.00 | 65.16 |
| ATOM | 600 | C | GLU | A | 60 | 136.279 | 39.518 | 10.948 | 1.00 | 51.42 |
| ATOM | 601 | O | GLU | A | 60 | 137.170 | 38.930 | 10.342 | 1.00 | 52.44 |
| ATOM | 602 | N | PHE | A | 61 | 135.052 | 39.035 | 11.093 | 1.00 | 50.66 |
| ATOM | 604 | CA | PHE | A | 61 | 134.646 | 37.741 | 10.558 | 1.00 | 49.14 |
| ATOM | 605 | CB | PHE | A | 61 | 134.536 | 36.736 | 11.707 | 1.00 | 44.10 |
| ATOM | 606 | CG | PHE | A | 61 | 134.679 | 35.308 | 11.289 | 1.00 | 37.41 |
| ATOM | 607 | CD1 | PHE | A | 61 | 133.998 | 34.814 | 10.188 | 1.00 | 36.68 |
| ATOM | 608 | CD2 | PHE | A | 61 | 135.486 | 34.448 | 12.018 | 1.00 | 34.73 |
| ATOM | 609 | CE1 | PHE | A | 61 | 134.116 | 33.477 | 9.817 | 1.00 | 36.53 |
| ATOM | 610 | CE2 | PHE | A | 61 | 135.612 | 33.115 | 11.658 | 1.00 | 34.86 |
| ATOM | 611 | CZ | PHE | A | 61 | 134.922 | 32.627 | 10.553 | 1.00 | 35.68 |
| ATOM | 612 | C | PHE | A | 61 | 133.278 | 37.962 | 9.924 | 1.00 | 50.95 |
| ATOM | 613 | O | PHE | A | 61 | 132.249 | 37.646 | 10.521 | 1.00 | 52.86 |
| ATOM | 614 | N | GLN | A | 62 | 133.259 | 38.540 | 8.729 | 1.00 | 51.57 |
| ATOM | 616 | CA | GLN | A | 62 | 131.993 | 38.813 | 8.080 | 1.00 | 51.07 |
| ATOM | 617 | CB | GLN | A | 62 | 132.097 | 39.993 | 7.120 | 1.00 | 54.59 |
| ATOM | 618 | CG | GLN | A | 62 | 130.727 | 40.463 | 6.639 | 1.00 | 62.31 |
| ATOM | 619 | CD | GLN | A | 62 | 130.775 | 41.665 | 5.708 | 1.00 | 67.35 |
| ATOM | 620 | OE1 | GLN | A | 62 | 129.812 | 41.935 | 4.991 | 1.00 | 69.86 |
| ATOM | 621 | NE2 | GLN | A | 62 | 131.884 | 42.401 | 5.727 | 1.00 | 71.01 |
| ATOM | 624 | C | GLN | A | 62 | 131.391 | 37.625 | 7.367 | 1.00 | 48.52 |
| ATOM | 625 | O | GLN | A | 62 | 132.065 | 36.912 | 6.629 | 1.00 | 48.13 |
| ATOM | 626 | N | LEU | A | 63 | 130.107 | 37.422 | 7.622 | 1.00 | 48.11 |
| ATOM | 628 | CA | LEU | A | 63 | 129.324 | 36.358 | 7.017 | 1.00 | 47.47 |
| ATOM | 629 | CB | LEU | A | 63 | 128.753 | 35.438 | 8.103 | 1.00 | 44.43 |
| ATOM | 630 | CG | LEU | A | 63 | 129.754 | 34.759 | 9.044 | 1.00 | 42.13 |
| ATOM | 631 | CD1 | LEU | A | 63 | 129.021 | 34.080 | 10.181 | 1.00 | 42.20 |
| ATOM | 632 | CD2 | LEU | A | 63 | 130.593 | 33.752 | 8.282 | 1.00 | 40.96 |
| ATOM | 633 | C | LEU | A | 63 | 128.208 | 37.122 | 6.314 | 1.00 | 48.35 |
| ATOM | 634 | O | LEU | A | 63 | 127.138 | 37.345 | 6.882 | 1.00 | 49.30 |
| ATOM | 635 | N | PRO | A | 64 | 128.471 | 37.585 | 5.082 | 1.00 | 49.75 |
| ATOM | 636 | CD | PRO | A | 64 | 129.686 | 37.302 | 4.297 | 1.00 | 50.27 |
| ATOM | 637 | CA | PRO | A | 64 | 127.508 | 38.347 | 4.283 | 1.00 | 50.73 |
| ATOM | 638 | CB | PRO | A | 64 | 128.309 | 38.687 | 3.026 | 1.00 | 50.48 |
| ATOM | 639 | CG | PRO | A | 64 | 129.210 | 37.513 | 2.878 | 1.00 | 49.49 |
| ATOM | 640 | C | PRO | A | 64 | 126.219 | 37.616 | 3.937 | 1.00 | 51.98 |
| ATOM | 641 | O | PRO | A | 64 | 125.129 | 38.167 | 4.083 | 1.00 | 54.28 |
| ATOM | 642 | N | SER | A | 65 | 126.341 | 36.375 | 3.488 | 1.00 | 51.59 |
| ATOM | 644 | CA | SER | A | 65 | 125.177 | 35.607 | 3.101 | 1.00 | 50.95 |
| ATOM | 645 | CB | SER | A | 65 | 125.217 | 35.356 | 1.599 | 1.00 | 52.70 |
| ATOM | 646 | OG | SER | A | 65 | 126.503 | 34.910 | 1.205 | 1.00 | 55.07 |
| ATOM | 648 | C | SER | A | 65 | 125.053 | 34.288 | 3.832 | 1.00 | 50.87 |
| ATOM | 649 | O | SER | A | 65 | 126.052 | 33.627 | 4.120 | 1.00 | 52.05 |
| ATOM | 650 | N | SER | A | 66 | 123.811 | 33.928 | 4.136 | 1.00 | 50.53 |
| ATOM | 652 | CA | SER | A | 66 | 123.477 | 32.680 | 4.805 | 1.00 | 48.85 |
| ATOM | 653 | CB | SER | A | 66 | 123.322 | 32.878 | 6.312 | 1.00 | 48.24 |
| ATOM | 654 | OG | SER | A | 66 | 122.307 | 33.820 | 6.615 | 1.00 | 49.82 |
| ATOM | 656 | C | SER | A | 66 | 122.154 | 32.244 | 4.202 | 1.00 | 49.43 |
| ATOM | 657 | O | SER | A | 66 | 121.365 | 33.080 | 3.745 | 1.00 | 48.59 |
| ATOM | 658 | N | GLN | A | 67 | 121.925 | 30.940 | 4.165 | 1.00 | 49.88 |
| ATOM | 660 | CA | GLN | A | 67 | 120.696 | 30.409 | 3.605 | 1.00 | 49.90 |
| ATOM | 661 | CB | GLN | A | 67 | 120.831 | 30.267 | 2.086 | 1.00 | 52.61 |
| ATOM | 662 | CG | GLN | A | 67 | 121.928 | 29.308 | 1.632 | 1.00 | 58.79 |
| ATOM | 663 | CD | GLN | A | 67 | 121.984 | 29.154 | 0.121 | 1.00 | 62.57 |
| ATOM | 664 | OE1 | GLN | A | 67 | 122.550 | 29.997 | −0.576 | 1.00 | 66.32 |
| ATOM | 665 | NE2 | GLN | A | 67 | 121.407 | 28.073 | −0.392 | 1.00 | 62.89 |
| ATOM | 668 | C | GLN | A | 67 | 120.337 | 29.065 | 4.221 | 1.00 | 48.02 |
| ATOM | 669 | O | GLN | A | 67 | 121.215 | 28.306 | 4.635 | 1.00 | 49.44 |
| ATOM | 670 | N | ILE | A | 68 | 119.040 | 28.803 | 4.328 | 1.00 | 45.23 |
| ATOM | 672 | CA | ILE | A | 68 | 118.551 | 27.544 | 4.864 | 1.00 | 42.63 |
| ATOM | 673 | CB | ILE | A | 68 | 117.826 | 27.721 | 6.220 | 1.00 | 44.01 |
| ATOM | 674 | CG2 | ILE | A | 68 | 116.977 | 26.499 | 6.546 | 1.00 | 45.24 |
| ATOM | 675 | CG1 | ILE | A | 68 | 118.850 | 27.925 | 7.333 | 1.00 | 44.56 |
| ATOM | 676 | CD1 | ILE | A | 68 | 118.260 | 27.851 | 8.719 | 1.00 | 47.20 |
| ATOM | 677 | C | ILE | A | 68 | 117.613 | 26.935 | 3.835 | 1.00 | 40.01 |
| ATOM | 678 | O | ILE | A | 68 | 116.560 | 27.491 | 3.524 | 1.00 | 41.30 |
| ATOM | 679 | N | SER | A | 69 | 118.051 | 25.833 | 3.250 | 1.00 | 37.17 |
| ATOM | 681 | CA | SER | A | 69 | 117.264 | 25.140 | 2.258 | 1.00 | 34.46 |
| ATOM | 682 | CB | SER | A | 69 | 118.165 | 24.600 | 1.147 | 1.00 | 34.89 |
| ATOM | 683 | OG | SER | A | 69 | 118.890 | 25.640 | 0.511 | 1.00 | 40.63 |
| ATOM | 685 | C | SER | A | 69 | 116.570 | 23.984 | 2.939 | 1.00 | 33.71 |
| ATOM | 686 | O | SER | A | 69 | 117.085 | 23.414 | 3.896 | 1.00 | 34.03 |
| ATOM | 687 | N | MET | A | 70 | 115.405 | 23.625 | 2.430 | 1.00 | 33.11 |
| ATOM | 689 | CA | MET | A | 70 | 114.659 | 22.519 | 2.981 | 1.00 | 34.74 |
| ATOM | 690 | CB | MET | A | 70 | 113.166 | 22.841 | 3.012 | 1.00 | 36.59 |
| ATOM | 691 | CG | MET | A | 70 | 112.794 | 23.880 | 4.051 | 1.00 | 41.38 |
| ATOM | 692 | SD | MET | A | 70 | 111.050 | 24.288 | 4.028 | 1.00 | 47.62 |
| ATOM | 693 | CE | MET | A | 70 | 111.101 | 25.834 | 3.173 | 1.00 | 47.72 |
| ATOM | 694 | C | MET | A | 70 | 114.906 | 21.274 | 2.153 | 1.00 | 35.74 |
| ATOM | 695 | O | MET | A | 70 | 114.752 | 21.279 | 0.928 | 1.00 | 37.33 |
| ATOM | 696 | N | VAL | A | 71 | 115.366 | 20.226 | 2.820 | 1.00 | 35.02 |
| ATOM | 698 | CA | VAL | A | 71 | 115.613 | 18.957 | 2.165 | 1.00 | 32.82 |
| ATOM | 699 | CB | VAL | A | 71 | 116.938 | 18.343 | 2.637 | 1.00 | 30.47 |
| ATOM | 700 | CG1 | VAL | A | 71 | 117.206 | 17.049 | 1.905 | 1.00 | 28.06 |
| ATOM | 701 | CG2 | VAL | A | 71 | 118.069 | 19.324 | 2.404 | 1.00 | 28.88 |
| ATOM | 702 | C | VAL | A | 71 | 114.438 | 18.072 | 2.571 | 1.00 | 33.73 |
| ATOM | 703 | O | VAL | A | 71 | 114.430 | 17.505 | 3.664 | 1.00 | 35.31 |
| ATOM | 704 | N | PRO | A | 72 | 113.411 | 17.975 | 1.708 | 1.00 | 33.98 |
| ATOM | 705 | CD | PRO | A | 72 | 113.402 | 18.500 | 0.327 | 1.00 | 33.44 |
| ATOM | 706 | CA | PRO | A | 72 | 112.211 | 17.168 | 1.965 | 1.00 | 34.72 |
| ATOM | 707 | CB | PRO | A | 72 | 111.660 | 16.936 | 0.562 | 1.00 | 33.90 |
| ATOM | 708 | CG | PRO | A | 72 | 111.980 | 18.231 | −0.124 | 1.00 | 32.49 |
| ATOM | 709 | C | PRO | A | 72 | 112.495 | 15.857 | 2.695 | 1.00 | 36.34 |
| ATOM | 710 | O | PRO | A | 72 | 113.417 | 15.131 | 2.340 | 1.00 | 39.70 |
| ATOM | 711 | N | ASN | A | 73 | 111.745 | 15.607 | 3.765 | 1.00 | 38.08 |
| ATOM | 713 | CA | ASN | A | 73 | 111.886 | 14.399 | 4.580 | 1.00 | 39.44 |
| ATOM | 714 | CB | ASN | A | 73 | 111.553 | 13.146 | 3.768 | 1.00 | 45.34 |
| ATOM | 715 | CG | ASN | A | 73 | 110.071 | 12.875 | 3.699 | 1.00 | 50.49 |
| ATOM | 716 | OD1 | ASN | A | 73 | 109.392 | 13.317 | 2.770 | 1.00 | 53.54 |
| ATOM | 717 | ND2 | ASN | A | 73 | 109.552 | 12.149 | 4.690 | 1.00 | 53.95 |
| ATOM | 720 | C | ASN | A | 73 | 113.231 | 14.192 | 5.252 | 1.00 | 38.00 |
| ATOM | 721 | O | ASN | A | 73 | 113.408 | 13.204 | 5.962 | 1.00 | 37.89 |
| ATOM | 722 | N | VAL | A | 74 | 114.165 | 15.116 | 5.050 | 1.00 | 37.67 |
| ATOM | 724 | CA | VAL | A | 74 | 115.498 | 14.995 | 5.632 | 1.00 | 36.66 |
| ATOM | 725 | CB | VAL | A | 74 | 116.598 | 15.041 | 4.525 | 1.00 | 37.47 |
| ATOM | 726 | CG1 | VAL | A | 74 | 117.976 | 14.753 | 5.118 | 1.00 | 35.10 |
| ATOM | 727 | CG2 | VAL | A | 74 | 116.280 | 14.046 | 3.402 | 1.00 | 33.69 |
| ATOM | 728 | C | VAL | A | 74 | 115.784 | 16.054 | 6.704 | 1.00 | 37.30 |
| ATOM | 729 | O | VAL | A | 74 | 116.106 | 15.717 | 7.848 | 1.00 | 39.11 |
| ATOM | 730 | N | GLY | A | 75 | 115.672 | 17.328 | 6.342 | 1.00 | 35.75 |
| ATOM | 732 | CA | GLY | A | 75 | 115.928 | 18.384 | 7.306 | 1.00 | 33.77 |
| ATOM | 733 | C | GLY | A | 75 | 116.279 | 19.698 | 6.647 | 1.00 | 33.37 |
| ATOM | 734 | O | GLY | A | 75 | 115.827 | 19.981 | 5.536 | 1.00 | 32.85 |
| ATOM | 735 | N | LEU | A | 76 | 117.075 | 20.509 | 7.338 | 1.00 | 34.22 |
| ATOM | 737 | CA | LEU | A | 76 | 117.504 | 21.811 | 6.828 | 1.00 | 33.33 |
| ATOM | 738 | CB | LEU | A | 76 | 117.329 | 22.894 | 7.897 | 1.00 | 30.43 |
| ATOM | 739 | CG | LEU | A | 76 | 115.958 | 23.105 | 8.524 | 1.00 | 28.67 |
| ATOM | 740 | CD1 | LEU | A | 76 | 116.081 | 24.069 | 9.683 | 1.00 | 29.06 |
| ATOM | 741 | CD2 | LEU | A | 76 | 114.995 | 23.633 | 7.481 | 1.00 | 31.34 |
| ATOM | 742 | C | LEU | A | 76 | 118.979 | 21.755 | 6.448 | 1.00 | 33.33 |
| ATOM | 743 | O | LEU | A | 76 | 119.736 | 20.942 | 6.967 | 1.00 | 34.06 |
| ATOM | 744 | N | LYS | A | 77 | 119.383 | 22.636 | 5.550 | 1.00 | 34.34 |
| ATOM | 746 | CA | LYS | A | 77 | 120.766 | 22.711 | 5.131 | 1.00 | 33.56 |
| ATOM | 747 | CB | LYS | A | 77 | 120.927 | 22.201 | 3.700 | 1.00 | 35.32 |
| ATOM | 748 | CG | LYS | A | 77 | 122.345 | 22.283 | 3.166 | 1.00 | 38.99 |
| ATOM | 749 | CD | LYS | A | 77 | 122.450 | 21.670 | 1.782 | 1.00 | 41.42 |
| ATOM | 750 | CE | LYS | A | 77 | 122.388 | 20.150 | 1.838 | 1.00 | 41.64 |
| ATOM | 751 | NZ | LYS | A | 77 | 123.630 | 19.565 | 2.421 | 1.00 | 43.94 |
| ATOM | 755 | C | LYS | A | 77 | 121.131 | 24.175 | 5.228 | 1.00 | 34.11 |
| ATOM | 756 | O | LYS | A | 77 | 120.556 | 25.013 | 4.538 | 1.00 | 34.01 |
| ATOM | 757 | N | PHE | A | 78 | 122.009 | 24.478 | 6.173 | 1.00 | 36.93 |
| ATOM | 759 | CA | PHE | A | 78 | 122.482 | 25.830 | 6.427 | 1.00 | 37.23 |
| ATOM | 760 | CB | PHE | A | 78 | 122.720 | 25.983 | 7.933 | 1.00 | 37.11 |
| ATOM | 761 | CG | PHE | A | 78 | 123.233 | 27.329 | 8.340 | 1.00 | 36.19 |
| ATOM | 762 | CD1 | PHE | A | 78 | 122.536 | 28.481 | 8.016 | 1.00 | 35.10 |
| ATOM | 763 | CD2 | PHE | A | 78 | 124.415 | 27.442 | 9.059 | 1.00 | 36.97 |
| ATOM | 764 | CE1 | PHE | A | 78 | 123.008 | 29.725 | 8.400 | 1.00 | 33.97 |
| ATOM | 765 | CE2 | PHE | A | 78 | 124.892 | 28.679 | 9.446 | 1.00 | 36.53 |
| ATOM | 766 | CZ | PHE | A | 78 | 124.185 | 29.824 | 9.115 | 1.00 | 36.23 |
| ATOM | 767 | C | PHE | A | 78 | 123.780 | 26.063 | 5.646 | 1.00 | 38.21 |
| ATOM | 768 | O | PHE | A | 78 | 124.706 | 25.256 | 5.731 | 1.00 | 37.35 |
| ATOM | 769 | N | SER | A | 79 | 123.845 | 27.160 | 4.892 | 1.00 | 39.48 |
| ATOM | 771 | CA | SER | A | 79 | 125.028 | 27.489 | 4.097 | 1.00 | 40.20 |
| ATOM | 772 | CB | SER | A | 79 | 124.802 | 27.126 | 2.625 | 1.00 | 41.51 |
| ATOM | 773 | OG | SER | A | 79 | 124.408 | 25.773 | 2.464 | 1.00 | 48.40 |
| ATOM | 775 | C | SER | A | 79 | 125.416 | 28.963 | 4.160 | 1.00 | 40.10 |
| ATOM | 776 | O | SER | A | 79 | 124.557 | 29.842 | 4.112 | 1.00 | 39.14 |
| ATOM | 777 | N | ILE | A | 80 | 126.713 | 29.219 | 4.297 | 1.00 | 40.97 |
| ATOM | 779 | CA | ILE | A | 80 | 127.263 | 30.575 | 4.309 | 1.00 | 41.40 |
| ATOM | 780 | CB | ILE | A | 80 | 128.002 | 30.885 | 5.612 | 1.00 | 39.45 |
| ATOM | 781 | CG2 | ILE | A | 80 | 128.518 | 32.307 | 5.583 | 1.00 | 40.67 |
| ATOM | 782 | CG1 | ILE | A | 80 | 127.057 | 30.714 | 6.797 | 1.00 | 38.41 |
| ATOM | 783 | CD1 | ILE | A | 80 | 127.690 | 31.022 | 8.120 | 1.00 | 39.45 |
| ATOM | 784 | C | ILE | A | 80 | 128.246 | 30.524 | 3.143 | 1.00 | 43.93 |
| ATOM | 785 | O | ILE | A | 80 | 128.944 | 29.519 | 2.976 | 1.00 | 45.50 |
| ATOM | 786 | N | SER | A | 81 | 128.327 | 31.585 | 2.344 | 1.00 | 47.35 |
| ATOM | 788 | CA | SER | A | 81 | 129.193 | 31.541 | 1.168 | 1.00 | 49.22 |
| ATOM | 789 | CB | SER | A | 81 | 128.338 | 31.613 | −0.097 | 1.00 | 49.49 |
| ATOM | 790 | OG | SER | A | 81 | 127.332 | 32.602 | 0.032 | 1.00 | 52.43 |
| ATOM | 792 | C | SER | A | 81 | 130.412 | 32.432 | 1.004 | 1.00 | 50.65 |
| ATOM | 793 | O | SER | A | 81 | 131.482 | 31.937 | 0.658 | 1.00 | 52.88 |
| ATOM | 794 | N | ASN | A | 82 | 130.276 | 33.738 | 1.185 | 1.00 | 52.50 |
| ATOM | 796 | CA | ASN | A | 82 | 131.442 | 34.599 | 0.995 | 1.00 | 54.71 |
| ATOM | 797 | CB | ASN | A | 82 | 131.104 | 35.767 | 0.061 | 1.00 | 58.34 |
| ATOM | 798 | CG | ASN | A | 82 | 132.342 | 36.391 | −0.570 | 1.00 | 61.43 |
| ATOM | 799 | OD1 | ASN | A | 82 | 132.462 | 37.613 | −0.657 | 1.00 | 63.94 |
| ATOM | 800 | ND2 | ASN | A | 82 | 133.257 | 35.550 | −1.039 | 1.00 | 61.92 |
| ATOM | 803 | C | ASN | A | 82 | 132.040 | 35.098 | 2.303 | 1.00 | 54.00 |
| ATOM | 804 | O | ASN | A | 82 | 132.268 | 36.297 | 2.482 | 1.00 | 54.15 |
| ATOM | 805 | N | ALA | A | 83 | 132.325 | 34.162 | 3.205 | 1.00 | 53.37 |
| ATOM | 807 | CA | ALA | A | 83 | 132.892 | 34.501 | 4.506 | 1.00 | 52.11 |
| ATOM | 808 | CB | ALA | A | 83 | 132.952 | 33.273 | 5.402 | 1.00 | 51.81 |
| ATOM | 809 | C | ALA | A | 83 | 134.272 | 35.131 | 4.359 | 1.00 | 50.64 |
| ATOM | 810 | O | ALA | A | 83 | 135.124 | 34.640 | 3.610 | 1.00 | 49.58 |
| ATOM | 811 | N | ASN | A | 84 | 134.467 | 36.228 | 5.078 | 1.00 | 48.85 |
| ATOM | 813 | CA | ASN | A | 84 | 135.706 | 36.983 | 5.063 | 1.00 | 47.97 |
| ATOM | 814 | CB | ASN | A | 84 | 135.423 | 38.393 | 4.536 | 1.00 | 51.32 |
| ATOM | 815 | CG | ASN | A | 84 | 136.560 | 39.361 | 4.793 | 1.00 | 56.05 |
| ATOM | 816 | OD1 | ASN | A | 84 | 137.470 | 39.499 | 3.978 | 1.00 | 61.24 |
| ATOM | 817 | ND2 | ASN | A | 84 | 136.500 | 40.060 | 5.919 | 1.00 | 56.91 |
| ATOM | 820 | C | ASN | A | 84 | 136.243 | 37.042 | 6.485 | 1.00 | 45.82 |
| ATOM | 821 | O | ASN | A | 84 | 135.601 | 37.606 | 7.368 | 1.00 | 47.29 |
| ATOM | 822 | N | ILE | A | 85 | 137.405 | 36.438 | 6.703 | 1.00 | 43.85 |
| ATOM | 824 | CA | ILE | A | 85 | 138.052 | 36.402 | 8.014 | 1.00 | 41.41 |
| ATOM | 825 | CB | ILE | A | 85 | 138.495 | 34.952 | 8.386 | 1.00 | 35.61 |
| ATOM | 826 | CG2 | ILE | A | 85 | 139.011 | 34.901 | 9.804 | 1.00 | 32.01 |
| ATOM | 827 | CG1 | ILE | A | 85 | 137.342 | 33.965 | 8.222 | 1.00 | 30.72 |
| ATOM | 828 | CD1 | ILE | A | 85 | 137.712 | 32.557 | 8.587 | 1.00 | 25.79 |
| ATOM | 829 | C | ILE | A | 85 | 139.315 | 37.272 | 8.017 | 1.00 | 43.73 |
| ATOM | 830 | O | ILE | A | 85 | 140.343 | 36.873 | 7.471 | 1.00 | 47.66 |
| ATOM | 831 | N | LYS | A | 86 | 139.239 | 38.471 | 8.578 | 1.00 | 42.83 |
| ATOM | 833 | CA | LYS | A | 86 | 140.420 | 39.313 | 8.645 | 1.00 | 43.65 |
| ATOM | 834 | CB | LYS | A | 86 | 140.090 | 40.779 | 8.363 | 1.00 | 47.39 |
| ATOM | 835 | CG | LYS | A | 86 | 139.895 | 41.055 | 6.873 | 1.00 | 54.31 |
| ATOM | 836 | CD | LYS | A | 86 | 140.065 | 42.528 | 6.509 | 1.00 | 58.05 |
| ATOM | 837 | CE | LYS | A | 86 | 138.937 | 43.394 | 7.045 | 1.00 | 61.18 |
| ATOM | 838 | NZ | LYS | A | 86 | 139.107 | 44.820 | 6.642 | 1.00 | 64.32 |
| ATOM | 842 | C | LYS | A | 86 | 141.100 | 39.135 | 9.996 | 1.00 | 43.61 |
| ATOM | 843 | O | LYS | A | 86 | 140.514 | 39.404 | 11.043 | 1.00 | 42.95 |
| ATOM | 844 | N | ILE | A | 87 | 142.327 | 38.627 | 9.961 | 1.00 | 43.89 |
| ATOM | 846 | CA | ILE | A | 87 | 143.104 | 38.373 | 11.165 | 1.00 | 44.27 |
| ATOM | 847 | CB | ILE | A | 87 | 143.568 | 36.895 | 11.206 | 1.00 | 43.55 |
| ATOM | 848 | CG2 | ILE | A | 87 | 144.365 | 36.609 | 12.473 | 1.00 | 42.71 |
| ATOM | 849 | CG1 | ILE | A | 87 | 142.358 | 35.964 | 11.129 | 1.00 | 41.65 |
| ATOM | 850 | CD1 | ILE | A | 87 | 142.723 | 34.504 | 11.044 | 1.00 | 42.75 |
| ATOM | 851 | C | ILE | A | 87 | 144.327 | 39.291 | 11.212 | 1.00 | 44.82 |
| ATOM | 852 | O | ILE | A | 87 | 144.817 | 39.745 | 10.176 | 1.00 | 46.46 |
| ATOM | 853 | N | SER | A | 88 | 144.805 | 39.568 | 12.417 | 1.00 | 43.43 |
| ATOM | 855 | CA | SER | A | 88 | 145.970 | 40.414 | 12.606 | 1.00 | 44.37 |
| ATOM | 856 | CB | SER | A | 88 | 145.548 | 41.825 | 13.027 | 1.00 | 46.30 |
| ATOM | 857 | OG | ser | a | 88 | 144.757 | 42.451 | 12.029 | 1.00 | 50.16 |
| ATOM | 859 | C | SER | A | 88 | 146.830 | 39.795 | 13.690 | 1.00 | 43.85 |
| ATOM | 860 | O | SER | A | 88 | 146.327 | 39.073 | 14.549 | 1.00 | 43.41 |
| ATOM | 861 | N | GLY | A | 89 | 148.128 | 40.061 | 13.638 | 1.00 | 45.04 |
| ATOM | 863 | CA | GLY | A | 89 | 149.029 | 39.525 | 14.640 | 1.00 | 47.03 |
| ATOM | 864 | C | GLY | A | 89 | 150.415 | 40.123 | 14.532 | 1.00 | 47.50 |
| ATOM | 865 | O | GLY | A | 89 | 150.611 | 41.140 | 13.861 | 1.00 | 47.81 |
| ATOM | 866 | N | LYS | A | 90 | 151.377 | 39.497 | 15.200 | 1.00 | 47.62 |
| ATOM | 868 | CA | LYS | A | 90 | 152.759 | 39.957 | 15.176 | 1.00 | 47.17 |
| ATOM | 869 | CB | LYS | A | 90 | 153.197 | 40.427 | 16.567 | 1.00 | 47.20 |
| ATOM | 870 | CG | LYS | A | 90 | 152.328 | 41.514 | 17.189 | 1.00 | 49.57 |
| ATOM | 871 | CD | LYS | A | 90 | 152.708 | 41.723 | 18.651 | 1.00 | 53.99 |
| ATOM | 872 | CE | LYS | A | 90 | 151.725 | 42.625 | 19.391 | 1.00 | 57.45 |
| ATOM | 873 | NZ | LYS | A | 90 | 152.042 | 42.735 | 20.855 | 1.00 | 59.91 |
| ATOM | 877 | C | LYS | A | 90 | 153.629 | 38.787 | 14.738 | 1.00 | 45.69 |
| ATOM | 878 | O | LYS | A | 90 | 153.171 | 37.644 | 14.697 | 1.00 | 45.87 |
| ATOM | 879 | N | TRP | A | 91 | 154.874 | 39.078 | 14.389 | 1.00 | 44.12 |
| ATOM | 881 | CA | TRP | A | 91 | 155.817 | 38.052 | 13.969 | 1.00 | 43.19 |
| ATOM | 882 | CB | TRP | A | 91 | 155.734 | 37.824 | 12.456 | 1.00 | 39.90 |
| ATOM | 883 | CG | TRP | A | 91 | 156.078 | 39.038 | 11.694 | 1.00 | 37.85 |
| ATOM | 884 | CD2 | TRP | A | 91 | 157.390 | 39.466 | 11.313 | 1.00 | 39.20 |
| ATOM | 885 | CE2 | TRP | A | 91 | 157.251 | 40.722 | 10.682 | 1.00 | 39.17 |
| ATOM | 886 | CE3 | TRP | A | 91 | 158.672 | 38.918 | 11.451 | 1.00 | 36.54 |
| ATOM | 887 | CD1 | TRP | A | 91 | 155.222 | 40.012 | 11.286 | 1.00 | 39.46 |
| ATOM | 888 | NE1 | TRP | A | 91 | 155.916 | 41.032 | 10.681 | 1.00 | 39.30 |
| ATOM | 890 | CZ2 | TRP | A | 91 | 158.344 | 41.437 | 10.188 | 1.00 | 38.03 |
| ATOM | 891 | CZ3 | TRP | A | 91 | 159.757 | 39.628 | 10.963 | 1.00 | 35.59 |
| ATOM | 892 | CH2 | TRP | A | 91 | 159.585 | 40.876 | 10.338 | 1.00 | 36.22 |
| ATOM | 893 | C | TRP | A | 91 | 157.218 | 38.520 | 14.339 | 1.00 | 43.43 |
| ATOM | 894 | O | TRP | A | 91 | 157.460 | 39.717 | 14.509 | 1.00 | 44.14 |
| ATOM | 895 | N | LYS | A | 92 | 158.136 | 37.575 | 14.468 | 1.00 | 44.32 |
| ATOM | 897 | CA | LYS | A | 92 | 159.517 | 37.892 | 14.789 | 1.00 | 44.34 |
| ATOM | 898 | CB | LYS | A | 92 | 159.717 | 38.076 | 16.302 | 1.00 | 47.57 |
| ATOM | 899 | CG | LYS | A | 92 | 159.252 | 36.933 | 17.200 | 1.00 | 53.21 |
| ATOM | 900 | CD | LYS | A | 92 | 159.361 | 37.354 | 18.678 | 1.00 | 57.24 |
| ATOM | 901 | CE | LYS | A | 92 | 158.844 | 36.283 | 19.650 | 1.00 | 59.23 |
| ATOM | 902 | NZ | LYS | A | 92 | 158.816 | 36.742 | 21.083 | 1.00 | 59.30 |
| ATOM | 906 | C | LYS | A | 92 | 160.409 | 36.801 | 14.227 | 1.00 | 42.18 |
| ATOM | 907 | O | LYS | A | 92 | 160.052 | 35.623 | 14.255 | 1.00 | 43.11 |
| ATOM | 908 | N | ALA | A | 93 | 161.521 | 37.214 | 13.632 | 1.00 | 39.24 |
| ATOM | 910 | CA | ALA | A | 93 | 162.471 | 36.294 | 13.033 | 1.00 | 36.99 |
| ATOM | 911 | CB | ALA | A | 93 | 162.410 | 36.392 | 11.515 | 1.00 | 36.10 |
| ATOM | 912 | C | ALA | A | 93 | 163.850 | 36.675 | 13.525 | 1.00 | 37.81 |
| ATOM | 913 | O | ALA | A | 93 | 164.130 | 37.851 | 13.747 | 1.00 | 39.61 |
| ATOM | 914 | N | GLN | A | 94 | 164.706 | 35.679 | 13.705 | 1.00 | 39.81 |
| ATOM | 916 | CA | GLN | A | 94 | 166.060 | 35.919 | 14.173 | 1.00 | 41.70 |
| ATOM | 917 | CB | GLN | A | 94 | 166.184 | 35.541 | 15.647 | 1.00 | 45.63 |
| ATOM | 918 | CG | GLN | A | 94 | 167.552 | 35.803 | 16.237 | 1.00 | 55.83 |
| ATOM | 919 | CD | GLN | A | 94 | 167.559 | 35.745 | 17.750 | 1.00 | 63.01 |
| ATOM | 920 | OE1 | GLN | A | 94 | 168.328 | 36.456 | 18.406 | 1.00 | 67.41 |
| ATOM | 921 | NE2 | GLN | A | 94 | 166.703 | 34.899 | 18.320 | 1.00 | 66.99 |
| ATOM | 924 | C | GLN | A | 94 | 167.067 | 35.139 | 13.340 | 1.00 | 41.13 |
| ATOM | 925 | O | GLN | A | 94 | 166.952 | 33.921 | 13.194 | 1.00 | 41.48 |
| ATOM | 926 | N | LYS | A | 95 | 168.024 | 35.865 | 12.771 | 1.00 | 41.51 |
| ATOM | 928 | CA | LYS | A | 95 | 169.084 | 35.302 | 11.938 | 1.00 | 40.50 |
| ATOM | 929 | CB | LYS | A | 95 | 169.000 | 35.901 | 10.531 | 1.00 | 40.86 |
| ATOM | 930 | CG | LYS | A | 95 | 170.099 | 35.478 | 9.580 | 1.00 | 44.00 |
| ATOM | 931 | CD | LYS | A | 95 | 169.849 | 36.030 | 8.175 | 1.00 | 46.44 |
| ATOM | 932 | CE | LYS | A | 95 | 169.767 | 37.553 | 8.161 | 1.00 | 48.82 |
| ATOM | 933 | NZ | LYS | A | 95 | 169.529 | 38.117 | 6.799 | 1.00 | 49.76 |
| ATOM | 937 | C | LYS | A | 95 | 170.381 | 35.705 | 12.631 | 1.00 | 39.51 |
| ATOM | 938 | O | LYS | A | 95 | 170.781 | 36.869 | 12.594 | 1.00 | 39.68 |
| ATOM | 939 | N | ARG | A | 96 | 171.000 | 34.743 | 13.304 | 1.00 | 39.70 |
| ATOM | 941 | CA | ARG | A | 96 | 172.229 | 34.965 | 14.065 | 1.00 | 38.81 |
| ATOM | 942 | CB | ARG | A | 96 | 173.386 | 35.432 | 13.159 | 1.00 | 38.58 |
| ATOM | 943 | CG | ARG | A | 96 | 174.757 | 35.415 | 13.848 | 1.00 | 36.99 |
| ATOM | 944 | CD | ARG | A | 96 | 175.919 | 35.584 | 12.869 | 1.00 | 35.28 |
| ATOM | 945 | NE | ARG | A | 96 | 177.207 | 35.640 | 13.562 | 1.00 | 37.59 |
| ATOM | 947 | CZ | ARG | A | 96 | 177.887 | 34.582 | 14.004 | 1.00 | 38.32 |
| ATOM | 948 | NH1 | ARG | A | 96 | 177.425 | 33.351 | 13.832 | 1.00 | 41.87 |
| ATOM | 951 | NH2 | ARG | A | 96 | 179.024 | 34.758 | 14.658 | 1.00 | 34.85 |
| ATOM | 954 | C | ARG | A | 96 | 171.909 | 35.972 | 15.183 | 1.00 | 38.25 |
| ATOM | 955 | O | ARG | A | 96 | 171.091 | 35.672 | 16.048 | 1.00 | 38.45 |
| ATOM | 956 | N | PHE | A | 97 | 172.506 | 37.161 | 15.158 | 1.00 | 36.05 |
| ATOM | 958 | CA | PHE | A | 97 | 172.236 | 38.157 | 16.189 | 1.00 | 32.76 |
| ATOM | 959 | CB | PHE | A | 97 | 173.521 | 38.856 | 16.634 | 1.00 | 29.39 |
| ATOM | 960 | CG | PHE | A | 97 | 174.503 | 37.958 | 17.311 | 1.00 | 27.30 |
| ATOM | 961 | CD1 | PHE | A | 97 | 174.214 | 37.392 | 18.538 | 1.00 | 26.91 |
| ATOM | 962 | CD2 | PHE | A | 97 | 175.733 | 37.695 | 16.728 | 1.00 | 29.62 |
| ATOM | 963 | CE1 | PHE | A | 97 | 175.140 | 36.578 | 19.175 | 1.00 | 28.35 |
| ATOM | 964 | CE2 | PHE | A | 97 | 176.661 | 36.886 | 17.358 | 1.00 | 27.60 |
| ATOM | 965 | CZ | PHE | A | 97 | 176.364 | 36.327 | 18.582 | 1.00 | 28.30 |
| ATOM | 966 | C | PHE | A | 97 | 171.261 | 39.208 | 15.693 | 1.00 | 34.25 |
| ATOM | 967 | O | PHE | A | 97 | 171.016 | 40.199 | 16.387 | 1.00 | 33.16 |
| ATOM | 968 | N | LEU | A | 98 | 170.739 | 39.012 | 14.484 | 1.00 | 36.60 |
| ATOM | 970 | CA | LEU | A | 98 | 169.790 | 39.952 | 13.888 | 1.00 | 39.31 |
| ATOM | 971 | CB | LEU | A | 98 | 169.874 | 39.905 | 12.361 | 1.00 | 38.74 |
| ATOM | 972 | CG | LEU | A | 98 | 169.875 | 41.238 | 11.608 | 1.00 | 38.17 |
| ATOM | 973 | CD1 | LEU | A | 98 | 169.883 | 40.951 | 10.128 | 1.00 | 40.67 |
| ATOM | 974 | CD2 | LEU | A | 98 | 168.680 | 42.093 | 11.971 | 1.00 | 38.92 |
| ATOM | 975 | C | LEU | A | 98 | 168.366 | 39.631 | 14.317 | 1.00 | 40.51 |
| ATOM | 976 | O | LEU | A | 98 | 167.854 | 38.554 | 14.016 | 1.00 | 42.93 |
| ATOM | 977 | N | LYS | A | 99 | 167.737 | 40.570 | 15.018 | 1.00 | 41.98 |
| ATOM | 979 | CA | LYS | A | 99 | 166.365 | 40.410 | 15.488 | 1.00 | 42.37 |
| ATOM | 980 | CB | LYS | A | 99 | 166.258 | 40.705 | 16.989 | 1.00 | 41.90 |
| ATOM | 981 | CG | LYS | A | 99 | 166.690 | 39.566 | 17.898 | 1.00 | 45.06 |
| ATOM | 982 | CD | LYS | A | 99 | 166.389 | 39.882 | 19.361 | 1.00 | 47.68 |
| ATOM | 983 | CE | LYS | A | 99 | 166.638 | 38.671 | 20.250 | 1.00 | 51.09 |
| ATOM | 984 | NZ | LYS | A | 99 | 166.251 | 38.895 | 21.674 | 1.00 | 53.87 |
| ATOM | 988 | C | LYS | A | 99 | 165.440 | 41.351 | 14.727 | 1.00 | 43.86 |
| ATOM | 989 | O | LYS | A | 99 | 165.634 | 42.573 | 14.739 | 1.00 | 45.41 |
| ATOM | 990 | N | MET | A | 100 | 164.438 | 40.785 | 14.064 | 1.00 | 43.74 |
| ATOM | 992 | CA | MET | A | 100 | 163.479 | 41.575 | 13.310 | 1.00 | 43.78 |
| ATOM | 993 | CB | MET | A | 100 | 163.576 | 41.234 | 11.826 | 1.00 | 44.48 |
| ATOM | 994 | CG | MET | A | 100 | 164.885 | 41.651 | 11.195 | 1.00 | 45.32 |
| ATOM | 995 | SD | MET | A | 100 | 165.190 | 40.802 | 9.654 | 1.00 | 49.68 |
| ATOM | 996 | CE | MET | A | 100 | 165.736 | 39.168 | 10.290 | 1.00 | 44.16 |
| ATOM | 997 | C | MET | A | 100 | 162.078 | 41.292 | 13.832 | 1.00 | 44.78 |
| ATOM | 998 | O | MET | A | 100 | 161.693 | 40.132 | 13.987 | 1.00 | 44.97 |
| ATOM | 999 | N | SER | A | 101 | 161.335 | 42.352 | 14.140 | 1.00 | 46.57 |
| ATOM | 1001 | CA | SER | A | 101 | 159.971 | 42.221 | 14.650 | 1.00 | 47.86 |
| ATOM | 1002 | CB | SER | A | 101 | 159.934 | 42.469 | 16.162 | 1.00 | 49.20 |
| ATOM | 1003 | OG | SER | A | 101 | 160.560 | 43.693 | 16.509 | 1.00 | 51.70 |
| ATOM | 1005 | C | SER | A | 101 | 159.009 | 43.168 | 13.937 | 1.00 | 48.68 |
| ATOM | 1006 | O | SER | A | 101 | 159.428 | 44.161 | 13.332 | 1.00 | 48.84 |
| ATOM | 1007 | N | GLY | A | 102 | 157.722 | 42.839 | 13.986 | 1.00 | 49.46 |
| ATOM | 1009 | CA | GLY | A | 102 | 156.716 | 43.664 | 13.343 | 1.00 | 49.50 |
| ATOM | 1010 | C | GLY | A | 102 | 155.359 | 42.989 | 13.341 | 1.00 | 48.98 |
| ATOM | 1011 | O | GLY | A | 102 | 155.251 | 41.795 | 13.613 | 1.00 | 50.09 |
| ATOM | 1012 | N | ASN | A | 103 | 154.317 | 43.766 | 13.079 | 1.00 | 48.68 |
| ATOM | 1014 | CA | ASN | A | 103 | 152.957 | 43.243 | 13.042 | 1.00 | 47.35 |
| ATOM | 1015 | CB | ASN | A | 103 | 151.945 | 44.349 | 13.391 | 1.00 | 49.87 |
| ATOM | 1016 | CG | ASN | A | 103 | 152.061 | 44.837 | 14.840 | 1.00 | 51.69 |
| ATOM | 1017 | OD1 | ASN | A | 103 | 153.067 | 45.423 | 15.234 | 1.00 | 53.03 |
| ATOM | 1018 | ND2 | ASN | A | 103 | 151.014 | 44.621 | 15.625 | 1.00 | 53.17 |
| ATOM | 1021 | C | ASN | A | 103 | 152.703 | 42.743 | 11.625 | 1.00 | 46.11 |
| ATOM | 1022 | O | ASN | A | 103 | 153.459 | 43.073 | 10.708 | 1.00 | 45.99 |
| ATOM | 1023 | N | PHE | A | 104 | 151.667 | 41.926 | 11.451 | 1.00 | 44.40 |
| ATOM | 1025 | CA | PHE | A | 104 | 151.306 | 41.410 | 10.131 | 1.00 | 42.85 |
| ATOM | 1026 | CB | PHE | A | 104 | 151.987 | 40.051 | 9.835 | 1.00 | 42.59 |
| ATOM | 1027 | CG | PHE | A | 104 | 151.319 | 38.849 | 10.482 | 1.00 | 41.53 |
| ATOM | 1028 | CD1 | PHE | A | 104 | 151.650 | 38.458 | 11.776 | 1.00 | 39.25 |
| ATOM | 1029 | CD2 | PHE | A | 104 | 150.380 | 38.095 | 9.778 | 1.00 | 39.79 |
| ATOM | 1030 | CE1 | PHE | A | 104 | 151.058 | 37.339 | 12.357 | 1.00 | 37.60 |
| ATOM | 1031 | CE2 | PHE | A | 104 | 149.784 | 36.976 | 10.353 | 1.00 | 36.72 |
| ATOM | 1032 | CZ | PHE | A | 104 | 150.125 | 36.599 | 11.644 | 1.00 | 37.02 |
| ATOM | 1033 | C | PHE | A | 104 | 149.788 | 41.303 | 10.006 | 1.00 | 42.22 |
| ATOM | 1034 | O | PHE | A | 104 | 149.081 | 41.251 | 11.014 | 1.00 | 43.09 |
| ATOM | 1035 | N | ASP | A | 105 | 149.298 | 41.346 | 8.771 | 1.00 | 41.99 |
| ATOM | 1037 | CA | ASP | A | 105 | 147.874 | 41.227 | 8.475 | 1.00 | 41.67 |
| ATOM | 1038 | CB | ASP | A | 105 | 147.352 | 42.455 | 7.724 | 1.00 | 43.33 |
| ATOM | 1039 | CG | ASP | A | 105 | 146.986 | 43.602 | 8.648 | 1.00 | 46.56 |
| ATOM | 1040 | OD1 | ASP | A | 105 | 146.898 | 43.395 | 9.876 | 1.00 | 49.37 |
| ATOM | 1041 | OD2 | ASP | A | 105 | 146.770 | 44.720 | 8.141 | 1.00 | 51.38 |
| ATOM | 1042 | C | ASP | A | 105 | 147.658 | 39.985 | 7.622 | 1.00 | 41.95 |
| ATOM | 1043 | O | ASP | A | 105 | 148.470 | 39.655 | 6.758 | 1.00 | 42.85 |
| ATOM | 1044 | N | LEU | A | 106 | 146.538 | 39.322 | 7.841 | 1.00 | 40.99 |
| ATOM | 1046 | CA | LEU | A | 106 | 146.220 | 38.115 | 7.115 | 1.00 | 39.85 |
| ATOM | 1047 | CB | LEU | A | 106 | 146.424 | 36.934 | 8.058 | 1.00 | 38.87 |
| ATOM | 1048 | CG | LEU | A | 106 | 146.224 | 35.489 | 7.629 | 1.00 | 39.36 |
| ATOM | 1049 | CD1 | LEU | A | 106 | 146.825 | 34.605 | 8.690 | 1.00 | 40.16 |
| ATOM | 1050 | CD2 | LEU | A | 106 | 144.758 | 35.175 | 7.464 | 1.00 | 41.07 |
| ATOM | 1051 | C | LEU | A | 106 | 144.767 | 38.248 | 6.698 | 1.00 | 40.07 |
| ATOM | 1052 | O | LEU | A | 106 | 143.968 | 38.841 | 7.414 | 1.00 | 41.85 |
| ATOM | 1053 | N | SER | A | 107 | 144.427 | 37.748 | 5.523 | 1.00 | 41.19 |
| ATOM | 1055 | CA | SER | A | 107 | 143.054 | 37.830 | 5.061 | 1.00 | 43.48 |
| ATOM | 1056 | CB | SER | A | 107 | 142.911 | 38.907 | 3.979 | 1.00 | 45.09 |
| ATOM | 1057 | OG | SER | A | 107 | 143.440 | 40.155 | 4.402 | 1.00 | 50.35 |
| ATOM | 1059 | C | SER | A | 107 | 142.633 | 36.484 | 4.502 | 1.00 | 43.83 |
| ATOM | 1060 | O | SER | A | 107 | 143.290 | 35.957 | 3.610 | 1.00 | 44.06 |
| ATOM | 1061 | N | ILE | A | 108 | 141.587 | 35.900 | 5.077 | 1.00 | 45.70 |
| ATOM | 1063 | CA | ILE | A | 108 | 141.056 | 34.617 | 4.615 | 1.00 | 47.58 |
| ATOM | 1064 | CB | ILE | A | 108 | 140.829 | 33.644 | 5.789 | 1.00 | 46.97 |
| ATOM | 1065 | CG2 | ILE | A | 108 | 140.174 | 32.368 | 5.296 | 1.00 | 46.17 |
| ATOM | 1066 | CG1 | ILE | A | 108 | 142.157 | 33.327 | 6.474 | 1.00 | 47.60 |
| ATOM | 1067 | CD1 | ILE | A | 108 | 142.042 | 32.368 | 7.637 | 1.00 | 49.72 |
| ATOM | 1068 | C | ILE | A | 108 | 139.716 | 34.922 | 3.959 | 1.00 | 48.97 |
| ATOM | 1069 | O | ILE | A | 108 | 138.818 | 35.433 | 4.619 | 1.00 | 51.76 |
| ATOM | 1070 | N | GLU | A | 109 | 139.582 | 34.652 | 2.664 | 1.00 | 50.18 |
| ATOM | 1072 | CA | GLU | A | 109 | 138.328 | 34.938 | 1.968 | 1.00 | 52.80 |
| ATOM | 1073 | CB | GLU | A | 109 | 138.506 | 36.067 | 0.956 | 1.00 | 58.29 |
| ATOM | 1074 | CG | GLU | A | 109 | 138.871 | 37.409 | 1.556 | 1.00 | 67.61 |
| ATOM | 1075 | CD | GLU | A | 109 | 138.896 | 38.510 | 0.518 | 1.00 | 72.52 |
| ATOM | 1076 | OE1 | GLU | A | 109 | 139.591 | 38.345 | −0.510 | 1.00 | 76.73 |
| ATOM | 1077 | OE2 | GLU | A | 109 | 138.212 | 39.536 | 0.725 | 1.00 | 74.66 |
| ATOM | 1078 | C | GLU | A | 109 | 137.771 | 33.733 | 1.244 | 1.00 | 51.51 |
| ATOM | 1079 | O | GLU | A | 109 | 138.449 | 32.717 | 1.091 | 1.00 | 51.13 |
| ATOM | 1080 | N | GLY | A | 110 | 136.539 | 33.867 | 0.767 | 1.00 | 50.94 |
| ATOM | 1082 | CA | GLY | A | 110 | 135.904 | 32.778 | 0.051 | 1.00 | 49.27 |
| ATOM | 1083 | C | GLY | A | 110 | 135.714 | 31.551 | 0.920 | 1.00 | 48.08 |
| ATOM | 1084 | O | GLY | A | 110 | 135.972 | 30.419 | 0.488 | 1.00 | 47.03 |
| ATOM | 1085 | N | MET | A | 111 | 135.318 | 31.784 | 2.168 | 1.00 | 46.98 |
| ATOM | 1087 | CA | MET | A | 111 | 135.078 | 30.699 | 3.104 | 1.00 | 44.78 |
| ATOM | 1088 | CB | MET | A | 111 | 135.376 | 31.143 | 4.536 | 1.00 | 44.81 |
| ATOM | 1089 | CG | MET | A | 111 | 135.062 | 30.092 | 5.570 | 1.00 | 44.07 |
| ATOM | 1090 | SD | MET | A | 111 | 135.669 | 30.518 | 7.178 | 1.00 | 49.80 |
| ATOM | 1091 | CE | MET | A | 111 | 136.833 | 29.192 | 7.434 | 1.00 | 46.06 |
| ATOM | 1092 | C | MET | A | 111 | 133.630 | 30.242 | 2.970 | 1.00 | 42.64 |
| ATOM | 1093 | O | MET | A | 111 | 132.712 | 31.061 | 2.960 | 1.00 | 40.20 |
| ATOM | 1094 | N | SER | A | 112 | 133.446 | 28.935 | 2.846 | 1.00 | 41.52 |
| ATOM | 1096 | CA | SER | A | 112 | 132.133 | 28.334 | 2.708 | 1.00 | 41.82 |
| ATOM | 1097 | CB | SER | A | 112 | 132.045 | 27.607 | 1.360 | 1.00 | 43.85 |
| ATOM | 1098 | OG | SER | A | 112 | 130.997 | 26.648 | 1.321 | 1.00 | 49.86 |
| ATOM | 1100 | C | SER | A | 112 | 131.908 | 27.363 | 3.861 | 1.00 | 41.93 |
| ATOM | 1101 | O | SER | A | 112 | 132.725 | 26.467 | 4.097 | 1.00 | 41.84 |
| ATOM | 1102 | N | ILE | A | 113 | 130.816 | 27.569 | 4.592 | 1.00 | 42.07 |
| ATOM | 1104 | CA | ILE | A | 113 | 130.444 | 26.722 | 5.725 | 1.00 | 40.13 |
| ATOM | 1105 | CB | ILE | A | 113 | 130.310 | 27.546 | 7.027 | 1.00 | 39.38 |
| ATOM | 1106 | CG2 | ILE | A | 113 | 130.227 | 26.620 | 8.231 | 1.00 | 39.63 |
| ATOM | 1107 | CG1 | ILE | A | 113 | 131.505 | 28.480 | 7.190 | 1.00 | 35.78 |
| ATOM | 1108 | CD1 | ILE | A | 113 | 131.284 | 29.547 | 8.225 | 1.00 | 38.78 |
| ATOM | 1109 | C | ILE | A | 113 | 129.083 | 26.105 | 5.405 | 1.00 | 40.48 |
| ATOM | 1110 | O | ILE | A | 113 | 128.118 | 26.821 | 5.117 | 1.00 | 40.10 |
| ATOM | 1111 | N | SER | A | 114 | 129.016 | 24.780 | 5.414 | 1.00 | 40.69 |
| ATOM | 1113 | CA | SER | A | 114 | 127.775 | 24.076 | 5.126 | 1.00 | 39.86 |
| ATOM | 1114 | CB | SER | A | 114 | 127.915 | 23.277 | 3.835 | 1.00 | 39.27 |
| ATOM | 1115 | OG | SER | A | 114 | 126.695 | 22.645 | 3.486 | 1.00 | 45.23 |
| ATOM | 1117 | C | SER | A | 114 | 127.489 | 23.152 | 6.297 | 1.00 | 40.43 |
| ATOM | 1118 | O | SER | A | 114 | 128.370 | 22.407 | 6.730 | 1.00 | 42.01 |
| ATOM | 1119 | N | ALA | A | 115 | 126.261 | 23.198 | 6.804 | 1.00 | 40.94 |
| ATOM | 1121 | CA | ALA | A | 115 | 125.863 | 22.380 | 7.947 | 1.00 | 40.28 |
| ATOM | 1122 | CB | ALA | A | 115 | 125.990 | 23.186 | 9.227 | 1.00 | 41.97 |
| ATOM | 1123 | C | ALA | A | 115 | 124.446 | 21.856 | 7.814 | 1.00 | 39.55 |
| ATOM | 1124 | O | ALA | A | 115 | 123.551 | 22.581 | 7.385 | 1.00 | 38.65 |
| ATOM | 1125 | N | ASP | A | 116 | 124.248 | 20.603 | 8.213 | 1.00 | 39.92 |
| ATOM | 1127 | CA | ASP | A | 116 | 122.943 | 19.954 | 8.155 | 1.00 | 40.51 |
| ATOM | 1128 | CB | ASP | A | 116 | 123.092 | 18.513 | 7.672 | 1.00 | 44.07 |
| ATOM | 1129 | CG | ASP | A | 116 | 123.359 | 18.421 | 6.184 | 1.00 | 48.91 |
| ATOM | 1130 | OD1 | ASP | A | 116 | 124.455 | 18.829 | 5.741 | 1.00 | 53.18 |
| ATOM | 1131 | OD2 | ASP | A | 116 | 122.467 | 17.940 | 5.455 | 1.00 | 52.27 |
| ATOM | 1132 | C | ASP | A | 116 | 122.241 | 19.956 | 9.504 | 1.00 | 39.36 |
| ATOM | 1133 | O | ASP | A | 116 | 122.804 | 19.501 | 10.495 | 1.00 | 40.86 |
| ATOM | 1134 | N | LEU | A | 117 | 121.009 | 20.459 | 9.537 | 1.00 | 38.66 |
| ATOM | 1136 | CA | LEU | A | 117 | 120.220 | 20.508 | 10.766 | 1.00 | 35.83 |
| ATOM | 1137 | CB | LEU | A | 117 | 119.578 | 21.888 | 10.944 | 1.00 | 34.68 |
| ATOM | 1138 | CG | LEU | A | 117 | 120.493 | 23.096 | 11.140 | 1.00 | 34.59 |
| ATOM | 1139 | CD1 | LEU | A | 117 | 119.667 | 24.361 | 11.279 | 1.00 | 35.67 |
| ATOM | 1140 | CD2 | LEU | A | 117 | 121.346 | 22.895 | 12.370 | 1.00 | 35.88 |
| ATOM | 1141 | C | LEU | A | 117 | 119.131 | 19.437 | 10.721 | 1.00 | 35.13 |
| ATOM | 1142 | O | LEU | A | 117 | 118.287 | 19.435 | 9.824 | 1.00 | 35.15 |
| ATOM | 1143 | N | LYS | A | 118 | 119.155 | 18.531 | 11.691 | 1.00 | 35.41 |
| ATOM | 1145 | CA | LYS | A | 118 | 118.180 | 17.450 | 11.776 | 1.00 | 34.03 |
| ATOM | 1146 | CB | LYS | A | 118 | 118.896 | 16.128 | 12.047 | 1.00 | 36.61 |
| ATOM | 1147 | CG | LYS | A | 118 | 118.027 | 14.904 | 11.907 | 1.00 | 41.84 |
| ATOM | 1148 | CD | LYS | A | 118 | 118.870 | 13.640 | 11.890 | 1.00 | 45.83 |
| ATOM | 1149 | CE | LYS | A | 118 | 117.998 | 12.400 | 11.773 | 1.00 | 49.70 |
| ATOM | 1150 | NZ | LYS | A | 118 | 117.098 | 12.434 | 10.576 | 1.00 | 52.85 |
| ATOM | 1154 | C | LYS | A | 118 | 117.184 | 17.760 | 12.884 | 1.00 | 32.08 |
| ATOM | 1155 | O | LYS | A | 118 | 117.572 | 18.045 | 14.014 | 1.00 | 30.72 |
| ATOM | 1156 | N | LEU | A | 119 | 115.900 | 17.737 | 12.545 | 1.00 | 34.05 |
| ATOM | 1158 | CA | LEU | A | 119 | 114.838 | 18.036 | 13.503 | 1.00 | 33.03 |
| ATOM | 1159 | CB | LEU | A | 119 | 113.782 | 18.917 | 12.844 | 1.00 | 30.64 |
| ATOM | 1160 | CG | LEU | A | 119 | 114.277 | 20.278 | 12.372 | 1.00 | 26.32 |
| ATOM | 1161 | CD1 | LEU | A | 119 | 113.434 | 20.751 | 11.229 | 1.00 | 27.43 |
| ATOM | 1162 | CD2 | LEU | A | 119 | 114.230 | 21.258 | 13.511 | 1.00 | 29.44 |
| ATOM | 1163 | C | LEU | A | 119 | 114.192 | 16.771 | 14.065 | 1.00 | 33.62 |
| ATOM | 1164 | O | LEU | A | 119 | 113.904 | 15.819 | 13.334 | 1.00 | 32.69 |
| ATOM | 1165 | N | GLY | A | 120 | 113.952 | 16.776 | 15.368 | 1.00 | 33.78 |
| ATOM | 1167 | CA | GLY | A | 120 | 113.353 | 15.624 | 16.000 | 1.00 | 33.21 |
| ATOM | 1168 | C | GLY | A | 120 | 112.431 | 16.059 | 17.109 | 1.00 | 34.60 |
| ATOM | 1169 | O | GLY | A | 120 | 112.238 | 17.250 | 17.340 | 1.00 | 34.75 |
| ATOM | 1170 | N | SER | A | 121 | 111.883 | 15.086 | 17.817 | 1.00 | 37.06 |
| ATOM | 1172 | CA | SER | A | 121 | 110.961 | 15.355 | 18.902 | 1.00 | 40.83 |
| ATOM | 1173 | CB | SER | A | 121 | 109.541 | 14.982 | 18.464 | 1.00 | 41.68 |
| ATOM | 1174 | OG | SER | A | 121 | 108.638 | 14.924 | 19.554 | 1.00 | 44.95 |
| ATOM | 1176 | C | SER | A | 121 | 111.357 | 14.527 | 20.108 | 1.00 | 43.32 |
| ATOM | 1177 | O | SER | A | 121 | 111.990 | 13.476 | 19.975 | 1.00 | 44.77 |
| ATOM | 1178 | N | ASN | A | 122 | 111.034 | 15.038 | 21.287 | 1.00 | 45.49 |
| ATOM | 1180 | CA | ASN | A | 122 | 111.308 | 14.330 | 22.525 | 1.00 | 48.51 |
| ATOM | 1181 | CB | ASN | A | 122 | 112.014 | 15.244 | 23.523 | 1.00 | 47.83 |
| ATOM | 1182 | CG | ASN | A | 122 | 112.693 | 14.474 | 24.633 | 1.00 | 49.31 |
| ATOM | 1183 | OD1 | ASN | A | 122 | 112.445 | 13.283 | 24.827 | 1.00 | 48.55 |
| ATOM | 1184 | ND2 | ASN | A | 122 | 113.574 | 15.146 | 25.359 | 1.00 | 52.06 |
| ATOM | 1187 | C | ASN | A | 122 | 109.925 | 13.955 | 23.040 | 1.00 | 50.18 |
| ATOM | 1188 | O | ASN | A | 122 | 109.197 | 14.810 | 23.534 | 1.00 | 51.08 |
| ATOM | 1189 | N | PRO | A | 123 | 109.530 | 12.678 | 22.895 | 1.00 | 51.66 |
| ATOM | 1190 | CD | PRO | A | 123 | 110.378 | 11.593 | 22.375 | 1.00 | 51.97 |
| ATOM | 1191 | CA | PRO | A | 123 | 108.229 | 12.152 | 23.323 | 1.00 | 51.81 |
| ATOM | 1192 | CB | PRO | A | 123 | 108.291 | 10.696 | 22.875 | 1.00 | 52.33 |
| ATOM | 1193 | CG | PRO | A | 123 | 109.744 | 10.377 | 22.996 | 1.00 | 52.53 |
| ATOM | 1194 | C | PRO | A | 123 | 107.903 | 12.255 | 24.808 | 1.00 | 52.83 |
| ATOM | 1195 | O | PRO | A | 123 | 106.777 | 12.593 | 25.171 | 1.00 | 54.24 |
| ATOM | 1196 | N | THR | A | 124 | 108.879 | 11.969 | 25.664 | 1.00 | 53.70 |
| ATOM | 1198 | CA | THR | A | 124 | 108.658 | 12.016 | 27.109 | 1.00 | 55.10 |
| ATOM | 1199 | CB | THR | A | 124 | 109.782 | 11.285 | 27.873 | 1.00 | 55.63 |
| ATOM | 1200 | OG1 | THR | A | 124 | 111.038 | 11.556 | 27.244 | 1.00 | 57.26 |
| ATOM | 1202 | CG2 | THR | A | 124 | 109.536 | 9.782 | 27.879 | 1.00 | 57.40 |
| ATOM | 1203 | C | THR | A | 124 | 108.467 | 13.418 | 27.687 | 1.00 | 54.39 |
| ATOM | 1204 | O | THR | A | 124 | 108.031 | 13.568 | 28.833 | 1.00 | 56.00 |
| ATOM | 1205 | N | SER | A | 125 | 108.794 | 14.439 | 26.901 | 1.00 | 52.41 |
| ATOM | 1207 | CA | SER | A | 125 | 108.647 | 15.824 | 27.345 | 1.00 | 49.57 |
| ATOM | 1208 | CB | SER | A | 125 | 110.023 | 16.448 | 27.598 | 1.00 | 49.34 |
| ATOM | 1209 | OG | SER | A | 125 | 110.859 | 16.328 | 26.459 | 1.00 | 50.88 |
| ATOM | 1211 | C | SER | A | 125 | 107.855 | 16.676 | 26.355 | 1.00 | 46.68 |
| ATOM | 1212 | O | SER | A | 125 | 107.366 | 17.747 | 26.702 | 1.00 | 46.69 |
| ATOM | 1213 | N | GLY | A | 126 | 107.734 | 16.191 | 25.123 | 1.00 | 44.84 |
| ATOM | 1215 | CA | GLY | A | 126 | 107.008 | 16.906 | 24.091 | 1.00 | 42.00 |
| ATOM | 1216 | C | GLY | A | 126 | 107.739 | 18.113 | 23.537 | 1.00 | 40.90 |
| ATOM | 1217 | O | GLY | A | 126 | 107.125 | 18.953 | 22.885 | 1.00 | 41.68 |
| ATOM | 1218 | N | LYS | A | 127 | 109.044 | 18.201 | 23.780 | 1.00 | 40.27 |
| ATOM | 1220 | CA | LYS | A | 127 | 109.846 | 19.330 | 23.303 | 1.00 | 38.06 |
| ATOM | 1221 | CB | LYS | A | 127 | 110.914 | 19.708 | 24.340 | 1.00 | 41.48 |
| ATOM | 1222 | CG | LYS | A | 127 | 110.408 | 20.194 | 25.705 | 1.00 | 44.59 |
| ATOM | 1223 | CD | LYS | A | 127 | 109.966 | 21.657 | 25.697 | 1.00 | 48.40 |
| ATOM | 1224 | CE | LYS | A | 127 | 109.668 | 22.142 | 27.118 | 1.00 | 49.68 |
| ATOM | 1225 | NZ | LYS | A | 127 | 109.045 | 23.498 | 27.151 | 1.00 | 50.76 |
| ATOM | 1229 | C | LYS | A | 127 | 110.533 | 18.984 | 21.980 | 1.00 | 36.38 |
| ATOM | 1230 | O | LYS | A | 127 | 110.916 | 17.827 | 21.751 | 1.00 | 36.93 |
| ATOM | 1231 | N | PRO | A | 128 | 110.705 | 19.980 | 21.093 | 1.00 | 33.49 |
| ATOM | 1232 | CD | PRO | A | 128 | 110.310 | 21.395 | 21.226 | 1.00 | 31.99 |
| ATOM | 1233 | CA | PRO | A | 128 | 111.354 | 19.747 | 19.803 | 1.00 | 31.41 |
| ATOM | 1234 | CB | PRO | A | 128 | 111.023 | 21.020 | 19.037 | 1.00 | 31.27 |
| ATOM | 1235 | CG | PRO | A | 128 | 111.085 | 22.056 | 20.109 | 1.00 | 29.23 |
| ATOM | 1236 | C | PRO | A | 128 | 112.853 | 19.611 | 20.007 | 1.00 | 31.58 |
| ATOM | 1237 | O | PRO | A | 128 | 113.389 | 20.080 | 21.010 | 1.00 | 31.45 |
| ATOM | 1238 | N | THR | A | 129 | 113.523 | 18.936 | 19.087 | 1.00 | 31.21 |
| ATOM | 1240 | CA | THR | A | 129 | 114.960 | 18.779 | 19.181 | 1.00 | 30.79 |
| ATOM | 1241 | CB | THR | A | 129 | 115.377 | 17.332 | 19.510 | 1.00 | 29.38 |
| ATOM | 1242 | OG1 | THR | A | 129 | 114.952 | 16.451 | 18.467 | 1.00 | 30.58 |
| ATOM | 1244 | CG2 | THR | A | 129 | 114.773 | 16.886 | 20.818 | 1.00 | 27.50 |
| ATOM | 1245 | C | THR | A | 129 | 115.568 | 19.185 | 17.856 | 1.00 | 32.76 |
| ATOM | 1246 | O | THR | A | 129 | 114.907 | 19.148 | 16.813 | 1.00 | 32.88 |
| ATOM | 1247 | N | ILE | A | 130 | 116.828 | 19.582 | 17.904 | 1.00 | 35.82 |
| ATOM | 1249 | CA | ILE | A | 130 | 117.554 | 19.999 | 16.717 | 1.00 | 37.64 |
| ATOM | 1250 | CB | ILE | A | 130 | 117.302 | 21.506 | 16.401 | 1.00 | 38.78 |
| ATOM | 1251 | CG2 | ILE | A | 130 | 117.277 | 22.335 | 17.665 | 1.00 | 40.02 |
| ATOM | 1252 | CG1 | ILE | A | 130 | 118.345 | 22.042 | 15.425 | 1.00 | 38.94 |
| ATOM | 1253 | CD1 | ILE | A | 130 | 118.138 | 21.591 | 14.014 | 1.00 | 42.34 |
| ATOM | 1254 | C | ILE | A | 130 | 119.026 | 19.740 | 16.985 | 1.00 | 38.69 |
| ATOM | 1255 | O | ILE | A | 130 | 119.534 | 20.085 | 18.048 | 1.00 | 40.22 |
| ATOM | 1256 | N | THR | A | 131 | 119.681 | 19.039 | 16.069 | 1.00 | 39.62 |
| ATOM | 1258 | CA | THR | A | 131 | 121.098 | 18.748 | 16.217 | 1.00 | 40.39 |
| ATOM | 1259 | CB | THR | A | 131 | 121.351 | 17.318 | 16.747 | 1.00 | 40.39 |
| ATOM | 1260 | OG1 | THR | A | 131 | 120.833 | 16.354 | 15.825 | 1.00 | 40.07 |
| ATOM | 1262 | CG2 | THR | A | 131 | 120.696 | 17.124 | 18.113 | 1.00 | 43.21 |
| ATOM | 1263 | C | THR | A | 131 | 121.788 | 18.927 | 14.878 | 1.00 | 41.17 |
| ATOM | 1264 | O | THR | A | 131 | 121.139 | 18.951 | 13.839 | 1.00 | 41.86 |
| ATOM | 1265 | N | CYS | A | 132 | 123.099 | 19.107 | 14.911 | 1.00 | 42.72 |
| ATOM | 1267 | CA | CYS | A | 132 | 123.875 | 19.284 | 13.696 | 1.00 | 42.81 |
| ATOM | 1268 | CB | CYS | A | 132 | 124.963 | 20.326 | 13.932 | 1.00 | 42.56 |
| ATOM | 1269 | SG | CYS | A | 132 | 126.009 | 20.629 | 12.530 | 1.00 | 38.05 |
| ATOM | 1270 | C | CYS | A | 132 | 124.494 | 17.940 | 13.333 | 1.00 | 44.48 |
| ATOM | 1271 | O | CYS | A | 132 | 125.370 | 17.446 | 14.041 | 1.00 | 46.08 |
| ATOM | 1272 | N | SER | A | 133 | 124.019 | 17.344 | 12.244 | 1.00 | 45.05 |
| ATOM | 1274 | CA | SER | A | 133 | 124.508 | 16.045 | 11.794 | 1.00 | 45.93 |
| ATOM | 1275 | CB | SER | A | 133 | 123.410 | 15.315 | 11.015 | 1.00 | 47.62 |
| ATOM | 1276 | OG | SER | A | 133 | 122.962 | 16.082 | 9.908 | 1.00 | 51.90 |
| ATOM | 1278 | C | SER | A | 133 | 125.793 | 16.072 | 10.966 | 1.00 | 45.39 |
| ATOM | 1279 | O | SER | A | 133 | 126.512 | 15.078 | 10.916 | 1.00 | 46.00 |
| ATOM | 1280 | N | SER | A | 134 | 126.056 | 17.182 | 10.282 | 1.00 | 45.72 |
| ATOM | 1282 | CA | SER | A | 134 | 127.257 | 17.322 | 9.453 | 1.00 | 45.69 |
| ATOM | 1283 | CB | SER | A | 134 | 127.048 | 16.672 | 8.073 | 1.00 | 47.17 |
| ATOM | 1284 | OG | SER | A | 134 | 127.221 | 15.263 | 8.110 | 1.00 | 50.20 |
| ATOM | 1286 | C | SER | A | 134 | 127.642 | 18.788 | 9.265 | 1.00 | 44.02 |
| ATOM | 1287 | O | SER | A | 134 | 126.775 | 19.663 | 9.212 | 1.00 | 45.04 |
| ATOM | 1288 | N | CYS | A | 135 | 128.939 | 19.047 | 9.145 | 1.00 | 42.35 |
| ATOM | 1290 | CA | CYS | A | 135 | 129.442 | 20.398 | 8.941 | 1.00 | 40.67 |
| ATOM | 1291 | C | CYS | A | 135 | 130.728 | 20.345 | 8.144 | 1.00 | 41.67 |
| ATOM | 1292 | O | CYS | A | 135 | 131.469 | 19.361 | 8.216 | 1.00 | 40.71 |
| ATOM | 1293 | CB | CYS | A | 135 | 129.718 | 21.084 | 10.274 | 1.00 | 39.61 |
| ATOM | 1294 | SG | CYS | A | 135 | 130.296 | 22.800 | 10.094 | 1.00 | 34.66 |
| ATOM | 1295 | N | SER | A | 136 | 130.975 | 21.383 | 7.354 | 1.00 | 43.65 |
| ATOM | 1297 | CA | SER | A | 136 | 132.192 | 21.451 | 6.562 | 1.00 | 45.71 |
| ATOM | 1298 | CB | SER | A | 136 | 132.131 | 20.504 | 5.357 | 1.00 | 48.55 |
| ATOM | 1299 | OG | SER | A | 136 | 131.231 | 20.961 | 4.363 | 1.00 | 54.17 |
| ATOM | 1301 | C | SER | A | 136 | 132.465 | 22.874 | 6.114 | 1.00 | 45.23 |
| ATOM | 1302 | O | SER | A | 136 | 131.582 | 23.551 | 5.583 | 1.00 | 44.66 |
| ATOM | 1303 | N | SER | A | 137 | 133.673 | 23.339 | 6.411 | 1.00 | 45.29 |
| ATOM | 1305 | CA | SER | A | 137 | 134.110 | 24.671 | 6.040 | 1.00 | 45.90 |
| ATOM | 1306 | CB | SER | A | 137 | 134.896 | 25.302 | 7.186 | 1.00 | 45.32 |
| ATOM | 1307 | OG | SER | A | 137 | 135.302 | 26.620 | 6.867 | 1.00 | 46.54 |
| ATOM | 1309 | C | SER | A | 137 | 134.990 | 24.514 | 4.806 | 1.00 | 47.49 |
| ATOM | 1310 | O | SER | A | 137 | 135.330 | 23.392 | 4.416 | 1.00 | 48.14 |
| ATOM | 1311 | N | HIS | A | 138 | 135.371 | 25.630 | 4.200 | 1.00 | 48.87 |
| ATOM | 1313 | CA | HIS | A | 138 | 136.196 | 25.599 | 3.003 | 1.00 | 49.64 |
| ATOM | 1314 | CB | HIS | A | 138 | 135.338 | 25.149 | 1.811 | 1.00 | 53.41 |
| ATOM | 1315 | CG | HIS | A | 138 | 136.097 | 24.992 | 0.529 | 1.00 | 56.80 |
| ATOM | 1316 | CD2 | HIS | A | 138 | 136.712 | 23.915 | −0.016 | 1.00 | 58.14 |
| ATOM | 1317 | ND1 | HIS | A | 138 | 136.268 | 26.025 | −0.368 | 1.00 | 59.15 |
| ATOM | 1319 | CE1 | HIS | A | 138 | 136.956 | 25.592 | −1.412 | 1.00 | 59.11 |
| ATOM | 1320 | NE2 | HIS | A | 138 | 137.238 | 24.316 | −1.222 | 1.00 | 59.30 |
| ATOM | 1322 | C | HIS | A | 138 | 136.779 | 26.983 | 2.742 | 1.00 | 49.79 |
| ATOM | 1323 | O | HIS | A | 138 | 136.042 | 27.938 | 2.510 | 1.00 | 49.66 |
| ATOM | 1324 | N | ILE | A | 139 | 138.102 | 27.082 | 2.808 | 1.00 | 50.32 |
| ATOM | 1326 | CA | ILE | A | 139 | 138.822 | 28.330 | 2.565 | 1.00 | 50.43 |
| ATOM | 1327 | CB | ILE | A | 139 | 139.937 | 28.519 | 3.606 | 1.00 | 50.04 |
| ATOM | 1328 | CG2 | ILE | A | 139 | 140.876 | 29.651 | 3.195 | 1.00 | 46.81 |
| ATOM | 1329 | CG1 | ILE | A | 139 | 139.313 | 28.751 | 4.982 | 1.00 | 49.41 |
| ATOM | 1330 | CD1 | ILE | A | 139 | 140.310 | 28.816 | 6.105 | 1.00 | 53.48 |
| ATOM | 1331 | C | ILE | A | 139 | 139.430 | 28.312 | 1.159 | 1.00 | 51.98 |
| ATOM | 1332 | O | ILE | A | 139 | 140.019 | 27.311 | 0.743 | 1.00 | 51.75 |
| ATOM | 1333 | N | ASN | A | 140 | 139.267 | 29.409 | 0.425 | 1.00 | 53.16 |
| ATOM | 1335 | CA | ASN | A | 140 | 139.792 | 29.503 | −0.935 | 1.00 | 54.65 |
| ATOM | 1336 | CB | ASN | A | 140 | 138.824 | 30.291 | −1.832 | 1.00 | 57.65 |
| ATOM | 1337 | CG | ASN | A | 140 | 139.311 | 30.400 | −3.276 | 1.00 | 60.64 |
| ATOM | 1338 | OD1 | ASN | A | 140 | 139.295 | 31.483 | −3.867 | 1.00 | 61.18 |
| ATOM | 1339 | ND2 | ASN | A | 140 | 139.742 | 29.279 | −3.848 | 1.00 | 62.27 |
| ATOM | 1342 | C | ASN | A | 140 | 141.203 | 30.092 | −1.024 | 1.00 | 53.67 |
| ATOM | 1343 | O | ASN | A | 140 | 142.097 | 29.463 | −1.589 | 1.00 | 53.36 |
| ATOM | 1344 | N | SER | A | 141 | 141.401 | 31.289 | −0.474 | 1.00 | 52.97 |
| ATOM | 1346 | CA | SER | A | 141 | 142.711 | 31.943 | −0.518 | 1.00 | 52.86 |
| ATOM | 1347 | CB | SER | A | 141 | 142.728 | 33.051 | −1.585 | 1.00 | 52.81 |
| ATOM | 1348 | OG | SER | A | 141 | 141.928 | 34.167 | −1.218 | 1.00 | 50.44 |
| ATOM | 1350 | C | SER | A | 141 | 143.114 | 32.526 | 0.832 | 1.00 | 52.49 |
| ATOM | 1351 | O | SER | A | 141 | 142.263 | 32.778 | 1.684 | 1.00 | 54.02 |
| atom | 1352 | N | VAL | A | 142 | 144.415 | 32.724 | 1.023 | 1.00 | 52.25 |
| ATOM | 1354 | CA | VAL | A | 142 | 144.947 | 33.290 | 2.256 | 1.00 | 50.52 |
| ATOM | 1355 | CB | VAL | A | 142 | 145.583 | 32.203 | 3.152 | 1.00 | 49.93 |
| ATOM | 1356 | CG1 | VAL | A | 142 | 146.214 | 32.826 | 4.380 | 1.00 | 49.68 |
| ATOM | 1357 | CG2 | VAL | A | 142 | 144.533 | 31.197 | 3.577 | 1.00 | 50.61 |
| ATOM | 1358 | C | VAL | A | 142 | 145.990 | 34.335 | 1.880 | 1.00 | 51.54 |
| ATOM | 1359 | O | VAL | A | 142 | 147.083 | 34.000 | 1.418 | 1.00 | 50.89 |
| ATOM | 1360 | N | HIS | A | 143 | 145.611 | 35.603 | 2.021 | 1.00 | 54.44 |
| ATOM | 1362 | CA | HIS | A | 143 | 146.470 | 36.742 | 1.709 | 1.00 | 57.63 |
| ATOM | 1363 | CB | HIS | A | 143 | 145.639 | 37.904 | 1.154 | 1.00 | 60.05 |
| ATOM | 1364 | CG | HIS | A | 143 | 145.323 | 37.789 | −0.306 | 1.00 | 65.61 |
| ATOM | 1365 | CD2 | HIS | A | 143 | 145.600 | 38.615 | −1.343 | 1.00 | 67.19 |
| ATOM | 1366 | ND1 | HIS | A | 143 | 144.632 | 36.722 | −0.841 | 1.00 | 67.65 |
| ATOM | 1368 | CE1 | HIS | A | 143 | 144.498 | 36.896 | −2.145 | 1.00 | 68.08 |
| ATOM | 1369 | NE2 | HIS | A | 143 | 145.078 | 38.037 | −2.473 | 1.00 | 67.35 |
| ATOM | 1371 | C | HIS | A | 143 | 147.229 | 37.232 | 2.936 | 1.00 | 58.93 |
| ATOM | 1372 | O | HIS | A | 143 | 146.663 | 37.914 | 3.791 | 1.00 | 58.93 |
| ATOM | 1373 | N | VAL | A | 144 | 148.510 | 36.893 | 3.012 | 1.00 | 60.40 |
| ATOM | 1375 | CA | VAL | A | 144 | 149.347 | 37.316 | 4.124 | 1.00 | 62.35 |
| ATOM | 1376 | CB | VAL | A | 144 | 150.423 | 36.270 | 4.441 | 1.00 | 61.34 |
| ATOM | 1377 | CG1 | VAL | A | 144 | 151.245 | 36.703 | 5.641 | 1.00 | 62.08 |
| ATOM | 1378 | CG2 | VAL | A | 144 | 149.771 | 34.938 | 4.709 | 1.00 | 62.01 |
| ATOM | 1379 | C | VAL | A | 144 | 150.000 | 38.632 | 3.724 | 1.00 | 64.53 |
| ATOM | 1380 | O | VAL | A | 144 | 150.971 | 38.658 | 2.971 | 1.00 | 64.35 |
| ATOM | 1381 | N | HIS | A | 145 | 149.440 | 39.729 | 4.214 | 1.00 | 67.48 |
| ATOM | 1383 | CA | HIS | A | 145 | 149.945 | 41.050 | 3.886 | 1.00 | 71.08 |
| ATOM | 1384 | CB | HIS | A | 145 | 148.791 | 42.045 | 3.771 | 1.00 | 72.13 |
| ATOM | 1385 | CG | HIS | A | 145 | 147.860 | 41.751 | 2.642 | 1.00 | 74.06 |
| ATOM | 1386 | CD2 | HIS | A | 145 | 148.098 | 41.536 | 1.327 | 1.00 | 74.26 |
| ATOM | 1387 | ND1 | HIS | A | 145 | 146.497 | 41.640 | 2.810 | 1.00 | 75.07 |
| ATOM | 1389 | CE1 | HIS | A | 145 | 145.934 | 41.370 | 1.646 | 1.00 | 75.64 |
| ATOM | 1390 | NE2 | HIS | A | 145 | 146.884 | 41.303 | 0.730 | 1.00 | 75.46 |
| ATOM | 1392 | C | HIS | A | 145 | 150.998 | 41.600 | 4.830 | 1.00 | 73.50 |
| ATOM | 1393 | O | HIS | A | 145 | 150.742 | 42.560 | 5.553 | 1.00 | 74.03 |
| ATOM | 1394 | N | ILE | A | 146 | 152.184 | 40.999 | 4.826 | 1.00 | 76.22 |
| ATOM | 1396 | CA | ILE | A | 146 | 153.277 | 41.497 | 5.660 | 1.00 | 78.65 |
| ATOM | 1397 | CB | ILE | A | 146 | 154.282 | 40.374 | 6.055 | 1.00 | 77.20 |
| ATOM | 1398 | CG2 | ILE | A | 146 | 155.320 | 40.905 | 7.037 | 1.00 | 75.80 |
| ATOM | 1399 | CG1 | ILE | A | 146 | 153.545 | 39.214 | 6.729 | 1.00 | 76.58 |
| ATOM | 1400 | CD1 | ILE | A | 146 | 154.451 | 38.091 | 7.184 | 1.00 | 75.69 |
| ATOM | 1401 | C | ILE | A | 146 | 153.972 | 42.574 | 4.815 | 1.00 | 81.85 |
| ATOM | 1402 | O | ILE | A | 146 | 155.107 | 42.970 | 5.082 | 1.00 | 81.33 |
| ATOM | 1403 | N | SER | A | 147 | 153.273 | 43.023 | 3.774 | 1.00 | 85.84 |
| ATOM | 1405 | CA | SER | A | 147 | 153.758 | 44.049 | 2.864 | 1.00 | 89.73 |
| ATOM | 1406 | CB | SER | A | 147 | 152.736 | 44.267 | 1.737 | 1.00 | 89.11 |
| ATOM | 1407 | OG | SER | A | 147 | 152.396 | 43.046 | 1.097 | 1.00 | 89.21 |
| ATOM | 1409 | C | SER | A | 147 | 153.983 | 45.354 | 3.637 | 1.00 | 92.25 |
| ATOM | 1410 | O | SER | A | 147 | 153.080 | 46.188 | 3.753 | 1.00 | 94.01 |
| ATOM | 1411 | N | ALA | A | 148 | 155.182 | 45.494 | 4.197 | 1.00 | 92.99 |
| ATOM | 1413 | CA | ALA | A | 148 | 155.568 | 46.675 | 4.967 | 1.00 | 92.04 |
| ATOM | 1414 | CB | ALA | A | 148 | 155.027 | 46.577 | 6.395 | 1.00 | 92.57 |
| ATOM | 1415 | C | ALA | A | 148 | 157.092 | 46.778 | 4.982 | 1.00 | 91.03 |
| ATOM | 1416 | O | ALA | A | 148 | 157.657 | 47.869 | 5.086 | 1.00 | 92.24 |
| ATOM | 1417 | N | ALA | A | 149 | 157.749 | 45.628 | 4.875 | 1.00 | 88.25 |
| ATOM | 1419 | CA | ALA | A | 149 | 159.201 | 45.558 | 4.864 | 1.00 | 85.00 |
| ATOM | 1420 | CB | ALA | A | 149 | 159.720 | 45.237 | 6.263 | 1.00 | 85.37 |
| ATOM | 1421 | C | ALA | A | 149 | 159.624 | 44.474 | 3.874 | 1.00 | 82.20 |
| ATOM | 1422 | O | ALA | A | 149 | 158.888 | 43.508 | 3.650 | 1.00 | 83.34 |
| ATOM | 1423 | N | SER | A | 150 | 160.785 | 44.657 | 3.252 | 1.00 | 77.07 |
| ATOM | 1425 | CA | SER | A | 150 | 161.304 | 43.690 | 2.293 | 1.00 | 71.37 |
| ATOM | 1426 | CB | SER | A | 150 | 162.463 | 44.313 | 1.509 | 1.00 | 72.17 |
| ATOM | 1427 | OG | SER | A | 150 | 162.092 | 45.574 | 0.968 | 1.00 | 72.35 |
| ATOM | 1429 | C | SER | A | 150 | 161.768 | 42.434 | 3.037 | 1.00 | 66.95 |
| ATOM | 1430 | O | SER | A | 150 | 162.927 | 42.330 | 3.440 | 1.00 | 66.96 |
| ATOM | 1431 | N | VAL | A | 151 | 160.847 | 41.496 | 3.240 | 1.00 | 60.88 |
| ATOM | 1433 | CA | VAL | A | 151 | 161.146 | 40.257 | 3.949 | 1.00 | 54.82 |
| ATOM | 1434 | CB | VAL | A | 151 | 160.736 | 40.381 | 5.446 | 1.00 | 55.79 |
| ATOM | 1435 | CG1 | VAL | A | 151 | 159.233 | 40.593 | 5.584 | 1.00 | 55.13 |
| ATOM | 1436 | CG2 | VAL | A | 151 | 161.206 | 39.176 | 6.242 | 1.00 | 55.16 |
| ATOM | 1437 | C | VAL | A | 151 | 160.417 | 39.104 | 3.259 | 1.00 | 51.49 |
| ATOM | 1438 | O | VAL | A | 151 | 159.721 | 38.308 | 3.887 | 1.00 | 51.42 |
| ATOM | 1439 | N | GLY | A | 152 | 160.639 | 38.992 | 1.957 | 1.00 | 48.87 |
| ATOM | 1441 | CA | GLY | A | 152 | 159.994 | 37.961 | 1.167 | 1.00 | 45.39 |
| ATOM | 1442 | C | GLY | A | 152 | 160.100 | 36.531 | 1.656 | 1.00 | 42.87 |
| ATOM | 1443 | O | GLY | A | 152 | 159.144 | 35.766 | 1.543 | 1.00 | 43.01 |
| ATOM | 1444 | N | TRP | A | 153 | 161.245 | 36.160 | 2.211 | 1.00 | 41.58 |
| ATOM | 1446 | CA | TRP | A | 153 | 161.432 | 34.795 | 2.682 | 1.00 | 41.92 |
| ATOM | 1447 | CB | TRP | A | 153 | 162.853 | 34.591 | 3.196 | 1.00 | 42.78 |
| ATOM | 1448 | CG | TRP | A | 153 | 163.153 | 35.267 | 4.501 | 1.00 | 43.10 |
| ATOM | 1449 | CD2 | TRP | A | 153 | 163.738 | 36.559 | 4.678 | 1.00 | 43.00 |
| ATOM | 1450 | CE2 | TRP | A | 153 | 163.953 | 36.738 | 6.064 | 1.00 | 43.32 |
| ATOM | 1451 | CE3 | TRP | A | 153 | 164.112 | 37.583 | 3.796 | 1.00 | 45.47 |
| ATOM | 1452 | CD1 | TRP | A | 153 | 163.022 | 34.730 | 5.760 | 1.00 | 42.27 |
| ATOM | 1453 | NE1 | TRP | A | 153 | 163.508 | 35.607 | 6.700 | 1.00 | 42.43 |
| ATOM | 1455 | CZ2 | TRP | A | 153 | 164.528 | 37.897 | 6.587 | 1.00 | 44.70 |
| ATOM | 1456 | CZ3 | TRP | A | 153 | 164.686 | 38.738 | 4.317 | 1.00 | 47.45 |
| ATOM | 1457 | CH2 | TRP | A | 153 | 164.889 | 38.885 | 5.701 | 1.00 | 47.30 |
| ATOM | 1458 | C | TRP | A | 153 | 160.442 | 34.393 | 3.763 | 1.00 | 41.11 |
| ATOM | 1459 | O | TRP | A | 153 | 160.053 | 33.236 | 3.852 | 1.00 | 41.06 |
| ATOM | 1460 | N | LEU | A | 154 | 160.045 | 35.357 | 4.585 | 1.00 | 40.39 |
| ATOM | 1462 | CA | LEU | A | 154 | 159.109 | 35.103 | 5.669 | 1.00 | 38.88 |
| ATOM | 1463 | CB | LEU | A | 154 | 159.080 | 36.284 | 6.618 | 1.00 | 37.14 |
| ATOM | 1464 | CG | LEU | A | 154 | 158.607 | 35.931 | 8.011 | 1.00 | 35.27 |
| ATOM | 1465 | CD1 | LEU | A | 154 | 159.491 | 34.860 | 8.602 | 1.00 | 35.45 |
| ATOM | 1466 | CD2 | LEU | A | 154 | 158.655 | 37.166 | 8.838 | 1.00 | 37.10 |
| ATOM | 1467 | C | LEU | A | 154 | 157.721 | 34.847 | 5.115 | 1.00 | 38.87 |
| ATOM | 1468 | O | LEU | A | 154 | 156.997 | 33.985 | 5.611 | 1.00 | 39.93 |
| ATOM | 1469 | N | ILE | A | 155 | 157.357 | 35.596 | 4.079 | 1.00 | 40.01 |
| ATOM | 1471 | CA | ILE | A | 155 | 156.062 | 35.435 | 3.430 | 1.00 | 40.45 |
| ATOM | 1472 | CB | ILE | A | 155 | 155.765 | 36.579 | 2.454 | 1.00 | 39.45 |
| ATOM | 1473 | CG2 | ILE | A | 155 | 154.467 | 36.307 | 1.721 | 1.00 | 39.70 |
| ATOM | 1474 | CG1 | ILE | A | 155 | 155.679 | 37.906 | 3.215 | 1.00 | 39.53 |
| ATOM | 1475 | CD1 | ILE | A | 155 | 155.426 | 39.114 | 2.337 | 1.00 | 39.05 |
| ATOM | 1476 | C | ILE | A | 155 | 156.048 | 34.106 | 2.686 | 1.00 | 42.61 |
| ATOM | 1477 | O | ILE | A | 155 | 155.015 | 33.435 | 2.619 | 1.00 | 44.53 |
| ATOM | 1478 | N | GLN | A | 156 | 157.187 | 33.745 | 2.100 | 1.00 | 43.49 |
| ATOM | 1480 | CA | GLN | A | 156 | 157.323 | 32.475 | 1.398 | 1.00 | 44.17 |
| ATOM | 1481 | CB | GLN | A | 156 | 158.708 | 32.368 | 0.760 | 1.00 | 48.67 |
| ATOM | 1482 | CG | GLN | A | 156 | 159.097 | 30.954 | 0.310 | 1.00 | 55.67 |
| ATOM | 1483 | CD | GLN | A | 156 | 160.369 | 30.440 | 0.988 | 1.00 | 59.91 |
| ATOM | 1484 | OE1 | GLN | A | 156 | 160.673 | 30.802 | 2.125 | 1.00 | 61.96 |
| ATOM | 1485 | NE2 | GLN | A | 156 | 161.114 | 29.593 | 0.285 | 1.00 | 60.94 |
| ATOM | 1488 | C | GLN | A | 156 | 157.163 | 31.384 | 2.452 | 1.00 | 43.64 |
| ATOM | 1489 | O | GLN | A | 156 | 156.445 | 30.408 | 2.249 | 1.00 | 43.84 |
| ATOM | 1490 | N | LEU | A | 157 | 157.834 | 31.581 | 3.585 | 1.00 | 43.44 |
| ATOM | 1492 | CA | LEU | A | 157 | 157.794 | 30.653 | 4.704 | 1.00 | 41.27 |
| ATOM | 1493 | CB | LEU | A | 157 | 158.644 | 31.175 | 5.866 | 1.00 | 42.44 |
| ATOM | 1494 | CG | LEU | A | 157 | 160.152 | 30.930 | 5.797 | 1.00 | 42.39 |
| ATOM | 1495 | CD1 | LEU | A | 157 | 160.873 | 31.722 | 6.876 | 1.00 | 41.73 |
| ATOM | 1496 | CD2 | LEU | A | 157 | 160.426 | 29.447 | 5.948 | 1.00 | 42.15 |
| ATOM | 1497 | C | LEU | A | 157 | 156.372 | 30.423 | 5.173 | 1.00 | 40.27 |
| ATOM | 1498 | O | LEU | A | 157 | 155.948 | 29.280 | 5.328 | 1.00 | 41.05 |
| ATOM | 1499 | N | PHE | A | 158 | 155.620 | 31.497 | 5.378 | 1.00 | 39.18 |
| ATOM | 1501 | CA | PHE | A | 158 | 154.246 | 31.341 | 5.821 | 1.00 | 41.38 |
| ATOM | 1502 | CB | PHE | A | 158 | 153.519 | 32.684 | 5.927 | 1.00 | 40.94 |
| ATOM | 1503 | CG | PHE | A | 158 | 152.145 | 32.570 | 6.537 | 1.00 | 43.98 |
| ATOM | 1504 | CD1 | PHE | A | 158 | 151.068 | 32.087 | 5.788 | 1.00 | 44.07 |
| ATOM | 1505 | CD2 | PHE | A | 158 | 151.939 | 32.886 | 7.878 | 1.00 | 43.45 |
| ATOM | 1506 | CE1 | PHE | A | 158 | 149.814 | 31.915 | 6.367 | 1.00 | 42.96 |
| ATOM | 1507 | CE2 | PHE | A | 158 | 150.690 | 32.718 | 8.465 | 1.00 | 41.63 |
| ATOM | 1508 | CZ | PHE | A | 158 | 149.626 | 32.231 | 7.707 | 1.00 | 43.20 |
| ATOM | 1509 | C | PHE | A | 158 | 153.472 | 30.409 | 4.893 | 1.00 | 42.96 |
| ATOM | 1510 | O | PHE | A | 158 | 152.862 | 29.443 | 5.350 | 1.00 | 44.53 |
| ATOM | 1511 | N | HIS | A | 159 | 153.513 | 30.682 | 3.594 | 1.00 | 45.12 |
| ATOM | 1513 | CA | HIS | A | 159 | 152.790 | 29.860 | 2.624 | 1.00 | 46.02 |
| ATOM | 1514 | CB | HIS | A | 159 | 152.808 | 30.507 | 1.230 | 1.00 | 45.21 |
| ATOM | 1515 | CG | HIS | A | 159 | 152.049 | 31.796 | 1.145 | 1.00 | 44.03 |
| ATOM | 1516 | CD2 | HIS | A | 159 | 152.477 | 33.081 | 1.099 | 1.00 | 44.13 |
| ATOM | 1517 | ND1 | HIS | A | 159 | 150.674 | 31.851 | 1.087 | 1.00 | 44.17 |
| ATOM | 1519 | CE1 | HIS | A | 159 | 150.285 | 33.111 | 1.012 | 1.00 | 45.07 |
| ATOM | 1520 | NE2 | HIS | A | 159 | 151.360 | 33.878 | 1.016 | 1.00 | 45.02 |
| ATOM | 1522 | C | HIS | A | 159 | 153.314 | 28.426 | 2.524 | 1.00 | 47.97 |
| ATOM | 1523 | O | HIS | A | 159 | 152.530 | 27.491 | 2.353 | 1.00 | 48.86 |
| ATOM | 1524 | N | LYS | A | 160 | 154.624 | 28.253 | 2.678 | 1.00 | 49.77 |
| ATOM | 1526 | CA | LYS | A | 160 | 155.246 | 26.935 | 2.562 | 1.00 | 50.79 |
| ATOM | 1527 | CB | LYS | A | 160 | 156.664 | 27.080 | 1.990 | 1.00 | 51.90 |
| ATOM | 1528 | CG | LYS | A | 160 | 157.285 | 25.768 | 1.539 | 1.00 | 53.77 |
| ATOM | 1529 | CD | LYS | A | 160 | 158.669 | 25.957 | 0.950 | 1.00 | 54.99 |
| ATOM | 1530 | CE | LYS | A | 160 | 159.130 | 24.690 | 0.231 | 1.00 | 57.16 |
| ATOM | 1531 | NZ | LYS | A | 160 | 158.253 | 24.330 | −0.934 | 1.00 | 55.19 |
| ATOM | 1535 | C | LYS | A | 160 | 155.286 | 26.065 | 3.825 | 1.00 | 51.12 |
| ATOM | 1536 | O | LYS | A | 160 | 155.537 | 24.862 | 3.736 | 1.00 | 52.93 |
| ATOM | 1537 | N | LYS | A | 161 | 155.031 | 26.646 | 4.991 | 1.00 | 49.90 |
| ATOM | 1539 | CA | LYS | A | 161 | 155.086 | 25.873 | 6.229 | 1.00 | 48.78 |
| ATOM | 1540 | CB | LYS | A | 161 | 156.340 | 26.263 | 7.016 | 1.00 | 51.36 |
| ATOM | 1541 | CG | LYS | A | 161 | 157.660 | 25.983 | 6.310 | 1.00 | 55.24 |
| ATOM | 1542 | CD | LYS | A | 161 | 158.186 | 24.592 | 6.626 | 1.00 | 59.40 |
| ATOM | 1543 | CE | LYS | A | 161 | 158.484 | 24.443 | 8.114 | 1.00 | 61.04 |
| ATOM | 1544 | NZ | LYS | A | 161 | 159.015 | 23.095 | 8.464 | 1.00 | 62.60 |
| ATOM | 1548 | C | LYS | A | 161 | 153.875 | 26.012 | 7.149 | 1.00 | 47.20 |
| ATOM | 1549 | O | LYS | A | 161 | 153.460 | 25.042 | 7.787 | 1.00 | 46.35 |
| ATOM | 1550 | N | ILE | A | 162 | 153.311 | 27.214 | 7.209 | 1.00 | 46.07 |
| ATOM | 1552 | CA | ILE | A | 162 | 152.186 | 27.506 | 8.094 | 1.00 | 45.42 |
| ATOM | 1553 | CB | ILE | A | 162 | 152.421 | 28.854 | 8.806 | 1.00 | 45.05 |
| ATOM | 1554 | CG2 | ILE | A | 162 | 151.211 | 29.251 | 9.646 | 1.00 | 45.73 |
| ATOM | 1555 | CG1 | ILE | A | 162 | 153.679 | 28.764 | 9.670 | 1.00 | 43.35 |
| ATOM | 1556 | CD1 | ILE | A | 162 | 154.015 | 30.042 | 10.353 | 1.00 | 43.37 |
| ATOM | 1557 | C | ILE | A | 162 | 150.764 | 27.483 | 7.520 | 1.00 | 45.58 |
| ATOM | 1558 | O | ILE | A | 162 | 149.849 | 26.960 | 8.157 | 1.00 | 45.52 |
| ATOM | 1559 | N | GLU | A | 163 | 150.570 | 28.036 | 6.329 | 1.00 | 46.35 |
| ATOM | 1561 | CA | GLU | A | 163 | 149.239 | 28.093 | 5.731 | 1.00 | 47.82 |
| ATOM | 1562 | CB | GLU | A | 153 | 149.310 | 28.624 | 4.302 | 1.00 | 49.25 |
| ATOM | 1563 | CG | GLU | A | 163 | 147.956 | 29.028 | 3.735 | 1.00 | 51.68 |
| ATOM | 1564 | CD | GLU | A | 163 | 147.965 | 29.204 | 2.224 | 1.00 | 52.54 |
| ATOM | 1565 | OE1 | GLU | A | 163 | 149.007 | 29.597 | 1.658 | 1.00 | 53.57 |
| ATOM | 1566 | OE2 | GLU | A | 163 | 146.918 | 28.941 | 1.599 | 1.00 | 53.52 |
| ATOM | 1567 | C | GLU | A | 163 | 148.470 | 26.775 | 5.746 | 1.00 | 47.94 |
| ATOM | 1568 | O | GLU | A | 163 | 147.289 | 26.757 | 6.069 | 1.00 | 49.00 |
| ATOM | 1569 | N | SER | A | 164 | 149.142 | 25.678 | 5.420 | 1.00 | 49.09 |
| ATOM | 1571 | CA | SER | A | 164 | 148.510 | 24.360 | 5.383 | 1.00 | 49.75 |
| ATOM | 1572 | CB | SER | A | 164 | 149.486 | 23.327 | 4.818 | 1.00 | 50.94 |
| ATOM | 1573 | OG | SER | A | 164 | 149.971 | 23.737 | 3.548 | 1.00 | 56.63 |
| ATOM | 1575 | C | SER | A | 164 | 147.953 | 23.865 | 6.717 | 1.00 | 49.89 |
| ATOM | 1576 | O | SER | A | 164 | 147.100 | 22.982 | 6.739 | 1.00 | 52.82 |
| ATOM | 1577 | N | ALA | A | 165 | 148.442 | 24.415 | 7.824 | 1.00 | 48.05 |
| ATOM | 1579 | CA | ALA | A | 165 | 147.978 | 24.017 | 9.151 | 1.00 | 44.58 |
| ATOM | 1580 | CB | ALA | A | 165 | 149.153 | 23.920 | 10.109 | 1.00 | 45.11 |
| ATOM | 1581 | C | ALA | A | 165 | 146.947 | 25.009 | 9.678 | 1.00 | 43.87 |
| ATOM | 1582 | O | ALA | A | 165 | 145.980 | 24.626 | 10.335 | 1.00 | 43.62 |
| ATOM | 1583 | N | LEU | A | 166 | 147.169 | 26.287 | 9.391 | 1.00 | 43.88 |
| ATOM | 1585 | CA | LEU | A | 166 | 146.267 | 27.349 | 9.812 | 1.00 | 44.57 |
| ATOM | 1586 | CB | LEU | A | 166 | 146.783 | 28.701 | 9.313 | 1.00 | 41.17 |
| ATOM | 1587 | CG | LEU | A | 166 | 145.977 | 29.957 | 9.655 | 1.00 | 39.43 |
| ATOM | 1588 | CD1 | LEU | A | 166 | 146.438 | 30.508 | 10.979 | 1.00 | 40.32 |
| ATOM | 1589 | CD2 | LEU | A | 166 | 146.165 | 31.005 | 8.577 | 1.00 | 39.33 |
| ATOM | 1590 | C | LEU | A | 166 | 144.870 | 27.094 | 9.246 | 1.00 | 47.80 |
| ATOM | 1591 | O | LEU | A | 166 | 143.899 | 27.003 | 9.995 | 1.00 | 50.31 |
| ATOM | 1592 | N | ARG | A | 167 | 144.777 | 26.946 | 7.927 | 1.00 | 49.83 |
| ATOM | 1594 | CA | ARG | A | 167 | 143.493 | 26.714 | 7.274 | 1.00 | 53.68 |
| ATOM | 1595 | CB | ARG | A | 167 | 143.659 | 26.564 | 5.753 | 1.00 | 54.31 |
| ATOM | 1596 | CG | ARG | A | 167 | 144.488 | 25.354 | 5.329 | 1.00 | 57.76 |
| ATOM | 1597 | CD | ARG | A | 167 | 144.282 | 24.966 | 3.869 | 1.00 | 58.03 |
| ATOM | 1598 | NE | ARG | A | 167 | 144.657 | 26.032 | 2.948 | 1.00 | 56.39 |
| ATOM | 1600 | CZ | ARG | A | 167 | 143.786 | 26.753 | 2.256 | 1.00 | 55.99 |
| ATOM | 1601 | NH1 | ARG | A | 167 | 142.485 | 26.525 | 2.380 | 1.00 | 56.36 |
| ATOM | 1604 | NH2 | ARG | A | 167 | 144.214 | 27.699 | 1.436 | 1.00 | 55.25 |
| ATOM | 1607 | C | ARG | A | 167 | 142.774 | 25.495 | 7.836 | 1.00 | 56.26 |
| ATOM | 1608 | O | ARG | A | 167 | 141.570 | 25.544 | 8.088 | 1.00 | 58.47 |
| ATOM | 1609 | N | ASN | A | 168 | 143.517 | 24.418 | 8.077 | 1.00 | 59.06 |
| ATOM | 1611 | CA | ASN | A | 168 | 142.924 | 23.188 | 8.596 | 1.00 | 60.42 |
| ATOM | 1612 | CB | ASN | A | 168 | 143.936 | 22.037 | 8.586 | 1.00 | 63.69 |
| ATOM | 1613 | CG | ASN | A | 168 | 143.264 | 20.670 | 8.594 | 1.00 | 69.44 |
| ATOM | 1614 | OD1 | ASN | A | 168 | 142.186 | 20.491 | 8.021 | 1.00 | 73.41 |
| ATOM | 1615 | ND2 | ASN | A | 168 | 143.902 | 19.696 | 9.232 | 1.00 | 71.02 |
| ATOM | 1618 | C | ASN | A | 168 | 142.358 | 23.378 | 9.995 | 1.00 | 58.54 |
| ATOM | 1619 | O | ASN | A | 168 | 141.291 | 22.857 | 10.312 | 1.00 | 59.34 |
| ATOM | 1620 | N | LYS | A | 169 | 143.059 | 24.138 | 10.826 | 1.00 | 56.61 |
| ATOM | 1622 | CA | LYS | A | 169 | 142.591 | 24.380 | 12.179 | 1.00 | 54.57 |
| ATOM | 1623 | CB | LYS | A | 169 | 143.732 | 24.882 | 13.065 | 1.00 | 56.85 |
| ATOM | 1624 | CG | LYS | A | 169 | 143.365 | 25.002 | 14.540 | 1.00 | 60.65 |
| ATOM | 1625 | CD | LYS | A | 169 | 144.599 | 24.927 | 15.425 | 1.00 | 65.37 |
| ATOM | 1626 | CE | LYS | A | 169 | 145.276 | 23.560 | 15.329 | 1.00 | 68.82 |
| ATOM | 1627 | NZ | LYS | A | 169 | 146.576 | 23.504 | 16.062 | 1.00 | 70.63 |
| ATOM | 1631 | C | LYS | A | 169 | 141.439 | 25.372 | 12.173 | 1.00 | 51.59 |
| ATOM | 1632 | O | LYS | A | 169 | 140.570 | 25.327 | 13.035 | 1.00 | 51.73 |
| ATOM | 1633 | N | MET | A | 170 | 141.426 | 26.264 | 11.192 | 1.00 | 49.16 |
| ATOM | 1635 | CA | MET | A | 170 | 140.362 | 27.251 | 11.090 | 1.00 | 47.96 |
| ATOM | 1636 | CB | MET | A | 170 | 140.733 | 28.340 | 10.087 | 1.00 | 49.44 |
| ATOM | 1637 | CG | MET | A | 170 | 139.792 | 29.528 | 10.105 | 1.00 | 52.89 |
| ATOM | 1638 | SD | MET | A | 170 | 139.730 | 30.289 | 11.734 | 1.00 | 55.23 |
| ATOM | 1639 | CE | MET | A | 170 | 141.170 | 31.310 | 11.695 | 1.00 | 55.26 |
| ATOM | 1640 | C | MET | A | 170 | 139.075 | 26.565 | 10.659 | 1.00 | 45.37 |
| ATOM | 1641 | O | MET | A | 170 | 137.989 | 26.892 | 11.139 | 1.00 | 46.51 |
| ATOM | 1642 | N | ASN | A | 171 | 139.207 | 25.601 | 9.759 | 1.00 | 42.65 |
| ATOM | 1644 | CA | ASN | A | 171 | 138.063 | 24.858 | 9.268 | 1.00 | 41.99 |
| ATOM | 1645 | CB | ASN | A | 171 | 138.448 | 24.034 | 8.039 | 1.00 | 42.92 |
| ATOM | 1646 | CG | ASN | A | 171 | 138.644 | 24.889 | 6.795 | 1.00 | 43.47 |
| ATOM | 1647 | OD1 | ASN | A | 171 | 138.041 | 25.957 | 6.658 | 1.00 | 44.24 |
| ATOM | 1648 | ND2 | ASN | A | 171 | 139.478 | 24.415 | 5.875 | 1.00 | 44.77 |
| ATOM | 1651 | C | ASN | A | 171 | 137.487 | 23.962 | 10.353 | 1.00 | 42.70 |
| ATOM | 1652 | O | ASN | A | 171 | 136.284 | 23.701 | 10.374 | 1.00 | 43.68 |
| ATOM | 1653 | N | SER | A | 172 | 138.341 | 23.502 | 11.263 | 1.00 | 44.20 |
| ATOM | 1655 | CA | SER | A | 172 | 137.898 | 22.645 | 12.358 | 1.00 | 45.11 |
| ATOM | 1656 | CB | SER | A | 172 | 139.069 | 21.841 | 12.935 | 1.00 | 46.36 |
| ATOM | 1657 | OG | SER | A | 172 | 140.094 | 22.685 | 13.438 | 1.00 | 49.96 |
| ATOM | 1659 | C | SER | A | 172 | 137.230 | 23.474 | 13.454 | 1.00 | 44.96 |
| ATOM | 1660 | O | SER | A | 172 | 136.204 | 23.069 | 14.005 | 1.00 | 47.71 |
| ATOM | 1661 | N | GLN | A | 173 | 137.811 | 24.635 | 13.759 | 1.00 | 43.91 |
| ATOM | 1663 | CA | GLN | A | 173 | 137.276 | 25.541 | 14.776 | 1.00 | 40.36 |
| ATOM | 1664 | CB | GLN | A | 173 | 138.214 | 26.730 | 14.990 | 1.00 | 41.61 |
| ATOM | 1665 | CG | GLN | A | 173 | 139.444 | 26.421 | 15.816 | 1.00 | 43.62 |
| ATOM | 1666 | CD | GLN | A | 173 | 139.095 | 25.943 | 17.205 | 1.00 | 46.78 |
| ATOM | 1667 | OE1 | GLN | A | 173 | 138.657 | 26.722 | 18.052 | 1.00 | 50.29 |
| ATOM | 1668 | NE2 | GLN | A | 173 | 139.279 | 24.654 | 17.448 | 1.00 | 48.32 |
| ATOM | 1671 | C | GLN | A | 173 | 135.893 | 26.047 | 14.386 | 1.00 | 38.81 |
| ATOM | 1672 | O | GLN | A | 173 | 134.990 | 26.098 | 15.218 | 1.00 | 38.42 |
| ATOM | 1673 | N | VAL | A | 174 | 135.734 | 26.423 | 13.120 | 1.00 | 36.59 |
| ATOM | 1675 | CA | VAL | A | 174 | 134.456 | 26.912 | 12.621 | 1.00 | 34.47 |
| ATOM | 1676 | CB | VAL | A | 174 | 134.559 | 27.328 | 11.147 | 1.00 | 32.46 |
| ATOM | 1677 | CG1 | VAL | A | 174 | 133.183 | 27.559 | 10.564 | 1.00 | 32.42 |
| ATOM | 1678 | CG2 | VAL | A | 174 | 135.380 | 28.601 | 11.037 | 1.00 | 31.13 |
| ATOM | 1679 | C | VAL | A | 174 | 133.363 | 25.861 | 12.810 | 1.00 | 34.32 |
| ATOM | 1680 | O | VAL | A | 174 | 132.350 | 26.130 | 13.445 | 1.00 | 36.14 |
| ATOM | 1681 | N | CYS | A | 175 | 133.586 | 24.652 | 12.308 | 1.00 | 35.02 |
| ATOM | 1683 | CA | CYS | A | 175 | 132.601 | 23.591 | 12.460 | 1.00 | 35.40 |
| ATOM | 1684 | C | CYS | A | 175 | 132.440 | 23.152 | 13.906 | 1.00 | 36.62 |
| ATOM | 1685 | O | CYS | A | 175 | 131.405 | 22.597 | 14.284 | 1.00 | 37.92 |
| ATOM | 1686 | CB | CYS | A | 175 | 132.925 | 22.396 | 11.573 | 1.00 | 33.94 |
| ATOM | 1687 | SG | CYS | A | 175 | 132.302 | 22.595 | 9.876 | 1.00 | 42.37 |
| ATOM | 1688 | N | GLU | A | 176 | 133.459 | 23.386 | 14.720 | 1.00 | 38.42 |
| ATOM | 1690 | CA | GLU | A | 176 | 133.365 | 23.034 | 16.123 | 1.00 | 40.14 |
| ATOM | 1691 | CB | GLU | A | 176 | 134.739 | 23.093 | 16.793 | 1.00 | 43.35 |
| ATOM | 1692 | CG | GLU | A | 176 | 134.747 | 22.502 | 18.193 | 1.00 | 50.93 |
| ATOM | 1693 | CD | GLU | A | 176 | 136.132 | 22.385 | 18.783 | 1.00 | 53.93 |
| ATOM | 1694 | OE1 | GLU | A | 176 | 136.867 | 21.459 | 18.382 | 1.00 | 58.04 |
| ATOM | 1695 | OE2 | GLU | A | 176 | 136.481 | 23.207 | 19.658 | 1.00 | 57.02 |
| ATOM | 1696 | C | GLU | A | 176 | 132.386 | 24.024 | 16.772 | 1.00 | 40.43 |
| ATOM | 1697 | O | GLU | A | 176 | 131.498 | 23.627 | 17.528 | 1.00 | 41.95 |
| ATOM | 1698 | N | LYS | A | 177 | 132.507 | 25.301 | 16.419 | 1.00 | 39.36 |
| ATOM | 1700 | CA | LYS | A | 177 | 131.625 | 26.332 | 16.956 | 1.00 | 38.66 |
| ATOM | 1701 | CB | LYS | A | 177 | 132.074 | 27.729 | 16.510 | 1.00 | 38.94 |
| ATOM | 1702 | CG | LYS | A | 177 | 133.403 | 28.194 | 17.089 | 1.00 | 40.75 |
| ATOM | 1703 | CD | LYS | A | 177 | 133.336 | 28.424 | 18.592 | 1.00 | 40.88 |
| ATOM | 1704 | CE | LYS | A | 177 | 134.694 | 28.852 | 19.152 | 1.00 | 40.13 |
| ATOM | 1705 | NZ | LYS | A | 177 | 134.688 | 28.995 | 20.637 | 1.00 | 40.04 |
| ATOM | 1709 | C | LYS | A | 177 | 130.190 | 26.099 | 16.503 | 1.00 | 37.24 |
| ATOM | 1710 | O | LYS | A | 177 | 129.277 | 26.078 | 17.318 | 1.00 | 39.39 |
| ATOM | 1711 | N | VAL | A | 178 | 130.002 | 25.901 | 15.204 | 1.00 | 35.46 |
| ATOM | 1713 | CA | VAL | A | 178 | 128.676 | 25.676 | 14.641 | 1.00 | 33.61 |
| ATOM | 1714 | CB | VAL | A | 178 | 128.747 | 25.442 | 13.124 | 1.00 | 31.21 |
| ATOM | 1715 | CD1 | VAL | A | 178 | 127.365 | 25.166 | 12.568 | 1.00 | 32.70 |
| ATOM | 1716 | CG2 | VAL | A | 178 | 129.346 | 26.652 | 12.446 | 1.00 | 31.16 |
| ATOM | 1717 | C | VAL | A | 178 | 127.960 | 24.505 | 15.299 | 1.00 | 34.02 |
| ATOM | 1718 | O | VAL | A | 178 | 126.844 | 24.656 | 15.784 | 1.00 | 36.09 |
| ATOM | 1719 | N | THR | A | 179 | 128.611 | 23.349 | 15.338 | 1.00 | 34.04 |
| ATOM | 1721 | CA | THR | A | 179 | 128.018 | 22.167 | 15.947 | 1.00 | 35.60 |
| ATOM | 1722 | CB | THR | A | 179 | 128.977 | 20.968 | 15.865 | 1.00 | 32.77 |
| ATOM | 1723 | OG1 | THR | A | 179 | 129.372 | 20.775 | 14.505 | 1.00 | 32.73 |
| ATOM | 1725 | CG2 | THR | A | 179 | 128.296 | 19.706 | 16.330 | 1.00 | 35.18 |
| ATOM | 1726 | C | THR | A | 179 | 127.634 | 22.428 | 17.408 | 1.00 | 39.92 |
| ATOM | 1727 | O | THR | A | 179 | 126.554 | 22.037 | 17.858 | 1.00 | 42.33 |
| ATOM | 1728 | N | ASN | A | 180 | 128.497 | 23.131 | 18.132 | 1.00 | 43.69 |
| ATOM | 1730 | CA | ASN | A | 180 | 128.244 | 23.435 | 19.537 | 1.00 | 44.92 |
| ATOM | 1731 | CB | ASN | A | 180 | 129.496 | 23.991 | 20.213 | 1.00 | 49.20 |
| ATOM | 1732 | CG | ASN | A | 180 | 130.522 | 22.920 | 20.532 | 1.00 | 53.34 |
| ATOM | 1733 | OD1 | ASN | A | 180 | 131.550 | 23.213 | 21.140 | 1.00 | 58.92 |
| ATOM | 1734 | ND2 | ASN | A | 180 | 130.258 | 21.680 | 20.126 | 1.00 | 53.15 |
| ATOM | 1737 | C | ASN | A | 180 | 127.101 | 24.406 | 19.738 | 1.00 | 44.35 |
| ATOM | 1738 | O | ASN | A | 180 | 126.252 | 24.194 | 20.593 | 1.00 | 46.90 |
| ATOM | 1739 | N | SER | A | 181 | 127.095 | 25.487 | 18.972 | 1.00 | 42.77 |
| ATOM | 1741 | CA | SER | A | 181 | 126.047 | 26.481 | 19.084 | 1.00 | 42.55 |
| ATOM | 1742 | CB | SER | A | 181 | 126.274 | 27.605 | 18.076 | 1.00 | 46.25 |
| ATOM | 1743 | OG | SER | A | 181 | 127.508 | 28.261 | 18.319 | 1.00 | 51.45 |
| ATOM | 1745 | C | SER | A | 181 | 124.686 | 25.841 | 18.869 | 1.00 | 41.46 |
| ATOM | 1746 | O | SER | A | 181 | 123.720 | 26.203 | 19.528 | 1.00 | 43.31 |
| ATOM | 1747 | N | VAL | A | 182 | 124.610 | 24.870 | 17.969 | 1.00 | 41.03 |
| ATOM | 1749 | CA | VAL | A | 182 | 123.346 | 24.195 | 17.712 | 1.00 | 40.50 |
| ATOM | 1750 | CB | VAL | A | 182 | 123.468 | 23.177 | 16.565 | 1.00 | 40.39 |
| ATOM | 1751 | CG1 | VAL | A | 182 | 122.133 | 22.489 | 16.333 | 1.00 | 41.54 |
| ATOM | 1752 | CG2 | VAL | A | 182 | 123.918 | 23.874 | 15.295 | 1.00 | 39.67 |
| ATOM | 1753 | C | VAL | A | 182 | 122.865 | 23.481 | 18.970 | 1.00 | 41.04 |
| ATOM | 1754 | O | VAL | A | 182 | 121.831 | 23.836 | 19.536 | 1.00 | 41.32 |
| ATOM | 1755 | N | SER | A | 183 | 123.647 | 22.510 | 19.429 | 1.00 | 42.02 |
| ATOM | 1757 | CA | SER | A | 183 | 123.311 | 21.735 | 20.616 | 1.00 | 41.82 |
| ATOM | 1758 | CB | SER | A | 183 | 124.339 | 20.620 | 20.828 | 1.00 | 42.01 |
| ATOM | 1759 | OG | SER | A | 183 | 124.180 | 19.588 | 19.867 | 1.00 | 47.44 |
| ATOM | 1761 | C | SER | A | 183 | 123.187 | 22.553 | 21.895 | 1.00 | 41.66 |
| ATOM | 1762 | O | SER | A | 183 | 122.251 | 22.358 | 22.673 | 1.00 | 43.45 |
| ATOM | 1763 | N | SER | A | 184 | 124.120 | 23.472 | 22.102 | 1.00 | 40.24 |
| ATOM | 1765 | CA | SER | A | 184 | 124.143 | 24.293 | 23.305 | 1.00 | 40.26 |
| ATOM | 1766 | CB | SER | A | 184 | 125.590 | 24.639 | 23.670 | 1.00 | 42.61 |
| ATOM | 1767 | OG | SER | A | 184 | 126.340 | 23.468 | 23.947 | 1.00 | 47.85 |
| ATOM | 1769 | C | SER | A | 184 | 123.307 | 25.567 | 23.312 | 1.00 | 39.22 |
| ATOM | 1770 | O | SER | A | 184 | 122.991 | 26.088 | 24.382 | 1.00 | 40.56 |
| ATOM | 1771 | N | GLU | A | 185 | 122.942 | 26.078 | 22.145 | 1.00 | 37.87 |
| ATOM | 1773 | CA | GLU | A | 185 | 122.169 | 27.313 | 22.098 | 1.00 | 37.71 |
| ATOM | 1774 | CB | GLU | A | 185 | 123.060 | 28.485 | 21.683 | 1.00 | 40.53 |
| ATOM | 1775 | CG | GLU | A | 185 | 124.010 | 28.960 | 22.768 | 1.00 | 45.79 |
| ATOM | 1776 | CD | GLU | A | 185 | 124.987 | 30.024 | 22.296 | 1.00 | 50.89 |
| ATOM | 1777 | OE1 | GLU | A | 185 | 124.887 | 30.490 | 21.138 | 1.00 | 55.20 |
| ATOM | 1778 | OE2 | GLU | A | 185 | 125.872 | 30.390 | 23.097 | 1.00 | 55.19 |
| ATOM | 1779 | C | GLU | A | 185 | 120.934 | 27.277 | 21.225 | 1.00 | 35.83 |
| ATOM | 1780 | O | GLU | A | 185 | 119.921 | 27.861 | 21.584 | 1.00 | 38.77 |
| ATOM | 1781 | N | LEU | A | 186 | 121.008 | 26.605 | 20.082 | 1.00 | 34.44 |
| ATOM | 1783 | CA | LEU | A | 186 | 119.864 | 26.533 | 19.187 | 1.00 | 33.39 |
| ATOM | 1784 | CB | LEU | A | 186 | 120.269 | 25.978 | 17.818 | 1.00 | 30.92 |
| ATOM | 1785 | CG | LEU | A | 186 | 119.709 | 26.645 | 16.553 | 1.00 | 26.02 |
| ATOM | 1786 | CD1 | LEU | A | 186 | 119.228 | 25.581 | 15.601 | 1.00 | 23.97 |
| ATOM | 1787 | CD2 | LEU | A | 186 | 118.581 | 27.619 | 16.867 | 1.00 | 25.38 |
| ATOM | 1788 | C | LEU | A | 186 | 118.794 | 25.652 | 19.802 | 1.00 | 34.20 |
| ATOM | 1789 | O | LEU | A | 186 | 117.655 | 26.083 | 19.963 | 1.00 | 35.35 |
| ATOM | 1790 | N | GLN | A | 187 | 119.168 | 24.433 | 20.181 | 1.00 | 35.06 |
| ATOM | 1792 | CA | GLN | A | 187 | 118.218 | 23.500 | 20.778 | 1.00 | 37.32 |
| ATOM | 1793 | CB | GLN | A | 187 | 118.856 | 22.130 | 21.031 | 1.00 | 38.08 |
| ATOM | 1794 | CG | GLN | A | 187 | 117.859 | 21.104 | 21.549 | 1.00 | 39.72 |
| ATOM | 1795 | CD | GLN | A | 187 | 118.388 | 19.696 | 21.506 | 1.00 | 39.64 |
| ATOM | 1796 | OE1 | GLN | A | 187 | 118.203 | 18.984 | 20.520 | 1.00 | 42.24 |
| ATOM | 1797 | NE2 | GLN | A | 187 | 119.031 | 19.272 | 22.585 | 1.00 | 43.70 |
| ATOM | 1800 | C | GLN | A | 187 | 117.548 | 24.041 | 22.041 | 1.00 | 36.63 |
| ATOM | 1801 | O | GLN | A | 187 | 116.325 | 24.022 | 22.138 | 1.00 | 40.27 |
| ATOM | 1802 | N | PRO | A | 188 | 118.333 | 24.493 | 23.038 | 1.00 | 36.32 |
| ATOM | 1803 | CD | PRO | A | 188 | 119.782 | 24.293 | 23.223 | 1.00 | 38.65 |
| ATOM | 1804 | CA | PRO | A | 188 | 117.748 | 25.033 | 24.268 | 1.00 | 35.72 |
| ATOM | 1805 | CB | PRO | A | 188 | 118.977 | 25.432 | 25.075 | 1.00 | 34.46 |
| ATOM | 1806 | CG | PRO | A | 188 | 119.933 | 24.364 | 24.730 | 1.00 | 35.29 |
| ATOM | 1807 | C | PRO | A | 188 | 116.829 | 26.231 | 24.030 | 1.00 | 36.20 |
| ATOM | 1808 | O | PRO | A | 188 | 115.933 | 26.490 | 24.835 | 1.00 | 38.62 |
| ATOM | 1809 | N | TYR | A | 189 | 117.062 | 26.983 | 22.957 | 1.00 | 35.08 |
| ATOM | 1811 | CA | TYR | A | 189 | 116.203 | 28.122 | 22.667 | 1.00 | 33.10 |
| ATOM | 1812 | CB | TYR | A | 189 | 116.799 | 29.059 | 21.625 | 1.00 | 30.49 |
| ATOM | 1813 | CG | TYR | A | 189 | 115.767 | 30.037 | 21.113 | 1.00 | 29.52 |
| ATOM | 1814 | CD1 | TYR | A | 189 | 115.222 | 30.996 | 21.958 | 1.00 | 28.37 |
| ATOM | 1815 | CE1 | TYR | A | 189 | 114.211 | 31.842 | 21.523 | 1.00 | 27.60 |
| ATOM | 1816 | CD2 | TYR | A | 189 | 115.274 | 29.950 | 19.808 | 1.00 | 28.40 |
| ATOM | 1817 | CE2 | TYR | A | 189 | 114.261 | 30.792 | 19.366 | 1.00 | 26.35 |
| ATOM | 1818 | CZ | TYR | A | 189 | 113.737 | 31.732 | 20.231 | 1.00 | 25.96 |
| ATOM | 1819 | OH | TYR | A | 189 | 112.735 | 32.568 | 19.815 | 1.00 | 27.51 |
| ATOM | 1821 | C | TYR | A | 189 | 114.841 | 27.660 | 22.186 | 1.00 | 34.73 |
| ATOM | 1822 | O | TYR | A | 189 | 113.824 | 28.113 | 22.694 | 1.00 | 38.35 |
| ATOM | 1823 | N | PHE | A | 190 | 114.811 | 26.771 | 21.197 | 1.00 | 36.21 |
| ATOM | 1825 | CA | PHE | A | 190 | 113.538 | 26.288 | 20.684 | 1.00 | 37.47 |
| ATOM | 1826 | CB | PHE | A | 190 | 113.697 | 25.649 | 19.302 | 1.00 | 38.76 |
| ATOM | 1827 | CG | PHE | A | 190 | 113.522 | 26.633 | 18.177 | 1.00 | 41.54 |
| ATOM | 1828 | CD1 | PHE | A | 190 | 112.273 | 27.194 | 17.920 | 1.00 | 40.00 |
| ATOM | 1829 | CD2 | PHE | A | 190 | 114.610 | 27.042 | 17.406 | 1.00 | 43.58 |
| ATOM | 1830 | CE1 | PHE | A | 190 | 112.109 | 28.146 | 16.919 | 1.00 | 39.03 |
| ATOM | 1831 | CE2 | PHE | A | 190 | 114.454 | 27.999 | 16.398 | 1.00 | 40.39 |
| ATOM | 1832 | CZ | PHE | A | 190 | 113.201 | 28.550 | 16.159 | 1.00 | 39.28 |
| ATOM | 1833 | C | PHE | A | 190 | 112.768 | 25.408 | 21.662 | 1.00 | 37.92 |
| ATOM | 1834 | O | PHE | A | 190 | 111.655 | 24.960 | 21.380 | 1.00 | 38.40 |
| ATOM | 1835 | N | GLN | A | 191 | 113.351 | 25.193 | 22.835 | 1.00 | 36.94 |
| ATOM | 1837 | CA | GLN | A | 191 | 112.685 | 24.425 | 23.869 | 1.00 | 36.24 |
| ATOM | 1838 | CB | GLN | A | 191 | 113.637 | 23.426 | 24.529 | 1.00 | 36.06 |
| ATOM | 1839 | CG | GLN | A | 191 | 113.987 | 22.259 | 23.609 | 1.00 | 37.66 |
| ATOM | 1840 | CD | GLN | A | 191 | 114.786 | 21.170 | 24.287 | 1.00 | 36.77 |
| ATOM | 1841 | OE1 | GLN | A | 191 | 115.365 | 21.379 | 25.349 | 1.00 | 38.68 |
| ATOM | 1842 | NE2 | GLN | A | 191 | 114.828 | 19.997 | 23.669 | 1.00 | 35.78 |
| ATOM | 1845 | C | GLN | A | 191 | 112.042 | 25.369 | 24.881 | 1.00 | 37.61 |
| ATOM | 1846 | O | GLN | A | 191 | 111.621 | 24.945 | 25.956 | 1.00 | 38.67 |
| ATOM | 1847 | N | THR | A | 192 | 111.992 | 26.659 | 24.541 | 1.00 | 38.44 |
| ATOM | 1849 | CA | THR | A | 192 | 111.338 | 27.650 | 25.392 | 1.00 | 37.16 |
| ATOM | 1850 | CB | THR | A | 192 | 111.946 | 29.055 | 25.263 | 1.00 | 36.82 |
| ATOM | 1851 | OG1 | THR | A | 192 | 111.937 | 29.461 | 23.890 | 1.00 | 37.79 |
| ATOM | 1853 | CG2 | THR | A | 192 | 113.357 | 29.083 | 25.802 | 1.00 | 36.99 |
| ATOM | 1854 | C | THR | A | 192 | 109.894 | 27.702 | 24.913 | 1.00 | 37.14 |
| ATOM | 1855 | O | THR | A | 192 | 109.082 | 28.485 | 25.403 | 1.00 | 38.53 |
| ATOM | 1856 | N | LEU | A | 193 | 109.604 | 26.884 | 23.906 | 1.00 | 38.31 |
| ATOM | 1858 | CA | LEU | A | 193 | 108.273 | 26.765 | 23.339 | 1.00 | 40.73 |
| ATOM | 1859 | CB | LEU | A | 193 | 108.325 | 25.772 | 22.174 | 1.00 | 42.93 |
| ATOM | 1860 | CG | LEU | A | 193 | 107.191 | 25.727 | 21.151 | 1.00 | 44.96 |
| ATOM | 1861 | CD1 | LEU | A | 193 | 107.000 | 27.100 | 20.518 | 1.00 | 45.48 |
| ATOM | 1862 | CD2 | LEU | A | 193 | 107.531 | 24.696 | 20.085 | 1.00 | 44.59 |
| ATOM | 1863 | C | LEU | A | 193 | 107.403 | 26.218 | 24.472 | 1.00 | 40.91 |
| ATOM | 1864 | O | LEU | A | 193 | 107.788 | 25.265 | 25.150 | 1.00 | 41.30 |
| ATOM | 1865 | N | PRO | A | 194 | 106.230 | 26.824 | 24.707 | 1.00 | 41.79 |
| ATOM | 1866 | CD | PRO | A | 194 | 105.632 | 27.950 | 23.974 | 1.00 | 41.39 |
| ATOM | 1867 | CA | PRO | A | 194 | 105.334 | 26.373 | 25.777 | 1.00 | 41.80 |
| ATOM | 1868 | CB | PRO | A | 194 | 104.217 | 27.420 | 25.754 | 1.00 | 42.00 |
| ATOM | 1869 | CG | PRO | A | 194 | 104.823 | 28.603 | 25.045 | 1.00 | 42.94 |
| ATOM | 1870 | C | PRO | A | 194 | 104.755 | 24.985 | 25.522 | 1.00 | 41.75 |
| ATOM | 1871 | O | PRO | A | 194 | 104.174 | 24.734 | 24.469 | 1.00 | 44.72 |
| ATOM | 1872 | N | VAL | A | 195 | 104.930 | 24.080 | 26.476 | 1.00 | 39.33 |
| ATOM | 1874 | CA | VAL | A | 195 | 104.383 | 22.738 | 26.338 | 1.00 | 37.64 |
| ATOM | 1875 | CB | VAL | A | 195 | 105.403 | 21.659 | 26.756 | 1.00 | 37.05 |
| ATOM | 1876 | CG1 | VAL | A | 195 | 104.744 | 20.295 | 26.840 | 1.00 | 34.56 |
| ATOM | 1877 | CG2 | VAL | A | 195 | 106.515 | 21.607 | 25.747 | 1.00 | 36.43 |
| ATOM | 1878 | C | VAL | A | 195 | 103.118 | 22.644 | 27.183 | 1.00 | 38.20 |
| ATOM | 1879 | O | VAL | A | 195 | 102.062 | 22.245 | 26.694 | 1.00 | 40.04 |
| ATOM | 1880 | N | MET | A | 196 | 103.234 | 23.007 | 28.454 | 1.00 | 38.05 |
| ATOM | 1882 | CA | MET | A | 196 | 102.103 | 22.987 | 29.370 | 1.00 | 37.71 |
| ATOM | 1883 | CB | MET | A | 196 | 102.414 | 22.128 | 30.592 | 1.00 | 40.52 |
| ATOM | 1884 | CG | MET | A | 196 | 102.756 | 20.681 | 30.283 | 1.00 | 43.85 |
| ATOM | 1885 | SD | MET | A | 196 | 101.356 | 19.738 | 29.695 | 1.00 | 42.31 |
| ATOM | 1886 | CE | MET | A | 196 | 100.644 | 19.226 | 31.232 | 1.00 | 41.10 |
| ATOM | 1887 | C | MET | A | 196 | 101.924 | 24.428 | 29.796 | 1.00 | 37.52 |
| ATOM | 1888 | O | MET | A | 196 | 102.521 | 24.877 | 30.771 | 1.00 | 40.72 |
| ATOM | 1889 | N | THR | A | 197 | 101.134 | 25.167 | 29.039 | 1.00 | 36.00 |
| ATOM | 1891 | CA | THR | A | 197 | 100.914 | 26.565 | 29.335 | 1.00 | 34.89 |
| ATOM | 1892 | CB | THR | A | 197 | 100.650 | 27.324 | 28.036 | 1.00 | 37.85 |
| ATOM | 1893 | OG1 | THR | A | 197 | 101.329 | 26.660 | 26.963 | 1.00 | 41.31 |
| ATOM | 1895 | CG2 | THR | A | 197 | 101.177 | 28.733 | 28.131 | 1.00 | 39.84 |
| ATOM | 1896 | C | THR | A | 197 | 99.751 | 26.772 | 30.302 | 1.00 | 32.85 |
| ATOM | 1897 | O | THR | A | 197 | 98.594 | 26.571 | 29.939 | 1.00 | 33.57 |
| ATOM | 1898 | N | LYS | A | 198 | 100.061 | 27.122 | 31.545 | 1.00 | 29.63 |
| ATOM | 1900 | CA | LYS | A | 198 | 99.036 | 27.372 | 32.551 | 1.00 | 27.13 |
| ATOM | 1901 | CB | LYS | A | 198 | 99.630 | 27.266 | 33.952 | 1.00 | 26.01 |
| ATOM | 1902 | CG | LYS | A | 198 | 98.609 | 27.379 | 35.047 | 1.00 | 26.97 |
| ATOM | 1903 | CD | LYS | A | 198 | 99.269 | 27.457 | 36.389 | 1.00 | 31.48 |
| ATOM | 1904 | CE | LYS | A | 198 | 98.232 | 27.489 | 37.482 | 1.00 | 33.31 |
| ATOM | 1905 | NZ | LYS | A | 198 | 97.393 | 26.263 | 37.448 | 1.00 | 38.94 |
| ATOM | 1909 | C | LYS | A | 198 | 98.555 | 28.789 | 32.307 | 1.00 | 26.60 |
| ATOM | 1910 | O | LYS | A | 198 | 99.365 | 29.706 | 32.264 | 1.00 | 27.53 |
| ATOM | 1911 | N | ILE | A | 199 | 97.248 | 28.975 | 32.154 | 1.00 | 26.62 |
| ATOM | 1913 | CA | ILE | A | 199 | 96.711 | 30.298 | 31.878 | 1.00 | 23.94 |
| ATOM | 1914 | CB | ILE | A | 199 | 95.817 | 30.283 | 30.613 | 1.00 | 25.54 |
| ATOM | 1915 | CG2 | ILE | A | 199 | 96.561 | 29.614 | 29.477 | 1.00 | 26.32 |
| ATOM | 1916 | CG1 | ILE | A | 199 | 94.543 | 29.487 | 30.843 | 1.00 | 23.67 |
| ATOM | 1917 | CD1 | ILE | A | 199 | 93.795 | 29.189 | 29.564 | 1.00 | 22.95 |
| ATOM | 1918 | C | ILE | A | 199 | 95.992 | 30.954 | 33.041 | 1.00 | 25.86 |
| ATOM | 1919 | O | ILE | A | 199 | 95.827 | 32.169 | 33.061 | 1.00 | 28.61 |
| ATOM | 1920 | N | ASP | A | 200 | 95.561 | 30.148 | 34.007 | 1.00 | 27.99 |
| ATOM | 1922 | CA | ASP | A | 200 | 94.878 | 30.651 | 35.197 | 1.00 | 26.92 |
| ATOM | 1923 | CB | ASP | A | 200 | 93.484 | 31.220 | 34.867 | 1.00 | 26.06 |
| ATOM | 1924 | CG | ASP | A | 200 | 92.582 | 30.234 | 34.151 | 1.00 | 24.60 |
| ATOM | 1925 | OD1 | ASP | A | 200 | 92.621 | 29.037 | 34.465 | 1.00 | 26.68 |
| ATOM | 1926 | OD2 | ASP | A | 200 | 91.813 | 30.664 | 33.271 | 1.00 | 28.99 |
| ATOM | 1927 | C | ASP | A | 200 | 94.805 | 29.589 | 36.289 | 1.00 | 27.44 |
| ATOM | 1928 | O | ASP | A | 200 | 95.509 | 28.591 | 36.225 | 1.00 | 29.73 |
| ATOM | 1929 | N | SER | A | 201 | 93.966 | 29.808 | 37.294 | 1.00 | 28.30 |
| ATOM | 1931 | CA | SER | A | 201 | 93.825 | 28.859 | 38.397 | 1.00 | 30.40 |
| ATOM | 1932 | CB | SER | A | 201 | 93.032 | 29.507 | 39.531 | 1.00 | 31.85 |
| ATOM | 1933 | OG | SER | A | 201 | 91.882 | 30.170 | 39.026 | 1.00 | 39.39 |
| ATOM | 1935 | C | SER | A | 201 | 93.137 | 27.562 | 38.000 | 1.00 | 29.79 |
| ATOM | 1936 | O | SER | A | 201 | 93.230 | 26.557 | 38.695 | 1.00 | 31.67 |
| ATOM | 1937 | N | VAL | A | 202 | 92.430 | 27.604 | 36.884 | 1.00 | 30.29 |
| ATOM | 1939 | CA | VAL | A | 202 | 91.683 | 26.457 | 36.409 | 1.00 | 29.89 |
| ATOM | 1940 | CB | VAL | A | 202 | 90.284 | 26.911 | 35.932 | 1.00 | 28.59 |
| ATOM | 1941 | CG1 | VAL | A | 202 | 89.426 | 25.723 | 35.568 | 1.00 | 30.45 |
| ATOM | 1942 | CG2 | VAL | A | 202 | 89.613 | 27.740 | 36.998 | 1.00 | 28.22 |
| ATOM | 1943 | C | VAL | A | 202 | 92.346 | 25.667 | 35.282 | 1.00 | 31.71 |
| ATOM | 1944 | O | VAL | A | 202 | 92.752 | 24.520 | 35.470 | 1.00 | 33.93 |
| ATOM | 1945 | N | ALA | A | 203 | 92.463 | 26.291 | 34.115 | 1.00 | 31.10 |
| ATOM | 1947 | CA | ALA | A | 203 | 92.997 | 25.621 | 32.944 | 1.00 | 29.58 |
| ATOM | 1948 | CB | ALA | A | 203 | 92.039 | 25.821 | 31.767 | 1.00 | 29.01 |
| ATOM | 1949 | C | ALA | A | 203 | 94.416 | 25.908 | 32.497 | 1.00 | 28.91 |
| ATOM | 1950 | O | ALA | A | 203 | 95.096 | 26.807 | 33.001 | 1.00 | 27.86 |
| ATOM | 1951 | N | GLY | A | 204 | 94.822 | 25.124 | 31.503 | 1.00 | 26.23 |
| ATOM | 1953 | CA | GLY | A | 204 | 96.124 | 25.227 | 30.894 | 1.00 | 22.94 |
| ATOM | 1954 | C | GLY | A | 204 | 95.936 | 24.686 | 29.495 | 1.00 | 22.27 |
| ATOM | 1955 | O | GLY | A | 204 | 94.900 | 24.098 | 29.206 | 1.00 | 24.27 |
| ATOM | 1956 | N | ILE | A | 205 | 96.909 | 24.893 | 28.619 | 1.00 | 22.81 |
| ATOM | 1958 | CA | ILE | A | 205 | 96.817 | 24.402 | 27.250 | 1.00 | 21.62 |
| ATOM | 1959 | CB | ILE | A | 205 | 96.868 | 25.542 | 26.222 | 1.00 | 17.56 |
| ATOM | 1960 | CG2 | ILE | A | 205 | 96.458 | 25.020 | 24.870 | 1.00 | 17.14 |
| ATOM | 1961 | CG1 | ILE | A | 205 | 95.928 | 26.672 | 26.627 | 1.00 | 16.69 |
| ATOM | 1962 | CD1 | ILE | A | 205 | 96.021 | 27.878 | 25.753 | 1.00 | 10.87 |
| ATOM | 1963 | C | ILE | A | 205 | 98.010 | 23.492 | 27.008 | 1.00 | 24.34 |
| ATOM | 1964 | O | ILE | A | 205 | 99.120 | 23.784 | 27.452 | 1.00 | 26.93 |
| ATOM | 1965 | N | ASN | A | 206 | 97.770 | 22.374 | 26.333 | 1.00 | 24.44 |
| ATOM | 1967 | CA | ASN | A | 206 | 98.817 | 21.414 | 26.039 | 1.00 | 20.90 |
| ATOM | 1968 | CB | ASN | A | 206 | 98.280 | 19.989 | 26.217 | 1.00 | 21.75 |
| ATOM | 1969 | CG | ASN | A | 206 | 99.359 | 18.921 | 26.099 | 1.00 | 22.44 |
| ATOM | 1970 | OD1 | ASN | A | 206 | 99.077 | 17.727 | 26.218 | 1.00 | 26.27 |
| ATOM | 1971 | ND2 | ASN | A | 206 | 100.593 | 19.337 | 25.878 | 1.00 | 24.68 |
| ATOM | 1974 | C | ASN | A | 206 | 99.285 | 21.629 | 24.614 | 1.00 | 22.48 |
| ATOM | 1975 | O | ASN | A | 206 | 98.579 | 21.296 | 23.664 | 1.00 | 23.50 |
| ATOM | 1976 | N | TYR | A | 207 | 100.458 | 22.239 | 24.475 | 1.00 | 23.09 |
| ATOM | 1978 | CA | TYR | A | 207 | 101.065 | 22.517 | 23.179 | 1.00 | 21.41 |
| ATOM | 1979 | CB | TYR | A | 207 | 101.647 | 23.927 | 23.165 | 1.00 | 17.69 |
| ATOM | 1980 | CG | TYR | A | 207 | 100.625 | 25.008 | 22.986 | 1.00 | 16.56 |
| ATOM | 1981 | CD1 | TYR | A | 207 | 99.703 | 24.943 | 21.961 | 1.00 | 19.32 |
| ATOM | 1982 | CE1 | TYR | A | 207 | 98.795 | 25.963 | 21.757 | 1.00 | 20.24 |
| ATOM | 1983 | CD2 | TYR | A | 207 | 100.611 | 26.122 | 23.810 | 1.00 | 18.10 |
| ATOM | 1984 | CE2 | TYR | A | 207 | 99.711 | 27.144 | 23.613 | 1.00 | 18.49 |
| ATOM | 1985 | CZ | TYR | A | 207 | 98.809 | 27.060 | 22.581 | 1.00 | 18.16 |
| ATOM | 1986 | OH | TYR | A | 207 | 97.939 | 28.089 | 22.332 | 1.00 | 21.72 |
| ATOM | 1988 | C | TYR | A | 207 | 102.181 | 21.532 | 22.868 | 1.00 | 24.14 |
| ATOM | 1989 | O | TYR | A | 207 | 103.111 | 21.851 | 22.134 | 1.00 | 25.16 |
| ATOM | 1990 | N | GLY | A | 208 | 102.083 | 20.328 | 23.418 | 1.00 | 28.50 |
| ATOM | 1992 | CA | GLY | A | 208 | 103.112 | 19.330 | 23.194 | 1.00 | 29.80 |
| ATOM | 1993 | C | GLY | A | 208 | 103.130 | 18.752 | 21.795 | 1.00 | 32.56 |
| ATOM | 1994 | O | GLY | A | 208 | 102.067 | 18.467 | 21.231 | 1.00 | 33.45 |
| ATOM | 1995 | N | LEU | A | 209 | 104.332 | 18.598 | 21.235 | 1.00 | 33.15 |
| ATOM | 1997 | CA | LEU | A | 209 | 104.512 | 18.038 | 19.899 | 1.00 | 31.79 |
| ATOM | 1998 | CB | LEU | A | 209 | 105.985 | 18.081 | 19.484 | 1.00 | 29.49 |
| ATOM | 1999 | CG | LEU | A | 209 | 106.491 | 19.337 | 18.773 | 1.00 | 28.06 |
| ATOM | 2000 | CD1 | LEU | A | 209 | 106.302 | 20.558 | 19.641 | 1.00 | 24.35 |
| ATOM | 2001 | CD2 | LEU | A | 209 | 107.953 | 19.158 | 18.416 | 1.00 | 24.06 |
| ATOM | 2002 | C | LEU | A | 209 | 104.052 | 16.599 | 19.941 | 1.00 | 31.52 |
| ATOM | 2003 | O | LEU | A | 209 | 104.416 | 15.863 | 20.854 | 1.00 | 33.82 |
| ATOM | 2004 | N | VAL | A | 210 | 103.254 | 16.199 | 18.961 | 1.00 | 29.87 |
| ATOM | 2006 | CA | VAL | A | 210 | 102.752 | 14.836 | 18.915 | 1.00 | 29.99 |
| ATOM | 2007 | CB | VAL | A | 210 | 101.226 | 14.817 | 18.732 | 1.00 | 30.37 |
| ATOM | 2008 | CG1 | VAL | A | 210 | 100.559 | 15.363 | 19.972 | 1.00 | 31.24 |
| ATOM | 2009 | CG2 | VAL | A | 210 | 100.824 | 15.642 | 17.522 | 1.00 | 30.83 |
| ATOM | 2010 | C | VAL | A | 210 | 103.430 | 13.992 | 17.837 | 1.00 | 30.78 |
| ATOM | 2011 | O | VAL | A | 210 | 103.038 | 12.854 | 17.578 | 1.00 | 32.27 |
| ATOM | 2012 | N | ALA | A | 211 | 104.460 | 14.552 | 17.221 | 1.00 | 30.97 |
| ATOM | 2014 | CA | ALA | A | 211 | 105.199 | 13.857 | 16.178 | 1.00 | 32.26 |
| ATOM | 2015 | CB | ALA | A | 211 | 104.324 | 13.661 | 14.941 | 1.00 | 34.28 |
| ATOM | 2016 | C | ALA | A | 211 | 106.417 | 14.692 | 15.839 | 1.00 | 31.64 |
| ATOM | 2017 | O | ALA | A | 211 | 106.463 | 15.884 | 16.145 | 1.00 | 32.86 |
| ATOM | 2018 | N | PRO | A | 212 | 107.437 | 14.077 | 15.232 | 1.00 | 30.38 |
| ATOM | 2019 | CD | PRO | A | 212 | 107.550 | 12.667 | 14.820 | 1.00 | 28.87 |
| ATOM | 2020 | CA | PRO | A | 212 | 108.642 | 14.824 | 14.876 | 1.00 | 30.59 |
| ATOM | 2021 | CB | PRO | A | 212 | 109.576 | 13.724 | 14.384 | 1.00 | 30.90 |
| ATOM | 2022 | CG | PRO | A | 212 | 108.628 | 12.731 | 13.785 | 1.00 | 31.64 |
| ATOM | 2023 | C | PRO | A | 212 | 108.345 | 15.824 | 13.774 | 1.00 | 31.31 |
| ATOM | 2024 | O | PRO | A | 212 | 107.483 | 15.574 | 12.938 | 1.00 | 33.01 |
| ATOM | 2025 | N | PRO | A | 213 | 109.005 | 16.996 | 13.802 | 1.00 | 30.25 |
| ATOM | 2026 | CD | PRO | A | 213 | 109.882 | 17.457 | 14.892 | 1.00 | 28.21 |
| ATOM | 2027 | CA | PRO | A | 213 | 108.832 | 18.056 | 12.803 | 1.00 | 29.74 |
| ATOM | 2028 | CB | PRO | A | 213 | 109.889 | 19.076 | 13.220 | 1.00 | 29.54 |
| ATOM | 2029 | CG | PRO | A | 213 | 109.896 | 18.958 | 14.690 | 1.00 | 26.77 |
| ATOM | 2030 | C | PRO | A | 213 | 109.115 | 17.526 | 11.398 | 1.00 | 30.72 |
| ATOM | 2031 | O | PRO | A | 213 | 110.187 | 16.975 | 11.140 | 1.00 | 33.55 |
| ATOM | 2032 | N | ALA | A | 214 | 108.157 | 17.700 | 10.495 | 1.00 | 30.16 |
| ATOM | 2034 | CA | ALA | A | 214 | 108.296 | 17.231 | 9.123 | 1.00 | 29.31 |
| ATOM | 2035 | CB | ALA | A | 214 | 106.964 | 16.701 | 8.609 | 1.00 | 27.36 |
| ATOM | 2036 | C | ALA | A | 214 | 108.771 | 18.336 | 8.212 | 1.00 | 30.52 |
| ATOM | 2037 | O | ALA | A | 214 | 108.229 | 19.440 | 8.235 | 1.00 | 32.72 |
| ATOM | 2038 | N | THR | A | 215 | 109.781 | 18.052 | 7.405 | 1.00 | 30.13 |
| ATOM | 2040 | CA | THR | A | 215 | 110.268 | 19.047 | 6.472 | 1.00 | 31.47 |
| ATOM | 2041 | CB | THR | A | 215 | 111.805 | 19.044 | 6.366 | 1.00 | 32.14 |
| ATOM | 2042 | OG1 | THR | A | 215 | 112.381 | 19.228 | 7.665 | 1.00 | 36.33 |
| ATOM | 2044 | CG2 | THR | A | 215 | 112.268 | 20.177 | 5.474 | 1.00 | 33.34 |
| ATOM | 2045 | C | THR | A | 215 | 109.656 | 18.698 | 5.127 | 1.00 | 32.34 |
| ATOM | 2046 | O | THR | A | 215 | 109.642 | 17.533 | 4.729 | 1.00 | 32.18 |
| ATOM | 2047 | N | THR | A | 216 | 109.103 | 19.692 | 4.451 | 1.00 | 34.34 |
| ATOM | 2049 | CA | THR | A | 216 | 108.489 | 19.463 | 3.154 | 1.00 | 36.27 |
| ATOM | 2050 | CB | THR | A | 216 | 106.996 | 19.862 | 3.167 | 1.00 | 36.83 |
| ATOM | 2051 | OG1 | THR | A | 216 | 106.872 | 21.255 | 3.477 | 1.00 | 37.89 |
| ATOM | 2053 | CG2 | THR | A | 216 | 106.241 | 19.055 | 4.216 | 1.00 | 36.73 |
| ATOM | 2054 | C | THR | A | 216 | 109.247 | 20.254 | 2.092 | 1.00 | 37.93 |
| ATOM | 2055 | O | THR | A | 216 | 110.390 | 20.642 | 2.307 | 1.00 | 36.96 |
| ATOM | 2056 | N | ALA | A | 217 | 108.610 | 20.483 | 0.948 | 1.00 | 40.73 |
| ATOM | 2058 | CA | ALA | A | 217 | 109.228 | 21.215 | −0.151 | 1.00 | 41.25 |
| ATOM | 2059 | CB | ALA | A | 217 | 108.452 | 20.967 | −1.437 | 1.00 | 42.73 |
| ATOM | 2060 | C | ALA | A | 217 | 109.302 | 22.704 | 0.125 | 1.00 | 40.38 |
| ATOM | 2061 | O | ALA | A | 217 | 110.218 | 23.379 | −0.337 | 1.00 | 40.36 |
| ATOM | 2062 | N | GLU | A | 218 | 108.319 | 23.215 | 0.860 | 1.00 | 41.20 |
| ATOM | 2064 | CA | GLU | A | 218 | 108.271 | 24.633 | 1.170 | 1.00 | 40.88 |
| ATOM | 2065 | CB | GLU | A | 218 | 107.443 | 25.372 | 0.113 | 1.00 | 46.00 |
| ATOM | 2066 | CG | GLU | A | 218 | 108.304 | 26.058 | −0.947 | 1.00 | 53.45 |
| ATOM | 2067 | CD | GLU | A | 218 | 107.610 | 26.213 | −2.286 | 1.00 | 57.50 |
| ATOM | 2068 | OE1 | GLU | A | 218 | 106.543 | 25.588 | −2.487 | 1.00 | 60.01 |
| ATOM | 2069 | OE2 | GLU | A | 218 | 108.149 | 26.945 | −3.146 | 1.00 | 59.32 |
| ATOM | 2070 | C | GLU | A | 218 | 107.832 | 25.030 | 2.580 | 1.00 | 39.13 |
| ATOM | 2071 | O | GLU | A | 218 | 107.635 | 26.216 | 2.839 | 1.00 | 40.54 |
| ATOM | 2072 | N | THR | A | 219 | 107.668 | 24.066 | 3.488 | 1.00 | 37.20 |
| ATOM | 2074 | CA | THR | A | 219 | 107.295 | 24.388 | 4.875 | 1.00 | 35.55 |
| ATOM | 2075 | CB | THR | A | 219 | 105.762 | 24.464 | 5.103 | 1.00 | 32.68 |
| ATOM | 2076 | OG1 | THR | A | 219 | 105.144 | 23.249 | 4.674 | 1.00 | 35.48 |
| ATOM | 2078 | CG2 | THR | A | 219 | 105.151 | 25.641 | 4.387 | 1.00 | 33.81 |
| ATOM | 2079 | C | THR | A | 219 | 107.828 | 23.400 | 5.906 | 1.00 | 34.31 |
| ATOM | 2080 | O | THR | A | 219 | 108.013 | 22.217 | 5.614 | 1.00 | 34.57 |
| ATOM | 2081 | N | LEU | A | 220 | 108.087 | 23.903 | 7.109 | 1.00 | 31.67 |
| ATOM | 2083 | CA | LEU | A | 220 | 108.533 | 23.079 | 8.219 | 1.00 | 29.70 |
| ATOM | 2084 | CB | LEU | A | 220 | 109.587 | 23.834 | 9.033 | 1.00 | 29.62 |
| ATOM | 2085 | CG | LEU | A | 220 | 110.345 | 23.149 | 10.177 | 1.00 | 31.10 |
| ATOM | 2086 | CD1 | LEU | A | 220 | 109.511 | 23.047 | 11.435 | 1.00 | 30.08 |
| ATOM | 2087 | CD2 | LEU | A | 220 | 110.794 | 21.791 | 9.727 | 1.00 | 30.78 |
| ATOM | 2088 | C | LEU | A | 220 | 107.236 | 22.902 | 9.017 | 1.00 | 29.78 |
| ATOM | 2089 | O | LEU | A | 220 | 106.693 | 23.878 | 9.545 | 1.00 | 30.49 |
| ATOM | 2090 | N | ASP | A | 221 | 106.698 | 21.685 | 9.032 | 1.00 | 29.15 |
| ATOM | 2092 | CA | ASP | A | 221 | 105.441 | 21.397 | 9.728 | 1.00 | 30.48 |
| ATOM | 2093 | CB | ASP | A | 221 | 104.603 | 20.394 | 8.924 | 1.00 | 34.57 |
| ATOM | 2094 | CG | ASP | A | 221 | 104.178 | 20.924 | 7.557 | 1.00 | 37.91 |
| ATOM | 2095 | OD1 | ASP | A | 221 | 104.480 | 22.090 | 7.222 | 1.00 | 37.84 |
| ATOM | 2096 | OD2 | ASP | A | 221 | 103.534 | 20.158 | 6.811 | 1.00 | 42.30 |
| ATOM | 2097 | C | ASP | A | 221 | 105.607 | 20.865 | 11.143 | 1.00 | 30.64 |
| ATOM | 2098 | O | ASP | A | 221 | 106.274 | 19.852 | 11.350 | 1.00 | 31.22 |
| ATOM | 2099 | N | VAL | A | 222 | 104.950 | 21.519 | 12.100 | 1.00 | 29.81 |
| ATOM | 2101 | CA | VAL | A | 222 | 104.999 | 21.131 | 13.512 | 1.00 | 29.49 |
| ATOM | 2102 | CB | VAL | A | 222 | 105.588 | 22.261 | 14.389 | 1.00 | 29.52 |
| ATOM | 2103 | CG1 | VAL | A | 222 | 105.617 | 21.831 | 15.845 | 1.00 | 28.17 |
| ATOM | 2104 | CG2 | VAL | A | 222 | 106.988 | 22.632 | 13.914 | 1.00 | 26.55 |
| ATOM | 2105 | C | VAL | A | 222 | 103.586 | 20.791 | 14.016 | 1.00 | 29.65 |
| ATOM | 2106 | O | VAL | A | 222 | 102.686 | 21.620 | 13.958 | 1.00 | 31.27 |
| ATOM | 2107 | N | GLN | A | 223 | 103.400 | 19.560 | 14.485 | 1.00 | 29.35 |
| ATOM | 2109 | CA | GLN | A | 223 | 102.114 | 19.081 | 14.978 | 1.00 | 25.88 |
| ATOM | 2110 | CB | GLN | A | 223 | 101.870 | 17.649 | 14.488 | 1.00 | 29.16 |
| ATOM | 2111 | CG | GLN | A | 223 | 101.731 | 17.505 | 12.969 | 1.00 | 30.27 |
| ATOM | 2112 | CD | GLN | A | 223 | 101.704 | 16.052 | 12.510 | 1.00 | 32.10 |
| ATOM | 2113 | OE1 | GLN | A | 223 | 100.832 | 15.275 | 12.897 | 1.00 | 31.46 |
| ATOM | 2114 | NE2 | GLN | A | 223 | 102.668 | 15.681 | 11.686 | 1.00 | 37.75 |
| ATOM | 2117 | C | GLN | A | 223 | 102.081 | 19.116 | 16.494 | 1.00 | 24.57 |
| ATOM | 2118 | O | GLN | A | 223 | 102.910 | 18.490 | 17.147 | 1.00 | 23.51 |
| ATOM | 2119 | N | MET | A | 224 | 101.112 | 19.840 | 17.042 | 1.00 | 22.94 |
| ATOM | 2121 | CA | MET | A | 224 | 100.959 | 19.984 | 18.480 | 1.00 | 22.16 |
| ATOM | 2122 | CB | MET | A | 224 | 101.064 | 21.459 | 18.866 | 1.00 | 23.89 |
| ATOM | 2123 | CG | MET | A | 224 | 102.353 | 22.116 | 18.423 | 1.00 | 22.07 |
| ATOM | 2124 | SD | MET | A | 224 | 102.393 | 23.853 | 18.797 | 1.00 | 28.37 |
| ATOM | 2125 | CE | MET | A | 224 | 104.122 | 24.130 | 18.771 | 1.00 | 25.60 |
| ATOM | 2126 | C | MET | A | 224 | 99.620 | 19.408 | 18.927 | 1.00 | 23.31 |
| ATOM | 2127 | O | MET | A | 224 | 98.702 | 19.294 | 18.124 | 1.00 | 27.48 |
| ATOM | 2128 | N | LYS | A | 225 | 99.519 | 19.024 | 20.197 | 1.00 | 25.28 |
| ATOM | 2130 | CA | LYS | A | 225 | 98.292 | 18.440 | 20.735 | 1.00 | 27.30 |
| ATOM | 2131 | CB | LYS | A | 225 | 98.522 | 17.889 | 22.140 | 1.00 | 28.45 |
| ATOM | 2132 | CG | LYS | A | 225 | 97.613 | 16.724 | 22.452 | 1.00 | 30.88 |
| ATOM | 2133 | CD | LYS | A | 225 | 97.912 | 16.108 | 23.793 | 1.00 | 33.18 |
| ATOM | 2134 | CE | LYS | A | 225 | 97.153 | 14.802 | 23.938 | 1.00 | 38.68 |
| ATOM | 2135 | NZ | LYS | A | 225 | 95.741 | 14.928 | 23.460 | 1.00 | 43.39 |
| ATOM | 2139 | C | LYS | A | 225 | 97.135 | 19.434 | 20.726 | 1.00 | 27.30 |
| ATOM | 2140 | O | LYS | A | 225 | 96.052 | 19.123 | 20.238 | 1.00 | 29.51 |
| ATOM | 2141 | N | GLY | A | 226 | 97.364 | 20.617 | 21.286 | 1.00 | 26.18 |
| ATOM | 2143 | CA | GLY | A | 226 | 96.360 | 21.665 | 21.285 | 1.00 | 21.95 |
| ATOM | 2144 | C | GLY | A | 226 | 95.015 | 21.379 | 21.903 | 1.00 | 23.48 |
| ATOM | 2145 | O | GLY | A | 226 | 93.983 | 21.420 | 21.238 | 1.00 | 23.78 |
| ATOM | 2146 | N | GLU | A | 227 | 95.021 | 21.131 | 23.201 | 1.00 | 26.02 |
| ATOM | 2148 | CA | GLU | A | 227 | 93.792 | 20.882 | 23.915 | 1.00 | 26.24 |
| ATOM | 2149 | CB | GLU | A | 227 | 93.547 | 19.384 | 24.068 | 1.00 | 29.17 |
| ATOM | 2150 | CG | GLU | A | 227 | 94.467 | 18.697 | 25.052 | 1.00 | 25.81 |
| ATOM | 2151 | CD | GLU | A | 227 | 93.967 | 17.330 | 25.456 | 1.00 | 29.15 |
| ATOM | 2152 | OE1 | GLU | A | 227 | 93.343 | 16.654 | 24.614 | 1.00 | 33.43 |
| ATOM | 2153 | OE2 | GLU | A | 227 | 94.199 | 16.928 | 26.615 | 1.00 | 31.54 |
| ATOM | 2154 | C | GLU | A | 227 | 93.937 | 21.514 | 25.283 | 1.00 | 27.06 |
| ATOM | 2155 | O | GLU | A | 227 | 95.006 | 21.447 | 25.892 | 1.00 | 26.79 |
| ATOM | 2156 | N | PHE | A | 228 | 92.904 | 22.219 | 25.722 | 1.00 | 26.37 |
| ATOM | 2158 | CA | PHE | A | 228 | 92.942 | 22.816 | 27.034 | 1.00 | 24.07 |
| ATOM | 2159 | CB | PHE | A | 228 | 91.785 | 23.792 | 27.242 | 1.00 | 24.55 |
| ATOM | 2160 | CG | PHE | A | 228 | 91.987 | 25.134 | 26.592 | 1.00 | 23.72 |
| ATOM | 2161 | CD1 | PHE | A | 228 | 92.066 | 25.251 | 25.207 | 1.00 | 21.59 |
| ATOM | 2162 | CD2 | PHE | A | 228 | 92.045 | 26.287 | 27.366 | 1.00 | 21.39 |
| ATOM | 2163 | CE1 | PHE | A | 228 | 92.193 | 26.498 | 24.607 | 1.00 | 21.74 |
| ATOM | 2164 | CE2 | PHE | A | 228 | 92.172 | 27.531 | 26.779 | 1.00 | 21.24 |
| ATOM | 2165 | CZ | PHE | A | 228 | 92.246 | 27.640 | 25.395 | 1.00 | 24.55 |
| ATOM | 2166 | C | PHE | A | 228 | 92.779 | 21.630 | 27.949 | 1.00 | 25.56 |
| ATOM | 2167 | O | PHE | A | 228 | 92.069 | 20.680 | 27.624 | 1.00 | 26.15 |
| ATOM | 2168 | N | TYR | A | 229 | 93.479 | 21.655 | 29.067 | 1.00 | 29.13 |
| ATOM | 2170 | CA | TYR | A | 229 | 93.400 | 20.573 | 30.025 | 1.00 | 34.87 |
| ATOM | 2171 | CB | TYR | A | 229 | 94.735 | 19.820 | 30.084 | 1.00 | 32.93 |
| ATOM | 2172 | CG | TYR | A | 229 | 95.870 | 20.654 | 30.639 | 1.00 | 33.77 |
| ATOM | 2173 | CD1 | TYR | A | 229 | 96.034 | 20.816 | 32.015 | 1.00 | 34.08 |
| ATOM | 2174 | CE1 | TYR | A | 229 | 97.021 | 21.637 | 32.530 | 1.00 | 33.96 |
| ATOM | 2175 | CD2 | TYR | A | 229 | 96.739 | 21.332 | 29.793 | 1.00 | 34.25 |
| ATOM | 2176 | CE2 | TYR | A | 229 | 97.735 | 22.152 | 30.302 | 1.00 | 34.53 |
| ATOM | 2177 | CZ | TYR | A | 229 | 97.863 | 22.302 | 31.670 | 1.00 | 33.54 |
| ATOM | 2178 | OH | TYR | A | 229 | 98.819 | 23.138 | 32.185 | 1.00 | 38.50 |
| ATOM | 2180 | C | TYR | A | 229 | 93.097 | 21.196 | 31.380 | 1.00 | 39.53 |
| ATOM | 2181 | O | TYR | A | 229 | 93.192 | 22.414 | 31.545 | 1.00 | 39.55 |
| ATOM | 2182 | N | SER | A | 230 | 92.738 | 20.355 | 32.341 | 1.00 | 46.28 |
| ATOM | 2184 | CA | SER | A | 230 | 92.453 | 20.796 | 33.699 | 1.00 | 53.45 |
| ATOM | 2185 | CB | SER | A | 230 | 90.998 | 20.491 | 34.071 | 1.00 | 54.47 |
| ATOM | 2186 | OG | SER | A | 230 | 90.720 | 20.820 | 35.424 | 1.00 | 56.07 |
| ATOM | 2188 | C | SER | A | 230 | 93.396 | 19.996 | 34.582 | 1.00 | 58.48 |
| ATOM | 2189 | O | SER | A | 230 | 93.659 | 18.826 | 34.301 | 1.00 | 58.09 |
| ATOM | 2190 | N | GLU | A | 231 | 93.946 | 20.632 | 35.611 | 1.00 | 66.92 |
| ATOM | 2192 | CA | GLU | A | 231 | 94.860 | 19.940 | 36.517 | 1.00 | 74.85 |
| ATOM | 2193 | CB | GLU | A | 231 | 95.976 | 20.878 | 36.995 | 1.00 | 77.02 |
| ATOM | 2194 | CG | GLU | A | 231 | 97.377 | 20.450 | 36.548 | 1.00 | 81.62 |
| ATOM | 2195 | CD | GLU | A | 231 | 98.476 | 21.372 | 37.054 | 1.00 | 84.67 |
| ATOM | 2196 | OE1 | GLU | A | 231 | 98.402 | 22.592 | 36.790 | 1.00 | 86.64 |
| ATOM | 2197 | OE2 | GLU | A | 231 | 99.421 | 20.876 | 37.709 | 1.00 | 85.89 |
| ATOM | 2198 | C | GLU | A | 231 | 94.119 | 19.328 | 37.706 | 1.00 | 78.17 |
| ATOM | 2199 | O | GLU | A | 231 | 94.602 | 18.379 | 38.323 | 1.00 | 79.46 |
| ATOM | 2200 | N | ALA | A | 232 | 92.934 | 19.855 | 38.003 | 1.00 | 81.24 |
| ATOM | 2202 | CA | ALA | A | 232 | 92.131 | 19.363 | 39.118 | 1.00 | 84.16 |
| ATOM | 2203 | CB | ALA | A | 232 | 91.111 | 20.417 | 39.544 | 1.00 | 84.14 |
| ATOM | 2204 | C | ALA | A | 232 | 91.426 | 18.053 | 38.777 | 1.00 | 86.04 |
| ATOM | 2205 | O | ALA | A | 232 | 91.078 | 17.279 | 39.671 | 1.00 | 87.55 |
| ATOM | 2206 | N | ALA | A | 233 | 91.203 | 17.819 | 37.487 | 1.00 | 86.79 |
| ATOM | 2208 | CA | ALA | A | 233 | 90.534 | 16.608 | 37.023 | 1.00 | 88.81 |
| ATOM | 2209 | CB | ALA | A | 233 | 89.052 | 16.659 | 37.381 | 1.00 | 89.87 |
| ATOM | 2210 | C | ALA | A | 233 | 90.702 | 16.464 | 35.516 | 1.00 | 90.03 |
| ATOM | 2211 | O | ALA | A | 233 | 90.288 | 17.341 | 34.756 | 1.00 | 90.77 |
| ATOM | 2212 | N | ALA | A | 234 | 91.309 | 15.361 | 35.090 | 1.00 | 91.16 |
| ATOM | 2214 | CA | ALA | A | 234 | 91.536 | 15.106 | 33.670 | 1.00 | 91.66 |
| ATOM | 2215 | CB | ALA | A | 234 | 92.810 | 14.288 | 33.470 | 1.00 | 92.37 |
| ATOM | 2216 | C | ALA | A | 234 | 90.354 | 14.394 | 33.034 | 1.00 | 91.31 |
| ATOM | 2217 | O | ALA | A | 234 | 89.874 | 13.384 | 33.550 | 1.00 | 91.09 |
| ATOM | 2218 | N | ALA | A | 235 | 89.894 | 14.926 | 31.908 | 1.00 | 91.34 |
| ATOM | 2220 | CA | ALA | A | 235 | 88.774 | 14.346 | 31.180 | 1.00 | 90.88 |
| ATOM | 2221 | CB | ALA | A | 235 | 87.457 | 14.975 | 31.627 | 1.00 | 91.57 |
| ATOM | 2222 | C | ALA | A | 235 | 88.993 | 14.572 | 29.691 | 1.00 | 89.83 |
| ATOM | 2223 | O | ALA | A | 235 | 89.205 | 15.706 | 29.247 | 1.00 | 89.77 |
| ATOM | 2224 | N | PRO | A | 236 | 89.032 | 13.480 | 28.912 | 1.00 | 88.56 |
| ATOM | 2225 | CD | PRO | A | 236 | 88.962 | 12.083 | 29.376 | 1.00 | 88.69 |
| ATOM | 2226 | CA | PRO | A | 236 | 89.232 | 13.539 | 27.463 | 1.00 | 85.91 |
| ATOM | 2227 | CB | PRO | A | 236 | 89.511 | 12.080 | 27.104 | 1.00 | 86.56 |
| ATOM | 2228 | CG | PRO | A | 236 | 88.680 | 11.334 | 28.099 | 1.00 | 88.16 |
| ATOM | 2229 | C | PRO | A | 236 | 87.997 | 14.085 | 26.738 | 1.00 | 82.58 |
| ATOM | 2230 | O | PRO | A | 236 | 86.861 | 13.877 | 27.172 | 1.00 | 81.75 |
| ATOM | 2231 | N | PRO | A | 237 | 88.217 | 14.829 | 25.644 | 1.00 | 79.77 |
| ATOM | 2232 | CD | PRO | A | 237 | 89.551 | 15.264 | 25.202 | 1.00 | 80.44 |
| ATOM | 2233 | CA | PRO | A | 237 | 87.178 | 15.444 | 24.811 | 1.00 | 77.34 |
| ATOM | 2234 | CB | PRO | A | 237 | 87.985 | 16.332 | 23.863 | 1.00 | 77.69 |
| ATOM | 2235 | CG | PRO | A | 237 | 89.249 | 16.604 | 24.617 | 1.00 | 79.85 |
| ATOM | 2236 | C | PRO | A | 237 | 86.366 | 14.426 | 24.015 | 1.00 | 75.23 |
| ATOM | 2237 | O | PRO | A | 237 | 86.919 | 13.462 | 23.480 | 1.00 | 75.90 |
| ATOM | 2238 | N | PRO | A | 238 | 85.042 | 14.634 | 23.918 | 1.00 | 72.71 |
| ATOM | 2239 | CD | PRO | A | 238 | 84.282 | 15.606 | 24.724 | 1.00 | 72.84 |
| ATOM | 2240 | CA | PRO | A | 238 | 84.131 | 13.750 | 23.182 | 1.00 | 70.12 |
| ATOM | 2241 | CB | PRO | A | 238 | 82.774 | 14.086 | 23.793 | 1.00 | 71.89 |
| ATOM | 2242 | CG | PRO | A | 238 | 82.907 | 15.535 | 24.109 | 1.00 | 72.51 |
| ATOM | 2243 | C | PRO | A | 238 | 84.146 | 14.004 | 21.672 | 1.00 | 67.49 |
| ATOM | 2244 | O | PRO | A | 238 | 83.099 | 14.016 | 21.017 | 1.00 | 66.70 |
| ATOM | 2245 | N | PHE | A | 239 | 85.340 | 14.216 | 21.130 | 1.00 | 64.79 |
| ATOM | 2247 | CA | PHE | A | 239 | 85.521 | 14.472 | 19.706 | 1.00 | 61.01 |
| ATOM | 2248 | CB | PHE | A | 239 | 84.959 | 15.849 | 19.319 | 1.00 | 60.29 |
| ATOM | 2249 | CG | PHE | A | 239 | 85.172 | 16.923 | 20.362 | 1.00 | 57.87 |
| ATOM | 2250 | CD1 | PHE | A | 239 | 86.307 | 17.721 | 20.339 | 1.00 | 56.42 |
| ATOM | 2251 | CD2 | PHE | A | 239 | 84.216 | 17.159 | 21.343 | 1.00 | 55.75 |
| ATOM | 2252 | CE1 | PHE | A | 239 | 86.484 | 18.737 | 21.274 | 1.00 | 53.82 |
| ATOM | 2253 | CE2 | PHE | A | 239 | 84.387 | 18.171 | 22.279 | 1.00 | 53.85 |
| ATOM | 2254 | CZ | PHE | A | 239 | 85.522 | 18.960 | 22.243 | 1.00 | 52.72 |
| ATOM | 2255 | C | PHE | A | 239 | 86.989 | 14.355 | 19.307 | 1.00 | 58.61 |
| ATOM | 2256 | O | PHE | A | 239 | 87.873 | 14.266 | 20.164 | 1.00 | 58.11 |
| ATOM | 2257 | N | ALA | A | 240 | 87.234 | 14.314 | 18.002 | 1.00 | 55.15 |
| ATOM | 2259 | CA | ALA | A | 240 | 88.586 | 14.203 | 17.472 | 1.00 | 51.42 |
| ATOM | 2260 | CB | ALA | A | 240 | 88.716 | 12.936 | 16.631 | 1.00 | 51.91 |
| ATOM | 2261 | C | ALA | A | 240 | 88.884 | 15.441 | 16.629 | 1.00 | 49.88 |
| ATOM | 2262 | O | ALA | A | 240 | 87.972 | 16.034 | 16.048 | 1.00 | 48.56 |
| ATOM | 2263 | N | PRO | A | 241 | 90.155 | 15.885 | 16.604 | 1.00 | 48.54 |
| ATOM | 2264 | CD | PRO | A | 241 | 91.270 | 15.398 | 17.436 | 1.00 | 47.09 |
| ATOM | 2265 | CA | PRO | A | 241 | 90.567 | 17.064 | 15.830 | 1.00 | 46.53 |
| ATOM | 2266 | CB | PRO | A | 241 | 92.043 | 17.218 | 16.205 | 1.00 | 44.66 |
| ATOM | 2267 | CG | PRO | A | 241 | 92.113 | 16.633 | 17.574 | 1.00 | 45.79 |
| ATOM | 2268 | C | PRO | A | 241 | 90.412 | 16.864 | 14.322 | 1.00 | 46.25 |
| ATOM | 2269 | O | PRO | A | 241 | 90.536 | 15.743 | 13.819 | 1.00 | 47.76 |
| ATOM | 2270 | N | PRO | A | 242 | 90.118 | 17.947 | 13.582 | 1.00 | 45.32 |
| ATOM | 2271 | CD | PRO | A | 242 | 89.818 | 19.314 | 14.050 | 1.00 | 44.47 |
| ATOM | 2272 | CA | PRO | A | 242 | 89.959 | 17.844 | 12.130 | 1.00 | 44.33 |
| ATOM | 2273 | CB | PRO | A | 242 | 89.187 | 19.114 | 11.792 | 1.00 | 43.70 |
| ATOM | 2274 | CG | PRO | A | 242 | 89.769 | 20.107 | 12.755 | 1.00 | 43.85 |
| ATOM | 2275 | C | PRO | A | 242 | 91.325 | 17.835 | 11.442 | 1.00 | 45.10 |
| ATOM | 2276 | O | PRO | A | 242 | 92.331 | 18.261 | 12.019 | 1.00 | 44.03 |
| ATOM | 2277 | N | VAL | A | 243 | 91.370 | 17.302 | 10.228 | 1.00 | 45.79 |
| ATOM | 2279 | CA | VAL | A | 243 | 92.615 | 17.279 | 9.485 | 1.00 | 47.91 |
| ATOM | 2280 | CB | VAL | A | 243 | 92.613 | 16.226 | 8.347 | 1.00 | 48.72 |
| ATOM | 2281 | CG1 | VAL | A | 243 | 92.629 | 14.827 | 8.934 | 1.00 | 50.59 |
| ATOM | 2282 | CG2 | VAL | A | 243 | 91.403 | 16.401 | 7.446 | 1.00 | 51.20 |
| ATOM | 2283 | C | VAL | A | 243 | 92.753 | 18.679 | 8.926 | 1.00 | 48.35 |
| ATOM | 2284 | O | VAL | A | 243 | 91.939 | 19.122 | 8.116 | 1.00 | 48.79 |
| ATOM | 2285 | N | MET | A | 244 | 93.731 | 19.412 | 9.434 | 1.00 | 48.68 |
| ATOM | 2287 | CA | MET | A | 244 | 93.937 | 20.771 | 8.987 | 1.00 | 46.91 |
| ATOM | 2288 | CB | MET | A | 244 | 94.406 | 21.638 | 10.147 | 1.00 | 42.52 |
| ATOM | 2289 | CG | MET | A | 244 | 93.369 | 21.770 | 11.233 | 1.00 | 30.61 |
| ATOM | 2290 | SD | MET | A | 244 | 93.963 | 22.834 | 12.505 | 1.00 | 30.25 |
| ATOM | 2291 | CE | MET | A | 244 | 94.948 | 21.710 | 13.447 | 1.00 | 25.62 |
| ATOM | 2292 | C | MET | A | 244 | 94.884 | 20.877 | 7.811 | 1.00 | 50.14 |
| ATOM | 2293 | O | MET | A | 244 | 96.102 | 20.935 | 7.983 | 1.00 | 50.38 |
| ATOM | 2294 | N | GLU | A | 245 | 94.302 | 20.867 | 6.615 | 1.00 | 53.76 |
| ATOM | 2296 | CA | GLU | A | 245 | 95.046 | 20.985 | 5.367 | 1.00 | 58.60 |
| ATOM | 2297 | CB | GLU | A | 245 | 94.597 | 19.905 | 4.364 | 1.00 | 61.51 |
| ATOM | 2298 | CG | GLU | A | 245 | 93.085 | 19.861 | 4.078 | 1.00 | 67.59 |
| ATOM | 2299 | CD | GLU | A | 245 | 92.692 | 18.811 | 3.041 | 1.00 | 69.79 |
| ATOM | 2300 | OE1 | GLU | A | 245 | 92.943 | 19.024 | 1.827 | 1.00 | 72.17 |
| ATOM | 2301 | OE2 | GLU | A | 245 | 92.104 | 17.778 | 3.439 | 1.00 | 68.95 |
| ATOM | 2302 | C | GLU | A | 245 | 94.817 | 22.382 | 4.794 | 1.00 | 59.59 |
| ATOM | 2303 | O | GLU | A | 245 | 93.765 | 22.664 | 4.220 | 1.00 | 60.79 |
| ATOM | 2304 | N | PHE | A | 246 | 95.773 | 23.278 | 5.010 | 1.00 | 61.44 |
| ATOM | 2306 | CA | PHE | A | 246 | 95.637 | 24.635 | 4.510 | 1.00 | 63.27 |
| ATOM | 2307 | CB | PHE | A | 246 | 95.567 | 25.658 | 5.657 | 1.00 | 60.46 |
| ATOM | 2308 | CG | PHE | A | 246 | 96.689 | 25.567 | 6.663 | 1.00 | 56.43 |
| ATOM | 2309 | CD1 | PHE | A | 246 | 97.850 | 26.319 | 6.502 | 1.00 | 54.28 |
| ATOM | 2310 | CD2 | PHE | A | 246 | 96.532 | 24.825 | 7.834 | 1.00 | 53.72 |
| ATOM | 2311 | CE1 | PHE | A | 246 | 98.829 | 26.342 | 7.497 | 1.00 | 51.24 |
| ATOM | 2312 | CE2 | PHE | A | 246 | 97.506 | 24.841 | 8.834 | 1.00 | 51.21 |
| ATOM | 2313 | CZ | PHE | A | 246 | 98.653 | 25.602 | 8.667 | 1.00 | 48.89 |
| ATOM | 2314 | C | PHE | A | 246 | 96.676 | 25.022 | 3.473 | 1.00 | 67.53 |
| ATOM | 2315 | O | PHE | A | 246 | 97.723 | 24.382 | 3.357 | 1.00 | 68.37 |
| ATOM | 2316 | N | PRO | A | 247 | 96.375 | 26.050 | 2.664 | 1.00 | 71.27 |
| ATOM | 2317 | CD | PRO | A | 247 | 95.153 | 26.874 | 2.673 | 1.00 | 72.37 |
| ATOM | 2318 | CA | PRO | A | 247 | 97.297 | 26.511 | 1.624 | 1.00 | 74.18 |
| ATOM | 2319 | CB | PRO | A | 247 | 96.504 | 27.627 | 0.938 | 1.00 | 74.64 |
| ATOM | 2320 | CG | PRO | A | 247 | 95.626 | 28.147 | 2.038 | 1.00 | 73.71 |
| ATOM | 2321 | C | PRO | A | 247 | 98.620 | 27.016 | 2.180 | 1.00 | 76.26 |
| ATOM | 2322 | O | PRO | A | 247 | 98.661 | 28.002 | 2.920 | 1.00 | 76.91 |
| ATOM | 2323 | N | ALA | A | 248 | 99.691 | 26.299 | 1.858 | 1.00 | 78.37 |
| ATOM | 2325 | CA | ALA | A | 248 | 101.027 | 26.675 | 2.298 | 1.00 | 80.34 |
| ATOM | 2326 | CB | ALA | A | 248 | 102.025 | 25.578 | 1.963 | 1.00 | 80.57 |
| ATOM | 2327 | C | ALA | A | 248 | 101.385 | 27.950 | 1.556 | 1.00 | 81.06 |
| ATOM | 2328 | O | ALA | A | 248 | 101.463 | 27.962 | 0.325 | 1.00 | 81.92 |
| ATOM | 2329 | N | ALA | A | 249 | 101.564 | 29.030 | 2.300 | 1.00 | 80.72 |
| ATOM | 2331 | CA | ALA | A | 249 | 101.902 | 30.303 | 1.694 | 1.00 | 79.65 |
| ATOM | 2332 | CB | ALA | A | 249 | 100.799 | 31.319 | 1.964 | 1.00 | 80.65 |
| ATOM | 2333 | C | ALA | A | 249 | 103.232 | 30.799 | 2.234 | 1.00 | 78.22 |
| ATOM | 2334 | O | ALA | A | 249 | 103.799 | 30.201 | 3.150 | 1.00 | 78.60 |
| ATOM | 2335 | N | ALA | A | 250 | 103.734 | 31.878 | 1.643 | 1.00 | 76.27 |
| ATOM | 2337 | CA | ALA | A | 250 | 104.997 | 32.476 | 2.062 | 1.00 | 74.32 |
| ATOM | 2338 | CB | ALA | A | 250 | 106.093 | 32.174 | 1.042 | 1.00 | 75.66 |
| ATOM | 2339 | C | ALA | A | 250 | 104.840 | 33.985 | 2.249 | 1.00 | 71.97 |
| ATOM | 2340 | O | ALA | A | 250 | 105.815 | 34.732 | 2.160 | 1.00 | 72.98 |
| ATOM | 2341 | N | ASP | A | 251 | 103.606 | 34.422 | 2.496 | 1.00 | 67.46 |
| ATOM | 2343 | CA | ASP | A | 251 | 103.302 | 35.836 | 2.708 | 1.00 | 61.94 |
| ATOM | 2344 | CB | ASP | A | 251 | 101.797 | 36.090 | 2.568 | 1.00 | 66.00 |
| ATOM | 2345 | CG | ASP | A | 251 | 101.277 | 35.785 | 1.177 | 1.00 | 70.21 |
| ATOM | 2346 | OD1 | ASP | A | 251 | 101.467 | 34.647 | 0.695 | 1.00 | 71.96 |
| ATOM | 2347 | OD2 | ASP | A | 251 | 100.668 | 36.687 | 0.566 | 1.00 | 72.84 |
| ATOM | 2348 | C | ASP | A | 251 | 103.760 | 36.265 | 4.101 | 1.00 | 56.10 |
| ATOM | 2349 | O | ASP | A | 251 | 104.203 | 37.396 | 4.298 | 1.00 | 56.97 |
| ATOM | 2350 | N | ARG | A | 252 | 103.611 | 35.364 | 5.067 | 1.00 | 47.53 |
| ATOM | 2352 | CA | ARG | A | 252 | 104.009 | 35.622 | 6.441 | 1.00 | 39.41 |
| ATOM | 2353 | CB | ARG | A | 252 | 102.815 | 35.472 | 7.384 | 1.00 | 39.71 |
| ATOM | 2354 | CG | ARG | A | 252 | 101.668 | 36.426 | 7.129 | 1.00 | 39.66 |
| ATOM | 2355 | CD | ARG | A | 252 | 101.975 | 37.837 | 7.596 | 1.00 | 41.34 |
| ATOM | 2356 | NE | ARG | A | 252 | 100.830 | 38.714 | 7.375 | 1.00 | 42.20 |
| ATOM | 2358 | CZ | ARG | A | 252 | 100.628 | 39.416 | 6.263 | 1.00 | 44.51 |
| ATOM | 2359 | NH1 | ARG | A | 252 | 101.498 | 39.358 | 5.263 | 1.00 | 45.26 |
| ATOM | 2362 | NH2 | ARG | A | 252 | 99.542 | 40.163 | 6.141 | 1.00 | 44.83 |
| ATOM | 2365 | C | ARG | A | 252 | 105.070 | 34.594 | 6.798 | 1.00 | 34.91 |
| ATOM | 2366 | O | ARG | A | 252 | 105.315 | 33.667 | 6.035 | 1.00 | 31.70 |
| ATOM | 2367 | N | MET | A | 253 | 105.662 | 34.730 | 7.976 | 1.00 | 32.83 |
| ATOM | 2369 | CA | MET | A | 253 | 106.697 | 33.809 | 8.417 | 1.00 | 30.84 |
| ATOM | 2370 | CB | MET | A | 253 | 107.605 | 34.499 | 9.441 | 1.00 | 30.27 |
| ATOM | 2371 | CG | MET | A | 253 | 108.192 | 35.807 | 8.925 | 1.00 | 31.26 |
| ATOM | 2372 | SD | MET | A | 253 | 109.366 | 36.637 | 10.009 | 1.00 | 34.11 |
| ATOM | 2373 | CE | MET | A | 253 | 110.335 | 37.492 | 8.834 | 1.00 | 29.80 |
| ATOM | 2374 | C | MET | A | 253 | 106.130 | 32.501 | 8.979 | 1.00 | 31.42 |
| ATOM | 2375 | O | MET | A | 253 | 106.568 | 31.419 | 8.595 | 1.00 | 31.42 |
| ATOM | 2376 | N | VAL | A | 254 | 105.144 | 32.600 | 9.866 | 1.00 | 31.26 |
| ATOM | 2378 | CA | VAL | A | 254 | 104.540 | 31.419 | 10.480 | 1.00 | 32.17 |
| ATOM | 2379 | CB | VAL | A | 254 | 104.781 | 31.376 | 12.015 | 1.00 | 32.83 |
| ATOM | 2380 | CG1 | VAL | A | 254 | 106.243 | 31.143 | 12.324 | 1.00 | 35.48 |
| ATOM | 2381 | CG2 | VAL | A | 254 | 104.316 | 32.672 | 12.658 | 1.00 | 33.43 |
| ATOM | 2382 | C | VAL | A | 254 | 103.043 | 31.378 | 10.262 | 1.00 | 32.00 |
| ATOM | 2383 | O | VAL | A | 254 | 102.412 | 32.410 | 10.059 | 1.00 | 33.43 |
| ATOM | 2384 | N | TYR | A | 255 | 102.478 | 30.181 | 10.340 | 1.00 | 33.42 |
| ATOM | 2386 | CA | TYR | A | 255 | 101.044 | 29.979 | 10.179 | 1.00 | 32.60 |
| ATOM | 2387 | CB | TYR | A | 255 | 100.726 | 29.448 | 8.784 | 1.00 | 32.98 |
| ATOM | 2388 | CG | TYR | A | 255 | 100.746 | 30.482 | 7.694 | 1.00 | 32.36 |
| ATOM | 2389 | CD1 | TYR | A | 255 | 101.913 | 30.756 | 6.987 | 1.00 | 31.99 |
| ATOM | 2390 | CE1 | TYR | A | 255 | 101.921 | 31.674 | 5.947 | 1.00 | 33.10 |
| ATOM | 2391 | CD2 | TYR | A | 255 | 99.587 | 31.154 | 7.335 | 1.00 | 31.03 |
| ATOM | 2392 | CE2 | TYR | A | 255 | 99.583 | 32.072 | 6.298 | 1.00 | 32.21 |
| ATOM | 2393 | CZ | TYR | A | 255 | 100.750 | 32.325 | 5.607 | 1.00 | 33.29 |
| ATOM | 2394 | OH | TYR | A | 255 | 100.741 | 33.230 | 4.573 | 1.00 | 38.59 |
| ATOM | 2396 | C | TYR | A | 255 | 100.559 | 28.955 | 11.195 | 1.00 | 31.73 |
| ATOM | 2397 | O | TYR | A | 255 | 101.156 | 27.892 | 11.330 | 1.00 | 32.57 |
| ATOM | 2398 | N | LEU | A | 256 | 99.490 | 29.279 | 11.914 | 1.00 | 31.36 |
| ATOM | 2400 | CA | LEU | A | 256 | 98.916 | 28.370 | 12.893 | 1.00 | 29.13 |
| ATOM | 2401 | CB | LEU | A | 256 | 98.878 | 28.992 | 14.280 | 1.00 | 28.40 |
| ATOM | 2402 | CG | LEU | A | 256 | 100.206 | 29.125 | 15.014 | 1.00 | 28.89 |
| ATOM | 2403 | CD1 | LEU | A | 256 | 101.045 | 30.208 | 14.375 | 1.00 | 32.20 |
| ATOM | 2404 | CD2 | LEU | A | 256 | 99.938 | 29.462 | 16.471 | 1.00 | 32.12 |
| ATOM | 2405 | C | LEU | A | 256 | 97.509 | 27.984 | 12.489 | 1.00 | 30.86 |
| ATOM | 2406 | O | LEU | A | 256 | 96.748 | 28.814 | 11.987 | 1.00 | 30.77 |
| ATOM | 2407 | N | GLY | A | 257 | 97.203 | 26.701 | 12.652 | 1.00 | 31.62 |
| ATOM | 2409 | CA | GLY | A | 257 | 95.888 | 26.180 | 12.345 | 1.00 | 29.36 |
| ATOM | 2410 | C | GLY | A | 257 | 95.287 | 25.827 | 13.688 | 1.00 | 29.99 |
| ATOM | 2411 | O | GLY | A | 257 | 95.662 | 24.830 | 14.295 | 1.00 | 32.08 |
| ATOM | 2412 | N | LEU | A | 258 | 94.402 | 26.686 | 14.177 | 1.00 | 29.99 |
| ATOM | 2414 | CA | LEU | A | 258 | 93.742 | 26.507 | 15.463 | 1.00 | 26.58 |
| ATOM | 2415 | CB | LEU | A | 258 | 93.562 | 27.871 | 16.101 | 1.00 | 26.90 |
| ATOM | 2416 | CG | LEU | A | 258 | 94.866 | 28.668 | 16.049 | 1.00 | 28.57 |
| ATOM | 2417 | CD1 | LEU | A | 258 | 94.583 | 30.144 | 16.176 | 1.00 | 29.14 |
| ATOM | 2418 | CD2 | LEU | A | 258 | 95.808 | 28.177 | 17.139 | 1.00 | 29.78 |
| ATOM | 2419 | C | LEU | A | 258 | 92.397 | 25.838 | 15.253 | 1.00 | 28.27 |
| ATOM | 2420 | O | LEU | A | 258 | 91.404 | 26.499 | 14.936 | 1.00 | 29.90 |
| ATOM | 2421 | N | SER | A | 259 | 92.370 | 24.523 | 15.434 | 1.00 | 28.81 |
| ATOM | 2423 | CA | SER | A | 259 | 91.164 | 23.726 | 15.229 | 1.00 | 30.63 |
| ATOM | 2424 | CB | SER | A | 259 | 91.491 | 22.237 | 15.348 | 1.00 | 31.03 |
| ATOM | 2425 | OG | SER | A | 259 | 91.783 | 21.889 | 16.689 | 1.00 | 33.43 |
| ATOM | 2427 | C | SER | A | 259 | 90.003 | 24.041 | 16.152 | 1.00 | 31.95 |
| ATOM | 2428 | O | SER | A | 259 | 90.184 | 24.600 | 17.231 | 1.00 | 33.18 |
| ATOM | 2429 | N | ASP | A | 260 | 88.808 | 23.637 | 15.729 | 1.00 | 32.89 |
| ATOM | 2431 | CA | ASP | A | 260 | 87.616 | 23.844 | 16.530 | 1.00 | 31.93 |
| ATOM | 2432 | CB | ASP | A | 260 | 86.326 | 23.521 | 15.739 | 1.00 | 33.23 |
| ATOM | 2433 | CG | ASP | A | 260 | 86.327 | 22.127 | 15.080 | 1.00 | 32.26 |
| ATOM | 2434 | OD1 | ASP | A | 260 | 87.138 | 21.246 | 15.425 | 1.00 | 33.22 |
| ATOM | 2435 | OD2 | ASP | A | 260 | 85.473 | 21.904 | 14.203 | 1.00 | 32.09 |
| ATOM | 2436 | C | ASP | A | 260 | 87.725 | 23.015 | 17.807 | 1.00 | 30.67 |
| ATOM | 2437 | O | ASP | A | 260 | 87.234 | 23.417 | 18.856 | 1.00 | 34.53 |
| ATOM | 2438 | N | TYR | A | 261 | 88.414 | 21.881 | 17.715 | 1.00 | 27.04 |
| ATOM | 2440 | CA | TYR | A | 261 | 88.629 | 20.983 | 18.847 | 1.00 | 26.39 |
| ATOM | 2441 | CB | TYR | A | 261 | 89.490 | 19.789 | 18.382 | 1.00 | 27.42 |
| ATOM | 2442 | CG | TYR | A | 261 | 90.064 | 18.885 | 19.463 | 1.00 | 26.82 |
| ATOM | 2443 | CD1 | TYR | A | 261 | 91.304 | 19.157 | 20.038 | 1.00 | 29.26 |
| ATOM | 2444 | CE1 | TYR | A | 261 | 91.855 | 18.318 | 21.006 | 1.00 | 30.93 |
| ATOM | 2445 | CD2 | TYR | A | 261 | 89.386 | 17.745 | 19.886 | 1.00 | 26.82 |
| ATOM | 2446 | CE2 | TYR | A | 261 | 89.927 | 16.898 | 20.853 | 1.00 | 28.99 |
| ATOM | 2447 | CZ | TYR | A | 261 | 91.163 | 17.194 | 21.408 | 1.00 | 32.15 |
| ATOM | 2448 | OH | TYR | A | 261 | 91.715 | 16.368 | 22.364 | 1.00 | 36.84 |
| ATOM | 2450 | C | TYR | A | 261 | 89.345 | 21.796 | 19.917 | 1.00 | 25.35 |
| ATOM | 2451 | O | TYR | A | 261 | 88.939 | 21.824 | 21.076 | 1.00 | 31.74 |
| ATOM | 2452 | N | PHE | A | 262 | 90.361 | 22.525 | 19.488 | 1.00 | 23.54 |
| ATOM | 2454 | CA | PHE | A | 262 | 91.151 | 23.361 | 20.368 | 1.00 | 21.85 |
| ATOM | 2455 | CB | PHE | A | 262 | 92.141 | 24.158 | 19.514 | 1.00 | 19.79 |
| ATOM | 2456 | CG | PHE | A | 262 | 92.923 | 25.184 | 20.275 | 1.00 | 20.92 |
| ATOM | 2457 | CD1 | PHE | A | 262 | 93.830 | 24.806 | 21.253 | 1.00 | 22.00 |
| ATOM | 2458 | CD2 | PHE | A | 262 | 92.761 | 26.536 | 20.002 | 1.00 | 20.09 |
| ATOM | 2459 | CE1 | PHE | A | 262 | 94.558 | 25.759 | 21.943 | 1.00 | 19.37 |
| ATOM | 2460 | CE2 | PHE | A | 262 | 93.488 | 27.497 | 20.693 | 1.00 | 18.46 |
| ATOM | 2461 | CZ | PHE | A | 262 | 94.386 | 27.107 | 21.660 | 1.00 | 18.18 |
| ATOM | 2462 | C | PHE | A | 262 | 90.244 | 24.290 | 21.165 | 1.00 | 21.70 |
| ATOM | 2463 | O | PHE | A | 262 | 90.236 | 24.266 | 22.391 | 1.00 | 22.47 |
| ATOM | 2464 | N | PHE | A | 263 | 89.432 | 25.060 | 20.457 | 1.00 | 24.02 |
| ATOM | 2466 | CA | PHE | A | 263 | 88.522 | 26.014 | 21.079 | 1.00 | 23.57 |
| ATOM | 2467 | CB | PHE | A | 263 | 87.843 | 26.845 | 20.005 | 1.00 | 22.46 |
| ATOM | 2468 | CG | PHE | A | 263 | 88.783 | 27.712 | 19.241 | 1.00 | 21.39 |
| ATOM | 2469 | CD1 | PHE | A | 263 | 89.462 | 28.740 | 19.870 | 1.00 | 25.06 |
| ATOM | 2470 | CD2 | PHE | A | 263 | 88.982 | 27.510 | 17.887 | 1.00 | 23.84 |
| ATOM | 2471 | CE1 | PHE | A | 263 | 90.326 | 29.555 | 19.159 | 1.00 | 26.31 |
| ATOM | 2472 | CE2 | PHE | A | 263 | 89.843 | 28.319 | 17.169 | 1.00 | 25.34 |
| ATOM | 2473 | CZ | PHE | A | 263 | 90.514 | 29.345 | 17.804 | 1.00 | 25.18 |
| ATOM | 2474 | C | PHE | A | 263 | 87.457 | 25.378 | 21.933 | 1.00 | 25.63 |
| ATOM | 2475 | O | PHE | A | 263 | 87.103 | 25.892 | 22.992 | 1.00 | 29.04 |
| ATOM | 2476 | N | ASN | A | 264 | 86.926 | 24.264 | 21.463 | 1.00 | 27.70 |
| ATOM | 2478 | CA | ASN | A | 264 | 85.882 | 23.577 | 22.193 | 1.00 | 26.71 |
| ATOM | 2479 | CB | ASN | A | 264. | 85.136 | 22.617 | 21.278 | 1.00 | 26.91 |
| ATOM | 2480 | CG | ASN | A | 264 | 84.183 | 23.338 | 20.345 | 1.00 | 26.18 |
| ATOM | 2481 | OD1 | ASN | A | 264 | 83.115 | 23.779 | 20.755 | 1.00 | 29.25 |
| ATOM | 2482 | ND2 | ASN | A | 264 | 84.563 | 23.462 | 19.087 | 1.00 | 30.59 |
| ATOM | 2485 | C | ASN | A | 264 | 86.359 | 22.898 | 23.470 | 1.00 | 26.40 |
| ATOM | 2486 | O | ASN | A | 264 | 85.587 | 22.763 | 24.414 | 1.00 | 27.80 |
| ATOM | 2487 | N | THR | A | 265 | 87.628 | 22.498 | 23.527 | 1.00 | 26.30 |
| ATOM | 2489 | CA | THR | A | 265 | 88.154 | 21.880 | 24.748 | 1.00 | 24.34 |
| ATOM | 2490 | CB | THR | A | 265 | 89.572 | 21.298 | 24.559 | 1.00 | 24.55 |
| ATOM | 2491 | OG1 | THR | A | 265 | 90.453 | 22.296 | 24.033 | 1.00 | 26.59 |
| ATOM | 2493 | CG2 | THR | A | 265 | 89.532 | 20.129 | 23.619 | 1.00 | 21.96 |
| ATOM | 2494 | C | THR | A | 265 | 88.175 | 22.924 | 25.865 | 1.00 | 23.53 |
| ATOM | 2495 | O | THR | A | 265 | 88.045 | 22.602 | 27.041 | 1.00 | 26.65 |
| ATOM | 2496 | N | ALA | A | 266 | 88.309 | 24.186 | 25.483 | 1.00 | 24.00 |
| ATOM | 2498 | CA | ALA | A | 266 | 88.315 | 25.276 | 26.437 | 1.00 | 22.35 |
| ATOM | 2499 | CB | ALA | A | 266 | 88.582 | 26.590 | 25.723 | 1.00 | 21.75 |
| ATOM | 2500 | C | ALA | A | 266 | 86.956 | 25.310 | 27.113 | 1.00 | 23.18 |
| ATOM | 2501 | O | ALA | A | 266 | 86.865 | 25.297 | 28.336 | 1.00 | 27.51 |
| ATOM | 2502 | N | GLY | A | 267 | 85.897 | 25.286 | 26.311 | 1.00 | 24.69 |
| ATOM | 2504 | CA | GLY | A | 267 | 84.549 | 25.323 | 26.852 | 1.00 | 24.20 |
| ATOM | 2505 | C | GLY | A | 267 | 84.251 | 24.188 | 27.812 | 1.00 | 27.46 |
| ATOM | 2506 | O | GLY | A | 267 | 83.637 | 24.381 | 28.869 | 1.00 | 27.80 |
| ATOM | 2507 | N | LEU | A | 268 | 84.699 | 22.997 | 27.445 | 1.00 | 28.36 |
| ATOM | 2509 | CA | LEU | A | 268 | 84.493 | 21.816 | 28.262 | 1.00 | 29.18 |
| ATOM | 2510 | CB | LEU | A | 268 | 84.996 | 20.580 | 27.520 | 1.00 | 32.75 |
| ATOM | 2511 | CG | LEU | A | 268 | 84.728 | 19.224 | 28.169 | 1.00 | 37.10 |
| ATOM | 2512 | CD1 | LEU | A | 268 | 83.274 | 18.841 | 27.978 | 1.00 | 38.10 |
| ATOM | 2513 | CD2 | LEU | A | 268 | 85.633 | 18.180 | 27.544 | 1.00 | 41.14 |
| ATOM | 2514 | C | LEU | A | 268 | 85.209 | 21.940 | 29.596 | 1.00 | 27.77 |
| ATOM | 2515 | O | LEU | A | 268 | 84.623 | 21.696 | 30.644 | 1.00 | 31.78 |
| ATOM | 2516 | N | VAL | A | 269 | 86.469 | 22.344 | 29.558 | 1.00 | 26.94 |
| ATOM | 2518 | CA | VAL | A | 269 | 87.256 | 22.478 | 30.774 | 1.00 | 26.89 |
| ATOM | 2519 | CB | VAL | A | 269 | 88.752 | 22.720 | 30.448 | 1.00 | 24.63 |
| ATOM | 2520 | CG1 | VAL | A | 269 | 89.491 | 23.244 | 31.656 | 1.00 | 22.76 |
| ATOM | 2521 | CG2 | VAL | A | 269 | 89.382 | 21.416 | 30.007 | 1.00 | 21.59 |
| ATOM | 2522 | C | VAL | A | 269 | 86.722 | 23.535 | 31.740 | 1.00 | 28.84 |
| ATOM | 2523 | O | VAL | A | 269 | 86.687 | 23.309 | 32.948 | 1.00 | 29.66 |
| ATOM | 2524 | N | TYR | A | 270 | 86.298 | 24.681 | 31.219 | 1.00 | 30.57 |
| ATOM | 2526 | CA | TYR | A | 270 | 85.769 | 25.734 | 32.080 | 1.00 | 28.60 |
| ATOM | 2527 | CB | TYR | A | 270 | 85.753 | 27.079 | 31.350 | 1.00 | 28.80 |
| ATOM | 2528 | CG | TYR | A | 270 | 87.111 | 27.734 | 31.265 | 1.00 | 27.47 |
| ATOM | 2529 | CD1 | TYR | A | 270. | 87.766 | 28.169 | 32.415 | 1.00 | 25.23 |
| ATOM | 2530 | CE1 | TYR | A | 270 | 89.021 | 28.754 | 32.343 | 1.00 | 26.36 |
| ATOM | 2531 | CD2 | TYR | A | 270 | 87.750 | 27.906 | 30.038 | 1.00 | 29.53 |
| ATOM | 2532 | CE2 | TYR | A | 270 | 89.008 | 28.494 | 29.959 | 1.00 | 25.30 |
| ATOM | 2533 | CZ | TYR | A | 270 | 89.632 | 28.912 | 31.114 | 1.00 | 24.84 |
| ATOM | 2534 | OH | TYR | A | 270 | 90.877 | 29.492 | 31.047 | 1.00 | 30.89 |
| ATOM | 2536 | C | TYR | A | 270 | 84.385 | 25.405 | 32.631 | 1.00 | 29.08 |
| ATOM | 2537 | O | TYR | A | 270 | 84.034 | 25.836 | 33.729 | 1.00 | 28.17 |
| ATOM | 2538 | N | GLN | A | 271 | 83.609 | 24.628 | 31.882 | 1.00 | 30.32 |
| ATOM | 2540 | CA | GLN | A | 271 | 82.269 | 24.249 | 32.316 | 1.00 | 29.24 |
| ATOM | 2541 | CB | GLN | A | 271 | 81.464 | 23.689 | 31.139 | 1.00 | 27.20 |
| ATOM | 2542 | CG | GLN | A | 271 | 79.957 | 23.680 | 31.357 | 1.00 | 27.60 |
| ATOM | 2543 | CD | GLN | A | 271 | 79.427 | 22.387 | 31.946 | 1.00 | 31.03 |
| ATOM | 2544 | OE1 | GLN | A | 271 | 79.982 | 21.306 | 31.724 | 1.00 | 35.11 |
| ATOM | 2545 | NE2 | GLN | A | 271 | 78.318 | 22.484 | 32.668 | 1.00 | 28.89 |
| ATOM | 2548 | C | GLN | A | 271 | 82.329 | 23.226 | 33.438 | 1.00 | 30.41 |
| ATOM | 2549 | O | GLN | A | 271 | 81.839 | 23.473 | 34.539 | 1.00 | 32.81 |
| ATOM | 2550 | N | GLU | A | 272 | 82.976 | 22.098 | 33.168 | 1.00 | 33.61 |
| ATOM | 2552 | CA | GLU | A | 272 | 83.096 | 21.011 | 34.133 | 1.00 | 36.97 |
| ATOM | 2553 | CB | GLU | A | 272 | 83.807 | 19.822 | 33.492 | 1.00 | 40.58 |
| ATOM | 2554 | CG | GLU | A | 272 | 83.084 | 19.264 | 32.274 | 1.00 | 48.51 |
| ATOM | 2555 | CD | GLU | A | 272 | 83.781 | 18.051 | 31.674 | 1.00 | 55.01 |
| ATOM | 2556 | OE1 | GLU | A | 272 | 84.998 | 18.129 | 31.405 | 1.00 | 59.64 |
| ATOM | 2557 | OE2 | GLU | A | 272 | 83.112 | 17.016 | 31.468 | 1.00 | 57.25 |
| ATOM | 2558 | C | GLU | A | 272 | 83.761 | 21.376 | 35.460 | 1.00 | 36.60 |
| ATOM | 2559 | O | GLU | A | 272 | 83.674 | 20.622 | 36.425 | 1.00 | 40.35 |
| ATOM | 2560 | N | ALA | A | 273 | 84.429 | 22.522 | 35.512 | 1.00 | 35.77 |
| ATOM | 2562 | CA | ALA | A | 273 | 85.079 | 22.970 | 36.741 | 1.00 | 34.34 |
| ATOM | 2563 | CB | ALA | A | 273 | 86.316 | 23.798 | 36.418 | 1.00 | 32.87 |
| ATOM | 2564 | C | ALA | A | 273 | 84.104 | 23.778 | 37.594 | 1.00 | 34.54 |
| ATOM | 2565 | O | ALA | A | 273 | 84.459 | 24.252 | 38.679 | 1.00 | 35.72 |
| ATOM | 2566 | N | GLY | A | 274 | 82.879 | 23.930 | 37.086 | 1.00 | 35.04 |
| ATOM | 2568 | CA | GLY | A | 274 | 81.831 | 24.663 | 37.779 | 1.00 | 35.01 |
| ATOM | 2569 | C | GLY | A | 274 | 82.092 | 26.147 | 37.930 | 1.00 | 34.45 |
| ATOM | 2570 | O | GLY | A | 274 | 81.640 | 26.761 | 38.897 | 1.00 | 36.89 |
| ATOM | 2571 | N | VAL | A | 275 | 82.752 | 26.742 | 36.944 | 1.00 | 34.05 |
| ATOM | 2573 | CA | VAL | A | 275 | 83.083 | 28.157 | 37.018 | 1.00 | 34.43 |
| ATOM | 2574 | CB | VAL | A | 275 | 84.600 | 28.381 | 36.778 | 1.00 | 35.67 |
| ATOM | 2575 | CG1 | VAL | A | 275 | 84.898 | 28.616 | 35.308 | 1.00 | 37.90 |
| ATOM | 2576 | CG2 | VAL | A | 275 | 85.105 | 29.519 | 37.638 | 1.00 | 38.34 |
| ATOM | 2577 | C | VAL | A | 275 | 82.236 | 29.078 | 36.135 | 1.00 | 33.65 |
| ATOM | 2578 | O | VAL | A | 275 | 82.211 | 30.286 | 36.343 | 1.00 | 35.12 |
| ATOM | 2579 | N | LEU | A | 276 | 81.527 | 28.523 | 35.159 | 1.00 | 32.82 |
| ATOM | 2581 | CA | LEU | A | 276 | 80.683 | 29.352 | 34.307 | 1.00 | 29.38 |
| ATOM | 2582 | CB | LEU | A | 276 | 80.478 | 28.695 | 32.936 | 1.00 | 27.64 |
| ATOM | 2583 | CG | LEU | A | 276 | 81.739 | 28.482 | 32.082 | 1.00 | 25.37 |
| ATOM | 2584 | CD1 | LEU | A | 276 | 81.370 | 27.883 | 30.757 | 1.00 | 24.34 |
| ATOM | 2585 | CD2 | LEU | A | 276 | 82.459 | 29.793 | 31.858 | 1.00 | 28.39 |
| ATOM | 2586 | C | LEU | A | 276 | 79.363 | 29.548 | 35.049 | 1.00 | 29.83 |
| ATOM | 2587 | O | LEU | A | 276 | 78.323 | 29.007 | 34.674 | 1.00 | 30.32 |
| ATOM | 2588 | N | LYS | A | 277 | 79.443 | 30.271 | 36.158 | 1.00 | 29.10 |
| ATOM | 2590 | CA | LYS | A | 277 | 78.294 | 30.546 | 37.004 | 1.00 | 27.79 |
| ATOM | 2591 | CB | LYS | A | 277 | 78.327 | 29.656 | 38.246 | 1.00 | 29.34 |
| ATOM | 2592 | CG | LYS | A | 277 | 78.211 | 28.183 | 37.956 | 1.00 | 33.13 |
| ATOM | 2593 | CD | LYS | A | 277 | 78.269 | 27.369 | 39.230 | 1.00 | 39.28 |
| ATOM | 2594 | CE | LYS | A | 277 | 78.064 | 25.888 | 38.930 | 1.00 | 44.23 |
| ATOM | 2595 | NZ | LYS | A | 277 | 78.321 | 25.022 | 40.119 | 1.00 | 46.99 |
| ATOM | 2599 | C | LYS | A | 277 | 78.386 | 32.002 | 37.420 | 1.00 | 26.55 |
| ATOM | 2600 | O | LYS | A | 277 | 79.474 | 32.556 | 37.516 | 1.00 | 28.05 |
| ATOM | 2601 | N | MET | A | 278 | 77.251 | 32.608 | 37.723 | 1.00 | 25.90 |
| ATOM | 2603 | CA | MET | A | 278 | 77.251 | 33.999 | 38.111 | 1.00 | 26.31 |
| ATOM | 2604 | CB | MET | A | 278 | 77.350 | 34.854 | 36.864 | 1.00 | 25.28 |
| ATOM | 2605 | CG | MET | A | 278 | 77.496 | 36.310 | 37.129 | 1.00 | 30.09 |
| ATOM | 2606 | SD | MET | A | 278 | 77.760 | 37.127 | 35.586 | 1.00 | 37.76 |
| ATOM | 2607 | CE | MET | A | 278 | 77.452 | 38.822 | 36.056 | 1.00 | 34.82 |
| ATOM | 2608 | C | MET | A | 278 | 75.985 | 34.338 | 38.872 | 1.00 | 28.79 |
| ATOM | 2609 | O | MET | A | 278 | 74.910 | 33.858 | 38.533 | 1.00 | 30.81 |
| ATOM | 2610 | N | THR | A | 279 | 76.123 | 35.134 | 39.925 | 1.00 | 30.03 |
| ATOM | 2612 | CA | THR | A | 279 | 74.987 | 35.544 | 40.728 | 1.00 | 29.69 |
| ATOM | 2613 | CB | THR | A | 279 | 75.242 | 35.277 | 42.211 | 1.00 | 29.00 |
| ATOM | 2614 | OG1 | THR | A | 279 | 75.286 | 33.864 | 42.431 | 1.00 | 31.54 |
| ATOM | 2616 | CG2 | THR | A | 279 | 74.140 | 35.863 | 43.060 | 1.00 | 28.67 |
| ATOM | 2617 | C | THR | A | 279 | 74.714 | 37.025 | 40.515 | 1.00 | 31.93 |
| ATOM | 2618 | O | THR | A | 279 | 75.615 | 37.856 | 40.644 | 1.00 | 32.91 |
| ATOM | 2619 | N | LEU | A | 280 | 73.482 | 37.338 | 40.128 | 1.00 | 33.14 |
| ATOM | 2621 | CA | LEU | A | 280 | 73.062 | 38.715 | 39.893 | 1.00 | 33.77 |
| ATOM | 2622 | CB | LEU | A | 280 | 72.268 | 38.826 | 38.581 | 1.00 | 34.16 |
| ATOM | 2623 | CG | LEU | A | 280 | 72.930 | 38.757 | 37.194 | 1.00 | 33.72 |
| ATOM | 2624 | CD1 | LEU | A | 280 | 74.391 | 38.394 | 37.271 | 1.00 | 33.08 |
| ATOM | 2625 | CD2 | LEU | A | 280 | 72.186 | 37.766 | 36.333 | 1.00 | 30.96 |
| ATOM | 2626 | C | LEU | A | 280 | 72.196 | 39.173 | 41.064 | 1.00 | 35.73 |
| ATOM | 2627 | O | LEU | A | 280 | 71.223 | 38.508 | 41.438 | 1.00 | 33.09 |
| ATOM | 2628 | N | ARG | A | 281 | 72.584 | 40.290 | 41.666 | 1.00 | 39.95 |
| ATOM | 2630 | CA | ARG | A | 281 | 71.864 | 40.860 | 42.796 | 1.00 | 42.55 |
| ATOM | 2631 | CB | ARG | A | 281 | 72.823 | 41.091 | 43.975 | 1.00 | 44.48 |
| ATOM | 2632 | CG | ARG | A | 281 | 73.472 | 39.824 | 44.534 | 1.00 | 47.55 |
| ATOM | 2633 | CD | ARG | A | 281 | 74.406 | 40.109 | 45.712 | 1.00 | 50.80 |
| ATOM | 2634 | NE | ARG | A | 281 | 75.660 | 40.757 | 45.321 | 1.00 | 57.47 |
| ATOM | 2636 | CZ | ARG | A | 281 | 76.844 | 40.145 | 45.263 | 1.00 | 61.45 |
| ATOM | 2637 | NH1 | ARG | A | 281 | 76.956 | 38.852 | 45.552 | 1.00 | 62.83 |
| ATOM | 2640 | NH2 | ARG | A | 281 | 77.925 | 40.825 | 44.901 | 1.00 | 62.95 |
| ATOM | 2643 | C | ARG | A | 281 | 71.255 | 42.190 | 42.354 | 1.00 | 44.72 |
| ATOM | 2644 | O | ARG | A | 281 | 71.782 | 42.854 | 41.458 | 1.00 | 46.61 |
| ATOM | 2645 | N | ASP | A | 282 | 70.179 | 42.601 | 43.014 | 1.00 | 46.42 |
| ATOM | 2647 | CA | ASP | A | 282 | 69.491 | 43.849 | 42.702 | 1.00 | 47.87 |
| ATOM | 2648 | CB | ASP | A | 282 | 68.284 | 44.039 | 43.634 | 1.00 | 49.38 |
| ATOM | 2649 | CG | ASP | A | 282 | 67.472 | 45.280 | 43.302 | 1.00 | 51.57 |
| ATOM | 2650 | OD1 | ASP | A | 282 | 66.898 | 45.349 | 42.195 | 1.00 | 54.37 |
| ATOM | 2651 | OD2 | ASP | A | 282 | 67.408 | 46.191 | 44.150 | 1.00 | 54.52 |
| ATOM | 2652 | C | ASP | A | 282 | 70.394 | 45.080 | 42.762 | 1.00 | 48.67 |
| ATOM | 2653 | O | ASP | A | 282 | 70.090 | 46.089 | 42.141 | 1.00 | 51.13 |
| ATOM | 2654 | N | ASP | A | 283 | 71.506 | 44.996 | 43.488 | 1.00 | 49.71 |
| ATOM | 2656 | CA | ASP | A | 283 | 72.427 | 46.128 | 43.608 | 1.00 | 50.78 |
| ATOM | 2657 | CB | ASP | A | 283 | 73.111 | 46.138 | 44.987 | 1.00 | 52.25 |
| ATOM | 2658 | CG | ASP | A | 283 | 73.994 | 44.920 | 45.224 | 1.00 | 55.58 |
| ATOM | 2659 | OD1 | ASP | A | 283 | 75.189 | 44.953 | 44.847 | 1.00 | 58.60 |
| ATOM | 2660 | OD2 | ASP | A | 283 | 73.498 | 43.935 | 45.810 | 1.00 | 58.13 |
| ATOM | 2661 | C | ASP | A | 283 | 73.474 | 46.236 | 42.492 | 1.00 | 51.35 |
| ATOM | 2662 | O | ASP | A | 283 | 74.362 | 47.086 | 42.543 | 1.00 | 52.74 |
| ATOM | 2663 | N | MET | A | 284 | 73.395 | 45.359 | 41.500 | 1.00 | 51.07 |
| ATOM | 2665 | CA | MET | A | 284 | 74.342 | 45.404 | 40.399 | 1.00 | 50.50 |
| ATOM | 2666 | CB | MET | A | 284 | 74.629 | 43.994 | 39.887 | 1.00 | 48.87 |
| ATOM | 2667 | CG | MET | A | 284 | 75.169 | 43.058 | 40.956 | 1.00 | 46.76 |
| ATOM | 2668 | SD | MET | A | 284 | 75.494 | 41.398 | 40.353 | 1.00 | 43.76 |
| ATOM | 2669 | CE | MET | A | 284 | 76.804 | 40.895 | 41.457 | 1.00 | 47.06 |
| ATOM | 2670 | C | MET | A | 284 | 73.775 | 46.283 | 39.289 | 1.00 | 53.22 |
| ATOM | 2671 | O | MET | A | 284 | 74.515 | 46.824 | 38.466 | 1.00 | 55.85 |
| ATOM | 2672 | N | ILE | A | 285 | 72.455 | 46.433 | 39.285 | 1.00 | 55.37 |
| ATOM | 2674 | CA | ILE | A | 285 | 71.761 | 47.252 | 38.295 | 1.00 | 57.52 |
| ATOM | 2675 | CB | ILE | A | 285 | 70.287 | 46.792 | 38.141 | 1.00 | 55.38 |
| ATOM | 2676 | CG2 | ILE | A | 285 | 69.583 | 47.611 | 37.070 | 1.00 | 56.70 |
| ATOM | 2677 | CG1 | ILE | A | 285 | 70.235 | 45.303 | 37.783 | 1.00 | 53.28 |
| ATOM | 2678 | CD1 | ILE | A | 285 | 68.838 | 44.723 | 37.732 | 1.00 | 50.41 |
| ATOM | 2679 | C | ILE | A | 285 | 71.787 | 48.707 | 38.776 | 1.00 | 59.50 |
| ATOM | 2680 | O | ILE | A | 285 | 71.358 | 48.998 | 39.889 | 1.00 | 60.66 |
| ATOM | 2681 | N | PRO | A | 286 | 72.304 | 49.635 | 37.951 | 1.00 | 61.05 |
| ATOM | 2682 | CD | PRO | A | 286 | 72.787 | 49.459 | 36.573 | 1.00 | 61.08 |
| ATOM | 2683 | CA | PRO | A | 286 | 72.360 | 51.048 | 38.350 | 1.00 | 62.59 |
| ATOM | 2684 | CB | PRO | A | 286 | 72.862 | 51.748 | 37.083 | 1.00 | 62.24 |
| ATOM | 2685 | CG | PRO | A | 286 | 72.481 | 50.801 | 35.974 | 1.00 | 63.04 |
| ATOM | 2686 | C | PRO | A | 286 | 71.004 | 51.583 | 38.817 | 1.00 | 64.15 |
| ATOM | 2687 | O | PRO | A | 286 | 69.986 | 51.400 | 38.154 | 1.00 | 63.39 |
| ATOM | 2688 | N | LYS | A | 287 | 71.016 | 52.268 | 39.956 | 1.00 | 67.25 |
| ATOM | 2690 | CA | LYS | A | 287 | 69.813 | 52.826 | 40.582 | 1.00 | 69.68 |
| ATOM | 2691 | CB | LYS | A | 287 | 70.204 | 53.622 | 41.838 | 1.00 | 72.51 |
| ATOM | 2692 | CG | LYS | A | 287 | 71.285 | 54.675 | 41.607 | 1.00 | 75.76 |
| ATOM | 2693 | CD | LYS | A | 287 | 71.483 | 55.570 | 42.826 | 1.00 | 78.18 |
| ATOM | 2694 | CE | LYS | A | 287 | 72.474 | 56.697 | 42.534 | 1.00 | 79.24 |
| ATOM | 2695 | NZ | LYS | A | 287 | 72.603 | 57.663 | 43.666 | 1.00 | 79.25 |
| ATOM | 2699 | C | LYS | A | 287 | 68.845 | 53.653 | 39.724 | 1.00 | 69.37 |
| ATOM | 2700 | O | LYS | A | 287 | 67.735 | 53.962 | 40.165 | 1.00 | 69.08 |
| ATOM | 2701 | N | GLU | A | 288 | 69.242 | 53.996 | 38.505 | 1.00 | 68.49 |
| ATOM | 2703 | CA | GLU | A | 288 | 68.386 | 54.792 | 37.635 | 1.00 | 69.17 |
| ATOM | 2704 | CB | GLU | A | 288 | 69.152 | 56.004 | 37.094 | 1.00 | 70.77 |
| ATOM | 2705 | CG | GLU | A | 288 | 69.897 | 56.826 | 38.142 | 1.00 | 73.92 |
| ATOM | 2706 | CD | GLU | A | 288 | 71.290 | 56.292 | 38.461 | 1.00 | 76.03 |
| ATOM | 2707 | OE1 | GLU | A | 288 | 71.656 | 55.195 | 37.983 | 1.00 | 78.84 |
| ATOM | 2708 | OE2 | GLU | A | 288 | 72.031 | 56.981 | 39.196 | 1.00 | 76.47 |
| ATOM | 2709 | C | GLU | A | 288 | 67.851 | 53.965 | 36.469 | 1.00 | 68.68 |
| ATOM | 2710 | O | GLU | A | 288 | 68.012 | 54.345 | 35.310 | 1.00 | 70.05 |
| ATOM | 2711 | N | SER | A | 289 | 67.194 | 52.852 | 36.772 | 1.00 | 68.09 |
| ATOM | 2713 | CA | SER | A | 289 | 66.655 | 51.985 | 35.727 | 1.00 | 67.82 |
| ATOM | 2714 | CB | SER | A | 289 | 67.516 | 50.728 | 35.593 | 1.00 | 69.68 |
| ATOM | 2715 | OG | SER | A | 289 | 68.868 | 51.064 | 35.327 | 1.00 | 73.68 |
| ATOM | 2717 | C | SER | A | 289 | 65.202 | 51.590 | 35.977 | 1.00 | 66.67 |
| ATOM | 2718 | O | SER | A | 289 | 64.695 | 51.730 | 37.090 | 1.00 | 67.98 |
| ATOM | 2719 | N | ALA | A | 290 | 64.537 | 51.110 | 34.928 | 1.00 | 63.49 |
| ATOM | 2721 | CA | ALA | A | 290 | 63.144 | 50.691 | 35.027 | 1.00 | 60.33 |
| ATOM | 2722 | CB | ALA | A | 290 | 62.525 | 50.598 | 33.644 | 1.00 | 60.15 |
| ATOM | 2723 | C | ALA | A | 290 | 63.026 | 49.353 | 35.744 | 1.00 | 58.83 |
| ATOM | 2724 | O | ALA | A | 290 | 62.236 | 49.208 | 36.673 | 1.00 | 59.19 |
| ATOM | 2725 | N | PHE | A | 291 | 63.839 | 48.390 | 35.321 | 1.00 | 57.41 |
| ATOM | 2727 | CA | PHE | A | 291 | 63.829 | 47.043 | 35.894 | 1.00 | 55.46 |
| ATOM | 2728 | CB | PHE | A | 291 | 64.441 | 46.044 | 34.902 | 1.00 | 54.51 |
| ATOM | 2729 | CG | PHE | A | 291 | 63.658 | 45.890 | 33.622 | 1.00 | 52.78 |
| ATOM | 2730 | CD1 | PHE | A | 291 | 62.493 | 45.130 | 33.588 | 1.00 | 52.76 |
| ATOM | 2731 | CD2 | PHE | A | 291 | 64.085 | 46.505 | 32.451 | 1.00 | 52.05 |
| ATOM | 2732 | CE1 | PHE | A | 291 | 61.760 | 44.983 | 32.414 | 1.00 | 51.50 |
| ATOM | 2733 | CE2 | PHE | A | 291 | 63.360 | 46.364 | 31.270 | 1.00 | 52.10 |
| ATOM | 2734 | CZ | PHE | A | 291 | 62.194 | 45.601 | 31.255 | 1.00 | 51.58 |
| ATOM | 2735 | C | PHE | A | 291 | 64.519 | 46.912 | 37.260 | 1.00 | 53.52 |
| ATOM | 2736 | O | PHE | A | 291 | 65.541 | 47.547 | 37.524 | 1.00 | 53.59 |
| ATOM | 2737 | N | ARG | A | 292 | 63.966 | 46.046 | 38.104 | 1.00 | 50.17 |
| ATOM | 2739 | CA | ARG | A | 292 | 64.494 | 45.799 | 39.438 | 1.00 | 46.66 |
| ATOM | 2740 | CB | ARG | A | 292 | 63.677 | 46.562 | 40.481 | 1.00 | 46.93 |
| ATOM | 2741 | CG | ARG | A | 292 | 63.793 | 48.065 | 40.434 | 1.00 | 47.97 |
| ATOM | 2742 | CD | ARG | A | 292 | 64.964 | 48.536 | 41.256 | 1.00 | 49.34 |
| ATOM | 2743 | NE | ARG | A | 292 | 65.959 | 49.215 | 40.438 | 1.00 | 51.19 |
| ATOM | 2745 | CZ | ARG | A | 292 | 67.266 | 48.993 | 40.521 | 1.00 | 52.32 |
| ATOM | 2746 | NH1 | ARG | A | 292 | 67.735 | 48.102 | 41.385 | 1.00 | 52.06 |
| ATOM | 2749 | NH2 | ARG | A | 292 | 68.106 | 49.674 | 39.750 | 1.00 | 52.32 |
| ATOM | 2752 | C | ARG | A | 292 | 64.317 | 44.322 | 39.715 | 1.00 | 46.15 |
| ATOM | 2753 | O | ARG | A | 292 | 63.333 | 43.720 | 39.278 | 1.00 | 47.65 |
| ATOM | 2754 | N | LEU | A | 293 | 65.278 | 43.725 | 40.409 | 1.00 | 44.69 |
| ATOM | 2756 | CA | LEU | A | 293 | 65.169 | 42.320 | 40.769 | 1.00 | 42.76 |
| ATOM | 2757 | CB | LEU | A | 293 | 66.546 | 41.648 | 40.819 | 1.00 | 37.95 |
| ATOM | 2758 | CG | LEU | A | 293 | 67.252 | 41.569 | 39.468 | 1.00 | 34.56 |
| ATOM | 2759 | CD1 | LEU | A | 293 | 68.649 | 41.034 | 39.630 | 1.00 | 33.82 |
| ATOM | 2760 | CD2 | LEU | A | 293 | 66.459 | 40.692 | 38.537 | 1.00 | 32.43 |
| ATOM | 2761 | C | LEU | A | 293 | 64.516 | 42.349 | 42.141 | 1.00 | 44.58 |
| ATOM | 2762 | O | LEU | A | 293 | 65.169 | 42.154 | 43.164 | 1.00 | 46.89 |
| ATOM | 2763 | N | THR | A | 294 | 63.239 | 42.716 | 42.150 | 1.00 | 44.93 |
| ATOM | 2765 | CA | THR | A | 294 | 62.453 | 42.804 | 43.370 | 1.00 | 45.20 |
| ATOM | 2766 | CB | THR | A | 294 | 62.220 | 44.271 | 43.781 | 1.00 | 44.99 |
| ATOM | 2767 | OG1 | THR | A | 294 | 61.502 | 44.955 | 42.748 | 1.00 | 45.39 |
| ATOM | 2769 | CG2 | THR | A | 294 | 63.547 | 44.983 | 44.012 | 1.00 | 46.79 |
| ATOM | 2770 | C | THR | A | 294 | 61.111 | 42.175 | 43.055 | 1.00 | 46.49 |
| ATOM | 2771 | O | THR | A | 294 | 60.639 | 42.269 | 41.922 | 1.00 | 48.98 |
| ATOM | 2772 | N | THR | A | 295 | 60.500 | 41.520 | 44.035 | 1.00 | 47.36 |
| ATOM | 2774 | CA | THR | A | 295 | 59.204 | 40.892 | 43.812 | 1.00 | 48.40 |
| ATOM | 2775 | CB | THR | A | 295 | 58.720 | 40.139 | 45.063 | 1.00 | 47.45 |
| ATOM | 2776 | OG1 | THR | A | 295 | 58.837 | 40.986 | 46.210 | 1.00 | 47.38 |
| ATOM | 2778 | CG2 | THR | A | 295 | 59.550 | 38.891 | 45.287 | 1.00 | 47.66 |
| ATOM | 2779 | C | THR | A | 295 | 58.179 | 41.952 | 43.410 | 1.00 | 49.59 |
| ATOM | 2780 | O | THR | A | 295 | 57.292 | 41.693 | 42.599 | 1.00 | 50.18 |
| ATOM | 2781 | N | SER | A | 296 | 58.347 | 43.157 | 43.950 | 1.00 | 51.29 |
| ATOM | 2783 | CA | SER | A | 296 | 57.468 | 44.288 | 43.667 | 1.00 | 52.78 |
| ATOM | 2784 | CB | SER | A | 296 | 57.978 | 45.538 | 44.390 | 1.00 | 54.54 |
| ATOM | 2785 | OG | SER | A | 296 | 57.219 | 46.688 | 44.056 | 1.00 | 55.48 |
| ATOM | 2787 | C | SER | A | 296 | 57.412 | 44.551 | 42.172 | 1.00 | 52.76 |
| ATOM | 2788 | O | SER | A | 296 | 56.340 | 44.532 | 41.562 | 1.00 | 52.27 |
| ATOM | 2789 | N | PHE | A | 297 | 58.579 | 44.779 | 41.583 | 1.00 | 52.83 |
| ATOM | 2791 | CA | PHE | A | 297 | 58.656 | 45.033 | 40.160 | 1.00 | 53.94 |
| ATOM | 2792 | CB | PHE | A | 297 | 60.093 | 45.286 | 39.730 | 1.00 | 55.28 |
| ATOM | 2793 | CG | PHE | A | 297 | 60.217 | 45.651 | 38.293 | 1.00 | 57.93 |
| ATOM | 2794 | CD1 | PHE | A | 297 | 60.028 | 46.964 | 37.884 | 1.00 | 57.66 |
| ATOM | 2795 | CD2 | PHE | A | 297 | 60.459 | 44.673 | 37.334 | 1.00 | 59.49 |
| ATOM | 2796 | CE1 | PHE | A | 297 | 60.072 | 47.299 | 36.539 | 1.00 | 59.28 |
| ATOM | 2797 | CE2 | PHE | A | 297 | 60.503 | 44.997 | 35.990 | 1.00 | 59.71 |
| ATOM | 2798 | CZ | PHE | A | 297 | 60.309 | 46.315 | 35.590 | 1.00 | 59.93 |
| ATOM | 2799 | C | PHE | A | 297 | 58.090 | 43.837 | 39.408 | 1.00 | 54.31 |
| ATOM | 2800 | O | PHE | A | 297 | 57.245 | 43.990 | 38.530 | 1.00 | 54.87 |
| ATOM | 2801 | N | PHE | A | 298 | 58.542 | 42.643 | 39.776 | 1.00 | 55.31 |
| ATOM | 2803 | CA | PHE | A | 298 | 58.058 | 41.420 | 39.149 | 1.00 | 55.92 |
| ATOM | 2804 | CB | PHE | A | 298 | 58.793 | 40.199 | 39.709 | 1.00 | 52.95 |
| ATOM | 2805 | CG | PHE | A | 298 | 60.118 | 39.943 | 39.057 | 1.00 | 49.79 |
| ATOM | 2806 | CD1 | PHE | A | 298 | 61.094 | 40.934 | 39.019 | 1.00 | 46.14 |
| ATOM | 2807 | CD2 | PHE | A | 298 | 60.384 | 38.716 | 38.463 | 1.00 | 47.97 |
| ATOM | 2808 | CE1 | PHE | A | 298 | 62.312 | 40.709 | 38.400 | 1.00 | 44.36 |
| ATOM | 2809 | CE2 | PHE | A | 298 | 61.600 | 38.482 | 37.842 | 1.00 | 46.94 |
| ATOM | 2810 | CZ | PHE | A | 298 | 62.567 | 39.482 | 37.810 | 1.00 | 45.78 |
| ATOM | 2811 | C | PHE | A | 298 | 56.556 | 41.274 | 39.364 | 1.00 | 58.12 |
| ATOM | 2812 | O | PHE | A | 298 | 55.904 | 40.453 | 38.716 | 1.00 | 58.39 |
| ATOM | 2813 | N | GLY | A | 299 | 56.019 | 42.067 | 40.287 | 1.00 | 60.34 |
| ATOM | 2815 | CA | GLY | A | 299 | 54.599 | 42.042 | 40.576 | 1.00 | 62.61 |
| ATOM | 2816 | C | GLY | A | 299 | 53.777 | 42.541 | 39.404 | 1.00 | 64.03 |
| ATOM | 2817 | O | GLY | A | 299 | 52.622 | 42.156 | 39.236 | 1.00 | 64.07 |
| ATOM | 2818 | N | THR | A | 300 | 54.377 | 43.379 | 38.571 | 1.00 | 65.39 |
| ATOM | 2820 | CA | THR | A | 300 | 53.672 | 43.904 | 37.416 | 1.00 | 67.18 |
| ATOM | 2821 | CB | THR | A | 300 | 54.532 | 44.935 | 36.658 | 1.00 | 66.57 |
| ATOM | 2822 | OG1 | THR | A | 300 | 55.806 | 44.361 | 36.349 | 1.00 | 67.61 |
| ATOM | 2824 | CG2 | THR | A | 300 | 54.736 | 46.181 | 37.503 | 1.00 | 67.44 |
| ATOM | 2825 | C | THR | A | 300 | 53.258 | 42.785 | 36.460 | 1.00 | 69.21 |
| ATOM | 2826 | O | THR | A | 300 | 52.234 | 42.896 | 35.777 | 1.00 | 72.16 |
| ATOM | 2827 | N | PHE | A | 301 | 54.055 | 41.774 | 36.384 | 1.00 | 69.34 |
| ATOM | 2829 | CA | PHE | A | 301 | 53.704 | 40.675 | 35.483 | 1.00 | 69.26 |
| ATOM | 2830 | CB | PHE | A | 301 | 54.608 | 40.680 | 34.241 | 1.00 | 66.47 |
| ATOM | 2831 | CG | PHE | A | 301 | 56.070 | 40.882 | 34.532 | 1.00 | 64.06 |
| ATOM | 2832 | CD1 | PHE | A | 301 | 56.629 | 42.154 | 34.478 | 1.00 | 62.60 |
| ATOM | 2833 | CD2 | PHE | A | 301 | 56.895 | 39.806 | 34.838 | 1.00 | 63.14 |
| ATOM | 2834 | CE1 | PHE | A | 301 | 57.983 | 42.354 | 34.716 | 1.00 | 61.30 |
| ATOM | 2835 | CE2 | PHE | A | 301 | 58.251 | 39.995 | 35.077 | 1.00 | 61.28 |
| ATOM | 2836 | CZ | PHE | A | 301 | 58.795 | 41.274 | 35.016 | 1.00 | 61.18 |
| ATOM | 2837 | C | PHE | A | 301 | 53.635 | 39.268 | 36.062 | 1.00 | 71.49 |
| ATOM | 2838 | O | PHE | A | 301 | 53.032 | 38.380 | 35.458 | 1.00 | 71.98 |
| ATOM | 2839 | N | LEU | A | 302 | 54.199 | 39.072 | 37.250 | 1.00 | 74.67 |
| ATOM | 2841 | CA | LEU | A | 302 | 54.198 | 37.748 | 37.866 | 1.00 | 77.56 |
| ATOM | 2842 | CB | LEU | A | 302 | 55.573 | 37.098 | 37.698 | 1.00 | 75.96 |
| ATOM | 2843 | CG | LEU | A | 302 | 55.833 | 36.472 | 36.337 | 1.00 | 75.33 |
| ATOM | 2844 | CD1 | LEU | A | 302 | 57.217 | 35.860 | 36.332 | 1.00 | 75.78 |
| ATOM | 2845 | CD2 | LEU | A | 302 | 54.767 | 35.425 | 36.070 | 1.00 | 74.63 |
| ATOM | 2846 | C | LEU | A | 302 | 53.782 | 37.667 | 39.338 | 1.00 | 80.50 |
| ATOM | 2847 | O | LEU | A | 302 | 54.567 | 37.228 | 40.173 | 1.00 | 81.63 |
| ATOM | 2848 | N | PRO | A | 303 | 52.551 | 38.075 | 39.678 | 1.00 | 82.47 |
| ATOM | 2849 | CD | PRO | A | 303 | 51.687 | 39.012 | 38.927 | 1.00 | 83.87 |
| ATOM | 2850 | CA | PRO | A | 303 | 52.127 | 38.002 | 41.087 | 1.00 | 82.85 |
| ATOM | 2851 | CB | PRO | A | 303 | 51.725 | 39.440 | 41.359 | 1.00 | 83.29 |
| ATOM | 2852 | CG | PRO | A | 303 | 50.963 | 39.803 | 40.046 | 1.00 | 84.24 |
| ATOM | 2853 | C | PRO | A | 303 | 50.924 | 37.070 | 41.346 | 1.00 | 82.81 |
| ATOM | 2854 | O | PRO | A | 303 | 49.809 | 37.403 | 40.953 | 1.00 | 84.47 |
| ATOM | 2855 | N | GLU | A | 304 | 51.087 | 35.893 | 41.943 | 1.00 | 81.68 |
| ATOM | 2857 | CA | GLU | A | 304 | 52.308 | 35.254 | 42.438 | 1.00 | 80.61 |
| ATOM | 2858 | CB | GLU | A | 304 | 52.767 | 34.203 | 41.437 | 1.00 | 83.61 |
| ATOM | 2859 | CG | GLU | A | 304 | 51.698 | 33.132 | 41.207 | 1.00 | 86.52 |
| ATOM | 2860 | CD | GLU | A | 304 | 51.179 | 32.502 | 42.499 | 1.00 | 88.36 |
| ATOM | 2861 | OE1 | GLU | A | 304 | 50.103 | 32.923 | 42.977 | 1.00 | 89.84 |
| ATOM | 2862 | OE2 | GLU | A | 304 | 51.847 | 31.594 | 43.036 | 1.00 | 89.41 |
| ATOM | 2863 | C | GLU | A | 304 | 53.501 | 35.887 | 43.175 | 1.00 | 78.52 |
| ATOM | 2864 | O | GLU | A | 304 | 53.383 | 36.142 | 44.361 | 1.00 | 78.12 |
| ATOM | 2865 | N | VAL | A | 305 | 54.817 | 36.106 | 42.616 | 1.00 | 76.19 |
| ATOM | 2867 | CA | VAL | A | 305 | 55.955 | 36.809 | 43.226 | 1.00 | 74.85 |
| ATOM | 2868 | CB | VAL | A | 305 | 56.904 | 37.447 | 42.170 | 1.00 | 74.41 |
| ATOM | 2869 | CG1 | VAL | A | 305 | 57.405 | 36.392 | 41.202 | 1.00 | 74.39 |
| ATOM | 2870 | CG2 | VAL | A | 305 | 56.218 | 38.578 | 41.433 | 1.00 | 75.20 |
| ATOM | 2871 | C | VAL | A | 305 | 55.622 | 37.833 | 44.316 | 1.00 | 74.68 |
| ATOM | 2872 | O | VAL | A | 305 | 56.110 | 37.711 | 45.438 | 1.00 | 75.57 |
| ATOM | 2873 | N | ALA | A | 306 | 54.746 | 38.790 | 44.011 | 1.00 | 73.53 |
| ATOM | 2875 | CA | ALA | A | 306 | 54.357 | 39.823 | 44.975 | 1.00 | 71.77 |
| ATOM | 2876 | CB | ALA | A | 306 | 53.893 | 41.068 | 44.248 | 1.00 | 71.20 |
| ATOM | 2877 | C | ALA | A | 306 | 53.273 | 39.348 | 45.935 | 1.00 | 71.50 |
| ATOM | 2878 | O | ALA | A | 306 | 53.087 | 39.924 | 47.005 | 1.00 | 70.71 |
| ATOM | 2879 | N | LYS | A | 307 | 52.563 | 38.300 | 45.529 | 1.00 | 71.78 |
| ATOM | 2881 | CA | LYS | A | 307 | 51.474 | 37.697 | 46.300 | 1.00 | 71.82 |
| ATOM | 2882 | CB | LYS | A | 307 | 50.508 | 37.010 | 45.324 | 1.00 | 72.68 |
| ATOM | 2883 | CG | LYS | A | 307 | 49.332 | 36.270 | 45.944 | 1.00 | 75.23 |
| ATOM | 2884 | CD | LYS | A | 307 | 48.473 | 35.638 | 44.848 | 1.00 | 76.97 |
| ATOM | 2885 | CE | LYS | A | 307 | 47.247 | 34.935 | 45.413 | 1.00 | 77.90 |
| ATOM | 2886 | NZ | LYS | A | 307 | 46.352 | 34.434 | 44.331 | 1.00 | 77.64 |
| ATOM | 2890 | C | LYS | A | 307 | 51.946 | 36.698 | 47.374 | 1.00 | 71.04 |
| ATOM | 2891 | O | LYS | A | 307 | 51.393 | 36.651 | 48.473 | 1.00 | 72.05 |
| ATOM | 2892 | N | LYS | A | 308 | 52.958 | 35.898 | 47.050 | 1.00 | 69.00 |
| ATOM | 2894 | CA | LYS | A | 308 | 53.480 | 34.903 | 47.979 | 1.00 | 66.59 |
| ATOM | 2895 | CB | LYS | A | 308 | 53.891 | 33.636 | 47.219 | 1.00 | 70.09 |
| ATOM | 2896 | CG | LYS | A | 308 | 53.887 | 32.346 | 48.046 | 1.00 | 75.24 |
| ATOM | 2897 | CD | LYS | A | 308 | 54.920 | 32.351 | 49.172 | 1.00 | 79.31 |
| ATOM | 2898 | CE | LYS | A | 308 | 54.842 | 31.091 | 50.027 | 1.00 | 81.43 |
| ATOM | 2899 | NZ | LYS | A | 308 | 55.760 | 31.141 | 51.206 | 1.00 | 82.05 |
| ATOM | 2903 | C | LYS | A | 308 | 54.680 | 35.467 | 48.722 | 1.00 | 64.17 |
| ATOM | 2904 | O | LYS | A | 308 | 54.840 | 35.240 | 49.921 | 1.00 | 65.45 |
| ATOM | 2905 | N | PHE | A | 309 | 55.526 | 36.200 | 48.007 | 1.00 | 60.99 |
| ATOM | 2907 | CA | PHE | A | 309 | 56.725 | 36.784 | 48.597 | 1.00 | 57.85 |
| ATOM | 2908 | CB | PHE | A | 309 | 57.974 | 36.274 | 47.853 | 1.00 | 53.07 |
| ATOM | 2909 | CG | PHE | A | 309 | 58.085 | 34.762 | 47.808 | 1.00 | 45.45 |
| ATOM | 2910 | CD1 | PHE | A | 309 | 58.551 | 34.050 | 48.906 | 1.00 | 42.52 |
| ATOM | 2911 | CD2 | PHE | A | 309 | 57.689 | 34.052 | 46.679 | 1.00 | 43.68 |
| ATOM | 2912 | CE1 | PHE | A | 309 | 58.619 | 32.655 | 48.883 | 1.00 | 38.70 |
| ATOM | 2913 | CE2 | PHE | A | 309 | 57.754 | 32.655 | 46.647 | 1.00 | 42.05 |
| ATOM | 2914 | CZ | PHE | A | 309 | 58.219 | 31.959 | 47.753 | 1.00 | 39.93 |
| ATOM | 2915 | C | PHE | A | 309 | 56.616 | 38.312 | 48.553 | 1.00 | 58.92 |
| ATOM | 2916 | O | PHE | A | 309 | 57.266 | 38.978 | 47.743 | 1.00 | 58.89 |
| ATOM | 2917 | N | PRO | A | 310 | 55.822 | 38.885 | 49.472 | 1.00 | 60.26 |
| ATOM | 2918 | CD | PRO | A | 310 | 55.173 | 38.142 | 50.569 | 1.00 | 61.34 |
| ATOM | 2919 | CA | PRO | A | 310 | 55.560 | 40.320 | 49.613 | 1.00 | 61.10 |
| ATOM | 2920 | CB | PRO | A | 310 | 54.522 | 40.358 | 50.736 | 1.00 | 62.64 |
| ATOM | 2921 | CG | PRO | A | 310 | 54.934 | 39.217 | 51.598 | 1.00 | 61.52 |
| ATOM | 2922 | C | PRO | A | 310 | 56.737 | 41.239 | 49.927 | 1.00 | 61.05 |
| ATOM | 2923 | O | PRO | A | 310 | 57.307 | 41.187 | 51.016 | 1.00 | 61.70 |
| ATOM | 2924 | N | ASN | A | 311 | 57.044 | 42.121 | 48.979 | 1.00 | 62.21 |
| ATOM | 2926 | CA | ASN | A | 311 | 58.116 | 43.111 | 49.111 | 1.00 | 63.11 |
| ATOM | 2927 | CB | ASN | A | 311 | 57.667 | 44.247 | 50.037 | 1.00 | 66.59 |
| ATOM | 2928 | CG | ASN | A | 311 | 58.589 | 45.448 | 49.976 | 1.00 | 69.63 |
| ATOM | 2929 | OD1 | ASN | A | 311 | 58.596 | 46.186 | 48.991 | 1.00 | 71.72 |
| ATOM | 2930 | ND2 | ASN | A | 311 | 59.373 | 45.651 | 51.027 | 1.00 | 71.51 |
| ATOM | 2933 | C | ASN | A | 311 | 59.458 | 42.547 | 49.583 | 1.00 | 61.81 |
| ATOM | 2934 | O | ASN | A | 311 | 59.855 | 42.737 | 50.738 | 1.00 | 63.25 |
| ATOM | 2935 | N | MET | A | 312 | 60.155 | 41.870 | 48.673 | 1.00 | 58.63 |
| ATOM | 2937 | CA | MET | A | 312 | 61.459 | 41.266 | 48.950 | 1.00 | 53.64 |
| ATOM | 2938 | CB | MET | A | 312 | 61.316 | 39.768 | 49.269 | 1.00 | 53.53 |
| ATOM | 2939 | CG | MET | A | 312 | 60.599 | 39.405 | 50.570 | 1.00 | 52.28 |
| ATOM | 2940 | SD | MET | A | 312 | 60.259 | 37.617 | 50.633 | 1.00 | 52.18 |
| ATOM | 2941 | CE | MET | A | 312 | 58.896 | 37.534 | 51.788 | 1.00 | 54.70 |
| ATOM | 2942 | C | MET | A | 312 | 62.320 | 41.411 | 47.699 | 1.00 | 49.72 |
| ATOM | 2943 | O | MET | A | 312 | 61.801 | 41.656 | 46.605 | 1.00 | 49.26 |
| ATOM | 2944 | N | LYS | A | 313 | 63.630 | 41.275 | 47.865 | 1.00 | 44.80 |
| ATOM | 2946 | CA | LYS | A | 313 | 64.549 | 41.363 | 46.741 | 1.00 | 41.81 |
| ATOM | 2947 | CB | LYS | A | 313 | 65.952 | 41.707 | 47.230 | 1.00 | 42.38 |
| ATOM | 2948 | CG | LYS | A | 313 | 66.050 | 43.081 | 47.846 | 1.00 | 47.04 |
| ATOM | 2949 | CD | LYS | A | 313 | 65.762 | 44.159 | 46.818 | 1.00 | 49.35 |
| ATOM | 2950 | CE | LYS | A | 313 | 65.599 | 45.515 | 47.481 | 1.00 | 53.10 |
| ATOM | 2951 | NZ | LYS | A | 313 | 66.805 | 45.897 | 48.268 | 1.00 | 56.75 |
| ATOM | 2955 | C | LYS | A | 313 | 64.562 | 40.018 | 46.026 | 1.00 | 39.61 |
| ATOM | 2956 | O | LYS | A | 313 | 64.133 | 39.013 | 46.592 | 1.00 | 38.93 |
| ATOM | 2957 | N | ILE | A | 314 | 65.040 | 40.005 | 44.786 | 1.00 | 37.30 |
| ATOM | 2959 | CA | ILE | A | 314 | 65.115 | 38.785 | 43.995 | 1.00 | 34.17 |
| ATOM | 2960 | CB | ILE | A | 314 | 64.224 | 38.865 | 42.716 | 1.00 | 33.93 |
| ATOM | 2961 | CG2 | ILE | A | 314 | 64.607 | 37.783 | 41.708 | 1.00 | 32.86 |
| ATOM | 2962 | CG1 | ILE | A | 314 | 62.745 | 38.728 | 43.093 | 1.00 | 33.03 |
| ATOM | 2963 | CD1 | ILE | A | 314 | 61.824 | 38.459 | 41.920 | 1.00 | 32.90 |
| ATOM | 2964 | C | ILE | A | 314 | 66.567 | 38.565 | 43.604 | 1.00 | 33.46 |
| ATOM | 2965 | O | ILE | A | 314 | 67.272 | 39.512 | 43.271 | 1.00 | 32.77 |
| ATOM | 2966 | N | GLN | A | 315 | 67.024 | 37.321 | 43.698 | 1.00 | 32.44 |
| ATOM | 2968 | CA | GLN | A | 315 | 68.391 | 36.975 | 43.336 | 1.00 | 32.66 |
| ATOM | 2969 | CB | GLN | A | 315 | 69.118 | 36.366 | 44.529 | 1.00 | 34.35 |
| ATOM | 2970 | CG | GLN | A | 315 | 70.624 | 36.305 | 44.376 | 1.00 | 35.08 |
| ATOM | 2971 | CD | GLN | A | 315 | 71.317 | 35.839 | 45.639 | 1.00 | 33.95 |
| ATOM | 2972 | OE1 | GLN | A | 315 | 71.579 | 36.620 | 46.548 | 1.00 | 34.44 |
| ATOM | 2973 | NE2 | GLN | A | 315 | 71.607 | 34.554 | 45.701 | 1.00 | 37.53 |
| ATOM | 2976 | C | GLN | A | 315 | 68.307 | 35.964 | 42.207 | 1.00 | 33.04 |
| ATOM | 2977 | O | GLN | A | 315 | 67.520 | 35.024 | 42.271 | 1.00 | 35.05 |
| ATOM | 2978 | N | ILE | A | 316 | 69.116 | 36.155 | 41.175 | 1.00 | 34.76 |
| ATOM | 2980 | CA | ILE | A | 316 | 69.106 | 35.268 | 40.018 | 1.00 | 33.81 |
| ATOM | 2981 | CB | ILE | A | 316 | 68.711 | 36.067 | 38.750 | 1.00 | 35.72 |
| ATOM | 2982 | CG2 | ILE | A | 316 | 68.944 | 35.247 | 37.498 | 1.00 | 37.16 |
| ATOM | 2983 | CG1 | ILE | A | 316 | 67.249 | 36.518 | 38.864 | 1.00 | 36.98 |
| ATOM | 2984 | CD1 | ILE | A | 316 | 66.695 | 37.179 | 37.633 | 1.00 | 38.34 |
| ATOM | 2985 | C | ILE | A | 316 | 70.445 | 34.548 | 39.823 | 1.00 | 32.92 |
| ATOM | 2986 | O | ILE | A | 316 | 71.513 | 35.160 | 39.900 | 1.00 | 33.03 |
| ATOM | 2987 | N | HIS | A | 317 | 70.386 | 33.238 | 39.609 | 1.00 | 33.55 |
| ATOM | 2989 | CA | HIS | A | 317 | 71.586 | 32.428 | 39.409 | 1.00 | 32.00 |
| ATOM | 2990 | CB | HIS | A | 317 | 71.532 | 31.162 | 40.264 | 1.00 | 32.50 |
| ATOM | 2991 | CG | HIS | A | 317 | 71.659 | 31.411 | 41.735 | 1.00 | 32.14 |
| ATOM | 2992 | CD2 | HIS | A | 317 | 71.889 | 32.548 | 42.435 | 1.00 | 31.63 |
| ATOM | 2993 | ND1 | HIS | A | 317 | 71.572 | 30.398 | 42.666 | 1.00 | 32.81 |
| ATOM | 2995 | CE1 | HIS | A | 317 | 71.746 | 30.899 | 43.875 | 1.00 | 33.94 |
| ATOM | 2996 | NE2 | HIS | A | 317 | 71.941 | 32.201 | 43.761 | 1.00 | 32.58 |
| ATOM | 2998 | C | HIS | A | 317 | 71.720 | 32.014 | 37.953 | 1.00 | 33.04 |
| ATOM | 2999 | O | HIS | A | 317 | 70.844 | 31.335 | 37.416 | 1.00 | 33.39 |
| ATOM | 3000 | N | VAL | A | 318 | 72.842 | 32.377 | 37.344 | 1.00 | 33.42 |
| ATOM | 3002 | CA | VAL | A | 318 | 73.129 | 32.059 | 35.952 | 1.00 | 31.61 |
| ATOM | 3003 | CB | VAL | A | 318 | 73.535 | 33.338 | 35.193 | 1.00 | 29.43 |
| ATOM | 3004 | CG1 | VAL | A | 318 | 73.993 | 33.003 | 33.789 | 1.00 | 30.90 |
| ATOM | 3005 | CG2 | VAL | A | 318 | 72.376 | 34.311 | 35.161 | 1.00 | 27.51 |
| ATOM | 3006 | C | VAL | A | 318 | 74.273 | 31.043 | 35.861 | 1.00 | 32.81 |
| ATOM | 3007 | O | VAL | A | 318 | 75.392 | 31.325 | 36.296 | 1.00 | 33.30 |
| ATOM | 3008 | N | SER | A | 319 | 73.990 | 29.858 | 35.329 | 1.00 | 32.14 |
| ATOM | 3010 | CA | SER | A | 319 | 75.021 | 28.836 | 35.175 | 1.00 | 32.12 |
| ATOM | 3011 | CB | SER | A | 319 | 74.919 | 27.778 | 36.271 | 1.00 | 28.41 |
| ATOM | 3012 | OG | SER | A | 319 | 73.723 | 27.039 | 36.158 | 1.00 | 29.90 |
| ATOM | 3014 | C | SER | A | 319 | 74.900 | 28.170 | 33.815 | 1.00 | 33.52 |
| ATOM | 3015 | O | SER | A | 319 | 73.834 | 28.174 | 33.210 | 1.00 | 37.50 |
| ATOM | 3016 | N | ALA | A | 320 | 76.005 | 27.632 | 33.319 | 1.00 | 35.33 |
| ATOM | 3018 | CA | ALA | A | 320 | 76.009 | 26.958 | 32.031 | 1.00 | 35.02 |
| ATOM | 3019 | CB | ALA | A | 320 | 77.417 | 26.914 | 31.461 | 1.00 | 36.41 |
| ATOM | 3020 | C | ALA | A | 320 | 75.466 | 25.547 | 32.210 | 1.00 | 36.23 |
| ATOM | 3021 | O | ALA | A | 320 | 75.955 | 24.786 | 33.047 | 1.00 | 35.14 |
| ATOM | 3022 | N | SER | A | 321 | 74.459 | 25.204 | 31.415 | 1.00 | 38.15 |
| ATOM | 3024 | CA | SER | A | 321 | 73.835 | 23.891 | 31.478 | 1.00 | 38.99 |
| ATOM | 3025 | CB | SER | A | 321 | 72.472 | 23.935 | 30.786 | 1.00 | 38.78 |
| ATOM | 3026 | OG | SER | A | 321 | 72.418 | 24.984 | 29.834 | 1.00 | 40.20 |
| ATOM | 3028 | C | SER | A | 321 | 74.724 | 22.824 | 30.848 | 1.00 | 40.78 |
| ATOM | 3029 | O | SER | A | 321 | 74.942 | 21.760 | 31.430 | 1.00 | 44.24 |
| ATOM | 3030 | N | THR | A | 322 | 75.249 | 23.126 | 29.666 | 1.00 | 39.66 |
| ATOM | 3032 | CA | THR | A | 322 | 76.119 | 22.214 | 28.939 | 1.00 | 36.30 |
| ATOM | 3033 | CB | THR | A | 322 | 75.355 | 21.553 | 27.750 | 1.00 | 39.80 |
| ATOM | 3034 | OG1 | THR | A | 322 | 74.613 | 22.548 | 27.027 | 1.00 | 40.24 |
| ATOM | 3036 | CG2 | THR | A | 322 | 74.398 | 20.473 | 28.253 | 1.00 | 41.99 |
| ATOM | 3037 | C | THR | A | 322 | 77.332 | 22.985 | 28.416 | 1.00 | 33.41 |
| ATOM | 3038 | O | THR | A | 322 | 77.372 | 24.214 | 28.487 | 1.00 | 31.80 |
| ATOM | 3039 | N | PRO | A | 323 | 78.378 | 22.272 | 27.966 | 1.00 | 32.97 |
| ATOM | 3040 | CD | PRO | A | 323 | 78.606 | 20.826 | 28.133 | 1.00 | 31.42 |
| ATOM | 3041 | CA | PRO | A | 323 | 79.581 | 22.925 | 27.439 | 1.00 | 31.79 |
| ATOM | 3042 | CB | PRO | A | 323 | 80.471 | 21.739 | 27.089 | 1.00 | 31.21 |
| ATOM | 3043 | CG | PRO | A | 323 | 80.099 | 20.740 | 28.128 | 1.00 | 31.27 |
| ATOM | 3044 | C | PRO | A | 323 | 79.279 | 23.757 | 26.191 | 1.00 | 33.72 |
| ATOM | 3045 | O | PRO | A | 323 | 78.727 | 23.237 | 25.218 | 1.00 | 35.68 |
| ATOM | 3046 | N | PRO | A | 324 | 79.650 | 25.053 | 26.196 | 1.00 | 34.41 |
| ATOM | 3047 | CD | PRO | A | 324 | 80.303 | 25.799 | 27.286 | 1.00 | 34.36 |
| ATOM | 3048 | CA | PRO | A | 324 | 79.407 | 25.933 | 25.050 | 1.00 | 36.18 |
| ATOM | 3049 | CB | PRO | A | 324 | 79.863 | 27.301 | 25.567 | 1.00 | 34.56 |
| ATOM | 3050 | CG | PRO | A | 324 | 80.913 | 26.963 | 26.551 | 1.00 | 34.54 |
| ATOM | 3051 | C | PRO | A | 324 | 80.186 | 25.511 | 23.811 | 1.00 | 39.38 |
| ATOM | 3052 | O | PRO | A | 324 | 81.397 | 25.278 | 23.871 | 1.00 | 43.04 |
| ATOM | 3053 | N | HIS | A | 325 | 79.468 | 25.364 | 22.704 | 1.00 | 39.27 |
| ATOM | 3055 | CA | HIS | A | 325 | 80.064 | 24.973 | 21.434 | 1.00 | 39.40 |
| ATOM | 3056 | CB | HIS | A | 325 | 79.045 | 24.236 | 20.562 | 1.00 | 44.72 |
| ATOM | 3057 | CG | HIS | A | 325 | 78.817 | 22.813 | 20.955 | 1.00 | 49.32 |
| ATOM | 3058 | CD2 | HIS | A | 325 | 79.471 | 22.015 | 21.831 | 1.00 | 51.91 |
| ATOM | 3059 | ND1 | HIS | A | 325 | 77.817 | 22.041 | 20.405 | 1.00 | 52.21 |
| ATOM | 3061 | CE1 | HIS | A | 325 | 77.864 | 20.827 | 20.924 | 1.00 | 53.83 |
| ATOM | 3062 | NE2 | HIS | A | 325 | 78.859 | 20.785 | 21.793 | 1.00 | 54.25 |
| ATOM | 3064 | C | HIS | A | 325 | 80.537 | 26.183 | 20.658 | 1.00 | 37.89 |
| ATOM | 3065 | O | HIS | A | 325 | 80.011 | 27.285 | 20.825 | 1.00 | 39.24 |
| ATOM | 3066 | N | LEU | A | 326 | 81.514 | 25.958 | 19.788 | 1.00 | 36.12 |
| ATOM | 3068 | CA | LEU | A | 326 | 82.056 | 26.993 | 18.928 | 1.00 | 34.45 |
| ATOM | 3069 | CB | LEU | A | 326 | 83.370 | 27.535 | 19.487 | 1.00 | 34.16 |
| ATOM | 3070 | CG | LEU | A | 326 | 83.906 | 28.758 | 18.745 | 1.00 | 34.49 |
| ATOM | 3071 | CD1 | LEU | A | 326 | 82.912 | 29.878 | 18.854 | 1.00 | 36.18 |
| ATOM | 3072 | CD2 | LEU | A | 326 | 85.220 | 29.194 | 19.321 | 1.00 | 38.20 |
| ATOM | 3073 | C | LEU | A | 326 | 82.285 | 26.329 | 17.569 | 1.00 | 34.87 |
| ATOM | 3074 | O | LEU | A | 326 | 82.981 | 25.311 | 17.469 | 1.00 | 35.31 |
| ATOM | 3075 | N | SER | A | 327 | 81.639 | 26.863 | 16.542 | 1.00 | 32.90 |
| ATOM | 3077 | CA | SER | A | 327 | 81.755 | 26.323 | 15.202 | 1.00 | 32.76 |
| ATOM | 3078 | CB | SER | A | 327 | 80.359 | 26.173 | 14.591 | 1.00 | 34.44 |
| ATOM | 3079 | OG | SER | A | 327 | 80.390 | 25.407 | 13.394 | 1.00 | 41.05 |
| ATOM | 3081 | C | SER | A | 327 | 82.607 | 27.247 | 14.343 | 1.00 | 30.57 |
| ATOM | 3082 | O | SER | A | 327 | 82.391 | 28.458 | 14.329 | 1.00 | 31.41 |
| ATOM | 3083 | N | VAL | A | 328 | 83.601 | 26.691 | 13.658 | 1.00 | 29.84 |
| ATOM | 3085 | CA | VAL | A | 328 | 84.452 | 27.502 | 12.795 | 1.00 | 28.60 |
| ATOM | 3086 | CB | VAL | A | 328 | 85.951 | 27.275 | 13.056 | 1.00 | 25.61 |
| ATOM | 3087 | CG1 | VAL | A | 328 | 86.733 | 28.389 | 12.423 | 1.00 | 24.98 |
| ATOM | 3088 | CG2 | VAL | A | 328 | 86.245 | 27.218 | 14.544 | 1.00 | 23.23 |
| ATOM | 3089 | C | VAL | A | 328 | 84.159 | 27.174 | 11.336 | 1.00 | 30.19 |
| ATOM | 3090 | O | VAL | A | 328 | 84.221 | 26.015 | 10.928 | 1.00 | 29.56 |
| ATOM | 3091 | N | GLN | A | 329 | 83.791 | 28.194 | 10.569 | 1.00 | 32.32 |
| ATOM | 3093 | CA | GLN | A | 329 | 83.479 | 28.038 | 9.150 | 1.00 | 34.28 |
| ATOM | 3094 | CB | GLN | A | 329 | 81.956 | 27.972 | 8.939 | 1.00 | 39.33 |
| ATOM | 3095 | CG | GLN | A | 329 | 81.222 | 26.924 | 9.774 | 1.00 | 48.55 |
| ATOM | 3096 | CD | GLN | A | 329 | 80.246 | 27.532 | 10.785 | 1.00 | 53.31 |
| ATOM | 3097 | OE1 | GLN | A | 329 | 79.321 | 26.860 | 11.245 | 1.00 | 57.70 |
| ATOM | 3098 | NE2 | GLN | A | 329 | 80.456 | 28.796 | 11.142 | 1.00 | 53.89 |
| ATOM | 3101 | C | GLN | A | 329 | 84.050 | 29.259 | 8.416 | 1.00 | 32.74 |
| ATOM | 3102 | O | GLN | A | 329 | 84.337 | 30.279 | 9.048 | 1.00 | 34.32 |
| ATOM | 3103 | N | PRO | A | 330 | 84.176 | 29.193 | 7.073 | 1.00 | 30.85 |
| ATOM | 3104 | CD | PRO | A | 330 | 83.896 | 28.019 | 6.226 | 1.00 | 27.63 |
| ATOM | 3105 | CA | PRO | A | 330 | 84.711 | 30.301 | 6.265 | 1.00 | 30.72 |
| ATOM | 3106 | CB | PRO | A | 330 | 84.597 | 29.765 | 4.842 | 1.00 | 27.09 |
| ATOM | 3107 | CG | PRO | A | 330 | 84.754 | 28.286 | 5.026 | 1.00 | 27.10 |
| ATOM | 3108 | C | PRO | A | 330 | 83.968 | 31.635 | 6.416 | 1.00 | 33.05 |
| ATOM | 3109 | O | PRO | A | 330 | 84.501 | 32.695 | 6.084 | 1.00 | 34.38 |
| ATOM | 3110 | N | THR | A | 331 | 82.733 | 31.575 | 6.904 | 1.00 | 36.91 |
| ATOM | 3112 | CA | THR | A | 331 | 81.918 | 32.767 | 7.106 | 1.00 | 39.55 |
| ATOM | 3113 | CB | THR | A | 331 | 80.414 | 32.414 | 7.065 | 1.00 | 41.23 |
| ATOM | 3114 | OG1 | THR | A | 331 | 80.179 | 31.221 | 7.826 | 1.00 | 42.58 |
| ATOM | 3116 | CG2 | THR | A | 331 | 79.959 | 32.180 | 5.630 | 1.00 | 42.18 |
| ATOM | 3117 | C | THR | A | 331 | 82.244 | 33.469 | 8.425 | 1.00 | 41.21 |
| ATOM | 3118 | O | THR | A | 331 | 82.114 | 34.691 | 8.535 | 1.00 | 44.22 |
| ATOM | 3119 | N | GLY | A | 332 | 82.671 | 32.691 | 9.418 | 1.00 | 39.90 |
| ATOM | 3121 | CA | GLY | A | 332 | 83.005 | 33.240 | 10.720 | 1.00 | 36.14 |
| ATOM | 3122 | C | GLY | A | 332 | 82.788 | 32.188 | 11.788 | 1.00 | 36.29 |
| ATOM | 3123 | O | GLY | A | 332 | 82.399 | 31.058 | 11.472 | 1.00 | 36.21 |
| ATOM | 3124 | N | LEU | A | 333 | 83.048 | 32.542 | 13.044 | 1.00 | 35.70 |
| ATOM | 3126 | CA | LEU | A | 333 | 82.875 | 31.606 | 14.153 | 1.00 | 34.12 |
| ATOM | 3127 | CB | LEU | A | 333 | 84.037 | 31.704 | 15.157 | 1.00 | 31.15 |
| ATOM | 3128 | CG | LEU | A | 333 | 85.438 | 32.183 | 14.766 | 1.00 | 30.79 |
| ATOM | 3129 | CD1 | LEU | A | 333 | 86.402 | 31.841 | 15.886 | 1.00 | 29.93 |
| ATOM | 3130 | CD2 | LEU | A | 333 | 85.908 | 31.539 | 13.492 | 1.00 | 32.11 |
| ATOM | 3131 | C | LEU | A | 333 | 81.570 | 31.889 | 14.889 | 1.00 | 34.02 |
| ATOM | 3132 | O | LEU | A | 333 | 81.283 | 33.037 | 15.233 | 1.00 | 35.28 |
| ATOM | 3133 | N | THR | A | 334 | 80.777 | 30.850 | 15.123 | 1.00 | 32.89 |
| ATOM | 3135 | CA | THR | A | 334 | 79.521 | 30.997 | 15.842 | 1.00 | 30.97 |
| ATOM | 3136 | CB | THR | A | 334 | 78.344 | 30.435 | 15.037 | 1.00 | 30.40 |
| ATOM | 3137 | OG1 | THR | A | 334 | 78.673 | 29.124 | 14.568 | 1.00 | 36.09 |
| ATOM | 3139 | CG2 | THR | A | 334 | 78.043 | 31.324 | 13.850 | 1.00 | 27.87 |
| ATOM | 3140 | C | THR | A | 334 | 79.649 | 30.274 | 17.184 | 1.00 | 30.59 |
| ATOM | 3141 | O | THR | A | 334 | 80.293 | 29.229 | 17.267 | 1.00 | 31.11 |
| ATOM | 3142 | N | PHE | A | 335 | 79.052 | 30.853 | 18.225 | 1.00 | 31.55 |
| ATOM | 3144 | CA | PHE | A | 335 | 79.095 | 30.334 | 19.598 | 1.00 | 30.95 |
| ATOM | 3145 | CB | PHE | A | 335 | 79.579 | 31.465 | 20.530 | 1.00 | 31.73 |
| ATOM | 3146 | CG | PHE | A | 335 | 80.138 | 31.001 | 21.853 | 1.00 | 32.43 |
| ATOM | 3147 | CD1 | PHE | A | 335 | 81.224 | 30.134 | 21.907 | 1.00 | 34.10 |
| ATOM | 3148 | CD2 | PHE | A | 335 | 79.611 | 31.483 | 23.044 | 1.00 | 33.32 |
| ATOM | 3149 | CE1 | PHE | A | 335 | 81.779 | 29.755 | 23.124 | 1.00 | 36.27 |
| ATOM | 3150 | CE2 | PHE | A | 335 | 80.156 | 31.113 | 24.267 | 1.00 | 36.93 |
| ATOM | 3151 | CZ | PHE | A | 335 | 81.243 | 30.248 | 24.308 | 1.00 | 36.83 |
| ATOM | 3152 | C | PHE | A | 335 | 77.674 | 29.910 | 19.975 | 1.00 | 29.88 |
| ATOM | 3153 | O | PHE | A | 335 | 76.722 | 30.616 | 19.666 | 1.00 | 29.27 |
| ATOM | 3154 | N | TYR | A | 336 | 77.533 | 28.779 | 20.658 | 1.00 | 30.87 |
| ATOM | 3156 | CA | TYR | A | 336 | 76.216 | 28.281 | 21.052 | 1.00 | 33.87 |
| ATOM | 3157 | CB | TYR | A | 336 | 75.936 | 26.953 | 20.345 | 1.00 | 35.86 |
| ATOM | 3158 | CG | TYR | A | 336 | 76.081 | 27.036 | 18.844 | 1.00 | 36.58 |
| ATOM | 3159 | CD1 | TYR | A | 336 | 75.107 | 27.665 | 18.072 | 1.00 | 38.45 |
| ATOM | 3160 | CE1 | TYR | A | 336 | 75.245 | 27.791 | 16.700 | 1.00 | 38.32 |
| ATOM | 3161 | CD2 | TYR | A | 336 | 77.207 | 26.526 | 18.198 | 1.00 | 34.97 |
| ATOM | 3162 | CE2 | TYR | A | 336 | 77.357 | 26.647 | 16.819 | 1.00 | 36.42 |
| ATOM | 3163 | CZ | TYR | A | 336 | 76.369 | 27.285 | 16.078 | 1.00 | 37.51 |
| ATOM | 3164 | OH | TYR | A | 336 | 76.494 | 27.435 | 14.715 | 1.00 | 41.83 |
| ATOM | 3166 | C | TYR | A | 336 | 76.104 | 28.096 | 22.565 | 1.00 | 36.66 |
| ATOM | 3167 | O | TYR | A | 336 | 76.280 | 26.988 | 23.074 | 1.00 | 39.11 |
| ATOM | 3168 | N | PRO | A | 337 | 75.858 | 29.187 | 23.309 | 1.00 | 36.71 |
| ATOM | 3169 | CD | PRO | A | 337 | 75.890 | 30.605 | 22.904 | 1.00 | 36.11 |
| ATOM | 3170 | CA | PRO | A | 337 | 75.740 | 29.069 | 24.761 | 1.00 | 35.26 |
| ATOM | 3171 | CB | PRO | A | 337 | 76.156 | 30.449 | 25.234 | 1.00 | 35.76 |
| ATOM | 3172 | CG | PRO | A | 337 | 75.564 | 31.327 | 24.192 | 1.00 | 36.30 |
| ATOM | 3173 | C | PRO | A | 337 | 74.325 | 28.751 | 25.198 | 1.00 | 36.60 |
| ATOM | 3174 | O | PRO | A | 337 | 73.358 | 29.231 | 24.604 | 1.00 | 39.83 |
| ATOM | 3175 | N | ALA | A | 338 | 74.218 | 27.909 | 26.218 | 1.00 | 36.08 |
| ATOM | 3177 | CA | ALA | A | 338 | 72.944 | 27.512 | 26.802 | 1.00 | 35.56 |
| ATOM | 3178 | CB | ALA | A | 338 | 72.671 | 26.039 | 26.533 | 1.00 | 36.41 |
| ATOM | 3179 | C | ALA | A | 338 | 73.116 | 27.752 | 28.294 | 1.00 | 36.55 |
| ATOM | 3180 | O | ALA | A | 338 | 74.059 | 27.241 | 28.902 | 1.00 | 37.65 |
| ATOM | 3181 | N | VAL | A | 339 | 72.256 | 28.576 | 28.878 | 1.00 | 36.13 |
| ATOM | 3183 | CA | VAL | A | 339 | 72.360 | 28.872 | 30.295 | 1.00 | 34.95 |
| ATOM | 3184 | CB | VAL | A | 339 | 72.771 | 30.346 | 30.538 | 1.00 | 33.81 |
| ATOM | 3185 | CG1 | VAL | A | 339 | 74.046 | 30.667 | 29.776 | 1.00 | 36.08 |
| ATOM | 3186 | CG2 | VAL | A | 339 | 71.658 | 31.293 | 30.139 | 1.00 | 33.93 |
| ATOM | 3187 | C | VAL | A | 339 | 71.071 | 28.578 | 31.043 | 1.00 | 35.50 |
| ATOM | 3188 | O | VAL | A | 339 | 69.990 | 28.553 | 30.451 | 1.00 | 38.45 |
| ATOM | 3189 | N | ASP | A | 340 | 71.208 | 28.295 | 32.335 | 1.00 | 33.66 |
| ATOM | 3191 | CA | ASP | A | 340 | 70.078 | 28.029 | 33.213 | 1.00 | 31.12 |
| ATOM | 3192 | CB | ASP | A | 340 | 70.318 | 26.785 | 34.067 | 1.00 | 31.54 |
| ATOM | 3193 | CG | ASP | A | 340 | 69.813 | 25.520 | 33.411 | 1.00 | 33.33 |
| ATOM | 3194 | OD1 | ASP | A | 340 | 68.895 | 25.606 | 32.569 | 1.00 | 35.59 |
| ATOM | 3195 | OD2 | ASP | A | 340 | 70.324 | 24.432 | 33.742 | 1.00 | 35.60 |
| ATOM | 3196 | C | ASP | A | 340 | 70.003 | 29.235 | 34.108 | 1.00 | 29.12 |
| ATOM | 3197 | O | ASP | A | 340 | 70.968 | 29.555 | 34.794 | 1.00 | 29.84 |
| ATOM | 3198 | N | VAL | A | 341 | 68.890 | 29.948 | 34.040 | 1.00 | 28.41 |
| ATOM | 3200 | CA | VAL | A | 341 | 68.687 | 31.133 | 34.846 | 1.00 | 28.82 |
| ATOM | 3201 | CB | VAL | A | 341 | 68.249 | 32.316 | 33.969 | 1.00 | 29.73 |
| ATOM | 3202 | CG1 | VAL | A | 341 | 67.868 | 33.496 | 34.831 | 1.00 | 27.89 |
| ATOM | 3203 | CG2 | VAL | A | 341 | 69.364 | 32.703 | 33.017 | 1.00 | 28.62 |
| ATOM | 3204 | C | VAL | A | 341 | 67.592 | 30.833 | 35.852 | 1.00 | 30.90 |
| ATOM | 3205 | O | VAL | A | 341 | 66.456 | 30.596 | 35.459 | 1.00 | 34.21 |
| ATOM | 3206 | N | GLN | A | 342 | 67.931 | 30.821 | 37.139 | 1.00 | 31.91 |
| ATOM | 3208 | CA | GLN | A | 342 | 66.948 | 30.546 | 38.191 | 1.00 | 31.19 |
| ATOM | 3209 | CB | GLN | A | 342 | 67.375 | 29.345 | 39.039 | 1.00 | 31.37 |
| ATOM | 3210 | CG | GLN | A | 342 | 66.319 | 28.867 | 40.028 | 1.00 | 31.56 |
| ATOM | 3211 | CD | GLN | A | 342 | 66.706 | 27.570 | 40.706 | 1.00 | 33.87 |
| ATOM | 3212 | OE1 | GLN | A | 342 | 67.841 | 27.111 | 40.590 | 1.00 | 37.83 |
| ATOM | 3213 | NE2 | GLN | A | 342 | 65.770 | 26.979 | 41.431 | 1.00 | 35.30 |
| ATOM | 3216 | C | GLN | A | 342 | 66.752 | 31.761 | 39.088 | 1.00 | 31.20 |
| ATOM | 3217 | O | GLN | A | 342 | 67.724 | 32.374 | 39.537 | 1.00 | 31.67 |
| ATOM | 3218 | N | ALA | A | 343 | 65.494 | 32.114 | 39.327 | 1.00 | 30.96 |
| ATOM | 3220 | CA | ALA | A | 343 | 65.169 | 33.254 | 40.169 | 1.00 | 28.72 |
| ATOM | 3221 | CB | ALA | A | 343 | 64.042 | 34.054 | 39.563 | 1.00 | 28.77 |
| ATOM | 3222 | C | ALA | A | 343 | 64.782 | 32.778 | 41.552 | 1.00 | 28.72 |
| ATOM | 3223 | O | ALA | A | 343 | 64.011 | 31.826 | 41.700 | 1.00 | 30.49 |
| ATOM | 3224 | N | PHE | A | 344 | 65.320 | 33.447 | 42.559 | 1.00 | 26.88 |
| ATOM | 3226 | CA | PHE | A | 344 | 65.040 | 33.125 | 43.945 | 1.00 | 26.71 |
| ATOM | 3227 | CB | PHE | A | 344 | 66.316 | 32.718 | 44.673 | 1.00 | 21.35 |
| ATOM | 3228 | CG | PHE | A | 344 | 66.891 | 31.428 | 44.210 | 1.00 | 19.20 |
| ATOM | 3229 | CD1 | PHE | A | 344 | 67.806 | 31.396 | 43.173 | 1.00 | 20.33 |
| ATOM | 3230 | CD2 | PHE | A | 344 | 66.547 | 30.247 | 44.834 | 1.00 | 15.57 |
| ATOM | 3231 | CE1 | PHE | A | 344 | 68.372 | 30.198 | 42.766 | 1.00 | 19.57 |
| ATOM | 3232 | CE2 | PHE | A | 344 | 67.106 | 29.049 | 44.438 | 1.00 | 17.79 |
| ATOM | 3233 | CZ | PHE | A | 344 | 68.023 | 29.026 | 43.401 | 1.00 | 19.23 |
| ATOM | 3234 | C | PHE | A | 344 | 64.523 | 34.382 | 44.608 | 1.00 | 29.77 |
| ATOM | 3235 | O | PHE | A | 344 | 64.847 | 35.495 | 44.189 | 1.00 | 33.20 |
| ATOM | 3236 | N | ALA | A | 345 | 63.712 | 34.206 | 45.637 | 1.00 | 29.97 |
| ATOM | 3238 | CA | ALA | A | 345 | 63.204 | 35.331 | 46.390 | 1.00 | 30.90 |
| ATOM | 3239 | CB | ALA | A | 345 | 61.720 | 35.147 | 46.685 | 1.00 | 32.55 |
| ATOM | 3240 | C | ALA | A | 345 | 64.030 | 35.266 | 47.669 | 1.00 | 31.52 |
| ATOM | 3241 | O | ALA | A | 345 | 64.230 | 34.186 | 48.220 | 1.00 | 32.91 |
| ATOM | 3242 | N | VAL | A | 346 | 64.588 | 36.395 | 48.082 | 1.00 | 32.21 |
| ATOM | 3244 | CA | VAL | A | 346 | 65.403 | 36.449 | 49.287 | 1.00 | 33.87 |
| ATOM | 3245 | CB | VAL | A | 346 | 66.490 | 37.559 | 49.176 | 1.00 | 34.53 |
| ATOM | 3246 | CG1 | VAL | A | 346 | 67.430 | 37.537 | 50.383 | 1.00 | 32.13 |
| ATOM | 3247 | CG2 | VAL | A | 346 | 67.276 | 37.394 | 47.892 | 1.00 | 33.38 |
| ATOM | 3248 | C | VAL | A | 346 | 64.497 | 36.755 | 50.474 | 1.00 | 36.21 |
| ATOM | 3249 | O | VAL | A | 346 | 63.985 | 37.871 | 50.597 | 1.00 | 38.00 |
| ATOM | 3250 | N | LEU | A | 347 | 64.278 | 35.759 | 51.327 | 1.00 | 37.36 |
| ATOM | 3252 | CA | LEU | A | 347 | 63.445 | 35.928 | 52.516 | 1.00 | 39.35 |
| ATOM | 3253 | CB | LEU | A | 347 | 63.172 | 34.567 | 53.162 | 1.00 | 37.87 |
| ATOM | 3254 | CG | LEU | A | 347 | 62.664 | 33.460 | 52.238 | 1.00 | 37.83 |
| ATOM | 3255 | CD1 | LEU | A | 347 | 62.315 | 32.231 | 53.057 | 1.00 | 36.57 |
| ATOM | 3256 | CD2 | LEU | A | 347 | 61.461 | 33.945 | 51.462 | 1.00 | 39.01 |
| ATOM | 3257 | C | LEU | A | 347 | 64.157 | 36.847 | 53.519 | 1.00 | 42.26 |
| ATOM | 3258 | O | LEU | A | 347 | 65.376 | 37.021 | 53.449 | 1.00 | 44.26 |
| ATOM | 3259 | N | PRO | A | 348 | 63.418 | 37.410 | 54.495 | 1.00 | 43.97 |
| ATOM | 3260 | CD | PRO | A | 348 | 61.973 | 37.254 | 54.728 | 1.00 | 43.89 |
| ATOM | 3261 | CA | PRO | A | 348 | 64.002 | 38.309 | 55.505 | 1.00 | 43.61 |
| ATOM | 3262 | CB | PRO | A | 348 | 62.796 | 38.689 | 56.364 | 1.00 | 44.62 |
| ATOM | 3263 | CG | PRO | A | 348 | 61.875 | 37.514 | 56.206 | 1.00 | 44.98 |
| ATOM | 3264 | C | PRO | A | 348 | 65.138 | 37.751 | 56.364 | 1.00 | 42.78 |
| ATOM | 3265 | O | PRO | A | 348 | 65.685 | 38.471 | 57.195 | 1.00 | 43.70 |
| ATOM | 3266 | N | ASN | A | 349 | 65.478 | 36.478 | 56.184 | 1.00 | 42.71 |
| ATOM | 3268 | CA | ASN | A | 349 | 66.556 | 35.849 | 56.947 | 1.00 | 41.55 |
| ATOM | 3269 | CB | ASN | A | 349 | 66.046 | 34.591 | 57.659 | 1.00 | 41.16 |
| ATOM | 3270 | CG | ASN | A | 349 | 65.583 | 33.518 | 56.689 | 1.00 | 43.53 |
| ATOM | 3271 | OD1 | ASN | A | 349 | 65.524 | 33.748 | 55.479 | 1.00 | 44.53 |
| ATOM | 3272 | ND2 | ASN | A | 349 | 65.256 | 32.339 | 57.210 | 1.00 | 43.58 |
| ATOM | 3275 | C | ASN | A | 349 | 67.731 | 35.482 | 56.038 | 1.00 | 41.09 |
| ATOM | 3276 | O | ASN | A | 349 | 68.662 | 34.801 | 56.469 | 1.00 | 41.04 |
| ATOM | 3277 | N | SER | A | 350 | 67.678 | 35.954 | 54.793 | 1.00 | 40.23 |
| ATOM | 3279 | CA | SER | A | 350 | 68.697 | 35.704 | 53.768 | 1.00 | 39.62 |
| ATOM | 3280 | CB | SER | A | 350 | 70.122 | 35.854 | 54.314 | 1.00 | 40.62 |
| ATOM | 3281 | OG | SER | A | 350 | 70.518 | 37.214 | 54.317 | 1.00 | 46.79 |
| ATOM | 3283 | C | SER | A | 350 | 68.561 | 34.364 | 53.067 | 1.00 | 37.74 |
| ATOM | 3284 | O | SER | A | 350 | 69.339 | 34.063 | 52.168 | 1.00 | 40.17 |
| ATOM | 3285 | N | ALA | A | 351 | 67.581 | 33.562 | 53.471 | 1.00 | 34.00 |
| ATOM | 3287 | CA | ALA | A | 351 | 67.373 | 32.266 | 52.846 | 1.00 | 31.70 |
| ATOM | 3288 | CB | ALA | A | 351 | 66.426 | 31.429 | 53.670 | 1.00 | 31.56 |
| ATOM | 3289 | C | ALA | A | 351 | 66.799 | 32.505 | 51.455 | 1.00 | 32.27 |
| ATOM | 3290 | O | ALA | A | 351 | 66.150 | 33.526 | 51.212 | 1.00 | 34.50 |
| ATOM | 3291 | N | LEU | A | 352 | 67.022 | 31.560 | 50.549 | 1.00 | 30.81 |
| ATOM | 3293 | CA | LEU | A | 352 | 66.541 | 31.681 | 49.179 | 1.00 | 27.71 |
| ATOM | 3294 | CB | LEU | A | 352 | 67.678 | 31.358 | 48.203 | 1.00 | 22.61 |
| ATOM | 3295 | CG | LEU | A | 352 | 69.026 | 32.057 | 48.373 | 1.00 | 14.74 |
| ATOM | 3296 | CD1 | LEU | A | 352 | 69.958 | 31.612 | 47.280 | 1.00 | 13.29 |
| ATOM | 3297 | CD2 | LEU | A | 352 | 68.853 | 33.554 | 48.306 | 1.00 | 14.68 |
| ATOM | 3298 | C | LEU | A | 352 | 65.348 | 30.775 | 48.886 | 1.00 | 29.37 |
| ATOM | 3299 | O | LEU | A | 352 | 65.375 | 29.573 | 49.179 | 1.00 | 30.14 |
| ATOM | 3300 | N | ALA | A | 353 | 64.309 | 31.349 | 48.289 | 1.00 | 31.25 |
| ATOM | 3302 | CA | ALA | A | 353 | 63.110 | 30.592 | 47.929 | 1.00 | 34.26 |
| ATOM | 3303 | CB | ALA | A | 353 | 61.882 | 31.226 | 48.554 | 1.00 | 35.51 |
| ATOM | 3304 | C | ALA | A | 353 | 62.982 | 30.555 | 46.403 | 1.00 | 36.03 |
| ATOM | 3305 | O | ALA | A | 353 | 62.793 | 31.587 | 45.764 | 1.00 | 37.52 |
| ATOM | 3306 | N | SER | A | 354 | 63.110 | 29.362 | 45.832 | 1.00 | 37.41 |
| ATOM | 3308 | CA | SER | A | 354 | 63.054 | 29.162 | 44.388 | 1.00 | 37.85 |
| ATOM | 3309 | CB | SER | A | 354 | 63.338 | 27.687 | 44.070 | 1.00 | 40.08 |
| ATOM | 3310 | OG | SER | A | 354 | 63.380 | 27.434 | 42.673 | 1.00 | 45.78 |
| ATOM | 3312 | C | SER | A | 354 | 61.744 | 29.601 | 43.741 | 1.00 | 37.70 |
| ATOM | 3313 | O | SER | A | 354 | 60.684 | 29.060 | 44.043 | 1.00 | 40.21 |
| ATOM | 3314 | N | LEU | A | 355 | 61.830 | 30.572 | 42.840 | 1.00 | 36.34 |
| ATOM | 3316 | CA | LEU | A | 355 | 60.657 | 31.074 | 42.137 | 1.00 | 35.04 |
| ATOM | 3317 | CB | LEU | A | 355 | 60.839 | 32.553 | 41.782 | 1.00 | 30.99 |
| ATOM | 3318 | CG | LEU | A | 355 | 60.830 | 33.547 | 42.939 | 1.00 | 28.58 |
| ATOM | 3319 | CD1 | LEU | A | 355 | 61.028 | 34.955 | 42.423 | 1.00 | 28.81 |
| ATOM | 3320 | CD2 | LEU | A | 355 | 59.520 | 33.441 | 43.678 | 1.00 | 30.20 |
| ATOM | 3321 | C | LEU | A | 355 | 60.399 | 30.255 | 40.868 | 1.00 | 36.96 |
| ATOM | 3322 | O | LEU | A | 355 | 59.363 | 29.592 | 40.745 | 1.00 | 36.42 |
| ATOM | 3323 | N | PHE | A | 356 | 61.356 | 30.286 | 39.939 | 1.00 | 37.24 |
| ATOM | 3325 | CA | PHE | A | 356 | 61.251 | 29.556 | 38.673 | 1.00 | 36.22 |
| ATOM | 3326 | CB | PHE | A | 356 | 60.315 | 30.291 | 37.699 | 1.00 | 33.82 |
| ATOM | 3327 | CG | PHE | A | 356 | 60.661 | 31.741 | 37.491 | 1.00 | 30.90 |
| ATOM | 3328 | CD1 | PHE | A | 356 | 61.786 | 32.110 | 36.768 | 1.00 | 29.04 |
| ATOM | 3329 | CD2 | PHE | A | 356 | 59.859 | 32.739 | 38.027 | 1.00 | 30.48 |
| ATOM | 3330 | CE1 | PHE | A | 356 | 62.111 | 33.451 | 36.586 | 1.00 | 28.29 |
| ATOM | 3331 | CE2 | PHE | A | 356 | 60.179 | 34.081 | 37.848 | 1.00 | 30.44 |
| ATOM | 3332 | CZ | PHE | A | 356 | 61.308 | 34.435 | 37.128 | 1.00 | 25.36 |
| ATOM | 3333 | C | PHE | A | 356 | 62.611 | 29.356 | 38.011 | 1.00 | 36.47 |
| ATOM | 3334 | O | PHE | A | 356 | 63.546 | 30.112 | 38.271 | 1.00 | 39.86 |
| ATOM | 3335 | N | LEU | A | 357 | 62.707 | 28.345 | 37.151 | 1.00 | 36.72 |
| ATOM | 3337 | CA | LEU | A | 357 | 63.937 | 28.035 | 36.423 | 1.00 | 35.97 |
| ATOM | 3338 | CB | LEU | A | 357 | 64.358 | 26.585 | 36.670 | 1.00 | 34.25 |
| ATOM | 3339 | CG | LEU | A | 357 | 65.593 | 26.104 | 35.904 | 1.00 | 32.69 |
| ATOM | 3340 | CD1 | LEU | A | 357 | 66.828 | 26.854 | 36.354 | 1.00 | 30.30 |
| ATOM | 3341 | CD2 | LEU | A | 357 | 65.784 | 24.620 | 36.120 | 1.00 | 34.02 |
| ATOM | 3342 | C | LEU | A | 357 | 63.721 | 28.252 | 34.925 | 1.00 | 36.64 |
| ATOM | 3343 | O | LEU | A | 357 | 62.805 | 27.678 | 34.333 | 1.00 | 39.03 |
| ATOM | 3344 | N | ILE | A | 358 | 64.585 | 29.054 | 34.318 | 1.00 | 35.88 |
| ATOM | 3346 | CA | ILE | A | 358 | 64.508 | 29.367 | 32.899 | 1.00 | 34.57 |
| ATOM | 3347 | CB | ILE | A | 358 | 64.510 | 30.903 | 32.672 | 1.00 | 33.20 |
| ATOM | 3348 | CG2 | ILE | A | 358 | 64.653 | 31.239 | 31.208 | 1.00 | 31.65 |
| ATOM | 3349 | CG1 | ILE | A | 358 | 63.220 | 31.510 | 33.207 | 1.00 | 33.82 |
| ATOM | 3350 | CD1 | ILE | A | 358 | 61.978 | 30.907 | 32.602 | 1.00 | 34.80 |
| ATOM | 3351 | C | ILE | A | 358 | 65.677 | 28.748 | 32.143 | 1.00 | 36.24 |
| ATOM | 3352 | O | ILE | A | 358 | 66.784 | 28.628 | 32.666 | 1.00 | 36.67 |
| ATOM | 3353 | N | GLY | A | 359 | 65.405 | 28.338 | 30.912 | 1.00 | 38.02 |
| ATOM | 3355 | CA | GLY | A | 359 | 66.419 | 27.753 | 30.061 | 1.00 | 37.56 |
| ATOM | 3356 | C | GLY | A | 359 | 66.597 | 28.704 | 28.902 | 1.00 | 38.10 |
| ATOM | 3357 | O | GLY | A | 359 | 65.679 | 28.932 | 28.113 | 1.00 | 38.54 |
| ATOM | 3358 | N | MET | A | 360 | 67.769 | 29.305 | 28.830 | 1.00 | 37.31 |
| ATOM | 3360 | CA | MET | A | 360 | 68.060 | 30.252 | 27.785 | 1.00 | 36.92 |
| ATOM | 3361 | CB | MET | A | 360 | 68.515 | 31.560 | 28.405 | 1.00 | 38.52 |
| ATOM | 3362 | CG | MET | A | 360 | 68.813 | 32.656 | 27.421 | 1.00 | 42.50 |
| ATOM | 3363 | SD | MET | A | 360 | 69.679 | 33.987 | 28.246 | 1.00 | 49.37 |
| ATOM | 3364 | CE | MET | A | 360 | 68.747 | 34.125 | 29.725 | 1.00 | 46.11 |
| ATOM | 3365 | C | MET | A | 360 | 69.154 | 29.686 | 26.907 | 1.00 | 38.15 |
| ATOM | 3366 | O | MET | A | 360 | 69.986 | 28.899 | 27.347 | 1.00 | 38.64 |
| ATOM | 3367 | N | HIS | A | 361 | 69.114 | 30.054 | 25.641 | 1.00 | 39.81 |
| ATOM | 3369 | CA | HIS | A | 361 | 70.095 | 29.612 | 24.667 | 1.00 | 41.81 |
| ATOM | 3370 | CB | HIS | A | 361 | 69.766 | 28.215 | 24.131 | 1.00 | 45.81 |
| ATOM | 3371 | CG | HIS | A | 361 | 68.310 | 27.988 | 23.878 | 1.00 | 51.49 |
| ATOM | 3372 | CD2 | HIS | A | 361 | 67.290 | 27.716 | 24.728 | 1.00 | 54.71 |
| ATOM | 3373 | ND1 | HIS | A | 361 | 67.753 | 28.041 | 22.618 | 1.00 | 53.69 |
| ATOM | 3375 | CE1 | HIS | A | 361 | 66.455 | 27.813 | 22.702 | 1.00 | 55.98 |
| ATOM | 3376 | NE2 | HIS | A | 361 | 66.148 | 27.614 | 23.972 | 1.00 | 57.77 |
| ATOM | 3378 | C | HIS | A | 361 | 70.032 | 30.642 | 23.569 | 1.00 | 41.72 |
| ATOM | 3379 | O | HIS | A | 361 | 69.003 | 31.295 | 23.387 | 1.00 | 42.60 |
| ATOM | 3380 | N | THR | A | 362 | 71.132 | 30.816 | 22.858 | 1.00 | 40.54 |
| ATOM | 3382 | CA | THR | A | 362 | 71.168 | 31.803 | 21.803 | 1.00 | 38.28 |
| ATOM | 3383 | CB | THR | A | 362 | 71.452 | 33.206 | 22.399 | 1.00 | 38.27 |
| ATOM | 3384 | OG1 | THR | A | 362 | 71.408 | 34.200 | 21.371 | 1.00 | 40.01 |
| ATOM | 3386 | CG2 | THR | A | 362 | 72.799 | 33.241 | 23.082 | 1.00 | 38.36 |
| ATOM | 3387 | C | THR | A | 362 | 72.244 | 31.391 | 20.818 | 1.00 | 37.84 |
| ATOM | 3388 | O | THR | A | 362 | 72.692 | 30.245 | 20.829 | 1.00 | 38.47 |
| ATOM | 3389 | N | THR | A | 363 | 72.620 | 32.315 | 19.948 | 1.00 | 37.83 |
| ATOM | 3391 | CA | THR | A | 363 | 73.640 | 32.091 | 18.940 | 1.00 | 38.05 |
| ATOM | 3392 | CB | THR | A | 363 | 73.007 | 31.617 | 17.603 | 1.00 | 38.92 |
| ATOM | 3393 | OG1 | THR | A | 363 | 73.993 | 31.618 | 16.567 | 1.00 | 41.55 |
| ATOM | 3395 | CG2 | THR | A | 363 | 71.855 | 32.515 | 17.193 | 1.00 | 42.85 |
| ATOM | 3396 | C | THR | A | 363 | 74.333 | 33.436 | 18.773 | 1.00 | 36.99 |
| ATOM | 3397 | O | THR | A | 363 | 73.668 | 34.467 | 18.705 | 1.00 | 38.50 |
| ATOM | 3398 | N | GLY | A | 364 | 75.660 | 33.438 | 18.763 | 1.00 | 36.22 |
| ATOM | 3400 | CA | GLY | A | 364 | 76.377 | 34.692 | 18.626 | 1.00 | 35.40 |
| ATOM | 3401 | C | GLY | A | 364 | 77.639 | 34.632 | 17.794 | 1.00 | 35.46 |
| ATOM | 3402 | O | GLY | A | 364 | 78.075 | 33.561 | 17.395 | 1.00 | 35.68 |
| ATOM | 3403 | N | SER | A | 365 | 78.216 | 35.795 | 17.522 | 1.00 | 37.37 |
| ATOM | 3405 | CA | SER | A | 365 | 79.434 | 35.884 | 16.738 | 1.00 | 39.81 |
| ATOM | 3406 | CB | SER | A | 365 | 79.399 | 37.121 | 15.840 | 1.00 | 41.52 |
| ATOM | 3407 | OG | SER | A | 365 | 78.345 | 37.028 | 14.895 | 1.00 | 50.49 |
| ATOM | 3409 | C | SER | A | 365 | 80.627 | 35.975 | 17.669 | 1.00 | 40.48 |
| ATOM | 3410 | O | SER | A | 365 | 80.584 | 36.669 | 18.682 | 1.00 | 43.48 |
| ATOM | 3411 | N | MET | A | 366 | 81.685 | 35.259 | 17.330 | 1.00 | 40.04 |
| ATOM | 3413 | CA | MET | A | 366 | 82.898 | 35.271 | 18.118 | 1.00 | 40.25 |
| ATOM | 3414 | CB | MET | A | 366 | 83.254 | 33.836 | 18.509 | 1.00 | 41.64 |
| ATOM | 3415 | CG | MET | A | 366 | 84.524 | 33.688 | 19.310 | 1.00 | 44.41 |
| ATOM | 3416 | SD | MET | A | 366 | 84.378 | 34.496 | 20.881 | 1.00 | 48.93 |
| ATOM | 3417 | CE | MET | A | 366 | 83.512 | 33.252 | 21.833 | 1.00 | 49.58 |
| ATOM | 3418 | C | MET | A | 366 | 83.978 | 35.880 | 17.224 | 1.00 | 41.24 |
| ATOM | 3419 | O | MET | A | 366 | 84.400 | 35.262 | 16.249 | 1.00 | 43.55 |
| ATOM | 3420 | N | GLU | A | 367 | 84.347 | 37.129 | 17.482 | 1.00 | 42.13 |
| ATOM | 3422 | CA | GLU | A | 367 | 85.383 | 37.780 | 16.686 | 1.00 | 43.88 |
| ATOM | 3423 | CB | GLU | A | 367 | 85.148 | 39.288 | 16.608 | 1.00 | 47.79 |
| ATOM | 3424 | CG | GLU | A | 367 | 84.094 | 39.715 | 15.588 | 1.00 | 54.26 |
| ATOM | 3425 | CD | GLU | A | 367 | 84.551 | 39.555 | 14.143 | 1.00 | 57.86 |
| ATOM | 3426 | OE1 | GLU | A | 367 | 85.768 | 39.681 | 13.872 | 1.00 | 60.11 |
| ATOM | 3427 | OE2 | GLU | A | 367 | 83.683 | 39.315 | 13.273 | 1.00 | 59.61 |
| ATOM | 3428 | C | GLU | A | 367 | 86.724 | 37.492 | 17.336 | 1.00 | 43.57 |
| ATOM | 3429 | O | GLU | A | 367 | 86.822 | 37.491 | 18.562 | 1.00 | 44.51 |
| ATOM | 3430 | N | VAL | A | 368 | 87.752 | 37.256 | 16.526 | 1.00 | 42.06 |
| ATOM | 3432 | CA | VAL | A | 368 | 89.077 | 36.944 | 17.047 | 1.00 | 42.38 |
| ATOM | 3433 | CB | VAL | A | 368 | 89.412 | 35.454 | 16.810 | 1.00 | 42.31 |
| ATOM | 3434 | CG1 | VAL | A | 368 | 90.866 | 35.167 | 17.146 | 1.00 | 44.40 |
| ATOM | 3435 | CG2 | VAL | A | 368 | 88.509 | 34.581 | 17.660 | 1.00 | 41.44 |
| ATOM | 3436 | C | VAL | A | 368 | 90.204 | 37.822 | 16.491 | 1.00 | 43.82 |
| ATOM | 3437 | O | VAL | A | 368 | 90.306 | 38.039 | 15.279 | 1.00 | 44.42 |
| ATOM | 3438 | N | SER | A | 369 | 91.051 | 38.312 | 17.392 | 1.00 | 44.15 |
| ATOM | 3440 | CA | SER | A | 369 | 92.182 | 39.160 | 17.032 | 1.00 | 46.20 |
| ATOM | 3441 | CB | SER | A | 369 | 91.838 | 40.636 | 17.254 | 1.00 | 48.08 |
| ATOM | 3442 | OG | SER | A | 369 | 90.692 | 41.011 | 16.508 | 1.00 | 55.65 |
| ATOM | 3444 | C | SER | A | 369 | 93.376 | 38.773 | 17.896 | 1.00 | 46.39 |
| ATOM | 3445 | O | SER | A | 369 | 93.269 | 37.897 | 18.757 | 1.00 | 45.59 |
| ATOM | 3446 | N | ALA | A | 370 | 94.503 | 39.447 | 17.692 | 1.00 | 46.96 |
| ATOM | 3448 | CA | ALA | A | 370 | 95.705 | 39.151 | 18.455 | 1.00 | 46.86 |
| ATOM | 3449 | CB | ALA | A | 370 | 96.659 | 38.334 | 17.615 | 1.00 | 48.51 |
| ATOM | 3450 | C | ALA | A | 370 | 96.401 | 40.399 | 18.961 | 1.00 | 47.76 |
| ATOM | 3451 | O | ALA | A | 370 | 96.526 | 41.384 | 18.234 | 1.00 | 49.78 |
| ATOM | 3452 | N | GLU | A | 371 | 96.833 | 40.346 | 20.215 | 1.00 | 49.05 |
| ATOM | 3454 | CA | GLU | A | 371 | 97.546 | 41.429 | 20.889 | 1.00 | 52.58 |
| ATOM | 3455 | CB | GLU | A | 371 | 96.670 | 42.035 | 21.999 | 1.00 | 55.47 |
| ATOM | 3456 | CG | GLU | A | 371 | 97.394 | 42.954 | 23.010 | 1.00 | 61.82 |
| ATOM | 3457 | CD | GLU | A | 371 | 97.662 | 44.365 | 22.494 | 1.00 | 66.05 |
| ATOM | 3458 | OE1 | GLU | A | 371 | 96.728 | 44.994 | 21.945 | 1.00 | 69.54 |
| ATOM | 3459 | OE2 | GLU | A | 371 | 98.801 | 44.859 | 22.662 | 1.00 | 66.75 |
| ATOM | 3460 | C | GLU | A | 371 | 98.753 | 40.718 | 21.490 | 1.00 | 53.47 |
| ATOM | 3461 | O | GLU | A | 371 | 98.617 | 39.613 | 22.015 | 1.00 | 54.95 |
| ATOM | 3462 | N | SER | A | 372 | 99.926 | 41.337 | 21.418 | 1.00 | 54.76 |
| ATOM | 3464 | CA | SER | A | 372 | 101.145 | 40.718 | 21.932 | 1.00 | 54.93 |
| ATOM | 3465 | CB | SER | A | 372 | 101.080 | 40.570 | 23.462 | 1.00 | 56.19 |
| ATOM | 3466 | OG | SER | A | 372 | 100.788 | 41.803 | 24.098 | 1.00 | 57.76 |
| ATOM | 3468 | C | SER | A | 372 | 101.267 | 39.347 | 21.245 | 1.00 | 54.65 |
| ATOM | 3469 | O | SER | A | 372 | 101.364 | 39.275 | 20.017 | 1.00 | 56.14 |
| ATOM | 3470 | N | ASN | A | 373 | 101.207 | 38.271 | 22.023 | 1.00 | 51.39 |
| ATOM | 3472 | CA | ASN | A | 373 | 101.292 | 36.922 | 21.480 | 1.00 | 48.43 |
| ATOM | 3473 | CB | ASN | A | 373 | 102.606 | 36.249 | 21.891 | 1.00 | 49.96 |
| ATOM | 3474 | CG | ASN | A | 373 | 103.830 | 36.981 | 21.379 | 1.00 | 51.10 |
| ATOM | 3475 | OD1 | ASN | A | 373 | 103.780 | 37.674 | 20.361 | 1.00 | 53.30 |
| ATOM | 3476 | ND2 | ASN | A | 373 | 104.946 | 36.818 | 22.078 | 1.00 | 50.08 |
| ATOM | 3479 | C | ASN | A | 373 | 100.126 | 36.168 | 22.087 | 1.00 | 46.52 |
| ATOM | 3480 | O | ASN | A | 373 | 100.267 | 35.027 | 22.529 | 1.00 | 45.55 |
| ATOM | 3481 | N | ARG | A | 374 | 98.983 | 36.838 | 22.156 | 1.00 | 44.90 |
| ATOM | 3483 | CA | ARG | A | 374 | 97.780 | 36.255 | 22.730 | 1.00 | 43.78 |
| ATOM | 3484 | CB | ARG | A | 374 | 97.293 | 37.082 | 23.929 | 1.00 | 45.07 |
| ATOM | 3485 | CG | ARG | A | 374 | 98.263 | 37.183 | 25.090 | 1.00 | 47.75 |
| ATOM | 3486 | CD | ARG | A | 374 | 97.653 | 37.976 | 26.235 | 1.00 | 52.92 |
| ATOM | 3487 | NE | ARG | A | 374 | 97.293 | 39.342 | 25.857 | 1.00 | 58.67 |
| ATOM | 3489 | CZ | ARG | A | 374 | 97.737 | 40.439 | 26.468 | 1.00 | 62.31 |
| ATOM | 3490 | NH1 | ARG | A | 374 | 98.568 | 40.347 | 27.500 | 1.00 | 63.80 |
| ATOM | 3493 | NH2 | ARG | A | 374 | 97.345 | 41.637 | 26.047 | 1.00 | 62.63 |
| ATOM | 3496 | C | ARG | A | 374 | 96.660 | 36.163 | 21.706 | 1.00 | 41.67 |
| ATOM | 3497 | O | ARG | A | 374 | 96.660 | 36.866 | 20.698 | 1.00 | 42.40 |
| ATOM | 3498 | N | LEU | A | 375 | 95.720 | 35.268 | 21.972 | 1.00 | 39.53 |
| ATOM | 3500 | CA | LEU | A | 375 | 94.568 | 35.063 | 21.119 | 1.00 | 38.25 |
| ATOM | 3501 | CB | LEU | A | 375 | 94.366 | 33.568 | 20.886 | 1.00 | 40.19 |
| ATOM | 3502 | CG | LEU | A | 375 | 93.330 | 33.154 | 19.846 | 1.00 | 42.29 |
| ATOM | 3503 | CD1 | LEU | A | 375 | 93.784 | 33.625 | 18.478 | 1.00 | 42.24 |
| ATOM | 3504 | CD2 | LEU | A | 375 | 93.158 | 31.646 | 19.866 | 1.00 | 41.82 |
| ATOM | 3505 | C | LEU | A | 375 | 93.399 | 35.623 | 21.917 | 1.00 | 37.82 |
| ATOM | 3506 | O | LEU | A | 375 | 93.024 | 35.058 | 22.946 | 1.00 | 38.21 |
| ATOM | 3507 | N | VAL | A | 376 | 92.871 | 36.764 | 21.491 | 1.00 | 38.16 |
| ATOM | 3509 | CA | VAL | A | 376 | 91.752 | 37.384 | 22.193 | 1.00 | 38.06 |
| ATOM | 3510 | CB | VAL | A | 376 | 92.057 | 38.847 | 22.613 | 1.00 | 37.73 |
| ATOM | 3511 | CG1 | VAL | A | 376 | 93.386 | 38.928 | 23.343 | 1.00 | 36.49 |
| ATOM | 3512 | CG2 | VAL | A | 376 | 92.054 | 39.762 | 21.409 | 1.00 | 37.89 |
| ATOM | 3513 | C | VAL | A | 376 | 90.517 | 37.376 | 21.313 | 1.00 | 37.85 |
| ATOM | 3514 | O | VAL | A | 376 | 90.615 | 37.251 | 20.091 | 1.00 | 37.84 |
| ATOM | 3515 | N | GLY | A | 377 | 89.358 | 37.542 | 21.935 | 1.00 | 38.79 |
| ATOM | 3517 | CA | GLY | A | 377 | 88.120 | 37.548 | 21.186 | 1.00 | 38.96 |
| ATOM | 3518 | C | GLY | A | 377 | 87.034 | 38.334 | 21.880 | 1.00 | 38.86 |
| ATOM | 3519 | O | GLY | A | 377 | 87.235 | 38.860 | 22.976 | 1.00 | 40.46 |
| ATOM | 3520 | N | GLU | A | 378 | 85.881 | 38.426 | 21.235 | 1.00 | 39.21 |
| ATOM | 3522 | CA | GLU | A | 378 | 84.752 | 39.143 | 21.793 | 1.00 | 39.89 |
| ATOM | 3523 | CB | GLU | A | 378 | 84.762 | 40.601 | 21.335 | 1.00 | 42.06 |
| ATOM | 3524 | CG | GLU | A | 378 | 83.935 | 41.522 | 22.226 | 1.00 | 47.89 |
| ATOM | 3525 | CD | GLU | A | 378 | 83.384 | 42.725 | 21.488 | 1.00 | 51.11 |
| ATOM | 3526 | OE1 | GLU | A | 378 | 84.142 | 43.357 | 20.718 | 1.00 | 53.99 |
| ATOM | 3527 | OE2 | GLU | A | 378 | 82.184 | 43.031 | 21.679 | 1.00 | 52.17 |
| ATOM | 3528 | C | GLU | A | 378 | 83.481 | 38.456 | 21.315 | 1.00 | 40.11 |
| ATOM | 3529 | O | GLU | A | 378 | 83.352 | 38.132 | 20.133 | 1.00 | 40.78 |
| ATOM | 3530 | N | LEU | A | 379 | 82.569 | 38.211 | 22.249 | 1.00 | 40.26 |
| ATOM | 3532 | CA | LEU | A | 379 | 81.297 | 37.558 | 21.973 | 1.00 | 41.54 |
| ATOM | 3533 | CB | LEU | A | 379 | 80.935 | 36.651 | 23.155 | 1.00 | 43.08 |
| ATOM | 3534 | CG | LEU | A | 379 | 80.208 | 35.314 | 22.991 | 1.00 | 45.34 |
| ATOM | 3535 | CD1 | LEU | A | 379 | 79.839 | 34.798 | 24.377 | 1.00 | 44.87 |
| ATOM | 3536 | CD2 | LEU | A | 379 | 78.966 | 35.444 | 22.136 | 1.00 | 44.17 |
| ATOM | 3537 | C | LEU | A | 379 | 80.199 | 38.613 | 21.799 | 1.00 | 42.68 |
| ATOM | 3538 | O | LEU | A | 379 | 80.179 | 39.623 | 22.507 | 1.00 | 43.44 |
| ATOM | 3539 | N | LYS | A | 380 | 79.303 | 38.384 | 20.845 | 1.00 | 44.09 |
| ATOM | 3541 | CA | LYS | A | 380 | 78.171 | 39.276 | 20.588 | 1.00 | 46.18 |
| ATOM | 3542 | CB | LYS | A | 380 | 78.438 | 40.165 | 19.373 | 1.00 | 48.46 |
| ATOM | 3543 | CG | LYS | A | 380 | 79.444 | 41.279 | 19.660 | 1.00 | 54.25 |
| ATOM | 3544 | CD | LYS | A | 380 | 79.847 | 42.041 | 18.408 | 1.00 | 58.33 |
| ATOM | 3545 | CE | LYS | A | 380 | 80.817 | 43.172 | 18.734 | 1.00 | 60.62 |
| ATOM | 3546 | NZ | LYS | A | 380 | 81.332 | 43.851 | 17.509 | 1.00 | 63.04 |
| ATOM | 3550 | C | LYS | A | 380 | 76.968 | 38.367 | 20.369 | 1.00 | 46.00 |
| ATOM | 3551 | O | LYS | A | 380 | 77.042 | 37.418 | 19.591 | 1.00 | 45.80 |
| ATOM | 3552 | N | LEU | A | 381 | 75.877 | 38.630 | 21.082 | 1.00 | 46.45 |
| ATOM | 3554 | CA | LEU | A | 381 | 74.690 | 37.784 | 20.996 | 1.00 | 46.82 |
| ATOM | 3555 | CB | LEU | A | 381 | 74.115 | 37.545 | 22.400 | 1.00 | 45.07 |
| ATOM | 3556 | CG | LEU | A | 381 | 75.001 | 36.992 | 23.521 | 1.00 | 41.15 |
| ATOM | 3557 | CD1 | LEU | A | 381 | 74.206 | 36.974 | 24.817 | 1.00 | 39.28 |
| ATOM | 3558 | CD2 | LEU | A | 381 | 75.500 | 35.604 | 23.174 | 1.00 | 40.04 |
| ATOM | 3559 | C | LEU | A | 381 | 73.569 | 38.272 | 20.084 | 1.00 | 48.41 |
| ATOM | 3560 | O | LEU | A | 381 | 73.505 | 39.448 | 19.719 | 1.00 | 48.91 |
| ATOM | 3561 | N | ASP | A | 382 | 72.696 | 37.335 | 19.725 | 1.00 | 50.06 |
| ATOM | 3563 | CA | ASP | A | 382 | 71.527 | 37.591 | 18.894 | 1.00 | 54.68 |
| ATOM | 3564 | CB | ASP | A | 382 | 71.371 | 36.488 | 17.845 | 1.00 | 57.78 |
| ATOM | 3565 | CG | ASP | A | 382 | 71.718 | 36.953 | 16.453 | 1.00 | 61.73 |
| ATOM | 3566 | OD1 | ASP | A | 382 | 72.846 | 37.455 | 16.258 | 1.00 | 65.65 |
| ATOM | 3567 | OD2 | ASP | A | 382 | 70.862 | 36.809 | 15.552 | 1.00 | 62.89 |
| ATOM | 3568 | C | ASP | A | 382 | 70.325 | 37.565 | 19.836 | 1.00 | 56.30 |
| ATOM | 3569 | O | ASP | A | 382 | 70.465 | 37.827 | 21.030 | 1.00 | 57.37 |
| ATOM | 3570 | N | ARG | A | 383 | 69.147 | 37.244 | 19.304 | 1.00 | 57.17 |
| ATOM | 3572 | CA | ARG | A | 383 | 67.932 | 37.169 | 20.116 | 1.00 | 56.86 |
| ATOM | 3573 | CB | ARG | A | 383 | 66.710 | 36.913 | 19.221 | 1.00 | 61.20 |
| ATOM | 3574 | CG | ARG | A | 383 | 66.368 | 38.054 | 18.262 | 1.00 | 66.61 |
| ATOM | 3575 | CD | ARG | A | 383 | 65.158 | 37.721 | 17.389 | 1.00 | 68.98 |
| ATOM | 3576 | NE | ARG | A | 383 | 64.674 | 38.887 | 16.647 | 1.00 | 73.50 |
| ATOM | 3578 | CZ | ARG | A | 383 | 64.914 | 39.119 | 15.358 | 1.00 | 76.60 |
| ATOM | 3579 | NH1 | ARG | A | 383 | 65.639 | 38.269 | 14.636 | 1.00 | 77.94 |
| ATOM | 3582 | NH2 | ARG | A | 383 | 64.421 | 40.210 | 14.784 | 1.00 | 77.33 |
| ATOM | 3585 | C | ARG | A | 383 | 68.081 | 36.026 | 21.120 | 1.00 | 54.61 |
| ATOM | 3586 | O | ARG | A | 383 | 68.689 | 35.001 | 20.801 | 1.00 | 56.02 |
| ATOM | 3587 | N | LEU | A | 384 | 67.565 | 36.207 | 22.333 | 1.00 | 50.19 |
| ATOM | 3589 | CA | LEU | A | 384 | 67.654 | 35.163 | 23.353 | 1.00 | 47.21 |
| ATOM | 3590 | CB | LEU | A | 384 | 67.886 | 35.765 | 24.740 | 1.00 | 45.34 |
| ATOM | 3591 | CG | LEU | A | 384 | 69.072 | 36.707 | 24.951 | 1.00 | 44.13 |
| ATOM | 3592 | CD1 | LEU | A | 384 | 69.089 | 37.136 | 26.403 | 1.00 | 48.33 |
| ATOM | 3593 | CD2 | LEU | A | 384 | 70.386 | 36.036 | 24.587 | 1.00 | 44.37 |
| ATOM | 3594 | C | LEU | A | 384 | 66.366 | 34.355 | 23.360 | 1.00 | 46.05 |
| ATOM | 3595 | O | LEU | A | 384 | 65.287 | 34.910 | 23.175 | 1.00 | 46.98 |
| ATOM | 3596 | N | LEU | A | 385 | 66.477 | 33.044 | 23.536 | 1.00 | 45.63 |
| ATOM | 3598 | CA | LEU | A | 385 | 65.299 | 32.190 | 23.564 | 1.00 | 44.83 |
| ATOM | 3599 | CB | LEU | A | 385 | 65.417 | 31.034 | 22.577 | 1.00 | 47.07 |
| ATOM | 3600 | CG | LEU | A | 385 | 65.424 | 31.305 | 21.075 | 1.00 | 49.68 |
| ATOM | 3601 | CD1 | LEU | A | 385 | 66.807 | 31.742 | 20.610 | 1.00 | 50.06 |
| ATOM | 3602 | CD2 | LEU | A | 385 | 65.018 | 30.020 | 20.367 | 1.00 | 53.29 |
| ATOM | 3603 | C | LEU | A | 385 | 65.093 | 31.624 | 24.948 | 1.00 | 43.70 |
| ATOM | 3604 | O | LEU | A | 385 | 65.973 | 30.965 | 25.500 | 1.00 | 46.13 |
| ATOM | 3605 | N | LEU | A | 386 | 63.921 | 31.879 | 25.501 | 1.00 | 41.90 |
| ATOM | 3607 | CA | LEU | A | 386 | 63.586 | 31.399 | 26.819 | 1.00 | 40.93 |
| ATOM | 3608 | CB | LEU | A | 386 | 62.804 | 32.467 | 27.576 | 1.00 | 38.95 |
| ATOM | 3609 | CG | LEU | A | 386 | 63.610 | 33.573 | 28.251 | 1.00 | 38.87 |
| ATOM | 3610 | CD1 | LEU | A | 386 | 64.732 | 34.076 | 27.364 | 1.00 | 36.69 |
| ATOM | 3611 | CD2 | LEU | A | 386 | 62.671 | 34.690 | 28.636 | 1.00 | 38.24 |
| ATOM | 3612 | C | LEU | A | 386 | 62.754 | 30.140 | 26.711 | 1.00 | 41.89 |
| ATOM | 3613 | O | LEU | A | 386 | 62.154 | 29.861 | 25.677 | 1.00 | 44.73 |
| ATOM | 3614 | N | GLU | A | 387 | 62.777 | 29.354 | 27.773 | 1.00 | 42.45 |
| ATOM | 3616 | CA | GLU | A | 387 | 62.006 | 28.133 | 27.867 | 1.00 | 43.22 |
| ATOM | 3617 | CB | GLU | A | 387 | 62.773 | 26.932 | 27.306 | 1.00 | 44.48 |
| ATOM | 3618 | CG | GLU | A | 387 | 61.993 | 25.614 | 27.408 | 1.00 | 46.64 |
| ATOM | 3619 | CD | GLU | A | 387 | 62.699 | 24.436 | 26.762 | 1.00 | 48.74 |
| ATOM | 3620 | OE1 | GLU | A | 387 | 62.622 | 24.305 | 25.520 | 1.00 | 53.50 |
| ATOM | 3621 | OE2 | GLU | A | 387 | 63.311 | 23.629 | 27.495 | 1.00 | 48.42 |
| ATOM | 3622 | C | GLU | A | 387 | 61.791 | 27.966 | 29.359 | 1.00 | 45.59 |
| ATOM | 3623 | O | GLU | A | 387 | 62.697 | 28.232 | 30.150 | 1.00 | 47.68 |
| ATOM | 3624 | N | LEU | A | 388 | 60.574 | 27.618 | 29.750 | 1.00 | 47.18 |
| ATOM | 3626 | CA | LEU | A | 388 | 60.260 | 27.422 | 31.156 | 1.00 | 48.51 |
| ATOM | 3627 | CB | LEU | A | 388 | 58.795 | 27.778 | 31.418 | 1.00 | 49.74 |
| ATOM | 3628 | CG | LEU | A | 388 | 58.298 | 27.776 | 32.865 | 1.00 | 49.97 |
| ATOM | 3629 | CG1 | LEU | A | 388 | 58.957 | 28.902 | 33.656 | 1.00 | 49.70 |
| ATOM | 3630 | CD2 | LEU | A | 388 | 56.788 | 27.935 | 32.871 | 1.00 | 51.43 |
| ATOM | 3631 | C | LEU | A | 388 | 60.513 | 25.953 | 31.468 | 1.00 | 49.89 |
| ATOM | 3632 | O | LEU | A | 388 | 60.033 | 25.071 | 30.754 | 1.00 | 49.90 |
| ATOM | 3633 | N | LYS | A | 389 | 61.290 | 25.688 | 32.511 | 1.00 | 53.07 |
| ATOM | 3635 | CA | LYS | A | 389 | 61.599 | 24.312 | 32.883 | 1.00 | 57.61 |
| ATOM | 3636 | CB | LYS | A | 389 | 63.109 | 24.152 | 33.105 | 1.00 | 58.15 |
| ATOM | 3637 | CG | LYS | A | 389 | 63.933 | 24.486 | 31.864 | 1.00 | 58.89 |
| ATOM | 3638 | CD | LYS | A | 389 | 65.377 | 24.022 | 31.971 | 1.00 | 61.45 |
| ATOM | 3639 | CE | LYS | A | 389 | 66.104 | 24.202 | 30.640 | 1.00 | 63.53 |
| ATOM | 3640 | NZ | LYS | A | 389 | 67.507 | 23.692 | 30.664 | 1.00 | 66.07 |
| ATOM | 3644 | C | LYS | A | 389 | 60.800 | 23.813 | 34.094 | 1.00 | 60.00 |
| ATOM | 3645 | O | LYS | A | 389 | 60.351 | 22.663 | 34.122 | 1.00 | 61.33 |
| ATOM | 3646 | N | HIS | A | 390 | 60.622 | 24.679 | 35.086 | 1.00 | 61.51 |
| ATOM | 3648 | CA | HIS | A | 390 | 59.871 | 24.347 | 36.297 | 1.00 | 63.22 |
| ATOM | 3649 | CB | HIS | A | 390 | 60.713 | 23.474 | 37.242 | 1.00 | 68.01 |
| ATOM | 3650 | CG | HIS | A | 390 | 59.939 | 22.880 | 38.386 | 1.00 | 74.01 |
| ATOM | 3651 | CD2 | HIS | A | 390 | 59.403 | 21.647 | 38.553 | 1.00 | 76.41 |
| ATOM | 3652 | ND1 | HIS | A | 390 | 59.682 | 23.568 | 39.554 | 1.00 | 76.75 |
| ATOM | 3654 | CE1 | HIS | A | 390 | 59.023 | 22.785 | 40.391 | 1.00 | 78.97 |
| ATOM | 3655 | NE2 | HIS | A | 390 | 58.843 | 21.613 | 39.807 | 1.00 | 79.75 |
| ATOM | 3657 | C | HIS | A | 390 | 59.551 | 25.669 | 36.969 | 1.00 | 61.81 |
| ATOM | 3658 | O | HIS | A | 390 | 60.348 | 26.604 | 36.912 | 1.00 | 62.27 |
| ATOM | 3659 | N | SER | A | 391 | 58.375 | 25.762 | 37.572 | 1.00 | 60.92 |
| ATOM | 3661 | CA | SER | A | 391 | 57.978 | 26.980 | 38.257 | 1.00 | 59.76 |
| ATOM | 3662 | CB | SER | A | 391 | 57.005 | 27.793 | 37.403 | 1.00 | 58.20 |
| ATOM | 3663 | OG | SER | A | 391 | 56.639 | 28.996 | 38.059 | 1.00 | 55.50 |
| ATOM | 3665 | C | SER | A | 391 | 57.322 | 26.605 | 39.567 | 1.00 | 59.99 |
| ATOM | 3666 | O | SER | A | 391 | 56.486 | 25.699 | 39.618 | 1.00 | 60.83 |
| ATOM | 3667 | N | ASN | A | 392 | 57.753 | 27.260 | 40.636 | 1.00 | 59.65 |
| ATOM | 3669 | CA | ASN | A | 392 | 57.190 | 27.008 | 41.949 | 1.00 | 59.99 |
| ATOM | 3670 | CB | ASN | A | 392 | 58.241 | 27.234 | 43.039 | 1.00 | 58.88 |
| ATOM | 3671 | CG | ASN | A | 392 | 59.314 | 26.152 | 43.054 | 1.00 | 58.15 |
| ATOM | 3672 | OD1 | ASN | A | 392 | 59.424 | 25.344 | 42.126 | 1.00 | 59.13 |
| ATOM | 3673 | ND2 | ASN | A | 392 | 60.117 | 26.137 | 44.108 | 1.00 | 57.91 |
| ATOM | 3676 | C | ASN | A | 392 | 55.976 | 27.914 | 42.145 | 1.00 | 60.50 |
| ATOM | 3677 | O | ASN | A | 392 | 55.149 | 27.678 | 43.023 | 1.00 | 61.62 |
| ATOM | 3678 | N | ILE | A | 393 | 55.851 | 28.926 | 41.288 | 1.00 | 61.07 |
| ATOM | 3680 | CA | ILE | A | 393 | 54.730 | 29.861 | 41.351 | 1.00 | 60.89 |
| ATOM | 3681 | CB | ILE | A | 393 | 55.212 | 31.333 | 41.306 | 1.00 | 58.61 |
| ATOM | 3682 | CG2 | ILE | A | 393 | 56.083 | 31.634 | 42.510 | 1.00 | 58.38 |
| ATOM | 3683 | CG1 | ILE | A | 393 | 55.948 | 31.622 | 39.995 | 1.00 | 57.06 |
| ATOM | 3684 | CD1 | ILE | A | 393 | 56.245 | 33.087 | 39.775 | 1.00 | 56.51 |
| ATOM | 3685 | C | ILE | A | 393 | 53.700 | 29.632 | 40.228 | 1.00 | 63.24 |
| ATOM | 3686 | O | ILE | A | 393 | 53.049 | 30.574 | 39.772 | 1.00 | 64.68 |
| ATOM | 3687 | N | GLY | A | 394 | 53.556 | 28.382 | 39.786 | 1.00 | 64.29 |
| ATOM | 3689 | CA | GLY | A | 394 | 52.603 | 28.061 | 38.731 | 1.00 | 62.76 |
| ATOM | 3690 | C | GLY | A | 394 | 53.046 | 28.426 | 37.323 | 1.00 | 62.37 |
| ATOM | 3691 | O | GLY | A | 394 | 53.985 | 29.202 | 37.147 | 1.00 | 62.54 |
| ATOM | 3692 | N | PRO | A | 395 | 52.412 | 27.848 | 36.291 | 1.00 | 62.40 |
| ATOM | 3693 | CD | PRO | A | 395 | 51.333 | 26.846 | 36.373 | 1.00 | 63.52 |
| ATOM | 3694 | CA | PRO | A | 395 | 52.758 | 28.130 | 34.894 | 1.00 | 60.73 |
| ATOM | 3695 | CB | PRO | A | 395 | 51.937 | 27.095 | 34.125 | 1.00 | 61.59 |
| ATOM | 3696 | CG | PRO | A | 395 | 50.728 | 26.910 | 34.992 | 1.00 | 63.08 |
| ATOM | 3697 | C | PRO | A | 395 | 52.405 | 29.554 | 34.470 | 1.00 | 59.25 |
| ATOM | 3698 | O | PRO | A | 395 | 51.353 | 30.081 | 34.834 | 1.00 | 59.47 |
| ATOM | 3699 | N | PHE | A | 396 | 53.295 | 30.171 | 33.702 | 1.00 | 57.50 |
| ATOM | 3701 | CA | PHE | A | 396 | 53.087 | 31.530 | 33.220 | 1.00 | 55.80 |
| ATOM | 3702 | CB | PHE | A | 396 | 53.684 | 32.555 | 34.200 | 1.00 | 54.39 |
| ATOM | 3703 | CG | PHE | A | 396 | 55.194 | 32.588 | 34.223 | 1.00 | 51.73 |
| ATOM | 3704 | CD1 | PHE | A | 396 | 55.899 | 33.426 | 33.364 | 1.00 | 49.07 |
| ATOM | 3705 | CD2 | PHE | A | 396 | 55.909 | 31.796 | 35.115 | 1.00 | 50.73 |
| ATOM | 3706 | CE1 | PHE | A | 396 | 57.283 | 33.474 | 33.394 | 1.00 | 47.56 |
| ATOM | 3707 | CE2 | PHE | A | 396 | 57.295 | 31.841 | 35.150 | 1.00 | 47.47 |
| ATOM | 3708 | CZ | PHE | A | 396 | 57.981 | 32.678 | 34.289 | 1.00 | 47.02 |
| ATOM | 3709 | C | PHE | A | 396 | 53.720 | 31.674 | 31.839 | 1.00 | 55.76 |
| ATOM | 3710 | O | PHE | A | 396 | 54.463 | 30.792 | 31.392 | 1.00 | 56.39 |
| ATOM | 3711 | N | PRO | A | 397 | 53.394 | 32.763 | 31.120 | 1.00 | 54.61 |
| ATOM | 3712 | CD | PRO | A | 397 | 52.354 | 33.768 | 31.393 | 1.00 | 54.50 |
| ATOM | 3713 | CA | PRO | A | 397 | 53.966 | 32.967 | 29.787 | 1.00 | 53.13 |
| ATOM | 3714 | CB | PRO | A | 397 | 53.167 | 34.159 | 29.252 | 1.00 | 53.76 |
| ATOM | 3715 | CG | PRO | A | 397 | 51.877 | 34.095 | 30.011 | 1.00 | 53.30 |
| ATOM | 3716 | C | PRO | A | 397 | 55.448 | 33.301 | 29.899 | 1.00 | 51.99 |
| ATOM | 3717 | O | PRO | A | 397 | 55.810 | 34.434 | 30.219 | 1.00 | 51.04 |
| ATOM | 3718 | N | VAL | A | 398 | 56.300 | 32.322 | 29.609 | 1.00 | 50.99 |
| ATOM | 3720 | CA | VAL | A | 398 | 57.747 | 32.506 | 29.686 | 1.00 | 50.88 |
| ATOM | 3721 | CB | VAL | A | 398 | 58.500 | 31.222 | 29.253 | 1.00 | 50.94 |
| ATOM | 3722 | CG1 | VAL | A | 398 | 58.260 | 30.926 | 27.785 | 1.00 | 50.60 |
| ATOM | 3723 | CG2 | VAL | A | 398 | 59.987 | 31.340 | 29.561 | 1.00 | 49.69 |
| ATOM | 3724 | C | VAL | A | 398 | 58.248 | 33.709 | 28.888 | 1.00 | 51.53 |
| ATOM | 3725 | O | VAL | A | 398 | 59.383 | 34.144 | 29.053 | 1.00 | 54.45 |
| ATOM | 3726 | N | GLU | A | 399 | 57.385 | 34.268 | 28.050 | 1.00 | 52.68 |
| ATOM | 3728 | CA | GLU | A | 399 | 57.741 | 35.427 | 27.236 | 1.00 | 52.46 |
| ATOM | 3729 | CB | GLU | A | 399 | 56.807 | 35.533 | 26.020 | 1.00 | 53.50 |
| ATOM | 3730 | CG | GLU | A | 399 | 56.637 | 34.234 | 25.210 | 1.00 | 53.56 |
| ATOM | 3731 | CD | GLU | A | 399 | 55.420 | 33.405 | 25.629 | 1.00 | 53.53 |
| ATOM | 3732 | OE1 | GLU | A | 399 | 54.313 | 33.979 | 25.741 | 1.00 | 54.16 |
| ATOM | 3733 | OE2 | GLU | A | 399 | 55.562 | 32.176 | 25.821 | 1.00 | 52.27 |
| ATOM | 3734 | C | GLU | A | 399 | 57.737 | 36.750 | 28.025 | 1.00 | 51.41 |
| ATOM | 3735 | O | GLU | A | 399 | 58.233 | 37.762 | 27.539 | 1.00 | 52.41 |
| ATOM | 3736 | N | LEU | A | 400 | 57.186 | 36.747 | 29.235 | 1.00 | 49.79 |
| ATOM | 3738 | CA | LEU | A | 400 | 57.148 | 37.955 | 30.060 | 1.00 | 49.44 |
| ATOM | 3739 | CB | LEU | A | 400 | 56.186 | 37.769 | 31.238 | 1.00 | 50.82 |
| ATOM | 3740 | CG | LEU | A | 400 | 54.683 | 38.030 | 31.086 | 1.00 | 49.58 |
| ATOM | 3741 | CD1 | LEU | A | 400 | 54.427 | 39.510 | 30.867 | 1.00 | 50.78 |
| ATOM | 3742 | CD2 | LEU | A | 400 | 54.114 | 37.220 | 29.954 | 1.00 | 50.81 |
| ATOM | 3743 | C | LEU | A | 400 | 58.539 | 38.330 | 30.589 | 1.00 | 50.26 |
| ATOM | 3744 | O | LEU | A | 400 | 58.800 | 39.487 | 30.931 | 1.00 | 50.71 |
| ATOM | 3745 | N | LEU | A | 401 | 59.428 | 37.344 | 30.656 | 1.00 | 50.41 |
| ATOM | 3747 | CA | LEU | A | 401 | 60.790 | 37.548 | 31.143 | 1.00 | 49.44 |
| ATOM | 3748 | CB | LEU | A | 401 | 61.310 | 36.255 | 31.766 | 1.00 | 48.32 |
| ATOM | 3749 | CG | LEU | A | 401 | 60.598 | 35.840 | 33.049 | 1.00 | 46.40 |
| ATOM | 3750 | CD1 | LEU | A | 401 | 60.738 | 34.361 | 33.262 | 1.00 | 45.30 |
| ATOM | 3751 | CD2 | LEU | A | 401 | 61.168 | 36.620 | 34.218 | 1.00 | 48.02 |
| ATOM | 3752 | C | LEU | A | 401 | 61.762 | 38.013 | 30.066 | 1.00 | 49.89 |
| ATOM | 3753 | O | LEU | A | 401 | 62.919 | 38.303 | 30.360 | 1.00 | 51.08 |
| ATOM | 3754 | N | GLN | A | 402 | 61.288 | 38.091 | 28.826 | 1.00 | 51.14 |
| ATOM | 3756 | CA | GLN | A | 402 | 62.107 | 38.511 | 27.686 | 1.00 | 52.43 |
| ATOM | 3757 | CB | GLN | A | 402 | 61.255 | 38.569 | 26.413 | 1.00 | 53.25 |
| ATOM | 3758 | CG | GLN | A | 402 | 60.908 | 37.211 | 25.810 | 1.00 | 55.42 |
| ATOM | 3759 | CD | GLN | A | 402 | 61.966 | 36.697 | 24.851 | 1.00 | 55.66 |
| ATOM | 3760 | OE1 | GLN | A | 402 | 63.075 | 37.229 | 24.780 | 1.00 | 55.93 |
| ATOM | 3761 | NE2 | GLN | A | 402 | 61.619 | 35.667 | 24.090 | 1.00 | 56.71 |
| ATOM | 3764 | C | GLN | A | 402 | 62.800 | 39.855 | 27.890 | 1.00 | 53.23 |
| ATOM | 3765 | O | GLN | A | 402 | 64.009 | 39.977 | 27.684 | 1.00 | 54.78 |
| ATOM | 3766 | N | ASP | A | 403 | 62.031 | 40.866 | 28.275 | 1.00 | 53.89 |
| ATOM | 3768 | CA | ASP | A | 403 | 62.578 | 42.198 | 28.505 | 1.00 | 54.40 |
| ATOM | 3769 | CB | ASP | A | 403 | 61.441 | 43.183 | 28.797 | 1.00 | 58.20 |
| ATOM | 3770 | CG | ASP | A | 403 | 60.524 | 43.394 | 27.597 | 1.00 | 60.37 |
| ATOM | 3771 | OD1 | ASP | A | 403 | 61.001 | 43.915 | 26.563 | 1.00 | 60.48 |
| ATOM | 3772 | OD2 | ASP | A | 403 | 59.325 | 43.045 | 27.691 | 1.00 | 61.61 |
| ATOM | 3773 | C | ASP | A | 403 | 63.615 | 42.204 | 29.638 | 1.00 | 53.27 |
| ATOM | 3774 | O | ASP | A | 403 | 64.669 | 42.836 | 29.525 | 1.00 | 53.59 |
| ATOM | 3775 | N | ILE | A | 404 | 63.318 | 41.487 | 30.719 | 1.00 | 51.11 |
| ATOM | 3777 | CA | ILE | A | 404 | 64.229 | 41.387 | 31.852 | 1.00 | 48.30 |
| ATOM | 3778 | CB | ILE | A | 404 | 63.545 | 40.668 | 33.055 | 1.00 | 45.31 |
| ATOM | 3779 | CG2 | ILE | A | 404 | 64.352 | 39.468 | 33.530 | 1.00 | 47.52 |
| ATOM | 3780 | CG1 | ILE | A | 404 | 63.337 | 41.654 | 34.203 | 1.00 | 42.01 |
| ATOM | 3781 | CD1 | ILE | A | 404 | 64.620 | 42.234 | 34.752 | 1.00 | 36.04 |
| ATOM | 3782 | C | ILE | A | 404 | 65.497 | 40.656 | 31.400 | 1.00 | 48.48 |
| ATOM | 3783 | O | ILE | A | 404 | 66.611 | 41.108 | 31.667 | 1.00 | 51.31 |
| ATOM | 3784 | N | MET | A | 405 | 65.318 | 39.564 | 30.660 | 1.00 | 47.51 |
| ATOM | 3786 | CA | MET | A | 405 | 66.433 | 38.768 | 30.151 | 1.00 | 47.38 |
| ATOM | 3787 | CB | MET | A | 405 | 65.920 | 37.479 | 29.505 | 1.00 | 47.17 |
| ATOM | 3788 | CG | MET | A | 405 | 66.151 | 36.233 | 30.345 | 1.00 | 48.66 |
| ATOM | 3789 | SD | MET | A | 405 | 65.913 | 36.487 | 32.115 | 1.00 | 49.07 |
| ATOM | 3790 | CE | MET | A | 405 | 67.594 | 36.786 | 32.640 | 1.00 | 50.09 |
| ATOM | 3791 | C | MET | A | 405 | 67.291 | 39.540 | 29.160 | 1.00 | 46.62 |
| ATOM | 3792 | O | MET | A | 405 | 68.500 | 39.339 | 29.092 | 1.00 | 48.06 |
| ATOM | 3793 | N | ASN | A | 406 | 66.666 | 40.441 | 28.413 | 1.00 | 46.76 |
| ATOM | 3795 | CA | ASN | A | 406 | 67.383 | 41.239 | 27.433 | 1.00 | 47.68 |
| ATOM | 3796 | CB | ASN | A | 406 | 66.467 | 41.675 | 26.295 | 1.00 | 51.33 |
| ATOM | 3797 | CG | ASN | A | 406 | 66.534 | 40.732 | 25.115 | 1.00 | 54.24 |
| ATOM | 3798 | OD1 | ASN | A | 406 | 67.448 | 40.813 | 24.290 | 1.00 | 55.58 |
| ATOM | 3799 | ND2 | ASN | A | 406 | 65.580 | 39.814 | 25.039 | 1.00 | 54.80 |
| ATOM | 3802 | C | ASN | A | 406 | 68.134 | 42.430 | 27.999 | 1.00 | 46.60 |
| ATOM | 3803 | O | ASN | A | 406 | 68.803 | 43.148 | 27.263 | 1.00 | 48.59 |
| ATOM | 3804 | N | TYR | A | 407 | 67.993 | 42.674 | 29.293 | 1.00 | 45.78 |
| ATOM | 3806 | CA | TYR | A | 407 | 68.732 | 43.760 | 29.922 | 1.00 | 44.83 |
| ATOM | 3807 | CB | TYR | A | 407 | 67.825 | 44.613 | 30.829 | 1.00 | 46.29 |
| ATOM | 3808 | CG | TYR | A | 407 | 68.558 | 45.733 | 31.553 | 1.00 | 45.38 |
| ATOM | 3809 | CD1 | TYR | A | 407 | 68.891 | 46.916 | 30.897 | 1.00 | 45.96 |
| ATOM | 3810 | CE1 | TYR | A | 407 | 69.621 | 47.915 | 31.540 | 1.00 | 47.49 |
| ATOM | 3811 | CD2 | TYR | A | 407 | 68.966 | 45.582 | 32.880 | 1.00 | 45.49 |
| ATOM | 3812 | CE2 | TYR | A | 407 | 69.694 | 46.573 | 33.532 | 1.00 | 44.88 |
| ATOM | 3813 | CZ | TYR | A | 407 | 70.022 | 47.733 | 32.858 | 1.00 | 47.09 |
| ATOM | 3814 | OH | TYR | A | 407 | 70.771 | 48.701 | 33.493 | 1.00 | 48.41 |
| ATOM | 3816 | C | TYR | A | 407 | 69.837 | 43.080 | 30.730 | 1.00 | 43.05 |
| ATOM | 3817 | O | TYR | A | 407 | 71.024 | 43.262 | 30.462 | 1.00 | 42.68 |
| ATOM | 3818 | N | ILE | A | 408 | 69.423 | 42.229 | 31.661 | 1.00 | 41.43 |
| ATOM | 3820 | CA | ILE | A | 408 | 70.332 | 41.490 | 32.523 | 1.00 | 40.32 |
| ATOM | 3821 | CB | ILE | A | 408 | 69.580 | 40.371 | 33.272 | 1.00 | 40.57 |
| ATOM | 3822 | CG2 | ILE | A | 408 | 70.550 | 39.445 | 33.978 | 1.00 | 42.21 |
| ATOM | 3823 | CG1 | ILE | A | 408 | 68.613 | 40.974 | 34.281 | 1.00 | 41.88 |
| ATOM | 3824 | CD1 | ILE | A | 408 | 67.762 | 39.937 | 34.976 | 1.00 | 46.39 |
| ATOM | 3825 | C | ILE | A | 408 | 71.480 | 40.861 | 31.746 | 1.00 | 39.97 |
| ATOM | 3826 | O | ILE | A | 408 | 72.639 | 41.158 | 32.006 | 1.00 | 40.89 |
| ATOM | 3827 | N | VAL | A | 409 | 71.155 | 40.027 | 30.767 | 1.00 | 39.94 |
| ATOM | 3829 | CA | VAL | A | 409 | 72.178 | 39.342 | 29.988 | 1.00 | 39.04 |
| ATOM | 3830 | CB | VAL | A | 409 | 71.560 | 38.354 | 28.961 | 1.00 | 38.16 |
| ATOM | 3831 | CG1 | VAL | A | 409 | 72.656 | 37.687 | 28.142 | 1.00 | 37.45 |
| ATOM | 3832 | CG2 | VAL | A | 409 | 70.741 | 37.291 | 29.686 | 1.00 | 36.14 |
| ATOM | 3833 | C | VAL | A | 409 | 73.252 | 40.227 | 29.343 | 1.00 | 38.82 |
| ATOM | 3834 | O | VAL | A | 409 | 74.426 | 40.093 | 29.675 | 1.00 | 39.64 |
| ATOM | 3835 | N | PRO | A | 410 | 72.881 | 41.145 | 28.432 | 1.00 | 38.89 |
| ATOM | 3836 | CD | PRO | A | 410 | 71.583 | 41.362 | 27.770 | 1.00 | 38.55 |
| ATOM | 3837 | CA | PRO | A | 410 | 73.917 | 41.984 | 27.818 | 1.00 | 38.75 |
| ATOM | 3838 | CB | PRO | A | 410 | 73.207 | 42.562 | 26.597 | 1.00 | 38.19 |
| ATOM | 3839 | CG | PRO | A | 410 | 71.806 | 42.667 | 27.051 | 1.00 | 38.80 |
| ATOM | 3840 | C | PRO | A | 410 | 74.535 | 43.083 | 28.685 | 1.00 | 40.43 |
| ATOM | 3841 | O | PRO | A | 410 | 75.607 | 43.593 | 28.359 | 1.00 | 41.27 |
| ATOM | 3842 | N | ILE | A | 411 | 73.870 | 43.461 | 29.771 | 1.00 | 41.44 |
| ATOM | 3844 | CA | ILE | A | 411 | 74.404 | 44.503 | 30.649 | 1.00 | 41.02 |
| ATOM | 3845 | CB | ILE | A | 411 | 73.285 | 45.380 | 31.239 | 1.00 | 40.99 |
| ATOM | 3846 | CG2 | ILE | A | 411 | 73.890 | 46.497 | 32.086 | 1.00 | 41.83 |
| ATOM | 3847 | CG1 | ILE | A | 411 | 72.418 | 45.967 | 30.122 | 1.00 | 42.47 |
| ATOM | 3848 | CD1 | ILE | A | 411 | 73.112 | 47.005 | 29.264 | 1.00 | 45.28 |
| ATOM | 3849 | C | ILE | A | 411 | 75.212 | 43.923 | 31.809 | 1.00 | 40.15 |
| ATOM | 3850 | O | ILE | A | 411 | 76.254 | 44.460 | 32.186 | 1.00 | 39.92 |
| ATOM | 3851 | N | LEU | A | 412 | 74.716 | 42.831 | 32.379 | 1.00 | 39.13 |
| ATOM | 3853 | CA | LEU | A | 412 | 75.373 | 42.185 | 33.506 | 1.00 | 39.13 |
| ATOM | 3854 | CB | LEU | A | 412 | 74.335 | 41.817 | 34.570 | 1.00 | 39.71 |
| ATOM | 3855 | CG | LEU | A | 412 | 73.493 | 42.941 | 35.179 | 1.00 | 38.70 |
| ATOM | 3856 | CD1 | LEU | A | 412 | 72.548 | 42.362 | 36.220 | 1.00 | 36.24 |
| ATOM | 3857 | CD2 | LEU | A | 412 | 74.403 | 43.986 | 35.802 | 1.00 | 39.15 |
| ATOM | 3858 | C | LEU | A | 412 | 76.209 | 40.945 | 33.175 | 1.00 | 39.67 |
| ATOM | 3859 | O | LEU | A | 412 | 77.322 | 40.796 | 33.677 | 1.00 | 41.17 |
| ATOM | 3860 | N | VAL | A | 413 | 75.678 | 40.060 | 32.336 | 1.00 | 38.13 |
| ATOM | 3862 | CA | VAL | A | 413 | 76.369 | 38.818 | 31.991 | 1.00 | 37.18 |
| ATOM | 3863 | CB | VAL | A | 413 | 75.344 | 37.708 | 31.642 | 1.00 | 38.08 |
| ATOM | 3864 | CG1 | VAL | A | 413 | 76.042 | 36.373 | 31.445 | 1.00 | 39.08 |
| ATOM | 3865 | CG2 | VAL | A | 413 | 74.303 | 37.591 | 32.753 | 1.00 | 35.49 |
| ATOM | 3866 | C | VAL | A | 413 | 77.477 | 38.893 | 30.926 | 1.00 | 36.23 |
| ATOM | 3867 | O | VAL | A | 413 | 78.634 | 38.583 | 31.221 | 1.00 | 36.97 |
| ATOM | 3868 | N | LEU | A | 414 | 77.134 | 39.296 | 29.702 | 1.00 | 34.70 |
| ATOM | 3870 | CA | LEU | A | 414 | 78.108 | 39.394 | 28.610 | 1.00 | 32.37 |
| ATOM | 3871 | CB | LEU | A | 414 | 77.500 | 40.081 | 27.390 | 1.00 | 31.45 |
| ATOM | 3872 | CG | LEU | A | 414 | 77.451 | 39.326 | 26.062 | 1.00 | 31.86 |
| ATOM | 3873 | CG1 | LEU | A | 414 | 77.167 | 40.338 | 24.952 | 1.00 | 34.05 |
| ATOM | 3874 | CD2 | LEU | A | 414 | 78.751 | 38.585 | 25.790 | 1.00 | 30.36 |
| ATOM | 3875 | C | LEU | A | 414 | 79.403 | 40.109 | 28.972 | 1.00 | 33.92 |
| ATOM | 3876 | O | LEU | A | 414 | 80.485 | 39.643 | 28.619 | 1.00 | 36.82 |
| ATOM | 3877 | N | PRO | A | 415 | 79.314 | 41.285 | 29.619 | 1.00 | 34.56 |
| ATOM | 3878 | CD | PRO | A | 415 | 78.103 | 42.090 | 29.858 | 1.00 | 34.83 |
| ATOM | 3879 | CA | PRO | A | 415 | 80.503 | 42.046 | 30.011 | 1.00 | 35.76 |
| ATOM | 3880 | CB | PRO | A | 415 | 79.915 | 43.149 | 30.883 | 1.00 | 35.11 |
| ATOM | 3881 | CG | PRO | A | 415 | 78.673 | 43.467 | 30.151 | 1.00 | 35.76 |
| ATOM | 3882 | C | PRO | A | 415 | 81.583 | 41.263 | 30.748 | 1.00 | 36.22 |
| ATOM | 3883 | O | PRO | A | 415 | 82.760 | 41.387 | 30.418 | 1.00 | 37.32 |
| ATOM | 3884 | N | ARG | A | 416 | 81.194 | 40.466 | 31.739 | 1.00 | 39.00 |
| ATOM | 3886 | CA | ARG | A | 416 | 82.167 | 39.686 | 32.501 | 1.00 | 41.92 |
| ATOM | 3887 | CB | ARG | A | 416 | 81.517 | 39.047 | 33.731 | 1.00 | 46.13 |
| ATOM | 3888 | CG | ARG | A | 416 | 81.111 | 40.038 | 34.823 | 1.00 | 52.86 |
| ATOM | 3889 | CD | ARG | A | 416 | 82.299 | 40.830 | 35.366 | 1.00 | 60.47 |
| ATOM | 3890 | NE | ARG | A | 416 | 83.287 | 39.986 | 36.040 | 1.00 | 69.03 |
| ATOM | 3892 | CZ | ARG | A | 416 | 83.205 | 39.589 | 37.309 | 1.00 | 73.10 |
| ATOM | 3893 | NH1 | ARG | A | 416 | 82.174 | 39.955 | 38.062 | 1.00 | 76.24 |
| ATOM | 3896 | NH2 | ARG | A | 416 | 84.163 | 38.833 | 37.834 | 1.00 | 73.45 |
| ATOM | 3899 | C | ARG | A | 416 | 82.826 | 38.622 | 31.636 | 1.00 | 42.34 |
| ATOM | 3900 | O | ARG | A | 416 | 84.031 | 38.405 | 31.717 | 1.00 | 43.31 |
| ATOM | 3901 | N | VAL | A | 417 | 82.040 | 37.990 | 30.774 | 1.00 | 42.15 |
| ATOM | 3903 | CA | VAL | A | 417 | 82.554 | 36.955 | 29.886 | 1.00 | 41.06 |
| ATOM | 3904 | CB | VAL | A | 417 | 81.418 | 36.302 | 29.091 | 1.00 | 43.59 |
| ATOM | 3905 | CG1 | VAL | A | 417 | 81.976 | 35.261 | 28.127 | 1.00 | 48.62 |
| ATOM | 3906 | CG2 | VAL | A | 417 | 80.414 | 35.672 | 30.038 | 1.00 | 45.02 |
| ATOM | 3907 | C | VAL | A | 417 | 83.581 | 37.500 | 28.902 | 1.00 | 38.49 |
| ATOM | 3908 | O | VAL | A | 417 | 84.658 | 36.929 | 28.729 | 1.00 | 38.46 |
| ATOM | 3909 | N | ASN | A | 418 | 83.236 | 38.598 | 28.243 | 1.00 | 36.89 |
| ATOM | 3911 | CA | ASN | A | 418 | 84.130 | 39.206 | 27.273 | 1.00 | 34.67 |
| ATOM | 3912 | CB | ASN | A | 418 | 83.448 | 40.370 | 26.564 | 1.00 | 36.25 |
| ATOM | 3913 | CG | ASN | A | 418 | 82.506 | 39.913 | 25.475 | 1.00 | 37.58 |
| ATOM | 3914 | OD1 | ASN | A | 418 | 82.585 | 38.776 | 25.014 | 1.00 | 39.99 |
| ATOM | 3915 | ND2 | ASN | A | 418 | 81.613 | 40.796 | 25.055 | 1.00 | 36.92 |
| ATOM | 3918 | C | ASN | A | 418 | 85.426 | 39.671 | 27.896 | 1.00 | 34.68 |
| ATOM | 3919 | O | ASN | A | 418 | 86.421 | 39.831 | 27.199 | 1.00 | 35.38 |
| ATOM | 3920 | N | GLU | A | 419 | 85.426 | 39.895 | 29.206 | 1.00 | 35.06 |
| ATOM | 3922 | CA | GLU | A | 419 | 86.640 | 40.336 | 29.879 | 1.00 | 37.31 |
| ATOM | 3923 | CB | GLU | A | 419 | 86.351 | 40.788 | 31.304 | 1.00 | 41.20 |
| ATOM | 3924 | CG | GLU | A | 419 | 87.557 | 41.433 | 31.972 | 1.00 | 49.99 |
| ATOM | 3925 | CD | GLU | A | 419 | 87.312 | 41.800 | 33.419 | 1.00 | 54.63 |
| ATOM | 3926 | OE1 | GLU | A | 419 | 86.182 | 42.226 | 33.751 | 1.00 | 59.06 |
| ATOM | 3927 | OE2 | GLU | A | 419 | 88.257 | 41.666 | 34.227 | 1.00 | 59.31 |
| ATOM | 3928 | C | GLU | A | 419 | 87.682 | 39.222 | 29.889 | 1.00 | 37.58 |
| ATOM | 3929 | O | GLU | A | 419 | 88.882 | 39.486 | 29.785 | 1.00 | 38.06 |
| ATOM | 3930 | N | LYS | A | 420 | 87.232 | 37.980 | 30.049 | 1.00 | 37.28 |
| ATOM | 3932 | CA | LYS | A | 420 | 88.154 | 36.853 | 30.033 | 1.00 | 37.63 |
| ATOM | 3933 | CB | LYS | A | 420 | 87.492 | 35.591 | 30.587 | 1.00 | 37.80 |
| ATOM | 3934 | CG | LYS | A | 420 | 88.379 | 34.342 | 30.510 | 1.00 | 43.22 |
| ATOM | 3935 | CD | LYS | A | 420 | 89.698 | 34.521 | 31.265 | 1.00 | 46.31 |
| ATOM | 3936 | CE | LYS | A | 420 | 90.650 | 33.352 | 31.031 | 1.00 | 48.33 |
| ATOM | 3937 | NZ | LYS | A | 420 | 91.993 | 33.578 | 31.644 | 1.00 | 48.07 |
| ATOM | 3941 | C | LYS | A | 420 | 88.610 | 36.631 | 28.593 | 1.00 | 38.72 |
| ATOM | 3942 | O | LYS | A | 420 | 89.802 | 36.479 | 28.329 | 1.00 | 41.62 |
| ATOM | 3943 | N | LEU | A | 421 | 87.663 | 36.659 | 27.657 | 1.00 | 38.62 |
| ATOM | 3945 | CA | LEU | A | 421 | 87.975 | 36.466 | 26.246 | 1.00 | 38.52 |
| ATOM | 3946 | CB | LEU | A | 421 | 86.705 | 36.474 | 25.395 | 1.00 | 36.49 |
| ATOM | 3947 | CG | LEU | A | 421 | 85.770 | 35.278 | 25.539 | 1.00 | 34.05 |
| ATOM | 3948 | CD1 | LEU | A | 421 | 84.533 | 35.489 | 24.703 | 1.00 | 31.56 |
| ATOM | 3949 | CD2 | LEU | A | 421 | 86.492 | 34.014 | 25.113 | 1.00 | 36.95 |
| ATOM | 3950 | C | LEU | A | 421 | 88.941 | 37.525 | 25.744 | 1.00 | 40.94 |
| ATOM | 3951 | O | LEU | A | 421 | 89.797 | 37.237 | 24.917 | 1.00 | 43.18 |
| ATOM | 3952 | N | GLN | A | 422 | 88.783 | 38.756 | 26.218 | 1.00 | 44.55 |
| ATOM | 3954 | CA | GLN | A | 422 | 89.672 | 39.844 | 25.820 | 1.00 | 48.56 |
| ATOM | 3955 | CB | GLN | A | 422 | 89.036 | 41.210 | 26.101 | 1.00 | 52.32 |
| ATOM | 3956 | CG | GLN | A | 422 | 88.278 | 41.821 | 24.914 | 1.00 | 58.07 |
| ATOM | 3957 | CD | GLN | A | 422 | 89.197 | 42.459 | 23.863 | 1.00 | 62.49 |
| ATOM | 3958 | OE1 | GLN | A | 422 | 90.361 | 42.770 | 24.133 | 1.00 | 63.36 |
| ATOM | 3959 | NE2 | GLN | A | 422 | 88.661 | 42.673 | 22.665 | 1.00 | 63.04 |
| ATOM | 3962 | C | GLN | A | 422 | 91.037 | 39.725 | 26.501 | 1.00 | 49.05 |
| ATOM | 3963 | O | GLN | A | 422 | 92.013 | 40.323 | 26.045 | 1.00 | 50.57 |
| ATOM | 3964 | N | LYS | A | 423 | 91.096 | 38.996 | 27.615 | 1.00 | 49.71 |
| ATOM | 3966 | CA | LYS | A | 423 | 92.366 | 38.777 | 28.305 | 1.00 | 50.46 |
| ATOM | 3967 | CB | LYS | A | 423 | 92.151 | 38.125 | 29.670 | 1.00 | 52.85 |
| ATOM | 3968 | CG | LYS | A | 423 | 93.442 | 37.862 | 30.434 | 1.00 | 56.39 |
| ATOM | 3969 | CD | LYS | A | 423 | 93.156 | 37.388 | 31.845 | 1.00 | 59.26 |
| ATOM | 3970 | CE | LYS | A | 423 | 92.354 | 38.432 | 32.611 | 1.00 | 60.80 |
| ATOM | 3971 | NZ | LYS | A | 423 | 92.070 | 37.998 | 34.006 | 1.00 | 64.66 |
| ATOM | 3975 | C | LYS | A | 423 | 93.108 | 37.827 | 27.378 | 1.00 | 49.36 |
| ATOM | 3976 | O | LYS | A | 423 | 94.320 | 37.945 | 27.174 | 1.00 | 51.82 |
| ATOM | 3977 | N | GLY | A | 424 | 92.349 | 36.884 | 26.824 | 1.00 | 46.45 |
| ATOM | 3979 | CA | GLY | A | 424 | 92.883 | 35.939 | 25.868 | 1.00 | 40.64 |
| ATOM | 3980 | C | GLY | A | 424 | 93.484 | 34.658 | 26.377 | 1.00 | 38.23 |
| ATOM | 3981 | O | GLY | A | 424 | 93.364 | 34.303 | 27.552 | 1.00 | 36.91 |
| ATOM | 3982 | N | PHE | A | 425 | 94.111 | 33.950 | 25.445 | 1.00 | 36.95 |
| ATOM | 3984 | CA | PHE | A | 425 | 94.768 | 32.684 | 25.712 | 1.00 | 34.69 |
| ATOM | 3985 | CB | PHE | A | 425 | 93.986 | 31.530 | 25.075 | 1.00 | 38.25 |
| ATOM | 3986 | CG | PHE | A | 425 | 92.570 | 31.402 | 25.571 | 1.00 | 44.42 |
| ATOM | 3987 | CD1 | PHE | A | 425 | 92.294 | 31.343 | 26.937 | 1.00 | 44.31 |
| ATOM | 3988 | CD2 | PHE | A | 425 | 91.508 | 31.345 | 24.671 | 1.00 | 46.34 |
| ATOM | 3989 | CE1 | PHE | A | 425 | 90.986 | 31.235 | 27.399 | 1.00 | 45.62 |
| ATOM | 3990 | CE2 | PHE | A | 425 | 90.192 | 31.236 | 25.125 | 1.00 | 48.05 |
| ATOM | 3991 | CZ | PHE | A | 425 | 89.933 | 31.181 | 26.492 | 1.00 | 46.50 |
| ATOM | 3992 | C | PHE | A | 425 | 96.153 | 32.748 | 25.091 | 1.00 | 31.44 |
| ATOM | 3993 | O | PHE | A | 425 | 96.316 | 33.236 | 23.982 | 1.00 | 31.46 |
| ATOM | 3994 | N | PRO | A | 426 | 97.176 | 32.316 | 25.831 | 1.00 | 29.84 |
| ATOM | 3995 | CD | PRO | A | 426 | 97.076 | 31.978 | 27.259 | 1.00 | 30.24 |
| ATOM | 3996 | CA | PRO | A | 426 | 98.571 | 32.299 | 25.396 | 1.00 | 30.20 |
| ATOM | 3997 | CB | PRO | A | 426 | 99.269 | 31.630 | 26.570 | 1.00 | 28.60 |
| ATOM | 3998 | CG | PRO | A | 426 | 98.494 | 32.133 | 27.725 | 1.00 | 31.57 |
| ATOM | 3999 | C | PRO | A | 426 | 98.777 | 31.489 | 24.123 | 1.00 | 31.10 |
| ATOM | 4000 | O | PRO | A | 426 | 98.258 | 30.377 | 23.998 | 1.00 | 34.08 |
| ATOM | 4001 | N | LEU | A | 427 | 99.570 | 32.033 | 23.203 | 1.00 | 29.97 |
| ATOM | 4003 | CA | LEU | A | 427 | 99.878 | 31.377 | 21.935 | 1.00 | 29.50 |
| ATOM | 4004 | CB | LEU | A | 427 | 99.991 | 32.433 | 20.831 | 1.00 | 28.79 |
| ATOM | 4005 | CG | LEU | A | 427 | 99.106 | 32.365 | 19.576 | 1.00 | 31.67 |
| ATOM | 4006 | CD1 | LEU | A | 427 | 97.713 | 31.822 | 19.854 | 1.00 | 29.49 |
| ATOM | 4007 | CD2 | LEU | A | 427 | 99.020 | 33.753 | 18.977 | 1.00 | 30.38 |
| ATOM | 4008 | C | LEU | A | 427 | 101.189 | 30.602 | 22.095 | 1.00 | 28.95 |
| ATOM | 4009 | O | LEU | A | 427 | 102.012 | 30.939 | 22.943 | 1.00 | 30.17 |
| ATOM | 4010 | N | PRO | A | 428 | 101.396 | 29.548 | 21.293 | 1.00 | 29.54 |
| ATOM | 4011 | CD | PRO | A | 428 | 100.502 | 29.048 | 20.234 | 1.00 | 29.81 |
| ATOM | 4012 | CA | PRO | A | 428 | 102.614 | 28.734 | 21.368 | 1.00 | 31.77 |
| ATOM | 4013 | CB | PRO | A | 428 | 102.218 | 27.492 | 20.584 | 1.00 | 30.20 |
| ATOM | 4014 | CG | PRO | A | 428 | 101.382 | 28.073 | 19.490 | 1.00 | 27.41 |
| ATOM | 4015 | C | PRO | A | 428 | 103.843 | 29.405 | 20.753 | 1.00 | 35.74 |
| ATOM | 4016 | O | PRO | A | 428 | 104.361 | 28.950 | 19.731 | 1.00 | 39.47 |
| ATOM | 4017 | N | THR | A | 429 | 104.323 | 30.472 | 21.379 | 1.00 | 36.82 |
| ATOM | 4019 | CA | THR | A | 429 | 105.486 | 31.189 | 20.866 | 1.00 | 36.58 |
| ATOM | 4020 | CB | THR | A | 429 | 105.200 | 32.694 | 20.735 | 1.00 | 38.98 |
| ATOM | 4021 | OG1 | THR | A | 429 | 104.616 | 33.183 | 21.953 | 1.00 | 37.38 |
| ATOM | 4023 | CG2 | THR | A | 429 | 104.262 | 32.956 | 19.565 | 1.00 | 41.95 |
| ATOM | 4024 | C | THR | A | 429 | 106.686 | 31.024 | 21.769 | 1.00 | 35.72 |
| ATOM | 4025 | O | THR | A | 429 | 106.567 | 31.108 | 22.994 | 1.00 | 35.83 |
| ATOM | 4026 | N | PRO | A | 430 | 107.867 | 30.791 | 21.182 | 1.00 | 34.72 |
| ATOM | 4027 | CD | PRO | A | 430 | 108.180 | 30.643 | 19.753 | 1.00 | 36.21 |
| ATOM | 4028 | CA | PRO | A | 430 | 109.069 | 30.629 | 21.995 | 1.00 | 34.61 |
| ATOM | 4029 | CB | PRO | A | 430 | 110.124 | 30.235 | 20.963 | 1.00 | 33.90 |
| ATOM | 4030 | CG | PRO | A | 430 | 109.658 | 30.907 | 19.726 | 1.00 | 33.59 |
| ATOM | 4031 | C | PRO | A | 430 | 109.388 | 31.946 | 22.691 | 1.00 | 35.01 |
| ATOM | 4032 | O | PRO | A | 430 | 108.648 | 32.924 | 22.553 | 1.00 | 37.38 |
| ATOM | 4033 | N | ALA | A | 431 | 110.457 | 31.973 | 23.471 | 1.00 | 34.16 |
| ATOM | 4035 | CA | ALA | A | 431 | 110.824 | 33.190 | 24.173 | 1.00 | 36.53 |
| ATOM | 4036 | CB | ALA | A | 431 | 111.801 | 32.878 | 25.298 | 1.00 | 36.36 |
| ATOM | 4037 | C | ALA | A | 431 | 111.416 | 34.241 | 23.243 | 1.00 | 38.15 |
| ATOM | 4038 | O | ALA | A | 431 | 111.944 | 33.932 | 22.174 | 1.00 | 39.81 |
| ATOM | 4039 | N | ARG | A | 432 | 111.270 | 35.495 | 23.641 | 1.00 | 40.43 |
| ATOM | 4041 | CA | ARG | A | 432 | 111.819 | 36.620 | 22.902 | 1.00 | 41.66 |
| ATOM | 4042 | CB | ARG | A | 432 | 113.349 | 36.537 | 22.887 | 1.00 | 44.65 |
| ATOM | 4043 | CG | ARG | A | 432 | 113.990 | 36.686 | 24.262 | 1.00 | 51.96 |
| ATOM | 4044 | CD | ARG | A | 432 | 115.516 | 36.654 | 24.185 | 1.00 | 59.79 |
| ATOM | 4045 | NE | ARG | A | 432 | 116.029 | 35.372 | 23.700 | 1.00 | 63.72 |
| ATOM | 4047 | CZ | ARG | A | 432 | 117.302 | 34.993 | 23.777 | 1.00 | 67.96 |
| ATOM | 4048 | NH1 | ARG | A | 432 | 118.212 | 35.795 | 24.320 | 1.00 | 70.88 |
| ATOM | 4051 | NH2 | ARG | A | 432 | 117.668 | 33.804 | 23.314 | 1.00 | 68.07 |
| ATOM | 4054 | C | ARG | A | 432 | 111.293 | 36.898 | 21.499 | 1.00 | 40.87 |
| ATOM | 4055 | O | ARG | A | 432 | 111.963 | 37.567 | 20.721 | 1.00 | 42.99 |
| ATOM | 4056 | N | VAL | A | 433 | 110.103 | 36.414 | 21.166 | 1.00 | 40.50 |
| ATOM | 4058 | CA | VAL | A | 433 | 109.538 | 36.693 | 19.847 | 1.00 | 39.69 |
| ATOM | 4059 | CB | VAL | A | 433 | 109.325 | 35.415 | 18.982 | 1.00 | 39.93 |
| ATOM | 4060 | CG1 | VAL | A | 433 | 110.588 | 34.575 | 18.943 | 1.00 | 39.03 |
| ATOM | 4061 | CG2 | VAL | A | 433 | 108.134 | 34.608 | 19.470 | 1.00 | 39.91 |
| ATOM | 4062 | C | VAL | A | 433 | 108.206 | 37.399 | 20.024 | 1.00 | 41.27 |
| ATOM | 4063 | O | VAL | A | 433 | 107.538 | 37.240 | 21.048 | 1.00 | 43.57 |
| ATOM | 4064 | N | GLN | A | 434 | 107.845 | 38.223 | 19.053 | 1.00 | 41.32 |
| ATOM | 4066 | CA | GLN | A | 434 | 106.583 | 38.939 | 19.104 | 1.00 | 41.91 |
| ATOM | 4067 | CB | GLN | A | 434 | 106.802 | 40.411 | 19.454 | 1.00 | 45.76 |
| ATOM | 4068 | CG | GLN | A | 434 | 105.706 | 41.018 | 20.335 | 1.00 | 52.61 |
| ATOM | 4069 | CD | GLN | A | 434 | 105.850 | 40.687 | 21.829 | 1.00 | 56.74 |
| ATOM | 4070 | OE1 | GLN | A | 434 | 105.372 | 41.436 | 22.681 | 1.00 | 58.13 |
| ATOM | 4071 | NE2 | GLN | A | 434 | 106.512 | 39.579 | 22.149 | 1.00 | 57.45 |
| ATOM | 4074 | C | GLN | A | 434 | 106.005 | 38.804 | 17.716 | 1.00 | 40.30 |
| ATOM | 4075 | O | GLN | A | 434 | 106.745 | 38.821 | 16.732 | 1.00 | 40.86 |
| ATOM | 4076 | N | LEU | A | 435 | 104.695 | 38.614 | 17.642 | 1.00 | 39.20 |
| ATOM | 4078 | CA | LEU | A | 435 | 104.018 | 38.439 | 16.366 | 1.00 | 39.99 |
| ATOM | 4079 | CB | LEU | A | 435 | 102.965 | 37.338 | 16.501 | 1.00 | 38.11 |
| ATOM | 4080 | CG | LEU | A | 435 | 103.347 | 36.088 | 17.309 | 1.00 | 37.55 |
| ATOM | 4081 | CD1 | LEU | A | 435 | 102.118 | 35.238 | 17.538 | 1.00 | 37.75 |
| ATOM | 4082 | CD2 | LEU | A | 435 | 104.426 | 35.281 | 16.613 | 1.00 | 36.46 |
| ATOM | 4083 | C | LEU | A | 435 | 103.358 | 39.739 | 15.916 | 1.00 | 41.18 |
| ATOM | 4084 | O | LEU | A | 435 | 102.660 | 40.387 | 16.695 | 1.00 | 42.90 |
| ATOM | 4085 | N | TYR | A | 436 | 103.590 | 40.125 | 14.665 | 1.00 | 42.57 |
| ATOM | 4087 | CA | TYR | A | 436 | 103.004 | 41.348 | 14.116 | 1.00 | 43.80 |
| ATOM | 4088 | CB | TYR | A | 436 | 104.021 | 42.510 | 14.120 | 1.00 | 43.48 |
| ATOM | 4089 | CG | TYR | A | 436 | 105.232 | 42.328 | 13.224 | 1.00 | 43.66 |
| ATOM | 4090 | CD1 | TYR | A | 436 | 106.259 | 41.452 | 13.573 | 1.00 | 43.96 |
| ATOM | 4091 | CE1 | TYR | A | 436 | 107.364 | 41.271 | 12.743 | 1.00 | 44.63 |
| ATOM | 4092 | CD2 | TYR | A | 436 | 105.345 | 43.028 | 12.020 | 1.00 | 43.04 |
| ATOM | 4093 | CE2 | TYR | A | 436 | 106.448 | 42.852 | 11.181 | 1.00 | 43.53 |
| ATOM | 4094 | CZ | TYR | A | 436 | 107.452 | 41.970 | 11.549 | 1.00 | 44.29 |
| ATOM | 4095 | OH | TYR | A | 436 | 108.528 | 41.757 | 10.711 | 1.00 | 44.66 |
| ATOM | 4097 | C | TYR | A | 436 | 102.457 | 41.090 | 12.712 | 1.00 | 43.77 |
| ATOM | 4098 | O | TYR | A | 436 | 102.729 | 40.046 | 12.122 | 1.00 | 45.49 |
| ATOM | 4099 | N | ASN | A | 437 | 101.687 | 42.042 | 12.189 | 1.00 | 45.09 |
| ATOM | 4101 | CA | ASN | A | 437 | 101.069 | 41.932 | 10.865 | 1.00 | 45.97 |
| ATOM | 4102 | CB | ASN | A | 437 | 102.083 | 42.231 | 9.750 | 1.00 | 51.76 |
| ATOM | 4103 | CG | ASN | A | 437 | 102.161 | 43.720 | 9.416 | 1.00 | 57.75 |
| ATOM | 4104 | OD1 | ASN | A | 437 | 102.832 | 44.492 | 10.105 | 1.00 | 59.59 |
| ATOM | 4105 | ND2 | ASN | A | 437 | 101.450 | 44.131 | 8.370 | 1.00 | 60.25 |
| ATOM | 4108 | C | ASN | A | 437 | 100.385 | 40.581 | 10.665 | 1.00 | 44.47 |
| ATOM | 4109 | O | ASN | A | 437 | 100.743 | 39.797 | 9.786 | 1.00 | 44.02 |
| ATOM | 4110 | N | VAL | A | 438 | 99.360 | 40.344 | 11.475 | 1.00 | 44.00 |
| ATOM | 4112 | CA | VAL | A | 438 | 98.622 | 39.091 | 11.444 | 1.00 | 43.09 |
| ATOM | 4113 | CB | VAL | A | 438 | 98.214 | 38.646 | 12.871 | 1.00 | 43.35 |
| ATOM | 4114 | CG1 | VAL | A | 438 | 99.425 | 38.629 | 13.786 | 1.00 | 43.74 |
| ATOM | 4115 | CG2 | VAL | A | 438 | 97.143 | 39.562 | 13.433 | 1.00 | 43.99 |
| ATOM | 4116 | C | VAL | A | 438 | 97.374 | 39.101 | 10.579 | 1.00 | 41.42 |
| ATOM | 4117 | O | VAL | A | 438 | 96.740 | 40.139 | 10.377 | 1.00 | 39.58 |
| ATOM | 4118 | N | VAL | A | 439 | 97.018 | 37.912 | 10.110 | 1.00 | 41.86 |
| ATOM | 4120 | CA | VAL | A | 439 | 95.839 | 37.689 | 9.291 | 1.00 | 43.18 |
| ATOM | 4121 | CB | VAL | A | 439 | 96.202 | 37.377 | 7.829 | 1.00 | 42.54 |
| ATOM | 4122 | CG1 | VAL | A | 439 | 96.568 | 38.646 | 7.104 | 1.00 | 46.06 |
| ATOM | 4123 | CG2 | VAL | A | 439 | 97.351 | 36.391 | 7.770 | 1.00 | 41.82 |
| ATOM | 4124 | C | VAL | A | 439 | 95.117 | 36.482 | 9.866 | 1.00 | 43.37 |
| ATOM | 4125 | O | VAL | A | 439 | 95.733 | 35.446 | 10.103 | 1.00 | 45.02 |
| ATOM | 4126 | N | LEU | A | 440 | 93.829 | 36.631 | 10.141 | 1.00 | 43.45 |
| ATOM | 4128 | CA | LEU | A | 440 | 93.041 | 35.535 | 10.681 | 1.00 | 42.92 |
| ATOM | 4129 | CB | LEU | A | 440 | 92.355 | 35.956 | 11.978 | 1.00 | 42.49 |
| ATOM | 4130 | CG | LEU | A | 440 | 93.220 | 36.062 | 13.234 | 1.00 | 42.93 |
| ATOM | 4131 | CD1 | LEU | A | 440 | 93.920 | 34.745 | 13.462 | 1.00 | 44.28 |
| ATOM | 4132 | CD2 | LEU | A | 440 | 94.231 | 37.181 | 13.111 | 1.00 | 45.59 |
| ATOM | 4133 | C | LEU | A | 440 | 92.008 | 35.132 | 9.643 | 1.00 | 44.03 |
| ATOM | 4134 | O | LEU | A | 440 | 91.017 | 35.831 | 9.446 | 1.00 | 45.13 |
| ATOM | 4135 | N | GLN | A | 441 | 92.268 | 34.029 | 8.947 | 1.00 | 44.00 |
| ATOM | 4137 | CA | GLN | A | 441 | 91.363 | 33.536 | 7.910 | 1.00 | 43.66 |
| ATOM | 4138 | CB | GLN | A | 441 | 92.148 | 33.058 | 6.678 | 1.00 | 48.64 |
| ATOM | 4139 | CG | GLN | A | 441 | 92.881 | 34.113 | 5.860 | 1.00 | 54.68 |
| ATOM | 4140 | CD | GLN | A | 441 | 93.556 | 33.515 | 4.627 | 1.00 | 57.71 |
| ATOM | 4141 | OE1 | GLN | A | 441 | 93.111 | 32.495 | 4.093 | 1.00 | 59.57 |
| ATOM | 4142 | NE2 | GLN | A | 441 | 94.637 | 34.141 | 4.179 | 1.00 | 60.18 |
| ATOM | 4145 | C | GLN | A | 441 | 90.528 | 32.355 | 8.390 | 1.00 | 41.14 |
| ATOM | 4146 | O | GLN | A | 441 | 91.055 | 31.249 | 8.541 | 1.00 | 41.88 |
| ATOM | 4147 | N | PRO | A | 442 | 89.217 | 32.553 | 8.613 | 1.00 | 38.75 |
| ATOM | 4148 | CD | PRO | A | 442 | 88.444 | 33.805 | 8.553 | 1.00 | 39.00 |
| ATOM | 4149 | CA | PRO | A | 442 | 88.372 | 31.442 | 9.065 | 1.00 | 35.99 |
| ATOM | 4150 | CB | PRO | A | 442 | 87.049 | 32.131 | 9.381 | 1.00 | 36.27 |
| ATOM | 4151 | CG | PRO | A | 442 | 87.038 | 33.295 | 8.450 | 1.00 | 38.58 |
| ATOM | 4152 | C | PRO | A | 442 | 88.234 | 30.403 | 7.952 | 1.00 | 34.89 |
| ATOM | 4153 | O | PRO | A | 442 | 88.109 | 30.751 | 6.780 | 1.00 | 37.34 |
| ATOM | 4154 | N | HIS | A | 443 | 88.332 | 29.130 | 8.317 | 1.00 | 32.88 |
| ATOM | 4156 | CA | HIS | A | 443 | 88.242 | 28.036 | 7.361 | 1.00 | 30.71 |
| ATOM | 4157 | CB | HIS | A | 443 | 89.610 | 27.377 | 7.172 | 1.00 | 31.17 |
| ATOM | 4158 | CG | HIS | A | 443 | 90.508 | 28.103 | 6.225 | 1.00 | 29.53 |
| ATOM | 4159 | CD2 | HIS | A | 443 | 91.098 | 27.702 | 5.076 | 1.00 | 27.37 |
| ATOM | 4160 | ND1 | HIS | A | 443 | 90.868 | 29.421 | 6.400 | 1.00 | 31.12 |
| ATOM | 4162 | CE1 | HIS | A | 443 | 91.635 | 29.804 | 5.397 | 1.00 | 30.58 |
| ATOM | 4163 | NE2 | HIS | A | 443 | 91.790 | 28.779 | 4.580 | 1.00 | 29.90 |
| ATOM | 4165 | C | HIS | A | 443 | 87.263 | 27.003 | 7.863 | 1.00 | 31.31 |
| ATOM | 4166 | O | HIS | A | 443 | 86.698 | 27.157 | 8.936 | 1.00 | 36.03 |
| ATOM | 4167 | N | GLN | A | 444 | 87.058 | 25.948 | 7.086 | 1.00 | 33.00 |
| ATOM | 4169 | CA | GLN | A | 444 | 86.136 | 24.896 | 7.481 | 1.00 | 33.08 |
| ATOM | 4170 | CB | GLN | A | 444 | 85.802 | 24.000 | 6.282 | 1.00 | 35.23 |
| ATOM | 4171 | CG | GLN | A | 444 | 84.829 | 22.860 | 6.570 | 1.00 | 37.52 |
| ATOM | 4172 | CD | GLN | A | 444 | 83.436 | 23.337 | 6.954 | 1.00 | 41.28 |
| ATOM | 4173 | OE1 | GLN | A | 444 | 83.142 | 24.531 | 6.941 | 1.00 | 42.98 |
| ATOM | 4174 | NE2 | GLN | A | 444 | 82.570 | 22.395 | 7.298 | 1.00 | 44.68 |
| ATOM | 4177 | C | GLN | A | 444 | 86.735 | 24.069 | 8.613 | 1.00 | 32.22 |
| ATOM | 4178 | O | GLN | A | 444 | 87.720 | 23.360 | 8.416 | 1.00 | 34.24 |
| ATOM | 4179 | N | ASN | A | 445 | 86.175 | 24.230 | 9.808 | 1.00 | 29.86 |
| ATOM | 4181 | CA | ASN | A | 445 | 86.584 | 23.492 | 11.003 | 1.00 | 28.34 |
| ATOM | 4182 | CB | ASN | A | 445 | 86.628 | 21.984 | 10.714 | 1.00 | 27.65 |
| ATOM | 4183 | CG | ASN | A | 445 | 85.272 | 21.427 | 10.319 | 1.00 | 28.84 |
| ATOM | 4184 | OD1 | ASN | A | 445 | 85.155 | 20.637 | 9.385 | 1.00 | 33.08 |
| ATOM | 4185 | ND2 | ASN | A | 445 | 84.233 | 21.850 | 11.022 | 1.00 | 32.64 |
| ATOM | 4188 | C | ASN | A | 445 | 87.863 | 23.950 | 11.709 | 1.00 | 27.86 |
| ATOM | 4189 | O | ASN | A | 445 | 88.318 | 23.306 | 12.658 | 1.00 | 30.31 |
| ATOM | 4190 | N | PHE | A | 446 | 88.436 | 25.064 | 11.272 | 1.00 | 26.55 |
| ATOM | 4192 | CA | PHE | A | 446 | 89.639 | 25.582 | 11.904 | 1.00 | 25.16 |
| ATOM | 4193 | CB | PHE | A | 446 | 90.858 | 24.688 | 11.626 | 1.00 | 28.26 |
| ATOM | 4194 | CG | PHE | A | 446 | 91.407 | 24.796 | 10.233 | 1.00 | 29.75 |
| ATOM | 4195 | CD1 | PHE | A | 446 | 90.953 | 23.956 | 9.223 | 1.00 | 29.14 |
| ATOM | 4196 | CD2 | PHE | A | 446 | 92.413 | 25.711 | 9.940 | 1.00 | 32.05 |
| ATOM | 4197 | CE1 | PHE | A | 446 | 91.492 | 24.020 | 7.950 | 1.00 | 26.24 |
| ATOM | 4198 | CE2 | PHE | A | 446 | 92.961 | 25.784 | 8.662 | 1.00 | 30.94 |
| ATOM | 4199 | CZ | PHE | A | 446 | 92.500 | 24.936 | 7.667 | 1.00 | 29.55 |
| ATOM | 4200 | C | PHE | A | 446 | 89.906 | 27.020 | 11.506 | 1.00 | 25.07 |
| ATOM | 4201 | O | PHE | A | 446 | 89.372 | 27.508 | 10.513 | 1.00 | 24.16 |
| ATOM | 4202 | N | LEU | A | 447 | 90.718 | 27.697 | 12.306 | 1.00 | 26.36 |
| ATOM | 4204 | CA | LEU | A | 447 | 91.059 | 29.091 | 12.076 | 1.00 | 25.79 |
| ATOM | 4205 | CB | LEU | A | 447 | 90.825 | 29.880 | 13.365 | 1.00 | 27.64 |
| ATOM | 4206 | CG | LEU | A | 447 | 91.097 | 31.384 | 13.422 | 1.00 | 28.75 |
| ATOM | 4207 | CD1 | LEU | A | 447 | 90.193 | 32.125 | 12.449 | 1.00 | 27.40 |
| ATOM | 4208 | CD2 | LEU | A | 447 | 90.863 | 31.875 | 14.843 | 1.00 | 25.78 |
| ATOM | 4209 | C | LEU | A | 447 | 92.508 | 29.221 | 11.635 | 1.00 | 25.68 |
| ATOM | 4210 | O | LEU | A | 447 | 93.398 | 28.619 | 12.223 | 1.00 | 26.90 |
| ATOM | 4211 | N | LEU | A | 448 | 92.738 | 30.000 | 10.589 | 1.00 | 26.87 |
| ATOM | 4213 | CA | LEU | A | 448 | 94.080 | 30.199 | 10.084 | 1.00 | 26.96 |
| ATOM | 4214 | CB | LEU | A | 448 | 94.074 | 30.247 | 8.559 | 1.00 | 26.42 |
| ATOM | 4215 | CG | LEU | A | 448 | 95.432 | 30.045 | 7.886 | 1.00 | 25.05 |
| ATOM | 4216 | CD1 | LEU | A | 448 | 96.040 | 28.738 | 8.331 | 1.00 | 25.88 |
| ATOM | 4217 | CD2 | LEU | A | 448 | 95.273 | 30.049 | 6.396 | 1.00 | 29.10 |
| ATOM | 4218 | C | LEU | A | 448 | 94.627 | 31.493 | 10.651 | 1.00 | 29.88 |
| ATOM | 4219 | O | LEU | A | 448 | 94.005 | 32.552 | 10.530 | 1.00 | 28.70 |
| ATOM | 4220 | N | PHE | A | 449 | 95.788 | 31.386 | 11.283 | 1.00 | 32.23 |
| ATOM | 4222 | CA | PHE | A | 449 | 96.469 | 32.511 | 11.897 | 1.00 | 32.39 |
| ATOM | 4223 | CB | PHE | A | 449 | 96.655 | 32.234 | 13.394 | 1.00 | 31.41 |
| ATOM | 4224 | CG | PHE | A | 449 | 97.511 | 33.243 | 14.100 | 1.00 | 31.44 |
| ATOM | 4225 | CD1 | PHE | A | 449 | 98.897 | 33.160 | 14.046 | 1.00 | 32.18 |
| ATOM | 4226 | CD2 | PHE | A | 449 | 96.934 | 34.296 | 14.797 | 1.00 | 35.10 |
| ATOM | 4227 | CE1 | PHE | A | 449 | 99.695 | 34.111 | 14.671 | 1.00 | 32.81 |
| ATOM | 4228 | CE2 | PHE | A | 449 | 97.725 | 35.257 | 15.429 | 1.00 | 34.26 |
| ATOM | 4229 | CZ | PHE | A | 449 | 99.106 | 35.162 | 15.363 | 1.00 | 33.12 |
| ATOM | 4230 | C | PHE | A | 449 | 97.829 | 32.685 | 11.228 | 1.00 | 34.46 |
| ATOM | 4231 | O | PHE | A | 449 | 98.717 | 31.865 | 11.420 | 1.00 | 38.92 |
| ATOM | 4232 | N | GLY | A | 450 | 97.990 | 33.738 | 10.439 | 1.00 | 34.70 |
| ATOM | 4234 | CA | GLY | A | 450 | 99.262 | 33.982 | 9.781 | 1.00 | 33.42 |
| ATOM | 4235 | C | GLY | A | 450 | 99.901 | 35.207 | 10.397 | 1.00 | 35.08 |
| ATOM | 4236 | O | GLY | A | 450 | 99.194 | 36.140 | 10.784 | 1.00 | 36.42 |
| ATOM | 4237 | N | ALA | A | 451 | 101.225 | 35.234 | 10.491 | 1.00 | 33.89 |
| ATOM | 4239 | CA | ALA | A | 451 | 101.889 | 36.383 | 11.092 | 1.00 | 33.95 |
| ATOM | 4240 | CB | ALA | A | 451 | 101.649 | 36.384 | 12.587 | 1.00 | 31.78 |
| ATOM | 4241 | C | ALA | A | 451 | 103.382 | 36.454 | 10.815 | 1.00 | 34.43 |
| ATOM | 4242 | O | ALA | A | 451 | 103.986 | 35.489 | 10.335 | 1.00 | 36.02 |
| ATOM | 4243 | N | ASP | A | 452 | 103.954 | 37.628 | 11.055 | 1.00 | 33.44 |
| ATOM | 4245 | CA | ASP | A | 452 | 105.381 | 37.847 | 10.885 | 1.00 | 35.01 |
| ATOM | 4246 | CB | ASP | A | 452 | 105.670 | 39.186 | 10.196 | 1.00 | 37.76 |
| ATOM | 4247 | CG | ASP | A | 452 | 105.437 | 39.144 | 8.696 | 1.00 | 40.65 |
| ATOM | 4248 | OD1 | ASP | A | 452 | 105.743 | 38.106 | 8.074 | 1.00 | 42.78 |
| ATOM | 4249 | OD2 | ASP | A | 452 | 104.961 | 40.159 | 8.134 | 1.00 | 42.90 |
| ATOM | 4250 | C | ASP | A | 452 | 105.913 | 37.875 | 12.306 | 1.00 | 35.28 |
| ATOM | 4251 | O | ASP | A | 452 | 105.196 | 38.247 | 13.237 | 1.00 | 33.85 |
| ATOM | 4252 | N | VAL | A | 453 | 107.174 | 37.509 | 12.473 | 1.00 | 37.44 |
| ATOM | 4254 | CA | VAL | A | 453 | 107.771 | 37.468 | 13.798 | 1.00 | 38.73 |
| ATOM | 4255 | CB | VAL | A | 453 | 108.253 | 36.034 | 14.138 | 1.00 | 39.37 |
| ATOM | 4256 | CG1 | VAL | A | 453 | 108.678 | 35.955 | 15.591 | 1.00 | 40.45 |
| ATOM | 4257 | CG2 | VAL | A | 453 | 107.169 | 35.004 | 13.819 | 1.00 | 37.45 |
| ATOM | 4258 | C | VAL | A | 453 | 108.970 | 38.398 | 13.899 | 1.00 | 39.49 |
| ATOM | 4259 | O | VAL | A | 453 | 109.669 | 38.641 | 12.914 | 1.00 | 40.46 |
| ATOM | 4260 | N | VAL | A | 454 | 109.190 | 38.929 | 15.092 | 1.00 | 39.41 |
| ATOM | 4262 | CA | VAL | A | 454 | 110.324 | 39.796 | 15.331 | 1.00 | 40.55 |
| ATOM | 4263 | CB | VAL | A | 454 | 109.895 | 41.280 | 15.511 | 1.00 | 41.60 |
| ATOM | 4264 | CG1 | VAL | A | 454 | 108.859 | 41.423 | 16.605 | 1.00 | 42.90 |
| ATOM | 4265 | CG2 | VAL | A | 454 | 111.105 | 42.149 | 15.798 | 1.00 | 43.42 |
| ATOM | 4266 | C | VAL | A | 454 | 111.037 | 39.256 | 16.561 | 1.00 | 41.20 |
| ATOM | 4267 | O | VAL | A | 454 | 110.440 | 39.115 | 17.626 | 1.00 | 41.61 |
| ATOM | 4268 | N | TYR | A | 455 | 112.284 | 38.851 | 16.376 | 1.00 | 42.91 |
| ATOM | 4270 | CA | TYR | A | 455 | 113.084 | 38.309 | 17.460 | 1.00 | 45.62 |
| ATOM | 4271 | CB | TYR | A | 455 | 114.015 | 37.227 | 16.908 | 1.00 | 44.83 |
| ATOM | 4272 | CG | TYR | A | 455 | 115.038 | 36.684 | 17.881 | 1.00 | 44.58 |
| ATOM | 4273 | CD1 | TYR | A | 455 | 114.682 | 35.774 | 18.875 | 1.00 | 43.02 |
| ATOM | 4274 | CE1 | TYR | A | 455 | 115.644 | 35.246 | 19.739 | 1.00 | 44.27 |
| ATOM | 4275 | CD2 | TYR | A | 455 | 116.377 | 37.053 | 17.778 | 1.00 | 46.42 |
| ATOM | 4276 | CE2 | TYR | A | 455 | 117.341 | 36.535 | 18.631 | 1.00 | 46.69 |
| ATOM | 4277 | CZ | TYR | A | 455 | 116.973 | 35.634 | 19.607 | 1.00 | 46.43 |
| ATOM | 4278 | OH | TYR | A | 455 | 117.944 | 35.141 | 20.445 | 1.00 | 49.56 |
| ATOM | 4280 | C | TYR | A | 455 | 113.864 | 39.443 | 18.128 | 1.00 | 49.88 |
| ATOM | 4281 | O | TYR | A | 455 | 114.558 | 40.213 | 17.455 | 1.00 | 49.95 |
| ATOM | 4282 | N | LYS | A | 456 | 113.694 | 39.552 | 19.444 | 1.00 | 54.41 |
| ATOM | 4284 | CA | LYS | A | 456 | 114.337 | 40.570 | 20.270 | 1.00 | 59.38 |
| ATOM | 4285 | CB | LYS | A | 456 | 113.760 | 40.514 | 21.693 | 1.00 | 61.68 |
| ATOM | 4286 | CG | LYS | A | 456 | 114.401 | 41.475 | 22.692 | 1.00 | 66.56 |
| ATOM | 4287 | CD | LYS | A | 456 | 114.016 | 41.153 | 24.140 | 1.00 | 68.38 |
| ATOM | 4288 | CE | LYS | A | 456 | 112.538 | 41.396 | 24.409 | 1.00 | 70.09 |
| ATOM | 4289 | NZ | LYS | A | 456 | 112.154 | 41.067 | 25.812 | 1.00 | 71.48 |
| ATOM | 4293 | C | LYS | A | 456 | 115.855 | 40.382 | 20.311 | 1.00 | 62.37 |
| ATOM | 4294 | OT1 | LYS | A | 456 | 116.324 | 39.430 | 20.976 | 1.00 | 64.57 |
| ATOM | 4295 | OT2 | LYS | A | 456 | 116.559 | 41.196 | 19.673 | 1.00 | 66.00 |
| ATOM | 4296 | C1 | PC | A | 777 | 121.817 | 32.468 | 19.343 | 1.00 | 72.53 |
| ATOM | 4297 | C2 | PC | A | 777 | 121.094 | 31.121 | 19.465 | 1.00 | 63.82 |
| ATOM | 4298 | C3 | PC | A | 777 | 119.917 | 31.017 | 18.492 | 1.00 | 59.16 |
| ATOM | 4299 | C4 | PC | A | 777 | 123.302 | 33.354 | 23.035 | 1.00 | 94.69 |
| ATOM | 4300 | C5 | PC | A | 777 | 124.151 | 34.553 | 23.445 | 1.00 | 98.10 |
| ATOM | 4301 | C6 | PC | A | 777 | 125.683 | 33.126 | 24.652 | 1.00 | 99.68 |
| ATOM | 4302 | C7 | PC | A | 777 | 126.361 | 35.328 | 24.003 | 1.00 | 100.00 |
| ATOM | 4303 | C8 | PC | A | 777 | 126.086 | 33.627 | 22.335 | 1.00 | 100.00 |
| ATOM | 4304 | C31 | PC | A | 777 | 122.736 | 29.776 | 18.047 | 1.00 | 54.84 |
| ATOM | 4305 | C32 | PC | A | 777 | 122.337 | 30.654 | 16.869 | 1.00 | 51.64 |
| ATOM | 4306 | C33 | PC | A | 777 | 122.254 | 29.878 | 15.549 | 1.00 | 47.76 |
| ATOM | 4307 | C34 | PC | A | 777 | 123.590 | 29.237 | 15.169 | 1.00 | 42.03 |
| ATOM | 4308 | C35 | PC | A | 777 | 123.429 | 28.173 | 14.085 | 1.00 | 39.41 |
| ATOM | 4309 | C36 | PC | A | 777 | 122.582 | 28.674 | 12.916 | 1.00 | 36.18 |
| ATOM | 4310 | C37 | PC | A | 777 | 121.581 | 27.622 | 12.463 | 1.00 | 33.16 |
| ATOM | 4311 | C38 | PC | A | 777 | 120.679 | 28.158 | 11.377 | 1.00 | 31.40 |
| ATOM | 4312 | C39 | PC | A | 777 | 119.610 | 29.062 | 11.951 | 1.00 | 36.47 |
| ATOM | 4313 | C40 | PC | A | 777 | 118.543 | 28.263 | 12.682 | 1.00 | 40.19 |
| ATOM | 4314 | C41 | PC | A | 777 | 117.191 | 28.383 | 12.004 | 1.00 | 39.41 |
| ATOM | 4315 | C42 | PC | A | 777 | 116.468 | 27.050 | 11.956 | 1.00 | 41.86 |
| ATOM | 4316 | C43 | PC | A | 777 | 115.409 | 26.958 | 13.041 | 1.00 | 43.49 |
| ATOM | 4317 | C44 | PC | A | 777 | 114.326 | 25.951 | 12.669 | 1.00 | 46.22 |
| ATOM | 4318 | C45 | PC | A | 777 | 113.126 | 26.048 | 13.604 | 1.00 | 47.77 |
| ATOM | 4319 | C46 | PC | A | 777 | 112.271 | 24.786 | 13.561 | 1.00 | 48.67 |
| ATOM | 4320 | C47 | PC | A | 777 | 111.948 | 24.277 | 14.964 | 1.00 | 48.62 |
| ATOM | 4321 | C48 | PC | A | 777 | 110.492 | 24.449 | 15.368 | 1.00 | 45.72 |
| ATOM | 4322 | C11 | PC | A | 777 | 118.509 | 32.389 | 17.081 | 1.00 | 49.62 |
| ATOM | 4323 | C12 | PC | A | 777 | 117.647 | 31.121 | 17.072 | 1.00 | 45.91 |
| ATOM | 4324 | C13 | PC | A | 777 | 116.542 | 31.091 | 16.014 | 1.00 | 42.05 |
| ATOM | 4325 | C14 | PC | A | 777 | 115.753 | 32.392 | 15.973 | 1.00 | 37.88 |
| ATOM | 4326 | C15 | PC | A | 777 | 114.269 | 32.149 | 16.004 | 1.00 | 39.34 |
| ATOM | 4327 | C16 | PC | A | 777 | 113.573 | 32.877 | 14.874 | 1.00 | 39.38 |
| ATOM | 4328 | C17 | PC | A | 777 | 112.066 | 32.688 | 14.951 | 1.00 | 43.09 |
| ATOM | 4329 | C18 | PC | A | 777 | 111.446 | 32.535 | 13.566 | 1.00 | 45.42 |
| ATOM | 4330 | C19 | PC | A | 777 | 111.500 | 31.093 | 13.082 | 1.00 | 47.16 |
| ATOM | 4331 | C20 | PC | A | 777 | 110.158 | 30.413 | 13.236 | 1.00 | 46.62 |
| ATOM | 4332 | C21 | PC | A | 777 | 109.917 | 30.004 | 14.673 | 1.00 | 46.65 |
| ATOM | 4333 | C22 | PC | A | 777 | 108.444 | 29.836 | 14.942 | 1.00 | 46.97 |
| ATOM | 4334 | C23 | PC | A | 777 | 107.922 | 30.916 | 15.868 | 1.00 | 47.30 |
| ATOM | 4335 | C24 | PC | A | 777 | 106.409 | 30.835 | 16.001 | 1.00 | 50.05 |
| ATOM | 4336 | C25 | PC | A | 777 | 105.996 | 29.898 | 17.121 | 1.00 | 50.59 |
| ATOM | 4337 | C26 | PC | A | 777 | 105.783 | 28.490 | 16.612 | 1.00 | 53.56 |
| ATOM | 4338 | C27 | PC | A | 777 | 106.972 | 27.593 | 16.941 | 1.00 | 56.03 |
| ATOM | 4339 | C28 | PC | A | 777 | 107.348 | 26.625 | 15.831 | 1.00 | 55.75 |
| ATOM | 4340 | O11 | PC | A | 777 | 118.194 | 33.427 | 16.467 | 1.00 | 52.98 |
| ATOM | 4341 | O31 | PC | A | 777 | 123.576 | 28.862 | 17.906 | 1.00 | 56.48 |
| ATOM | 4342 | O2 | PC | A | 777 | 122.068 | 30.020 | 19.350 | 1.00 | 61.36 |
| ATOM | 4343 | O3 | PC | A | 777 | 119.731 | 32.307 | 17.866 | 1.00 | 53.04 |
| ATOM | 4344 | O1P | PC | A | 777 | 121.661 | 35.196 | 20.857 | 1.00 | 89.42 |
| ATOM | 4345 | O2P | PC | A | 777 | 120.059 | 33.339 | 21.280 | 1.00 | 90.46 |
| ATOM | 4346 | O3P | PC | A | 777 | 122.419 | 32.815 | 20.622 | 1.00 | 84.15 |
| ATOM | 4347 | O4P | PC | A | 777 | 121.916 | 33.769 | 22.889 | 1.00 | 91.71 |
| ATOM | 4348 | N | PC | A | 777 | 125.571 | 34.153 | 23.607 | 1.00 | 99.82 |
| ATOM | 4349 | P | PC | A | 777 | 121.516 | 33.779 | 21.409 | 1.00 | 89.00 |
| ATOM | 4350 | C1 | PC | A | 778 | 87.797 | 32.791 | 33.686 | 1.00 | 88.29 |
| ATOM | 4351 | C2 | PC | A | 778 | 86.274 | 32.868 | 33.502 | 1.00 | 83.64 |
| ATOM | 4352 | C3 | PC | A | 778 | 85.739 | 31.590 | 32.830 | 1.00 | 80.89 |
| ATOM | 4353 | C31 | PC | A | 778 | 84.282 | 33.619 | 34.787 | 1.00 | 76.37 |
| ATOM | 4354 | C32 | PC | A | 778 | 83.704 | 33.859 | 33.391 | 1.00 | 70.52 |
| ATOM | 4355 | C33 | PC | A | 778 | 82.344 | 33.215 | 33.178 | 1.00 | 63.44 |
| ATOM | 4356 | C34 | PC | A | 778 | 81.311 | 34.250 | 32.803 | 1.00 | 56.98 |
| ATOM | 4357 | C35 | PC | A | 778 | 80.231 | 34.364 | 33.854 | 1.00 | 51.89 |
| ATOM | 4358 | C36 | PC | A | 778 | 79.088 | 33.400 | 33.593 | 1.00 | 48.39 |
| ATOM | 4359 | C37 | PC | A | 778 | 78.611 | 33.453 | 32.155 | 1.00 | 47.51 |
| ATOM | 4360 | C38 | PC | A | 778 | 77.593 | 32.352 | 31.868 | 1.00 | 47.46 |
| ATOM | 4361 | C39 | PC | A | 778 | 78.231 | 31.153 | 31.171 | 1.00 | 45.28 |
| ATOM | 4362 | C40 | PC | A | 778 | 78.154 | 31.261 | 29.654 | 1.00 | 43.25 |
| ATOM | 4363 | C41 | PC | A | 778 | 79.381 | 31.957 | 29.099 | 1.00 | 40.88 |
| ATOM | 4364 | C42 | PC | A | 778 | 79.324 | 32.082 | 27.589 | 1.00 | 42.02 |
| ATOM | 4365 | C43 | PC | A | 778 | 78.064 | 32.803 | 27.130 | 1.00 | 43.52 |
| ATOM | 4366 | C44 | PC | A | 778 | 77.971 | 34.223 | 27.684 | 1.00 | 42.21 |
| ATOM | 4367 | C45 | PC | A | 778 | 76.563 | 34.546 | 28.171 | 1.00 | 42.30 |
| ATOM | 4368 | C46 | PC | A | 778 | 75.523 | 34.335 | 27.077 | 1.00 | 42.97 |
| ATOM | 4369 | C47 | PC | A | 778 | 74.121 | 34.185 | 27.662 | 1.00 | 42.80 |
| ATOM | 4370 | C48 | PC | A | 778 | 73.131 | 33.504 | 26.727 | 1.00 | 43.31 |
| ATOM | 4371 | C11 | PC | A | 778 | 86.101 | 31.542 | 30.412 | 1.00 | 71.27 |
| ATOM | 4372 | C12 | PC | A | 778 | 84.804 | 32.359 | 30.441 | 1.00 | 66.82 |
| ATOM | 4373 | C13 | PC | A | 778 | 83.700 | 31.845 | 29.548 | 1.00 | 59.12 |
| ATOM | 4374 | C14 | PC | A | 778 | 83.836 | 32.380 | 28.141 | 1.00 | 55.92 |
| ATOM | 4375 | C15 | PC | A | 778 | 82.896 | 31.671 | 27.184 | 1.00 | 53.76 |
| ATOM | 4376 | C16 | PC | A | 778 | 82.613 | 30.248 | 27.633 | 1.00 | 52.97 |
| ATOM | 4377 | C17 | PC | A | 778 | 83.562 | 29.250 | 26.990 | 1.00 | 50.51 |
| ATOM | 4378 | C18 | PC | A | 778 | 84.990 | 29.415 | 27.480 | 1.00 | 47.60 |
| ATOM | 4379 | C19 | PC | A | 778 | 85.860 | 30.046 | 26.408 | 1.00 | 47.69 |
| ATOM | 4380 | C20 | PC | A | 778 | 86.359 | 29.011 | 25.411 | 1.00 | 44.90 |
| ATOM | 4381 | C21 | PC | A | 778 | 85.271 | 28.598 | 24.439 | 1.00 | 44.44 |
| ATOM | 4382 | C22 | PC | A | 778 | 85.574 | 29.064 | 23.038 | 1.00 | 44.15 |
| ATOM | 4383 | C23 | PC | A | 778 | 86.167 | 30.453 | 23.044 | 1.00 | 43.99 |
| ATOM | 4384 | C24 | PC | A | 778 | 87.670 | 30.411 | 22.853 | 1.00 | 42.63 |
| ATOM | 4385 | C25 | PC | A | 778 | 88.068 | 31.161 | 21.599 | 1.00 | 43.28 |
| ATOM | 4386 | C26 | PC | A | 778 | 87.733 | 32.642 | 21.693 | 1.00 | 41.66 |
| ATOM | 4387 | C27 | PC | A | 778 | 88.852 | 33.492 | 21.119 | 1.00 | 40.33 |
| ATOM | 4388 | C28 | PC | A | 778 | 90.094 | 33.503 | 21.969 | 1.00 | 37.09 |
| ATOM | 4389 | O11 | PC | A | 778 | 86.692 | 31.253 | 29.343 | 1.00 | 72.85 |
| ATOM | 4390 | O31 | PC | A | 778 | 83.619 | 33.857 | 35.833 | 1.00 | 78.26 |
| ATOM | 4391 | O2 | PC | A | 778 | 85.652 | 33.065 | 34.823 | 1.00 | 81.22 |
| ATOM | 4392 | O3 | PC | A | 778 | 86.615 | 31.154 | 31.736 | 1.00 | 75.71 |
| ATOM | 4393 | O1P | PC | A | 778 | 89.401 | 31.823 | 36.960 | 1.00 | 100.00 |
| ATOM | 4394 | O2P | PC | A | 778 | 89.563 | 33.853 | 35.511 | 1.00 | 100.00 |
| ATOM | 4395 | O3P | PC | A | 778 | 88.125 | 31.917 | 34.809 | 1.00 | 96.24 |
| ATOM | 4396 | O4P | PC | A | 778 | 87.424 | 33.342 | 36.757 | 1.00 | 100.00 |
| ATOM | 4397 | P | PC | A | 778 | 88.628 | 32.739 | 36.008 | 1.00 | 100.00 |
| ATOM | 4398 | OH2 | HOH | A | 901 | 116.815 | 15.747 | 16.799 | 1.00 | 43.90 |
| ATOM | 4401 | OH2 | HOH | A | 902 | 107.439 | 22.033 | 22.778 | 1.00 | 44.99 |
| ATOM | 4404 | OH2 | HOH | A | 903 | 115.201 | 26.524 | 27.768 | 1.00 | 47.84 |
| ATOM | 4407 | OH2 | HOH | A | 904 | 83.653 | 23.737 | 13.286 | 1.00 | 22.80 |
| ATOM | 4410 | OH2 | HOH | A | 905 | 76.576 | 23.779 | 22.886 | 1.00 | 55.25 |
| ATOM | 4413 | OH2 | HOH | A | 906 | 101.110 | 43.595 | 19.882 | 1.00 | 44.52 |
| ATOM | 4416 | OH2 | HOH | A | 907 | 80.990 | 19.920 | 23.229 | 1.00 | 48.61 |
| ATOM | 4419 | OH2 | HOH | A | 908 | 141.374 | 35.733 | 18.039 | 1.00 | 56.87 |
| ATOM | 4422 | OH2 | HOH | A | 909 | 81.958 | 22.677 | 23.953 | 1.00 | 61.10 |
| ATOM | 4425 | OH2 | HOH | A | 910 | 152.059 | 36.697 | −0.688 | 1.00 | 62.06 |
| ATOM | 4428 | OH2 | HOH | A | 911 | 139.649 | 24.807 | 2.271 | 1.00 | 53.67 |
| ATOM | 4431 | OH2 | HOH | A | 912 | 124.594 | 31.177 | 0.772 | 1.00 | 55.59 |
| ATOM | 4434 | OH2 | HOH | A | 913 | 121.471 | 25.892 | 1.624 | 1.00 | 38.62 |
| ATOM | 4437 | OH2 | HOH | A | 914 | 114.402 | 25.412 | 0.380 | 1.00 | 31.16 |
| ATOM | 4440 | OH2 | HOH | A | 915 | 147.939 | 35.177 | −2.062 | 1.00 | 45.61 |
| ATOM | 4443 | OH2 | HOH | A | 916 | 74.995 | 34.193 | 14.634 | 1.00 | 73.76 |
| ATOM | 4446 | OH2 | HOH | A | 917 | 105.633 | 17.580 | 14.444 | 1.00 | 37.72 |
| ATOM | 4449 | OH2 | HOH | A | 918 | 71.679 | 28.574 | 37.701 | 1.00 | 51.80 |
| ATOM | 4452 | OH2 | HOH | A | 919 | 104.762 | 14.189 | 10.052 | 1.00 | 57.21 |
| ATOM | 4455 | OH2 | HOH | A | 920 | 80.378 | 28.515 | 41.681 | 1.00 | 81.97 |
| ATOM | 4458 | OH2 | HOH | A | 921 | 80.215 | 25.918 | 35.227 | 1.00 | 29.12 |
| ATOM | 4461 | OH2 | HOH | A | 922 | 79.054 | 35.414 | 40.975 | 1.00 | 41.95 |
| ATOM | 4464 | OH2 | HOH | A | 923 | 65.692 | 27.690 | 51.372 | 1.00 | 65.22 |
| ATOM | 4467 | OH2 | HOH | A | 924 | 88.914 | 24.630 | 4.689 | 1.00 | 48.96 |
| ATOM | 4470 | OH2 | HOH | A | 925 | 100.082 | 22.695 | 34.405 | 1.00 | 48.58 |
| ATOM | 4473 | OH2 | HOH | A | 926 | 76.929 | 19.455 | 31.644 | 1.00 | 86.93 |
| ATOM | 4476 | OH2 | HOH | A | 927 | 171.524 | 31.723 | 13.880 | 1.00 | 59.94 |
| ATOM | 4479 | OH2 | HOH | A | 928 | 108.006 | 35.100 | 24.514 | 1.00 | 49.31 |
| ATOM | 4482 | OH2 | HOH | A | 929 | 141.049 | 21.649 | 5.587 | 1.00 | 42.12 |
| ATOM | 4485 | OH2 | HOH | A | 930 | 110.883 | 40.970 | 11.363 | 1.00 | 35.61 |
| ATOM | 4488 | OH2 | HOH | A | 931 | 74.360 | 34.525 | 47.158 | 1.00 | 75.14 |
| ATOM | 4491 | OH2 | HOH | A | 932 | 68.751 | 40.894 | 44.809 | 1.00 | 43.22 |
| ATOM | 4494 | OH2 | HOH | A | 933 | 59.758 | 29.313 | 23.460 | 1.00 | 61.69 |
| ATOM | 4497 | OH2 | HOH | A | 934 | 95.173 | 16.341 | 20.293 | 1.00 | 48.05 |
| ATOM | 4500 | OH2 | HOH | A | 935 | 111.623 | 41.720 | 8.640 | 1.00 | 53.02 |
| ATOM | 4503 | OH2 | HOH | A | 936 | 105.604 | 16.201 | 11.554 | 1.00 | 71.57 |
| ATOM | 4506 | OH2 | HOH | A | 937 | 97.160 | 30.542 | 3.465 | 1.00 | 58.62 |
| ATOM | 4509 | OH2 | HOH | A | 938 | 108.492 | 10.703 | 6.225 | 1.00 | 76.16 |
| ATOM | 4512 | OH2 | HOH | A | 939 | 135.408 | 44.678 | 13.765 | 1.00 | 61.58 |
| ATOM | 4515 | OH2 | HOH | A | 940 | 91.469 | 31.723 | 41.685 | 1.00 | 41.59 |
| ATOM | 4518 | OH2 | HOH | A | 941 | 164.580 | 32.799 | 18.975 | 1.00 | 48.97 |
| ATOM | 4521 | OH2 | HOH | A | 942 | 157.888 | 29.985 | 20.509 | 1.00 | 63.61 |
| ATOM | 4524 | OH2 | HOH | A | 943 | 121.776 | 21.112 | 25.392 | 1.00 | 61.93 |
| ATOM | 4527 | OH2 | HOH | A | 944 | 96.503 | 38.299 | 34.009 | 1.00 | 64.71 |
| ATOM | 4530 | OH2 | HOH | A | 945 | 96.403 | 17.036 | 28.115 | 1.00 | 76.51 |
| ATOM | 4533 | OH2 | HOH | A | 946 | 88.114 | 18.204 | 31.407 | 1.00 | 52.33 |
| ATOM | 4536 | OH2 | HOH | A | 947 | 81.217 | 23.098 | 12.608 | 1.00 | 35.93 |
| ATOM | 4539 | OH2 | HOH | A | 948 | 126.713 | 24.917 | 0.514 | 1.00 | 82.50 |
| TER | | | | | | | | | | |
| END | | | | | | | | | | |

| | |
|---|---|
| Atom | |
| Type | Residue # X Y Z OCC B |

| | | | | |
|---|---|---|---|---|
| REMARK | 3 | | | |
| REMARK | 3 | REFINEMENT. | | |
| REMARK | 3 | PROGRAM | X-PLOR (online) | 3.843 |
| REMARK | 3 | AUTHORS | BRUNGER | |
| REMARK | 3 | | | |
| REMARK | 3 | DATA USED IN REFINEMENT. | | |
| REMARK | 3 | RESOLUTION RANGE HIGH (ANGSTROMS) | 2.40 | |
| REMARK | 3 | RESOLUTION RANGE LOW (ANGSTROMS) | 50.00 | |
| REMARK | 3 | DATA CUTOFF (SIGMA (F)) | 0.0 | |
| REMARK | 3 | DATA CUTOFF HIGH (ABS (F)) | 100000.00 | |
| REMARK | 3 | DATA CUTOFF LOW (ABS (F)) | 0.010000 | |
| REMARK | 3 | COMPLETENESS (WORKING + TEST) (%) | 92.7 | |
| REMARK | 3 | NUMBER OF REFLECTIONS | 18908 | |
| REMARK | 3 | | | |
| REMARK | 3 | FIT TO DATA USED IN REFINEMENT. | | |
| REMARK | 3 | CROSS-VALIDATION METHOD | THROUGHOUT | |
| REMARK | 3 | FREE R VALUE TEST SET SELECTION | RANDOM | |
| REMARK | 3 | R VALUE (WORKING SET) | 0.225 | |
| REMARK | 3 | FREE R VALUE | 0.295 | |
| REMARK | 3 | FREE R VALUE TEST SET SIZE (%) | 10.2 | |
| REMARK | 3 | FREE R VALUE TEST SET COUNT | 1926 | |
| REMARK | 3 | ESTIMATED ERROR OF FREE R VALUE | 0.007 | |
| REMARK | 3 | | | |
| REMARK | 3 | FIT IN THE HIGHEST RESOLUTION BIN. | | |
| REMARK | 3 | TOTAL NUMBER OF BINS USED | 6 | |
| REMARK | 3 | BIN RESOLUTION RANGE HIGH (A) | 2.40 | |
| REMARK | 3 | BIN RESOLUTION RANGE LOW (A) | 2.55 | |
| REMARK | 3 | BIN COMPLETENESS (WORKING + TEST) (%) | 94.1 | |
| REMARK | 3 | REFLECTIONS IN BIN (WORKING SET) | 2875 | |
| REMARK | 3 | BIN R VALUE (WORKING SET) | 0.368 | |
| REMARK | 3 | BIN FREE R VALUE | 0.455 | |
| REMARK | 3 | BIN FREE R VALUE TEST SET SIZE (%) | 8.7 | |
| REMARK | 3 | BIN FREE R VALUE TEST SET COUNT | 275 | |
| REMARK | 3 | ESTIMATED ERROR OF BIN FREE R VALUE | 0.027 | |
| REMARK | 3 | | | |
| REMARK | 3 | NUMBER OF NON-HYDROGEN ATOMS USED IN REFINEMENT. | | |
| REMARK | 3 | PROTEIN ATOMS | 3531 | |
| REMARK | 3 | NUCLEIC ACID ATOMS | 0 | |
| REMARK | 3 | HETEROGEN ATOMS | 102 | |
| REMARK | 3 | SOLVENT ATOMS | 48 | |
| REMARK | 3 | | | |
| REMARK | 3 | B VALUES. | | |
| REMARK | 3 | FROM WILSON PLOT (A**2) | 45.0 | |
| REMARK | 3 | MEAN B VALUE (OVERALL, A**2) | 45.0 | |
| REMARK | 3 | OVERALL ANISOTROPIC B VALUE. | | |
| REMARK | 3 | B11 (A**2) | −11.35 | |
| REMARK | 3 | B22 (A**2) | 6.33 | |
| REMARK | 3 | B33 (A**2) | 5.02 | |
| REMARK | 3 | B12 (A**2) | 0.00 | |
| REMARK | 3 | B13 (A**2) | 7.70 | |
| REMARK | 3 | B23 (A**2) | 0.00 | |
| REMARK | 3 | | | |
| REMARK | 3 | ESTIMATED COORDINATE ERROR. | | |
| REMARK | 3 | ESD FROM LUZZATI PLOT (A) | 0.34 | |
| REMARK | 3 | ESD FROM SIGMAA (A) | 0.47 | |
| REMARK | 3 | LOW RESOLUTION CUTOFF (A) | 5.00 | |
| REMARK | 3 | | | |
| REMARK | 3 | CROSS-VALIDATED ESTIMATED COORDINATE ERROR. | | |
| REMARK | 3 | ESD FROM C-V LUZZATI PLOT (A) | 0.45 | |
| REMARK | 3 | ESD FROM C-V SIGMAA (A) | 0.46 | |
| REMARK | 3 | | | |
| REMARK | 3 | RMS DEVIATIONS FROM IDEAL VALUES. | | |
| REMARK | 3 | BOND LENGTHS (A) | 0.007 | |
| REMARK | 3 | BOND ANGLES (DEGREES) | 1.4 | |
| REMARK | 3 | DIHEDRAL ANGLES (DEGREES) | 26.1 | |
| REMARK | 3 | IMPROPER ANGLES (DEGREES) | 1.24 | |
| REMARK | 3 | | | |
| REMARK | 3 | | | |

| | | | | |
|---|---|---|---|---|
| REMARK | 3 | ISOTROPIC THERMAL FACTOR RESTRAINTS. | RMS | SIGMA |
| REMARK | 3 | MAIN-CHAIN BOND (A**2) | 1.77; | 1.50 |
| REMARK | 3 | MAIN-CHAIN ANGLE (A**2) | 3.00; | 2.00 |
| REMARK | 3 | SIDE-CHAIN BOND (A**2) | 2.81; | 2.00 |
| REMARK | 3 | SIDE-CHAIN ANGLE (A**2) | 4.53; | 2.50 |
| REMARK | 3 | | | |

| | | | | |
|---|---|---|---|---|
| REMARK | 3 | NCS MODEL | NONE | |
| REMARK | 3 | | | |
| REMARK | 3 | NCS RESTRAINTS. | RMS | SIGMA/WEIGHT |
| REMARK | 3 | GROUP 1 POSITIONAL (A) | NULL; | NULL |
| REMARK | 3 | GROUP 1 B-FACTOR (A**2) | NULL; | NULL |
| REMARK | 3 | | | |

| REMARK | 3 | PARAMETER FILE 1 | | | | parhcsdx.pro | | | | | | | | | | |
|---|---|---|---|---|---|---|---|---|---|---|---|---|---|---|---|---|
| REMARK | 3 | PARAMETER FILE 2 | | | | paramll.wat | | | | | | | | | | |
| REMARK | 3 | TOPOLOGY FILE 1 | | | | tophcsdx.pro | | | | | | | | | | |
| REMARK | 3 | TOPOLOGY FILE 2 | | | | tophll.wat | | | | | | | | | | |
| REMARK | 3 | | | | | | | | | | | | | | | |
| REMARK | 3 | OTHER REFINEMENT REMARKS | | | | | | BULK SOLVENT MODEL USED | | | | | | | | |
| SEQRES | 1 | A | 507 | VAL | ASN | PRO | GLY | VAL | VAL | VAL | ARG | ILE | SER | GLN | LYS | GLY |
| SEQRES | 2 | A | 507 | LEU | ASP | TYR | ALA | SER | GLN | GLN | GLY | THR | ALA | ALA | LEU | GLN |
| SEQRES | 3 | A | 507 | LYS | GLU | LEU | LYS | ARG | ILE | LYS | ILE | PRO | ASP | TYR | SER | ASP |
| SEQRES | 4 | A | 507 | SER | PHE | LYS | ILE | LYS | HIS | LEU | GLY | LYS | GLY | HIS | TYR | SER |
| SEQRES | 5 | A | 507 | PHE | TYR | SER | MET | ASP | ILE | ARG | GLU | PHE | GLN | LEU | PRO | SER |
| SEQRES | 6 | A | 507 | SER | GLN | ILE | SER | MET | VAL | PRO | ASN | VAL | GLY | LEU | LYS | PHE |
| SEQRES | 7 | A | 507 | SER | ILE | SER | ASN | ALA | ASN | ILE | LYS | ILE | SER | GLY | LYS | TRP |
| SEQRES | 8 | A | 507 | LYS | ALA | GLN | LYS | ARG | PHE | LEU | LYS | MET | SER | GLY | ASN | PHE |
| SEQRES | 9 | A | 507 | ASP | LEU | SER | ILE | GLU | GLY | MET | SER | ILE | SER | ALA | ASP | LEU |
| SEQRES | 10 | A | 507 | LYS | LEU | GLY | SER | ASN | PRO | THR | SER | GLY | LYS | PRO | THR | ILE |
| SEQRES | 11 | A | 507 | THR | CYS | SER | SER | CYS | SER | SER | HIS | ILE | ASN | SER | VAL | HIS |
| SEQRES | 12 | A | 507 | VAL | HIS | ILE | SER | ALA | ALA | SER | VAL | GLY | TRP | LEU | ILE | GLN |
| SEQRES | 13 | A | 507 | LEU | PHE | HIS | LYS | LYS | ILE | GLU | SER | ALA | LEU | ARG | ASN | LYS |
| SEQRES | 14 | A | 507 | MET | ASN | SER | GLN | VAL | CYS | GLU | LYS | VAL | THR | ASN | SER | VAL |
| SEQRES | 15 | A | 507 | SER | SER | GLU | LEU | GLN | PRO | TYR | PHE | GLN | THR | LEU | PRO | VAL |
| SEQRES | 16 | A | 507 | MET | THR | LYS | ILE | ASP | SER | VAL | ALA | GLY | ILE | ASN | TYR | GLY |
| SEQRES | 17 | A | 507 | LEU | VAL | ALA | PRO | PRO | ALA | THR | THR | ALA | GLU | THR | LEU | ASP |
| SEQRES | 18 | A | 507 | VAL | GLN | MET | LYS | GLY | GLU | PHE | TYR | SER | GLU | ALA | ALA | ALA |
| SEQRES | 19 | A | 507 | ALA | PRO | PRO | PRO | PHE | ALA | PRO | PRO | VAL | MET | GLU | PHE | PRO |
| SEQRES | 20 | A | 507 | ALA | ALA | ALA | ASP | ARG | MET | VAL | TYR | LEU | GLY | LEU | SER | ASP |
| SEQRES | 21 | A | 507 | TYR | PHE | PHE | ASN | THR | ALA | GLY | LEU | VAL | TYR | GLN | GLU | ALA |
| SEQRES | 22 | A | 507 | GLY | VAL | LEU | LYS | MET | THR | LEU | ARG | ASP | ASP | MET | ILE | PRO |
| SEQRES | 23 | A | 507 | LYS | GLU | SER | ALA | PHE | ARG | LEU | THR | THR | SER | PHE | PHE | GLY |
| SEQRES | 24 | A | 507 | THR | PHE | LEU | PRO | GLU | VAL | ALA | LYS | LYS | PHE | PRO | ASN | MET |
| SEQRES | 25 | A | 507 | LYS | ILE | GLN | ILE | HIS | VAL | SER | ALA | SER | THR | PRO | PRO | HIS |
| SEQRES | 26 | A | 507 | LEU | SER | VAL | GLN | PRO | THR | GLY | LEU | THR | PHE | TYR | PRO | ALA |
| SEQRES | 27 | A | 507 | VAL | ASP | VAL | GLN | ALA | PHE | ALA | VAL | LEU | PRO | ASN | SER | ALA |
| SEQRES | 28 | A | 507 | LEU | ALA | SER | LEU | PHE | LEU | ILE | GLY | MET | HIS | THR | THR | GLY |
| SEQRES | 29 | A | 507 | SER | MET | GLU | VAL | SER | ALA | GLU | SER | ASN | ARG | LEU | VAL | GLY |
| SEQRES | 30 | A | 507 | GLU | LEU | LYS | LEU | ASP | ARG | LEU | LEU | LEU | GLU | LEU | LYS | HIS |
| SEQRES | 31 | A | 507 | SER | ASN | ILE | GLY | PRO | PHE | PRO | VAL | GLU | LEU | LEU | GLN | ASP |
| SEQRES | 32 | A | 507 | ILE | MET | ASN | TYR | ILE | VAL | PRO | ILE | LEU | VAL | LEU | PRO | ARG |
| SEQRES | 33 | A | 507 | VAL | ASN | GLU | LYS | LEU | GLN | LYS | GLY | PHE | PRO | LEU | PRO | THR |
| SEQRES | 34 | A | 507 | PRO | ALA | ARG | VAL | GLN | LEU | TYR | ASN | VAL | VAL | LEU | GLN | PRO |
| SEQRES | 35 | A | 507 | HIS | GLN | ASN | PHE | LEU | LEU | PHE | GLY | ALA | ASP | VAL | VAL | TYR |
| SEQRES | 36 | A | 507 | LYS | PC | PC | HOH | HOH | HOH | HOH | HOH | HOH | HOH | HOH | HOH | HOH |
| SEQRES | 37 | A | 507 | HOH | HOH | HOH | HOH | HOH | HOH | HOH | HOH | HOH | HOH | HOH | HOH | HOH |
| SEQRES | 38 | A | 507 | HOH | HOH | HOH | HOH | HOH | HOH | HOH | HOH | HOH | HOH | HOH | HOH | HOH |
| SEQRES | 39 | A | 507 | HOH | HOH | HOH | HOH | HOH | HOH | HOH | HOH | HOH | HOH | HOH | HOH | HOH |

SSBOND 1 CYS A 135 CYS A 175
CRYST1 185.600 33.000 85.200 90.00 101.60 90.00 C 2 4

| | | | | |
|---|---|---|---|---|
| ORIGX1 | 1.000000 | 0.000000 | 0.000000 | 0.00000 |
| ORIGX2 | 0.000000 | 1.000000 | 0.000000 | 0.00000 |
| ORIGX3 | 0.000000 | 0.000000 | 1.000000 | 0.00000 |
| SCALE1 | 0.005388 | 0.000000 | 0.001106 | 0.00000 |
| SCALE2 | 0.000000 | 0.030303 | 0.000000 | 0.00000 |
| SCALE3 | 0.000000 | 0.000000 | 0.011982 | 0.00000 |

The following abbreviations are used in Table 4 in accordance with the format and usage established by the Protein Data Bank (“PDB”), Brookhaven National Laboratory, Brookhaven, N.Y. These coordinates are on deposit with the PDB, ID code 1bp1. Atomic coordinates of a BPI protein as described herein appear at Table 4 (pages 62-171 herein) and refinement statistics also appear at the end of Table 4 (pages 172-173 herein). Table 4 corresponds to FIG. 6 (FIGS. 6.1-6.112) in U.S. patent application Ser. No. 08/879,565, filed Jun. 20, 1997.

“Atom type” refers to the element whose coordinates are measured. The first letter in the column defines the element.

“Residue” refers to the amino acid in the BPI protein sequence, using the standard three letter abbreviations known in the art.

“#” refers to the residue number.

“X, Y, Z” crystallographically define the atomic position, in three-dimensional space, of the element measured.

“OCC” is the occupancy value.

“B” is a thermal factor that measures movement of the atom around its atomic center.

10. Organomimetics

Molecular modelling of BPI as described herein is useful for the preparation of organomimetics such as “surface” mimetics. As one example, organomimetics are prepared based on “tip” mimetics in which the three-dimensional coordinates of the tip, as described above, are used to create a “surface” (or complementary pocket) into which a computer program builds an organic molecule with similar characteristics.

### EXAMPLE 4

### Stnrcture Determination of a Crystallized BPI Protein at 1.7 Å Resolution

Additional studies were performed to extend the resolution of the crystal structure of a BPI protein beyond the 2.4 Å resolution described in Example 2. For these studies, a sample of BPI protein was crystallized and the crystal structure was determined at a resolution of 1.7 Å using cryo-crystallography diffraction data. This crystal structure was compared to the crystal structure of 2.4 Å resolution at room temperature as described in Example 2. An additional exemplary set of structure coordinates for a BPI protein are listed below in Table 6.

Crystals of recombinant human BPI were grown generally as described in Example 2. Specifically, crystals were equilibrated over-night to 25% (v/v) PEG 6000 by vapor diffusion, and then immediately into 45% PEG 6000 for approximately two minutes. The crystals were then mounted and frozen under a stream of liquid nitrogen.

X-ray diffraction data for these crystallized BPI were collected at the Brookhaven National Laboratory on beamline X-12B. Data were processed and scaled using the programs DENZO and SCALEPACK [Z. Otwinowski, in Proceedings of CCP4 Study Weekend: Data Collection and Processing, L. Sawyer, N. Isaacs, S. Balleys, Eds. (SERC Daresbury Laboratory, Warrington, UK, 1993) pp. 56-62].

The unit cell dimensions of frozen crystals differ from the room temperature crystal dimensions as described in Example 2, especially the b-axis which contracted 16%. Rigid-body refinement in X-PLOR [A. Brünger, et al., ActaCrystallog. Sect. D, 54:905 (1998)] was used to reorient the room-temperature BPI model (protein atoms only) in the smaller unit cell, followed by simulated annealing and individual B-factor refinement. This model had an R-factor of 28.4% and R_{free} of 34.3%. Electron density for two molecules of phosphatidylcholine, each located in an apolar pocket in both domains of BPI, was apparent in both 2Fₒ-F_{c} and Fₒ-F_{c} maps. Both lipids were also found during tracing of the original model, and likely were introduced to BPI during protein purification. Modeling of the N-terminal phospholipid reduced the R-factor to 27.3%, and R_{free} to 33.5%. Approximately 360 water molecules and the C-terminal phospholipid (excluding the disordered head) were gradually introduced into the model and refined to an R-factor of 21.3% and R_{free} of 29.6%.

Once the R-factors could not be lowered using X-PLOR, the program CNS was used, which uses a maximum-likelihood target function to minimize the gap between R and R_{free} [A. Brünger, et al., Acta Crystallog. Sect. D, 54:905 (1998)]. A noticeable improvement in the quality of the electron density maps was observed upon using CNS, which allowed for further rebuilding the model. Following a bulk-solvent and overall anisotropic B-factor correction, the final R-factor was 19.8% and final R_{free} was 24.9%. Refinement statistics are summarized in Table 5.

TABLE 5
STATISTICS ON CRYSTALLOGRAPHIC
DATA AND REFINEMENT FOR BPI

| A. Data Collection | |
|---|---|
| Resolution range high (Å) | 1.7 |
| Resolution range low (Å) | 50.0 |
| Data cut-off (Sigma (F)) | 0.0 |
| Overall completeness | 94.1 (77.2) |
| (final res. shell) | |
| R_{merge} (%) | 4.9 |
| Redundancy | 2.3 |
| Number of reflections (test set) | 4755 |
| Number of reflections (overall) | 47,197 |
| B. Crystal | |
| Spacegroup | C2 |
| Unit cell parameters (Å²) | a = 184.3, b = 31.2, c = 80.6, |
| | β = 103.2 |
| C. Refinement | |
| R-value (%) | 19.8 |
| Free R-value (%) | 24.9 |
| Mean B-value (all atoms) (Å²) | 29.8 |
| Estimated coordinate error | 0.20 |
| (Luzzati) (Å) | |
| rmsd from ideal values | |
| Bond lengths (Å) | 0.017 |
| Bond angles (deg.) | 2.0 |
| Dihedral angles (deg.) | 25.9 |
| Improper angels (deg.) | 1.33 |

The following residues had weak density and therefore were not included in the refinement: K33, K44, K45, K86, K95, K118, K290, K307, K313. Multiple conformations were built for the following residues: Q26, D57, S79, S88, S107, H143, T179, S183, V243, H325, L326, T363, N373, R374, F425, N437, V453, V454.

Several structure validation methods were used to assess the quality of the high-resolution BPI model. No significant errors were found in the model using the programs VERIFY 3D, ERRAT, PROCHECK and WHAT IF (C. Colovos and T. Yeates, Protein Science, 2:1511-19 (1993); R. A. Lackowski et al., J. Appi. Crystallog. 26:283-291 (1993); R. Lüthy et al., Nature 356:83-85 (1992)]. Over 89% of the residues are in the most favorable position in a Ramachandran plot. Approximately 10% of the residues are in the additionally allowed regions. Composite, simulated-annealing omit maps were calculated for the entire molecule. Density from omit maps agreed very well with the atomic positions of nearly all residues.

Comparison of the higher resolution model of BPI, presented in this Example 4, with the room temperature model presented in Example 2 reveals little structural change, with the exception of residues 42 to 48. In the new structure, several side-chains on one side of the loop now pack against the protein, whereas these side chains in previous model were mostly exposed to solvent. The loop rearrangement may be due to conditioning the crystals with 45% PEG 6000 for cryo-protection, freezing, or a combination of the two. Equivalent main-chain atoms for the two models superimpose with an rmsd of 0.9 Å.

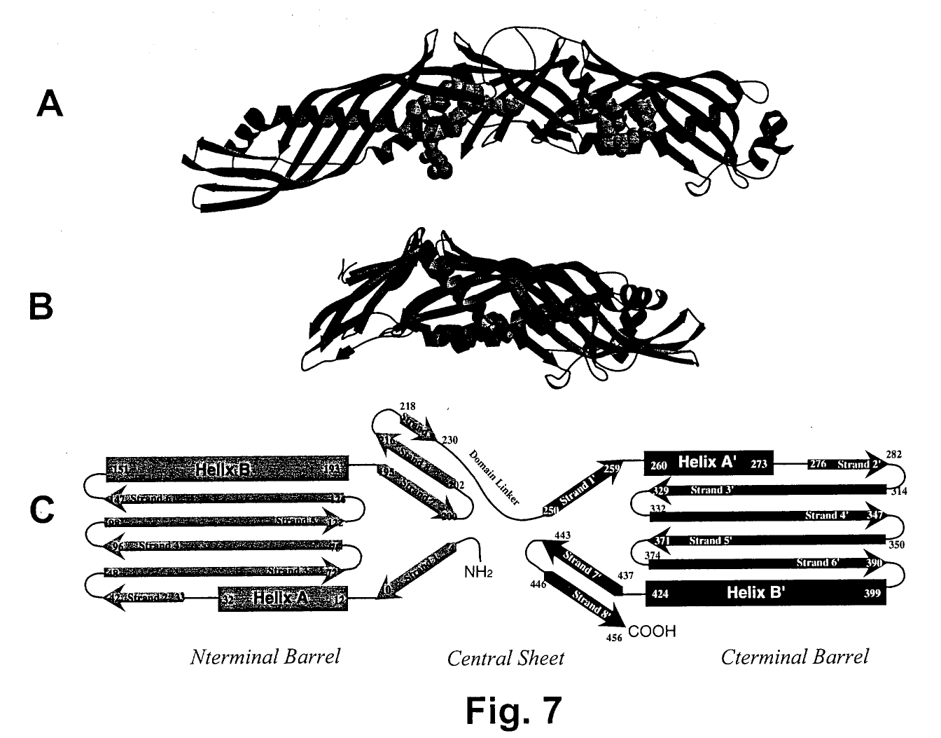

FIG. 7(a) is a ribbon representation of the 1.7 Å crystal structure of human BPI, with an N-terminal domain (blue) and a C-terminal domain (red). As described in Example 2, and FIG. 1, residues 10 to 193 fold into a structural element called the N-terminal barrel. The barrel is composed of five anti-parallel β-strands which twist about the barrel axis. Two α-helices complete the barrel by closing a gap in the β-sheet. Residues 260 to 430 fold into a similar structure called the C-terminal barrel. Amino acid residues 201 to 229, as well as 431 to 456, fold into the central β-sheet of six strands, located in the center of the molecule, which interacts with both the N-terminal and C-terminal barrels. A linker of residues 230 to 250 (olive) connects the N-terminal and C-terminal domains. FIG. 7(b) shows the superposition of the N-terminal domain (blue) on the C-terminal domain (red). Residues 1-229 were structurally aligned to residues 251-456 using the algorithm ALIGN_V2 [Cohen, et al., J. Mol. Biol. 190:593-604 1986]. the two domains align with 3.0 Å root mean square deviation over the main-chain atoms of the 173 structurally corresponding residues. FIG. 7(c) is a schematic of BPI showing its elongated shape and two-domain structure. The two domains are related by a pseudodyad perpendicular to the page. Secondary structure units are represented by arrows (β-strands) and rectangles (helices). The N-terminal domain (residues 1-229) is gray; the C-terminal domain (residues 251 to 456) is black. Secondary structure units have been numbered, with the primes denoting the units in the C-terminal domain. Residue positions for the start and end of each secondary structure unit are shown. The three subdomains (N-terminal barrel, C-terminal barrel, and central sheet) are shown.

TABLE 6

| | | | | | | | | | | | | |
|---|---|---|---|---|---|---|---|---|---|---|---|---|
| ATOM | 1 | N | VAL | A | 1 | 99.123 | 12.600 | 15.157 | 1.00 | 44.04 | N | |
| ATOM | 2 | CA | VAL | A | 1 | 98.694 | 12.677 | 13.729 | 1.00 | 40.54 | C | |
| ATOM | 3 | C | VAL | A | 1 | 97.859 | 13.917 | 13.453 | 1.00 | 38.35 | C | |
| ATOM | 4 | O | VAL | A | 1 | 98.159 | 14.635 | 12.519 | 1.00 | 38.36 | O | |
| ATOM | 5 | CB | VAL | A | 1 | 97.903 | 11.413 | 13.319 | 1.00 | 44.86 | C | |
| ATOM | 6 | N | ASN | A | 2 | 96.797 | 14.144 | 14.233 | 1.00 | 34.30 | N | |
| ATOM | 7 | CA | ASN | A | 2 | 95.951 | 15.329 | 14.047 | 1.00 | 33.52 | C | |
| ATOM | 8 | C | ASN | A | 2 | 96.045 | 16.262 | 15.254 | 1.00 | 28.68 | C | |
| ATOM | 9 | O | ASN | A | 2 | 95.362 | 16.081 | 16.275 | 1.00 | 31.83 | O | |
| ATOM | 10 | CB | ASN | A | 2 | 94.496 | 14.946 | 13.830 | 1.00 | 38.28 | C | |
| ATOM | 11 | CG | ASN | A | 2 | 94.295 | 14.156 | 12.538 | 1.00 | 45.11 | C | |
| ATOM | 12 | OD1 | ASN | A | 2 | 93.178 | 13.746 | 12.200 | 1.00 | 50.87 | O | |
| ATOM | 13 | ND2 | ASN | A | 2 | 95.384 | 13.946 | 11.811 | 1.00 | 47.51 | N | |
| ATOM | 14 | N | PRO | A | 3 | 96.933 | 17.237 | 15.177 | 1.00 | 23.64 | N | |
| ATOM | 15 | CA | PRO | A | 3 | 97.026 | 18.145 | 16.319 | 1.00 | 22.44 | C | |
| ATOM | 16 | C | PRO | A | 3 | 95.864 | 19.158 | 16.345 | 1.00 | 18.47 | C | |
| ATOM | 17 | O | PRO | A | 3 | 95.165 | 19.342 | 15.339 | 1.00 | 20.64 | O | |
| ATOM | 18 | CB | PRO | A | 3 | 98.372 | 18.828 | 16.086 | 1.00 | 23.56 | C | |
| ATOM | 19 | CG | PRO | A | 3 | 98.439 | 18.928 | 14.576 | 1.00 | 23.16 | C | |
| ATOM | 20 | CD | PRO | A | 3 | 98.008 | 17.490 | 14.207 | 1.00 | 25.78 | C | |
| ATOM | 21 | N | GLY | A | 4 | 95.701 | 19.803 | 17.500 | 1.00 | 19.55 | N | |
| ATOM | 22 | CA | GLY | A | 4 | 94.713 | 20.880 | 17.661 | 1.00 | 18.57 | C | |
| ATOM | 23 | C | GLY | A | 4 | 95.225 | 22.175 | 17.010 | 1.00 | 18.22 | C | |
| ATOM | 24 | O | GLY | A | 4 | 94.427 | 23.044 | 16.520 | 1.00 | 18.00 | O | |
| ATOM | 25 | N | VAL | A | 5 | 96.545 | 22.311 | 16.979 | 1.00 | 16.24 | N | |
| ATOM | 26 | CA | VAL | A | 5 | 97.203 | 23.505 | 16.429 | 1.00 | 16.48 | C | |
| ATOM | 27 | C | VAL | A | 5 | 98.475 | 23.082 | 15.680 | 1.00 | 20.26 | C | |
| ATOM | 28 | O | VAL | A | 5 | 99.229 | 22.213 | 16.168 | 1.00 | 17.97 | O | |
| ATOM | 29 | CB | VAL | A | 5 | 97.610 | 24.453 | 17.580 | 1.00 | 15.95 | C | |
| ATOM | 30 | CG1 | VAL | A | 5 | 98.639 | 25.522 | 17.128 | 1.00 | 18.07 | C | |
| ATOM | 31 | CG2 | VAL | A | 5 | 96.371 | 25.119 | 18.119 | 1.00 | 15.07 | C | |
| ATOM | 32 | N | VAL | A | 6 | 98.688 | 23.657 | 14.497 | 1.00 | 17.00 | N | |
| ATOM | 33 | CA | VAL | A | 6 | 99.937 | 23.344 | 13.755 | 1.00 | 20.95 | C | |
| ATOM | 34 | C | VAL | A | 6 | 100.655 | 24.631 | 13.408 | 1.00 | 21.83 | C | |
| ATOM | 35 | O | VAL | A | 6 | 100.010 | 25.634 | 13.120 | 1.00 | 20.53 | O | |
| ATOM | 36 | CB | VAL | A | 6 | 99.670 | 22.482 | 12.472 | 1.00 | 22.17 | C | |
| ATOM | 37 | CG1 | VAL | A | 6 | 98.812 | 23.249 | 11.456 | 1.00 | 25.15 | C | |
| ATOM | 38 | CG2 | VAL | A | 6 | 100.994 | 22.096 | 11.802 | 1.00 | 22.54 | C | |
| ATOM | 39 | N | VAL | A | 7 | 101.990 | 24.614 | 13.507 | 1.00 | 23.46 | N | |
| ATOM | 40 | CA | VAL | A | 7 | 102.813 | 25.761 | 13.117 | 1.00 | 22.32 | C | |
| ATOM | 41 | C | VAL | A | 7 | 103.700 | 25.203 | 11.996 | 1.00 | 22.10 | C | |
| ATOM | 42 | O | VAL | A | 7 | 104.494 | 24.260 | 12.214 | 1.00 | 23.46 | O | |
| ATOM | 43 | CB | VAL | A | 7 | 103.722 | 26.281 | 14.255 | 1.00 | 24.50 | C | |
| ATOM | 44 | CG1 | VAL | A | 7 | 104.701 | 27.310 | 13.712 | 1.00 | 26.73 | C | |
| ATOM | 45 | CG2 | VAL | A | 7 | 102.866 | 26.942 | 15.339 | 1.00 | 26.51 | C | |
| ATOM | 46 | N | ARG | A | 8 | 103.496 | 25.740 | 10.794 | 1.00 | 19.55 | N | |
| ATOM | 47 | CA | ARG | A | 8 | 104.251 | 25.357 | 9.629 | 1.00 | 19.01 | C | |
| ATOM | 48 | C | ARG | A | 8 | 105.253 | 26.462 | 9.288 | 1.00 | 18.98 | C | |
| ATOM | 49 | O | ARG | A | 8 | 104.859 | 27.620 | 9.075 | 1.00 | 22.70 | O | |
| ATOM | 50 | CB | ARG | A | 8 | 103.306 | 25.141 | 8.454 | 1.00 | 23.41 | C | |
| ATOM | 51 | CG | ARG | A | 8 | 104.005 | 25.153 | 7.129 | 1.00 | 30.34 | C | |
| ATOM | 52 | CD | ARG | A | 8 | 103.055 | 24.724 | 6.049 | 1.00 | 36.53 | C | |
| ATOM | 53 | NE | ARG | A | 8 | 102.519 | 23.382 | 6.289 | 1.00 | 33.62 | N | |
| ATOM | 54 | CZ | ARG | A | 8 | 101.732 | 22.752 | 5.424 | 1.00 | 36.64 | C | |
| ATOM | 55 | NH1 | ARG | A | 8 | 101.415 | 23.362 | 4.281 | 1.00 | 37.02 | N | |
| ATOM | 56 | NH2 | ARG | A | 8 | 101.273 | 21.534 | 5.702 | 1.00 | 32.05 | N | |
| ATOM | 57 | N | ILE | A | 9 | 106.537 | 26.124 | 9.262 | 1.00 | 17.75 | N | |
| ATOM | 58 | CA | ILE | A | 9 | 107.562 | 27.105 | 8.903 | 1.00 | 20.58 | C | |
| ATOM | 59 | C | ILE | A | 9 | 107.946 | 26.883 | 7.419 | 1.00 | 23.60 | C | |
| ATOM | 60 | O | ILE | A | 9 | 108.258 | 25.756 | 7.005 | 1.00 | 19.72 | O | |
| ATOM | 61 | CB | ILE | A | 9 | 108.797 | 26.966 | 9.824 | 1.00 | 20.29 | C | |
| ATOM | 62 | CG1 | ILE | A | 9 | 108.329 | 27.010 | 11.291 | 1.00 | 20.75 | C | |
| ATOM | 63 | CG2 | ILE | A | 9 | 109.827 | 28.051 | 9.510 | 1.00 | 18.57 | C | |
| ATOM | 64 | CD1 | ILE | A | 9 | 109.409 | 26.612 | 12.300 | 1.00 | 24.49 | C | |
| ATOM | 65 | N | SER | A | 10 | 107.875 | 27.965 | 6.625 | 1.00 | 19.29 | N | |
| ATOM | 66 | CA | SER | A | 10 | 108.174 | 27.924 | 5.201 | 1.00 | 19.13 | C | |
| ATOM | 67 | C | SER | A | 10 | 109.674 | 28.141 | 4.963 | 1.00 | 19.57 | C | |
| ATOM | 68 | O | SER | A | 10 | 110.418 | 28.494 | 5.901 | 1.00 | 21.31 | O | |
| ATOM | 69 | CB | SER | A | 10 | 107.408 | 29.036 | 4.432 | 1.00 | 20.80 | C | |
| ATOM | 70 | OG | SER | A | 10 | 107.972 | 30.335 | 4.675 | 1.00 | 22.77 | O | |
| ATOM | 71 | N | GLN | A | 11 | 110.092 | 27.979 | 3.710 | 1.00 | 17.69 | N | |
| ATOM | 72 | CA | GLN | A | 11 | 111.508 | 28.195 | 3.353 | 1.00 | 19.09 | C | |
| ATOM | 73 | C | GLN | A | 11 | 111.826 | 29.669 | 3.683 | 1.00 | 20.86 | C | |
| ATOM | 74 | O | GLN | A | 11 | 112.908 | 29.984 | 4.141 | 1.00 | 19.21 | O | |
| ATOM | 75 | CB | GLN | A | 11 | 111.741 | 27.941 | 1.867 | 1.00 | 19.48 | C | |
| ATOM | 76 | CG | GLN | A | 11 | 113.193 | 28.151 | 1.455 | 1.00 | 17.84 | C | |
| ATOM | 77 | CD | GLN | A | 11 | 114.118 | 27.096 | 2.018 | 1.00 | 21.25 | C | |
| ATOM | 78 | OE1 | GLN | A | 11 | 113.948 | 25.909 | 1.753 | 1.00 | 22.82 | O | |
| ATOM | 79 | NE2 | GLN | A | 11 | 115.128 | 27.530 | 2.800 | 1.00 | 21.51 | N | |
| ATOM | 80 | N | LYS | A | 12 | 110.854 | 30.559 | 3.470 | 1.00 | 22.63 | N | |
| ATOM | 81 | CA | LYS | A | 12 | 111.066 | 31.994 | 3.811 | 1.00 | 22.40 | C | |
| ATOM | 82 | C | LYS | A | 12 | 111.381 | 32.168 | 5.312 | 1.00 | 20.21 | C | |
| ATOM | 83 | O | LYS | A | 12 | 112.272 | 32.910 | 5.698 | 1.00 | 23.98 | O | |
| ATOM | 84 | CB | LYS | A | 12 | 109.822 | 32.814 | 3.459 | 1.00 | 25.65 | C | |
| ATOM | 85 | CG | LYS | A | 12 | 109.973 | 34.323 | 3.790 | 1.00 | 27.19 | C | |
| ATOM | 86 | CD | LYS | A | 12 | 108.695 | 35.125 | 3.462 | 1.00 | 31.07 | C | |
| ATOM | 87 | CE | LYS | A | 12 | 108.955 | 36.634 | 3.501 | 1.00 | 34.80 | C | |
| ATOM | 88 | NZ | LYS | A | 12 | 109.330 | 37.061 | 4.866 | 1.00 | 38.91 | N | |
| ATOM | 89 | N | GLY | A | 13 | 110.655 | 31.468 | 6.171 | 1.00 | 18.40 | N | |
| ATOM | 90 | CA | GLY | A | 13 | 110.922 | 31.561 | 7.587 | 1.00 | 16.33 | C | |
| ATOM | 91 | C | GLY | A | 13 | 112.261 | 30.955 | 7.970 | 1.00 | 17.16 | C | |
| ATOM | 92 | O | GLY | A | 13 | 112.974 | 31.471 | 8.839 | 1.00 | 17.11 | O | |
| ATOM | 93 | N | LEU | A | 14 | 112.610 | 29.836 | 7.337 | 1.00 | 18.18 | N | |
| ATOM | 94 | CA | LEU | A | 14 | 113.891 | 29.232 | 7.632 | 1.00 | 19.55 | C | |
| ATOM | 95 | C | LEU | A | 14 | 114.998 | 30.128 | 7.069 | 1.00 | 21.19 | C | |
| ATOM | 96 | O | LEU | A | 14 | 116.057 | 30.158 | 7.650 | 1.00 | 24.39 | O | |
| ATOM | 97 | CB | LEU | A | 14 | 113.972 | 27.827 | 7.044 | 1.00 | 19.38 | C | |
| ATOM | 98 | CG | LEU | A | 14 | 113.038 | 26.842 | 7.771 | 1.00 | 21.89 | C | |
| ATOM | 99 | CD1 | LEU | A | 14 | 113.090 | 25.416 | 7.144 | 1.00 | 23.91 | C | |
| ATOM | 100 | CD2 | LEU | A | 14 | 113.487 | 26.767 | 9.233 | 1.00 | 22.76 | C | |
| ATOM | 101 | N | ASP | A | 15 | 114.751 | 30.853 | 5.961 | 1.00 | 22.66 | N | |
| ATOM | 102 | CA | ASP | A | 15 | 115.777 | 31.781 | 5.370 | 1.00 | 24.92 | C | |
| ATOM | 103 | C | ASP | A | 15 | 116.022 | 32.883 | 6.445 | 1.00 | 25.67 | C | |
| ATOM | 104 | O | ASP | A | 15 | 117.154 | 33.329 | 6.682 | 1.00 | 27.66 | O | |
| ATOM | 105 | CB | ASP | A | 15 | 115.286 | 32.518 | 4.095 | 1.00 | 23.19 | C | |
| ATOM | 106 | CG | ASP | A | 15 | 115.175 | 31.617 | 2.837 | 1.00 | 27.87 | C | |
| ATOM | 107 | OD1 | ASP | A | 15 | 115.755 | 30.513 | 2.774 | 1.00 | 19.50 | O | |
| ATOM | 108 | OD2 | ASP | A | 15 | 114.523 | 32.057 | 1.872 | 1.00 | 28.04 | O | |
| ATOM | 109 | N | TYR | A | 16 | 114.953 | 33.356 | 7.067 | 1.00 | 22.85 | N | |
| ATOM | 110 | CA | TYR | A | 16 | 115.121 | 34.367 | 8.122 | 1.00 | 21.53 | C | |
| ATOM | 111 | C | TYR | A | 16 | 115.809 | 33.782 | 9.370 | 1.00 | 22.53 | C | |
| ATOM | 112 | O | TYR | A | 16 | 116.688 | 34.415 | 10.002 | 1.00 | 22.77 | O | |
| ATOM | 113 | CB | TYR | A | 16 | 113.751 | 34.890 | 8.536 | 1.00 | 19.47 | C | |
| ATOM | 114 | CG | TYR | A | 16 | 113.829 | 36.018 | 9.539 | 1.00 | 22.07 | C | |
| ATOM | 115 | CD1 | TYR | A | 16 | 114.312 | 37.268 | 9.175 | 1.00 | 23.27 | C | |
| ATOM | 116 | CD2 | TYR | A | 16 | 113.407 | 35.828 | 10.843 | 1.00 | 18.37 | C | |
| ATOM | 117 | CE1 | TYR | A | 16 | 114.364 | 38.319 | 10.113 | 1.00 | 24.86 | C | |
| ATOM | 118 | CE2 | TYR | A | 16 | 113.453 | 36.849 | 11.780 | 1.00 | 19.95 | C | |
| ATOM | 119 | CZ | TYR | A | 16 | 113.935 | 38.102 | 11.400 | 1.00 | 22.39 | C | |
| ATOM | 120 | OH | TYR | A | 16 | 114.002 | 39.118 | 12.324 | 1.00 | 23.77 | O | |
| ATOM | 121 | N | ALA | A | 17 | 115.422 | 32.568 | 9.767 | 1.00 | 22.91 | N | |
| ATOM | 122 | CA | ALA | A | 17 | 116.047 | 31.995 | 10.941 | 1.00 | 22.31 | C | |
| ATOM | 123 | C | ALA | A | 17 | 117.536 | 31.815 | 10.688 | 1.00 | 24.09 | C | |
| ATOM | 124 | O | ALA | A | 17 | 118.368 | 32.006 | 11.609 | 1.00 | 26.66 | O | |
| ATOM | 125 | CB | ALA | A | 17 | 115.407 | 30.625 | 11.304 | 1.00 | 23.16 | C | |
| ATOM | 126 | N | SER | A | 18 | 117.874 | 31.460 | 9.448 | 1.00 | 23.70 | N | |
| ATOM | 127 | CA | SER | A | 18 | 119.286 | 31.233 | 9.074 | 1.00 | 27.25 | C | |
| ATOM | 128 | C | SER | A | 18 | 120.097 | 32.533 | 9.307 | 1.00 | 27.23 | C | |
| ATOM | 129 | O | SER | A | 18 | 121.153 | 32.500 | 9.928 | 1.00 | 27.40 | O | |
| ATOM | 130 | CB | SER | A | 18 | 119.370 | 30.763 | 7.604 | 1.00 | 23.88 | C | |
| ATOM | 131 | OG | SER | A | 18 | 120.718 | 30.598 | 7.137 | 1.00 | 27.24 | O | |
| ATOM | 132 | N | GLN | A | 19 | 119.547 | 33.657 | 8.853 | 1.00 | 29.26 | N | |
| ATOM | 133 | CA | GLN | A | 19 | 120.178 | 34.965 | 8.970 | 1.00 | 30.47 | C | |
| ATOM | 134 | C | GLN | A | 19 | 120.407 | 35.319 | 10.441 | 1.00 | 29.93 | C | |
| ATOM | 135 | O | GLN | A | 19 | 121.474 | 35.834 | 10.800 | 1.00 | 27.60 | O | |
| ATOM | 136 | CB | GLN | A | 19 | 119.295 | 35.992 | 8.254 | 1.00 | 34.56 | C | |
| ATOM | 137 | CG | GLN | A | 19 | 119.607 | 37.460 | 8.471 | 1.00 | 43.09 | C | |
| ATOM | 138 | CD | GLN | A | 19 | 118.506 | 38.356 | 7.886 | 1.00 | 48.28 | C | |
| ATOM | 139 | OE1 | GLN | A | 19 | 118.520 | 38.692 | 6.693 | 1.00 | 53.27 | O | |
| ATOM | 140 | NE2 | GLN | A | 19 | 117.541 | 38.718 | 8.714 | 1.00 | 49.14 | N | |
| ATOM | 141 | N | GLN | A | 20 | 119.430 | 35.023 | 11.301 | 1.00 | 27.81 | N | |
| ATOM | 142 | CA | GLN | A | 20 | 119.579 | 35.331 | 12.720 | 1.00 | 26.88 | C | |
| ATOM | 143 | C | GLN | A | 20 | 120.591 | 34.436 | 13.410 | 1.00 | 27.29 | C | |
| ATOM | 144 | O | GLN | A | 20 | 121.374 | 34.902 | 14.246 | 1.00 | 28.18 | O | |
| ATOM | 145 | CB | GLN | A | 20 | 118.224 | 35.233 | 13.459 | 1.00 | 27.06 | C | |
| ATOM | 146 | CG | GLN | A | 20 | 117.201 | 36.127 | 12.863 | 1.00 | 29.89 | C | |
| ATOM | 147 | CD | GLN | A | 20 | 117.695 | 37.542 | 12.736 | 1.00 | 30.73 | C | |
| ATOM | 148 | OE1 | GLN | A | 20 | 117.616 | 38.132 | 11.671 | 1.00 | 42.34 | O | |
| ATOM | 149 | NE2 | GLN | A | 20 | 118.224 | 38.087 | 13.816 | 1.00 | 27.79 | N | |
| ATOM | 150 | N | GLY | A | 21 | 120.579 | 33.143 | 13.094 | 1.00 | 26.15 | N | |
| ATOM | 151 | CA | GLY | A | 21 | 121.532 | 32.247 | 13.744 | 1.00 | 26.98 | C | |
| ATOM | 152 | C | GLY | A | 21 | 122.952 | 32.535 | 13.251 | 1.00 | 27.76 | C | |
| ATOM | 153 | O | GLY | A | 21 | 123.943 | 32.364 | 13.976 | 1.00 | 29.86 | O | |
| ATOM | 154 | N | THR | A | 22 | 123.056 | 32.980 | 12.007 | 1.00 | 27.86 | N | |
| ATOM | 155 | CA | THR | A | 22 | 124.376 | 33.293 | 11.453 | 1.00 | 29.40 | C | |
| ATOM | 156 | C | THR | A | 22 | 124.942 | 34.519 | 12.183 | 1.00 | 30.38 | C | |
| ATOM | 157 | O | THR | A | 22 | 126.147 | 34.587 | 12.470 | 1.00 | 31.00 | O | |
| ATOM | 158 | CB | THR | A | 22 | 124.285 | 33.591 | 9.960 | 1.00 | 28.72 | C | |
| ATOM | 159 | OG1 | THR | A | 22 | 123.980 | 32.365 | 9.253 | 1.00 | 27.14 | O | |
| ATOM | 160 | CG2 | THR | A | 22 | 125.626 | 34.176 | 9.453 | 1.00 | 26.69 | C | |
| ATOM | 161 | N | ALA | A | 23 | 124.070 | 35.474 | 12.493 | 1.00 | 32.27 | N | |
| ATOM | 162 | CA | ALA | A | 23 | 124.522 | 36.681 | 13.205 | 1.00 | 31.91 | C | |
| ATOM | 163 | C | ALA | A | 23 | 125.080 | 36.300 | 14.556 | 1.00 | 31.53 | C | |
| ATOM | 164 | O | ALA | A | 23 | 126.148 | 36.793 | 14.947 | 1.00 | 33.72 | O | |
| ATOM | 165 | CB | ALA | A | 23 | 123.388 | 37.685 | 13.371 | 1.00 | 32.44 | C | |
| ATOM | 166 | N | ALA | A | 24 | 124.377 | 35.432 | 15.287 | 1.00 | 30.88 | N | |
| ATOM | 167 | CA | ALA | A | 24 | 124.860 | 34.999 | 16.592 | 1.00 | 31.01 | C | |
| ATOM | 168 | C | ALA | A | 24 | 126.163 | 34.233 | 16.449 | 1.00 | 32.95 | C | |
| ATOM | 169 | O | ALA | A | 24 | 127.132 | 34.487 | 17.167 | 1.00 | 30.51 | O | |
| ATOM | 170 | CB | ALA | A | 24 | 123.838 | 34.129 | 17.283 | 1.00 | 33.04 | C | |
| ATOM | 171 | N | LEU | A | 25 | 126.200 | 33.309 | 15.501 | 1.00 | 30.79 | N | |
| ATOM | 172 | CA | LEU | A | 25 | 127.389 | 32.503 | 15.305 | 1.00 | 32.62 | C | |
| ATOM | 173 | C | LEU | A | 25 | 128.633 | 33.330 | 14.924 | 1.00 | 32.00 | C | |
| ATOM | 174 | O | LEU | A | 25 | 129.758 | 33.012 | 15.331 | 1.00 | 32.68 | O | |
| ATOM | 175 | CB | LEU | A | 25 | 127.110 | 31.453 | 14.229 | 1.00 | 29.16 | C | |
| ATOM | 176 | CG | LEU | A | 25 | 128.255 | 30.501 | 13.907 | 1.00 | 31.29 | C | |
| ATOM | 177 | CD1 | LEU | A | 25 | 128.585 | 29.619 | 15.079 | 1.00 | 25.06 | C | |
| ATOM | 178 | CD2 | LEU | A | 25 | 127.829 | 29.645 | 12.714 | 1.00 | 26.52 | C | |
| ATOM | 179 | N | AGLN | A | 26 | 128.413 | 34.388 | 14.151 | 0.50 | 31.93 | N | |
| ATOM | 180 | N | BGLN | A | 26 | 128.422 | 34.388 | 14.146 | 0.50 | 32.36 | N | |
| ATOM | 181 | CA | AGLN | A | 26 | 129.494 | 35.258 | 13.704 | 0.50 | 32.03 | C | |
| ATOM | 182 | CA | BGLN | A | 26 | 129.520 | 35.239 | 13.701 | 0.50 | 32.83 | C | |
| ATOM | 183 | C | AGLN | A | 26 | 130.257 | 35.902 | 14.864 | 0.50 | 33.14 | C | |
| ATOM | 184 | C | BGLN | A | 26 | 130.266 | 35.892 | 14.872 | 0.50 | 33.63 | C | |
| ATOM | 185 | O | AGLN | A | 26 | 131.483 | 36.046 | 14.811 | 0.50 | 31.31 | O | |
| ATOM | 186 | O | BGLN | A | 26 | 131.493 | 36.027 | 14.835 | 0.50 | 31.80 | O | |
| ATOM | 187 | CB | AGLN | A | 26 | 128.933 | 36.332 | 12.763 | 0.50 | 32.73 | C | |
| ATOM | 188 | CB | BGLN | A | 26 | 128.998 | 36.307 | 12.730 | 0.50 | 34.28 | C | |
| ATOM | 189 | CG | AGLN | A | 26 | 129.734 | 37.619 | 12.705 | 0.50 | 33.34 | C | |
| ATOM | 190 | CG | BGLN | A | 26 | 130.050 | 37.294 | 12.268 | 0.50 | 36.57 | C | |
| ATOM | 191 | CD | AGLN | A | 26 | 129.339 | 38.625 | 13.784 | 0.50 | 33.87 | C | |
| ATOM | 192 | CD | BGLN | A | 26 | 129.516 | 38.330 | 11.297 | 0.50 | 37.41 | C | |
| ATOM | 193 | OE1 | AGLN | A | 26 | 130.178 | 39.374 | 14.271 | 0.50 | 35.74 | O | |
| ATOM | 194 | OE1 | BGLN | A | 26 | 129.289 | 38.048 | 10.122 | 0.50 | 41.63 | O | |
| ATOM | 195 | NE2 | AGLN | A | 26 | 128.054 | 38.657 | 14.145 | 0.50 | 35.30 | N | |
| ATOM | 196 | NE2 | BGLN | A | 26 | 129.307 | 39.543 | 11.790 | 0.50 | 41.87 | N | |
| ATOM | 197 | N | LYS | A | 27 | 129.532 | 36.294 | 15.910 | 1.00 | 32.03 | N | |
| ATOM | 198 | CA | LYS | A | 27 | 130.175 | 36.902 | 17.069 | 1.00 | 33.30 | C | |
| ATOM | 199 | C | LYS | A | 27 | 131.027 | 35.858 | 17.813 | 1.00 | 33.11 | C | |
| ATOM | 200 | O | LYS | A | 27 | 132.120 | 36.164 | 18.292 | 1.00 | 35.00 | O | |
| ATOM | 201 | CB | LYS | A | 27 | 129.130 | 37.492 | 18.021 | 1.00 | 35.38 | C | |
| ATOM | 202 | CG | LYS | A | 27 | 128.325 | 38.631 | 17.408 | 1.00 | 40.86 | C | |
| ATOM | 203 | CD | LYS | A | 27 | 127.531 | 39.326 | 18.496 | 1.00 | 41.98 | C | |
| ATOM | 204 | CE | LYS | A | 27 | 126.563 | 40.325 | 17.904 | 1.00 | 45.37 | C | |
| ATOM | 205 | NZ | LYS | A | 27 | 125.510 | 39.595 | 17.137 | 1.00 | 46.47 | N | |
| ATOM | 206 | N | GLU | A | 28 | 130.536 | 34.629 | 17.903 | 1.00 | 30.90 | N | |
| ATOM | 207 | CA | GLU | A | 28 | 131.260 | 33.553 | 18.580 | 1.00 | 31.44 | C | |
| ATOM | 208 | C | GLU | A | 28 | 132.476 | 33.067 | 17.783 | 1.00 | 30.26 | C | |
| ATOM | 209 | O | GLU | A | 28 | 133.509 | 32.709 | 18.351 | 1.00 | 33.05 | O | |
| ATOM | 210 | CB | GLU | A | 28 | 130.294 | 32.385 | 18.820 | 1.00 | 33.67 | C | |
| ATOM | 211 | CG | GLU | A | 28 | 129.128 | 32.823 | 19.673 | 1.00 | 40.80 | C | |
| ATOM | 212 | CD | GLU | A | 28 | 128.043 | 31.779 | 19.781 | 1.00 | 45.88 | C | |
| ATOM | 213 | OE1 | GLU | A | 28 | 128.393 | 30.620 | 20.068 | 1.00 | 48.70 | O | |
| ATOM | 214 | OE2 | GLU | A | 28 | 126.847 | 32.130 | 19.601 | 1.00 | 48.24 | O | |
| ATOM | 215 | N | LEU | A | 29 | 132.352 | 33.047 | 16.466 | 1.00 | 27.78 | N | |
| ATOM | 216 | CA | LEU | A | 29 | 133.458 | 32.616 | 15.628 | 1.00 | 28.25 | C | |
| ATOM | 217 | C | LEU | A | 29 | 134.595 | 33.617 | 15.735 | 1.00 | 28.09 | C | |
| ATOM | 218 | O | LEU | A | 29 | 135.778 | 33.260 | 15.739 | 1.00 | 26.00 | O | |
| ATOM | 219 | CB | LEU | A | 29 | 132.998 | 32.496 | 14.162 | 1.00 | 26.14 | C | |
| ATOM | 220 | CG | LEU | A | 29 | 132.057 | 31.308 | 13.932 | 1.00 | 25.62 | C | |
| ATOM | 221 | CD1 | LEU | A | 29 | 131.570 | 31.323 | 12.502 | 1.00 | 28.21 | C | |
| ATOM | 222 | CD2 | LEU | A | 29 | 132.788 | 30.010 | 14.282 | 1.00 | 27.72 | C | |
| ATOM | 223 | N | LYS | A | 30 | 134.213 | 34.885 | 15.805 | 1.00 | 30.04 | N | |
| ATOM | 224 | CA | LYS | A | 30 | 135.184 | 35.947 | 15.898 | 1.00 | 33.25 | C | |
| ATOM | 225 | C | LYS | A | 30 | 136.012 | 35.843 | 17.172 | 1.00 | 34.88 | C | |
| ATOM | 226 | O | LYS | A | 30 | 137.089 | 36.454 | 17.253 | 1.00 | 35.29 | O | |
| ATOM | 227 | CB | LYS | A | 30 | 134.457 | 37.288 | 15.833 | 1.00 | 36.23 | C | |
| ATOM | 228 | CG | LYS | A | 30 | 135.348 | 38.478 | 15.553 | 1.00 | 42.33 | C | |
| ATOM | 229 | CD | LYS | A | 30 | 134.492 | 39.737 | 15.405 | 1.00 | 45.91 | C | |
| ATOM | 230 | CE | LYS | A | 30 | 135.274 | 40.886 | 14.814 | 1.00 | 47.06 | C | |
| ATOM | 231 | NZ | LYS | A | 30 | 136.371 | 41.320 | 15.715 | 1.00 | 49.41 | N | |
| ATOM | 232 | N | ARG | A | 31 | 135.535 | 35.066 | 18.151 | 1.00 | 33.63 | N | |
| ATOM | 233 | CA | ARG | A | 31 | 136.250 | 34.888 | 19.425 | 1.00 | 36.10 | C | |
| ATOM | 234 | C | ARG | A | 31 | 137.212 | 33.687 | 19.424 | 1.00 | 34.55 | C | |
| ATOM | 235 | O | ARG | A | 31 | 137.882 | 33.409 | 20.417 | 1.00 | 35.67 | O | |
| ATOM | 236 | CB | ARG | A | 31 | 135.261 | 34.738 | 20.606 | 1.00 | 37.98 | C | |
| ATOM | 237 | CG | ARG | A | 31 | 134.595 | 36.044 | 21.104 | 1.00 | 41.56 | C | |
| ATOM | 238 | CD | ARG | A | 31 | 133.502 | 35.723 | 22.120 | 1.00 | 45.50 | C | |
| ATOM | 239 | NE | ARG | A | 31 | 133.252 | 34.277 | 22.142 | 1.00 | 52.02 | N | |
| ATOM | 240 | CZ | ARG | A | 31 | 132.081 | 33.692 | 22.409 | 1.00 | 53.02 | C | |
| ATOM | 241 | NH1 | ARG | A | 31 | 131.001 | 34.414 | 22.694 | 1.00 | 53.23 | N | |
| ATOM | 242 | NH2 | ARG | A | 31 | 131.984 | 32.370 | 22.355 | 1.00 | 54.81 | N | |
| ATOM | 243 | N | ILE | A | 32 | 137.256 | 32.947 | 18.330 | 1.00 | 31.42 | N | |
| ATOM | 244 | CA | ILE | A | 32 | 138.181 | 31.823 | 18.259 | 1.00 | 28.65 | C | |
| ATOM | 245 | C | ILE | A | 32 | 139.593 | 32.338 | 18.597 | 1.00 | 32.86 | C | |
| ATOM | 246 | O | ILE | A | 32 | 140.020 | 33.391 | 18.125 | 1.00 | 30.92 | O | |
| ATOM | 247 | CB | ILE | A | 32 | 138.138 | 31.209 | 16.841 | 1.00 | 27.46 | C | |
| ATOM | 248 | CG1 | ILE | A | 32 | 136.832 | 30.388 | 16.706 | 1.00 | 26.62 | C | |
| ATOM | 249 | CG2 | ILE | A | 32 | 139.371 | 30.330 | 16.582 | 1.00 | 27.04 | C | |
| ATOM | 250 | CD1 | ILE | A | 32 | 136.461 | 30.010 | 15.260 | 1.00 | 28.50 | C | |
| ATOM | 251 | N | LYS | A | 33 | 140.293 | 31.598 | 19.446 | 1.00 | 31.18 | N | |
| ATOM | 252 | CA | LYS | A | 33 | 141.643 | 31.965 | 19.812 | 1.00 | 32.80 | C | |
| ATOM | 253 | C | LYS | A | 33 | 142.589 | 31.133 | 18.939 | 1.00 | 30.39 | C | |
| ATOM | 254 | O | LYS | A | 33 | 142.700 | 29.900 | 19.087 | 1.00 | 27.22 | O | |
| ATOM | 255 | CB | LYS | A | 33 | 141.876 | 31.688 | 21.299 | 1.00 | 33.91 | C | |
| ATOM | 256 | N | ILE | A | 34 | 143.279 | 31.815 | 18.023 | 1.00 | 29.33 | N | |
| ATOM | 257 | CA | ILE | A | 34 | 144.196 | 31.126 | 17.119 | 1.00 | 28.76 | C | |
| ATOM | 258 | C | ILE | A | 34 | 145.556 | 30.908 | 17.810 | 1.00 | 29.96 | C | |
| ATOM | 259 | O | ILE | A | 34 | 146.093 | 31.843 | 18.415 | 1.00 | 28.31 | O | |
| ATOM | 260 | CB | ILE | A | 34 | 144.451 | 31.946 | 15.889 | 1.00 | 29.43 | C | |
| ATOM | 261 | CG1 | ILE | A | 34 | 143.116 | 32.387 | 15.261 | 1.00 | 34.18 | C | |
| ATOM | 262 | CG2 | ILE | A | 34 | 145.238 | 31.129 | 14.905 | 1.00 | 28.56 | C | |
| ATOM | 263 | CD1 | ILE | A | 34 | 143.279 | 33.057 | 13.907 | 1.00 | 34.83 | C | |
| ATOM | 264 | N | PRO | A | 35 | 146.095 | 29.676 | 17.748 | 1.00 | 25.45 | N | |
| ATOM | 265 | CA | PRO | A | 35 | 147.387 | 29.333 | 18.361 | 1.00 | 24.11 | C | |
| ATOM | 266 | C | PRO | A | 35 | 148.567 | 30.033 | 17.647 | 1.00 | 27.07 | C | |
| ATOM | 267 | O | PRO | A | 35 | 148.472 | 30.384 | 16.469 | 1.00 | 25.07 | O | |
| ATOM | 268 | CB | PRO | A | 35 | 147.532 | 27.825 | 18.104 | 1.00 | 29.74 | C | |
| ATOM | 269 | CG | PRO | A | 35 | 146.169 | 27.338 | 17.732 | 1.00 | 31.75 | C | |
| ATOM | 270 | CD | PRO | A | 35 | 145.495 | 28.518 | 17.065 | 1.00 | 26.30 | C | |
| ATOM | 271 | N | ASP | A | 36 | 149.668 | 30.223 | 18.374 | 1.00 | 25.04 | N | |
| ATOM | 272 | CA | ASP | A | 36 | 150.909 | 30.750 | 17.791 | 1.00 | 24.93 | C | |
| ATOM | 273 | C | ASP | A | 36 | 151.548 | 29.579 | 17.030 | 1.00 | 23.50 | C | |
| ATOM | 274 | O | ASP | A | 36 | 151.391 | 28.420 | 17.438 | 1.00 | 24.29 | O | |
| ATOM | 275 | CB | ASP | A | 36 | 151.913 | 31.131 | 18.890 | 1.00 | 23.16 | C | |
| ATOM | 276 | CG | ASP | A | 36 | 151.524 | 32.344 | 19.647 | 1.00 | 24.72 | C | |
| ATOM | 277 | OD1 | ASP | A | 36 | 150.586 | 33.084 | 19.222 | 1.00 | 23.61 | O | |
| ATOM | 278 | OD2 | ASP | A | 36 | 152.201 | 32.578 | 20.694 | 1.00 | 27.32 | O | |
| ATOM | 279 | N | TYR | A | 37 | 152.281 | 29.871 | 15.951 | 1.00 | 23.21 | N | |
| ATOM | 280 | CA | TYR | A | 37 | 152.984 | 28.847 | 15.167 | 1.00 | 25.87 | C | |
| ATOM | 281 | C | TYR | A | 37 | 154.477 | 29.185 | 15.058 | 1.00 | 28.85 | C | |
| ATOM | 282 | O | TYR | A | 37 | 154.815 | 30.243 | 14.556 | 1.00 | 29.19 | O | |
| ATOM | 283 | CB | TYR | A | 37 | 152.404 | 28.757 | 13.745 | 1.00 | 28.54 | C | |
| ATOM | 284 | CG | TYR | A | 37 | 151.011 | 28.159 | 13.701 | 1.00 | 28.29 | C | |
| ATOM | 285 | CD1 | TYR | A | 37 | 149.888 | 28.937 | 13.968 | 1.00 | 26.93 | C | |
| ATOM | 286 | CD2 | TYR | A | 37 | 150.838 | 26.784 | 13.494 | 1.00 | 29.30 | C | |
| ATOM | 287 | CE1 | TYR | A | 37 | 148.612 | 28.359 | 14.049 | 1.00 | 26.87 | C | |
| ATOM | 288 | CE2 | TYR | A | 37 | 149.563 | 26.206 | 13.571 | 1.00 | 29.25 | C | |
| ATOM | 289 | CZ | TYR | A | 37 | 148.468 | 27.003 | 13.855 | 1.00 | 28.52 | C | |
| ATOM | 290 | OH | TYR | A | 37 | 147.221 | 26.444 | 14.023 | 1.00 | 30.04 | O | |
| ATOM | 291 | N | SER | A | 38 | 155.374 | 28.308 | 15.503 | 1.00 | 25.37 | N | |
| ATOM | 292 | CA | SER | A | 38 | 156.815 | 28.593 | 15.386 | 1.00 | 27.72 | C | |
| ATOM | 293 | C | SER | A | 38 | 157.466 | 27.438 | 14.673 | 1.00 | 28.75 | C | |
| ATOM | 294 | O | SER | A | 38 | 156.922 | 26.330 | 14.688 | 1.00 | 27.50 | O | |
| ATOM | 295 | CB | SER | A | 38 | 157.495 | 28.766 | 16.761 | 1.00 | 28.15 | C | |
| ATOM | 296 | OG | SER | A | 38 | 157.052 | 29.912 | 17.475 | 1.00 | 31.37 | O | |
| ATOM | 297 | N | ASP | A | 39 | 158.606 | 27.688 | 14.016 | 1.00 | 28.33 | N | |
| ATOM | 298 | CA | ASP | A | 39 | 159.341 | 26.646 | 13.302 | 1.00 | 28.25 | C | |
| ATOM | 299 | C | ASP | A | 39 | 160.769 | 27.083 | 13.017 | 1.00 | 28.61 | C | |
| ATOM | 300 | O | ASP | A | 39 | 161.186 | 28.195 | 13.386 | 1.00 | 29.37 | O | |
| ATOM | 301 | CB | ASP | A | 39 | 158.635 | 26.276 | 11.973 | 1.00 | 28.54 | C | |
| ATOM | 302 | CG | ASP | A | 39 | 158.755 | 24.781 | 11.625 | 1.00 | 33.76 | C | |
| ATOM | 303 | OD1 | ASP | A | 39 | 159.810 | 24.177 | 11.850 | 1.00 | 27.92 | O | |
| ATOM | 304 | OD2 | ASP | A | 39 | 157.787 | 24.193 | 11.085 | 1.00 | 38.71 | O | |
| ATOM | 305 | N | SER | A | 40 | 161.526 | 26.209 | 12.364 | 1.00 | 29.41 | N | |
| ATOM | 306 | CA | SER | A | 40 | 162.921 | 26.499 | 12.020 | 1.00 | 29.70 | C | |
| ATOM | 307 | C | SER | A | 40 | 163.157 | 26.372 | 10.515 | 1.00 | 28.44 | C | |
| ATOM | 308 | O | SER | A | 40 | 162.338 | 25.793 | 9.799 | 1.00 | 29.16 | O | |
| ATOM | 309 | CB | SER | A | 40 | 163.856 | 25.517 | 12.735 | 1.00 | 28.29 | C | |
| ATOM | 310 | OG | SER | A | 40 | 163.803 | 24.250 | 12.054 | 1.00 | 33.28 | O | |
| ATOM | 311 | N | PHE | A | 41 | 164.286 | 26.896 | 10.036 | 1.00 | 26.94 | N | |
| ATOM | 312 | CA | PHE | A | 41 | 164.644 | 26.814 | 8.630 | 1.00 | 33.23 | C | |
| ATOM | 313 | C | PHE | A | 41 | 166.145 | 26.756 | 8.400 | 1.00 | 36.88 | C | |
| ATOM | 314 | O | PHE | A | 41 | 166.933 | 26.980 | 9.309 | 1.00 | 34.29 | O | |
| ATOM | 315 | CB | PHE | A | 41 | 164.094 | 28.042 | 7.866 | 1.00 | 31.34 | C | |
| ATOM | 316 | CG | PHE | A | 41 | 164.715 | 29.359 | 8.283 | 1.00 | 23.47 | C | |
| ATOM | 317 | CD1 | PHE | A | 41 | 165.849 | 29.850 | 7.649 | 1.00 | 27.66 | C | |
| ATOM | 318 | CD2 | PHE | A | 41 | 164.142 | 30.130 | 9.276 | 1.00 | 25.02 | C | |
| ATOM | 319 | CE1 | PHE | A | 41 | 166.392 | 31.096 | 8.007 | 1.00 | 26.86 | C | |
| ATOM | 320 | CE2 | PHE | A | 41 | 164.677 | 31.353 | 9.626 | 1.00 | 26.44 | C | |
| ATOM | 321 | CZ | PHE | A | 41 | 165.812 | 31.836 | 8.981 | 1.00 | 26.39 | C | |
| ATOM | 322 | N | LYS | A | 42 | 166.486 | 26.494 | 7.137 | 1.00 | 42.47 | N | |
| ATOM | 323 | CA | LYS | A | 42 | 167.843 | 26.440 | 6.577 | 1.00 | 47.56 | C | |
| ATOM | 324 | C | LYS | A | 42 | 167.475 | 27.021 | 5.209 | 1.00 | 50.97 | C | |
| ATOM | 325 | O | LYS | A | 42 | 167.348 | 26.289 | 4.220 | 1.00 | 52.20 | O | |
| ATOM | 326 | CB | LYS | A | 42 | 168.309 | 24.991 | 6.415 | 1.00 | 49.73 | C | |
| ATOM | 327 | CG | LYS | A | 42 | 167.821 | 24.075 | 7.522 | 1.00 | 53.87 | C | |
| ATOM | 328 | CD | LYS | A | 42 | 168.219 | 22.619 | 7.278 | 1.00 | 54.49 | C | |
| ATOM | 329 | CE | LYS | A | 42 | 167.454 | 21.705 | 8.234 | 1.00 | 55.43 | C | |
| ATOM | 330 | NZ | LYS | A | 42 | 167.510 | 22.210 | 9.644 | 1.00 | 54.91 | N | |
| ATOM | 331 | N | ILE | A | 43 | 167.231 | 28.331 | 5.175 | 1.00 | 54.51 | N | |
| ATOM | 332 | CA | ILE | A | 43 | 166.802 | 29.038 | 3.954 | 1.00 | 57.47 | C | |
| ATOM | 333 | C | ILE | A | 43 | 166.818 | 30.544 | 4.277 | 1.00 | 60.72 | C | |
| ATOM | 334 | O | ILE | A | 43 | 165.919 | 31.108 | 4.912 | 1.00 | 63.60 | O | |
| ATOM | 335 | CB | ILE | A | 43 | 165.381 | 28.468 | 3.440 | 1.00 | 55.16 | C | |
| ATOM | 336 | CG1 | ILE | A | 43 | 164.635 | 29.468 | 2.545 | 1.00 | 55.28 | C | |
| ATOM | 337 | CG2 | ILE | A | 43 | 164.520 | 28.037 | 4.594 | 1.00 | 57.28 | C | |
| ATOM | 338 | CD1 | ILE | A | 43 | 164.048 | 30.666 | 3.220 | 1.00 | 50.49 | C | |
| ATOM | 339 | N | LYS | A | 44 | 167.916 | 31.150 | 3.846 | 1.00 | 62.14 | N | |
| ATOM | 340 | CA | LYS | A | 44 | 168.292 | 32.548 | 4.025 | 1.00 | 62.63 | C | |
| ATOM | 341 | C | LYS | A | 44 | 167.409 | 33.771 | 4.349 | 1.00 | 62.83 | C | |
| ATOM | 342 | O | LYS | A | 44 | 166.191 | 33.755 | 4.459 | 1.00 | 61.93 | O | |
| ATOM | 343 | CB | LYS | A | 44 | 169.240 | 32.930 | 2.862 | 1.00 | 63.36 | C | |
| ATOM | 344 | N | HIS | A | 45 | 168.186 | 34.838 | 4.453 | 1.00 | 64.30 | N | |
| ATOM | 345 | CA | HIS | A | 45 | 167.944 | 36.229 | 4.800 | 1.00 | 65.63 | C | |
| ATOM | 346 | C | HIS | A | 45 | 169.347 | 36.141 | 5.414 | 1.00 | 66.88 | C | |
| ATOM | 347 | O | HIS | A | 45 | 169.977 | 37.122 | 5.836 | 1.00 | 68.54 | O | |
| ATOM | 348 | CB | HIS | A | 45 | 166.903 | 36.357 | 5.896 | 1.00 | 66.53 | C | |
| ATOM | 349 | N | LEU | A | 46 | 169.764 | 34.864 | 5.420 | 1.00 | 67.18 | N | |
| ATOM | 350 | CA | LEU | A | 46 | 171.008 | 34.268 | 5.892 | 1.00 | 65.88 | C | |
| ATOM | 351 | C | LEU | A | 46 | 170.667 | 32.906 | 6.558 | 1.00 | 65.40 | C | |
| ATOM | 352 | O | LEU | A | 46 | 170.094 | 32.857 | 7.653 | 1.00 | 66.14 | O | |
| ATOM | 353 | CB | LEU | A | 46 | 171.736 | 35.211 | 6.853 | 1.00 | 66.15 | C | |
| ATOM | 354 | CG | LEU | A | 46 | 173.024 | 35.816 | 6.270 | 1.00 | 64.25 | C | |
| ATOM | 355 | CD1 | LEU | A | 46 | 173.954 | 34.688 | 5.847 | 1.00 | 63.74 | C | |
| ATOM | 356 | CD2 | LEU | A | 46 | 172.707 | 36.699 | 5.076 | 1.00 | 64.33 | C | |
| ATOM | 357 | N | GLY | A | 47 | 171.001 | 31.820 | 5.850 | 1.00 | 64.85 | N | |
| ATOM | 358 | CA | GLY | A | 47 | 170.779 | 30.421 | 6.260 | 1.00 | 62.94 | C | |
| ATOM | 359 | C | GLY | A | 47 | 170.024 | 29.849 | 7.476 | 1.00 | 61.36 | C | |
| ATOM | 360 | O | GLY | A | 47 | 168.941 | 29.279 | 7.335 | 1.00 | 62.05 | O | |
| ATOM | 361 | N | LYS | A | 48 | 170.657 | 29.963 | 8.642 | 1.00 | 59.03 | N | |
| ATOM | 362 | CA | LYS | A | 48 | 170.269 | 29.507 | 10.001 | 1.00 | 56.52 | C | |
| ATOM | 363 | C | LYS | A | 48 | 168.870 | 29.261 | 10.669 | 1.00 | 53.42 | C | |
| ATOM | 364 | O | LYS | A | 48 | 168.304 | 28.175 | 10.725 | 1.00 | 54.47 | O | |
| ATOM | 365 | CB | LYS | A | 48 | 171.007 | 30.417 | 10.981 | 1.00 | 58.58 | C | |
| ATOM | 366 | CG | LYS | A | 48 | 172.128 | 31.221 | 10.367 | 1.00 | 58.94 | C | |
| ATOM | 367 | CD | LYS | A | 48 | 173.453 | 30.579 | 10.687 | 1.00 | 58.04 | C | |
| ATOM | 368 | CE | LYS | A | 48 | 174.511 | 31.142 | 9.785 | 1.00 | 59.93 | C | |
| ATOM | 369 | NZ | LYS | A | 48 | 174.347 | 32.612 | 9.676 | 1.00 | 59.13 | N | |
| ATOM | 370 | N | GLY | A | 49 | 168.451 | 30.328 | 11.324 | 1.00 | 48.55 | N | |
| ATOM | 371 | CA | GLY | A | 49 | 167.242 | 30.449 | 12.104 | 1.00 | 36.57 | C | |
| ATOM | 372 | C | GLY | A | 49 | 165.954 | 29.725 | 12.386 | 1.00 | 30.66 | C | |
| ATOM | 373 | O | GLY | A | 49 | 165.681 | 28.544 | 12.124 | 1.00 | 29.43 | O | |
| ATOM | 374 | N | HIS | A | 50 | 165.138 | 30.561 | 13.003 | 1.00 | 28.49 | N | |
| ATOM | 375 | CA | HIS | A | 50 | 163.824 | 30.229 | 13.499 | 1.00 | 24.43 | C | |
| ATOM | 376 | C | HIS | A | 50 | 162.902 | 31.383 | 13.182 | 1.00 | 25.65 | C | |
| ATOM | 377 | O | HIS | A | 50 | 163.348 | 32.525 | 12.946 | 1.00 | 26.86 | O | |
| ATOM | 378 | CB | HIS | A | 50 | 163.896 | 30.090 | 15.019 | 1.00 | 27.44 | C | |
| ATOM | 379 | CG | HIS | A | 50 | 164.803 | 28.993 | 15.464 | 1.00 | 31.84 | C | |
| ATOM | 380 | ND1 | HIS | A | 50 | 164.354 | 27.709 | 15.701 | 1.00 | 32.14 | N | |
| ATOM | 381 | CD2 | HIS | A | 50 | 166.144 | 28.964 | 15.630 | 1.00 | 33.93 | C | |
| ATOM | 382 | CE1 | HIS | A | 50 | 165.384 | 26.932 | 15.984 | 1.00 | 33.40 | C | |
| ATOM | 383 | NE2 | HIS | A | 50 | 166.481 | 27.663 | 15.949 | 1.00 | 36.36 | N | |
| ATOM | 384 | N | TYR | A | 51 | 161.611 | 31.104 | 13.226 | 1.00 | 21.98 | N | |
| ATOM | 385 | CA | TYR | A | 51 | 160.664 | 32.159 | 12.964 | 1.00 | 24.95 | C | |
| ATOM | 386 | C | TYR | A | 51 | 159.329 | 31.830 | 13.662 | 1.00 | 22.65 | C | |
| ATOM | 387 | O | TYR | A | 51 | 159.119 | 30.721 | 14.118 | 1.00 | 23.78 | O | |
| ATOM | 388 | CB | TYR | A | 51 | 160.515 | 32.307 | 11.437 | 1.00 | 24.56 | C | |
| ATOM | 389 | CG | TYR | A | 51 | 159.955 | 31.082 | 10.742 | 1.00 | 22.30 | C | |
| ATOM | 390 | CD1 | TYR | A | 51 | 158.591 | 30.874 | 10.649 | 1.00 | 25.71 | C | |
| ATOM | 391 | CD2 | TYR | A | 51 | 160.795 | 30.134 | 10.207 | 1.00 | 26.44 | C | |
| ATOM | 392 | CE1 | TYR | A | 51 | 158.076 | 29.731 | 10.025 | 1.00 | 24.67 | C | |
| ATOM | 393 | CE2 | TYR | A | 51 | 160.308 | 28.986 | 9.578 | 1.00 | 30.15 | C | |
| ATOM | 394 | CZ | TYR | A | 51 | 158.941 | 28.799 | 9.490 | 1.00 | 25.99 | C | |
| ATOM | 395 | OH | TYR | A | 51 | 158.477 | 27.694 | 8.798 | 1.00 | 33.14 | O | |
| ATOM | 396 | N | SER | A | 52 | 158.447 | 32.818 | 13.787 | 1.00 | 23.46 | N | |
| ATOM | 397 | CA | SER | A | 52 | 157.161 | 32.544 | 14.412 | 1.00 | 24.05 | C | |
| ATOM | 398 | C | SER | A | 52 | 156.082 | 33.505 | 13.944 | 1.00 | 26.47 | C | |
| ATOM | 399 | O | SER | A | 52 | 156.381 | 34.646 | 13.662 | 1.00 | 24.23 | O | |
| ATOM | 400 | CB | SER | A | 52 | 157.338 | 32.633 | 15.955 | 1.00 | 28.28 | C | |
| ATOM | 401 | OG | SER | A | 52 | 156.097 | 32.714 | 16.669 | 1.00 | 30.04 | O | |
| ATOM | 402 | N | PHE | A | 53 | 154.836 | 33.020 | 13.847 | 1.00 | 24.70 | N | |
| ATOM | 403 | CA | PHE | A | 53 | 153.675 | 33.867 | 13.494 | 1.00 | 22.44 | C | |
| ATOM | 404 | C | PHE | A | 53 | 152.849 | 33.792 | 14.790 | 1.00 | 22.83 | C | |
| ATOM | 405 | O | PHE | A | 53 | 152.386 | 32.701 | 15.165 | 1.00 | 26.89 | O | |
| ATOM | 406 | CB | PHE | A | 53 | 152.933 | 33.265 | 12.292 | 1.00 | 21.75 | C | |
| ATOM | 407 | CG | PHE | A | 53 | 153.620 | 33.544 | 10.974 | 1.00 | 20.86 | C | |
| ATOM | 408 | CD1 | PHE | A | 53 | 153.356 | 34.717 | 10.291 | 1.00 | 23.76 | C | |
| ATOM | 409 | CD2 | PHE | A | 53 | 154.613 | 32.695 | 10.493 | 1.00 | 23.46 | C | |
| ATOM | 410 | CE1 | PHE | A | 53 | 154.089 | 35.051 | 9.146 | 1.00 | 23.51 | C | |
| ATOM | 411 | CE2 | PHE | A | 53 | 155.354 | 33.030 | 9.331 | 1.00 | 22.85 | C | |
| ATOM | 412 | CZ | PHE | A | 53 | 155.075 | 34.214 | 8.682 | 1.00 | 19.65 | C | |
| ATOM | 413 | N | TYR | A | 54 | 152.675 | 34.917 | 15.480 | 1.00 | 23.21 | N | |
| ATOM | 414 | CA | TYR | A | 54 | 152.010 | 34.864 | 16.772 | 1.00 | 23.10 | C | |
| ATOM | 415 | C | TYR | A | 54 | 151.084 | 36.005 | 17.155 | 1.00 | 23.18 | C | |
| ATOM | 416 | O | TYR | A | 54 | 150.928 | 36.985 | 16.403 | 1.00 | 23.21 | O | |
| ATOM | 417 | CB | TYR | A | 54 | 153.100 | 34.662 | 17.844 | 1.00 | 24.05 | C | |
| ATOM | 418 | CG | TYR | A | 54 | 154.006 | 35.848 | 17.969 | 1.00 | 25.74 | C | |
| ATOM | 419 | CD1 | TYR | A | 54 | 155.034 | 36.058 | 17.052 | 1.00 | 27.20 | C | |
| ATOM | 420 | CD2 | TYR | A | 54 | 153.803 | 36.800 | 18.986 | 1.00 | 21.85 | C | |
| ATOM | 421 | CE1 | TYR | A | 54 | 155.851 | 37.195 | 17.144 | 1.00 | 29.61 | C | |
| ATOM | 422 | CE2 | TYR | A | 54 | 154.597 | 37.912 | 19.095 | 1.00 | 27.15 | C | |
| ATOM | 423 | CZ | TYR | A | 54 | 155.625 | 38.112 | 18.172 | 1.00 | 30.11 | C | |
| ATOM | 424 | OH | TYR | A | 54 | 156.407 | 39.238 | 18.289 | 1.00 | 33.52 | O | |
| ATOM | 425 | N | SER | A | 55 | 150.454 | 35.891 | 18.330 | 1.00 | 23.77 | N | |
| ATOM | 426 | CA | SER | A | 55 | 149.468 | 36.907 | 18.817 | 1.00 | 22.90 | C | |
| ATOM | 427 | C | SER | A | 55 | 148.474 | 37.298 | 17.734 | 1.00 | 25.18 | C | |
| ATOM | 428 | O | SER | A | 55 | 148.253 | 38.504 | 17.479 | 1.00 | 23.41 | O | |
| ATOM | 429 | CB | SER | A | 55 | 150.143 | 38.195 | 19.324 | 1.00 | 27.38 | C | |
| ATOM | 430 | OG | SER | A | 55 | 150.840 | 37.954 | 20.536 | 1.00 | 34.52 | O | |
| ATOM | 431 | N | MET | A | 56 | 147.897 | 36.289 | 17.094 | 1.00 | 24.74 | N | |
| ATOM | 432 | CA | MET | A | 56 | 146.935 | 36.530 | 16.020 | 1.00 | 25.71 | C | |
| ATOM | 433 | C | MET | A | 56 | 145.557 | 36.808 | 16.615 | 1.00 | 26.78 | C | |
| ATOM | 434 | O | MET | A | 56 | 145.189 | 36.244 | 17.641 | 1.00 | 25.51 | O | |
| ATOM | 435 | CB | MET | A | 56 | 146.857 | 35.307 | 15.112 | 1.00 | 25.53 | C | |
| ATOM | 436 | CG | MET | A | 56 | 147.958 | 35.214 | 14.041 | 1.00 | 23.48 | C | |
| ATOM | 437 | SD | MET | A | 56 | 147.807 | 33.712 | 13.015 | 1.00 | 23.41 | S | |
| ATOM | 438 | CE | MET | A | 56 | 148.853 | 32.542 | 14.022 | 1.00 | 21.48 | C | |
| ATOM | 439 | N | AASP | A | 57 | 144.808 | 37.676 | 15.945 | 0.50 | 25.73 | N | |
| ATOM | 440 | N | BASP | A | 57 | 144.804 | 37.679 | 15.955 | 0.50 | 26.36 | N | |
| ATOM | 441 | CA | AASP | A | 57 | 143.468 | 38.055 | 16.374 | 0.50 | 28.11 | C | |
| ATOM | 442 | CA | BASP | A | 57 | 143.458 | 38.041 | 16.386 | 0.50 | 28.97 | C | |
| ATOM | 443 | C | AASP | A | 57 | 142.578 | 38.134 | 15.136 | 0.50 | 25.78 | C | |
| ATOM | 444 | C | BASP | A | 57 | 142.578 | 38.129 | 15.142 | 0.50 | 26.58 | C | |
| ATOM | 445 | O | AASP | A | 57 | 143.010 | 38.604 | 14.082 | 0.50 | 24.22 | O | |
| ATOM | 446 | O | BASP | A | 57 | 143.016 | 38.603 | 14.093 | 0.50 | 25.12 | O | |
| ATOM | 447 | CB | AASP | A | 57 | 143.504 | 39.431 | 17.053 | 0.50 | 29.83 | C | |
| ATOM | 448 | CB | BASP | A | 57 | 143.460 | 39.407 | 17.087 | 0.50 | 31.89 | C | |
| ATOM | 449 | CG | AASP | A | 57 | 142.140 | 39.864 | 17.563 | 0.50 | 33.07 | C | |
| ATOM | 450 | CG | BASP | A | 57 | 144.059 | 39.355 | 18.480 | 0.50 | 35.05 | C | |
| ATOM | 451 | OD1 | AASP | A | 57 | 141.546 | 39.108 | 18.358 | 0.50 | 36.26 | O | |
| ATOM | 452 | OD1 | BASP | A | 57 | 144.052 | 40.397 | 19.173 | 0.50 | 39.95 | O | |
| ATOM | 453 | OD2 | AASP | A | 57 | 141.660 | 40.953 | 17.172 | 0.50 | 35.15 | O | |
| ATOM | 454 | OD2 | BASP | A | 57 | 144.534 | 38.276 | 18.884 | 0.50 | 39.02 | O | |
| ATOM | 455 | N | ILE | A | 58 | 141.341 | 37.656 | 15.252 | 1.00 | 27.15 | N | |
| ATOM | 456 | CA | ILE | A | 58 | 140.423 | 37.741 | 14.132 | 1.00 | 25.72 | C | |
| ATOM | 457 | C | ILE | A | 58 | 139.821 | 39.168 | 14.128 | 1.00 | 27.58 | C | |
| ATOM | 458 | O | ILE | A | 58 | 139.111 | 39.562 | 15.061 | 1.00 | 30.85 | O | |
| ATOM | 459 | CB | ILE | A | 58 | 139.311 | 36.670 | 14.239 | 1.00 | 25.97 | C | |
| ATOM | 460 | CG1 | ILE | A | 58 | 139.938 | 35.282 | 14.125 | 1.00 | 27.27 | C | |
| ATOM | 461 | CG2 | ILE | A | 58 | 138.288 | 36.868 | 13.108 | 1.00 | 26.50 | C | |
| ATOM | 462 | CD1 | ILE | A | 58 | 139.006 | 34.120 | 14.499 | 1.00 | 28.80 | C | |
| ATOM | 463 | N | ARG | A | 59 | 140.136 | 39.938 | 13.099 | 1.00 | 29.15 | N | |
| ATOM | 464 | CA | ARG | A | 59 | 139.638 | 41.307 | 12.949 | 1.00 | 30.60 | C | |
| ATOM | 465 | C | ARG | A | 59 | 138.252 | 41.275 | 12.315 | 1.00 | 32.65 | C | |
| ATOM | 466 | O | ARG | A | 59 | 137.389 | 42.083 | 12.669 | 1.00 | 32.68 | O | |
| ATOM | 467 | CB | ARG | A | 59 | 140.551 | 42.115 | 12.043 | 1.00 | 36.63 | C | |
| ATOM | 468 | CG | ARG | A | 59 | 141.889 | 42.401 | 12.635 | 1.00 | 41.88 | C | |
| ATOM | 469 | CD | ARG | A | 59 | 141.725 | 43.265 | 13.860 | 1.00 | 49.34 | C | |
| ATOM | 470 | NE | ARG | A | 59 | 142.995 | 43.857 | 14.261 | 1.00 | 56.51 | N | |
| ATOM | 471 | CZ | ARG | A | 59 | 143.729 | 43.429 | 15.283 | 1.00 | 60.31 | C | |
| ATOM | 472 | NH1 | ARG | A | 59 | 143.312 | 42.399 | 16.017 | 1.00 | 60.21 | N | |
| ATOM | 473 | NH2 | ARG | A | 59 | 144.885 | 44.027 | 15.563 | 1.00 | 62.31 | N | |
| ATOM | 474 | N | GLU | A | 60 | 138.055 | 40.356 | 11.370 | 1.00 | 29.22 | N | |
| ATOM | 475 | CA | GLU | A | 60 | 136.761 | 40.190 | 10.695 | 1.00 | 29.61 | C | |
| ATOM | 476 | C | GLU | A | 60 | 136.479 | 38.735 | 10.303 | 1.00 | 29.36 | C | |
| ATOM | 477 | O | GLU | A | 60 | 137.370 | 38.043 | 9.845 | 1.00 | 26.75 | O | |
| ATOM | 478 | CB | GLU | A | 60 | 136.711 | 41.048 | 9.432 | 1.00 | 33.77 | C | |
| ATOM | 479 | CG | GLU | A | 60 | 135.438 | 40.875 | 8.619 | 1.00 | 39.39 | C | |
| ATOM | 480 | CD | GLU | A | 60 | 135.151 | 42.081 | 7.746 | 1.00 | 45.88 | C | |
| ATOM | 481 | OE1 | GLU | A | 60 | 136.062 | 42.507 | 6.977 | 1.00 | 47.87 | O | |
| ATOM | 482 | OE2 | GLU | A | 60 | 134.009 | 42.593 | 7.843 | 1.00 | 50.95 | O | |
| ATOM | 483 | N | PHE | A | 61 | 135.233 | 38.280 | 10.497 | 1.00 | 26.31 | N | |
| ATOM | 484 | CA | PHE | A | 61 | 134.841 | 36.905 | 10.122 | 1.00 | 29.11 | C | |
| ATOM | 485 | C | PHE | A | 61 | 133.548 | 37.172 | 9.333 | 1.00 | 28.32 | C | |
| ATOM | 486 | O | PHE | A | 61 | 132.473 | 37.330 | 9.924 | 1.00 | 32.28 | O | |
| ATOM | 487 | CB | PHE | A | 61 | 134.581 | 36.035 | 11.361 | 1.00 | 27.73 | C | |
| ATOM | 488 | CG | PHE | A | 61 | 134.805 | 34.540 | 11.131 | 1.00 | 28.95 | C | |
| ATOM | 489 | CD1 | PHE | A | 61 | 134.184 | 33.872 | 10.067 | 1.00 | 27.77 | C | |
| ATOM | 490 | CD2 | PHE | A | 61 | 135.641 | 33.810 | 11.978 | 1.00 | 28.24 | C | |
| ATOM | 491 | CE1 | PHE | A | 61 | 134.400 | 32.490 | 9.848 | 1.00 | 26.16 | C | |
| ATOM | 492 | CE2 | PHE | A | 61 | 135.869 | 32.444 | 11.775 | 1.00 | 26.34 | C | |
| ATOM | 493 | CZ | PHE | A | 61 | 135.241 | 31.784 | 10.705 | 1.00 | 29.37 | C | |
| ATOM | 494 | N | GLN | A | 62 | 133.713 | 37.316 | 8.020 | 1.00 | 29.23 | N | |
| ATOM | 495 | CA | GLN | A | 62 | 132.647 | 37.649 | 7.070 | 1.00 | 30.19 | C | |
| ATOM | 496 | C | GLN | A | 62 | 131.866 | 36.417 | 6.667 | 1.00 | 28.99 | C | |
| ATOM | 497 | O | GLN | A | 62 | 132.435 | 35.466 | 6.136 | 1.00 | 25.59 | O | |
| ATOM | 498 | CB | GLN | A | 62 | 133.218 | 38.255 | 5.776 | 1.00 | 35.10 | C | |
| ATOM | 499 | CG | GLN | A | 62 | 133.669 | 39.711 | 5.814 | 1.00 | 44.10 | C | |
| ATOM | 500 | CD | GLN | A | 62 | 133.880 | 40.289 | 4.398 | 1.00 | 47.75 | C | |
| ATOM | 501 | OE1 | GLN | A | 62 | 133.506 | 39.668 | 3.394 | 1.00 | 51.99 | O | |
| ATOM | 502 | NE2 | GLN | A | 62 | 134.470 | 41.479 | 4.322 | 1.00 | 51.59 | N | |
| ATOM | 503 | N | LEU | A | 63 | 130.560 | 36.459 | 6.883 | 1.00 | 33.41 | N | |
| ATOM | 504 | CA | LEU | A | 63 | 129.673 | 35.349 | 6.524 | 1.00 | 32.25 | C | |
| ATOM | 505 | C | LEU | A | 63 | 128.623 | 36.031 | 5.683 | 1.00 | 37.17 | C | |
| ATOM | 506 | O | LEU | A | 63 | 127.551 | 36.355 | 6.190 | 1.00 | 38.61 | O | |
| ATOM | 507 | CB | LEU | A | 63 | 129.043 | 34.775 | 7.771 | 1.00 | 30.33 | C | |
| ATOM | 508 | CG | LEU | A | 63 | 129.994 | 34.266 | 8.867 | 1.00 | 27.52 | C | |
| ATOM | 509 | CD1 | LEU | A | 63 | 129.189 | 33.798 | 10.041 | 1.00 | 31.03 | C | |
| ATOM | 510 | CD2 | LEU | A | 63 | 130.870 | 33.146 | 8.306 | 1.00 | 27.28 | C | |
| ATOM | 511 | N | PRO | A | 64 | 128.928 | 36.272 | 4.399 | 1.00 | 39.32 | N | |
| ATOM | 512 | CA | PRO | A | 64 | 128.160 | 36.927 | 3.328 | 1.00 | 42.91 | C | |
| ATOM | 513 | C | PRO | A | 64 | 126.774 | 36.372 | 3.075 | 1.00 | 44.03 | C | |
| ATOM | 514 | O | PRO | A | 64 | 125.818 | 37.125 | 2.829 | 1.00 | 44.61 | O | |
| ATOM | 515 | CB | PRO | A | 64 | 129.063 | 36.768 | 2.099 | 1.00 | 43.52 | C | |
| ATOM | 516 | CG | PRO | A | 64 | 130.455 | 36.488 | 2.711 | 1.00 | 41.88 | C | |
| ATOM | 517 | CD | PRO | A | 64 | 130.084 | 35.572 | 3.817 | 1.00 | 41.59 | C | |
| ATOM | 518 | N | SER | A | 65 | 126.677 | 35.048 | 3.090 | 1.00 | 42.25 | N | |
| ATOM | 519 | CA | SER | A | 65 | 125.398 | 34.404 | 2.882 | 1.00 | 40.81 | C | |
| ATOM | 520 | C | SER | A | 65 | 125.436 | 33.048 | 3.563 | 1.00 | 39.28 | C | |
| ATOM | 521 | O | SER | A | 65 | 126.507 | 32.458 | 3.788 | 1.00 | 38.70 | O | |
| ATOM | 522 | CB | SER | A | 65 | 125.082 | 34.270 | 1.381 | 1.00 | 41.13 | C | |
| ATOM | 523 | OG | SER | A | 65 | 126.054 | 33.496 | 0.700 | 1.00 | 45.14 | O | |
| ATOM | 524 | N | SER | A | 66 | 124.251 | 32.579 | 3.922 | 1.00 | 36.47 | N | |
| ATOM | 525 | CA | SER | A | 66 | 124.095 | 31.310 | 4.599 | 1.00 | 32.25 | C | |
| ATOM | 526 | C | SER | A | 66 | 122.755 | 30.807 | 4.118 | 1.00 | 27.93 | C | |
| ATOM | 527 | O | SER | A | 66 | 121.990 | 31.570 | 3.533 | 1.00 | 27.87 | O | |
| ATOM | 528 | CB | SER | A | 66 | 124.047 | 31.507 | 6.130 | 1.00 | 34.07 | C | |
| ATOM | 529 | OG | SER | A | 66 | 122.845 | 32.149 | 6.568 | 1.00 | 36.56 | O | |
| ATOM | 530 | N | GLN | A | 67 | 122.474 | 29.536 | 4.347 | 1.00 | 22.30 | N | |
| ATOM | 531 | CA | GLN | A | 67 | 121.184 | 28.985 | 3.936 | 1.00 | 22.45 | C | |
| ATOM | 532 | C | GLN | A | 67 | 120.797 | 27.881 | 4.881 | 1.00 | 23.86 | C | |
| ATOM | 533 | O | GLN | A | 67 | 121.664 | 27.294 | 5.506 | 1.00 | 22.05 | O | |
| ATOM | 534 | CB | GLN | A | 67 | 121.292 | 28.344 | 2.543 | 1.00 | 24.19 | C | |
| ATOM | 535 | CG | GLN | A | 67 | 121.790 | 29.230 | 1.396 | 1.00 | 27.29 | C | |
| ATOM | 536 | CD | GLN | A | 67 | 121.960 | 28.414 | 0.097 | 1.00 | 27.31 | C | |
| ATOM | 537 | OE1 | GLN | A | 67 | 122.800 | 27.511 | 0.030 | 1.00 | 28.49 | O | |
| ATOM | 538 | NE2 | GLN | A | 67 | 121.155 | 28.713 | −0.910 | 1.00 | 25.69 | N | |
| ATOM | 539 | N | ILE | A | 68 | 119.492 | 27.580 | 4.964 | 1.00 | 22.22 | N | |
| ATOM | 540 | CA | ILE | A | 68 | 119.034 | 26.422 | 5.731 | 1.00 | 21.71 | C | |
| ATOM | 541 | C | ILE | A | 68 | 118.310 | 25.603 | 4.644 | 1.00 | 20.86 | C | |
| ATOM | 542 | O | ILE | A | 68 | 117.463 | 26.167 | 3.944 | 1.00 | 26.85 | O | |
| ATOM | 543 | CB | ILE | A | 68 | 118.037 | 26.792 | 6.874 | 1.00 | 23.24 | C | |
| ATOM | 544 | CG1 | ILE | A | 68 | 118.832 | 27.368 | 8.050 | 1.00 | 28.51 | C | |
| ATOM | 545 | CG2 | ILE | A | 68 | 117.242 | 25.526 | 7.301 | 1.00 | 30.03 | C | |
| ATOM | 546 | CD1 | ILE | A | 68 | 119.811 | 26.339 | 8.647 | 1.00 | 33.66 | C | |
| ATOM | 547 | N | SER | A | 69 | 118.688 | 24.331 | 4.462 | 1.00 | 19.48 | N | |
| ATOM | 548 | CA | SER | A | 69 | 118.089 | 23.458 | 3.435 | 1.00 | 19.11 | C | |
| ATOM | 549 | C | SER | A | 69 | 117.155 | 22.488 | 4.140 | 1.00 | 22.11 | C | |
| ATOM | 550 | O | SER | A | 69 | 117.482 | 21.959 | 5.190 | 1.00 | 22.70 | O | |
| ATOM | 551 | CB | SER | A | 69 | 119.156 | 22.601 | 2.719 | 1.00 | 20.87 | C | |
| ATOM | 552 | OG | SER | A | 69 | 120.136 | 23.368 | 2.050 | 1.00 | 29.26 | O | |
| ATOM | 553 | N | MET | A | 70 | 115.988 | 22.249 | 3.560 | 1.00 | 19.12 | N | |
| ATOM | 554 | CA | MET | A | 70 | 115.078 | 21.272 | 4.172 | 1.00 | 19.35 | C | |
| ATOM | 555 | C | MET | A | 70 | 115.367 | 19.925 | 3.501 | 1.00 | 19.79 | C | |
| ATOM | 556 | O | MET | A | 70 | 115.147 | 19.783 | 2.277 | 1.00 | 19.65 | O | |
| ATOM | 557 | CB | MET | A | 70 | 113.628 | 21.673 | 3.931 | 1.00 | 21.18 | C | |
| ATOM | 558 | CG | MET | A | 70 | 113.104 | 22.705 | 4.913 | 1.00 | 22.70 | C | |
| ATOM | 559 | SD | MET | A | 70 | 111.391 | 23.243 | 4.442 | 1.00 | 32.05 | S | |
| ATOM | 560 | CE | MET | A | 70 | 111.903 | 24.536 | 3.733 | 1.00 | 18.63 | C | |
| ATOM | 561 | N | VAL | A | 71 | 115.826 | 18.949 | 4.291 | 1.00 | 17.44 | N | |
| ATOM | 562 | CA | VAL | A | 71 | 116.166 | 17.580 | 3.769 | 1.00 | 18.30 | C | |
| ATOM | 563 | C | VAL | A | 71 | 114.936 | 16.733 | 4.156 | 1.00 | 18.45 | C | |
| ATOM | 564 | O | VAL | A | 71 | 114.738 | 16.375 | 5.318 | 1.00 | 20.54 | O | |
| ATOM | 565 | CB | VAL | A | 71 | 117.435 | 17.022 | 4.451 | 1.00 | 18.82 | C | |
| ATOM | 566 | CG1 | VAL | A | 71 | 117.849 | 15.688 | 3.754 | 1.00 | 20.11 | C | |
| ATOM | 567 | CG2 | VAL | A | 71 | 118.543 | 18.016 | 4.359 | 1.00 | 22.52 | C | |
| ATOM | 568 | N | PRO | A | 72 | 114.111 | 16.359 | 3.168 | 1.00 | 19.94 | N | |
| ATOM | 569 | CA | PRO | A | 72 | 112.908 | 15.617 | 3.522 | 1.00 | 19.94 | C | |
| ATOM | 570 | C | PRO | A | 72 | 113.092 | 14.387 | 4.383 | 1.00 | 22.65 | C | |
| ATOM | 571 | O | PRO | A | 72 | 113.927 | 13.537 | 4.051 | 1.00 | 22.68 | O | |
| ATOM | 572 | CB | PRO | A | 72 | 112.261 | 15.347 | 2.160 | 1.00 | 19.94 | C | |
| ATOM | 573 | CG | PRO | A | 72 | 112.880 | 16.344 | 1.238 | 1.00 | 20.80 | C | |
| ATOM | 574 | CD | PRO | A | 72 | 114.292 | 16.411 | 1.709 | 1.00 | 22.27 | C | |
| ATOM | 575 | N | ASN | A | 73 | 112.321 | 14.333 | 5.482 | 1.00 | 24.03 | N | |
| ATOM | 576 | CA | ASN | A | 73 | 112.298 | 13.230 | 6.435 | 1.00 | 24.79 | C | |
| ATOM | 577 | C | ASN | A | 73 | 113.597 | 12.986 | 7.160 | 1.00 | 25.88 | C | |
| ATOM | 578 | O | ASN | A | 73 | 113.772 | 11.921 | 7.769 | 1.00 | 24.35 | O | |
| ATOM | 579 | CB | ASN | A | 73 | 111.904 | 11.925 | 5.707 | 1.00 | 26.72 | C | |
| ATOM | 580 | CG | ASN | A | 73 | 110.612 | 12.052 | 4.938 | 1.00 | 26.60 | C | |
| ATOM | 581 | OD1 | ASN | A | 73 | 109.578 | 12.391 | 5.486 | 1.00 | 28.50 | O | |
| ATOM | 582 | ND2 | ASN | A | 73 | 110.680 | 11.801 | 3.662 | 1.00 | 29.42 | N | |
| ATOM | 583 | N | VAL | A | 74 | 114.506 | 13.961 | 7.101 | 1.00 | 23.53 | N | |
| ATOM | 584 | CA | VAL | A | 74 | 115.790 | 13.818 | 7.757 | 1.00 | 22.72 | C | |
| ATOM | 585 | C | VAL | A | 74 | 115.994 | 14.966 | 8.748 | 1.00 | 23.25 | C | |
| ATOM | 586 | O | VAL | A | 74 | 116.283 | 14.739 | 9.913 | 1.00 | 21.64 | O | |
| ATOM | 587 | CB | VAL | A | 74 | 116.925 | 13.832 | 6.704 | 1.00 | 23.56 | C | |
| ATOM | 588 | CG1 | VAL | A | 74 | 118.259 | 13.786 | 7.377 | 1.00 | 24.37 | C | |
| ATOM | 589 | CG2 | VAL | A | 74 | 116.764 | 12.657 | 5.735 | 1.00 | 22.92 | C | |
| ATOM | 590 | N | GLY | A | 75 | 115.791 | 16.201 | 8.285 | 1.00 | 18.56 | N | |
| ATOM | 591 | CA | GLY | A | 75 | 116.026 | 17.339 | 9.162 | 1.00 | 20.15 | C | |
| ATOM | 592 | C | GLY | A | 75 | 116.504 | 18.566 | 8.369 | 1.00 | 19.09 | C | |
| ATOM | 593 | O | GLY | A | 75 | 116.192 | 18.714 | 7.194 | 1.00 | 17.07 | O | |
| ATOM | 594 | N | LEU | A | 76 | 117.291 | 19.422 | 9.009 | 1.00 | 18.27 | N | |
| ATOM | 595 | CA | LEU | A | 76 | 117.780 | 20.636 | 8.352 | 1.00 | 16.25 | C | |
| ATOM | 596 | C | LEU | A | 76 | 119.284 | 20.610 | 8.125 | 1.00 | 21.88 | C | |
| ATOM | 597 | O | LEU | A | 76 | 120.027 | 19.940 | 8.870 | 1.00 | 24.75 | O | |
| ATOM | 598 | CB | LEU | A | 76 | 117.427 | 21.855 | 9.196 | 1.00 | 19.42 | C | |
| ATOM | 599 | CG | LEU | A | 76 | 115.919 | 21.996 | 9.486 | 1.00 | 19.00 | C | |
| ATOM | 600 | CD1 | LEU | A | 76 | 115.760 | 23.199 | 10.381 | 1.00 | 19.83 | C | |
| ATOM | 601 | CD2 | LEU | A | 76 | 115.082 | 22.127 | 8.195 | 1.00 | 20.55 | C | |
| ATOM | 602 | N | LYS | A | 77 | 119.739 | 21.307 | 7.077 | 1.00 | 20.48 | N | |
| ATOM | 603 | CA | LYS | A | 77 | 121.181 | 21.419 | 6.838 | 1.00 | 21.33 | C | |
| ATOM | 604 | C | LYS | A | 77 | 121.513 | 22.919 | 6.845 | 1.00 | 20.52 | C | |
| ATOM | 605 | O | LYS | A | 77 | 120.930 | 23.687 | 6.104 | 1.00 | 23.43 | O | |
| ATOM | 606 | CB | LYS | A | 77 | 121.589 | 20.809 | 5.495 | 1.00 | 21.90 | C | |
| ATOM | 607 | CG | LYS | A | 77 | 123.091 | 20.908 | 5.269 | 1.00 | 27.81 | C | |
| ATOM | 608 | CD | LYS | A | 77 | 123.481 | 20.459 | 3.846 | 1.00 | 31.24 | C | |
| ATOM | 609 | CE | LYS | A | 77 | 123.157 | 19.006 | 3.616 | 1.00 | 33.01 | C | |
| ATOM | 610 | NZ | LYS | A | 77 | 124.105 | 18.139 | 4.371 | 1.00 | 30.19 | N | |
| ATOM | 611 | N | PHE | A | 78 | 122.403 | 23.340 | 7.744 | 1.00 | 20.17 | N | |
| ATOM | 612 | CA | PHE | A | 78 | 122.831 | 24.736 | 7.818 | 1.00 | 21.37 | C | |
| ATOM | 613 | C | PHE | A | 78 | 124.165 | 24.869 | 7.020 | 1.00 | 20.62 | C | |
| ATOM | 614 | O | PHE | A | 78 | 125.098 | 24.070 | 7.216 | 1.00 | 22.60 | O | |
| ATOM | 615 | CB | PHE | A | 78 | 123.085 | 25.123 | 9.284 | 1.00 | 20.29 | C | |
| ATOM | 616 | CG | PHE | A | 78 | 123.622 | 26.503 | 9.448 | 1.00 | 23.82 | C | |
| ATOM | 617 | CD1 | PHE | A | 78 | 122.887 | 27.595 | 9.023 | 1.00 | 26.69 | C | |
| ATOM | 618 | CD2 | PHE | A | 78 | 124.899 | 26.704 | 9.984 | 1.00 | 26.98 | C | |
| ATOM | 619 | CE1 | PHE | A | 78 | 123.393 | 28.907 | 9.106 | 1.00 | 28.56 | C | |
| ATOM | 620 | CE2 | PHE | A | 78 | 125.423 | 28.003 | 10.081 | 1.00 | 29.04 | C | |
| ATOM | 621 | CZ | PHE | A | 78 | 124.673 | 29.106 | 9.638 | 1.00 | 28.95 | C | |
| ATOM | 622 | N | ASER | A | 79 | 124.256 | 25.849 | 6.124 | 0.50 | 19.53 | N | |
| ATOM | 623 | N | BSER | A | 79 | 124.250 | 25.863 | 6.144 | 0.50 | 20.75 | N | |
| ATOM | 624 | CA | ASER | A | 79 | 125.488 | 26.013 | 5.340 | 0.50 | 19.78 | C | |
| ATOM | 625 | CA | BSER | A | 79 | 125.466 | 26.042 | 5.349 | 0.50 | 22.15 | C | |
| ATOM | 626 | C | ASER | A | 79 | 125.881 | 27.466 | 5.099 | 0.50 | 21.05 | C | |
| ATOM | 627 | C | BSER | A | 79 | 125.879 | 27.494 | 5.198 | 0.50 | 22.32 | C | |
| ATOM | 628 | O | ASER | A | 79 | 125.034 | 28.351 | 4.928 | 0.50 | 19.47 | O | |
| ATOM | 629 | O | BSER | A | 79 | 125.042 | 28.404 | 5.194 | 0.50 | 20.13 | O | |
| ATOM | 630 | CB | ASER | A | 79 | 125.383 | 25.296 | 3.990 | 0.50 | 20.28 | C | |
| ATOM | 631 | CB | BSER | A | 79 | 125.290 | 25.455 | 3.947 | 0.50 | 24.65 | C | |
| ATOM | 632 | OG | ASER | A | 79 | 124.439 | 25.932 | 3.148 | 0.50 | 14.83 | O | |
| ATOM | 633 | OG | BSER | A | 79 | 124.988 | 24.078 | 3.998 | 0.50 | 26.63 | O | |
| ATOM | 634 | N | ILE | A | 80 | 127.193 | 27.683 | 5.092 | 1.00 | 22.24 | N | |
| ATOM | 635 | CA | ILE | A | 80 | 127.794 | 28.978 | 4.868 | 1.00 | 24.19 | C | |
| ATOM | 636 | C | ILE | A | 80 | 128.715 | 28.696 | 3.709 | 1.00 | 24.41 | C | |
| ATOM | 637 | O | ILE | A | 80 | 129.588 | 27.842 | 3.772 | 1.00 | 24.11 | O | |
| ATOM | 638 | CB | ILE | A | 80 | 128.541 | 29.449 | 6.104 | 1.00 | 24.98 | C | |
| ATOM | 639 | CG1 | ILE | A | 80 | 127.516 | 29.886 | 7.145 | 1.00 | 26.04 | C | |
| ATOM | 640 | CG2 | ILE | A | 80 | 129.424 | 30.679 | 5.736 | 1.00 | 29.80 | C | |
| ATOM | 641 | CD1 | ILE | A | 80 | 128.093 | 30.071 | 8.571 | 1.00 | 27.58 | C | |
| ATOM | 642 | N | SER | A | 81 | 128.553 | 29.425 | 2.612 | 1.00 | 27.37 | N | |
| ATOM | 643 | CA | SER | A | 81 | 129.334 | 29.091 | 1.429 | 1.00 | 29.84 | C | |
| ATOM | 644 | C | SER | A | 81 | 130.668 | 29.687 | 1.057 | 1.00 | 33.15 | C | |
| ATOM | 645 | O | SER | A | 81 | 131.572 | 28.980 | 0.608 | 1.00 | 36.37 | O | |
| ATOM | 646 | CB | SER | A | 81 | 128.426 | 29.218 | 0.208 | 1.00 | 30.64 | C | |
| ATOM | 647 | OG | SER | A | 81 | 127.398 | 28.274 | 0.349 | 1.00 | 35.24 | O | |
| ATOM | 648 | N | ASN | A | 82 | 130.765 | 30.989 | 1.212 | 1.00 | 31.00 | N | |
| ATOM | 649 | CA | ASN | A | 82 | 131.949 | 31.726 | 0.793 | 1.00 | 32.50 | C | |
| ATOM | 650 | C | ASN | A | 82 | 132.363 | 32.705 | 1.880 | 1.00 | 28.11 | C | |
| ATOM | 651 | O | ASN | A | 82 | 132.411 | 33.918 | 1.658 | 1.00 | 29.86 | O | |
| ATOM | 652 | CB | ASN | A | 82 | 131.599 | 32.495 | −0.478 | 1.00 | 34.61 | C | |
| ATOM | 653 | CG | ASN | A | 82 | 132.783 | 33.182 | −1.060 | 1.00 | 44.42 | C | |
| ATOM | 654 | OD1 | ASN | A | 82 | 133.778 | 32.532 | −1.395 | 1.00 | 47.07 | O | |
| ATOM | 655 | ND2 | ASN | A | 82 | 132.704 | 34.511 | −1.183 | 1.00 | 44.52 | N | |
| ATOM | 656 | N | ALA | A | 83 | 132.634 | 32.166 | 3.061 | 1.00 | 24.41 | N | |
| ATOM | 657 | CA | ALA | A | 83 | 133.053 | 32.975 | 4.208 | 1.00 | 22.45 | C | |
| ATOM | 658 | C | ALA | A | 83 | 134.532 | 33.409 | 4.070 | 1.00 | 22.84 | C | |
| ATOM | 659 | O | ALA | A | 83 | 135.311 | 32.758 | 3.378 | 1.00 | 21.56 | O | |
| ATOM | 660 | CB | ALA | A | 83 | 132.879 | 32.173 | 5.506 | 1.00 | 20.04 | C | |
| ATOM | 661 | N | ASN | A | 84 | 134.904 | 34.518 | 4.699 | 1.00 | 23.80 | N | |
| ATOM | 662 | CA | ASN | A | 84 | 136.321 | 34.927 | 4.628 | 1.00 | 24.29 | C | |
| ATOM | 663 | C | ASN | A | 84 | 136.732 | 35.488 | 5.978 | 1.00 | 23.21 | C | |
| ATOM | 664 | O | ASN | A | 84 | 135.947 | 36.119 | 6.663 | 1.00 | 24.01 | O | |
| ATOM | 665 | CB | ASN | A | 84 | 136.595 | 35.934 | 3.478 | 1.00 | 30.16 | C | |
| ATOM | 666 | CG | ASN | A | 84 | 136.075 | 37.339 | 3.747 | 1.00 | 36.64 | C | |
| ATOM | 667 | OD1 | ASN | A | 84 | 136.657 | 38.096 | 4.533 | 1.00 | 42.08 | O | |
| ATOM | 668 | ND2 | ASN | A | 84 | 134.984 | 37.711 | 3.064 | 1.00 | 40.03 | N | |
| ATOM | 669 | N | ILE | A | 85 | 137.959 | 35.190 | 6.392 | 1.00 | 22.67 | N | |
| ATOM | 670 | CA | ILE | A | 85 | 138.407 | 35.650 | 7.696 | 1.00 | 22.26 | C | |
| ATOM | 671 | C | ILE | A | 85 | 139.610 | 36.557 | 7.493 | 1.00 | 25.49 | C | |
| ATOM | 672 | O | ILE | A | 85 | 140.452 | 36.256 | 6.677 | 1.00 | 22.03 | O | |
| ATOM | 673 | CB | ILE | A | 85 | 138.853 | 34.467 | 8.589 | 1.00 | 22.94 | C | |
| ATOM | 674 | CG1 | ILE | A | 85 | 137.831 | 33.322 | 8.499 | 1.00 | 26.02 | C | |
| ATOM | 675 | CG2 | ILE | A | 85 | 138.937 | 34.955 | 10.038 | 1.00 | 20.84 | C | |
| ATOM | 676 | CD1 | ILE | A | 85 | 138.247 | 32.007 | 9.128 | 1.00 | 24.20 | C | |
| ATOM | 677 | N | LYS | A | 86 | 139.640 | 37.683 | 8.199 | 1.00 | 23.67 | N | |
| ATOM | 678 | CA | LYS | A | 86 | 140.791 | 38.580 | 8.128 | 1.00 | 25.28 | C | |
| ATOM | 679 | C | LYS | A | 86 | 141.435 | 38.518 | 9.512 | 1.00 | 25.53 | C | |
| ATOM | 680 | O | LYS | A | 86 | 140.764 | 38.738 | 10.520 | 1.00 | 23.83 | O | |
| ATOM | 681 | CB | LYS | A | 86 | 140.326 | 39.997 | 7.808 | 1.00 | 27.02 | C | |
| ATOM | 682 | CG | LYS | A | 86 | 140.047 | 40.105 | 6.419 | 1.00 | 34.91 | C | |
| ATOM | 683 | N | ILE | A | 87 | 142.737 | 38.203 | 9.556 | 1.00 | 22.34 | N | |
| ATOM | 684 | CA | ILE | A | 87 | 143.453 | 38.064 | 10.816 | 1.00 | 22.43 | C | |
| ATOM | 685 | C | ILE | A | 87 | 144.691 | 38.950 | 10.797 | 1.00 | 22.61 | C | |
| ATOM | 686 | O | ILE | A | 87 | 145.310 | 39.132 | 9.748 | 1.00 | 22.28 | O | |
| ATOM | 687 | CB | ILE | A | 87 | 143.887 | 36.582 | 11.006 | 1.00 | 21.33 | C | |
| ATOM | 688 | CG1 | ILE | A | 87 | 142.668 | 35.718 | 11.363 | 1.00 | 22.96 | C | |
| ATOM | 689 | CG2 | ILE | A | 87 | 145.014 | 36.463 | 12.053 | 1.00 | 21.98 | C | |
| ATOM | 690 | CD1 | ILE | A | 87 | 142.871 | 34.234 | 10.985 | 1.00 | 25.39 | C | |
| ATOM | 691 | N | ASER | A | 88 | 145.015 | 39.555 | 11.949 | 0.50 | 23.16 | N | |
| ATOM | 692 | N | BSER | A | 88 | 145.046 | 39.497 | 11.949 | 0.50 | 23.70 | N | |
| ATOM | 693 | CA | ASER | A | 88 | 146.211 | 40.416 | 12.083 | 0.50 | 20.26 | C | |
| ATOM | 694 | CA | BSER | A | 88 | 146.264 | 40.282 | 12.005 | 0.50 | 21.49 | C | |
| ATOM | 695 | C | ASER | A | 88 | 147.072 | 39.844 | 13.219 | 0.50 | 20.40 | C | |
| ATOM | 696 | C | BSER | A | 88 | 147.098 | 39.584 | 13.068 | 0.50 | 20.77 | C | |
| ATOM | 697 | O | ASER | A | 88 | 146.539 | 39.471 | 14.247 | 0.50 | 19.73 | O | |
| ATOM | 698 | O | BSER | A | 88 | 146.584 | 38.813 | 13.859 | 0.50 | 22.55 | O | |
| ATOM | 699 | CB | ASER | A | 88 | 145.829 | 41.856 | 12.423 | 0.50 | 23.31 | C | |
| ATOM | 700 | CB | BSER | A | 88 | 145.985 | 41.719 | 12.413 | 0.50 | 23.70 | C | |
| ATOM | 701 | OG | ASER | A | 88 | 145.197 | 42.495 | 11.324 | 0.50 | 23.12 | O | |
| ATOM | 702 | OG | BSER | A | 88 | 145.294 | 41.763 | 13.639 | 0.50 | 26.12 | O | |
| ATOM | 703 | N | GLY | A | 89 | 148.391 | 39.830 | 13.040 | 1.00 | 20.01 | N | |
| ATOM | 704 | CA | GLY | A | 89 | 149.278 | 39.251 | 14.033 | 1.00 | 24.31 | C | |
| ATOM | 705 | C | GLY | A | 89 | 150.678 | 39.814 | 13.896 | 1.00 | 22.39 | C | |
| ATOM | 706 | O | GLY | A | 89 | 150.881 | 40.779 | 13.164 | 1.00 | 23.72 | O | |
| ATOM | 707 | N | LYS | A | 90 | 151.610 | 39.222 | 14.640 | 1.00 | 24.28 | N | |
| ATOM | 708 | CA | LYS | A | 90 | 153.008 | 39.641 | 14.663 | 1.00 | 23.69 | C | |
| ATOM | 709 | C | LYS | A | 90 | 153.855 | 38.479 | 14.165 | 1.00 | 23.77 | C | |
| ATOM | 710 | O | LYS | A | 90 | 153.516 | 37.312 | 14.343 | 1.00 | 21.80 | O | |
| ATOM | 711 | CB | LYS | A | 90 | 153.445 | 39.986 | 16.108 | 1.00 | 26.35 | C | |
| ATOM | 712 | CG | LYS | A | 90 | 152.579 | 41.041 | 16.870 | 1.00 | 31.57 | C | |
| ATOM | 713 | CD | LYS | A | 90 | 153.072 | 42.508 | 16.674 | 1.00 | 38.33 | C | |
| ATOM | 714 | CE | LYS | A | 90 | 152.315 | 43.489 | 17.582 | 1.00 | 42.59 | C | |
| ATOM | 715 | NZ | LYS | A | 90 | 152.324 | 43.104 | 19.067 | 1.00 | 43.06 | N | |
| ATOM | 716 | N | TRP | A | 91 | 154.973 | 38.805 | 13.522 | 1.00 | 21.76 | N | |
| ATOM | 717 | CA | TRP | A | 91 | 155.888 | 37.757 | 13.056 | 1.00 | 22.40 | C | |
| ATOM | 718 | C | TRP | A | 91 | 157.309 | 38.133 | 13.524 | 1.00 | 21.38 | C | |
| ATOM | 719 | O | TRP | A | 91 | 157.563 | 39.282 | 13.827 | 1.00 | 22.12 | O | |
| ATOM | 720 | CB | TRP | A | 91 | 155.844 | 37.655 | 11.509 | 1.00 | 21.28 | C | |
| ATOM | 721 | CG | TRP | A | 91 | 156.218 | 38.931 | 10.752 | 1.00 | 20.64 | C | |
| ATOM | 722 | CD1 | TRP | A | 91 | 155.382 | 39.941 | 10.354 | 1.00 | 18.50 | C | |
| ATOM | 723 | CD2 | TRP | A | 91 | 157.529 | 39.328 | 10.359 | 1.00 | 19.61 | C | |
| ATOM | 724 | NE1 | TRP | A | 91 | 156.085 | 40.934 | 9.749 | 1.00 | 18.60 | N | |
| ATOM | 725 | CE2 | TRP | A | 91 | 157.412 | 40.600 | 9.740 | 1.00 | 19.31 | C | |
| ATOM | 726 | CE3 | TRP | A | 91 | 158.799 | 38.738 | 10.476 | 1.00 | 22.70 | C | |
| ATOM | 727 | CZ2 | TRP | A | 91 | 158.507 | 41.293 | 9.241 | 1.00 | 20.25 | C | |
| ATOM | 728 | CZ3 | TRP | A | 91 | 159.897 | 39.433 | 9.978 | 1.00 | 23.65 | C | |
| ATOM | 729 | CH2 | TRP | A | 91 | 159.732 | 40.709 | 9.370 | 1.00 | 21.86 | C | |
| ATOM | 730 | N | LYS | A | 92 | 158.204 | 37.163 | 13.598 | 1.00 | 21.79 | N | |
| ATOM | 731 | CA | LYS | A | 92 | 159.609 | 37.469 | 13.944 | 1.00 | 20.93 | C | |
| ATOM | 732 | C | LYS | A | 92 | 160.455 | 36.355 | 13.351 | 1.00 | 21.03 | C | |
| ATOM | 733 | O | LYS | A | 92 | 159.972 | 35.227 | 13.146 | 1.00 | 21.06 | O | |
| ATOM | 734 | CB | LYS | A | 92 | 159.793 | 37.563 | 15.466 | 1.00 | 22.79 | C | |
| ATOM | 735 | CG | LYS | A | 92 | 159.551 | 36.240 | 16.194 | 1.00 | 25.27 | C | |
| ATOM | 736 | CD | LYS | A | 92 | 159.691 | 36.443 | 17.713 | 1.00 | 27.06 | C | |
| ATOM | 737 | CE | LYS | A | 92 | 159.589 | 35.079 | 18.389 | 1.00 | 30.68 | C | |
| ATOM | 738 | NZ | LYS | A | 92 | 159.675 | 35.232 | 19.852 | 1.00 | 38.44 | N | |
| ATOM | 739 | N | ALA | A | 93 | 161.694 | 36.662 | 12.968 | 1.00 | 18.15 | N | |
| ATOM | 740 | CA | ALA | A | 93 | 162.559 | 35.620 | 12.424 | 1.00 | 19.46 | C | |
| ATOM | 741 | C | ALA | A | 93 | 163.972 | 35.935 | 12.869 | 1.00 | 24.33 | C | |
| ATOM | 742 | O | ALA | A | 93 | 164.306 | 37.094 | 13.138 | 1.00 | 24.02 | O | |
| ATOM | 743 | CB | ALA | A | 93 | 162.515 | 35.569 | 10.883 | 1.00 | 21.11 | C | |
| ATOM | 744 | N | GLN | A | 94 | 164.802 | 34.896 | 12.934 | 1.00 | 22.39 | N | |
| ATOM | 745 | CA | GLN | A | 94 | 166.175 | 35.100 | 13.357 | 1.00 | 26.59 | C | |
| ATOM | 746 | C | GLN | A | 94 | 167.136 | 34.197 | 12.602 | 1.00 | 22.65 | C | |
| ATOM | 747 | O | GLN | A | 94 | 166.860 | 33.027 | 12.371 | 1.00 | 25.68 | O | |
| ATOM | 748 | CB | GLN | A | 94 | 166.314 | 34.891 | 14.884 | 1.00 | 29.32 | C | |
| ATOM | 749 | CG | GLN | A | 94 | 167.709 | 35.262 | 15.392 | 1.00 | 36.01 | C | |
| ATOM | 750 | CD | GLN | A | 94 | 167.775 | 35.561 | 16.895 | 1.00 | 43.53 | C | |
| ATOM | 751 | OE1 | GLN | A | 94 | 166.767 | 35.478 | 17.620 | 1.00 | 46.22 | O | |
| ATOM | 752 | NE2 | GLN | A | 94 | 168.975 | 35.912 | 17.370 | 1.00 | 44.81 | N | |
| ATOM | 753 | N | LYS | A | 95 | 168.273 | 34.779 | 12.212 | 1.00 | 24.42 | N | |
| ATOM | 754 | CA | LYS | A | 95 | 169.315 | 34.075 | 11.484 | 1.00 | 21.97 | C | |
| ATOM | 755 | C | LYS | A | 95 | 170.602 | 34.619 | 12.138 | 1.00 | 25.33 | C | |
| ATOM | 756 | O | LYS | A | 95 | 170.953 | 35.800 | 11.985 | 1.00 | 24.33 | O | |
| ATOM | 757 | CB | LYS | A | 95 | 169.283 | 34.440 | 9.976 | 1.00 | 26.58 | C | |
| ATOM | 758 | N | ARG | A | 96 | 171.264 | 33.749 | 12.873 | 1.00 | 24.94 | N | |
| ATOM | 759 | CA | ARG | A | 96 | 172.474 | 34.109 | 13.615 | 1.00 | 24.07 | C | |
| ATOM | 760 | C | ARG | A | 96 | 172.075 | 35.229 | 14.589 | 1.00 | 23.00 | C | |
| ATOM | 761 | O | ARG | A | 96 | 171.250 | 35.008 | 15.480 | 1.00 | 24.39 | O | |
| ATOM | 762 | CB | ARG | A | 96 | 173.605 | 34.523 | 12.669 | 1.00 | 25.93 | C | |
| ATOM | 763 | CG | ARG | A | 96 | 174.970 | 34.502 | 13.361 | 1.00 | 27.66 | C | |
| ATOM | 764 | CD | ARG | A | 96 | 176.185 | 34.577 | 12.396 | 1.00 | 29.73 | C | |
| ATOM | 765 | NE | ARG | A | 96 | 177.406 | 34.891 | 13.153 | 1.00 | 21.21 | N | |
| ATOM | 766 | CZ | ARG | A | 96 | 178.031 | 34.033 | 13.974 | 1.00 | 27.35 | C | |
| ATOM | 767 | NH1 | ARG | A | 96 | 177.585 | 32.784 | 14.144 | 1.00 | 27.00 | N | |
| ATOM | 768 | NH2 | ARG | A | 96 | 179.098 | 34.420 | 14.670 | 1.00 | 22.37 | N | |
| ATOM | 769 | N | PHE | A | 97 | 172.619 | 36.427 | 14.422 | 1.00 | 22.63 | N | |
| ATOM | 770 | CA | PHE | A | 97 | 172.291 | 37.501 | 15.371 | 1.00 | 23.86 | C | |
| ATOM | 771 | C | PHE | A | 97 | 171.279 | 38.480 | 14.815 | 1.00 | 23.28 | C | |
| ATOM | 772 | O | PHE | A | 97 | 170.825 | 39.378 | 15.534 | 1.00 | 25.98 | O | |
| ATOM | 773 | CB | PHE | A | 97 | 173.560 | 38.281 | 15.738 | 1.00 | 22.00 | C | |
| ATOM | 774 | CG | PHE | A | 97 | 174.589 | 37.460 | 16.511 | 1.00 | 22.50 | C | |
| ATOM | 775 | CD1 | PHE | A | 97 | 174.321 | 36.988 | 17.802 | 1.00 | 26.36 | C | |
| ATOM | 776 | CD2 | PHE | A | 97 | 175.834 | 37.204 | 15.956 | 1.00 | 21.67 | C | |
| ATOM | 777 | CE1 | PHE | A | 97 | 175.295 | 36.274 | 18.508 | 1.00 | 25.83 | C | |
| ATOM | 778 | CE2 | PHE | A | 97 | 176.812 | 36.509 | 16.635 | 1.00 | 22.10 | C | |
| ATOM | 779 | CZ | PHE | A | 97 | 176.548 | 36.034 | 17.923 | 1.00 | 21.99 | C | |
| ATOM | 780 | N | LEU | A | 98 | 170.932 | 38.306 | 13.549 | 1.00 | 23.41 | N | |
| ATOM | 781 | CA | LEU | A | 98 | 169.990 | 39.239 | 12.917 | 1.00 | 26.32 | C | |
| ATOM | 782 | C | LEU | A | 98 | 168.559 | 38.925 | 13.351 | 1.00 | 27.35 | C | |
| ATOM | 783 | O | LEU | A | 98 | 168.113 | 37.787 | 13.240 | 1.00 | 30.10 | O | |
| ATOM | 784 | CB | LEU | A | 98 | 170.143 | 39.139 | 11.407 | 1.00 | 25.13 | C | |
| ATOM | 785 | CG | LEU | A | 98 | 169.076 | 39.838 | 10.576 | 1.00 | 31.23 | C | |
| ATOM | 786 | CD1 | LEU | A | 98 | 169.396 | 41.354 | 10.436 | 1.00 | 30.01 | C | |
| ATOM | 787 | CD2 | LEU | A | 98 | 169.046 | 39.148 | 9.209 | 1.00 | 26.53 | C | |
| ATOM | 788 | N | LYS | A | 99 | 167.868 | 39.935 | 13.870 | 1.00 | 25.74 | N | |
| ATOM | 789 | CA | LYS | A | 99 | 166.501 | 39.783 | 14.338 | 1.00 | 27.25 | C | |
| ATOM | 790 | C | LYS | A | 99 | 165.617 | 40.623 | 13.416 | 1.00 | 30.30 | C | |
| ATOM | 791 | O | LYS | A | 99 | 165.926 | 41.776 | 13.119 | 1.00 | 30.57 | O | |
| ATOM | 792 | CB | LYS | A | 99 | 166.384 | 40.237 | 15.764 | 1.00 | 25.35 | C | |
| ATOM | 793 | N | MET | A | 100 | 164.545 | 40.009 | 12.917 | 1.00 | 29.66 | N | |
| ATOM | 794 | CA | MET | A | 100 | 163.618 | 40.700 | 12.019 | 1.00 | 28.99 | C | |
| ATOM | 795 | C | MET | A | 100 | 162.248 | 40.559 | 12.658 | 1.00 | 27.42 | C | |
| ATOM | 796 | O | MET | A | 100 | 161.983 | 39.563 | 13.299 | 1.00 | 27.29 | O | |
| ATOM | 797 | CB | MET | A | 100 | 163.633 | 40.030 | 10.645 | 1.00 | 29.51 | C | |
| ATOM | 798 | CG | MET | A | 100 | 165.063 | 39.802 | 10.114 | 1.00 | 41.07 | C | |
| ATOM | 799 | SD | MET | A | 100 | 165.162 | 39.262 | 8.395 | 1.00 | 46.05 | S | |
| ATOM | 800 | CE | MET | A | 100 | 164.652 | 40.890 | 7.678 | 1.00 | 40.34 | C | |
| ATOM | 801 | N | SER | A | 101 | 161.378 | 41.556 | 12.507 | 1.00 | 23.45 | N | |
| ATOM | 802 | CA | SER | A | 101 | 160.056 | 41.423 | 13.116 | 1.00 | 24.63 | C | |
| ATOM | 803 | C | SER | A | 101 | 159.082 | 42.436 | 12.502 | 1.00 | 23.55 | C | |
| ATOM | 804 | O | SER | A | 101 | 159.502 | 43.403 | 11.852 | 1.00 | 24.07 | O | |
| ATOM | 805 | CB | SER | A | 101 | 160.133 | 41.663 | 14.632 | 1.00 | 27.71 | C | |
| ATOM | 806 | OG | SER | A | 101 | 160.421 | 43.024 | 14.919 | 1.00 | 30.69 | O | |
| ATOM | 807 | N | GLY | A | 102 | 157.789 | 42.230 | 12.755 | 1.00 | 22.08 | N | |
| ATOM | 808 | CA | GLY | A | 102 | 156.804 | 43.151 | 12.209 | 1.00 | 23.14 | C | |
| ATOM | 809 | C | GLY | A | 102 | 155.380 | 42.658 | 12.371 | 1.00 | 22.83 | C | |
| ATOM | 810 | O | GLY | A | 102 | 155.102 | 41.809 | 13.207 | 1.00 | 22.38 | O | |
| ATOM | 811 | N | ASN | A | 103 | 154.469 | 43.231 | 11.586 | 1.00 | 23.46 | N | |
| ATOM | 812 | CA | ASN | A | 103 | 153.061 | 42.825 | 11.614 | 1.00 | 22.49 | C | |
| ATOM | 813 | C | ASN | A | 103 | 152.747 | 42.049 | 10.322 | 1.00 | 20.64 | C | |
| ATOM | 814 | O | ASN | A | 103 | 153.428 | 42.176 | 9.299 | 1.00 | 19.15 | O | |
| ATOM | 815 | CB | ASN | A | 103 | 152.111 | 44.043 | 11.622 | 1.00 | 24.68 | C | |
| ATOM | 816 | CG | ASN | A | 103 | 152.284 | 44.936 | 12.840 | 1.00 | 31.18 | C | |
| ATOM | 817 | OD1 | ASN | A | 103 | 152.391 | 46.159 | 12.704 | 1.00 | 36.34 | O | |
| ATOM | 818 | ND2 | ASN | A | 103 | 152.297 | 44.339 | 14.037 | 1.00 | 29.64 | N | |
| ATOM | 819 | N | PHE | A | 104 | 151.696 | 41.243 | 10.384 | 1.00 | 20.66 | N | |
| ATOM | 820 | CA | PHE | A | 104 | 151.242 | 40.569 | 9.151 | 1.00 | 19.34 | C | |
| ATOM | 821 | C | PHE | A | 104 | 149.713 | 40.497 | 9.171 | 1.00 | 21.52 | C | |
| ATOM | 822 | O | PHE | A | 104 | 149.072 | 40.588 | 10.220 | 1.00 | 20.97 | O | |
| ATOM | 823 | CB | PHE | A | 104 | 151.823 | 39.156 | 8.954 | 1.00 | 18.61 | C | |
| ATOM | 824 | CG | PHE | A | 104 | 151.249 | 38.112 | 9.889 | 1.00 | 20.84 | C | |
| ATOM | 825 | CD1 | PHE | A | 104 | 151.781 | 37.931 | 11.174 | 1.00 | 22.35 | C | |
| ATOM | 826 | CD2 | PHE | A | 104 | 150.203 | 37.293 | 9.495 | 1.00 | 24.22 | C | |
| ATOM | 827 | CE1 | PHE | A | 104 | 151.273 | 36.951 | 12.038 | 1.00 | 19.71 | C | |
| ATOM | 828 | CE2 | PHE | A | 104 | 149.671 | 36.285 | 10.356 | 1.00 | 20.69 | C | |
| ATOM | 829 | CZ | PHE | A | 104 | 150.209 | 36.118 | 11.631 | 1.00 | 22.89 | C | |
| ATOM | 830 | N | ASP | A | 105 | 149.135 | 40.362 | 7.978 | 1.00 | 23.74 | N | |
| ATOM | 831 | CA | ASP | A | 105 | 147.689 | 40.204 | 7.828 | 1.00 | 20.72 | C | |
| ATOM | 832 | C | ASP | A | 105 | 147.527 | 38.895 | 7.096 | 1.00 | 21.31 | C | |
| ATOM | 833 | O | ASP | A | 105 | 148.220 | 38.614 | 6.122 | 1.00 | 20.54 | O | |
| ATOM | 834 | CB | ASP | A | 105 | 147.063 | 41.275 | 6.935 | 1.00 | 28.57 | C | |
| ATOM | 835 | CG | ASP | A | 105 | 147.355 | 42.629 | 7.415 | 1.00 | 35.98 | C | |
| ATOM | 836 | OD1 | ASP | A | 105 | 147.144 | 42.866 | 8.638 | 1.00 | 37.75 | O | |
| ATOM | 837 | OD2 | ASP | A | 105 | 147.807 | 43.437 | 6.576 | 1.00 | 41.46 | O | |
| ATOM | 838 | N | LEU | A | 106 | 146.565 | 38.113 | 7.541 | 1.00 | 21.47 | N | |
| ATOM | 839 | CA | LEU | A | 106 | 146.313 | 36.822 | 6.925 | 1.00 | 22.72 | C | |
| ATOM | 840 | C | LEU | A | 106 | 144.860 | 36.844 | 6.442 | 1.00 | 24.68 | C | |
| ATOM | 841 | O | LEU | A | 106 | 143.964 | 37.296 | 7.180 | 1.00 | 24.55 | O | |
| ATOM | 842 | CB | LEU | A | 106 | 146.505 | 35.763 | 7.988 | 1.00 | 24.34 | C | |
| ATOM | 843 | CG | LEU | A | 106 | 146.540 | 34.270 | 7.663 | 1.00 | 32.41 | C | |
| ATOM | 844 | CD1 | LEU | A | 106 | 146.694 | 33.570 | 9.003 | 1.00 | 29.80 | C | |
| ATOM | 845 | CD2 | LEU | A | 106 | 145.316 | 33.771 | 6.946 | 1.00 | 35.99 | C | |
| ATOM | 846 | N | ASER | A | 107 | 144.637 | 36.378 | 5.211 | 0.50 | 21.67 | N | |
| ATOM | 847 | N | BSER | A | 107 | 144.632 | 36.374 | 5.217 | 0.50 | 22.18 | N | |
| ATOM | 848 | CA | ASER | A | 107 | 143.305 | 36.321 | 4.622 | 0.50 | 21.78 | C | |
| ATOM | 849 | CA | BSER | A | 107 | 143.289 | 36.320 | 4.660 | 0.50 | 22.50 | C | |
| ATOM | 850 | C | ASER | A | 107 | 142.947 | 34.861 | 4.333 | 0.50 | 21.97 | C | |
| ATOM | 851 | C | BSER | A | 107 | 142.947 | 34.867 | 4.342 | 0.50 | 22.41 | C | |
| ATOM | 852 | O | ASER | A | 107 | 143.645 | 34.208 | 3.555 | 0.50 | 21.66 | O | |
| ATOM | 853 | O | BSER | A | 107 | 143.651 | 34.227 | 3.558 | 0.50 | 21.93 | O | |
| ATOM | 854 | CB | ASER | A | 107 | 143.286 | 37.114 | 3.310 | 0.50 | 23.18 | C | |
| ATOM | 855 | CB | BSER | A | 107 | 143.224 | 37.158 | 3.384 | 0.50 | 24.66 | C | |
| ATOM | 856 | OG | ASER | A | 107 | 143.570 | 38.490 | 3.516 | 0.50 | 24.24 | O | |
| ATOM | 857 | OG | BSER | A | 107 | 141.926 | 37.104 | 2.816 | 0.50 | 26.75 | O | |
| ATOM | 858 | N | ILE | A | 108 | 141.890 | 34.344 | 4.966 | 1.00 | 21.20 | N | |
| ATOM | 859 | CA | ILE | A | 108 | 141.452 | 32.979 | 4.723 | 1.00 | 21.64 | C | |
| ATOM | 860 | C | ILE | A | 108 | 140.218 | 33.194 | 3.842 | 1.00 | 21.69 | C | |
| ATOM | 861 | O | ILE | A | 108 | 139.250 | 33.852 | 4.275 | 1.00 | 23.36 | O | |
| ATOM | 862 | CB | ILE | A | 108 | 141.132 | 32.237 | 6.037 | 1.00 | 23.74 | C | |
| ATOM | 863 | CG1 | ILE | A | 108 | 142.468 | 32.018 | 6.798 | 1.00 | 25.03 | C | |
| ATOM | 864 | CG2 | ILE | A | 108 | 140.575 | 30.833 | 5.766 | 1.00 | 24.19 | C | |
| ATOM | 865 | CD1 | ILE | A | 108 | 142.320 | 31.512 | 8.213 | 1.00 | 27.18 | C | |
| ATOM | 866 | N | GLU | A | 109 | 140.279 | 32.640 | 2.635 | 1.00 | 23.05 | N | |
| ATOM | 867 | CA | GLU | A | 109 | 139.251 | 32.850 | 1.626 | 1.00 | 23.47 | C | |
| ATOM | 868 | C | GLU | A | 109 | 138.484 | 31.618 | 1.179 | 1.00 | 22.66 | C | |
| ATOM | 869 | O | GLU | A | 109 | 138.991 | 30.506 | 1.188 | 1.00 | 20.85 | O | |
| ATOM | 870 | CB | GLU | A | 109 | 139.878 | 33.496 | 0.372 | 1.00 | 27.15 | C | |
| ATOM | 871 | CG | GLU | A | 109 | 140.425 | 34.931 | 0.572 | 1.00 | 34.19 | C | |
| ATOM | 872 | CD | GLU | A | 109 | 139.360 | 36.044 | 0.478 | 1.00 | 43.20 | C | |
| ATOM | 873 | OE1 | GLU | A | 109 | 138.199 | 35.776 | 0.077 | 1.00 | 46.70 | O | |
| ATOM | 874 | OE2 | GLU | A | 109 | 139.694 | 37.216 | 0.787 | 1.00 | 45.75 | O | |
| ATOM | 875 | N | GLY | A | 110 | 137.232 | 31.846 | 0.808 | 1.00 | 22.53 | N | |
| ATOM | 876 | CA | GLY | A | 110 | 136.442 | 30.731 | 0.305 | 1.00 | 25.28 | C | |
| ATOM | 877 | C | GLY | A | 110 | 136.208 | 29.591 | 1.277 | 1.00 | 24.79 | C | |
| ATOM | 878 | O | GLY | A | 110 | 136.309 | 28.407 | 0.886 | 1.00 | 23.81 | O | |
| ATOM | 879 | N | MET | A | 111 | 135.833 | 29.921 | 2.514 | 1.00 | 23.49 | N | |
| ATOM | 880 | CA | MET | A | 111 | 135.605 | 28.882 | 3.520 | 1.00 | 21.00 | C | |
| ATOM | 881 | C | MET | A | 111 | 134.153 | 28.414 | 3.437 | 1.00 | 22.68 | C | |
| ATOM | 882 | O | MET | A | 111 | 133.233 | 29.232 | 3.356 | 1.00 | 24.61 | O | |
| ATOM | 883 | CB | MET | A | 111 | 135.880 | 29.445 | 4.937 | 1.00 | 20.87 | C | |
| ATOM | 884 | CG | MET | A | 111 | 135.404 | 28.576 | 6.061 | 1.00 | 27.11 | C | |
| ATOM | 885 | SD | MET | A | 111 | 135.830 | 29.331 | 7.704 | 1.00 | 30.86 | S | |
| ATOM | 886 | CE | MET | A | 111 | 137.268 | 28.413 | 8.209 | 1.00 | 25.88 | C | |
| ATOM | 887 | N | SER | A | 112 | 133.949 | 27.100 | 3.428 | 1.00 | 17.62 | N | |
| ATOM | 888 | CA | SER | A | 112 | 132.569 | 26.616 | 3.393 | 1.00 | 22.22 | C | |
| ATOM | 889 | C | SER | A | 112 | 132.347 | 25.777 | 4.652 | 1.00 | 21.56 | C | |
| ATOM | 890 | O | SER | A | 112 | 133.193 | 24.959 | 5.017 | 1.00 | 22.01 | O | |
| ATOM | 891 | CB | SER | A | 112 | 132.288 | 25.808 | 2.113 | 1.00 | 27.44 | C | |
| ATOM | 892 | OG | SER | A | 112 | 133.274 | 24.825 | 1.876 | 1.00 | 32.30 | O | |
| ATOM | 893 | N | ILE | A | 113 | 131.222 | 26.007 | 5.308 | 1.00 | 22.11 | N | |
| ATOM | 894 | CA | ILE | A | 113 | 130.891 | 25.297 | 6.535 | 1.00 | 20.66 | C | |
| ATOM | 895 | C | ILE | A | 113 | 129.512 | 24.644 | 6.409 | 1.00 | 24.58 | C | |
| ATOM | 896 | O | ILE | A | 113 | 128.568 | 25.229 | 5.795 | 1.00 | 22.13 | O | |
| ATOM | 897 | CB | ILE | A | 113 | 130.854 | 26.312 | 7.717 | 1.00 | 24.08 | C | |
| ATOM | 898 | CG1 | ILE | A | 113 | 132.189 | 27.062 | 7.827 | 1.00 | 22.92 | C | |
| ATOM | 899 | CG2 | ILE | A | 113 | 130.563 | 25.585 | 9.037 | 1.00 | 25.41 | C | |
| ATOM | 900 | CD1 | ILE | A | 113 | 132.086 | 28.362 | 8.621 | 1.00 | 30.36 | C | |
| ATOM | 901 | N | SER | A | 114 | 129.376 | 23.426 | 6.917 | 1.00 | 20.55 | N | |
| ATOM | 902 | CA | SER | A | 114 | 128.016 | 22.861 | 6.864 | 1.00 | 25.37 | C | |
| ATOM | 903 | C | SER | A | 114 | 127.736 | 21.944 | 8.060 | 1.00 | 27.04 | C | |
| ATOM | 904 | O | SER | A | 114 | 128.610 | 21.194 | 8.523 | 1.00 | 24.97 | O | |
| ATOM | 905 | CB | SER | A | 114 | 127.743 | 22.160 | 5.525 | 1.00 | 29.07 | C | |
| ATOM | 906 | OG | SER | A | 114 | 128.578 | 21.059 | 5.330 | 1.00 | 37.22 | O | |
| ATOM | 907 | N | ALA | A | 115 | 126.510 | 22.024 | 8.570 | 1.00 | 24.26 | N | |
| ATOM | 908 | CA | ALA | A | 115 | 126.120 | 21.250 | 9.750 | 1.00 | 24.95 | C | |
| ATOM | 909 | C | ALA | A | 115 | 124.722 | 20.692 | 9.623 | 1.00 | 26.40 | C | |
| ATOM | 910 | O | ALA | A | 115 | 123.787 | 21.443 | 9.268 | 1.00 | 24.48 | O | |
| ATOM | 911 | CB | ALA | A | 115 | 126.199 | 22.126 | 10.977 | 1.00 | 24.78 | C | |
| ATOM | 912 | N | ASP | A | 116 | 124.580 | 19.401 | 9.938 | 1.00 | 22.64 | N | |
| ATOM | 913 | CA | ASP | A | 116 | 123.269 | 18.736 | 9.858 | 1.00 | 22.98 | C | |
| ATOM | 914 | C | ASP | A | 116 | 122.568 | 18.819 | 11.217 | 1.00 | 22.09 | C | |
| ATOM | 915 | O | ASP | A | 116 | 123.178 | 18.537 | 12.254 | 1.00 | 23.22 | O | |
| ATOM | 916 | CB | ASP | A | 116 | 123.429 | 17.263 | 9.421 | 1.00 | 25.84 | C | |
| ATOM | 917 | CG | ASP | A | 116 | 123.950 | 17.117 | 7.974 | 1.00 | 32.10 | C | |
| ATOM | 918 | OD1 | ASP | A | 116 | 123.578 | 17.904 | 7.079 | 1.00 | 31.96 | O | |
| ATOM | 919 | OD2 | ASP | A | 116 | 124.729 | 16.188 | 7.727 | 1.00 | 30.75 | O | |
| ATOM | 920 | N | LEU | A | 117 | 121.296 | 19.225 | 11.211 | 1.00 | 20.41 | N | |
| ATOM | 921 | CA | LEU | A | 117 | 120.546 | 19.386 | 12.448 | 1.00 | 21.52 | C | |
| ATOM | 922 | C | LEU | A | 117 | 119.379 | 18.392 | 12.479 | 1.00 | 23.01 | C | |
| ATOM | 923 | O | LEU | A | 117 | 118.590 | 18.343 | 11.544 | 1.00 | 23.43 | O | |
| ATOM | 924 | CB | LEU | A | 117 | 120.046 | 20.841 | 12.540 | 1.00 | 20.68 | C | |
| ATOM | 925 | CG | LEU | A | 117 | 121.066 | 21.970 | 12.337 | 1.00 | 23.24 | C | |
| ATOM | 926 | CD1 | LEU | A | 117 | 120.381 | 23.356 | 12.335 | 1.00 | 22.19 | C | |
| ATOM | 927 | CD2 | LEU | A | 117 | 122.073 | 21.905 | 13.510 | 1.00 | 25.64 | C | |
| ATOM | 928 | N | LYS | A | 118 | 119.321 | 17.549 | 13.519 | 1.00 | 21.80 | N | |
| ATOM | 929 | CA | LYS | A | 118 | 118.227 | 16.557 | 13.663 | 1.00 | 19.22 | C | |
| ATOM | 930 | C | LYS | A | 118 | 117.261 | 17.199 | 14.650 | 1.00 | 19.99 | C | |
| ATOM | 931 | O | LYS | A | 118 | 117.669 | 17.641 | 15.745 | 1.00 | 18.76 | O | |
| ATOM | 932 | CB | LYS | A | 118 | 118.780 | 15.172 | 14.283 | 1.00 | 21.67 | C | |
| ATOM | 933 | N | LEU | A | 119 | 116.007 | 17.309 | 14.234 | 1.00 | 22.60 | N | |
| ATOM | 934 | CA | LEU | A | 119 | 114.961 | 17.902 | 15.072 | 1.00 | 23.30 | C | |
| ATOM | 935 | C | LEU | A | 119 | 114.130 | 16.780 | 15.685 | 1.00 | 24.97 | C | |
| ATOM | 936 | O | LEU | A | 119 | 113.818 | 15.800 | 14.999 | 1.00 | 27.05 | O | |
| ATOM | 937 | CB | LEU | A | 119 | 114.025 | 18.746 | 14.214 | 1.00 | 22.29 | C | |
| ATOM | 938 | CG | LEU | A | 119 | 114.358 | 20.201 | 13.831 | 1.00 | 33.08 | C | |
| ATOM | 939 | CD1 | LEU | A | 119 | 115.807 | 20.442 | 13.626 | 1.00 | 28.79 | C | |
| ATOM | 940 | CD2 | LEU | A | 119 | 113.558 | 20.528 | 12.527 | 1.00 | 30.33 | C | |
| ATOM | 941 | N | GLY | A | 120 | 113.734 | 16.939 | 16.942 | 1.00 | 21.57 | N | |
| ATOM | 942 | CA | GLY | A | 120 | 112.921 | 15.914 | 17.576 | 1.00 | 20.39 | C | |
| ATOM | 943 | C | GLY | A | 120 | 112.024 | 16.523 | 18.645 | 1.00 | 25.82 | C | |
| ATOM | 944 | O | GLY | A | 120 | 111.875 | 17.750 | 18.727 | 1.00 | 24.69 | O | |
| ATOM | 945 | N | SER | A | 121 | 111.399 | 15.671 | 19.449 | 1.00 | 22.61 | N | |
| ATOM | 946 | CA | SER | A | 121 | 110.546 | 16.153 | 20.548 | 1.00 | 25.90 | C | |
| ATOM | 947 | C | SER | A | 121 | 110.669 | 15.184 | 21.733 | 1.00 | 29.24 | C | |
| ATOM | 948 | O | SER | A | 121 | 110.905 | 13.996 | 21.526 | 1.00 | 28.90 | O | |
| ATOM | 949 | CB | SER | A | 121 | 109.083 | 16.185 | 20.122 | 1.00 | 24.04 | C | |
| ATOM | 950 | OG | SER | A | 121 | 108.582 | 14.875 | 19.870 | 1.00 | 27.94 | O | |
| ATOM | 951 | N | ASN | A | 122 | 110.524 | 15.704 | 22.953 | 1.00 | 32.34 | N | |
| ATOM | 952 | CA | ASN | A | 122 | 110.533 | 14.889 | 24.168 | 1.00 | 35.84 | C | |
| ATOM | 953 | C | ASN | A | 122 | 109.033 | 14.641 | 24.406 | 1.00 | 33.91 | C | |
| ATOM | 954 | O | ASN | A | 122 | 108.291 | 15.545 | 24.817 | 1.00 | 30.71 | O | |
| ATOM | 955 | CB | ASN | A | 122 | 111.147 | 15.682 | 25.307 | 1.00 | 37.83 | C | |
| ATOM | 956 | CG | ASN | A | 122 | 111.239 | 14.875 | 26.572 | 1.00 | 41.22 | C | |
| ATOM | 957 | OD1 | ASN | A | 122 | 110.278 | 14.199 | 26.957 | 1.00 | 42.97 | O | |
| ATOM | 958 | ND2 | ASN | A | 122 | 112.393 | 14.933 | 27.229 | 1.00 | 39.72 | N | |
| ATOM | 959 | N | PRO | A | 123 | 108.568 | 13.407 | 24.177 | 1.00 | 33.73 | N | |
| ATOM | 960 | CA | PRO | A | 123 | 107.136 | 13.051 | 24.331 | 1.00 | 33.66 | C | |
| ATOM | 961 | C | PRO | A | 123 | 106.423 | 13.294 | 25.670 | 1.00 | 36.42 | C | |
| ATOM | 962 | O | PRO | A | 123 | 105.212 | 13.534 | 25.715 | 1.00 | 35.65 | O | |
| ATOM | 963 | CB | PRO | A | 123 | 107.080 | 11.566 | 23.926 | 1.00 | 35.36 | C | |
| ATOM | 964 | CG | PRO | A | 123 | 108.453 | 11.217 | 23.432 | 1.00 | 34.69 | C | |
| ATOM | 965 | CD | PRO | A | 123 | 109.404 | 12.198 | 24.075 | 1.00 | 35.39 | C | |
| ATOM | 966 | N | THR | A | 124 | 107.183 | 13.216 | 26.744 | 1.00 | 34.58 | N | |
| ATOM | 967 | CA | THR | A | 124 | 106.648 | 13.425 | 28.076 | 1.00 | 38.05 | C | |
| ATOM | 968 | C | THR | A | 124 | 106.471 | 14.938 | 28.360 | 1.00 | 35.63 | C | |
| ATOM | 969 | O | THR | A | 124 | 105.428 | 15.365 | 28.835 | 1.00 | 38.20 | O | |
| ATOM | 970 | CB | THR | A | 124 | 107.612 | 12.847 | 29.151 | 1.00 | 36.82 | C | |
| ATOM | 971 | OG1 | THR | A | 124 | 108.760 | 13.696 | 29.241 | 1.00 | 47.77 | O | |
| ATOM | 972 | CG2 | THR | A | 124 | 108.104 | 11.464 | 28.778 | 1.00 | 40.33 | C | |
| ATOM | 973 | N | SER | A | 125 | 107.491 | 15.733 | 28.060 | 1.00 | 34.06 | N | |
| ATOM | 974 | CA | SER | A | 125 | 107.442 | 17.178 | 28.352 | 1.00 | 30.51 | C | |
| ATOM | 975 | C | SER | A | 125 | 106.802 | 18.047 | 27.269 | 1.00 | 29.87 | C | |
| ATOM | 976 | O | SER | A | 125 | 106.440 | 19.209 | 27.537 | 1.00 | 26.13 | O | |
| ATOM | 977 | CB | SER | A | 125 | 108.856 | 17.711 | 28.605 | 1.00 | 32.19 | C | |
| ATOM | 978 | OG | SER | A | 125 | 109.641 | 17.724 | 27.421 | 1.00 | 30.08 | O | |
| ATOM | 979 | N | GLY | A | 126 | 106.652 | 17.493 | 26.060 | 1.00 | 23.48 | N | |
| ATOM | 980 | CA | GLY | A | 126 | 106.097 | 18.264 | 24.950 | 1.00 | 21.99 | C | |
| ATOM | 981 | C | GLY | A | 126 | 107.063 | 19.341 | 24.441 | 1.00 | 21.52 | C | |
| ATOM | 982 | O | GLY | A | 126 | 106.653 | 20.272 | 23.742 | 1.00 | 17.73 | O | |
| ATOM | 983 | N | LYS | A | 127 | 108.356 | 19.225 | 24.765 | 1.00 | 21.71 | N | |
| ATOM | 984 | CA | LYS | A | 127 | 109.345 | 20.229 | 24.334 | 1.00 | 25.04 | C | |
| ATOM | 985 | C | LYS | A | 127 | 110.155 | 19.745 | 23.119 | 1.00 | 25.34 | C | |
| ATOM | 986 | O | LYS | A | 127 | 110.351 | 18.556 | 22.960 | 1.00 | 25.65 | O | |
| ATOM | 987 | CB | LYS | A | 127 | 110.314 | 20.567 | 25.496 | 1.00 | 24.59 | C | |
| ATOM | 988 | CG | LYS | A | 127 | 109.598 | 21.084 | 26.775 | 1.00 | 25.53 | C | |
| ATOM | 989 | CD | LYS | A | 127 | 110.562 | 21.440 | 27.927 | 1.00 | 28.28 | C | |
| ATOM | 990 | CE | LYS | A | 127 | 111.233 | 22.797 | 27.741 | 1.00 | 31.14 | C | |
| ATOM | 991 | NZ | LYS | A | 127 | 110.210 | 23.878 | 27.743 | 1.00 | 28.86 | N | |
| ATOM | 992 | N | PRO | A | 128 | 110.594 | 20.668 | 22.243 | 1.00 | 24.84 | N | |
| ATOM | 993 | CA | PRO | A | 128 | 111.393 | 20.336 | 21.049 | 1.00 | 24.56 | C | |
| ATOM | 994 | C | PRO | A | 128 | 112.870 | 20.023 | 21.367 | 1.00 | 23.01 | C | |
| ATOM | 995 | O | PRO | A | 128 | 113.424 | 20.517 | 22.373 | 1.00 | 24.57 | O | |
| ATOM | 996 | CB | PRO | A | 128 | 111.338 | 21.611 | 20.225 | 1.00 | 26.25 | C | |
| ATOM | 997 | CG | PRO | A | 128 | 111.184 | 22.717 | 21.333 | 1.00 | 26.88 | C | |
| ATOM | 998 | CD | PRO | A | 128 | 110.192 | 22.095 | 22.247 | 1.00 | 27.23 | C | |
| ATOM | 999 | N | THR | A | 129 | 113.527 | 19.243 | 20.509 | 1.00 | 23.56 | N | |
| ATOM | 1000 | CA | THR | A | 129 | 114.976 | 19.008 | 20.684 | 1.00 | 21.19 | C | |
| ATOM | 1001 | C | THR | A | 129 | 115.655 | 19.300 | 19.324 | 1.00 | 22.02 | C | |
| ATOM | 1002 | O | THR | A | 129 | 115.043 | 19.163 | 18.266 | 1.00 | 21.34 | O | |
| ATOM | 1003 | CB | THR | A | 129 | 115.299 | 17.527 | 21.077 | 1.00 | 21.25 | C | |
| ATOM | 1004 | OG1 | THR | A | 129 | 114.793 | 16.664 | 20.054 | 1.00 | 23.45 | O | |
| ATOM | 1005 | CG2 | THR | A | 129 | 114.651 | 17.181 | 22.452 | 1.00 | 23.71 | C | |
| ATOM | 1006 | N | ILE | A | 130 | 116.900 | 19.747 | 19.363 | 1.00 | 22.22 | N | |
| ATOM | 1007 | CA | ILE | A | 130 | 117.663 | 19.995 | 18.146 | 1.00 | 21.56 | C | |
| ATOM | 1008 | C | ILE | A | 130 | 119.087 | 19.567 | 18.428 | 1.00 | 26.53 | C | |
| ATOM | 1009 | O | ILE | A | 130 | 119.727 | 20.082 | 19.376 | 1.00 | 25.09 | O | |
| ATOM | 1010 | CB | ILE | A | 130 | 117.734 | 21.478 | 17.739 | 1.00 | 26.86 | C | |
| ATOM | 1011 | CG1 | ILE | A | 130 | 116.335 | 22.074 | 17.647 | 1.00 | 29.89 | C | |
| ATOM | 1012 | CG2 | ILE | A | 130 | 118.444 | 21.578 | 16.357 | 1.00 | 25.45 | C | |
| ATOM | 1013 | CD1 | ILE | A | 130 | 116.328 | 23.610 | 17.535 | 1.00 | 31.25 | C | |
| ATOM | 1014 | N | THR | A | 131 | 119.596 | 18.630 | 17.636 | 1.00 | 24.43 | N | |
| ATOM | 1015 | CA | THR | A | 131 | 120.977 | 18.174 | 17.855 | 1.00 | 26.97 | C | |
| ATOM | 1016 | C | THR | A | 131 | 121.810 | 18.292 | 16.575 | 1.00 | 26.32 | C | |
| ATOM | 1017 | O | THR | A | 131 | 121.272 | 18.190 | 15.457 | 1.00 | 25.94 | O | |
| ATOM | 1018 | CB | THR | A | 131 | 121.022 | 16.702 | 18.414 | 1.00 | 29.09 | C | |
| ATOM | 1019 | OG1 | THR | A | 131 | 120.316 | 15.802 | 17.552 | 1.00 | 29.45 | O | |
| ATOM | 1020 | CG2 | THR | A | 131 | 120.334 | 16.645 | 19.757 | 1.00 | 29.05 | C | |
| ATOM | 1021 | N | CYS | A | 132 | 123.105 | 18.552 | 16.735 | 1.00 | 26.85 | N | |
| ATOM | 1022 | CA | CYS | A | 132 | 123.973 | 18.693 | 15.562 | 1.00 | 27.04 | C | |
| ATOM | 1023 | C | CYS | A | 132 | 124.628 | 17.334 | 15.316 | 1.00 | 27.37 | C | |
| ATOM | 1024 | O | CYS | A | 132 | 125.398 | 16.848 | 16.158 | 1.00 | 30.63 | O | |
| ATOM | 1025 | CB | CYS | A | 132 | 125.009 | 19.789 | 15.783 | 1.00 | 25.14 | C | |
| ATOM | 1026 | SG | CYS | A | 132 | 126.188 | 19.890 | 14.369 | 1.00 | 28.62 | S | |
| ATOM | 1027 | N | SER | A | 133 | 124.309 | 16.701 | 14.179 | 1.00 | 26.87 | N | |
| ATOM | 1028 | CA | SER | A | 133 | 124.843 | 15.348 | 13.924 | 1.00 | 30.35 | C | |
| ATOM | 1029 | C | SER | A | 133 | 126.123 | 15.277 | 13.137 | 1.00 | 30.92 | C | |
| ATOM | 1030 | O | SER | A | 133 | 126.722 | 14.206 | 13.021 | 1.00 | 33.53 | O | |
| ATOM | 1031 | CB | SER | A | 133 | 123.793 | 14.455 | 13.273 | 1.00 | 32.04 | C | |
| ATOM | 1032 | OG | SER | A | 133 | 123.232 | 15.057 | 12.126 | 1.00 | 34.97 | O | |
| ATOM | 1033 | N | SER | A | 134 | 126.528 | 16.395 | 12.552 | 1.00 | 29.99 | N | |
| ATOM | 1034 | CA | SER | A | 134 | 127.797 | 16.441 | 11.831 | 1.00 | 29.02 | C | |
| ATOM | 1035 | C | SER | A | 134 | 128.100 | 17.876 | 11.445 | 1.00 | 28.23 | C | |
| ATOM | 1036 | O | SER | A | 134 | 127.205 | 18.732 | 11.394 | 1.00 | 26.16 | O | |
| ATOM | 1037 | CB | SER | A | 134 | 127.813 | 15.563 | 10.565 | 1.00 | 31.49 | C | |
| ATOM | 1038 | OG | SER | A | 134 | 126.858 | 15.959 | 9.620 | 1.00 | 33.50 | O | |
| ATOM | 1039 | N | CYS | A | 135 | 129.375 | 18.125 | 11.188 | 1.00 | 24.39 | N | |
| ATOM | 1040 | CA | CYS | A | 135 | 129.826 | 19.449 | 10.797 | 1.00 | 23.58 | C | |
| ATOM | 1041 | C | CYS | A | 135 | 131.121 | 19.253 | 9.985 | 1.00 | 22.61 | C | |
| ATOM | 1042 | O | CYS | A | 135 | 131.951 | 18.389 | 10.317 | 1.00 | 23.89 | O | |
| ATOM | 1043 | CB | CYS | A | 135 | 130.137 | 20.279 | 12.052 | 1.00 | 22.12 | C | |
| ATOM | 1044 | SG | CYS | A | 135 | 130.678 | 21.987 | 11.760 | 1.00 | 24.00 | S | |
| ATOM | 1045 | N | SER | A | 136 | 131.271 | 20.035 | 8.922 | 1.00 | 21.46 | N | |
| ATOM | 1046 | CA | SER | A | 136 | 132.504 | 20.024 | 8.126 | 1.00 | 22.04 | C | |
| ATOM | 1047 | C | SER | A | 136 | 132.815 | 21.501 | 7.819 | 1.00 | 23.24 | C | |
| ATOM | 1048 | O | SER | A | 136 | 131.922 | 22.324 | 7.786 | 1.00 | 23.47 | O | |
| ATOM | 1049 | CB | SER | A | 136 | 132.361 | 19.181 | 6.846 | 1.00 | 27.15 | C | |
| ATOM | 1050 | OG | SER | A | 136 | 131.277 | 19.599 | 6.063 | 1.00 | 35.21 | O | |
| ATOM | 1051 | N | SER | A | 137 | 134.082 | 21.826 | 7.637 | 1.00 | 23.28 | N | |
| ATOM | 1052 | CA | SER | A | 137 | 134.519 | 23.203 | 7.357 | 1.00 | 21.12 | C | |
| ATOM | 1053 | C | SER | A | 137 | 135.714 | 23.006 | 6.399 | 1.00 | 24.00 | C | |
| ATOM | 1054 | O | SER | A | 137 | 136.613 | 22.184 | 6.676 | 1.00 | 26.28 | O | |
| ATOM | 1055 | CB | SER | A | 137 | 135.008 | 23.871 | 8.664 | 1.00 | 24.86 | C | |
| ATOM | 1056 | OG | SER | A | 137 | 135.592 | 25.171 | 8.466 | 1.00 | 21.45 | O | |
| ATOM | 1057 | N | HIS | A | 138 | 135.720 | 23.718 | 5.274 | 1.00 | 22.89 | N | |
| ATOM | 1058 | CA | HIS | A | 138 | 136.817 | 23.603 | 4.321 | 1.00 | 22.59 | C | |
| ATOM | 1059 | C | HIS | A | 138 | 137.270 | 25.033 | 3.960 | 1.00 | 21.69 | C | |
| ATOM | 1060 | O | HIS | A | 138 | 136.487 | 25.986 | 4.108 | 1.00 | 22.19 | O | |
| ATOM | 1061 | CB | HIS | A | 138 | 136.351 | 22.865 | 3.049 | 1.00 | 24.47 | C | |
| ATOM | 1062 | CG | HIS | A | 138 | 136.005 | 21.429 | 3.270 | 1.00 | 31.02 | C | |
| ATOM | 1063 | ND1 | HIS | A | 138 | 136.937 | 20.414 | 3.162 | 1.00 | 34.51 | N | |
| ATOM | 1064 | CD2 | HIS | A | 138 | 134.829 | 20.831 | 3.575 | 1.00 | 30.58 | C | |
| ATOM | 1065 | CE1 | HIS | A | 138 | 136.345 | 19.254 | 3.391 | 1.00 | 31.58 | C | |
| ATOM | 1066 | NE2 | HIS | A | 138 | 135.066 | 19.483 | 3.645 | 1.00 | 35.06 | N | |
| ATOM | 1067 | N | ILE | A | 139 | 138.506 | 25.177 | 3.480 | 1.00 | 22.03 | N | |
| ATOM | 1068 | CA | ILE | A | 139 | 139.056 | 26.499 | 3.125 | 1.00 | 21.48 | C | |
| ATOM | 1069 | C | ILE | A | 139 | 139.622 | 26.443 | 1.724 | 1.00 | 23.05 | C | |
| ATOM | 1070 | O | ILE | A | 139 | 140.310 | 25.489 | 1.392 | 1.00 | 25.15 | O | |
| ATOM | 1071 | CB | ILE | A | 139 | 140.197 | 26.902 | 4.094 | 1.00 | 24.43 | C | |
| ATOM | 1072 | CG1 | ILE | A | 139 | 139.618 | 27.212 | 5.473 | 1.00 | 23.14 | C | |
| ATOM | 1073 | CG2 | ILE | A | 139 | 141.004 | 28.091 | 3.523 | 1.00 | 21.83 | C | |
| ATOM | 1074 | CD1 | ILE | A | 139 | 140.726 | 27.415 | 6.584 | 1.00 | 23.17 | C | |
| ATOM | 1075 | N | ASN | A | 140 | 139.350 | 27.466 | 0.909 | 1.00 | 24.38 | N | |
| ATOM | 1076 | CA | ASN | A | 140 | 139.867 | 27.433 | −0.464 | 1.00 | 25.84 | C | |
| ATOM | 1077 | C | ASN | A | 140 | 141.297 | 27.943 | −0.609 | 1.00 | 26.82 | C | |
| ATOM | 1078 | O | ASN | A | 140 | 142.129 | 27.306 | −1.280 | 1.00 | 26.46 | O | |
| ATOM | 1079 | CB | ASN | A | 140 | 138.923 | 28.187 | −1.409 | 1.00 | 29.67 | C | |
| ATOM | 1080 | CG | ASN | A | 140 | 139.438 | 28.203 | −2.838 | 1.00 | 33.79 | C | |
| ATOM | 1081 | OD1 | ASN | A | 140 | 139.886 | 29.236 | −3.343 | 1.00 | 38.11 | O | |
| ATOM | 1082 | ND2 | ASN | A | 140 | 139.411 | 27.044 | −3.478 | 1.00 | 29.13 | N | |
| ATOM | 1083 | N | SER | A | 141 | 141.592 | 29.077 | 0.028 | 1.00 | 24.19 | N | |
| ATOM | 1084 | CA | SER | A | 141 | 142.943 | 29.620 | −0.069 | 1.00 | 21.42 | C | |
| ATOM | 1085 | C | SER | A | 141 | 143.304 | 30.511 | 1.115 | 1.00 | 21.81 | C | |
| ATOM | 1086 | O | SER | A | 141 | 142.428 | 30.958 | 1.877 | 1.00 | 20.83 | O | |
| ATOM | 1087 | CB | SER | A | 141 | 143.095 | 30.385 | −1.383 | 1.00 | 25.08 | C | |
| ATOM | 1088 | OG | SER | A | 141 | 142.394 | 31.609 | −1.314 | 1.00 | 24.68 | O | |
| ATOM | 1089 | N | VAL | A | 142 | 144.616 | 30.687 | 1.319 | 1.00 | 20.93 | N | |
| ATOM | 1090 | CA | VAL | A | 142 | 145.137 | 31.530 | 2.413 | 1.00 | 22.71 | C | |
| ATOM | 1091 | C | VAL | A | 142 | 146.206 | 32.455 | 1.855 | 1.00 | 24.50 | C | |
| ATOM | 1092 | O | VAL | A | 142 | 147.129 | 31.983 | 1.148 | 1.00 | 27.06 | O | |
| ATOM | 1093 | CB | VAL | A | 142 | 145.815 | 30.654 | 3.524 | 1.00 | 22.95 | C | |
| ATOM | 1094 | CG1 | VAL | A | 142 | 146.366 | 31.536 | 4.632 | 1.00 | 26.66 | C | |
| ATOM | 1095 | CG2 | VAL | A | 142 | 144.791 | 29.644 | 4.075 | 1.00 | 23.86 | C | |
| ATOM | 1096 | N | AHIS | A | 143 | 146.113 | 33.741 | 2.183 | 0.50 | 22.08 | N | |
| ATOM | 1097 | N | BHIS | A | 143 | 146.113 | 33.750 | 2.158 | 0.50 | 22.98 | N | |
| ATOM | 1098 | CA | AHIS | A | 143 | 147.083 | 34.704 | 1.690 | 0.50 | 23.19 | C | |
| ATOM | 1099 | CA | BHIS | A | 143 | 147.112 | 34.707 | 1.682 | 0.50 | 24.45 | C | |
| ATOM | 1100 | C | AHIS | A | 143 | 147.674 | 35.551 | 2.832 | 0.50 | 22.83 | C | |
| ATOM | 1101 | C | BHIS | A | 143 | 147.683 | 35.509 | 2.852 | 0.50 | 23.68 | C | |
| ATOM | 1102 | O | AHIS | A | 143 | 146.933 | 36.116 | 3.653 | 0.50 | 22.06 | O | |
| ATOM | 1103 | O | BHIS | A | 143 | 146.939 | 35.990 | 3.718 | 0.50 | 23.22 | O | |
| ATOM | 1104 | CB | AHIS | A | 143 | 146.392 | 35.598 | 0.664 | 0.50 | 20.65 | C | |
| ATOM | 1105 | CB | BHIS | A | 143 | 146.483 | 35.670 | 0.668 | 0.50 | 23.70 | C | |
| ATOM | 1106 | CG | AHIS | A | 143 | 145.644 | 34.841 | −0.400 | 0.50 | 22.32 | C | |
| ATOM | 1107 | CG | BHIS | A | 143 | 147.419 | 36.726 | 0.152 | 0.50 | 27.00 | C | |
| ATOM | 1108 | ND1 | AHIS | A | 143 | 146.257 | 34.353 | −1.530 | 0.50 | 25.97 | N | |
| ATOM | 1109 | ND1 | BHIS | A | 143 | 148.351 | 36.481 | −0.830 | 0.50 | 28.37 | N | |
| ATOM | 1110 | CD2 | AHIS | A | 143 | 144.340 | 34.482 | −0.487 | 0.50 | 21.56 | C | |
| ATOM | 1111 | CD2 | BHIS | A | 143 | 147.555 | 38.038 | 0.473 | 0.50 | 27.25 | C | |
| ATOM | 1112 | CE1 | AHIS | A | 143 | 145.360 | 33.726 | −2.276 | 0.50 | 22.09 | C | |
| ATOM | 1113 | CE1 | BHIS | A | 143 | 149.019 | 37.591 | −1.096 | 0.50 | 28.23 | C | |
| ATOM | 1114 | NE2 | AHIS | A | 143 | 144.191 | 33.789 | −1.665 | 0.50 | 24.64 | N | |
| ATOM | 1115 | NE2 | BHIS | A | 143 | 148.557 | 38.551 | −0.317 | 0.50 | 25.58 | N | |
| ATOM | 1116 | N | VAL | A | 144 | 149.006 | 35.658 | 2.855 | 1.00 | 22.26 | N | |
| ATOM | 1117 | CA | VAL | A | 144 | 149.710 | 36.432 | 3.899 | 1.00 | 23.31 | C | |
| ATOM | 1118 | C | VAL | A | 144 | 150.288 | 37.716 | 3.282 | 1.00 | 23.12 | C | |
| ATOM | 1119 | O | VAL | A | 144 | 150.855 | 37.668 | 2.174 | 1.00 | 22.14 | O | |
| ATOM | 1120 | CB | VAL | A | 144 | 150.909 | 35.645 | 4.476 | 1.00 | 25.98 | C | |
| ATOM | 1121 | CG1 | VAL | A | 144 | 151.446 | 36.385 | 5.710 | 1.00 | 25.04 | C | |
| ATOM | 1122 | CG2 | VAL | A | 144 | 150.505 | 34.191 | 4.828 | 1.00 | 30.60 | C | |
| ATOM | 1123 | N | HIS | A | 145 | 150.118 | 38.855 | 3.949 | 1.00 | 20.90 | N | |
| ATOM | 1124 | CA | HIS | A | 145 | 150.690 | 40.117 | 3.453 | 1.00 | 20.43 | C | |
| ATOM | 1125 | C | HIS | A | 145 | 151.527 | 40.727 | 4.563 | 1.00 | 20.71 | C | |
| ATOM | 1126 | O | HIS | A | 145 | 151.091 | 40.792 | 5.713 | 1.00 | 21.06 | O | |
| ATOM | 1127 | CB | HIS | A | 145 | 149.620 | 41.158 | 3.069 | 1.00 | 22.08 | C | |
| ATOM | 1128 | CG | HIS | A | 145 | 150.173 | 42.542 | 2.818 | 1.00 | 26.10 | C | |
| ATOM | 1129 | ND1 | HIS | A | 145 | 150.051 | 43.575 | 3.731 | 1.00 | 28.27 | N | |
| ATOM | 1130 | CD2 | HIS | A | 145 | 150.887 | 43.042 | 1.780 | 1.00 | 27.55 | C | |
| ATOM | 1131 | CE1 | HIS | A | 145 | 150.668 | 44.646 | 3.263 | 1.00 | 25.47 | C | |
| ATOM | 1132 | NE2 | HIS | A | 145 | 151.180 | 44.354 | 2.082 | 1.00 | 28.96 | N | |
| ATOM | 1133 | N | ILE | A | 146 | 152.743 | 41.139 | 4.210 | 1.00 | 18.59 | N | |
| ATOM | 1134 | CA | ILE | A | 146 | 153.609 | 41.835 | 5.167 | 1.00 | 18.37 | C | |
| ATOM | 1135 | C | ILE | A | 146 | 154.094 | 43.107 | 4.467 | 1.00 | 18.36 | C | |
| ATOM | 1136 | O | ILE | A | 146 | 154.538 | 43.055 | 3.338 | 1.00 | 17.91 | O | |
| ATOM | 1137 | CB | ILE | A | 146 | 154.805 | 40.920 | 5.609 | 1.00 | 18.23 | C | |
| ATOM | 1138 | CG1 | ILE | A | 146 | 154.277 | 39.813 | 6.548 | 1.00 | 20.19 | C | |
| ATOM | 1139 | CG2 | ILE | A | 146 | 155.882 | 41.750 | 6.263 | 1.00 | 19.05 | C | |
| ATOM | 1140 | CD1 | ILE | A | 146 | 155.239 | 38.633 | 6.787 | 1.00 | 21.40 | C | |
| ATOM | 1141 | N | SER | A | 147 | 153.973 | 44.247 | 5.144 | 1.00 | 20.80 | N | |
| ATOM | 1142 | CA | SER | A | 147 | 154.378 | 45.529 | 4.554 | 1.00 | 22.81 | C | |
| ATOM | 1143 | C | SER | A | 147 | 155.837 | 45.584 | 4.090 | 1.00 | 23.25 | C | |
| ATOM | 1144 | O | SER | A | 147 | 156.169 | 46.280 | 3.108 | 1.00 | 26.01 | O | |
| ATOM | 1145 | CB | SER | A | 147 | 154.109 | 46.683 | 5.546 | 1.00 | 25.27 | C | |
| ATOM | 1146 | OG | SER | A | 147 | 155.057 | 46.680 | 6.607 | 1.00 | 30.37 | O | |
| ATOM | 1147 | N | LYS | A | 148 | 156.728 | 44.878 | 4.782 | 1.00 | 24.52 | N | |
| ATOM | 1148 | CA | LYS | A | 148 | 158.136 | 44.872 | 4.359 | 1.00 | 25.59 | C | |
| ATOM | 1149 | C | LYS | A | 148 | 158.293 | 43.850 | 3.213 | 1.00 | 25.07 | C | |
| ATOM | 1150 | O | LYS | A | 148 | 158.142 | 42.646 | 3.416 | 1.00 | 24.80 | O | |
| ATOM | 1151 | CB | LYS | A | 148 | 159.083 | 44.456 | 5.489 | 1.00 | 26.95 | C | |
| ATOM | 1152 | CG | LYS | A | 148 | 159.048 | 45.283 | 6.765 | 1.00 | 36.35 | C | |
| ATOM | 1153 | CD | LYS | A | 148 | 160.027 | 44.654 | 7.815 | 1.00 | 37.70 | C | |
| ATOM | 1154 | CE | LYS | A | 148 | 160.108 | 45.463 | 9.108 | 1.00 | 42.14 | C | |
| ATOM | 1155 | NZ | LYS | A | 148 | 158.820 | 45.451 | 9.867 | 1.00 | 44.10 | N | |
| ATOM | 1156 | N | SER | A | 149 | 158.649 | 44.343 | 2.027 | 1.00 | 23.82 | N | |
| ATOM | 1157 | CA | SER | A | 149 | 158.801 | 43.501 | 0.831 | 1.00 | 23.15 | C | |
| ATOM | 1158 | C | SER | A | 149 | 159.932 | 42.495 | 0.900 | 1.00 | 25.21 | C | |
| ATOM | 1159 | O | SER | A | 149 | 159.894 | 41.459 | 0.204 | 1.00 | 24.56 | O | |
| ATOM | 1160 | CB | SER | A | 149 | 159.002 | 44.388 | −0.419 | 1.00 | 27.76 | C | |
| ATOM | 1161 | OG | SER | A | 149 | 160.205 | 45.136 | −0.300 | 1.00 | 22.89 | O | |
| ATOM | 1162 | N | LYS | A | 150 | 160.924 | 42.749 | 1.754 | 1.00 | 22.84 | N | |
| ATOM | 1163 | CA | LYS | A | 150 | 162.064 | 41.825 | 1.789 | 1.00 | 23.22 | C | |
| ATOM | 1164 | C | LYS | A | 150 | 161.823 | 40.439 | 2.389 | 1.00 | 23.06 | C | |
| ATOM | 1165 | O | LYS | A | 150 | 162.769 | 39.622 | 2.339 | 1.00 | 21.71 | O | |
| ATOM | 1166 | CB | LYS | A | 150 | 163.230 | 42.439 | 2.545 | 1.00 | 27.64 | C | |
| ATOM | 1167 | CG | LYS | A | 150 | 162.935 | 42.572 | 4.022 | 1.00 | 31.09 | C | |
| ATOM | 1168 | CD | LYS | A | 150 | 164.136 | 43.113 | 4.840 | 1.00 | 40.99 | C | |
| ATOM | 1169 | CE | LYS | A | 150 | 164.924 | 44.197 | 4.109 | 1.00 | 42.87 | C | |
| ATOM | 1170 | NZ | LYS | A | 150 | 165.938 | 43.605 | 3.192 | 1.00 | 48.81 | N | |
| ATOM | 1171 | N | VAL | A | 151 | 160.618 | 40.146 | 2.907 | 1.00 | 23.33 | N | |
| ATOM | 1172 | CA | VAL | A | 151 | 160.411 | 38.820 | 3.517 | 1.00 | 24.28 | C | |
| ATOM | 1173 | C | VAL | A | 151 | 159.562 | 37.812 | 2.770 | 1.00 | 23.01 | C | |
| ATOM | 1174 | O | VAL | A | 151 | 158.873 | 37.001 | 3.360 | 1.00 | 20.36 | O | |
| ATOM | 1175 | CB | VAL | A | 151 | 159.943 | 38.942 | 5.004 | 1.00 | 28.12 | C | |
| ATOM | 1176 | CG1 | VAL | A | 151 | 161.086 | 39.603 | 5.820 | 1.00 | 26.68 | C | |
| ATOM | 1177 | CG2 | VAL | A | 151 | 158.637 | 39.768 | 5.108 | 1.00 | 28.79 | C | |
| ATOM | 1178 | N | GLY | A | 152 | 159.649 | 37.889 | 1.444 | 1.00 | 21.86 | N | |
| ATOM | 1179 | CA | GLY | A | 152 | 158.959 | 36.964 | 0.571 | 1.00 | 21.76 | C | |
| ATOM | 1180 | C | GLY | A | 152 | 159.370 | 35.553 | 0.948 | 1.00 | 23.30 | C | |
| ATOM | 1181 | O | GLY | A | 152 | 158.542 | 34.644 | 0.915 | 1.00 | 20.77 | O | |
| ATOM | 1182 | N | TRP | A | 153 | 160.639 | 35.369 | 1.333 | 1.00 | 21.70 | N | |
| ATOM | 1183 | CA | TRP | A | 153 | 161.115 | 34.036 | 1.707 | 1.00 | 21.21 | C | |
| ATOM | 1184 | C | TRP | A | 153 | 160.382 | 33.517 | 2.951 | 1.00 | 19.05 | C | |
| ATOM | 1185 | O | TRP | A | 153 | 160.125 | 32.324 | 3.073 | 1.00 | 21.95 | O | |
| ATOM | 1186 | CB | TRP | A | 153 | 162.659 | 34.031 | 1.983 | 1.00 | 19.77 | C | |
| ATOM | 1187 | CG | TRP | A | 153 | 163.110 | 34.959 | 3.117 | 1.00 | 21.64 | C | |
| ATOM | 1188 | CD1 | TRP | A | 153 | 163.430 | 36.287 | 3.014 | 1.00 | 22.16 | C | |
| ATOM | 1189 | CD2 | TRP | A | 153 | 163.195 | 34.633 | 4.520 | 1.00 | 23.76 | C | |
| ATOM | 1190 | NE1 | TRP | A | 153 | 163.709 | 36.808 | 4.253 | 1.00 | 21.11 | N | |
| ATOM | 1191 | CE2 | TRP | A | 153 | 163.568 | 35.823 | 5.203 | 1.00 | 22.73 | C | |
| ATOM | 1192 | CE3 | TRP | A | 153 | 162.996 | 33.451 | 5.264 | 1.00 | 23.26 | C | |
| ATOM | 1193 | CZ2 | TRP | A | 153 | 163.749 | 35.869 | 6.614 | 1.00 | 27.22 | C | |
| ATOM | 1194 | CZ3 | TRP | A | 153 | 163.181 | 33.489 | 6.668 | 1.00 | 25.75 | C | |
| ATOM | 1195 | CH2 | TRP | A | 153 | 163.552 | 34.698 | 7.323 | 1.00 | 28.15 | C | |
| ATOM | 1196 | N | LEU | A | 154 | 160.050 | 34.406 | 3.872 | 1.00 | 17.94 | N | |
| ATOM | 1197 | CA | LEU | A | 154 | 159.369 | 33.986 | 5.108 | 1.00 | 20.50 | C | |
| ATOM | 1198 | C | LEU | A | 154 | 157.921 | 33.613 | 4.782 | 1.00 | 21.27 | C | |
| ATOM | 1199 | O | LEU | A | 154 | 157.374 | 32.668 | 5.353 | 1.00 | 22.45 | O | |
| ATOM | 1200 | CB | LEU | A | 154 | 159.425 | 35.098 | 6.153 | 1.00 | 19.41 | C | |
| ATOM | 1201 | CG | LEU | A | 154 | 158.700 | 34.896 | 7.489 | 1.00 | 22.12 | C | |
| ATOM | 1202 | CD1 | LEU | A | 154 | 159.320 | 33.681 | 8.230 | 1.00 | 21.73 | C | |
| ATOM | 1203 | CD2 | LEU | A | 154 | 158.839 | 36.171 | 8.342 | 1.00 | 23.69 | C | |
| ATOM | 1204 | N | ILE | A | 155 | 157.288 | 34.345 | 3.863 | 1.00 | 18.31 | N | |
| ATOM | 1205 | CA | ILE | A | 155 | 155.909 | 33.987 | 3.519 | 1.00 | 19.89 | C | |
| ATOM | 1206 | C | ILE | A | 155 | 155.915 | 32.611 | 2.812 | 1.00 | 18.64 | C | |
| ATOM | 1207 | O | ILE | A | 155 | 154.995 | 31.814 | 2.980 | 1.00 | 22.17 | O | |
| ATOM | 1208 | CB | ILE | A | 155 | 155.287 | 35.097 | 2.639 | 1.00 | 20.74 | C | |
| ATOM | 1209 | CG1 | ILE | A | 155 | 155.005 | 36.321 | 3.521 | 1.00 | 25.44 | C | |
| ATOM | 1210 | CG2 | ILE | A | 155 | 154.012 | 34.579 | 1.975 | 1.00 | 21.41 | C | |
| ATOM | 1211 | CD1 | ILE | A | 155 | 154.543 | 37.635 | 2.785 | 1.00 | 20.68 | C | |
| ATOM | 1212 | N | GLN | A | 156 | 156.957 | 32.335 | 2.034 | 1.00 | 18.94 | N | |
| ATOM | 1213 | CA | GLN | A | 156 | 157.078 | 31.024 | 1.396 | 1.00 | 18.98 | C | |
| ATOM | 1214 | C | GLN | A | 156 | 157.252 | 29.940 | 2.485 | 1.00 | 18.90 | C | |
| ATOM | 1215 | O | GLN | A | 156 | 156.670 | 28.834 | 2.373 | 1.00 | 21.67 | O | |
| ATOM | 1216 | CB | GLN | A | 156 | 158.271 | 30.981 | 0.424 | 1.00 | 19.59 | C | |
| ATOM | 1217 | CG | GLN | A | 156 | 158.407 | 29.626 | −0.259 | 1.00 | 25.02 | C | |
| ATOM | 1218 | CD | GLN | A | 156 | 157.287 | 29.292 | −1.249 | 1.00 | 28.00 | C | |
| ATOM | 1219 | OE1 | GLN | A | 156 | 156.146 | 29.766 | −1.142 | 1.00 | 30.44 | O | |
| ATOM | 1220 | NE2 | GLN | A | 156 | 157.622 | 28.435 | −2.238 | 1.00 | 33.99 | N | |
| ATOM | 1221 | N | LEU | A | 157 | 158.013 | 30.220 | 3.551 | 1.00 | 20.68 | N | |
| ATOM | 1222 | CA | LEU | A | 157 | 158.125 | 29.189 | 4.604 | 1.00 | 21.82 | C | |
| ATOM | 1223 | C | LEU | A | 157 | 156.748 | 28.964 | 5.251 | 1.00 | 22.51 | C | |
| ATOM | 1224 | O | LEU | A | 157 | 156.389 | 27.839 | 5.596 | 1.00 | 23.51 | O | |
| ATOM | 1225 | CB | LEU | A | 157 | 159.154 | 29.612 | 5.684 | 1.00 | 23.40 | C | |
| ATOM | 1226 | CG | LEU | A | 157 | 160.607 | 29.558 | 5.186 | 1.00 | 25.25 | C | |
| ATOM | 1227 | CD1 | LEU | A | 157 | 161.554 | 30.120 | 6.251 | 1.00 | 28.48 | C | |
| ATOM | 1228 | CD2 | LEU | A | 157 | 160.978 | 28.086 | 4.845 | 1.00 | 32.84 | C | |
| ATOM | 1229 | N | PHE | A | 158 | 155.992 | 30.046 | 5.424 | 1.00 | 21.43 | N | |
| ATOM | 1230 | CA | PHE | A | 158 | 154.647 | 29.931 | 6.000 | 1.00 | 22.87 | C | |
| ATOM | 1231 | C | PHE | A | 158 | 153.801 | 28.953 | 5.178 | 1.00 | 22.22 | C | |
| ATOM | 1232 | O | PHE | A | 158 | 153.173 | 28.013 | 5.732 | 1.00 | 21.69 | O | |
| ATOM | 1233 | CB | PHE | A | 158 | 153.960 | 31.309 | 6.102 | 1.00 | 20.44 | C | |
| ATOM | 1234 | CG | PHE | A | 158 | 152.504 | 31.212 | 6.441 | 1.00 | 22.66 | C | |
| ATOM | 1235 | CD1 | PHE | A | 158 | 151.562 | 30.910 | 5.462 | 1.00 | 23.07 | C | |
| ATOM | 1236 | CD2 | PHE | A | 158 | 152.079 | 31.403 | 7.739 | 1.00 | 23.30 | C | |
| ATOM | 1237 | CE1 | PHE | A | 158 | 150.197 | 30.804 | 5.781 | 1.00 | 22.87 | C | |
| ATOM | 1238 | CE2 | PHE | A | 158 | 150.731 | 31.300 | 8.085 | 1.00 | 24.31 | C | |
| ATOM | 1239 | CZ | PHE | A | 158 | 149.775 | 31.001 | 7.108 | 1.00 | 22.21 | C | |
| ATOM | 1240 | N | HIS | A | 159 | 153.778 | 29.165 | 3.858 | 1.00 | 22.94 | N | |
| ATOM | 1241 | CA | HIS | A | 159 | 153.033 | 28.262 | 2.981 | 1.00 | 23.08 | C | |
| ATOM | 1242 | C | HIS | A | 159 | 153.550 | 26.813 | 3.029 | 1.00 | 24.35 | C | |
| ATOM | 1243 | O | HIS | A | 159 | 152.769 | 25.871 | 3.125 | 1.00 | 23.24 | O | |
| ATOM | 1244 | CB | HIS | A | 159 | 153.074 | 28.785 | 1.541 | 1.00 | 24.96 | C | |
| ATOM | 1245 | CG | HIS | A | 159 | 152.237 | 30.005 | 1.338 | 1.00 | 24.81 | C | |
| ATOM | 1246 | ND1 | HIS | A | 159 | 150.861 | 29.973 | 1.446 | 1.00 | 28.89 | N | |
| ATOM | 1247 | CD2 | HIS | A | 159 | 152.563 | 31.280 | 1.030 | 1.00 | 27.35 | C | |
| ATOM | 1248 | CE1 | HIS | A | 159 | 150.379 | 31.182 | 1.216 | 1.00 | 25.70 | C | |
| ATOM | 1249 | NE2 | HIS | A | 159 | 151.393 | 31.994 | 0.959 | 1.00 | 24.73 | N | |
| ATOM | 1250 | N | LYS | A | 160 | 154.867 | 26.635 | 2.974 | 1.00 | 25.32 | N | |
| ATOM | 1251 | CA | LYS | A | 160 | 155.416 | 25.285 | 3.010 | 1.00 | 28.19 | C | |
| ATOM | 1252 | C | LYS | A | 160 | 155.332 | 24.548 | 4.337 | 1.00 | 26.65 | C | |
| ATOM | 1253 | O | LYS | A | 160 | 155.135 | 23.330 | 4.330 | 1.00 | 27.81 | O | |
| ATOM | 1254 | CB | LYS | A | 160 | 156.875 | 25.272 | 2.567 | 1.00 | 30.97 | C | |
| ATOM | 1255 | CG | LYS | A | 160 | 157.036 | 25.240 | 1.061 | 1.00 | 37.66 | C | |
| ATOM | 1256 | CD | LYS | A | 160 | 158.491 | 24.947 | 0.659 | 1.00 | 40.59 | C | |
| ATOM | 1257 | CE | LYS | A | 160 | 159.484 | 25.814 | 1.456 | 1.00 | 41.81 | C | |
| ATOM | 1258 | NZ | LYS | A | 160 | 160.920 | 25.547 | 1.105 | 1.00 | 39.99 | N | |
| ATOM | 1259 | N | LYS | A | 161 | 155.479 | 25.255 | 5.456 | 1.00 | 23.14 | N | |
| ATOM | 1260 | CA | LYS | A | 161 | 155.491 | 24.592 | 6.756 | 1.00 | 27.05 | C | |
| ATOM | 1261 | C | LYS | A | 161 | 154.407 | 24.870 | 7.803 | 1.00 | 26.50 | C | |
| ATOM | 1262 | O | LYS | A | 161 | 154.272 | 24.107 | 8.760 | 1.00 | 26.75 | O | |
| ATOM | 1263 | CB | LYS | A | 161 | 156.852 | 24.824 | 7.415 | 1.00 | 30.17 | C | |
| ATOM | 1264 | CG | LYS | A | 161 | 158.025 | 24.599 | 6.460 | 1.00 | 30.70 | C | |
| ATOM | 1265 | CD | LYS | A | 161 | 159.303 | 25.205 | 7.024 | 1.00 | 42.22 | C | |
| ATOM | 1266 | CE | LYS | A | 161 | 159.669 | 24.604 | 8.393 | 1.00 | 43.48 | C | |
| ATOM | 1267 | NZ | LYS | A | 161 | 160.121 | 23.170 | 8.305 | 1.00 | 50.98 | N | |
| ATOM | 1268 | N | ILE | A | 162 | 153.625 | 25.934 | 7.634 | 1.00 | 24.98 | N | |
| ATOM | 1269 | CA | ILE | A | 162 | 152.595 | 26.289 | 8.591 | 1.00 | 26.09 | C | |
| ATOM | 1270 | C | ILE | A | 162 | 151.152 | 26.225 | 8.048 | 1.00 | 24.13 | C | |
| ATOM | 1271 | O | ILE | A | 162 | 150.242 | 25.762 | 8.739 | 1.00 | 22.91 | O | |
| ATOM | 1272 | CB | ILE | A | 162 | 152.867 | 27.732 | 9.135 | 1.00 | 20.66 | C | |
| ATOM | 1273 | CG1 | ILE | A | 162 | 154.238 | 27.762 | 9.829 | 1.00 | 23.98 | C | |
| ATOM | 1274 | CG2 | ILE | A | 162 | 151.697 | 28.231 | 10.048 | 1.00 | 22.51 | C | |
| ATOM | 1275 | CD1 | ILE | A | 162 | 154.655 | 29.111 | 10.281 | 1.00 | 25.86 | C | |
| ATOM | 1276 | N | GLU | A | 163 | 150.943 | 26.664 | 6.813 | 1.00 | 23.29 | N | |
| ATOM | 1277 | CA | GLU | A | 163 | 149.587 | 26.738 | 6.251 | 1.00 | 23.82 | C | |
| ATOM | 1278 | C | GLU | A | 163 | 148.663 | 25.542 | 6.477 | 1.00 | 25.26 | C | |
| ATOM | 1279 | O | GLU | A | 163 | 147.525 | 25.723 | 6.916 | 1.00 | 25.68 | O | |
| ATOM | 1280 | CB | GLU | A | 163 | 149.625 | 27.050 | 4.729 | 1.00 | 23.25 | C | |
| ATOM | 1281 | CG | GLU | A | 163 | 148.245 | 27.344 | 4.141 | 1.00 | 23.18 | C | |
| ATOM | 1282 | CD | GLU | A | 163 | 148.283 | 27.473 | 2.635 | 1.00 | 27.42 | C | |
| ATOM | 1283 | OE1 | GLU | A | 163 | 149.367 | 27.757 | 2.111 | 1.00 | 26.38 | O | |
| ATOM | 1284 | OE2 | GLU | A | 163 | 147.231 | 27.297 | 1.979 | 1.00 | 33.94 | O | |
| ATOM | 1285 | N | SER | A | 164 | 149.115 | 24.330 | 6.157 | 1.00 | 24.25 | N | |
| ATOM | 1286 | CA | SER | A | 164 | 148.265 | 23.163 | 6.352 | 1.00 | 26.69 | C | |
| ATOM | 1287 | C | SER | A | 164 | 147.808 | 23.004 | 7.793 | 1.00 | 25.60 | C | |
| ATOM | 1288 | O | SER | A | 164 | 146.629 | 22.766 | 8.045 | 1.00 | 25.68 | O | |
| ATOM | 1289 | CB | SER | A | 164 | 148.992 | 21.897 | 5.894 | 1.00 | 29.87 | C | |
| ATOM | 1290 | OG | SER | A | 164 | 149.123 | 21.922 | 4.491 | 1.00 | 38.03 | O | |
| ATOM | 1291 | N | ALA | A | 165 | 148.749 | 23.089 | 8.735 | 1.00 | 26.04 | N | |
| ATOM | 1292 | CA | ALA | A | 165 | 148.429 | 22.975 | 10.146 | 1.00 | 24.77 | C | |
| ATOM | 1293 | C | ALA | A | 165 | 147.417 | 24.043 | 10.584 | 1.00 | 22.29 | C | |
| ATOM | 1294 | O | ALA | A | 165 | 146.432 | 23.767 | 11.317 | 1.00 | 24.60 | O | |
| ATOM | 1295 | CB | ALA | A | 165 | 149.743 | 23.106 | 10.968 | 1.00 | 23.75 | C | |
| ATOM | 1296 | N | LEU | A | 166 | 147.681 | 25.280 | 10.165 | 1.00 | 23.43 | N | |
| ATOM | 1297 | CA | LEU | A | 166 | 146.852 | 26.381 | 10.522 | 1.00 | 21.35 | C | |
| ATOM | 1298 | C | LEU | A | 166 | 145.437 | 26.170 | 9.934 | 1.00 | 23.20 | C | |
| ATOM | 1299 | O | LEU | A | 166 | 144.447 | 26.411 | 10.625 | 1.00 | 24.09 | O | |
| ATOM | 1300 | CB | LEU | A | 166 | 147.501 | 27.680 | 10.037 | 1.00 | 23.96 | C | |
| ATOM | 1301 | CG | LEU | A | 166 | 146.830 | 28.957 | 10.524 | 1.00 | 22.68 | C | |
| ATOM | 1302 | CD1 | LEU | A | 166 | 147.852 | 30.159 | 10.553 | 1.00 | 22.46 | C | |
| ATOM | 1303 | CD2 | LEU | A | 166 | 145.662 | 29.262 | 9.568 | 1.00 | 28.35 | C | |
| ATOM | 1304 | N | ARG | A | 167 | 145.350 | 25.688 | 8.692 | 1.00 | 22.59 | N | |
| ATOM | 1305 | CA | ARG | A | 167 | 144.029 | 25.447 | 8.095 | 1.00 | 24.48 | C | |
| ATOM | 1306 | C | ARG | A | 167 | 143.263 | 24.343 | 8.836 | 1.00 | 25.83 | C | |
| ATOM | 1307 | O | ARG | A | 167 | 142.080 | 24.509 | 9.139 | 1.00 | 24.96 | O | |
| ATOM | 1308 | CB | ARG | A | 167 | 144.134 | 25.008 | 6.640 | 1.00 | 24.06 | C | |
| ATOM | 1309 | CG | ARG | A | 167 | 144.655 | 26.017 | 5.634 | 1.00 | 28.13 | C | |
| ATOM | 1310 | CD | ARG | A | 167 | 144.776 | 25.227 | 4.310 | 1.00 | 34.69 | C | |
| ATOM | 1311 | NE | ARG | A | 167 | 144.614 | 26.019 | 3.101 | 1.00 | 41.99 | N | |
| ATOM | 1312 | CZ | ARG | A | 167 | 143.899 | 25.649 | 2.041 | 1.00 | 42.97 | C | |
| ATOM | 1313 | NH1 | ARG | A | 167 | 143.253 | 24.490 | 2.022 | 1.00 | 41.33 | N | |
| ATOM | 1314 | NH2 | ARG | A | 167 | 143.836 | 26.445 | 0.985 | 1.00 | 45.06 | N | |
| ATOM | 1315 | N | ASN | A | 168 | 143.942 | 23.232 | 9.117 | 1.00 | 26.42 | N | |
| ATOM | 1316 | CA | ASN | A | 168 | 143.322 | 22.113 | 9.822 | 1.00 | 26.35 | C | |
| ATOM | 1317 | C | ASN | A | 168 | 142.766 | 22.530 | 11.167 | 1.00 | 26.11 | C | |
| ATOM | 1318 | O | ASN | A | 168 | 141.610 | 22.182 | 11.559 | 1.00 | 23.82 | O | |
| ATOM | 1319 | CB | ASN | A | 168 | 144.352 | 20.990 | 10.012 | 1.00 | 27.98 | C | |
| ATOM | 1320 | CG | ASN | A | 168 | 144.629 | 20.255 | 8.727 | 1.00 | 35.00 | C | |
| ATOM | 1321 | OD1 | ASN | A | 168 | 143.873 | 20.387 | 7.765 | 1.00 | 37.41 | O | |
| ATOM | 1322 | ND2 | ASN | A | 168 | 145.694 | 19.461 | 8.699 | 1.00 | 36.19 | N | |
| ATOM | 1323 | N | LYS | A | 169 | 143.570 | 23.302 | 11.887 | 1.00 | 23.11 | N | |
| ATOM | 1324 | CA | LYS | A | 169 | 143.155 | 23.765 | 13.205 | 1.00 | 25.98 | C | |
| ATOM | 1325 | C | LYS | A | 169 | 141.978 | 24.734 | 13.119 | 1.00 | 22.95 | C | |
| ATOM | 1326 | O | LYS | A | 169 | 141.048 | 24.670 | 13.925 | 1.00 | 27.85 | O | |
| ATOM | 1327 | CB | LYS | A | 169 | 144.350 | 24.417 | 13.930 | 1.00 | 22.85 | C | |
| ATOM | 1328 | CG | LYS | A | 169 | 144.043 | 24.897 | 15.340 | 1.00 | 29.27 | C | |
| ATOM | 1329 | CD | LYS | A | 169 | 143.707 | 23.731 | 16.267 | 1.00 | 27.60 | C | |
| ATOM | 1330 | CE | LYS | A | 169 | 143.555 | 24.187 | 17.700 | 1.00 | 33.35 | C | |
| ATOM | 1331 | NZ | LYS | A | 169 | 143.216 | 22.995 | 18.562 | 1.00 | 37.61 | N | |
| ATOM | 1332 | N | MET | A | 170 | 142.011 | 25.640 | 12.145 | 1.00 | 22.68 | N | |
| ATOM | 1333 | CA | MET | A | 170 | 140.929 | 26.603 | 12.007 | 1.00 | 24.64 | C | |
| ATOM | 1334 | C | MET | A | 170 | 139.621 | 25.862 | 11.706 | 1.00 | 20.15 | C | |
| ATOM | 1335 | O | MET | A | 170 | 138.581 | 26.187 | 12.303 | 1.00 | 23.65 | O | |
| ATOM | 1336 | CB | MET | A | 170 | 141.233 | 27.592 | 10.887 | 1.00 | 26.04 | C | |
| ATOM | 1337 | CG | MET | A | 170 | 140.196 | 28.719 | 10.741 | 1.00 | 29.87 | C | |
| ATOM | 1338 | SD | MET | A | 170 | 140.037 | 29.809 | 12.216 | 1.00 | 35.30 | S | |
| ATOM | 1339 | CE | MET | A | 170 | 141.411 | 30.729 | 12.064 | 1.00 | 30.78 | C | |
| ATOM | 1340 | N | ASN | A | 171 | 139.669 | 24.891 | 10.801 | 1.00 | 22.71 | N | |
| ATOM | 1341 | CA | ASN | A | 171 | 138.456 | 24.129 | 10.447 | 1.00 | 24.07 | C | |
| ATOM | 1342 | C | ASN | A | 171 | 137.913 | 23.395 | 11.657 | 1.00 | 25.60 | C | |
| ATOM | 1343 | O | ASN | A | 171 | 136.689 | 23.354 | 11.864 | 1.00 | 23.50 | O | |
| ATOM | 1344 | CB | ASN | A | 171 | 138.708 | 23.152 | 9.280 | 1.00 | 23.76 | C | |
| ATOM | 1345 | CG | ASN | A | 171 | 138.843 | 23.897 | 7.905 | 1.00 | 23.11 | C | |
| ATOM | 1346 | OD1 | ASN | A | 171 | 138.101 | 24.820 | 7.620 | 1.00 | 25.38 | O | |
| ATOM | 1347 | ND2 | ASN | A | 171 | 139.804 | 23.476 | 7.077 | 1.00 | 26.17 | N | |
| ATOM | 1348 | N | SER | A | 172 | 138.800 | 22.828 | 12.490 | 1.00 | 24.48 | N | |
| ATOM | 1349 | CA | SER | A | 172 | 138.298 | 22.142 | 13.698 | 1.00 | 26.82 | C | |
| ATOM | 1350 | C | SER | A | 172 | 137.740 | 23.147 | 14.732 | 1.00 | 22.82 | C | |
| ATOM | 1351 | O | SER | A | 172 | 136.695 | 22.911 | 15.372 | 1.00 | 25.07 | O | |
| ATOM | 1352 | CB | SER | A | 172 | 139.399 | 21.258 | 14.324 | 1.00 | 30.57 | C | |
| ATOM | 1353 | OG | SER | A | 172 | 140.439 | 22.073 | 14.824 | 1.00 | 39.62 | O | |
| ATOM | 1354 | N | GLN | A | 173 | 138.406 | 24.277 | 14.919 | 1.00 | 23.86 | N | |
| ATOM | 1355 | CA | GLN | A | 173 | 137.864 | 25.239 | 15.863 | 1.00 | 24.83 | C | |
| ATOM | 1356 | C | GLN | A | 173 | 136.505 | 25.763 | 15.362 | 1.00 | 25.26 | C | |
| ATOM | 1357 | O | GLN | A | 173 | 135.560 | 25.981 | 16.150 | 1.00 | 27.23 | O | |
| ATOM | 1358 | CB | GLN | A | 173 | 138.864 | 26.372 | 16.065 | 1.00 | 29.45 | C | |
| ATOM | 1359 | CG | GLN | A | 173 | 140.103 | 25.912 | 16.774 | 1.00 | 30.82 | C | |
| ATOM | 1360 | CD | GLN | A | 173 | 140.996 | 27.092 | 17.218 | 1.00 | 37.51 | C | |
| ATOM | 1361 | OE1 | GLN | A | 173 | 141.633 | 27.769 | 16.400 | 1.00 | 44.33 | O | |
| ATOM | 1362 | NE2 | GLN | A | 173 | 141.010 | 27.352 | 18.509 | 1.00 | 39.89 | N | |
| ATOM | 1363 | N | VAL | A | 174 | 136.387 | 25.964 | 14.058 | 1.00 | 25.13 | N | |
| ATOM | 1364 | CA | VAL | A | 174 | 135.106 | 26.432 | 13.534 | 1.00 | 24.65 | C | |
| ATOM | 1365 | C | VAL | A | 174 | 133.994 | 25.407 | 13.791 | 1.00 | 21.41 | C | |
| ATOM | 1366 | O | VAL | A | 174 | 132.947 | 25.770 | 14.289 | 1.00 | 24.63 | O | |
| ATOM | 1367 | CB | VAL | A | 174 | 135.184 | 26.750 | 12.023 | 1.00 | 25.38 | C | |
| ATOM | 1368 | CG1 | VAL | A | 174 | 133.754 | 26.834 | 11.420 | 1.00 | 27.61 | C | |
| ATOM | 1369 | CG2 | VAL | A | 174 | 135.911 | 28.068 | 11.826 | 1.00 | 24.79 | C | |
| ATOM | 1370 | N | CYS | A | 175 | 134.210 | 24.131 | 13.513 | 1.00 | 21.16 | N | |
| ATOM | 1371 | CA | CYS | A | 175 | 133.135 | 23.157 | 13.780 | 1.00 | 24.75 | C | |
| ATOM | 1372 | C | CYS | A | 175 | 132.847 | 22.940 | 15.285 | 1.00 | 26.89 | C | |
| ATOM | 1373 | O | CYS | A | 175 | 131.742 | 22.558 | 15.702 | 1.00 | 24.35 | O | |
| ATOM | 1374 | CB | CYS | A | 175 | 133.444 | 21.857 | 13.069 | 1.00 | 24.54 | C | |
| ATOM | 1375 | SG | CYS | A | 175 | 132.689 | 21.829 | 11.399 | 1.00 | 24.95 | S | |
| ATOM | 1376 | N | GLU | A | 176 | 133.855 | 23.229 | 16.112 | 1.00 | 29.43 | N | |
| ATOM | 1377 | CA | GLU | A | 176 | 133.716 | 23.179 | 17.550 | 1.00 | 29.98 | C | |
| ATOM | 1378 | C | GLU | A | 176 | 132.757 | 24.291 | 17.985 | 1.00 | 27.93 | C | |
| ATOM | 1379 | O | GLU | A | 176 | 131.832 | 24.054 | 18.803 | 1.00 | 31.72 | O | |
| ATOM | 1380 | CB | GLU | A | 176 | 135.097 | 23.395 | 18.176 | 1.00 | 32.55 | C | |
| ATOM | 1381 | CG | GLU | A | 176 | 135.181 | 23.165 | 19.642 | 1.00 | 39.70 | C | |
| ATOM | 1382 | CD | GLU | A | 176 | 136.526 | 23.643 | 20.181 | 1.00 | 46.95 | C | |
| ATOM | 1383 | OE1 | GLU | A | 176 | 137.587 | 23.313 | 19.550 | 1.00 | 46.01 | O | |
| ATOM | 1384 | OE2 | GLU | A | 176 | 136.509 | 24.357 | 21.228 | 1.00 | 51.19 | O | |
| ATOM | 1385 | N | LYS | A | 177 | 132.937 | 25.497 | 17.449 | 1.00 | 26.10 | N | |
| ATOM | 1386 | CA | LYS | A | 177 | 132.062 | 26.605 | 17.822 | 1.00 | 27.23 | C | |
| ATOM | 1387 | C | LYS | A | 177 | 130.621 | 26.326 | 17.342 | 1.00 | 28.94 | C | |
| ATOM | 1388 | O | LYS | A | 177 | 129.658 | 26.574 | 18.077 | 1.00 | 29.06 | O | |
| ATOM | 1389 | CB | LYS | A | 177 | 132.543 | 27.934 | 17.247 | 1.00 | 31.86 | C | |
| ATOM | 1390 | CG | LYS | A | 177 | 133.814 | 28.563 | 17.904 | 1.00 | 34.63 | C | |
| ATOM | 1391 | CD | LYS | A | 177 | 133.540 | 29.105 | 19.307 | 1.00 | 38.85 | C | |
| ATOM | 1392 | CE | LYS | A | 177 | 134.633 | 30.080 | 19.763 | 1.00 | 39.99 | C | |
| ATOM | 1393 | NZ | LYS | A | 177 | 134.620 | 30.318 | 21.227 | 1.00 | 43.91 | N | |
| ATOM | 1394 | N | VAL | A | 178 | 130.485 | 25.780 | 16.132 | 1.00 | 27.24 | N | |
| ATOM | 1395 | CA | VAL | A | 178 | 129.156 | 25.479 | 15.585 | 1.00 | 26.07 | C | |
| ATOM | 1396 | C | VAL | A | 178 | 128.437 | 24.410 | 16.426 | 1.00 | 24.57 | C | |
| ATOM | 1397 | O | VAL | A | 178 | 127.305 | 24.653 | 16.900 | 1.00 | 24.46 | O | |
| ATOM | 1398 | CB | VAL | A | 178 | 129.229 | 24.995 | 14.098 | 1.00 | 25.69 | C | |
| ATOM | 1399 | CG1 | VAL | A | 178 | 127.799 | 24.545 | 13.630 | 1.00 | 28.20 | C | |
| ATOM | 1400 | CG2 | VAL | A | 178 | 129.754 | 26.136 | 13.193 | 1.00 | 23.23 | C | |
| ATOM | 1401 | N | ATHR | A | 179 | 129.089 | 23.268 | 16.636 | 0.50 | 25.08 | N | |
| ATOM | 1402 | N | BTHR | A | 179 | 129.083 | 23.267 | 16.645 | 0.50 | 25.39 | N | |
| ATOM | 1403 | CA | ATHR | A | 179 | 128.477 | 22.189 | 17.413 | 0.50 | 27.71 | C | |
| ATOM | 1404 | CA | BTHR | A | 179 | 128.457 | 22.201 | 17.436 | 0.50 | 28.23 | C | |
| ATOM | 1405 | C | ATHR | A | 179 | 128.135 | 22.629 | 18.848 | 0.50 | 29.24 | C | |
| ATOM | 1406 | C | BTHR | A | 179 | 128.104 | 22.672 | 18.847 | 0.50 | 29.56 | C | |
| ATOM | 1407 | O | ATHR | A | 179 | 127.075 | 22.275 | 19.379 | 0.50 | 30.28 | O | |
| ATOM | 1408 | O | BTHR | A | 179 | 127.017 | 22.381 | 19.361 | 0.50 | 30.62 | O | |
| ATOM | 1409 | CB | ATHR | A | 179 | 129.385 | 20.919 | 17.461 | 0.50 | 26.44 | C | |
| ATOM | 1410 | CB | BTHR | A | 179 | 129.366 | 20.945 | 17.577 | 0.50 | 27.46 | C | |
| ATOM | 1411 | OG1 | ATHR | A | 179 | 129.763 | 20.527 | 16.135 | 0.50 | 27.60 | O | |
| ATOM | 1412 | OG1 | BTHR | A | 179 | 130.564 | 21.294 | 18.285 | 0.50 | 27.99 | O | |
| ATOM | 1413 | CG2 | ATHR | A | 179 | 128.635 | 19.770 | 18.065 | 0.50 | 24.77 | C | |
| ATOM | 1414 | CG2 | BTHR | A | 179 | 129.717 | 20.371 | 16.212 | 0.50 | 27.38 | C | |
| ATOM | 1415 | N | ASN | A | 180 | 129.021 | 23.395 | 19.478 | 1.00 | 31.21 | N | |
| ATOM | 1416 | CA | ASN | A | 180 | 128.776 | 23.889 | 20.844 | 1.00 | 33.89 | C | |
| ATOM | 1417 | C | ASN | A | 180 | 127.564 | 24.772 | 20.843 | 1.00 | 33.19 | C | |
| ATOM | 1418 | O | ASN | A | 180 | 126.754 | 24.716 | 21.764 | 1.00 | 37.55 | O | |
| ATOM | 1419 | CB | ASN | A | 180 | 129.940 | 24.749 | 21.374 | 1.00 | 34.53 | C | |
| ATOM | 1420 | CG | ASN | A | 180 | 129.690 | 25.268 | 22.810 | 1.00 | 40.24 | C | |
| ATOM | 1421 | OD1 | ASN | A | 180 | 129.641 | 24.483 | 23.771 | 1.00 | 45.30 | O | |
| ATOM | 1422 | ND2 | ASN | A | 180 | 129.526 | 26.586 | 22.953 | 1.00 | 40.51 | N | |
| ATOM | 1423 | N | SER | A | 181 | 127.448 | 25.621 | 19.824 | 1.00 | 30.38 | N | |
| ATOM | 1424 | CA | SER | A | 181 | 126.340 | 26.561 | 19.791 | 1.00 | 30.09 | C | |
| ATOM | 1425 | C | SER | A | 181 | 124.967 | 25.946 | 19.682 | 1.00 | 26.83 | C | |
| ATOM | 1426 | O | SER | A | 181 | 124.010 | 26.494 | 20.237 | 1.00 | 25.07 | O | |
| ATOM | 1427 | CB | SER | A | 181 | 126.520 | 27.565 | 18.669 | 1.00 | 32.68 | C | |
| ATOM | 1428 | OG | SER | A | 181 | 127.654 | 28.362 | 18.954 | 1.00 | 44.77 | O | |
| ATOM | 1429 | N | VAL | A | 182 | 124.874 | 24.829 | 18.970 | 1.00 | 24.75 | N | |
| ATOM | 1430 | CA | VAL | A | 182 | 123.593 | 24.142 | 18.830 | 1.00 | 27.22 | C | |
| ATOM | 1431 | C | VAL | A | 182 | 123.095 | 23.607 | 20.198 | 1.00 | 26.25 | C | |
| ATOM | 1432 | O | VAL | A | 182 | 121.943 | 23.821 | 20.588 | 1.00 | 23.16 | O | |
| ATOM | 1433 | CB | VAL | A | 182 | 123.716 | 22.981 | 17.781 | 1.00 | 27.55 | C | |
| ATOM | 1434 | CG1 | VAL | A | 182 | 122.428 | 22.142 | 17.724 | 1.00 | 26.04 | C | |
| ATOM | 1435 | CG2 | VAL | A | 182 | 124.009 | 23.584 | 16.385 | 1.00 | 26.42 | C | |
| ATOM | 1436 | N | ASER | A | 183 | 123.983 | 22.936 | 20.925 | 0.50 | 26.36 | N | |
| ATOM | 1437 | N | BSER | A | 183 | 123.959 | 22.927 | 20.940 | 0.50 | 26.00 | N | |
| ATOM | 1438 | CA | ASER | A | 183 | 123.641 | 22.358 | 22.222 | 0.50 | 28.23 | C | |
| ATOM | 1439 | CA | BSER | A | 183 | 123.526 | 22.388 | 22.224 | 0.50 | 27.45 | C | |
| ATOM | 1440 | C | ASER | A | 183 | 123.429 | 23.413 | 23.310 | 0.50 | 28.30 | C | |
| ATOM | 1441 | C | BSER | A | 183 | 123.372 | 23.465 | 23.300 | 0.50 | 27.70 | C | |
| ATOM | 1442 | O | ASER | A | 183 | 122.467 | 23.351 | 24.090 | 0.50 | 26.71 | O | |
| ATOM | 1443 | O | BSER | A | 183 | 122.394 | 23.471 | 24.064 | 0.50 | 24.65 | O | |
| ATOM | 1444 | CB | ASER | A | 183 | 124.746 | 21.378 | 22.649 | 0.50 | 29.05 | C | |
| ATOM | 1445 | CB | BSER | A | 183 | 124.498 | 21.302 | 22.689 | 0.50 | 28.26 | C | |
| ATOM | 1446 | OG | ASER | A | 183 | 124.402 | 20.669 | 23.833 | 0.50 | 31.43 | O | |
| ATOM | 1447 | OG | BSER | A | 183 | 125.796 | 21.825 | 22.866 | 0.50 | 27.29 | O | |
| ATOM | 1448 | N | SER | A | 184 | 124.303 | 24.411 | 23.326 | 1.00 | 27.41 | N | |
| ATOM | 1449 | CA | SER | A | 184 | 124.258 | 25.444 | 24.346 | 1.00 | 28.94 | C | |
| ATOM | 1450 | C | SER | A | 184 | 123.401 | 26.686 | 24.122 | 1.00 | 27.27 | C | |
| ATOM | 1451 | O | SER | A | 184 | 123.108 | 27.423 | 25.072 | 1.00 | 28.49 | O | |
| ATOM | 1452 | CB | SER | A | 184 | 125.687 | 25.894 | 24.650 | 1.00 | 32.28 | C | |
| ATOM | 1453 | OG | SER | A | 184 | 126.061 | 26.954 | 23.799 | 1.00 | 31.90 | O | |
| ATOM | 1454 | N | GLU | A | 185 | 122.974 | 26.934 | 22.900 | 1.00 | 24.34 | N | |
| ATOM | 1455 | CA | GLU | A | 185 | 122.196 | 28.140 | 22.669 | 1.00 | 21.04 | C | |
| ATOM | 1456 | C | GLU | A | 185 | 120.962 | 27.860 | 21.800 | 1.00 | 22.31 | C | |
| ATOM | 1457 | O | GLU | A | 185 | 119.865 | 28.289 | 22.133 | 1.00 | 22.95 | O | |
| ATOM | 1458 | CB | GLU | A | 185 | 123.098 | 29.178 | 21.985 | 1.00 | 23.67 | C | |
| ATOM | 1459 | CG | GLU | A | 185 | 124.395 | 29.349 | 22.763 | 1.00 | 31.02 | C | |
| ATOM | 1460 | CD | GLU | A | 185 | 125.321 | 30.431 | 22.213 | 1.00 | 35.91 | C | |
| ATOM | 1461 | OE1 | GLU | A | 185 | 125.152 | 30.874 | 21.046 | 1.00 | 38.95 | O | |
| ATOM | 1462 | OE2 | GLU | A | 185 | 126.237 | 30.833 | 22.970 | 1.00 | 41.06 | O | |
| ATOM | 1463 | N | LEU | A | 186 | 121.146 | 27.101 | 20.732 | 1.00 | 23.00 | N | |
| ATOM | 1464 | CA | LEU | A | 186 | 120.033 | 26.809 | 19.810 | 1.00 | 23.22 | C | |
| ATOM | 1465 | C | LEU | A | 186 | 118.932 | 25.955 | 20.421 | 1.00 | 20.40 | C | |
| ATOM | 1466 | O | LEU | A | 186 | 117.769 | 26.330 | 20.342 | 1.00 | 22.38 | O | |
| ATOM | 1467 | CB | LEU | A | 186 | 120.537 | 26.138 | 18.530 | 1.00 | 22.39 | C | |
| ATOM | 1468 | CG | LEU | A | 186 | 120.052 | 26.544 | 17.116 | 1.00 | 32.01 | C | |
| ATOM | 1469 | CD1 | LEU | A | 186 | 119.963 | 25.343 | 16.240 | 1.00 | 25.73 | C | |
| ATOM | 1470 | CD2 | LEU | A | 186 | 118.754 | 27.262 | 17.135 | 1.00 | 26.16 | C | |
| ATOM | 1471 | N | GLN | A | 187 | 119.263 | 24.810 | 21.009 | 1.00 | 23.18 | N | |
| ATOM | 1472 | CA | GLN | A | 187 | 118.212 | 23.986 | 21.597 | 1.00 | 24.27 | C | |
| ATOM | 1473 | C | GLN | A | 187 | 117.475 | 24.717 | 22.737 | 1.00 | 25.94 | C | |
| ATOM | 1474 | O | GLN | A | 187 | 116.237 | 24.698 | 22.818 | 1.00 | 23.84 | O | |
| ATOM | 1475 | CB | GLN | A | 187 | 118.761 | 22.645 | 22.103 | 1.00 | 27.40 | C | |
| ATOM | 1476 | CG | GLN | A | 187 | 117.744 | 21.841 | 22.874 | 1.00 | 26.56 | C | |
| ATOM | 1477 | CD | GLN | A | 187 | 118.127 | 20.364 | 23.008 | 1.00 | 31.86 | C | |
| ATOM | 1478 | OE1 | GLN | A | 187 | 117.914 | 19.572 | 22.114 | 1.00 | 29.13 | O | |
| ATOM | 1479 | NE2 | GLN | A | 187 | 118.700 | 19.997 | 24.169 | 1.00 | 33.25 | N | |
| ATOM | 1480 | N | PRO | A | 188 | 118.222 | 25.345 | 23.646 | 1.00 | 26.18 | N | |
| ATOM | 1481 | CA | PRO | A | 188 | 117.562 | 26.069 | 24.741 | 1.00 | 27.85 | C | |
| ATOM | 1482 | C | PRO | A | 188 | 116.657 | 27.168 | 24.198 | 1.00 | 24.86 | C | |
| ATOM | 1483 | O | PRO | A | 188 | 115.612 | 27.430 | 24.768 | 1.00 | 25.50 | O | |
| ATOM | 1484 | CB | PRO | A | 188 | 118.737 | 26.655 | 25.520 | 1.00 | 27.16 | C | |
| ATOM | 1485 | CG | PRO | A | 188 | 119.797 | 25.592 | 25.355 | 1.00 | 27.45 | C | |
| ATOM | 1486 | CD | PRO | A | 188 | 119.673 | 25.236 | 23.885 | 1.00 | 30.03 | C | |
| ATOM | 1487 | N | TYR | A | 189 | 117.074 | 27.815 | 23.102 | 1.00 | 24.19 | N | |
| ATOM | 1488 | CA | TYR | A | 189 | 116.256 | 28.876 | 22.502 | 1.00 | 24.40 | C | |
| ATOM | 1489 | C | TYR | A | 189 | 114.927 | 28.296 | 22.034 | 1.00 | 25.09 | C | |
| ATOM | 1490 | O | TYR | A | 189 | 113.852 | 28.780 | 22.414 | 1.00 | 23.23 | O | |
| ATOM | 1491 | CB | TYR | A | 189 | 116.914 | 29.550 | 21.275 | 1.00 | 21.89 | C | |
| ATOM | 1492 | CG | TYR | A | 189 | 115.983 | 30.591 | 20.620 | 1.00 | 22.82 | C | |
| ATOM | 1493 | CD1 | TYR | A | 189 | 115.647 | 31.783 | 21.280 | 1.00 | 24.28 | C | |
| ATOM | 1494 | CD2 | TYR | A | 189 | 115.311 | 30.291 | 19.444 | 1.00 | 21.73 | C | |
| ATOM | 1495 | CE1 | TYR | A | 189 | 114.637 | 32.647 | 20.784 | 1.00 | 23.47 | C | |
| ATOM | 1496 | CE2 | TYR | A | 189 | 114.315 | 31.122 | 18.948 | 1.00 | 23.03 | C | |
| ATOM | 1497 | CZ | TYR | A | 189 | 113.976 | 32.311 | 19.632 | 1.00 | 25.17 | C | |
| ATOM | 1498 | OH | TYR | A | 189 | 112.971 | 33.100 | 19.123 | 1.00 | 25.01 | O | |
| ATOM | 1499 | N | PHE | A | 190 | 114.967 | 27.240 | 21.236 | 1.00 | 22.83 | N | |
| ATOM | 1500 | CA | PHE | A | 190 | 113.680 | 26.751 | 20.775 | 1.00 | 25.66 | C | |
| ATOM | 1501 | C | PHE | A | 190 | 112.844 | 26.143 | 21.929 | 1.00 | 23.67 | C | |
| ATOM | 1502 | O | PHE | A | 190 | 111.605 | 26.099 | 21.827 | 1.00 | 22.61 | O | |
| ATOM | 1503 | CB | PHE | A | 190 | 113.857 | 25.855 | 19.525 | 1.00 | 26.98 | C | |
| ATOM | 1504 | CG | PHE | A | 190 | 113.938 | 26.667 | 18.212 | 1.00 | 27.82 | C | |
| ATOM | 1505 | CD1 | PHE | A | 190 | 112.795 | 27.252 | 17.659 | 1.00 | 27.22 | C | |
| ATOM | 1506 | CD2 | PHE | A | 190 | 115.171 | 26.932 | 17.604 | 1.00 | 28.89 | C | |
| ATOM | 1507 | CE1 | PHE | A | 190 | 112.857 | 28.104 | 16.512 | 1.00 | 26.14 | C | |
| ATOM | 1508 | CE2 | PHE | A | 190 | 115.266 | 27.788 | 16.452 | 1.00 | 26.83 | C | |
| ATOM | 1509 | CZ | PHE | A | 190 | 114.090 | 28.377 | 15.905 | 1.00 | 23.06 | C | |
| ATOM | 1510 | N | GLN | A | 191 | 113.503 | 25.740 | 23.020 | 1.00 | 23.88 | N | |
| ATOM | 1511 | CA | GLN | A | 191 | 112.805 | 25.253 | 24.197 | 1.00 | 23.48 | C | |
| ATOM | 1512 | C | GLN | A | 191 | 112.121 | 26.414 | 24.966 | 1.00 | 22.32 | C | |
| ATOM | 1513 | O | GLN | A | 191 | 111.507 | 26.182 | 26.001 | 1.00 | 20.88 | O | |
| ATOM | 1514 | CB | GLN | A | 191 | 113.729 | 24.432 | 25.109 | 1.00 | 24.84 | C | |
| ATOM | 1515 | CG | GLN | A | 191 | 114.021 | 23.078 | 24.461 | 1.00 | 24.73 | C | |
| ATOM | 1516 | CD | GLN | A | 191 | 114.605 | 22.030 | 25.396 | 1.00 | 29.87 | C | |
| ATOM | 1517 | OE1 | GLN | A | 191 | 115.072 | 22.341 | 26.485 | 1.00 | 30.16 | O | |
| ATOM | 1518 | NE2 | GLN | A | 191 | 114.578 | 20.761 | 24.958 | 1.00 | 25.66 | N | |
| ATOM | 1519 | N | THR | A | 192 | 112.203 | 27.635 | 24.436 | 1.00 | 21.04 | N | |
| ATOM | 1520 | CA | THR | A | 192 | 111.444 | 28.746 | 25.033 | 1.00 | 20.04 | C | |
| ATOM | 1521 | C | THR | A | 192 | 110.022 | 28.750 | 24.488 | 1.00 | 20.79 | C | |
| ATOM | 1522 | O | THR | A | 192 | 109.168 | 29.552 | 24.890 | 1.00 | 19.72 | O | |
| ATOM | 1523 | CB | THR | A | 192 | 112.069 | 30.153 | 24.807 | 1.00 | 19.73 | C | |
| ATOM | 1524 | OG1 | THR | A | 192 | 112.078 | 30.526 | 23.414 | 1.00 | 20.39 | O | |
| ATOM | 1525 | CG2 | THR | A | 192 | 113.474 | 30.168 | 25.437 | 1.00 | 20.70 | C | |
| ATOM | 1526 | N | LEU | A | 193 | 109.749 | 27.849 | 23.559 | 1.00 | 21.88 | N | |
| ATOM | 1527 | CA | LEU | A | 193 | 108.363 | 27.717 | 23.074 | 1.00 | 22.29 | C | |
| ATOM | 1528 | C | LEU | A | 193 | 107.538 | 27.373 | 24.340 | 1.00 | 20.07 | C | |
| ATOM | 1529 | O | LEU | A | 193 | 107.958 | 26.567 | 25.179 | 1.00 | 21.77 | O | |
| ATOM | 1530 | CB | LEU | A | 193 | 108.220 | 26.547 | 22.066 | 1.00 | 18.23 | C | |
| ATOM | 1531 | CG | LEU | A | 193 | 108.598 | 26.796 | 20.603 | 1.00 | 24.99 | C | |
| ATOM | 1532 | CD1 | LEU | A | 193 | 108.401 | 25.540 | 19.781 | 1.00 | 20.37 | C | |
| ATOM | 1533 | CD2 | LEU | A | 193 | 107.721 | 27.881 | 20.053 | 1.00 | 19.89 | C | |
| ATOM | 1534 | N | PRO | A | 194 | 106.349 | 27.986 | 24.506 | 1.00 | 20.29 | N | |
| ATOM | 1535 | CA | PRO | A | 194 | 105.579 | 27.639 | 25.714 | 1.00 | 19.31 | C | |
| ATOM | 1536 | C | PRO | A | 194 | 104.887 | 26.272 | 25.654 | 1.00 | 18.13 | C | |
| ATOM | 1537 | O | PRO | A | 194 | 104.413 | 25.859 | 24.597 | 1.00 | 21.37 | O | |
| ATOM | 1538 | CB | PRO | A | 194 | 104.549 | 28.786 | 25.835 | 1.00 | 21.54 | C | |
| ATOM | 1539 | CG | PRO | A | 194 | 104.305 | 29.156 | 24.353 | 1.00 | 22.99 | C | |
| ATOM | 1540 | CD | PRO | A | 194 | 105.737 | 29.108 | 23.750 | 1.00 | 20.06 | C | |
| ATOM | 1541 | N | VAL | A | 195 | 104.866 | 25.560 | 26.778 | 1.00 | 19.82 | N | |
| ATOM | 1542 | CA | VAL | A | 195 | 104.107 | 24.284 | 26.788 | 1.00 | 21.94 | C | |
| ATOM | 1543 | C | VAL | A | 195 | 102.730 | 24.455 | 27.446 | 1.00 | 23.92 | C | |
| ATOM | 1544 | O | VAL | A | 195 | 101.717 | 24.441 | 26.732 | 1.00 | 25.89 | O | |
| ATOM | 1545 | CB | VAL | A | 195 | 104.850 | 23.161 | 27.490 | 1.00 | 19.83 | C | |
| ATOM | 1546 | CG1 | VAL | A | 195 | 103.989 | 21.906 | 27.454 | 1.00 | 22.29 | C | |
| ATOM | 1547 | CG2 | VAL | A | 195 | 106.200 | 22.924 | 26.772 | 1.00 | 23.48 | C | |
| ATOM | 1548 | N | MET | A | 196 | 102.693 | 24.559 | 28.785 | 1.00 | 25.99 | N | |
| ATOM | 1549 | CA | MET | A | 196 | 101.432 | 24.788 | 29.561 | 1.00 | 25.86 | C | |
| ATOM | 1550 | C | MET | A | 196 | 101.415 | 26.322 | 29.766 | 1.00 | 27.21 | C | |
| ATOM | 1551 | O | MET | A | 196 | 102.098 | 26.848 | 30.662 | 1.00 | 28.87 | O | |
| ATOM | 1552 | CB | MET | A | 196 | 101.478 | 24.077 | 30.925 | 1.00 | 33.30 | C | |
| ATOM | 1553 | CG | MET | A | 196 | 101.628 | 22.541 | 30.859 | 1.00 | 30.15 | C | |
| ATOM | 1554 | SD | MET | A | 196 | 100.318 | 21.663 | 29.891 | 1.00 | 37.31 | S | |
| ATOM | 1555 | CE | MET | A | 196 | 99.171 | 21.210 | 31.186 | 1.00 | 36.65 | C | |
| ATOM | 1556 | N | THR | A | 197 | 100.673 | 27.043 | 28.922 | 1.00 | 26.18 | N | |
| ATOM | 1557 | CA | THR | A | 197 | 100.637 | 28.521 | 28.955 | 1.00 | 23.64 | C | |
| ATOM | 1558 | C | THR | A | 197 | 99.353 | 29.083 | 29.621 | 1.00 | 22.70 | C | |
| ATOM | 1559 | O | THR | A | 197 | 98.235 | 29.054 | 29.081 | 1.00 | 17.43 | O | |
| ATOM | 1560 | CB | THR | A | 197 | 101.011 | 29.033 | 27.507 | 1.00 | 32.57 | C | |
| ATOM | 1561 | OG1 | THR | A | 197 | 100.038 | 29.924 | 26.955 | 1.00 | 32.53 | O | |
| ATOM | 1562 | CG2 | THR | A | 197 | 101.261 | 27.799 | 26.606 | 1.00 | 21.87 | C | |
| ATOM | 1563 | N | LYS | A | 198 | 99.538 | 29.560 | 30.855 | 1.00 | 17.69 | N | |
| ATOM | 1564 | CA | LYS | A | 198 | 98.411 | 30.034 | 31.693 | 1.00 | 16.55 | C | |
| ATOM | 1565 | C | LYS | A | 198 | 97.832 | 31.370 | 31.225 | 1.00 | 17.07 | C | |
| ATOM | 1566 | O | LYS | A | 198 | 98.548 | 32.344 | 30.963 | 1.00 | 20.17 | O | |
| ATOM | 1567 | CB | LYS | A | 198 | 98.872 | 30.109 | 33.183 | 1.00 | 15.78 | C | |
| ATOM | 1568 | CG | LYS | A | 198 | 97.725 | 30.365 | 34.171 | 1.00 | 17.55 | C | |
| ATOM | 1569 | CD | LYS | A | 198 | 98.246 | 30.535 | 35.602 | 1.00 | 19.54 | C | |
| ATOM | 1570 | CE | LYS | A | 198 | 97.130 | 30.887 | 36.569 | 1.00 | 22.02 | C | |
| ATOM | 1571 | NZ | LYS | A | 198 | 96.236 | 29.695 | 36.849 | 1.00 | 26.99 | N | |
| ATOM | 1572 | N | ILE | A | 199 | 96.517 | 31.421 | 31.110 | 1.00 | 17.09 | N | |
| ATOM | 1573 | CA | ILE | A | 199 | 95.817 | 32.605 | 30.650 | 1.00 | 18.15 | C | |
| ATOM | 1574 | C | ILE | A | 199 | 95.323 | 33.481 | 31.801 | 1.00 | 19.51 | C | |
| ATOM | 1575 | O | ILE | A | 199 | 95.517 | 34.713 | 31.831 | 1.00 | 21.21 | O | |
| ATOM | 1576 | CB | ILE | A | 199 | 94.517 | 32.199 | 29.819 | 1.00 | 18.17 | C | |
| ATOM | 1577 | CG1 | ILE | A | 199 | 94.871 | 31.287 | 28.645 | 1.00 | 23.48 | C | |
| ATOM | 1578 | CG2 | ILE | A | 199 | 93.760 | 33.441 | 29.384 | 1.00 | 17.24 | C | |
| ATOM | 1579 | CD1 | ILE | A | 199 | 93.546 | 30.605 | 28.074 | 1.00 | 17.53 | C | |
| ATOM | 1580 | N | ASP | A | 200 | 94.653 | 32.837 | 32.741 | 1.00 | 14.93 | N | |
| ATOM | 1581 | CA | ASP | A | 200 | 94.013 | 33.508 | 33.864 | 1.00 | 17.10 | C | |
| ATOM | 1582 | C | ASP | A | 200 | 93.783 | 32.502 | 34.976 | 1.00 | 19.63 | C | |
| ATOM | 1583 | O | ASP | A | 200 | 94.344 | 31.399 | 34.924 | 1.00 | 19.11 | O | |
| ATOM | 1584 | CB | ASP | A | 200 | 92.668 | 34.097 | 33.412 | 1.00 | 17.71 | C | |
| ATOM | 1585 | CG | ASP | A | 200 | 91.800 | 33.089 | 32.704 | 1.00 | 20.60 | C | |
| ATOM | 1586 | OD1 | ASP | A | 200 | 91.787 | 31.918 | 33.118 | 1.00 | 18.71 | O | |
| ATOM | 1587 | OD2 | ASP | A | 200 | 91.115 | 33.470 | 31.740 | 1.00 | 19.20 | O | |
| ATOM | 1588 | N | SER | A | 201 | 92.944 | 32.854 | 35.951 | 1.00 | 19.02 | N | |
| ATOM | 1589 | CA | SER | A | 201 | 92.747 | 31.932 | 37.093 | 1.00 | 20.50 | C | |
| ATOM | 1590 | C | SER | A | 201 | 91.902 | 30.738 | 36.817 | 1.00 | 19.77 | C | |
| ATOM | 1591 | O | SER | A | 201 | 91.754 | 29.864 | 37.688 | 1.00 | 23.22 | O | |
| ATOM | 1592 | CB | SER | A | 201 | 92.176 | 32.660 | 38.315 | 1.00 | 21.90 | C | |
| ATOM | 1593 | OG | SER | A | 201 | 90.866 | 33.134 | 38.076 | 1.00 | 19.95 | O | |
| ATOM | 1594 | N | VAL | A | 202 | 91.378 | 30.660 | 35.595 | 1.00 | 18.29 | N | |
| ATOM | 1595 | CA | VAL | A | 202 | 90.518 | 29.553 | 35.208 | 1.00 | 16.71 | C | |
| ATOM | 1596 | C | VAL | A | 202 | 91.098 | 28.589 | 34.166 | 1.00 | 17.73 | C | |
| ATOM | 1597 | O | VAL | A | 202 | 90.909 | 27.367 | 34.300 | 1.00 | 17.56 | O | |
| ATOM | 1598 | CB | VAL | A | 202 | 89.213 | 30.101 | 34.663 | 1.00 | 19.74 | C | |
| ATOM | 1599 | CG1 | VAL | A | 202 | 88.388 | 29.021 | 34.002 | 1.00 | 23.46 | C | |
| ATOM | 1600 | CG2 | VAL | A | 202 | 88.416 | 30.683 | 35.864 | 1.00 | 23.83 | C | |
| ATOM | 1601 | N | ALA | A | 203 | 91.799 | 29.129 | 33.168 | 1.00 | 16.82 | N | |
| ATOM | 1602 | CA | ALA | A | 203 | 92.320 | 28.284 | 32.087 | 1.00 | 15.01 | C | |
| ATOM | 1603 | C | ALA | A | 203 | 93.738 | 28.505 | 31.640 | 1.00 | 17.70 | C | |
| ATOM | 1604 | O | ALA | A | 203 | 94.352 | 29.550 | 31.914 | 1.00 | 15.33 | O | |
| ATOM | 1605 | CB | ALA | A | 203 | 91.431 | 28.452 | 30.892 | 1.00 | 18.30 | C | |
| ATOM | 1606 | N | GLY | A | 204 | 94.223 | 27.513 | 30.913 | 1.00 | 15.41 | N | |
| ATOM | 1607 | CA | GLY | A | 204 | 95.546 | 27.565 | 30.281 | 1.00 | 17.66 | C | |
| ATOM | 1608 | C | GLY | A | 204 | 95.391 | 26.908 | 28.887 | 1.00 | 16.76 | C | |
| ATOM | 1609 | O | GLY | A | 204 | 94.319 | 26.382 | 28.567 | 1.00 | 14.75 | O | |
| ATOM | 1610 | N | ILE | A | 205 | 96.421 | 26.992 | 28.065 | 1.00 | 14.46 | N | |
| ATOM | 1611 | CA | ILE | A | 205 | 96.427 | 26.341 | 26.766 | 1.00 | 15.49 | C | |
| ATOM | 1612 | C | ILE | A | 205 | 97.642 | 25.420 | 26.698 | 1.00 | 16.26 | C | |
| ATOM | 1613 | O | ILE | A | 205 | 98.746 | 25.843 | 27.028 | 1.00 | 16.31 | O | |
| ATOM | 1614 | CB | ILE | A | 205 | 96.581 | 27.341 | 25.614 | 1.00 | 15.07 | C | |
| ATOM | 1615 | CG1 | ILE | A | 205 | 95.351 | 28.267 | 25.553 | 1.00 | 15.49 | C | |
| ATOM | 1616 | CG2 | ILE | A | 205 | 96.678 | 26.603 | 24.249 | 1.00 | 17.51 | C | |
| ATOM | 1617 | CD1 | ILE | A | 205 | 95.616 | 29.536 | 24.641 | 1.00 | 14.72 | C | |
| ATOM | 1618 | N | ASN | A | 206 | 97.426 | 24.158 | 26.303 | 1.00 | 15.74 | N | |
| ATOM | 1619 | CA | ASN | A | 206 | 98.512 | 23.200 | 26.170 | 1.00 | 19.28 | C | |
| ATOM | 1620 | C | ASN | A | 206 | 99.020 | 23.242 | 24.704 | 1.00 | 17.73 | C | |
| ATOM | 1621 | O | ASN | A | 206 | 98.299 | 22.844 | 23.764 | 1.00 | 15.25 | O | |
| ATOM | 1622 | CB | ASN | A | 206 | 97.994 | 21.796 | 26.527 | 1.00 | 17.99 | C | |
| ATOM | 1623 | CG | ASN | A | 206 | 99.107 | 20.712 | 26.511 | 1.00 | 21.23 | C | |
| ATOM | 1624 | OD1 | ASN | A | 206 | 100.193 | 20.913 | 25.998 | 1.00 | 20.88 | O | |
| ATOM | 1625 | ND2 | ASN | A | 206 | 98.775 | 19.537 | 27.105 | 1.00 | 24.75 | N | |
| ATOM | 1626 | N | TYR | A | 207 | 100.220 | 23.775 | 24.523 | 1.00 | 14.46 | N | |
| ATOM | 1627 | CA | TYR | A | 207 | 100.857 | 23.902 | 23.221 | 1.00 | 14.21 | C | |
| ATOM | 1628 | C | TYR | A | 207 | 102.011 | 22.894 | 23.081 | 1.00 | 15.08 | C | |
| ATOM | 1629 | O | TYR | A | 207 | 102.853 | 23.025 | 22.195 | 1.00 | 15.16 | O | |
| ATOM | 1630 | CB | TYR | A | 207 | 101.414 | 25.349 | 23.000 | 1.00 | 16.89 | C | |
| ATOM | 1631 | CG | TYR | A | 207 | 100.363 | 26.418 | 22.673 | 1.00 | 15.13 | C | |
| ATOM | 1632 | CD1 | TYR | A | 207 | 99.542 | 26.325 | 21.538 | 1.00 | 17.14 | C | |
| ATOM | 1633 | CD2 | TYR | A | 207 | 100.269 | 27.586 | 23.447 | 1.00 | 18.54 | C | |
| ATOM | 1634 | CE1 | TYR | A | 207 | 98.663 | 27.379 | 21.163 | 1.00 | 15.11 | C | |
| ATOM | 1635 | CE2 | TYR | A | 207 | 99.386 | 28.620 | 23.085 | 1.00 | 16.50 | C | |
| ATOM | 1636 | CZ | TYR | A | 207 | 98.597 | 28.516 | 21.946 | 1.00 | 17.52 | C | |
| ATOM | 1637 | OH | TYR | A | 207 | 97.757 | 29.548 | 21.585 | 1.00 | 17.06 | O | |
| ATOM | 1638 | N | GLY | A | 208 | 102.046 | 21.903 | 23.964 | 1.00 | 17.19 | N | |
| ATOM | 1639 | CA | GLY | A | 208 | 103.128 | 20.895 | 23.876 | 1.00 | 18.13 | C | |
| ATOM | 1640 | C | GLY | A | 208 | 103.155 | 20.147 | 22.569 | 1.00 | 17.53 | C | |
| ATOM | 1641 | O | GLY | A | 208 | 102.126 | 19.901 | 21.998 | 1.00 | 17.81 | O | |
| ATOM | 1642 | N | LEU | A | 209 | 104.350 | 19.778 | 22.078 | 1.00 | 16.77 | N | |
| ATOM | 1643 | CA | LEU | A | 209 | 104.405 | 19.066 | 20.785 | 1.00 | 13.76 | C | |
| ATOM | 1644 | C | LEU | A | 209 | 103.855 | 17.658 | 20.979 | 1.00 | 17.46 | C | |
| ATOM | 1645 | O | LEU | A | 209 | 104.145 | 17.050 | 22.020 | 1.00 | 19.02 | O | |
| ATOM | 1646 | CB | LEU | A | 209 | 105.858 | 18.984 | 20.316 | 1.00 | 16.90 | C | |
| ATOM | 1647 | CG | LEU | A | 209 | 106.408 | 20.113 | 19.475 | 1.00 | 18.69 | C | |
| ATOM | 1648 | CD1 | LEU | A | 209 | 106.339 | 21.435 | 20.246 | 1.00 | 20.90 | C | |
| ATOM | 1649 | CD2 | LEU | A | 209 | 107.906 | 19.758 | 19.106 | 1.00 | 19.97 | C | |
| ATOM | 1650 | N | VAL | A | 210 | 103.057 | 17.195 | 20.011 | 1.00 | 17.56 | N | |
| ATOM | 1651 | CA | VAL | A | 210 | 102.472 | 15.830 | 20.076 | 1.00 | 16.69 | C | |
| ATOM | 1652 | C | VAL | A | 210 | 103.223 | 14.906 | 19.091 | 1.00 | 21.90 | C | |
| ATOM | 1653 | O | VAL | A | 210 | 102.866 | 13.737 | 18.938 | 1.00 | 18.52 | O | |
| ATOM | 1654 | CB | VAL | A | 210 | 100.921 | 15.844 | 19.764 | 1.00 | 20.62 | C | |
| ATOM | 1655 | CG1 | VAL | A | 210 | 100.185 | 16.492 | 20.932 | 1.00 | 19.52 | C | |
| ATOM | 1656 | CG2 | VAL | A | 210 | 100.610 | 16.606 | 18.449 | 1.00 | 20.27 | C | |
| ATOM | 1657 | N | ALA | A | 211 | 104.240 | 15.448 | 18.414 | 1.00 | 19.94 | N | |
| ATOM | 1658 | CA | ALA | A | 211 | 105.082 | 14.678 | 17.465 | 1.00 | 19.38 | C | |
| ATOM | 1659 | C | ALA | A | 211 | 106.375 | 15.411 | 17.220 | 1.00 | 21.07 | C | |
| ATOM | 1660 | O | ALA | A | 211 | 106.471 | 16.601 | 17.467 | 1.00 | 19.58 | O | |
| ATOM | 1661 | CB | ALA | A | 211 | 104.349 | 14.514 | 16.125 | 1.00 | 21.45 | C | |
| ATOM | 1662 | N | PRO | A | 212 | 107.419 | 14.715 | 16.741 | 1.00 | 20.58 | N | |
| ATOM | 1663 | CA | PRO | A | 212 | 108.653 | 15.470 | 16.487 | 1.00 | 19.71 | C | |
| ATOM | 1664 | C | PRO | A | 212 | 108.469 | 16.327 | 15.214 | 1.00 | 20.34 | C | |
| ATOM | 1665 | O | PRO | A | 212 | 107.695 | 15.978 | 14.353 | 1.00 | 18.95 | O | |
| ATOM | 1666 | CB | PRO | A | 212 | 109.718 | 14.359 | 16.367 | 1.00 | 26.23 | C | |
| ATOM | 1667 | CG | PRO | A | 212 | 108.931 | 13.159 | 15.899 | 1.00 | 22.32 | C | |
| ATOM | 1668 | CD | PRO | A | 212 | 107.562 | 13.262 | 16.492 | 1.00 | 21.94 | C | |
| ATOM | 1669 | N | PRO | A | 213 | 109.139 | 17.489 | 15.113 | 1.00 | 19.28 | N | |
| ATOM | 1670 | CA | PRO | A | 213 | 109.004 | 18.345 | 13.925 | 1.00 | 19.81 | C | |
| ATOM | 1671 | C | PRO | A | 213 | 109.322 | 17.540 | 12.660 | 1.00 | 20.67 | C | |
| ATOM | 1672 | O | PRO | A | 213 | 110.345 | 16.856 | 12.620 | 1.00 | 22.30 | O | |
| ATOM | 1673 | CB | PRO | A | 213 | 110.045 | 19.451 | 14.144 | 1.00 | 21.27 | C | |
| ATOM | 1674 | CG | PRO | A | 213 | 110.148 | 19.542 | 15.652 | 1.00 | 21.58 | C | |
| ATOM | 1675 | CD | PRO | A | 213 | 110.076 | 18.058 | 16.093 | 1.00 | 19.24 | C | |
| ATOM | 1676 | N | ALA | A | 214 | 108.465 | 17.631 | 11.650 | 1.00 | 20.43 | N | |
| ATOM | 1677 | CA | ALA | A | 214 | 108.639 | 16.855 | 10.428 | 1.00 | 21.64 | C | |
| ATOM | 1678 | C | ALA | A | 214 | 109.039 | 17.757 | 9.248 | 1.00 | 24.37 | C | |
| ATOM | 1679 | O | ALA | A | 214 | 108.352 | 18.730 | 8.963 | 1.00 | 22.63 | O | |
| ATOM | 1680 | CB | ALA | A | 214 | 107.343 | 16.110 | 10.096 | 1.00 | 26.85 | C | |
| ATOM | 1681 | N | THR | A | 215 | 110.115 | 17.382 | 8.551 | 1.00 | 20.66 | N | |
| ATOM | 1682 | CA | THR | A | 215 | 110.647 | 18.158 | 7.432 | 1.00 | 22.28 | C | |
| ATOM | 1683 | C | THR | A | 215 | 110.241 | 17.548 | 6.096 | 1.00 | 21.26 | C | |
| ATOM | 1684 | O | THR | A | 215 | 110.400 | 16.324 | 5.909 | 1.00 | 20.02 | O | |
| ATOM | 1685 | CB | THR | A | 215 | 112.178 | 18.180 | 7.515 | 1.00 | 21.14 | C | |
| ATOM | 1686 | OG1 | THR | A | 215 | 112.576 | 18.742 | 8.773 | 1.00 | 22.37 | O | |
| ATOM | 1687 | CG2 | THR | A | 215 | 112.792 | 19.012 | 6.385 | 1.00 | 24.68 | C | |
| ATOM | 1688 | N | THR | A | 216 | 109.652 | 18.374 | 5.225 | 1.00 | 21.25 | N | |
| ATOM | 1689 | CA | THR | A | 216 | 109.290 | 17.968 | 3.872 | 1.00 | 21.70 | C | |
| ATOM | 1690 | C | THR | A | 216 | 110.068 | 18.865 | 2.909 | 1.00 | 19.85 | C | |
| ATOM | 1691 | O | THR | A | 216 | 110.931 | 19.624 | 3.366 | 1.00 | 21.33 | O | |
| ATOM | 1692 | CB | THR | A | 216 | 107.786 | 18.073 | 3.614 | 1.00 | 23.72 | C | |
| ATOM | 1693 | OG1 | THR | A | 216 | 107.414 | 19.455 | 3.579 | 1.00 | 24.65 | O | |
| ATOM | 1694 | CG2 | THR | A | 216 | 106.999 | 17.317 | 4.720 | 1.00 | 23.72 | C | |
| ATOM | 1695 | N | ALA | A | 217 | 109.808 | 18.778 | 1.593 | 1.00 | 19.35 | N | |
| ATOM | 1696 | CA | ALA | A | 217 | 110.580 | 19.572 | 0.635 | 1.00 | 21.35 | C | |
| ATOM | 1697 | C | ALA | A | 217 | 110.355 | 21.053 | 0.801 | 1.00 | 22.61 | C | |
| ATOM | 1698 | O | ALA | A | 217 | 111.255 | 21.874 | 0.571 | 1.00 | 21.02 | O | |
| ATOM | 1699 | CB | ALA | A | 217 | 110.250 | 19.183 | −0.796 | 1.00 | 25.10 | C | |
| ATOM | 1700 | N | GLU | A | 218 | 109.145 | 21.398 | 1.213 | 1.00 | 21.92 | N | |
| ATOM | 1701 | CA | GLU | A | 218 | 108.824 | 22.815 | 1.356 | 1.00 | 22.75 | C | |
| ATOM | 1702 | C | GLU | A | 218 | 108.286 | 23.293 | 2.685 | 1.00 | 23.86 | C | |
| ATOM | 1703 | O | GLU | A | 218 | 108.096 | 24.508 | 2.857 | 1.00 | 21.12 | O | |
| ATOM | 1704 | CB | GLU | A | 218 | 107.861 | 23.230 | 0.247 | 1.00 | 25.30 | C | |
| ATOM | 1705 | CG | GLU | A | 218 | 108.483 | 23.034 | −1.127 | 1.00 | 30.46 | C | |
| ATOM | 1706 | CD | GLU | A | 218 | 107.641 | 23.684 | −2.207 | 1.00 | 38.88 | C | |
| ATOM | 1707 | OE1 | GLU | A | 218 | 106.477 | 23.237 | −2.381 | 1.00 | 40.32 | O | |
| ATOM | 1708 | OE2 | GLU | A | 218 | 108.152 | 24.642 | −2.854 | 1.00 | 43.26 | O | |
| ATOM | 1709 | N | THR | A | 219 | 108.077 | 22.376 | 3.635 | 1.00 | 22.78 | N | |
| ATOM | 1710 | CA | THR | A | 219 | 107.552 | 22.767 | 4.925 | 1.00 | 24.31 | C | |
| ATOM | 1711 | C | THR | A | 219 | 108.221 | 22.096 | 6.139 | 1.00 | 24.94 | C | |
| ATOM | 1712 | O | THR | A | 219 | 108.702 | 20.954 | 6.045 | 1.00 | 21.65 | O | |
| ATOM | 1713 | CB | THR | A | 219 | 106.001 | 22.453 | 4.993 | 1.00 | 23.61 | C | |
| ATOM | 1714 | OG1 | THR | A | 219 | 105.803 | 21.054 | 5.024 | 1.00 | 27.40 | O | |
| ATOM | 1715 | CG2 | THR | A | 219 | 105.259 | 22.945 | 3.705 | 1.00 | 29.16 | C | |
| ATOM | 1716 | N | LEU | A | 220 | 108.290 | 22.824 | 7.263 | 1.00 | 24.74 | N | |
| ATOM | 1717 | CA | LEU | A | 220 | 108.758 | 22.246 | 8.536 | 1.00 | 22.94 | C | |
| ATOM | 1718 | C | LEU | A | 220 | 107.507 | 22.380 | 9.406 | 1.00 | 19.99 | C | |
| ATOM | 1719 | O | LEU | A | 220 | 107.091 | 23.503 | 9.705 | 1.00 | 18.54 | O | |
| ATOM | 1720 | CB | LEU | A | 220 | 109.914 | 23.002 | 9.208 | 1.00 | 20.21 | C | |
| ATOM | 1721 | CG | LEU | A | 220 | 110.296 | 22.517 | 10.620 | 1.00 | 22.42 | C | |
| ATOM | 1722 | CD1 | LEU | A | 220 | 110.586 | 21.008 | 10.634 | 1.00 | 24.61 | C | |
| ATOM | 1723 | CD2 | LEU | A | 220 | 111.543 | 23.253 | 11.101 | 1.00 | 24.07 | C | |
| ATOM | 1724 | N | ASP | A | 221 | 106.895 | 21.253 | 9.779 | 1.00 | 19.66 | N | |
| ATOM | 1725 | CA | ASP | A | 221 | 105.652 | 21.330 | 10.570 | 1.00 | 23.35 | C | |
| ATOM | 1726 | C | ASP | A | 221 | 105.826 | 20.897 | 12.028 | 1.00 | 23.88 | C | |
| ATOM | 1727 | O | ASP | A | 221 | 106.385 | 19.822 | 12.316 | 1.00 | 23.57 | O | |
| ATOM | 1728 | CB | ASP | A | 221 | 104.522 | 20.501 | 9.946 | 1.00 | 26.83 | C | |
| ATOM | 1729 | CG | ASP | A | 221 | 104.192 | 20.931 | 8.501 | 1.00 | 38.55 | C | |
| ATOM | 1730 | OD1 | ASP | A | 221 | 104.031 | 22.160 | 8.236 | 1.00 | 31.51 | O | |
| ATOM | 1731 | OD2 | ASP | A | 221 | 104.098 | 20.017 | 7.636 | 1.00 | 42.67 | O | |
| ATOM | 1732 | N | VAL | A | 222 | 105.341 | 21.753 | 12.934 | 1.00 | 21.22 | N | |
| ATOM | 1733 | CA | VAL | A | 222 | 105.459 | 21.533 | 14.371 | 1.00 | 21.26 | C | |
| ATOM | 1734 | C | VAL | A | 222 | 104.008 | 21.318 | 14.854 | 1.00 | 22.51 | C | |
| ATOM | 1735 | O | VAL | A | 222 | 103.205 | 22.240 | 14.854 | 1.00 | 22.46 | O | |
| ATOM | 1736 | CB | VAL | A | 222 | 106.112 | 22.782 | 15.041 | 1.00 | 23.65 | C | |
| ATOM | 1737 | CG1 | VAL | A | 222 | 106.353 | 22.563 | 16.551 | 1.00 | 25.96 | C | |
| ATOM | 1738 | CG2 | VAL | A | 222 | 107.463 | 23.051 | 14.350 | 1.00 | 23.60 | C | |
| ATOM | 1739 | N | GLN | A | 223 | 103.695 | 20.117 | 15.305 | 1.00 | 18.38 | N | |
| ATOM | 1740 | CA | GLN | A | 223 | 102.296 | 19.777 | 15.696 | 1.00 | 17.00 | C | |
| ATOM | 1741 | C | GLN | A | 223 | 102.115 | 19.869 | 17.197 | 1.00 | 18.33 | C | |
| ATOM | 1742 | O | GLN | A | 223 | 102.863 | 19.231 | 17.944 | 1.00 | 17.27 | O | |
| ATOM | 1743 | CB | GLN | A | 223 | 102.029 | 18.364 | 15.166 | 1.00 | 19.34 | C | |
| ATOM | 1744 | CG | GLN | A | 223 | 102.157 | 18.330 | 13.632 | 1.00 | 24.09 | C | |
| ATOM | 1745 | CD | GLN | A | 223 | 102.009 | 16.927 | 13.127 | 1.00 | 30.96 | C | |
| ATOM | 1746 | OE1 | GLN | A | 223 | 102.865 | 16.099 | 13.390 | 1.00 | 33.92 | O | |
| ATOM | 1747 | NE2 | GLN | A | 223 | 100.917 | 16.641 | 12.412 | 1.00 | 33.07 | N | |
| ATOM | 1748 | N | MET | A | 224 | 101.143 | 20.694 | 17.632 | 1.00 | 17.69 | N | |
| ATOM | 1749 | CA | MET | A | 224 | 100.942 | 20.960 | 19.068 | 1.00 | 17.18 | C | |
| ATOM | 1750 | C | MET | A | 224 | 99.566 | 20.469 | 19.553 | 1.00 | 14.46 | C | |
| ATOM | 1751 | O | MET | A | 224 | 98.584 | 20.410 | 18.777 | 1.00 | 15.86 | O | |
| ATOM | 1752 | CB | MET | A | 224 | 101.079 | 22.490 | 19.343 | 1.00 | 12.90 | C | |
| ATOM | 1753 | CG | MET | A | 224 | 102.285 | 23.064 | 18.758 | 1.00 | 15.35 | C | |
| ATOM | 1754 | SD | MET | A | 224 | 102.283 | 24.894 | 18.917 | 1.00 | 17.18 | S | |
| ATOM | 1755 | CE | MET | A | 224 | 104.020 | 25.229 | 18.989 | 1.00 | 20.82 | C | |
| ATOM | 1756 | N | LYS | A | 225 | 99.497 | 20.116 | 20.826 | 1.00 | 15.01 | N | |
| ATOM | 1757 | CA | LYS | A | 225 | 98.254 | 19.615 | 21.420 | 1.00 | 17.20 | C | |
| ATOM | 1758 | C | LYS | A | 225 | 97.079 | 20.577 | 21.075 | 1.00 | 19.43 | C | |
| ATOM | 1759 | O | LYS | A | 225 | 95.996 | 20.145 | 20.578 | 1.00 | 17.01 | O | |
| ATOM | 1760 | CB | LYS | A | 225 | 98.431 | 19.438 | 22.960 | 1.00 | 16.86 | C | |
| ATOM | 1761 | CG | LYS | A | 225 | 97.196 | 18.766 | 23.604 | 1.00 | 18.08 | C | |
| ATOM | 1762 | CD | LYS | A | 225 | 96.975 | 17.321 | 23.091 | 1.00 | 24.51 | C | |
| ATOM | 1763 | CE | LYS | A | 225 | 95.754 | 16.712 | 23.809 | 1.00 | 30.30 | C | |
| ATOM | 1764 | NZ | LYS | A | 225 | 95.411 | 15.310 | 23.395 | 1.00 | 36.28 | N | |
| ATOM | 1765 | N | GLY | A | 226 | 97.302 | 21.867 | 21.332 | 1.00 | 17.33 | N | |
| ATOM | 1766 | CA | GLY | A | 226 | 96.314 | 22.898 | 21.012 | 1.00 | 17.08 | C | |
| ATOM | 1767 | C | GLY | A | 226 | 94.976 | 22.669 | 21.682 | 1.00 | 18.80 | C | |
| ATOM | 1768 | O | GLY | A | 226 | 93.928 | 22.695 | 21.023 | 1.00 | 18.72 | O | |
| ATOM | 1769 | N | GLU | A | 227 | 95.034 | 22.484 | 23.001 | 1.00 | 15.16 | N | |
| ATOM | 1770 | CA | GLU | A | 227 | 93.857 | 22.254 | 23.842 | 1.00 | 14.49 | C | |
| ATOM | 1771 | C | GLU | A | 227 | 93.818 | 23.221 | 25.045 | 1.00 | 14.65 | C | |
| ATOM | 1772 | O | GLU | A | 227 | 94.793 | 23.294 | 25.811 | 1.00 | 17.14 | O | |
| ATOM | 1773 | CB | GLU | A | 227 | 93.903 | 20.797 | 24.383 | 1.00 | 17.76 | C | |
| ATOM | 1774 | CG | GLU | A | 227 | 92.720 | 20.414 | 25.248 | 1.00 | 20.61 | C | |
| ATOM | 1775 | CD | GLU | A | 227 | 92.914 | 19.075 | 25.985 | 1.00 | 24.99 | C | |
| ATOM | 1776 | OE1 | GLU | A | 227 | 93.893 | 18.377 | 25.672 | 1.00 | 27.11 | O | |
| ATOM | 1777 | OE2 | GLU | A | 227 | 92.082 | 18.747 | 26.868 | 1.00 | 29.06 | O | |
| ATOM | 1778 | N | PHE | A | 228 | 92.700 | 23.945 | 25.211 | 1.00 | 16.67 | N | |
| ATOM | 1779 | CA | PHE | A | 228 | 92.520 | 24.739 | 26.414 | 1.00 | 15.56 | C | |
| ATOM | 1780 | C | PHE | A | 228 | 92.250 | 23.724 | 27.563 | 1.00 | 16.34 | C | |
| ATOM | 1781 | O | PHE | A | 228 | 91.569 | 22.691 | 27.348 | 1.00 | 18.01 | O | |
| ATOM | 1782 | CB | PHE | A | 228 | 91.280 | 25.658 | 26.310 | 1.00 | 14.54 | C | |
| ATOM | 1783 | CG | PHE | A | 228 | 91.519 | 26.907 | 25.507 | 1.00 | 14.31 | C | |
| ATOM | 1784 | CD1 | PHE | A | 228 | 91.738 | 26.860 | 24.134 | 1.00 | 15.78 | C | |
| ATOM | 1785 | CD2 | PHE | A | 228 | 91.537 | 28.146 | 26.149 | 1.00 | 17.56 | C | |
| ATOM | 1786 | CE1 | PHE | A | 228 | 91.989 | 28.029 | 23.389 | 1.00 | 17.26 | C | |
| ATOM | 1787 | CE2 | PHE | A | 228 | 91.784 | 29.314 | 25.430 | 1.00 | 16.11 | C | |
| ATOM | 1788 | CZ | PHE | A | 228 | 92.010 | 29.253 | 24.044 | 1.00 | 15.69 | C | |
| ATOM | 1789 | N | TYR | A | 229 | 92.744 | 24.007 | 28.772 | 1.00 | 15.48 | N | |
| ATOM | 1790 | CA | TYR | A | 229 | 92.513 | 23.099 | 29.911 | 1.00 | 19.84 | C | |
| ATOM | 1791 | C | TYR | A | 229 | 92.344 | 23.881 | 31.229 | 1.00 | 20.68 | C | |
| ATOM | 1792 | O | TYR | A | 229 | 92.667 | 25.068 | 31.326 | 1.00 | 18.45 | O | |
| ATOM | 1793 | CB | TYR | A | 229 | 93.709 | 22.120 | 30.051 | 1.00 | 20.47 | C | |
| ATOM | 1794 | CG | TYR | A | 229 | 94.983 | 22.826 | 30.443 | 1.00 | 22.62 | C | |
| ATOM | 1795 | CD1 | TYR | A | 229 | 95.263 | 23.138 | 31.788 | 1.00 | 26.33 | C | |
| ATOM | 1796 | CD2 | TYR | A | 229 | 95.856 | 23.295 | 29.469 | 1.00 | 20.54 | C | |
| ATOM | 1797 | CE1 | TYR | A | 229 | 96.376 | 23.910 | 32.128 | 1.00 | 27.72 | C | |
| ATOM | 1798 | CE2 | TYR | A | 229 | 96.952 | 24.046 | 29.796 | 1.00 | 24.80 | C | |
| ATOM | 1799 | CZ | TYR | A | 229 | 97.218 | 24.364 | 31.115 | 1.00 | 26.20 | C | |
| ATOM | 1800 | OH | TYR | A | 229 | 98.312 | 25.168 | 31.385 | 1.00 | 32.46 | O | |
| ATOM | 1801 | N | SER | A | 230 | 91.796 | 23.197 | 32.226 | 1.00 | 23.78 | N | |
| ATOM | 1802 | CA | SER | A | 230 | 91.611 | 23.750 | 33.579 | 1.00 | 26.93 | C | |
| ATOM | 1803 | C | SER | A | 230 | 92.693 | 23.115 | 34.479 | 1.00 | 31.83 | C | |
| ATOM | 1804 | O | SER | A | 230 | 93.083 | 21.975 | 34.256 | 1.00 | 31.61 | O | |
| ATOM | 1805 | CB | SER | A | 230 | 90.232 | 23.360 | 34.114 | 1.00 | 30.49 | C | |
| ATOM | 1806 | OG | SER | A | 230 | 90.187 | 23.501 | 35.521 | 1.00 | 31.30 | O | |
| ATOM | 1807 | N | GLU | A | 231 | 93.209 | 23.826 | 35.475 | 1.00 | 37.31 | N | |
| ATOM | 1808 | CA | GLU | A | 231 | 94.212 | 23.176 | 36.330 | 1.00 | 44.66 | C | |
| ATOM | 1809 | C | GLU | A | 231 | 93.508 | 22.301 | 37.360 | 1.00 | 48.25 | C | |
| ATOM | 1810 | O | GLU | A | 231 | 94.153 | 21.529 | 38.072 | 1.00 | 50.37 | O | |
| ATOM | 1811 | CB | GLU | A | 231 | 95.140 | 24.197 | 37.003 | 1.00 | 46.75 | C | |
| ATOM | 1812 | CG | GLU | A | 231 | 96.405 | 24.427 | 36.176 | 1.00 | 49.76 | C | |
| ATOM | 1813 | CD | GLU | A | 231 | 97.026 | 25.793 | 36.391 | 1.00 | 51.66 | C | |
| ATOM | 1814 | OE1 | GLU | A | 231 | 97.513 | 26.039 | 37.513 | 1.00 | 51.75 | O | |
| ATOM | 1815 | OE2 | GLU | A | 231 | 97.027 | 26.617 | 35.435 | 1.00 | 54.23 | O | |
| ATOM | 1816 | N | ASN | A | 232 | 92.177 | 22.396 | 37.399 | 1.00 | 51.16 | N | |
| ATOM | 1817 | CA | ASN | A | 232 | 91.366 | 21.597 | 38.316 | 1.00 | 53.91 | C | |
| ATOM | 1818 | C | ASN | A | 232 | 90.819 | 20.338 | 37.636 | 1.00 | 55.63 | C | |
| ATOM | 1819 | O | ASN | A | 232 | 90.823 | 19.260 | 38.228 | 1.00 | 57.18 | O | |
| ATOM | 1820 | CB | ASN | A | 232 | 90.200 | 22.443 | 38.870 | 1.00 | 54.72 | C | |
| ATOM | 1821 | N | HIS | A | 233 | 90.347 | 20.461 | 36.398 | 1.00 | 57.55 | N | |
| ATOM | 1822 | CA | HIS | A | 233 | 89.790 | 19.306 | 35.682 | 1.00 | 58.37 | C | |
| ATOM | 1823 | C | HIS | A | 233 | 90.874 | 18.476 | 34.992 | 1.00 | 58.77 | C | |
| ATOM | 1824 | O | HIS | A | 233 | 92.025 | 18.914 | 34.884 | 1.00 | 60.07 | O | |
| ATOM | 1825 | CB | HIS | A | 233 | 88.745 | 19.769 | 34.651 | 1.00 | 58.42 | C | |
| ATOM | 1826 | N | HIS | A | 234 | 90.507 | 17.281 | 34.519 | 1.00 | 57.54 | N | |
| ATOM | 1827 | CA | HIS | A | 234 | 91.470 | 16.400 | 33.838 | 1.00 | 55.46 | C | |
| ATOM | 1828 | C | HIS | A | 234 | 90.955 | 15.725 | 32.553 | 1.00 | 53.71 | C | |
| ATOM | 1829 | O | HIS | A | 234 | 91.744 | 15.372 | 31.671 | 1.00 | 54.83 | O | |
| ATOM | 1830 | CB | HIS | A | 234 | 91.971 | 15.325 | 34.815 | 1.00 | 56.68 | C | |
| ATOM | 1831 | N | ASN | A | 235 | 89.638 | 15.558 | 32.449 | 1.00 | 49.69 | N | |
| ATOM | 1832 | CA | ASN | A | 235 | 88.992 | 14.897 | 31.304 | 1.00 | 45.13 | C | |
| ATOM | 1833 | C | ASN | A | 235 | 89.284 | 15.462 | 29.901 | 1.00 | 40.67 | C | |
| ATOM | 1834 | O | ASN | A | 235 | 89.236 | 16.671 | 29.692 | 1.00 | 40.43 | O | |
| ATOM | 1835 | CB | ASN | A | 235 | 87.485 | 14.885 | 31.536 | 1.00 | 44.81 | C | |
| ATOM | 1836 | N | PRO | A | 236 | 89.605 | 14.589 | 28.921 | 1.00 | 36.39 | N | |
| ATOM | 1837 | CA | PRO | A | 236 | 89.869 | 15.117 | 27.572 | 1.00 | 33.34 | C | |
| ATOM | 1838 | C | PRO | A | 236 | 88.503 | 15.587 | 27.039 | 1.00 | 28.17 | C | |
| ATOM | 1839 | O | PRO | A | 236 | 87.475 | 15.172 | 27.550 | 1.00 | 27.99 | O | |
| ATOM | 1840 | CB | PRO | A | 236 | 90.346 | 13.886 | 26.789 | 1.00 | 36.52 | C | |
| ATOM | 1841 | CG | PRO | A | 236 | 90.832 | 12.954 | 27.864 | 1.00 | 36.76 | C | |
| ATOM | 1842 | CD | PRO | A | 236 | 89.843 | 13.133 | 28.962 | 1.00 | 38.67 | C | |
| ATOM | 1843 | N | PRO | A | 237 | 88.489 | 16.466 | 26.031 | 1.00 | 23.79 | N | |
| ATOM | 1844 | CA | PRO | A | 237 | 87.231 | 16.950 | 25.443 | 1.00 | 20.12 | C | |
| ATOM | 1845 | C | PRO | A | 237 | 86.593 | 15.697 | 24.803 | 1.00 | 22.36 | C | |
| ATOM | 1846 | O | PRO | A | 237 | 87.294 | 14.776 | 24.430 | 1.00 | 20.34 | O | |
| ATOM | 1847 | CB | PRO | A | 237 | 87.709 | 17.907 | 24.355 | 1.00 | 23.89 | C | |
| ATOM | 1848 | CG | PRO | A | 237 | 89.084 | 18.374 | 24.869 | 1.00 | 28.62 | C | |
| ATOM | 1849 | CD | PRO | A | 237 | 89.652 | 17.077 | 25.386 | 1.00 | 21.45 | C | |
| ATOM | 1850 | N | PRO | A | 238 | 85.280 | 15.660 | 24.681 | 1.00 | 17.79 | N | |
| ATOM | 1851 | CA | PRO | A | 238 | 84.682 | 14.462 | 24.068 | 1.00 | 18.04 | C | |
| ATOM | 1852 | C | PRO | A | 238 | 84.679 | 14.478 | 22.535 | 1.00 | 23.31 | C | |
| ATOM | 1853 | O | PRO | A | 238 | 84.262 | 13.520 | 21.905 | 1.00 | 25.60 | O | |
| ATOM | 1854 | CB | PRO | A | 238 | 83.285 | 14.445 | 24.659 | 1.00 | 21.14 | C | |
| ATOM | 1855 | CG | PRO | A | 238 | 82.969 | 15.974 | 24.820 | 1.00 | 21.29 | C | |
| ATOM | 1856 | CD | PRO | A | 238 | 84.299 | 16.439 | 25.451 | 1.00 | 18.37 | C | |
| ATOM | 1857 | N | PHE | A | 239 | 85.168 | 15.553 | 21.921 | 1.00 | 21.27 | N | |
| ATOM | 1858 | CA | PHE | A | 239 | 85.233 | 15.658 | 20.469 | 1.00 | 21.01 | C | |
| ATOM | 1859 | C | PHE | A | 239 | 86.708 | 15.583 | 20.060 | 1.00 | 21.12 | C | |
| ATOM | 1860 | O | PHE | A | 239 | 87.588 | 15.632 | 20.916 | 1.00 | 20.28 | O | |
| ATOM | 1861 | CB | PHE | A | 239 | 84.549 | 16.958 | 20.031 | 1.00 | 22.95 | C | |
| ATOM | 1862 | CG | PHE | A | 239 | 84.903 | 18.142 | 20.873 | 1.00 | 21.34 | C | |
| ATOM | 1863 | CD1 | PHE | A | 239 | 86.094 | 18.813 | 20.651 | 1.00 | 21.37 | C | |
| ATOM | 1864 | CD2 | PHE | A | 239 | 84.049 | 18.576 | 21.899 | 1.00 | 23.79 | C | |
| ATOM | 1865 | CE1 | PHE | A | 239 | 86.443 | 19.924 | 21.455 | 1.00 | 23.01 | C | |
| ATOM | 1866 | CE2 | PHE | A | 239 | 84.384 | 19.680 | 22.695 | 1.00 | 22.92 | C | |
| ATOM | 1867 | CZ | PHE | A | 239 | 85.585 | 20.351 | 22.468 | 1.00 | 21.34 | C | |
| ATOM | 1868 | N | ALA | A | 240 | 86.978 | 15.479 | 18.759 | 1.00 | 23.40 | N | |
| ATOM | 1869 | CA | ALA | A | 240 | 88.327 | 15.291 | 18.246 | 1.00 | 24.11 | C | |
| ATOM | 1870 | C | ALA | A | 240 | 88.760 | 16.344 | 17.257 | 1.00 | 21.45 | C | |
| ATOM | 1871 | O | ALA | A | 240 | 87.946 | 16.963 | 16.595 | 1.00 | 22.52 | O | |
| ATOM | 1872 | CB | ALA | A | 240 | 88.432 | 13.909 | 17.566 | 1.00 | 28.44 | C | |
| ATOM | 1873 | N | PRO | A | 241 | 90.062 | 16.521 | 17.119 | 1.00 | 21.88 | N | |
| ATOM | 1874 | CA | PRO | A | 241 | 90.578 | 17.516 | 16.180 | 1.00 | 22.65 | C | |
| ATOM | 1875 | C | PRO | A | 241 | 90.315 | 17.063 | 14.731 | 1.00 | 21.80 | C | |
| ATOM | 1876 | O | PRO | A | 241 | 90.270 | 15.821 | 14.444 | 1.00 | 20.85 | O | |
| ATOM | 1877 | CB | PRO | A | 241 | 92.081 | 17.558 | 16.480 | 1.00 | 22.51 | C | |
| ATOM | 1878 | CG | PRO | A | 241 | 92.190 | 16.958 | 17.874 | 1.00 | 25.33 | C | |
| ATOM | 1879 | CD | PRO | A | 241 | 91.129 | 15.945 | 17.961 | 1.00 | 24.33 | C | |
| ATOM | 1880 | N | PRO | A | 242 | 90.147 | 18.022 | 13.808 | 1.00 | 19.29 | N | |
| ATOM | 1881 | CA | PRO | A | 242 | 89.911 | 17.705 | 12.389 | 1.00 | 18.79 | C | |
| ATOM | 1882 | C | PRO | A | 242 | 91.267 | 17.579 | 11.683 | 1.00 | 20.78 | C | |
| ATOM | 1883 | O | PRO | A | 242 | 92.300 | 17.934 | 12.237 | 1.00 | 20.96 | O | |
| ATOM | 1884 | CB | PRO | A | 242 | 89.149 | 18.935 | 11.876 | 1.00 | 19.96 | C | |
| ATOM | 1885 | CG | PRO | A | 242 | 89.924 | 20.079 | 12.624 | 1.00 | 17.80 | C | |
| ATOM | 1886 | CD | PRO | A | 242 | 90.141 | 19.504 | 14.036 | 1.00 | 19.04 | C | |
| ATOM | 1887 | N | AVAL | A | 243 | 91.290 | 17.023 | 10.478 | 0.50 | 22.70 | N | |
| ATOM | 1888 | N | BVAL | A | 243 | 91.262 | 17.057 | 10.464 | 0.50 | 22.06 | N | |
| ATOM | 1889 | CA | AVAL | A | 243 | 92.571 | 16.960 | 9.777 | 0.50 | 21.31 | C | |
| ATOM | 1890 | CA | BVAL | A | 243 | 92.509 | 16.969 | 9.704 | 0.50 | 19.93 | C | |
| ATOM | 1891 | C | AVAL | A | 243 | 92.705 | 18.376 | 9.196 | 0.50 | 21.94 | C | |
| ATOM | 1892 | C | BVAL | A | 243 | 92.700 | 18.381 | 9.132 | 0.50 | 21.31 | C | |
| ATOM | 1893 | O | AVAL | A | 243 | 91.709 | 18.981 | 8.792 | 0.50 | 22.85 | O | |
| ATOM | 1894 | O | BVAL | A | 243 | 91.734 | 18.994 | 8.668 | 0.50 | 22.80 | O | |
| ATOM | 1895 | CB | AVAL | A | 243 | 92.559 | 15.888 | 8.640 | 0.50 | 25.23 | C | |
| ATOM | 1896 | CB | BVAL | A | 243 | 92.376 | 15.897 | 8.572 | 0.50 | 22.72 | C | |
| ATOM | 1897 | CG1 | AVAL | A | 243 | 91.436 | 16.171 | 7.673 | 0.50 | 23.84 | C | |
| ATOM | 1898 | CG1 | BVAL | A | 243 | 93.298 | 16.212 | 7.398 | 0.50 | 21.19 | C | |
| ATOM | 1899 | CG2 | AVAL | A | 243 | 93.919 | 15.855 | 7.930 | 0.50 | 24.59 | C | |
| ATOM | 1900 | CG2 | BVAL | A | 243 | 92.722 | 14.534 | 9.152 | 0.50 | 18.72 | C | |
| ATOM | 1901 | N | MET | A | 244 | 93.913 | 18.923 | 9.204 | 1.00 | 20.72 | N | |
| ATOM | 1902 | CA | MET | A | 244 | 94.139 | 20.254 | 8.675 | 1.00 | 23.41 | C | |
| ATOM | 1903 | C | MET | A | 244 | 95.184 | 20.107 | 7.570 | 1.00 | 26.71 | C | |
| ATOM | 1904 | O | MET | A | 244 | 96.371 | 19.901 | 7.832 | 1.00 | 30.28 | O | |
| ATOM | 1905 | CB | MET | A | 244 | 94.646 | 21.261 | 9.760 | 1.00 | 20.59 | C | |
| ATOM | 1906 | CG | MET | A | 244 | 93.680 | 21.490 | 10.908 | 1.00 | 22.85 | C | |
| ATOM | 1907 | SD | MET | A | 244 | 94.346 | 22.801 | 12.020 | 1.00 | 20.09 | S | |
| ATOM | 1908 | CE | MET | A | 244 | 95.453 | 21.877 | 13.069 | 1.00 | 19.59 | C | |
| ATOM | 1909 | N | GLU | A | 245 | 94.736 | 20.200 | 6.328 | 1.00 | 26.88 | N | |
| ATOM | 1910 | CA | GLU | A | 245 | 95.666 | 20.090 | 5.187 | 1.00 | 31.38 | C | |
| ATOM | 1911 | C | GLU | A | 245 | 95.583 | 21.338 | 4.336 | 1.00 | 31.54 | C | |
| ATOM | 1912 | O | GLU | A | 245 | 94.490 | 21.800 | 3.999 | 1.00 | 31.62 | O | |
| ATOM | 1913 | CB | GLU | A | 245 | 95.333 | 18.863 | 4.327 | 1.00 | 31.53 | C | |
| ATOM | 1914 | CG | GLU | A | 245 | 95.991 | 18.838 | 2.928 | 1.00 | 41.49 | C | |
| ATOM | 1915 | CD | GLU | A | 245 | 95.416 | 19.893 | 1.930 | 1.00 | 47.99 | C | |
| ATOM | 1916 | OE1 | GLU | A | 245 | 94.192 | 20.191 | 2.013 | 1.00 | 52.97 | O | |
| ATOM | 1917 | OE2 | GLU | A | 245 | 96.181 | 20.408 | 1.053 | 1.00 | 51.37 | O | |
| ATOM | 1918 | N | PHE | A | 246 | 96.746 | 21.899 | 4.016 | 1.00 | 33.99 | N | |
| ATOM | 1919 | CA | PHE | A | 246 | 96.823 | 23.078 | 3.164 | 1.00 | 34.20 | C | |
| ATOM | 1920 | C | PHE | A | 246 | 98.127 | 23.070 | 2.373 | 1.00 | 37.48 | C | |
| ATOM | 1921 | O | PHE | A | 246 | 99.170 | 22.605 | 2.849 | 1.00 | 34.14 | O | |
| ATOM | 1922 | CB | PHE | A | 246 | 96.664 | 24.374 | 3.972 | 1.00 | 32.58 | C | |
| ATOM | 1923 | CG | PHE | A | 246 | 97.546 | 24.463 | 5.166 | 1.00 | 29.18 | C | |
| ATOM | 1924 | CD1 | PHE | A | 246 | 97.309 | 23.680 | 6.287 | 1.00 | 28.47 | C | |
| ATOM | 1925 | CD2 | PHE | A | 246 | 98.586 | 25.370 | 5.193 | 1.00 | 28.19 | C | |
| ATOM | 1926 | CE1 | PHE | A | 246 | 98.107 | 23.816 | 7.413 | 1.00 | 28.92 | C | |
| ATOM | 1927 | CE2 | PHE | A | 246 | 99.376 | 25.513 | 6.309 | 1.00 | 26.95 | C | |
| ATOM | 1928 | CZ | PHE | A | 246 | 99.145 | 24.744 | 7.420 | 1.00 | 29.16 | C | |
| ATOM | 1929 | N | PRO | A | 247 | 98.086 | 23.633 | 1.155 | 1.00 | 40.99 | N | |
| ATOM | 1930 | CA | PRO | A | 247 | 99.244 | 23.685 | 0.261 | 1.00 | 40.38 | C | |
| ATOM | 1931 | C | PRO | A | 247 | 100.409 | 24.474 | 0.810 | 1.00 | 39.40 | C | |
| ATOM | 1932 | O | PRO | A | 247 | 100.237 | 25.415 | 1.591 | 1.00 | 39.23 | O | |
| ATOM | 1933 | CB | PRO | A | 247 | 98.657 | 24.311 | −1.002 | 1.00 | 44.55 | C | |
| ATOM | 1934 | CG | PRO | A | 247 | 97.751 | 25.378 | −0.401 | 1.00 | 44.21 | C | |
| ATOM | 1935 | CD | PRO | A | 247 | 97.064 | 24.622 | 0.738 | 1.00 | 43.41 | C | |
| ATOM | 1936 | N | ALA | A | 248 | 101.609 | 24.068 | 0.404 | 1.00 | 38.78 | N | |
| ATOM | 1937 | CA | ALA | A | 248 | 102.800 | 24.785 | 0.809 | 1.00 | 40.12 | C | |
| ATOM | 1938 | C | ALA | A | 248 | 102.703 | 26.132 | 0.064 | 1.00 | 40.68 | C | |
| ATOM | 1939 | O | ALA | A | 248 | 102.349 | 26.190 | −1.123 | 1.00 | 40.32 | O | |
| ATOM | 1940 | CB | ALA | A | 248 | 104.061 | 24.022 | 0.382 | 1.00 | 40.93 | C | |
| ATOM | 1941 | N | ALA | A | 249 | 102.978 | 27.204 | 0.791 | 1.00 | 37.54 | N | |
| ATOM | 1942 | CA | ALA | A | 249 | 102.955 | 28.557 | 0.254 | 1.00 | 38.92 | C | |
| ATOM | 1943 | C | ALA | A | 249 | 104.275 | 29.087 | 0.779 | 1.00 | 37.54 | C | |
| ATOM | 1944 | O | ALA | A | 249 | 104.769 | 28.600 | 1.789 | 1.00 | 35.55 | O | |
| ATOM | 1945 | CB | ALA | A | 249 | 101.785 | 29.341 | 0.822 | 1.00 | 38.19 | C | |
| ATOM | 1946 | N | HIS | A | 250 | 104.846 | 30.067 | 0.089 | 1.00 | 38.81 | N | |
| ATOM | 1947 | CA | HIS | A | 250 | 106.140 | 30.594 | 0.478 | 1.00 | 39.15 | C | |
| ATOM | 1948 | C | HIS | A | 250 | 106.127 | 32.104 | 0.622 | 1.00 | 38.15 | C | |
| ATOM | 1949 | O | HIS | A | 250 | 107.160 | 32.710 | 0.874 | 1.00 | 36.45 | O | |
| ATOM | 1950 | CB | HIS | A | 250 | 107.182 | 30.201 | −0.585 | 1.00 | 43.01 | C | |
| ATOM | 1951 | CG | HIS | A | 250 | 106.868 | 28.920 | −1.298 | 1.00 | 45.37 | C | |
| ATOM | 1952 | ND1 | HIS | A | 250 | 107.034 | 28.769 | −2.658 | 1.00 | 47.21 | N | |
| ATOM | 1953 | CD2 | HIS | A | 250 | 106.403 | 27.730 | −0.844 | 1.00 | 47.20 | C | |
| ATOM | 1954 | CE1 | HIS | A | 250 | 106.682 | 27.546 | −3.011 | 1.00 | 48.82 | C | |
| ATOM | 1955 | NE2 | HIS | A | 250 | 106.294 | 26.893 | −1.929 | 1.00 | 46.58 | N | |
| ATOM | 1956 | N | ASP | A | 251 | 104.968 | 32.714 | 0.434 | 1.00 | 34.36 | N | |
| ATOM | 1957 | CA | ASP | A | 251 | 104.864 | 34.159 | 0.549 | 1.00 | 36.92 | C | |
| ATOM | 1958 | C | ASP | A | 251 | 105.114 | 34.678 | 1.974 | 1.00 | 34.32 | C | |
| ATOM | 1959 | O | ASP | A | 251 | 105.515 | 35.828 | 2.164 | 1.00 | 35.79 | O | |
| ATOM | 1960 | CB | ASP | A | 251 | 103.485 | 34.600 | 0.051 | 1.00 | 41.81 | C | |
| ATOM | 1961 | CG | ASP | A | 251 | 102.354 | 33.675 | 0.532 | 1.00 | 47.98 | C | |
| ATOM | 1962 | OD1 | ASP | A | 251 | 101.710 | 33.936 | 1.584 | 1.00 | 49.24 | O | |
| ATOM | 1963 | OD2 | ASP | A | 251 | 102.110 | 32.655 | −0.156 | 1.00 | 55.44 | O | |
| ATOM | 1964 | N | ARG | A | 252 | 104.839 | 33.862 | 2.982 | 1.00 | 28.81 | N | |
| ATOM | 1965 | CA | ARG | A | 252 | 105.064 | 34.286 | 4.355 | 1.00 | 24.57 | C | |
| ATOM | 1966 | C | ARG | A | 252 | 105.915 | 33.267 | 5.112 | 1.00 | 23.19 | C | |
| ATOM | 1967 | O | ARG | A | 252 | 106.025 | 32.118 | 4.688 | 1.00 | 23.04 | O | |
| ATOM | 1968 | CB | ARG | A | 252 | 103.731 | 34.513 | 5.071 | 1.00 | 28.63 | C | |
| ATOM | 1969 | CG | ARG | A | 252 | 103.008 | 35.737 | 4.447 | 1.00 | 34.58 | C | |
| ATOM | 1970 | CD | ARG | A | 252 | 101.944 | 36.303 | 5.325 | 1.00 | 42.10 | C | |
| ATOM | 1971 | NE | ARG | A | 252 | 101.503 | 37.608 | 4.834 | 1.00 | 45.30 | N | |
| ATOM | 1972 | CZ | ARG | A | 252 | 100.882 | 37.821 | 3.668 | 1.00 | 51.24 | C | |
| ATOM | 1973 | NH1 | ARG | A | 252 | 100.603 | 36.811 | 2.834 | 1.00 | 51.40 | N | |
| ATOM | 1974 | NH2 | ARG | A | 252 | 100.535 | 39.056 | 3.326 | 1.00 | 49.75 | N | |
| ATOM | 1975 | N | MET | A | 253 | 106.482 | 33.713 | 6.228 | 1.00 | 19.98 | N | |
| ATOM | 1976 | CA | MET | A | 253 | 107.406 | 32.876 | 7.044 | 1.00 | 20.40 | C | |
| ATOM | 1977 | C | MET | A | 253 | 106.803 | 31.714 | 7.836 | 1.00 | 21.82 | C | |
| ATOM | 1978 | O | MET | A | 253 | 107.438 | 30.658 | 8.001 | 1.00 | 20.21 | O | |
| ATOM | 1979 | CB | MET | A | 253 | 108.174 | 33.757 | 8.032 | 1.00 | 17.57 | C | |
| ATOM | 1980 | CG | MET | A | 253 | 109.147 | 34.736 | 7.367 | 1.00 | 19.36 | C | |
| ATOM | 1981 | SD | MET | A | 253 | 110.111 | 35.557 | 8.602 | 1.00 | 22.77 | S | |
| ATOM | 1982 | CE | MET | A | 253 | 110.838 | 36.917 | 7.547 | 1.00 | 27.30 | C | |
| ATOM | 1983 | N | VAL | A | 254 | 105.594 | 31.904 | 8.333 | 1.00 | 19.67 | N | |
| ATOM | 1984 | CA | VAL | A | 254 | 104.982 | 30.859 | 9.132 | 1.00 | 23.94 | C | |
| ATOM | 1985 | C | VAL | A | 254 | 103.482 | 30.811 | 8.904 | 1.00 | 22.95 | C | |
| ATOM | 1986 | O | VAL | A | 254 | 102.835 | 31.833 | 8.511 | 1.00 | 22.10 | O | |
| ATOM | 1987 | CB | VAL | A | 254 | 105.260 | 31.025 | 10.685 | 1.00 | 25.52 | C | |
| ATOM | 1988 | CG1 | VAL | A | 254 | 106.762 | 31.004 | 10.998 | 1.00 | 29.22 | C | |
| ATOM | 1989 | CG2 | VAL | A | 254 | 104.646 | 32.336 | 11.212 | 1.00 | 28.41 | C | |
| ATOM | 1990 | N | TYR | A | 255 | 102.929 | 29.605 | 9.081 | 1.00 | 20.57 | N | |
| ATOM | 1991 | CA | TYR | A | 255 | 101.489 | 29.450 | 8.919 | 1.00 | 19.82 | C | |
| ATOM | 1992 | C | TYR | A | 255 | 100.969 | 28.736 | 10.172 | 1.00 | 24.29 | C | |
| ATOM | 1993 | O | TYR | A | 255 | 101.508 | 27.717 | 10.572 | 1.00 | 25.10 | O | |
| ATOM | 1994 | CB | TYR | A | 255 | 101.147 | 28.672 | 7.627 | 1.00 | 23.43 | C | |
| ATOM | 1995 | CG | TYR | A | 255 | 101.544 | 29.400 | 6.361 | 1.00 | 23.86 | C | |
| ATOM | 1996 | CD1 | TYR | A | 255 | 102.829 | 29.283 | 5.833 | 1.00 | 24.68 | C | |
| ATOM | 1997 | CD2 | TYR | A | 255 | 100.656 | 30.251 | 5.732 | 1.00 | 25.84 | C | |
| ATOM | 1998 | CE1 | TYR | A | 255 | 103.214 | 29.985 | 4.714 | 1.00 | 28.52 | C | |
| ATOM | 1999 | CE2 | TYR | A | 255 | 101.038 | 30.977 | 4.593 | 1.00 | 29.21 | C | |
| ATOM | 2000 | CZ | TYR | A | 255 | 102.310 | 30.843 | 4.098 | 1.00 | 29.63 | C | |
| ATOM | 2001 | OH | TYR | A | 255 | 102.695 | 31.623 | 3.039 | 1.00 | 31.12 | O | |
| ATOM | 2002 | N | LEU | A | 256 | 99.975 | 29.337 | 10.825 | 1.00 | 22.20 | N | |
| ATOM | 2003 | CA | LEU | A | 256 | 99.387 | 28.782 | 12.052 | 1.00 | 22.73 | C | |
| ATOM | 2004 | C | LEU | A | 256 | 98.035 | 28.210 | 11.734 | 1.00 | 20.05 | C | |
| ATOM | 2005 | O | LEU | A | 256 | 97.182 | 28.955 | 11.270 | 1.00 | 22.21 | O | |
| ATOM | 2006 | CB | LEU | A | 256 | 99.162 | 29.910 | 13.065 | 1.00 | 29.27 | C | |
| ATOM | 2007 | CG | LEU | A | 256 | 99.602 | 29.643 | 14.485 | 1.00 | 34.91 | C | |
| ATOM | 2008 | CD1 | LEU | A | 256 | 98.807 | 28.537 | 15.124 | 1.00 | 36.90 | C | |
| ATOM | 2009 | CD2 | LEU | A | 256 | 101.065 | 29.288 | 14.427 | 1.00 | 37.63 | C | |
| ATOM | 2010 | N | GLY | A | 257 | 97.833 | 26.909 | 11.961 | 1.00 | 20.18 | N | |
| ATOM | 2011 | CA | GLY | A | 257 | 96.504 | 26.330 | 11.714 | 1.00 | 20.14 | C | |
| ATOM | 2012 | C | GLY | A | 257 | 95.825 | 26.222 | 13.060 | 1.00 | 18.39 | C | |
| ATOM | 2013 | O | GLY | A | 257 | 96.324 | 25.510 | 13.945 | 1.00 | 20.61 | O | |
| ATOM | 2014 | N | LEU | A | 258 | 94.710 | 26.919 | 13.244 | 1.00 | 15.18 | N | |
| ATOM | 2015 | CA | LEU | A | 258 | 93.992 | 26.886 | 14.536 | 1.00 | 15.25 | C | |
| ATOM | 2016 | C | LEU | A | 258 | 92.684 | 26.142 | 14.273 | 1.00 | 15.59 | C | |
| ATOM | 2017 | O | LEU | A | 258 | 91.756 | 26.658 | 13.641 | 1.00 | 16.49 | O | |
| ATOM | 2018 | CB | LEU | A | 258 | 93.665 | 28.308 | 15.074 | 1.00 | 16.51 | C | |
| ATOM | 2019 | CG | LEU | A | 258 | 94.928 | 29.199 | 15.282 | 1.00 | 16.07 | C | |
| ATOM | 2020 | CD1 | LEU | A | 258 | 94.477 | 30.549 | 15.853 | 1.00 | 17.28 | C | |
| ATOM | 2021 | CD2 | LEU | A | 258 | 95.909 | 28.572 | 16.341 | 1.00 | 17.48 | C | |
| ATOM | 2022 | N | SER | A | 259 | 92.622 | 24.917 | 14.779 | 1.00 | 17.23 | N | |
| ATOM | 2023 | CA | SER | A | 259 | 91.455 | 24.104 | 14.545 | 1.00 | 16.74 | C | |
| ATOM | 2024 | C | SER | A | 259 | 90.187 | 24.482 | 15.260 | 1.00 | 16.58 | C | |
| ATOM | 2025 | O | SER | A | 259 | 90.210 | 25.116 | 16.305 | 1.00 | 15.07 | O | |
| ATOM | 2026 | CB | SER | A | 259 | 91.784 | 22.639 | 14.900 | 1.00 | 15.45 | C | |
| ATOM | 2027 | OG | SER | A | 259 | 91.871 | 22.496 | 16.316 | 1.00 | 15.99 | O | |
| ATOM | 2028 | N | ASP | A | 260 | 89.044 | 24.056 | 14.687 | 1.00 | 16.41 | N | |
| ATOM | 2029 | CA | ASP | A | 260 | 87.794 | 24.311 | 15.369 | 1.00 | 16.86 | C | |
| ATOM | 2030 | C | ASP | A | 260 | 87.865 | 23.702 | 16.778 | 1.00 | 20.16 | C | |
| ATOM | 2031 | O | ASP | A | 260 | 87.395 | 24.287 | 17.762 | 1.00 | 18.71 | O | |
| ATOM | 2032 | CB | ASP | A | 260 | 86.573 | 23.791 | 14.560 | 1.00 | 15.50 | C | |
| ATOM | 2033 | CG | ASP | A | 260 | 86.690 | 22.317 | 14.108 | 1.00 | 22.18 | C | |
| ATOM | 2034 | OD1 | ASP | A | 260 | 87.620 | 21.576 | 14.559 | 1.00 | 17.28 | O | |
| ATOM | 2035 | OD2 | ASP | A | 260 | 85.808 | 21.943 | 13.281 | 1.00 | 19.64 | O | |
| ATOM | 2036 | N | TYR | A | 261 | 88.517 | 22.546 | 16.891 | 1.00 | 15.22 | N | |
| ATOM | 2037 | CA | TYR | A | 261 | 88.686 | 21.870 | 18.168 | 1.00 | 15.66 | C | |
| ATOM | 2038 | C | TYR | A | 261 | 89.324 | 22.816 | 19.243 | 1.00 | 16.80 | C | |
| ATOM | 2039 | O | TYR | A | 261 | 88.830 | 22.955 | 20.360 | 1.00 | 16.98 | O | |
| ATOM | 2040 | CB | TYR | A | 261 | 89.570 | 20.644 | 17.874 | 1.00 | 17.87 | C | |
| ATOM | 2041 | CG | TYR | A | 261 | 90.092 | 19.945 | 19.077 | 1.00 | 18.50 | C | |
| ATOM | 2042 | CD1 | TYR | A | 261 | 91.272 | 20.335 | 19.669 | 1.00 | 19.08 | C | |
| ATOM | 2043 | CD2 | TYR | A | 261 | 89.395 | 18.838 | 19.598 | 1.00 | 19.36 | C | |
| ATOM | 2044 | CE1 | TYR | A | 261 | 91.783 | 19.665 | 20.756 | 1.00 | 24.09 | C | |
| ATOM | 2045 | CE2 | TYR | A | 261 | 89.902 | 18.153 | 20.673 | 1.00 | 22.99 | C | |
| ATOM | 2046 | CZ | TYR | A | 261 | 91.091 | 18.574 | 21.244 | 1.00 | 23.38 | C | |
| ATOM | 2047 | OH | TYR | A | 261 | 91.613 | 17.891 | 22.298 | 1.00 | 29.33 | O | |
| ATOM | 2048 | N | PHE | A | 262 | 90.390 | 23.487 | 18.860 | 1.00 | 15.63 | N | |
| ATOM | 2049 | CA | PHE | A | 262 | 91.098 | 24.437 | 19.737 | 1.00 | 14.26 | C | |
| ATOM | 2050 | C | PHE | A | 262 | 90.097 | 25.437 | 20.351 | 1.00 | 14.52 | C | |
| ATOM | 2051 | O | PHE | A | 262 | 89.978 | 25.561 | 21.592 | 1.00 | 15.62 | O | |
| ATOM | 2052 | CB | PHE | A | 262 | 92.153 | 25.130 | 18.870 | 1.00 | 15.43 | C | |
| ATOM | 2053 | CG | PHE | A | 262 | 92.851 | 26.301 | 19.513 | 1.00 | 19.33 | C | |
| ATOM | 2054 | CD1 | PHE | A | 262 | 93.852 | 26.112 | 20.463 | 1.00 | 20.88 | C | |
| ATOM | 2055 | CD2 | PHE | A | 262 | 92.524 | 27.592 | 19.127 | 1.00 | 22.45 | C | |
| ATOM | 2056 | CE1 | PHE | A | 262 | 94.519 | 27.212 | 21.009 | 1.00 | 21.68 | C | |
| ATOM | 2057 | CE2 | PHE | A | 262 | 93.181 | 28.713 | 19.676 | 1.00 | 23.10 | C | |
| ATOM | 2058 | CZ | PHE | A | 262 | 94.169 | 28.531 | 20.605 | 1.00 | 21.28 | C | |
| ATOM | 2059 | N | PHE | A | 263 | 89.309 | 26.075 | 19.484 | 1.00 | 14.45 | N | |
| ATOM | 2060 | CA | PHE | A | 263 | 88.352 | 27.075 | 19.943 | 1.00 | 15.30 | C | |
| ATOM | 2061 | C | PHE | A | 263 | 87.258 | 26.479 | 20.819 | 1.00 | 15.97 | C | |
| ATOM | 2062 | O | PHE | A | 263 | 86.796 | 27.095 | 21.814 | 1.00 | 15.61 | O | |
| ATOM | 2063 | CB | PHE | A | 263 | 87.736 | 27.802 | 18.728 | 1.00 | 17.51 | C | |
| ATOM | 2064 | CG | PHE | A | 263 | 88.724 | 28.629 | 17.955 | 1.00 | 20.10 | C | |
| ATOM | 2065 | CD1 | PHE | A | 263 | 89.242 | 29.796 | 18.504 | 1.00 | 20.71 | C | |
| ATOM | 2066 | CD2 | PHE | A | 263 | 89.151 | 28.230 | 16.688 | 1.00 | 19.83 | C | |
| ATOM | 2067 | CE1 | PHE | A | 263 | 90.209 | 30.565 | 17.792 | 1.00 | 20.61 | C | |
| ATOM | 2068 | CE2 | PHE | A | 263 | 90.087 | 28.977 | 15.969 | 1.00 | 20.83 | C | |
| ATOM | 2069 | CZ | PHE | A | 263 | 90.633 | 30.152 | 16.523 | 1.00 | 22.55 | C | |
| ATOM | 2070 | N | ASN | A | 264 | 86.756 | 25.301 | 20.424 | 1.00 | 15.18 | N | |
| ATOM | 2071 | CA | ASN | A | 264 | 85.726 | 24.675 | 21.214 | 1.00 | 16.06 | C | |
| ATOM | 2072 | C | ASN | A | 264 | 86.223 | 24.205 | 22.606 | 1.00 | 15.79 | C | |
| ATOM | 2073 | O | ASN | A | 264 | 85.421 | 24.152 | 23.550 | 1.00 | 17.13 | O | |
| ATOM | 2074 | CB | ASN | A | 264 | 85.143 | 23.495 | 20.428 | 1.00 | 16.36 | C | |
| ATOM | 2075 | CG | ASN | A | 264 | 84.117 | 23.952 | 19.359 | 1.00 | 21.05 | C | |
| ATOM | 2076 | OD1 | ASN | A | 264 | 82.931 | 24.183 | 19.688 | 1.00 | 26.99 | O | |
| ATOM | 2077 | ND2 | ASN | A | 264 | 84.559 | 24.097 | 18.123 | 1.00 | 21.79 | N | |
| ATOM | 2078 | N | THR | A | 265 | 87.506 | 23.805 | 22.732 | 1.00 | 15.14 | N | |
| ATOM | 2079 | CA | THR | A | 265 | 87.998 | 23.425 | 24.064 | 1.00 | 13.57 | C | |
| ATOM | 2080 | C | THR | A | 265 | 87.962 | 24.670 | 24.980 | 1.00 | 16.07 | C | |
| ATOM | 2081 | O | THR | A | 265 | 87.731 | 24.531 | 26.186 | 1.00 | 13.96 | O | |
| ATOM | 2082 | CB | THR | A | 265 | 89.430 | 22.812 | 24.094 | 1.00 | 14.49 | C | |
| ATOM | 2083 | OG1 | THR | A | 265 | 90.406 | 23.722 | 23.564 | 1.00 | 16.61 | O | |
| ATOM | 2084 | CG2 | THR | A | 265 | 89.459 | 21.512 | 23.271 | 1.00 | 14.99 | C | |
| ATOM | 2085 | N | ALA | A | 266 | 88.158 | 25.853 | 24.410 | 1.00 | 15.69 | N | |
| ATOM | 2086 | CA | ALA | A | 266 | 88.031 | 27.061 | 25.262 | 1.00 | 17.59 | C | |
| ATOM | 2087 | C | ALA | A | 266 | 86.579 | 27.201 | 25.769 | 1.00 | 16.83 | C | |
| ATOM | 2088 | O | ALA | A | 266 | 86.348 | 27.456 | 26.938 | 1.00 | 17.30 | O | |
| ATOM | 2089 | CB | ALA | A | 266 | 88.437 | 28.331 | 24.472 | 1.00 | 16.62 | C | |
| ATOM | 2090 | N | GLY | A | 267 | 85.581 | 27.050 | 24.891 | 1.00 | 16.75 | N | |
| ATOM | 2091 | CA | GLY | A | 267 | 84.215 | 27.136 | 25.401 | 1.00 | 17.52 | C | |
| ATOM | 2092 | C | GLY | A | 267 | 83.926 | 26.095 | 26.483 | 1.00 | 17.67 | C | |
| ATOM | 2093 | O | GLY | A | 267 | 83.226 | 26.356 | 27.438 | 1.00 | 17.14 | O | |
| ATOM | 2094 | N | LEU | A | 268 | 84.447 | 24.880 | 26.337 | 1.00 | 15.09 | N | |
| ATOM | 2095 | CA | LEU | A | 268 | 84.225 | 23.805 | 27.288 | 1.00 | 18.38 | C | |
| ATOM | 2096 | C | LEU | A | 268 | 84.811 | 24.145 | 28.668 | 1.00 | 14.86 | C | |
| ATOM | 2097 | O | LEU | A | 268 | 84.116 | 24.074 | 29.681 | 1.00 | 17.18 | O | |
| ATOM | 2098 | CB | LEU | A | 268 | 84.877 | 22.527 | 26.749 | 1.00 | 20.58 | C | |
| ATOM | 2099 | CG | LEU | A | 268 | 84.867 | 21.351 | 27.719 | 1.00 | 23.31 | C | |
| ATOM | 2100 | CD1 | LEU | A | 268 | 83.438 | 20.902 | 27.876 | 1.00 | 31.11 | C | |
| ATOM | 2101 | CD2 | LEU | A | 268 | 85.716 | 20.178 | 27.132 | 1.00 | 30.53 | C | |
| ATOM | 2102 | N | VAL | A | 269 | 86.064 | 24.590 | 28.705 | 1.00 | 13.96 | N | |
| ATOM | 2103 | CA | VAL | A | 269 | 86.721 | 24.931 | 29.973 | 1.00 | 16.30 | C | |
| ATOM | 2104 | C | VAL | A | 269 | 86.024 | 26.065 | 30.736 | 1.00 | 15.45 | C | |
| ATOM | 2105 | O | VAL | A | 269 | 85.739 | 25.927 | 31.934 | 1.00 | 16.25 | O | |
| ATOM | 2106 | CB | VAL | A | 269 | 88.218 | 25.278 | 29.769 | 1.00 | 14.67 | C | |
| ATOM | 2107 | CG1 | VAL | A | 269 | 88.883 | 25.767 | 31.168 | 1.00 | 17.93 | C | |
| ATOM | 2108 | CG2 | VAL | A | 269 | 88.945 | 24.013 | 29.302 | 1.00 | 17.95 | C | |
| ATOM | 2109 | N | TYR | A | 270 | 85.745 | 27.170 | 30.056 | 1.00 | 15.81 | N | |
| ATOM | 2110 | CA | TYR | A | 270 | 85.103 | 28.293 | 30.755 | 1.00 | 15.28 | C | |
| ATOM | 2111 | C | TYR | A | 270 | 83.656 | 27.950 | 31.179 | 1.00 | 15.51 | C | |
| ATOM | 2112 | O | TYR | A | 270 | 83.226 | 28.314 | 32.260 | 1.00 | 16.48 | O | |
| ATOM | 2113 | CB | TYR | A | 270 | 85.166 | 29.552 | 29.866 | 1.00 | 16.61 | C | |
| ATOM | 2114 | CG | TYR | A | 270 | 86.559 | 30.176 | 29.817 | 1.00 | 16.34 | C | |
| ATOM | 2115 | CD1 | TYR | A | 270 | 87.050 | 30.938 | 30.877 | 1.00 | 18.08 | C | |
| ATOM | 2116 | CD2 | TYR | A | 270 | 87.386 | 29.973 | 28.733 | 1.00 | 18.45 | C | |
| ATOM | 2117 | CE1 | TYR | A | 270 | 88.335 | 31.480 | 30.846 | 1.00 | 18.60 | C | |
| ATOM | 2118 | CE2 | TYR | A | 270 | 88.673 | 30.516 | 28.690 | 1.00 | 20.17 | C | |
| ATOM | 2119 | CZ | TYR | A | 270 | 89.135 | 31.275 | 29.759 | 1.00 | 17.31 | C | |
| ATOM | 2120 | OH | TYR | A | 270 | 90.356 | 31.874 | 29.664 | 1.00 | 17.86 | O | |
| ATOM | 2121 | N | GLN | A | 271 | 82.923 | 27.203 | 30.343 | 1.00 | 14.05 | N | |
| ATOM | 2122 | CA | GLN | A | 271 | 81.569 | 26.878 | 30.766 | 1.00 | 17.26 | C | |
| ATOM | 2123 | C | GLN | A | 271 | 81.626 | 25.950 | 32.008 | 1.00 | 16.43 | C | |
| ATOM | 2124 | O | GLN | A | 271 | 80.846 | 26.094 | 32.956 | 1.00 | 16.56 | O | |
| ATOM | 2125 | CB | GLN | A | 271 | 80.788 | 26.196 | 29.627 | 1.00 | 17.91 | C | |
| ATOM | 2126 | CG | GLN | A | 271 | 79.253 | 26.025 | 29.980 | 1.00 | 19.55 | C | |
| ATOM | 2127 | CD | GLN | A | 271 | 78.987 | 24.694 | 30.596 | 1.00 | 29.59 | C | |
| ATOM | 2128 | OE1 | GLN | A | 271 | 79.722 | 23.744 | 30.335 | 1.00 | 28.56 | O | |
| ATOM | 2129 | NE2 | GLN | A | 271 | 77.925 | 24.594 | 31.401 | 1.00 | 30.36 | N | |
| ATOM | 2130 | N | GLU | A | 272 | 82.529 | 24.966 | 31.973 | 1.00 | 23.33 | N | |
| ATOM | 2131 | CA | GLU | A | 272 | 82.624 | 24.036 | 33.095 | 1.00 | 22.10 | C | |
| ATOM | 2132 | C | GLU | A | 272 | 83.082 | 24.665 | 34.418 | 1.00 | 24.10 | C | |
| ATOM | 2133 | O | GLU | A | 272 | 82.756 | 24.185 | 35.518 | 1.00 | 23.52 | O | |
| ATOM | 2134 | CB | GLU | A | 272 | 83.483 | 22.810 | 32.674 | 1.00 | 23.70 | C | |
| ATOM | 2135 | CG | GLU | A | 272 | 82.778 | 21.966 | 31.641 | 1.00 | 30.03 | C | |
| ATOM | 2136 | CD | GLU | A | 272 | 83.534 | 20.676 | 31.235 | 1.00 | 32.34 | C | |
| ATOM | 2137 | OE1 | GLU | A | 272 | 84.769 | 20.601 | 31.397 | 1.00 | 35.51 | O | |
| ATOM | 2138 | OE2 | GLU | A | 272 | 82.866 | 19.742 | 30.738 | 1.00 | 39.60 | O | |
| ATOM | 2139 | N | ALA | A | 273 | 83.799 | 25.771 | 34.320 | 1.00 | 22.43 | N | |
| ATOM | 2140 | CA | ALA | A | 273 | 84.305 | 26.464 | 35.473 | 1.00 | 20.25 | C | |
| ATOM | 2141 | C | ALA | A | 273 | 83.204 | 27.299 | 36.117 | 1.00 | 20.10 | C | |
| ATOM | 2142 | O | ALA | A | 273 | 83.410 | 27.920 | 37.170 | 1.00 | 21.65 | O | |
| ATOM | 2143 | CB | ALA | A | 273 | 85.432 | 27.353 | 35.057 | 1.00 | 20.47 | C | |
| ATOM | 2144 | N | GLY | A | 274 | 82.073 | 27.383 | 35.432 | 1.00 | 18.17 | N | |
| ATOM | 2145 | CA | GLY | A | 274 | 80.985 | 28.185 | 35.965 | 1.00 | 22.05 | C | |
| ATOM | 2146 | C | GLY | A | 274 | 81.136 | 29.691 | 35.799 | 1.00 | 23.12 | C | |
| ATOM | 2147 | O | GLY | A | 274 | 80.435 | 30.463 | 36.476 | 1.00 | 26.02 | O | |
| ATOM | 2148 | N | VAL | A | 275 | 82.003 | 30.139 | 34.906 | 1.00 | 18.92 | N | |
| ATOM | 2149 | CA | VAL | A | 275 | 82.144 | 31.567 | 34.713 | 1.00 | 20.71 | C | |
| ATOM | 2150 | C | VAL | A | 275 | 81.313 | 32.201 | 33.590 | 1.00 | 18.53 | C | |
| ATOM | 2151 | O | VAL | A | 275 | 81.299 | 33.452 | 33.460 | 1.00 | 20.13 | O | |
| ATOM | 2152 | CB | VAL | A | 275 | 83.609 | 31.974 | 34.508 | 1.00 | 24.08 | C | |
| ATOM | 2153 | CG1 | VAL | A | 275 | 84.404 | 31.660 | 35.783 | 1.00 | 27.69 | C | |
| ATOM | 2154 | CG2 | VAL | A | 275 | 84.185 | 31.300 | 33.308 | 1.00 | 25.66 | C | |
| ATOM | 2155 | N | LEU | A | 276 | 80.620 | 31.384 | 32.792 | 1.00 | 17.52 | N | |
| ATOM | 2156 | CA | LEU | A | 276 | 79.788 | 31.920 | 31.722 | 1.00 | 18.05 | C | |
| ATOM | 2157 | C | LEU | A | 276 | 78.388 | 32.177 | 32.310 | 1.00 | 20.58 | C | |
| ATOM | 2158 | O | LEU | A | 276 | 77.382 | 31.550 | 31.931 | 1.00 | 21.55 | O | |
| ATOM | 2159 | CB | LEU | A | 276 | 79.732 | 30.963 | 30.504 | 1.00 | 19.61 | C | |
| ATOM | 2160 | CG | LEU | A | 276 | 81.054 | 30.614 | 29.806 | 1.00 | 18.97 | C | |
| ATOM | 2161 | CD1 | LEU | A | 276 | 80.838 | 29.830 | 28.507 | 1.00 | 22.04 | C | |
| ATOM | 2162 | CD2 | LEU | A | 276 | 81.790 | 31.973 | 29.491 | 1.00 | 20.31 | C | |
| ATOM | 2163 | N | LYS | A | 277 | 78.335 | 33.093 | 33.271 | 1.00 | 19.75 | N | |
| ATOM | 2164 | CA | LYS | A | 277 | 77.060 | 33.434 | 33.912 | 1.00 | 21.66 | C | |
| ATOM | 2165 | C | LYS | A | 277 | 77.092 | 34.875 | 34.370 | 1.00 | 20.25 | C | |
| ATOM | 2166 | O | LYS | A | 277 | 78.165 | 35.474 | 34.516 | 1.00 | 22.40 | O | |
| ATOM | 2167 | CB | LYS | A | 277 | 76.766 | 32.524 | 35.115 | 1.00 | 24.15 | C | |
| ATOM | 2168 | CG | LYS | A | 277 | 77.620 | 32.832 | 36.364 | 1.00 | 29.16 | C | |
| ATOM | 2169 | CD | LYS | A | 277 | 77.152 | 32.113 | 37.664 | 1.00 | 38.12 | C | |
| ATOM | 2170 | CE | LYS | A | 277 | 76.373 | 30.803 | 37.427 | 1.00 | 42.69 | C | |
| ATOM | 2171 | NZ | LYS | A | 277 | 77.101 | 29.780 | 36.600 | 1.00 | 45.20 | N | |
| ATOM | 2172 | N | MET | A | 278 | 75.920 | 35.441 | 34.584 | 1.00 | 19.99 | N | |
| ATOM | 2173 | CA | MET | A | 278 | 75.828 | 36.817 | 35.037 | 1.00 | 16.84 | C | |
| ATOM | 2174 | C | MET | A | 278 | 74.494 | 37.029 | 35.754 | 1.00 | 18.46 | C | |
| ATOM | 2175 | O | MET | A | 278 | 73.492 | 36.433 | 35.381 | 1.00 | 19.35 | O | |
| ATOM | 2176 | CB | MET | A | 278 | 75.889 | 37.710 | 33.811 | 1.00 | 22.13 | C | |
| ATOM | 2177 | CG | MET | A | 278 | 76.515 | 38.994 | 33.988 | 1.00 | 29.72 | C | |
| ATOM | 2178 | SD | MET | A | 278 | 76.234 | 39.846 | 32.360 | 1.00 | 31.92 | S | |
| ATOM | 2179 | CE | MET | A | 278 | 75.784 | 41.307 | 33.037 | 1.00 | 33.53 | C | |
| ATOM | 2180 | N | THR | A | 279 | 74.500 | 37.834 | 36.809 | 1.00 | 18.01 | N | |
| ATOM | 2181 | CA | THR | A | 279 | 73.257 | 38.158 | 37.527 | 1.00 | 19.46 | C | |
| ATOM | 2182 | C | THR | A | 279 | 72.946 | 39.632 | 37.230 | 1.00 | 22.45 | C | |
| ATOM | 2183 | O | THR | A | 279 | 73.806 | 40.498 | 37.377 | 1.00 | 22.57 | O | |
| ATOM | 2184 | CB | THR | A | 279 | 73.433 | 37.965 | 39.064 | 1.00 | 19.32 | C | |
| ATOM | 2185 | OG1 | THR | A | 279 | 73.552 | 36.562 | 39.360 | 1.00 | 18.84 | O | |
| ATOM | 2186 | CG2 | THR | A | 279 | 72.195 | 38.507 | 39.817 | 1.00 | 23.38 | C | |
| ATOM | 2187 | N | LEU | A | 280 | 71.729 | 39.891 | 36.777 | 1.00 | 22.38 | N | |
| ATOM | 2188 | CA | LEU | A | 280 | 71.286 | 41.249 | 36.475 | 1.00 | 24.48 | C | |
| ATOM | 2189 | C | LEU | A | 280 | 70.288 | 41.699 | 37.548 | 1.00 | 25.89 | C | |
| ATOM | 2190 | O | LEU | A | 280 | 69.339 | 40.971 | 37.899 | 1.00 | 24.93 | O | |
| ATOM | 2191 | CB | LEU | A | 280 | 70.619 | 41.253 | 35.104 | 1.00 | 25.82 | C | |
| ATOM | 2192 | CG | LEU | A | 280 | 71.532 | 40.708 | 33.991 | 1.00 | 32.45 | C | |
| ATOM | 2193 | CD1 | LEU | A | 280 | 70.721 | 40.369 | 32.752 | 1.00 | 33.01 | C | |
| ATOM | 2194 | CD2 | LEU | A | 280 | 72.586 | 41.731 | 33.657 | 1.00 | 32.87 | C | |
| ATOM | 2195 | N | ARG | A | 281 | 70.518 | 42.884 | 38.102 | 1.00 | 27.91 | N | |
| ATOM | 2196 | CA | ARG | A | 281 | 69.635 | 43.481 | 39.125 | 1.00 | 30.19 | C | |
| ATOM | 2197 | C | ARG | A | 281 | 69.163 | 44.814 | 38.534 | 1.00 | 32.36 | C | |
| ATOM | 2198 | O | ARG | A | 281 | 69.808 | 45.344 | 37.607 | 1.00 | 30.59 | O | |
| ATOM | 2199 | CB | ARG | A | 281 | 70.406 | 43.759 | 40.417 | 1.00 | 32.39 | C | |
| ATOM | 2200 | CG | ARG | A | 281 | 70.982 | 42.493 | 41.094 | 1.00 | 38.37 | C | |
| ATOM | 2201 | CD | ARG | A | 281 | 72.179 | 42.862 | 42.001 | 1.00 | 47.28 | C | |
| ATOM | 2202 | NE | ARG | A | 281 | 73.391 | 42.074 | 41.709 | 1.00 | 52.64 | N | |
| ATOM | 2203 | CZ | ARG | A | 281 | 73.608 | 40.824 | 42.134 | 1.00 | 57.96 | C | |
| ATOM | 2204 | NH1 | ARG | A | 281 | 72.694 | 40.199 | 42.880 | 1.00 | 58.25 | N | |
| ATOM | 2205 | NH2 | ARG | A | 281 | 74.746 | 40.191 | 41.830 | 1.00 | 58.24 | N | |
| ATOM | 2206 | N | ASP | A | 282 | 68.049 | 45.330 | 39.061 | 1.00 | 31.98 | N | |
| ATOM | 2207 | CA | ASP | A | 282 | 67.487 | 46.593 | 38.563 | 1.00 | 33.01 | C | |
| ATOM | 2208 | C | ASP | A | 282 | 68.483 | 47.751 | 38.625 | 1.00 | 30.97 | C | |
| ATOM | 2209 | O | ASP | A | 282 | 68.498 | 48.605 | 37.743 | 1.00 | 31.89 | O | |
| ATOM | 2210 | CB | ASP | A | 282 | 66.212 | 46.952 | 39.337 | 1.00 | 33.48 | C | |
| ATOM | 2211 | CG | ASP | A | 282 | 65.386 | 48.041 | 38.641 | 1.00 | 37.38 | C | |
| ATOM | 2212 | OD1 | ASP | A | 282 | 64.802 | 48.880 | 39.339 | 1.00 | 43.48 | O | |
| ATOM | 2213 | OD2 | ASP | A | 282 | 65.300 | 48.070 | 37.409 | 1.00 | 33.42 | O | |
| ATOM | 2214 | N | ASP | A | 283 | 69.343 | 47.782 | 39.633 | 1.00 | 33.43 | N | |
| ATOM | 2215 | CA | ASP | A | 283 | 70.325 | 48.862 | 39.728 | 1.00 | 35.26 | C | |
| ATOM | 2216 | C | ASP | A | 283 | 71.338 | 48.876 | 38.559 | 1.00 | 35.13 | C | |
| ATOM | 2217 | O | ASP | A | 283 | 72.030 | 49.867 | 38.350 | 1.00 | 35.69 | O | |
| ATOM | 2218 | CB | ASP | A | 283 | 71.082 | 48.765 | 41.068 | 1.00 | 40.19 | C | |
| ATOM | 2219 | CG | ASP | A | 283 | 71.859 | 47.455 | 41.209 | 1.00 | 43.35 | C | |
| ATOM | 2220 | OD1 | ASP | A | 283 | 72.989 | 47.360 | 40.657 | 1.00 | 46.39 | O | |
| ATOM | 2221 | OD2 | ASP | A | 283 | 71.335 | 46.515 | 41.855 | 1.00 | 45.72 | O | |
| ATOM | 2222 | N | MET | A | 284 | 71.433 | 47.773 | 37.802 | 1.00 | 29.89 | N | |
| ATOM | 2223 | CA | MET | A | 284 | 72.361 | 47.675 | 36.677 | 1.00 | 25.19 | C | |
| ATOM | 2224 | C | MET | A | 284 | 71.706 | 48.060 | 35.345 | 1.00 | 27.52 | C | |
| ATOM | 2225 | O | MET | A | 284 | 72.383 | 48.136 | 34.313 | 1.00 | 28.22 | O | |
| ATOM | 2226 | CB | MET | A | 284 | 72.915 | 46.227 | 36.590 | 1.00 | 27.31 | C | |
| ATOM | 2227 | CG | MET | A | 284 | 73.696 | 45.754 | 37.833 | 1.00 | 25.10 | C | |
| ATOM | 2228 | SD | MET | A | 284 | 73.835 | 43.893 | 37.967 | 1.00 | 29.95 | S | |
| ATOM | 2229 | CE | MET | A | 284 | 74.694 | 43.573 | 36.464 | 1.00 | 27.19 | C | |
| ATOM | 2230 | N | ILE | A | 285 | 70.399 | 48.276 | 35.357 | 1.00 | 28.04 | N | |
| ATOM | 2231 | CA | ILE | A | 285 | 69.669 | 48.630 | 34.133 | 1.00 | 30.56 | C | |
| ATOM | 2232 | C | ILE | A | 285 | 69.751 | 50.134 | 33.907 | 1.00 | 32.39 | C | |
| ATOM | 2233 | O | ILE | A | 285 | 69.097 | 50.870 | 34.633 | 1.00 | 31.53 | O | |
| ATOM | 2234 | CB | ILE | A | 285 | 68.180 | 48.281 | 34.275 | 1.00 | 32.03 | C | |
| ATOM | 2235 | CG1 | ILE | A | 285 | 68.052 | 46.817 | 34.704 | 1.00 | 32.08 | C | |
| ATOM | 2236 | CG2 | ILE | A | 285 | 67.433 | 48.580 | 32.971 | 1.00 | 30.35 | C | |
| ATOM | 2237 | CD1 | ILE | A | 285 | 68.779 | 45.870 | 33.755 | 1.00 | 35.58 | C | |
| ATOM | 2238 | N | PRO | A | 286 | 70.546 | 50.581 | 32.900 | 1.00 | 32.61 | N | |
| ATOM | 2239 | CA | PRO | A | 286 | 70.764 | 51.989 | 32.511 | 1.00 | 36.58 | C | |
| ATOM | 2240 | C | PRO | A | 286 | 69.516 | 52.833 | 32.666 | 1.00 | 34.52 | C | |
| ATOM | 2241 | O | PRO | A | 286 | 68.403 | 52.407 | 32.376 | 1.00 | 36.47 | O | |
| ATOM | 2242 | CB | PRO | A | 286 | 71.215 | 51.879 | 31.057 | 1.00 | 35.24 | C | |
| ATOM | 2243 | CG | PRO | A | 286 | 72.050 | 50.634 | 31.102 | 1.00 | 37.40 | C | |
| ATOM | 2244 | CD | PRO | A | 286 | 71.214 | 49.683 | 31.938 | 1.00 | 33.96 | C | |
| ATOM | 2245 | N | LYS | A | 287 | 69.720 | 54.048 | 33.151 | 1.00 | 38.14 | N | |
| ATOM | 2246 | CA | LYS | A | 287 | 68.632 | 54.972 | 33.380 | 1.00 | 38.21 | C | |
| ATOM | 2247 | C | LYS | A | 287 | 67.852 | 55.181 | 32.090 | 1.00 | 37.23 | C | |
| ATOM | 2248 | O | LYS | A | 287 | 66.639 | 55.388 | 32.106 | 1.00 | 34.13 | O | |
| ATOM | 2249 | CB | LYS | A | 287 | 69.221 | 56.294 | 33.861 | 1.00 | 39.11 | C | |
| ATOM | 2250 | CG | LYS | A | 287 | 68.227 | 57.406 | 34.022 | 1.00 | 39.87 | C | |
| ATOM | 2251 | CD | LYS | A | 287 | 68.524 | 58.132 | 35.311 | 1.00 | 39.55 | C | |
| ATOM | 2252 | CE | LYS | A | 287 | 69.999 | 58.430 | 35.426 | 1.00 | 36.70 | C | |
| ATOM | 2253 | NZ | LYS | A | 287 | 70.369 | 58.988 | 36.755 | 1.00 | 40.36 | N | |
| ATOM | 2254 | N | GLU | A | 288 | 68.568 | 55.127 | 30.969 | 1.00 | 38.24 | N | |
| ATOM | 2255 | CA | GLU | A | 288 | 67.934 | 55.320 | 29.669 | 1.00 | 39.72 | C | |
| ATOM | 2256 | C | GLU | A | 288 | 66.892 | 54.254 | 29.330 | 1.00 | 41.08 | C | |
| ATOM | 2257 | O | GLU | A | 288 | 66.067 | 54.440 | 28.428 | 1.00 | 39.85 | O | |
| ATOM | 2258 | CB | GLU | A | 288 | 68.979 | 55.358 | 28.560 | 1.00 | 39.55 | C | |
| ATOM | 2259 | CG | GLU | A | 288 | 69.860 | 54.122 | 28.456 | 1.00 | 42.79 | C | |
| ATOM | 2260 | CD | GLU | A | 288 | 70.293 | 53.835 | 27.010 | 1.00 | 44.13 | C | |
| ATOM | 2261 | OE1 | GLU | A | 288 | 70.417 | 54.807 | 26.217 | 1.00 | 44.17 | O | |
| ATOM | 2262 | OE2 | GLU | A | 288 | 70.520 | 52.647 | 26.675 | 1.00 | 42.82 | O | |
| ATOM | 2263 | N | SER | A | 289 | 66.878 | 53.148 | 30.065 | 1.00 | 41.55 | N | |
| ATOM | 2264 | CA | SER | A | 289 | 65.919 | 52.116 | 29.701 | 1.00 | 41.57 | C | |
| ATOM | 2265 | C | SER | A | 289 | 64.537 | 52.195 | 30.327 | 1.00 | 40.41 | C | |
| ATOM | 2266 | O | SER | A | 289 | 64.383 | 52.510 | 31.507 | 1.00 | 41.76 | O | |
| ATOM | 2267 | CB | SER | A | 289 | 66.487 | 50.738 | 30.004 | 1.00 | 41.20 | C | |
| ATOM | 2268 | OG | SER | A | 289 | 65.494 | 49.782 | 29.681 | 1.00 | 46.03 | O | |
| ATOM | 2269 | N | LYS | A | 290 | 63.528 | 51.861 | 29.534 | 1.00 | 38.86 | N | |
| ATOM | 2270 | CA | LYS | A | 290 | 62.168 | 51.848 | 30.023 | 1.00 | 40.53 | C | |
| ATOM | 2271 | C | LYS | A | 290 | 61.911 | 50.548 | 30.776 | 1.00 | 40.08 | C | |
| ATOM | 2272 | O | LYS | A | 290 | 61.037 | 50.463 | 31.613 | 1.00 | 41.53 | O | |
| ATOM | 2273 | CB | LYS | A | 290 | 61.195 | 51.962 | 28.851 | 1.00 | 43.06 | C | |
| ATOM | 2274 | CG | LYS | A | 290 | 61.805 | 51.501 | 27.536 | 1.00 | 45.88 | C | |
| ATOM | 2275 | CD | LYS | A | 290 | 61.158 | 52.189 | 26.336 | 1.00 | 48.41 | C | |
| ATOM | 2276 | CE | LYS | A | 290 | 62.150 | 52.263 | 25.202 | 1.00 | 51.28 | C | |
| ATOM | 2277 | NZ | LYS | A | 290 | 63.444 | 52.807 | 25.733 | 1.00 | 53.00 | N | |
| ATOM | 2278 | N | PHE | A | 291 | 62.695 | 49.533 | 30.474 | 1.00 | 39.87 | N | |
| ATOM | 2279 | CA | PHE | A | 291 | 62.528 | 48.234 | 31.126 | 1.00 | 40.45 | C | |
| ATOM | 2280 | C | PHE | A | 291 | 63.114 | 48.353 | 32.540 | 1.00 | 39.12 | C | |
| ATOM | 2281 | O | PHE | A | 291 | 64.250 | 48.790 | 32.685 | 1.00 | 41.30 | O | |
| ATOM | 2282 | CB | PHE | A | 291 | 63.271 | 47.159 | 30.296 | 1.00 | 39.31 | C | |
| ATOM | 2283 | CG | PHE | A | 291 | 62.900 | 47.162 | 28.832 | 1.00 | 38.38 | C | |
| ATOM | 2284 | CD1 | PHE | A | 291 | 61.730 | 46.568 | 28.396 | 1.00 | 40.75 | C | |
| ATOM | 2285 | CD2 | PHE | A | 291 | 63.694 | 47.822 | 27.893 | 1.00 | 41.33 | C | |
| ATOM | 2286 | CE1 | PHE | A | 291 | 61.352 | 46.634 | 27.054 | 1.00 | 41.56 | C | |
| ATOM | 2287 | CE2 | PHE | A | 291 | 63.314 | 47.889 | 26.548 | 1.00 | 38.64 | C | |
| ATOM | 2288 | CZ | PHE | A | 291 | 62.150 | 47.299 | 26.137 | 1.00 | 40.41 | C | |
| ATOM | 2289 | N | ARG | A | 292 | 62.337 | 48.027 | 33.579 | 1.00 | 34.25 | N | |
| ATOM | 2290 | CA | ARG | A | 292 | 62.883 | 48.073 | 34.943 | 1.00 | 34.51 | C | |
| ATOM | 2291 | C | ARG | A | 292 | 62.738 | 46.671 | 35.515 | 1.00 | 32.77 | C | |
| ATOM | 2292 | O | ARG | A | 292 | 61.751 | 46.004 | 35.246 | 1.00 | 29.04 | O | |
| ATOM | 2293 | CB | ARG | A | 292 | 62.096 | 49.026 | 35.849 | 1.00 | 36.30 | C | |
| ATOM | 2294 | CG | ARG | A | 292 | 61.893 | 50.440 | 35.297 | 1.00 | 39.49 | C | |
| ATOM | 2295 | CD | ARG | A | 292 | 63.145 | 50.978 | 34.656 | 1.00 | 41.52 | C | |
| ATOM | 2296 | NE | ARG | A | 292 | 64.305 | 50.809 | 35.506 | 1.00 | 41.74 | N | |
| ATOM | 2297 | CZ | ARG | A | 292 | 65.539 | 51.113 | 35.132 | 1.00 | 43.04 | C | |
| ATOM | 2298 | NH1 | ARG | A | 292 | 65.769 | 51.613 | 33.906 | 1.00 | 41.27 | N | |
| ATOM | 2299 | NH2 | ARG | A | 292 | 66.543 | 50.899 | 35.979 | 1.00 | 41.81 | N | |
| ATOM | 2300 | N | LEU | A | 293 | 63.711 | 46.221 | 36.296 | 1.00 | 30.32 | N | |
| ATOM | 2301 | CA | LEU | A | 293 | 63.591 | 44.880 | 36.911 | 1.00 | 31.40 | C | |
| ATOM | 2302 | C | LEU | A | 293 | 62.781 | 44.912 | 38.228 | 1.00 | 30.83 | C | |
| ATOM | 2303 | O | LEU | A | 293 | 63.338 | 44.811 | 39.333 | 1.00 | 29.32 | O | |
| ATOM | 2304 | CB | LEU | A | 293 | 64.994 | 44.310 | 37.170 | 1.00 | 30.23 | C | |
| ATOM | 2305 | CG | LEU | A | 293 | 65.784 | 44.057 | 35.890 | 1.00 | 30.94 | C | |
| ATOM | 2306 | CD1 | LEU | A | 293 | 67.160 | 43.389 | 36.198 | 1.00 | 32.35 | C | |
| ATOM | 2307 | CD2 | LEU | A | 293 | 64.971 | 43.136 | 35.010 | 1.00 | 32.47 | C | |
| ATOM | 2308 | N | THR | A | 294 | 61.470 | 45.092 | 38.101 | 1.00 | 31.22 | N | |
| ATOM | 2309 | CA | THR | A | 294 | 60.581 | 45.135 | 39.271 | 1.00 | 30.79 | C | |
| ATOM | 2310 | C | THR | A | 294 | 59.316 | 44.406 | 38.859 | 1.00 | 27.38 | C | |
| ATOM | 2311 | O | THR | A | 294 | 58.986 | 44.380 | 37.658 | 1.00 | 27.61 | O | |
| ATOM | 2312 | CB | THR | A | 294 | 60.217 | 46.605 | 39.633 | 1.00 | 33.98 | C | |
| ATOM | 2313 | OG1 | THR | A | 294 | 59.428 | 47.156 | 38.568 | 1.00 | 35.88 | O | |
| ATOM | 2314 | CG2 | THR | A | 294 | 61.496 | 47.454 | 39.803 | 1.00 | 32.33 | C | |
| ATOM | 2315 | N | THR | A | 295 | 58.597 | 43.830 | 39.822 | 1.00 | 29.10 | N | |
| ATOM | 2316 | CA | THR | A | 295 | 57.357 | 43.121 | 39.505 | 1.00 | 30.44 | C | |
| ATOM | 2317 | C | THR | A | 295 | 56.308 | 44.177 | 39.053 | 1.00 | 32.74 | C | |
| ATOM | 2318 | O | THR | A | 295 | 55.459 | 43.892 | 38.215 | 1.00 | 31.08 | O | |
| ATOM | 2319 | CB | THR | A | 295 | 56.795 | 42.304 | 40.723 | 1.00 | 33.75 | C | |
| ATOM | 2320 | OG1 | THR | A | 295 | 56.522 | 43.176 | 41.829 | 1.00 | 34.41 | O | |
| ATOM | 2321 | CG2 | THR | A | 295 | 57.810 | 41.213 | 41.166 | 1.00 | 27.88 | C | |
| ATOM | 2322 | N | LYS | A | 296 | 56.420 | 45.388 | 39.602 | 1.00 | 34.57 | N | |
| ATOM | 2323 | CA | LYS | A | 296 | 55.511 | 46.472 | 39.217 | 1.00 | 36.19 | C | |
| ATOM | 2324 | C | LYS | A | 296 | 55.614 | 46.625 | 37.698 | 1.00 | 34.75 | C | |
| ATOM | 2325 | O | LYS | A | 296 | 54.624 | 46.435 | 36.969 | 1.00 | 32.70 | O | |
| ATOM | 2326 | CB | LYS | A | 296 | 55.903 | 47.779 | 39.920 | 1.00 | 37.02 | C | |
| ATOM | 2327 | CG | LYS | A | 296 | 54.909 | 48.960 | 39.702 | 1.00 | 42.72 | C | |
| ATOM | 2328 | CD | LYS | A | 296 | 55.120 | 50.043 | 40.770 | 1.00 | 41.86 | C | |
| ATOM | 2329 | CE | LYS | A | 296 | 54.045 | 51.122 | 40.725 | 1.00 | 48.04 | C | |
| ATOM | 2330 | NZ | LYS | A | 296 | 54.202 | 52.124 | 41.847 | 1.00 | 48.53 | N | |
| ATOM | 2331 | N | PHE | A | 297 | 56.824 | 46.899 | 37.208 | 1.00 | 35.27 | N | |
| ATOM | 2332 | CA | PHE | A | 297 | 57.005 | 47.089 | 35.774 | 1.00 | 34.42 | C | |
| ATOM | 2333 | C | PHE | A | 297 | 56.651 | 45.856 | 34.976 | 1.00 | 35.51 | C | |
| ATOM | 2334 | O | PHE | A | 297 | 55.927 | 45.953 | 33.992 | 1.00 | 36.39 | O | |
| ATOM | 2335 | CB | PHE | A | 297 | 58.421 | 47.529 | 35.412 | 1.00 | 37.81 | C | |
| ATOM | 2336 | CG | PHE | A | 297 | 58.615 | 47.706 | 33.923 | 1.00 | 37.97 | C | |
| ATOM | 2337 | CD1 | PHE | A | 297 | 58.031 | 48.781 | 33.253 | 1.00 | 40.65 | C | |
| ATOM | 2338 | CD2 | PHE | A | 297 | 59.297 | 46.739 | 33.171 | 1.00 | 39.24 | C | |
| ATOM | 2339 | CE1 | PHE | A | 297 | 58.110 | 48.894 | 31.856 | 1.00 | 38.51 | C | |
| ATOM | 2340 | CE2 | PHE | A | 297 | 59.376 | 46.846 | 31.777 | 1.00 | 38.45 | C | |
| ATOM | 2341 | CZ | PHE | A | 297 | 58.771 | 47.937 | 31.123 | 1.00 | 39.10 | C | |
| ATOM | 2342 | N | PHE | A | 298 | 57.158 | 44.685 | 35.367 | 1.00 | 33.02 | N | |
| ATOM | 2343 | CA | PHE | A | 298 | 56.796 | 43.491 | 34.614 | 1.00 | 30.06 | C | |
| ATOM | 2344 | C | PHE | A | 298 | 55.286 | 43.279 | 34.572 | 1.00 | 33.42 | C | |
| ATOM | 2345 | O | PHE | A | 298 | 54.775 | 42.685 | 33.624 | 1.00 | 34.10 | O | |
| ATOM | 2346 | CB | PHE | A | 298 | 57.445 | 42.234 | 35.221 | 1.00 | 28.14 | C | |
| ATOM | 2347 | CG | PHE | A | 298 | 58.826 | 41.972 | 34.703 | 1.00 | 29.92 | C | |
| ATOM | 2348 | CD1 | PHE | A | 298 | 59.769 | 43.013 | 34.672 | 1.00 | 29.49 | C | |
| ATOM | 2349 | CD2 | PHE | A | 298 | 59.197 | 40.694 | 34.274 | 1.00 | 32.76 | C | |
| ATOM | 2350 | CE1 | PHE | A | 298 | 61.081 | 42.789 | 34.224 | 1.00 | 29.96 | C | |
| ATOM | 2351 | CE2 | PHE | A | 298 | 60.504 | 40.451 | 33.822 | 1.00 | 32.76 | C | |
| ATOM | 2352 | CZ | PHE | A | 298 | 61.447 | 41.491 | 33.794 | 1.00 | 30.54 | C | |
| ATOM | 2353 | N | GLY | A | 299 | 54.589 | 43.726 | 35.617 | 1.00 | 36.32 | N | |
| ATOM | 2354 | CA | GLY | A | 299 | 53.139 | 43.590 | 35.677 | 1.00 | 40.46 | C | |
| ATOM | 2355 | C | GLY | A | 299 | 52.391 | 44.265 | 34.531 | 1.00 | 42.69 | C | |
| ATOM | 2356 | O | GLY | A | 299 | 51.268 | 43.879 | 34.195 | 1.00 | 43.32 | O | |
| ATOM | 2357 | N | THR | A | 300 | 53.014 | 45.274 | 33.933 | 1.00 | 43.13 | N | |
| ATOM | 2358 | CA | THR | A | 300 | 52.415 | 45.989 | 32.822 | 1.00 | 43.17 | C | |
| ATOM | 2359 | C | THR | A | 300 | 52.311 | 45.084 | 31.611 | 1.00 | 44.09 | C | |
| ATOM | 2360 | O | THR | A | 300 | 51.455 | 45.307 | 30.759 | 1.00 | 43.97 | O | |
| ATOM | 2361 | CB | THR | A | 300 | 53.238 | 47.222 | 32.410 | 1.00 | 43.86 | C | |
| ATOM | 2362 | OG1 | THR | A | 300 | 54.479 | 46.802 | 31.821 | 1.00 | 44.49 | O | |
| ATOM | 2363 | CG2 | THR | A | 300 | 53.513 | 48.108 | 33.609 | 1.00 | 42.85 | C | |
| ATOM | 2364 | N | PHE | A | 301 | 53.159 | 44.058 | 31.506 | 1.00 | 42.79 | N | |
| ATOM | 2365 | CA | PHE | A | 301 | 53.034 | 43.191 | 30.345 | 1.00 | 42.82 | C | |
| ATOM | 2366 | C | PHE | A | 301 | 52.754 | 41.698 | 30.552 | 1.00 | 45.27 | C | |
| ATOM | 2367 | O | PHE | A | 301 | 52.120 | 41.069 | 29.705 | 1.00 | 45.34 | O | |
| ATOM | 2368 | CB | PHE | A | 301 | 54.228 | 43.388 | 29.386 | 1.00 | 42.83 | C | |
| ATOM | 2369 | CG | PHE | A | 301 | 55.563 | 42.994 | 29.950 | 1.00 | 39.71 | C | |
| ATOM | 2370 | CD1 | PHE | A | 301 | 56.275 | 43.865 | 30.769 | 1.00 | 40.02 | C | |
| ATOM | 2371 | CD2 | PHE | A | 301 | 56.138 | 41.780 | 29.606 | 1.00 | 40.65 | C | |
| ATOM | 2372 | CE1 | PHE | A | 301 | 57.544 | 43.544 | 31.236 | 1.00 | 39.74 | C | |
| ATOM | 2373 | CE2 | PHE | A | 301 | 57.419 | 41.443 | 30.070 | 1.00 | 38.92 | C | |
| ATOM | 2374 | CZ | PHE | A | 301 | 58.122 | 42.324 | 30.884 | 1.00 | 40.54 | C | |
| ATOM | 2375 | N | LEU | A | 302 | 53.187 | 41.119 | 31.665 | 1.00 | 46.34 | N | |
| ATOM | 2376 | CA | LEU | A | 302 | 52.933 | 39.694 | 31.886 | 1.00 | 47.31 | C | |
| ATOM | 2377 | C | LEU | A | 302 | 51.795 | 39.488 | 32.884 | 1.00 | 49.46 | C | |
| ATOM | 2378 | O | LEU | A | 302 | 51.427 | 40.409 | 33.596 | 1.00 | 48.56 | O | |
| ATOM | 2379 | CB | LEU | A | 302 | 54.196 | 38.984 | 32.379 | 1.00 | 46.24 | C | |
| ATOM | 2380 | CG | LEU | A | 302 | 55.317 | 38.816 | 31.354 | 1.00 | 46.01 | C | |
| ATOM | 2381 | CD1 | LEU | A | 302 | 56.565 | 38.385 | 32.073 | 1.00 | 45.05 | C | |
| ATOM | 2382 | CD2 | LEU | A | 302 | 54.935 | 37.816 | 30.293 | 1.00 | 44.52 | C | |
| ATOM | 2383 | N | PRO | A | 303 | 51.218 | 38.261 | 32.931 | 1.00 | 51.75 | N | |
| ATOM | 2384 | CA | PRO | A | 303 | 50.107 | 37.797 | 33.785 | 1.00 | 51.76 | C | |
| ATOM | 2385 | C | PRO | A | 303 | 50.075 | 38.060 | 35.309 | 1.00 | 52.16 | C | |
| ATOM | 2386 | O | PRO | A | 303 | 49.830 | 39.178 | 35.766 | 1.00 | 52.37 | O | |
| ATOM | 2387 | CB | PRO | A | 303 | 50.037 | 36.300 | 33.458 | 1.00 | 52.46 | C | |
| ATOM | 2388 | CG | PRO | A | 303 | 50.358 | 36.273 | 32.022 | 1.00 | 53.97 | C | |
| ATOM | 2389 | CD | PRO | A | 303 | 51.553 | 37.219 | 31.934 | 1.00 | 53.46 | C | |
| ATOM | 2390 | N | GLU | A | 304 | 50.311 | 37.007 | 36.079 | 1.00 | 51.38 | N | |
| ATOM | 2391 | CA | GLU | A | 304 | 50.268 | 37.064 | 37.524 | 1.00 | 49.34 | C | |
| ATOM | 2392 | C | GLU | A | 304 | 51.408 | 37.730 | 38.323 | 1.00 | 46.24 | C | |
| ATOM | 2393 | O | GLU | A | 304 | 51.283 | 37.859 | 39.538 | 1.00 | 45.66 | O | |
| ATOM | 2394 | CB | GLU | A | 304 | 50.068 | 35.629 | 38.030 | 1.00 | 52.96 | C | |
| ATOM | 2395 | CG | GLU | A | 304 | 48.646 | 35.091 | 37.860 | 1.00 | 58.63 | C | |
| ATOM | 2396 | CD | GLU | A | 304 | 47.712 | 35.592 | 38.964 | 1.00 | 62.56 | C | |
| ATOM | 2397 | OE1 | GLU | A | 304 | 46.559 | 35.102 | 39.069 | 1.00 | 65.41 | O | |
| ATOM | 2398 | OE2 | GLU | A | 304 | 48.140 | 36.479 | 39.737 | 1.00 | 65.37 | O | |
| ATOM | 2399 | N | VAL | A | 305 | 52.480 | 38.187 | 37.673 | 1.00 | 41.53 | N | |
| ATOM | 2400 | CA | VAL | A | 305 | 53.623 | 38.747 | 38.419 | 1.00 | 40.51 | C | |
| ATOM | 2401 | C | VAL | A | 305 | 53.397 | 39.804 | 39.500 | 1.00 | 40.68 | C | |
| ATOM | 2402 | O | VAL | A | 305 | 53.794 | 39.578 | 40.654 | 1.00 | 38.26 | O | |
| ATOM | 2403 | CB | VAL | A | 305 | 54.736 | 39.295 | 37.486 | 1.00 | 38.34 | C | |
| ATOM | 2404 | CG1 | VAL | A | 305 | 55.968 | 39.636 | 38.307 | 1.00 | 37.60 | C | |
| ATOM | 2405 | CG2 | VAL | A | 305 | 55.100 | 38.280 | 36.455 | 1.00 | 35.65 | C | |
| ATOM | 2406 | N | ALA | A | 306 | 52.800 | 40.952 | 39.139 | 1.00 | 41.04 | N | |
| ATOM | 2407 | CA | ALA | A | 306 | 52.542 | 42.038 | 40.095 | 1.00 | 39.81 | C | |
| ATOM | 2408 | C | ALA | A | 306 | 51.583 | 41.577 | 41.191 | 1.00 | 40.47 | C | |
| ATOM | 2409 | O | ALA | A | 306 | 51.701 | 41.972 | 42.346 | 1.00 | 41.63 | O | |
| ATOM | 2410 | CB | ALA | A | 306 | 51.962 | 43.244 | 39.368 | 1.00 | 40.00 | C | |
| ATOM | 2411 | N | LYS | A | 307 | 50.651 | 40.717 | 40.812 | 1.00 | 40.77 | N | |
| ATOM | 2412 | CA | LYS | A | 307 | 49.652 | 40.167 | 41.717 | 1.00 | 43.28 | C | |
| ATOM | 2413 | C | LYS | A | 307 | 50.292 | 39.184 | 42.684 | 1.00 | 41.59 | C | |
| ATOM | 2414 | O | LYS | A | 307 | 50.220 | 39.354 | 43.907 | 1.00 | 42.23 | O | |
| ATOM | 2415 | CB | LYS | A | 307 | 48.580 | 39.415 | 40.913 | 1.00 | 45.70 | C | |
| ATOM | 2416 | CG | LYS | A | 307 | 48.661 | 39.659 | 39.400 | 1.00 | 49.68 | C | |
| ATOM | 2417 | CD | LYS | A | 307 | 47.624 | 40.686 | 38.932 | 1.00 | 51.15 | C | |
| ATOM | 2418 | CE | LYS | A | 307 | 46.358 | 39.997 | 38.402 | 1.00 | 51.48 | C | |
| ATOM | 2419 | NZ | LYS | A | 307 | 45.320 | 41.010 | 38.052 | 1.00 | 52.95 | N | |
| ATOM | 2420 | N | LYS | A | 308 | 50.916 | 38.151 | 42.120 | 1.00 | 39.65 | N | |
| ATOM | 2421 | CA | LYS | A | 308 | 51.538 | 37.120 | 42.928 | 1.00 | 38.61 | C | |
| ATOM | 2422 | C | LYS | A | 308 | 52.708 | 37.628 | 43.785 | 1.00 | 36.78 | C | |
| ATOM | 2423 | O | LYS | A | 308 | 52.874 | 37.183 | 44.912 | 1.00 | 34.50 | O | |
| ATOM | 2424 | CB | LYS | A | 308 | 51.961 | 35.961 | 42.025 | 1.00 | 41.97 | C | |
| ATOM | 2425 | CG | LYS | A | 308 | 52.064 | 34.632 | 42.754 | 1.00 | 48.52 | C | |
| ATOM | 2426 | CD | LYS | A | 308 | 52.268 | 33.459 | 41.809 | 1.00 | 50.33 | C | |
| ATOM | 2427 | CE | LYS | A | 308 | 52.864 | 32.276 | 42.573 | 1.00 | 53.68 | C | |
| ATOM | 2428 | NZ | LYS | A | 308 | 52.058 | 31.912 | 43.780 | 1.00 | 55.31 | N | |
| ATOM | 2429 | N | PHE | A | 309 | 53.501 | 38.566 | 43.265 | 1.00 | 33.40 | N | |
| ATOM | 2430 | CA | PHE | A | 309 | 54.640 | 39.120 | 44.009 | 1.00 | 33.19 | C | |
| ATOM | 2431 | C | PHE | A | 309 | 54.640 | 40.652 | 44.006 | 1.00 | 34.89 | C | |
| ATOM | 2432 | O | PHE | A | 309 | 55.415 | 41.296 | 43.308 | 1.00 | 32.46 | O | |
| ATOM | 2433 | CB | PHE | A | 309 | 55.936 | 38.602 | 43.394 | 1.00 | 30.00 | C | |
| ATOM | 2434 | CG | PHE | A | 309 | 56.036 | 37.122 | 43.387 | 1.00 | 29.40 | C | |
| ATOM | 2435 | CD1 | PHE | A | 309 | 56.524 | 36.441 | 44.492 | 1.00 | 30.17 | C | |
| ATOM | 2436 | CD2 | PHE | A | 309 | 55.631 | 36.387 | 42.258 | 1.00 | 28.30 | C | |
| ATOM | 2437 | CE1 | PHE | A | 309 | 56.615 | 35.051 | 44.478 | 1.00 | 31.47 | C | |
| ATOM | 2438 | CE2 | PHE | A | 309 | 55.718 | 35.005 | 42.235 | 1.00 | 28.17 | C | |
| ATOM | 2439 | CZ | PHE | A | 309 | 56.220 | 34.321 | 43.361 | 1.00 | 27.75 | C | |
| ATOM | 2440 | N | PRO | A | 310 | 53.787 | 41.254 | 44.841 | 1.00 | 35.46 | N | |
| ATOM | 2441 | CA | PRO | A | 310 | 53.664 | 42.701 | 44.939 | 1.00 | 34.26 | C | |
| ATOM | 2442 | C | PRO | A | 310 | 54.858 | 43.493 | 45.436 | 1.00 | 33.31 | C | |
| ATOM | 2443 | O | PRO | A | 310 | 55.437 | 43.169 | 46.499 | 1.00 | 31.12 | O | |
| ATOM | 2444 | CB | PRO | A | 310 | 52.482 | 42.889 | 45.914 | 1.00 | 36.60 | C | |
| ATOM | 2445 | CG | PRO | A | 310 | 51.755 | 41.599 | 45.896 | 1.00 | 40.32 | C | |
| ATOM | 2446 | CD | PRO | A | 310 | 52.849 | 40.577 | 45.756 | 1.00 | 36.81 | C | |
| ATOM | 2447 | N | ASN | A | 311 | 55.180 | 44.559 | 44.692 | 1.00 | 32.36 | N | |
| ATOM | 2448 | CA | ASN | A | 311 | 56.232 | 45.519 | 45.070 | 1.00 | 35.60 | C | |
| ATOM | 2449 | C | ASN | A | 311 | 57.549 | 44.879 | 45.457 | 1.00 | 35.30 | C | |
| ATOM | 2450 | O | ASN | A | 311 | 58.126 | 45.169 | 46.534 | 1.00 | 35.48 | O | |
| ATOM | 2451 | CB | ASN | A | 311 | 55.740 | 46.400 | 46.240 | 1.00 | 37.73 | C | |
| ATOM | 2452 | CG | ASN | A | 311 | 54.414 | 47.120 | 45.929 | 1.00 | 42.62 | C | |
| ATOM | 2453 | OD1 | ASN | A | 311 | 54.336 | 47.962 | 45.025 | 1.00 | 42.81 | O | |
| ATOM | 2454 | ND2 | ASN | A | 311 | 53.370 | 46.778 | 46.678 | 1.00 | 41.05 | N | |
| ATOM | 2455 | N | MET | A | 312 | 57.995 | 43.973 | 44.595 | 1.00 | 34.53 | N | |
| ATOM | 2456 | CA | MET | A | 312 | 59.249 | 43.263 | 44.795 | 1.00 | 32.63 | C | |
| ATOM | 2457 | C | MET | A | 312 | 60.224 | 43.561 | 43.660 | 1.00 | 30.68 | C | |
| ATOM | 2458 | O | MET | A | 312 | 59.823 | 43.910 | 42.550 | 1.00 | 30.81 | O | |
| ATOM | 2459 | CB | MET | A | 312 | 58.984 | 41.762 | 44.843 | 1.00 | 31.25 | C | |
| ATOM | 2460 | CG | MET | A | 312 | 58.082 | 41.357 | 45.978 | 1.00 | 33.53 | C | |
| ATOM | 2461 | SD | MET | A | 312 | 58.119 | 39.621 | 46.270 | 1.00 | 33.54 | S | |
| ATOM | 2462 | CE | MET | A | 312 | 56.901 | 39.539 | 47.577 | 1.00 | 33.09 | C | |
| ATOM | 2463 | N | LYS | A | 313 | 61.507 | 43.436 | 43.958 | 1.00 | 29.20 | N | |
| ATOM | 2464 | CA | LYS | A | 313 | 62.516 | 43.652 | 42.942 | 1.00 | 30.48 | C | |
| ATOM | 2465 | C | LYS | A | 313 | 62.647 | 42.329 | 42.156 | 1.00 | 29.98 | C | |
| ATOM | 2466 | O | LYS | A | 313 | 62.196 | 41.268 | 42.609 | 1.00 | 28.89 | O | |
| ATOM | 2467 | CB | LYS | A | 313 | 63.830 | 44.037 | 43.594 | 1.00 | 33.24 | C | |
| ATOM | 2468 | CG | LYS | A | 313 | 63.703 | 45.154 | 44.628 | 1.00 | 35.74 | C | |
| ATOM | 2469 | CD | LYS | A | 313 | 65.037 | 45.852 | 44.785 | 1.00 | 39.90 | C | |
| ATOM | 2470 | CE | LYS | A | 313 | 65.321 | 46.696 | 43.538 | 1.00 | 42.68 | C | |
| ATOM | 2471 | NZ | LYS | A | 313 | 66.753 | 46.669 | 43.090 | 1.00 | 46.41 | N | |
| ATOM | 2472 | N | ILE | A | 314 | 63.215 | 42.398 | 40.963 | 1.00 | 28.60 | N | |
| ATOM | 2473 | CA | ILE | A | 314 | 63.377 | 41.176 | 40.173 | 1.00 | 26.52 | C | |
| ATOM | 2474 | C | ILE | A | 314 | 64.835 | 41.022 | 39.796 | 1.00 | 23.53 | C | |
| ATOM | 2475 | O | ILE | A | 314 | 65.547 | 42.003 | 39.610 | 1.00 | 23.19 | O | |
| ATOM | 2476 | CB | ILE | A | 314 | 62.521 | 41.219 | 38.849 | 1.00 | 24.04 | C | |
| ATOM | 2477 | CG1 | ILE | A | 314 | 61.078 | 40.895 | 39.143 | 1.00 | 26.40 | C | |
| ATOM | 2478 | CG2 | ILE | A | 314 | 62.998 | 40.176 | 37.805 | 1.00 | 24.56 | C | |
| ATOM | 2479 | CD1 | ILE | A | 314 | 60.182 | 40.980 | 37.875 | 1.00 | 26.30 | C | |
| ATOM | 2480 | N | GLN | A | 315 | 65.295 | 39.773 | 39.704 | 1.00 | 23.51 | N | |
| ATOM | 2481 | CA | GLN | A | 315 | 66.665 | 39.532 | 39.272 | 1.00 | 19.49 | C | |
| ATOM | 2482 | C | GLN | A | 315 | 66.580 | 38.562 | 38.074 | 1.00 | 19.49 | C | |
| ATOM | 2483 | O | GLN | A | 315 | 65.699 | 37.710 | 38.035 | 1.00 | 18.23 | O | |
| ATOM | 2484 | CB | GLN | A | 315 | 67.495 | 38.835 | 40.365 | 1.00 | 24.40 | C | |
| ATOM | 2485 | CG | GLN | A | 315 | 67.744 | 39.589 | 41.653 | 1.00 | 26.52 | C | |
| ATOM | 2486 | CD | GLN | A | 315 | 68.850 | 38.933 | 42.444 | 1.00 | 27.12 | C | |
| ATOM | 2487 | OE1 | GLN | A | 315 | 69.378 | 37.875 | 42.054 | 1.00 | 25.45 | O | |
| ATOM | 2488 | NE2 | GLN | A | 315 | 69.217 | 39.556 | 43.560 | 1.00 | 26.68 | N | |
| ATOM | 2489 | N | ILE | A | 316 | 67.478 | 38.736 | 37.097 | 1.00 | 22.89 | N | |
| ATOM | 2490 | CA | ILE | A | 316 | 67.509 | 37.894 | 35.897 | 1.00 | 19.87 | C | |
| ATOM | 2491 | C | ILE | A | 316 | 68.880 | 37.254 | 35.892 | 1.00 | 23.17 | C | |
| ATOM | 2492 | O | ILE | A | 316 | 69.886 | 37.961 | 35.835 | 1.00 | 23.46 | O | |
| ATOM | 2493 | CB | ILE | A | 316 | 67.326 | 38.742 | 34.601 | 1.00 | 24.31 | C | |
| ATOM | 2494 | CG1 | ILE | A | 316 | 65.891 | 39.292 | 34.530 | 1.00 | 24.19 | C | |
| ATOM | 2495 | CG2 | ILE | A | 316 | 67.594 | 37.892 | 33.372 | 1.00 | 23.47 | C | |
| ATOM | 2496 | CD1 | ILE | A | 316 | 65.567 | 39.918 | 33.215 | 1.00 | 29.77 | C | |
| ATOM | 2497 | N | HIS | A | 317 | 68.918 | 35.921 | 35.975 | 1.00 | 19.09 | N | |
| ATOM | 2498 | CA | HIS | A | 317 | 70.172 | 35.177 | 35.955 | 1.00 | 16.10 | C | |
| ATOM | 2499 | C | HIS | A | 317 | 70.358 | 34.556 | 34.545 | 1.00 | 18.62 | C | |
| ATOM | 2500 | O | HIS | A | 317 | 69.503 | 33.839 | 34.049 | 1.00 | 17.53 | O | |
| ATOM | 2501 | CB | HIS | A | 317 | 70.120 | 34.054 | 37.024 | 1.00 | 14.39 | C | |
| ATOM | 2502 | CG | HIS | A | 317 | 70.067 | 34.539 | 38.451 | 1.00 | 21.51 | C | |
| ATOM | 2503 | ND1 | HIS | A | 317 | 70.304 | 33.696 | 39.522 | 1.00 | 20.77 | N | |
| ATOM | 2504 | CD2 | HIS | A | 317 | 69.791 | 35.757 | 38.989 | 1.00 | 22.21 | C | |
| ATOM | 2505 | CE1 | HIS | A | 317 | 70.183 | 34.372 | 40.653 | 1.00 | 18.38 | C | |
| ATOM | 2506 | NE2 | HIS | A | 317 | 69.872 | 35.625 | 40.361 | 1.00 | 23.87 | N | |
| ATOM | 2507 | N | VAL | A | 318 | 71.513 | 34.814 | 33.948 | 1.00 | 18.27 | N | |
| ATOM | 2508 | CA | VAL | A | 318 | 71.864 | 34.363 | 32.620 | 1.00 | 17.81 | C | |
| ATOM | 2509 | C | VAL | A | 318 | 72.975 | 33.343 | 32.792 | 1.00 | 19.49 | C | |
| ATOM | 2510 | O | VAL | A | 318 | 73.951 | 33.603 | 33.529 | 1.00 | 19.00 | O | |
| ATOM | 2511 | CB | VAL | A | 318 | 72.414 | 35.556 | 31.788 | 1.00 | 20.65 | C | |
| ATOM | 2512 | CG1 | VAL | A | 318 | 72.833 | 35.063 | 30.396 | 1.00 | 26.11 | C | |
| ATOM | 2513 | CG2 | VAL | A | 318 | 71.338 | 36.635 | 31.660 | 1.00 | 23.64 | C | |
| ATOM | 2514 | N | SER | A | 319 | 72.843 | 32.207 | 32.121 | 1.00 | 18.81 | N | |
| ATOM | 2515 | CA | SER | A | 319 | 73.887 | 31.194 | 32.234 | 1.00 | 22.45 | C | |
| ATOM | 2516 | C | SER | A | 319 | 73.927 | 30.236 | 31.052 | 1.00 | 22.89 | C | |
| ATOM | 2517 | O | SER | A | 319 | 72.997 | 30.146 | 30.255 | 1.00 | 22.67 | O | |
| ATOM | 2518 | CB | SER | A | 319 | 73.715 | 30.407 | 33.567 | 1.00 | 25.56 | C | |
| ATOM | 2519 | OG | SER | A | 319 | 72.466 | 29.700 | 33.653 | 1.00 | 28.71 | O | |
| ATOM | 2520 | N | ALA | A | 320 | 75.055 | 29.533 | 30.929 | 1.00 | 26.30 | N | |
| ATOM | 2521 | CA | ALA | A | 320 | 75.216 | 28.546 | 29.869 | 1.00 | 27.76 | C | |
| ATOM | 2522 | C | ALA | A | 320 | 75.246 | 27.155 | 30.538 | 1.00 | 28.34 | C | |
| ATOM | 2523 | O | ALA | A | 320 | 76.151 | 26.852 | 31.314 | 1.00 | 30.21 | O | |
| ATOM | 2524 | CB | ALA | A | 320 | 76.547 | 28.827 | 29.097 | 1.00 | 26.62 | C | |
| ATOM | 2525 | N | SER | A | 321 | 74.240 | 26.327 | 30.286 | 1.00 | 26.00 | N | |
| ATOM | 2526 | CA | SER | A | 321 | 74.269 | 24.996 | 30.872 | 1.00 | 30.50 | C | |
| ATOM | 2527 | C | SER | A | 321 | 74.933 | 23.963 | 29.948 | 1.00 | 32.26 | C | |
| ATOM | 2528 | O | SER | A | 321 | 75.077 | 22.779 | 30.315 | 1.00 | 34.46 | O | |
| ATOM | 2529 | CB | SER | A | 321 | 72.862 | 24.550 | 31.307 | 1.00 | 30.34 | C | |
| ATOM | 2530 | OG | SER | A | 321 | 71.953 | 24.477 | 30.229 | 1.00 | 30.32 | O | |
| ATOM | 2531 | N | THR | A | 322 | 75.316 | 24.405 | 28.749 | 1.00 | 33.10 | N | |
| ATOM | 2532 | CA | THR | A | 322 | 76.043 | 23.587 | 27.789 | 1.00 | 32.40 | C | |
| ATOM | 2533 | C | THR | A | 322 | 77.139 | 24.461 | 27.166 | 1.00 | 29.30 | C | |
| ATOM | 2534 | O | THR | A | 322 | 76.952 | 25.641 | 26.909 | 1.00 | 25.36 | O | |
| ATOM | 2535 | CB | THR | A | 322 | 75.153 | 22.999 | 26.657 | 1.00 | 37.96 | C | |
| ATOM | 2536 | OG1 | THR | A | 322 | 74.683 | 24.052 | 25.807 | 1.00 | 39.60 | O | |
| ATOM | 2537 | CG2 | THR | A | 322 | 73.976 | 22.214 | 27.249 | 1.00 | 39.13 | C | |
| ATOM | 2538 | N | PRO | A | 323 | 78.308 | 23.877 | 26.909 | 1.00 | 29.98 | N | |
| ATOM | 2539 | CA | PRO | A | 323 | 79.428 | 24.623 | 26.324 | 1.00 | 28.92 | C | |
| ATOM | 2540 | C | PRO | A | 323 | 79.097 | 25.136 | 24.937 | 1.00 | 28.68 | C | |
| ATOM | 2541 | O | PRO | A | 323 | 78.577 | 24.402 | 24.105 | 1.00 | 27.85 | O | |
| ATOM | 2542 | CB | PRO | A | 323 | 80.554 | 23.595 | 26.337 | 1.00 | 30.45 | C | |
| ATOM | 2543 | CG | PRO | A | 323 | 79.806 | 22.339 | 26.000 | 1.00 | 36.44 | C | |
| ATOM | 2544 | CD | PRO | A | 323 | 78.579 | 22.435 | 26.919 | 1.00 | 32.60 | C | |
| ATOM | 2545 | N | PRO | A | 324 | 79.365 | 26.427 | 24.681 | 1.00 | 28.46 | N | |
| ATOM | 2546 | CA | PRO | A | 324 | 79.064 | 26.998 | 23.368 | 1.00 | 29.04 | C | |
| ATOM | 2547 | C | PRO | A | 324 | 79.972 | 26.475 | 22.249 | 1.00 | 30.07 | C | |
| ATOM | 2548 | O | PRO | A | 324 | 81.197 | 26.412 | 22.409 | 1.00 | 30.23 | O | |
| ATOM | 2549 | CB | PRO | A | 324 | 79.183 | 28.503 | 23.609 | 1.00 | 28.06 | C | |
| ATOM | 2550 | CG | PRO | A | 324 | 80.289 | 28.585 | 24.660 | 1.00 | 29.33 | C | |
| ATOM | 2551 | CD | PRO | A | 324 | 80.001 | 27.411 | 25.584 | 1.00 | 28.96 | C | |
| ATOM | 2552 | N | AHIS | A | 325 | 79.374 | 26.098 | 21.121 | 0.50 | 27.02 | N | |
| ATOM | 2553 | N | BHIS | A | 325 | 79.336 | 26.102 | 21.137 | 0.50 | 28.68 | N | |
| ATOM | 2554 | CA | AHIS | A | 325 | 80.155 | 25.578 | 20.000 | 0.50 | 26.87 | C | |
| ATOM | 2555 | CA | BHIS | A | 325 | 80.020 | 25.567 | 19.962 | 0.50 | 30.05 | C | |
| ATOM | 2556 | C | AHIS | A | 325 | 80.552 | 26.662 | 18.994 | 0.50 | 24.15 | C | |
| ATOM | 2557 | C | BHIS | A | 325 | 80.525 | 26.665 | 19.019 | 0.50 | 27.92 | C | |
| ATOM | 2558 | O | AHIS | A | 325 | 79.858 | 27.669 | 18.837 | 0.50 | 24.29 | O | |
| ATOM | 2559 | O | BHIS | A | 325 | 79.975 | 27.766 | 18.967 | 0.50 | 28.29 | O | |
| ATOM | 2560 | CB | AHIS | A | 325 | 79.366 | 24.509 | 19.223 | 0.50 | 30.59 | C | |
| ATOM | 2561 | CB | BHIS | A | 325 | 79.066 | 24.676 | 19.140 | 0.50 | 33.50 | C | |
| ATOM | 2562 | CG | AHIS | A | 325 | 78.627 | 23.526 | 20.079 | 0.50 | 32.95 | C | |
| ATOM | 2563 | CG | BHIS | A | 325 | 78.432 | 23.557 | 19.911 | 0.50 | 37.48 | C | |
| ATOM | 2564 | ND1 | AHIS | A | 325 | 79.131 | 23.024 | 21.259 | 0.50 | 32.44 | N | |
| ATOM | 2565 | ND1 | BHIS | A | 325 | 77.512 | 23.767 | 20.917 | 0.50 | 38.53 | N | |
| ATOM | 2566 | CD2 | AHIS | A | 325 | 77.437 | 22.906 | 19.887 | 0.50 | 34.27 | C | |
| ATOM | 2567 | CD2 | BHIS | A | 325 | 78.550 | 22.213 | 19.788 | 0.50 | 39.34 | C | |
| ATOM | 2568 | CE1 | AHIS | A | 325 | 78.288 | 22.138 | 21.757 | 0.50 | 34.55 | C | |
| ATOM | 2569 | CE1 | BHIS | A | 325 | 77.091 | 22.603 | 21.379 | 0.50 | 40.83 | C | |
| ATOM | 2570 | NE2 | AHIS | A | 325 | 77.250 | 22.048 | 20.942 | 0.50 | 36.30 | N | |
| ATOM | 2571 | NE2 | BHIS | A | 325 | 77.706 | 21.643 | 20.710 | 0.50 | 41.15 | N | |
| ATOM | 2572 | N | ALEU | A | 326 | 81.650 | 26.415 | 18.282 | 0.50 | 20.47 | N | |
| ATOM | 2573 | N | BLEU | A | 326 | 81.549 | 26.331 | 18.241 | 0.50 | 25.62 | N | |
| ATOM | 2574 | CA | ALEU | A | 326 | 82.135 | 27.298 | 17.236 | 0.50 | 21.63 | C | |
| ATOM | 2575 | CA | BLEU | A | 326 | 82.103 | 27.226 | 17.244 | 0.50 | 25.44 | C | |
| ATOM | 2576 | C | ALEU | A | 326 | 82.497 | 26.461 | 16.001 | 0.50 | 21.24 | C | |
| ATOM | 2577 | C | BLEU | A | 326 | 82.469 | 26.421 | 15.997 | 0.50 | 23.88 | C | |
| ATOM | 2578 | O | ALEU | A | 326 | 83.091 | 25.386 | 16.130 | 0.50 | 22.62 | O | |
| ATOM | 2579 | O | BLEU | A | 326 | 83.050 | 25.336 | 16.108 | 0.50 | 24.48 | O | |
| ATOM | 2580 | CB | ALEU | A | 326 | 83.371 | 28.072 | 17.722 | 0.50 | 22.23 | C | |
| ATOM | 2581 | CB | BLEU | A | 326 | 83.344 | 27.938 | 17.789 | 0.50 | 28.35 | C | |
| ATOM | 2582 | CG | ALEU | A | 326 | 84.043 | 29.125 | 16.836 | 0.50 | 22.02 | C | |
| ATOM | 2583 | CG | BLEU | A | 326 | 83.057 | 29.274 | 18.463 | 0.50 | 30.00 | C | |
| ATOM | 2584 | CD1 | ALEU | A | 326 | 84.836 | 30.039 | 17.757 | 0.50 | 21.82 | C | |
| ATOM | 2585 | CD1 | BLEU | A | 326 | 84.089 | 29.554 | 19.537 | 0.50 | 33.17 | C | |
| ATOM | 2586 | CD2 | ALEU | A | 326 | 84.943 | 28.512 | 15.774 | 0.50 | 24.66 | C | |
| ATOM | 2587 | CD2 | BLEU | A | 326 | 83.046 | 30.373 | 17.398 | 0.50 | 30.86 | C | |
| ATOM | 2588 | N | SER | A | 327 | 82.083 | 26.910 | 14.819 | 1.00 | 21.24 | N | |
| ATOM | 2589 | CA | SER | A | 327 | 82.457 | 26.209 | 13.584 | 1.00 | 20.71 | C | |
| ATOM | 2590 | C | SER | A | 327 | 83.269 | 27.160 | 12.689 | 1.00 | 20.90 | C | |
| ATOM | 2591 | O | SER | A | 327 | 83.187 | 28.428 | 12.783 | 1.00 | 20.02 | O | |
| ATOM | 2592 | CB | SER | A | 327 | 81.255 | 25.604 | 12.814 | 1.00 | 23.19 | C | |
| ATOM | 2593 | OG | SER | A | 327 | 80.484 | 26.607 | 12.255 | 1.00 | 29.56 | O | |
| ATOM | 2594 | N | VAL | A | 328 | 84.156 | 26.577 | 11.886 | 1.00 | 20.65 | N | |
| ATOM | 2595 | CA | VAL | A | 328 | 84.971 | 27.380 | 10.959 | 1.00 | 22.43 | C | |
| ATOM | 2596 | C | VAL | A | 328 | 84.650 | 26.921 | 9.535 | 1.00 | 25.95 | C | |
| ATOM | 2597 | O | VAL | A | 328 | 84.693 | 25.734 | 9.251 | 1.00 | 24.62 | O | |
| ATOM | 2598 | CB | VAL | A | 328 | 86.492 | 27.160 | 11.150 | 1.00 | 23.21 | C | |
| ATOM | 2599 | CG1 | VAL | A | 328 | 87.286 | 28.200 | 10.364 | 1.00 | 24.86 | C | |
| ATOM | 2600 | CG2 | VAL | A | 328 | 86.824 | 27.216 | 12.582 | 1.00 | 24.31 | C | |
| ATOM | 2601 | N | GLN | A | 329 | 84.320 | 27.881 | 8.677 | 1.00 | 27.90 | N | |
| ATOM | 2602 | CA | GLN | A | 329 | 84.008 | 27.640 | 7.262 | 1.00 | 31.26 | C | |
| ATOM | 2603 | C | GLN | A | 329 | 84.818 | 28.672 | 6.496 | 1.00 | 29.53 | C | |
| ATOM | 2604 | O | GLN | A | 329 | 85.318 | 29.624 | 7.079 | 1.00 | 29.67 | O | |
| ATOM | 2605 | CB | GLN | A | 329 | 82.532 | 27.952 | 6.961 | 1.00 | 32.70 | C | |
| ATOM | 2606 | CG | GLN | A | 329 | 81.500 | 27.136 | 7.686 | 1.00 | 40.54 | C | |
| ATOM | 2607 | CD | GLN | A | 329 | 81.306 | 27.511 | 9.158 | 1.00 | 41.37 | C | |
| ATOM | 2608 | OE1 | GLN | A | 329 | 81.204 | 26.624 | 9.992 | 1.00 | 42.86 | O | |
| ATOM | 2609 | NE2 | GLN | A | 329 | 81.233 | 28.827 | 9.477 | 1.00 | 41.42 | N | |
| ATOM | 2610 | N | PRO | A | 330 | 84.904 | 28.535 | 5.152 | 1.00 | 33.50 | N | |
| ATOM | 2611 | CA | PRO | A | 330 | 85.660 | 29.506 | 4.354 | 1.00 | 33.92 | C | |
| ATOM | 2612 | C | PRO | A | 330 | 85.120 | 30.897 | 4.575 | 1.00 | 33.13 | C | |
| ATOM | 2613 | O | PRO | A | 330 | 85.830 | 31.864 | 4.384 | 1.00 | 35.50 | O | |
| ATOM | 2614 | CB | PRO | A | 330 | 85.453 | 29.007 | 2.928 | 1.00 | 36.48 | C | |
| ATOM | 2615 | CG | PRO | A | 330 | 85.490 | 27.507 | 3.135 | 1.00 | 35.94 | C | |
| ATOM | 2616 | CD | PRO | A | 330 | 84.550 | 27.362 | 4.330 | 1.00 | 32.54 | C | |
| ATOM | 2617 | N | THR | A | 331 | 83.873 | 30.975 | 5.039 | 1.00 | 36.17 | N | |
| ATOM | 2618 | CA | THR | A | 331 | 83.200 | 32.248 | 5.325 | 1.00 | 40.14 | C | |
| ATOM | 2619 | C | THR | A | 331 | 83.712 | 32.945 | 6.607 | 1.00 | 41.02 | C | |
| ATOM | 2620 | O | THR | A | 331 | 83.540 | 34.164 | 6.794 | 1.00 | 43.07 | O | |
| ATOM | 2621 | CB | THR | A | 331 | 81.659 | 32.036 | 5.459 | 1.00 | 41.98 | C | |
| ATOM | 2622 | OG1 | THR | A | 331 | 80.974 | 33.258 | 5.153 | 1.00 | 46.94 | O | |
| ATOM | 2623 | CG2 | THR | A | 331 | 81.271 | 31.640 | 6.902 | 1.00 | 45.95 | C | |
| ATOM | 2624 | N | GLY | A | 332 | 84.344 | 32.178 | 7.491 | 1.00 | 36.57 | N | |
| ATOM | 2625 | CA | GLY | A | 332 | 84.829 | 32.755 | 8.734 | 1.00 | 36.42 | C | |
| ATOM | 2626 | C | GLY | A | 332 | 84.416 | 31.872 | 9.907 | 1.00 | 35.91 | C | |
| ATOM | 2627 | O | GLY | A | 332 | 83.954 | 30.736 | 9.701 | 1.00 | 36.09 | O | |
| ATOM | 2628 | N | LEU | A | 333 | 84.591 | 32.371 | 11.127 | 1.00 | 33.41 | N | |
| ATOM | 2629 | CA | LEU | A | 333 | 84.225 | 31.613 | 12.314 | 1.00 | 31.55 | C | |
| ATOM | 2630 | C | LEU | A | 333 | 82.835 | 31.997 | 12.762 | 1.00 | 31.35 | C | |
| ATOM | 2631 | O | LEU | A | 333 | 82.464 | 33.178 | 12.773 | 1.00 | 28.77 | O | |
| ATOM | 2632 | CB | LEU | A | 333 | 85.187 | 31.878 | 13.471 | 1.00 | 33.13 | C | |
| ATOM | 2633 | CG | LEU | A | 333 | 86.674 | 31.568 | 13.386 | 1.00 | 38.69 | C | |
| ATOM | 2634 | CD1 | LEU | A | 333 | 87.311 | 31.764 | 14.771 | 1.00 | 40.78 | C | |
| ATOM | 2635 | CD2 | LEU | A | 333 | 86.870 | 30.128 | 12.957 | 1.00 | 42.94 | C | |
| ATOM | 2636 | N | THR | A | 334 | 82.059 | 30.988 | 13.130 | 1.00 | 27.54 | N | |
| ATOM | 2637 | CA | THR | A | 334 | 80.701 | 31.188 | 13.591 | 1.00 | 27.09 | C | |
| ATOM | 2638 | C | THR | A | 334 | 80.538 | 30.590 | 14.970 | 1.00 | 27.41 | C | |
| ATOM | 2639 | O | THR | A | 334 | 80.984 | 29.478 | 15.228 | 1.00 | 25.09 | O | |
| ATOM | 2640 | CB | THR | A | 334 | 79.762 | 30.535 | 12.642 | 1.00 | 29.52 | C | |
| ATOM | 2641 | OG1 | THR | A | 334 | 79.990 | 31.094 | 11.346 | 1.00 | 34.40 | O | |
| ATOM | 2642 | CG2 | THR | A | 334 | 78.355 | 30.775 | 13.051 | 1.00 | 30.58 | C | |
| ATOM | 2643 | N | PHE | A | 335 | 79.867 | 31.335 | 15.845 | 1.00 | 23.79 | N | |
| ATOM | 2644 | CA | PHE | A | 335 | 79.681 | 30.952 | 17.234 | 1.00 | 23.65 | C | |
| ATOM | 2645 | C | PHE | A | 335 | 78.189 | 30.709 | 17.438 | 1.00 | 22.59 | C | |
| ATOM | 2646 | O | PHE | A | 335 | 77.375 | 31.479 | 16.938 | 1.00 | 22.87 | O | |
| ATOM | 2647 | CB | PHE | A | 335 | 80.169 | 32.159 | 18.084 | 1.00 | 23.69 | C | |
| ATOM | 2648 | CG | PHE | A | 335 | 80.193 | 31.915 | 19.531 | 1.00 | 28.04 | C | |
| ATOM | 2649 | CD1 | PHE | A | 335 | 81.066 | 30.971 | 20.065 | 1.00 | 34.71 | C | |
| ATOM | 2650 | CD2 | PHE | A | 335 | 79.437 | 32.697 | 20.387 | 1.00 | 29.22 | C | |
| ATOM | 2651 | CE1 | PHE | A | 335 | 81.197 | 30.815 | 21.450 | 1.00 | 35.43 | C | |
| ATOM | 2652 | CE2 | PHE | A | 335 | 79.550 | 32.555 | 21.770 | 1.00 | 33.78 | C | |
| ATOM | 2653 | CZ | PHE | A | 335 | 80.432 | 31.613 | 22.304 | 1.00 | 34.26 | C | |
| ATOM | 2654 | N | TYR | A | 336 | 77.810 | 29.652 | 18.152 | 1.00 | 22.23 | N | |
| ATOM | 2655 | CA | TYR | A | 336 | 76.387 | 29.341 | 18.347 | 1.00 | 24.31 | C | |
| ATOM | 2656 | C | TYR | A | 336 | 76.041 | 29.393 | 19.826 | 1.00 | 26.09 | C | |
| ATOM | 2657 | O | TYR | A | 336 | 75.847 | 28.362 | 20.443 | 1.00 | 27.05 | O | |
| ATOM | 2658 | CB | TYR | A | 336 | 76.086 | 27.937 | 17.819 | 1.00 | 26.23 | C | |
| ATOM | 2659 | CG | TYR | A | 336 | 76.487 | 27.762 | 16.396 | 1.00 | 26.77 | C | |
| ATOM | 2660 | CD1 | TYR | A | 336 | 75.656 | 28.154 | 15.350 | 1.00 | 27.06 | C | |
| ATOM | 2661 | CD2 | TYR | A | 336 | 77.728 | 27.240 | 16.089 | 1.00 | 27.25 | C | |
| ATOM | 2662 | CE1 | TYR | A | 336 | 76.082 | 28.021 | 14.024 | 1.00 | 31.18 | C | |
| ATOM | 2663 | CE2 | TYR | A | 336 | 78.153 | 27.108 | 14.796 | 1.00 | 27.24 | C | |
| ATOM | 2664 | CZ | TYR | A | 336 | 77.329 | 27.502 | 13.772 | 1.00 | 29.22 | C | |
| ATOM | 2665 | OH | TYR | A | 336 | 77.835 | 27.396 | 12.513 | 1.00 | 34.11 | O | |
| ATOM | 2666 | N | PRO | A | 337 | 75.903 | 30.599 | 20.387 | 1.00 | 25.03 | N | |
| ATOM | 2667 | CA | PRO | A | 337 | 75.580 | 30.704 | 21.816 | 1.00 | 27.13 | C | |
| ATOM | 2668 | C | PRO | A | 337 | 74.175 | 30.268 | 22.188 | 1.00 | 24.60 | C | |
| ATOM | 2669 | O | PRO | A | 337 | 73.223 | 30.522 | 21.438 | 1.00 | 25.96 | O | |
| ATOM | 2670 | CB | PRO | A | 337 | 75.863 | 32.182 | 22.134 | 1.00 | 25.32 | C | |
| ATOM | 2671 | CG | PRO | A | 337 | 75.585 | 32.894 | 20.838 | 1.00 | 25.23 | C | |
| ATOM | 2672 | CD | PRO | A | 337 | 76.156 | 31.924 | 19.786 | 1.00 | 25.35 | C | |
| ATOM | 2673 | N | ALA | A | 338 | 74.077 | 29.503 | 23.276 | 1.00 | 25.04 | N | |
| ATOM | 2674 | CA | ALA | A | 338 | 72.790 | 29.049 | 23.827 | 1.00 | 27.02 | C | |
| ATOM | 2675 | C | ALA | A | 338 | 72.838 | 29.381 | 25.337 | 1.00 | 25.47 | C | |
| ATOM | 2676 | O | ALA | A | 338 | 73.761 | 28.907 | 26.082 | 1.00 | 25.07 | O | |
| ATOM | 2677 | CB | ALA | A | 338 | 72.605 | 27.560 | 23.596 | 1.00 | 29.61 | C | |
| ATOM | 2678 | N | VAL | A | 339 | 71.880 | 30.202 | 25.788 | 1.00 | 23.49 | N | |
| ATOM | 2679 | CA | VAL | A | 339 | 71.850 | 30.638 | 27.185 | 1.00 | 24.36 | C | |
| ATOM | 2680 | C | VAL | A | 339 | 70.518 | 30.417 | 27.843 | 1.00 | 22.40 | C | |
| ATOM | 2681 | O | VAL | A | 339 | 69.490 | 30.375 | 27.174 | 1.00 | 20.04 | O | |
| ATOM | 2682 | CB | VAL | A | 339 | 72.291 | 32.148 | 27.329 | 1.00 | 31.73 | C | |
| ATOM | 2683 | CG1 | VAL | A | 339 | 71.490 | 33.029 | 26.479 | 1.00 | 30.59 | C | |
| ATOM | 2684 | CG2 | VAL | A | 339 | 72.130 | 32.612 | 28.722 | 1.00 | 35.06 | C | |
| ATOM | 2685 | N | ASP | A | 340 | 70.551 | 30.163 | 29.151 | 1.00 | 19.50 | N | |
| ATOM | 2686 | CA | ASP | A | 340 | 69.339 | 29.991 | 29.960 | 1.00 | 20.79 | C | |
| ATOM | 2687 | C | ASP | A | 340 | 69.120 | 31.349 | 30.611 | 1.00 | 18.51 | C | |
| ATOM | 2688 | O | ASP | A | 340 | 70.080 | 31.973 | 31.060 | 1.00 | 18.68 | O | |
| ATOM | 2689 | CB | ASP | A | 340 | 69.583 | 28.954 | 31.070 | 1.00 | 19.34 | C | |
| ATOM | 2690 | CG | ASP | A | 340 | 69.963 | 27.586 | 30.499 | 1.00 | 30.04 | C | |
| ATOM | 2691 | OD1 | ASP | A | 340 | 69.356 | 27.227 | 29.466 | 1.00 | 25.36 | O | |
| ATOM | 2692 | OD2 | ASP | A | 340 | 70.857 | 26.898 | 31.084 | 1.00 | 30.87 | O | |
| ATOM | 2693 | N | VAL | A | 341 | 67.878 | 31.826 | 30.645 | 1.00 | 19.16 | N | |
| ATOM | 2694 | CA | VAL | A | 341 | 67.617 | 33.116 | 31.288 | 1.00 | 17.44 | C | |
| ATOM | 2695 | C | VAL | A | 341 | 66.453 | 32.862 | 32.241 | 1.00 | 17.73 | C | |
| ATOM | 2696 | O | VAL | A | 341 | 65.372 | 32.467 | 31.808 | 1.00 | 17.27 | O | |
| ATOM | 2697 | CB | VAL | A | 341 | 67.161 | 34.195 | 30.262 | 1.00 | 19.03 | C | |
| ATOM | 2698 | CG1 | VAL | A | 341 | 66.926 | 35.521 | 30.976 | 1.00 | 20.90 | C | |
| ATOM | 2699 | CG2 | VAL | A | 341 | 68.239 | 34.395 | 29.167 | 1.00 | 20.47 | O | |
| ATOM | 2700 | N | GLN | A | 342 | 66.670 | 33.100 | 33.525 | 1.00 | 16.40 | N | |
| ATOM | 2701 | CA | GLN | A | 342 | 65.635 | 32.844 | 34.525 | 1.00 | 16.88 | C | |
| ATOM | 2702 | C | GLN | A | 342 | 65.378 | 34.102 | 35.358 | 1.00 | 18.10 | C | |
| ATOM | 2703 | O | GLN | A | 342 | 66.298 | 34.757 | 35.819 | 1.00 | 16.79 | O | |
| ATOM | 2704 | CB | GLN | A | 342 | 66.081 | 31.693 | 35.450 | 1.00 | 16.56 | C | |
| ATOM | 2705 | CG | GLN | A | 342 | 64.975 | 31.156 | 36.330 | 1.00 | 19.69 | C | |
| ATOM | 2706 | CD | GLN | A | 342 | 65.360 | 29.828 | 36.987 | 1.00 | 19.50 | C | |
| ATOM | 2707 | OE1 | GLN | A | 342 | 66.517 | 29.375 | 36.884 | 1.00 | 20.28 | O | |
| ATOM | 2708 | NE2 | GLN | A | 342 | 64.389 | 29.182 | 37.639 | 1.00 | 20.81 | N | |
| ATOM | 2709 | N | ALA | A | 343 | 64.106 | 34.422 | 35.558 | 1.00 | 17.71 | N | |
| ATOM | 2710 | CA | ALA | A | 343 | 63.722 | 35.612 | 36.278 | 1.00 | 18.52 | C | |
| ATOM | 2711 | C | ALA | A | 343 | 63.236 | 35.241 | 37.670 | 1.00 | 16.11 | C | |
| ATOM | 2712 | O | ALA | A | 343 | 62.529 | 34.247 | 37.819 | 1.00 | 20.52 | O | |
| ATOM | 2713 | CB | ALA | A | 343 | 62.586 | 36.285 | 35.510 | 1.00 | 22.15 | C | |
| ATOM | 2714 | N | PHE | A | 344 | 63.631 | 36.011 | 38.678 | 1.00 | 17.89 | N | |
| ATOM | 2715 | CA | PHE | A | 344 | 63.205 | 35.745 | 40.048 | 1.00 | 18.07 | C | |
| ATOM | 2716 | C | PHE | A | 344 | 62.639 | 36.987 | 40.751 | 1.00 | 18.31 | C | |
| ATOM | 2717 | O | PHE | A | 344 | 63.092 | 38.095 | 40.494 | 1.00 | 22.64 | O | |
| ATOM | 2718 | CB | PHE | A | 344 | 64.408 | 35.290 | 40.949 | 1.00 | 18.87 | C | |
| ATOM | 2719 | CG | PHE | A | 344 | 65.128 | 34.070 | 40.450 | 1.00 | 15.76 | C | |
| ATOM | 2720 | CD1 | PHE | A | 344 | 66.172 | 34.189 | 39.543 | 1.00 | 18.56 | C | |
| ATOM | 2721 | CD2 | PHE | A | 344 | 64.756 | 32.787 | 40.910 | 1.00 | 15.58 | C | |
| ATOM | 2722 | CE1 | PHE | A | 344 | 66.861 | 33.052 | 39.077 | 1.00 | 19.56 | C | |
| ATOM | 2723 | CE2 | PHE | A | 344 | 65.461 | 31.614 | 40.429 | 1.00 | 14.75 | C | |
| ATOM | 2724 | CZ | PHE | A | 344 | 66.498 | 31.775 | 39.520 | 1.00 | 17.68 | C | |
| ATOM | 2725 | N | ALA | A | 345 | 61.658 | 36.789 | 41.626 | 1.00 | 20.02 | N | |
| ATOM | 2726 | CA | ALA | A | 345 | 61.218 | 37.899 | 42.468 | 1.00 | 22.22 | C | |
| ATOM | 2727 | C | ALA | A | 345 | 62.038 | 37.792 | 43.794 | 1.00 | 22.53 | C | |
| ATOM | 2728 | O | ALA | A | 345 | 62.272 | 36.672 | 44.294 | 1.00 | 22.99 | O | |
| ATOM | 2729 | CB | ALA | A | 345 | 59.749 | 37.782 | 42.796 | 1.00 | 24.65 | C | |
| ATOM | 2730 | N | VAL | A | 346 | 62.479 | 38.925 | 44.352 | 1.00 | 22.02 | N | |
| ATOM | 2731 | CA | VAL | A | 346 | 63.244 | 38.926 | 45.628 | 1.00 | 23.78 | C | |
| ATOM | 2732 | C | VAL | A | 346 | 62.212 | 38.953 | 46.796 | 1.00 | 23.72 | C | |
| ATOM | 2733 | O | VAL | A | 346 | 61.489 | 39.944 | 46.984 | 1.00 | 23.56 | O | |
| ATOM | 2734 | CB | VAL | A | 346 | 64.201 | 40.164 | 45.703 | 1.00 | 24.25 | C | |
| ATOM | 2735 | CG1 | VAL | A | 346 | 65.054 | 40.151 | 47.010 | 1.00 | 23.26 | C | |
| ATOM | 2736 | CG2 | VAL | A | 346 | 65.132 | 40.164 | 44.508 | 1.00 | 21.14 | C | |
| ATOM | 2737 | N | LEU | A | 347 | 62.157 | 37.884 | 47.583 | 1.00 | 21.35 | N | |
| ATOM | 2738 | CA | LEU | A | 347 | 61.156 | 37.772 | 48.661 | 1.00 | 21.40 | C | |
| ATOM | 2739 | C | LEU | A | 347 | 61.445 | 38.588 | 49.902 | 1.00 | 24.60 | C | |
| ATOM | 2740 | O | LEU | A | 347 | 62.552 | 39.111 | 50.069 | 1.00 | 24.52 | O | |
| ATOM | 2741 | CB | LEU | A | 347 | 61.002 | 36.303 | 49.087 | 1.00 | 23.44 | C | |
| ATOM | 2742 | CG | LEU | A | 347 | 60.388 | 35.447 | 47.994 | 1.00 | 21.52 | C | |
| ATOM | 2743 | CD1 | LEU | A | 347 | 60.392 | 33.950 | 48.380 | 1.00 | 24.41 | C | |
| ATOM | 2744 | CD2 | LEU | A | 347 | 58.981 | 35.970 | 47.766 | 1.00 | 25.57 | C | |
| ATOM | 2745 | N | PRO | A | 348 | 60.430 | 38.719 | 50.802 | 1.00 | 24.16 | N | |
| ATOM | 2746 | CA | PRO | A | 348 | 60.631 | 39.484 | 52.041 | 1.00 | 26.26 | C | |
| ATOM | 2747 | C | PRO | A | 348 | 61.878 | 39.035 | 52.855 | 1.00 | 27.17 | C | |
| ATOM | 2748 | O | PRO | A | 348 | 62.495 | 39.884 | 53.528 | 1.00 | 27.43 | O | |
| ATOM | 2749 | CB | PRO | A | 348 | 59.303 | 39.295 | 52.794 | 1.00 | 27.70 | C | |
| ATOM | 2750 | CG | PRO | A | 348 | 58.251 | 39.230 | 51.615 | 1.00 | 28.56 | C | |
| ATOM | 2751 | CD | PRO | A | 348 | 59.012 | 38.343 | 50.603 | 1.00 | 25.60 | C | |
| ATOM | 2752 | N | ASN | A | 349 | 62.245 | 37.737 | 52.810 | 1.00 | 25.56 | N | |
| ATOM | 2753 | CA | ASN | A | 349 | 63.436 | 37.254 | 53.536 | 1.00 | 25.46 | C | |
| ATOM | 2754 | C | ASN | A | 349 | 64.694 | 37.157 | 52.630 | 1.00 | 24.57 | C | |
| ATOM | 2755 | O | ASN | A | 349 | 65.704 | 36.568 | 53.028 | 1.00 | 24.12 | O | |
| ATOM | 2756 | CB | ASN | A | 349 | 63.165 | 35.933 | 54.254 | 1.00 | 22.97 | C | |
| ATOM | 2757 | CG | ASN | A | 349 | 62.973 | 34.790 | 53.325 | 1.00 | 21.72 | C | |
| ATOM | 2758 | OD1 | ASN | A | 349 | 62.922 | 34.959 | 52.105 | 1.00 | 22.91 | O | |
| ATOM | 2759 | ND2 | ASN | A | 349 | 62.842 | 33.583 | 53.900 | 1.00 | 26.22 | N | |
| ATOM | 2760 | N | SER | A | 350 | 64.623 | 37.817 | 51.458 | 1.00 | 23.66 | N | |
| ATOM | 2761 | CA | SER | A | 350 | 65.698 | 37.934 | 50.446 | 1.00 | 22.16 | C | |
| ATOM | 2762 | C | SER | A | 350 | 65.909 | 36.680 | 49.570 | 1.00 | 20.19 | C | |
| ATOM | 2763 | O | SER | A | 350 | 66.767 | 36.661 | 48.660 | 1.00 | 20.19 | O | |
| ATOM | 2764 | CB | SER | A | 350 | 67.039 | 38.322 | 51.052 | 1.00 | 25.96 | C | |
| ATOM | 2765 | OG | SER | A | 350 | 67.665 | 37.208 | 51.668 | 1.00 | 24.38 | O | |
| ATOM | 2766 | N | ALA | A | 351 | 65.167 | 35.630 | 49.866 | 1.00 | 17.57 | N | |
| ATOM | 2767 | CA | ALA | A | 351 | 65.242 | 34.401 | 49.046 | 1.00 | 17.43 | C | |
| ATOM | 2768 | C | ALA | A | 351 | 64.694 | 34.710 | 47.628 | 1.00 | 19.61 | C | |
| ATOM | 2769 | O | ALA | A | 351 | 63.839 | 35.608 | 47.447 | 1.00 | 19.94 | O | |
| ATOM | 2770 | CB | ALA | A | 351 | 64.392 | 33.342 | 49.652 | 1.00 | 17.88 | C | |
| ATOM | 2771 | N | LEU | A | 352 | 65.127 | 33.918 | 46.652 | 1.00 | 17.19 | N | |
| ATOM | 2772 | CA | LEU | A | 352 | 64.684 | 34.149 | 45.249 | 1.00 | 18.33 | C | |
| ATOM | 2773 | C | LEU | A | 352 | 63.582 | 33.182 | 44.814 | 1.00 | 17.86 | C | |
| ATOM | 2774 | O | LEU | A | 352 | 63.717 | 31.963 | 44.881 | 1.00 | 19.93 | O | |
| ATOM | 2775 | CB | LEU | A | 352 | 65.900 | 34.050 | 44.290 | 1.00 | 16.44 | C | |
| ATOM | 2776 | CG | LEU | A | 352 | 67.075 | 34.957 | 44.640 | 1.00 | 14.50 | C | |
| ATOM | 2777 | CD1 | LEU | A | 352 | 68.215 | 34.688 | 43.723 | 1.00 | 16.06 | C | |
| ATOM | 2778 | CD2 | LEU | A | 352 | 66.636 | 36.482 | 44.580 | 1.00 | 20.65 | C | |
| ATOM | 2779 | N | ALA | A | 353 | 62.469 | 33.756 | 44.370 | 1.00 | 17.57 | N | |
| ATOM | 2780 | CA | ALA | A | 353 | 61.313 | 32.983 | 43.941 | 1.00 | 21.06 | C | |
| ATOM | 2781 | C | ALA | A | 353 | 61.305 | 32.967 | 42.412 | 1.00 | 20.69 | C | |
| ATOM | 2782 | O | ALA | A | 353 | 61.239 | 34.015 | 41.806 | 1.00 | 20.98 | O | |
| ATOM | 2783 | CB | ALA | A | 353 | 60.009 | 33.647 | 44.465 | 1.00 | 18.87 | C | |
| ATOM | 2784 | N | SER | A | 354 | 61.398 | 31.784 | 41.813 | 1.00 | 19.36 | N | |
| ATOM | 2785 | CA | SER | A | 354 | 61.416 | 31.692 | 40.356 | 1.00 | 19.68 | C | |
| ATOM | 2786 | C | SER | A | 354 | 60.084 | 32.128 | 39.696 | 1.00 | 19.49 | C | |
| ATOM | 2787 | O | SER | A | 354 | 59.032 | 31.694 | 40.121 | 1.00 | 22.59 | O | |
| ATOM | 2788 | CB | SER | A | 354 | 61.690 | 30.256 | 39.912 | 1.00 | 23.90 | C | |
| ATOM | 2789 | OG | SER | A | 354 | 61.782 | 30.228 | 38.479 | 1.00 | 23.89 | O | |
| ATOM | 2790 | N | LEU | A | 355 | 60.164 | 32.993 | 38.670 | 1.00 | 20.43 | N | |
| ATOM | 2791 | CA | LEU | A | 355 | 58.960 | 33.445 | 37.928 | 1.00 | 20.07 | C | |
| ATOM | 2792 | C | LEU | A | 355 | 58.822 | 32.665 | 36.626 | 1.00 | 21.96 | C | |
| ATOM | 2793 | O | LEU | A | 355 | 57.739 | 32.156 | 36.303 | 1.00 | 24.19 | O | |
| ATOM | 2794 | CB | LEU | A | 355 | 59.043 | 34.945 | 37.638 | 1.00 | 21.68 | C | |
| ATOM | 2795 | CG | LEU | A | 355 | 59.206 | 35.858 | 38.849 | 1.00 | 22.80 | C | |
| ATOM | 2796 | CD1 | LEU | A | 355 | 59.478 | 37.260 | 38.383 | 1.00 | 23.43 | C | |
| ATOM | 2797 | CD2 | LEU | A | 355 | 57.921 | 35.798 | 39.749 | 1.00 | 25.12 | C | |
| ATOM | 2798 | N | PHE | A | 356 | 59.898 | 32.592 | 35.841 | 1.00 | 19.57 | N | |
| ATOM | 2799 | CA | PHE | A | 356 | 59.899 | 31.840 | 34.581 | 1.00 | 20.80 | C | |
| ATOM | 2800 | C | PHE | A | 356 | 61.344 | 31.567 | 34.138 | 1.00 | 18.63 | C | |
| ATOM | 2801 | O | PHE | A | 356 | 62.292 | 32.207 | 34.593 | 1.00 | 19.60 | O | |
| ATOM | 2802 | CB | PHE | A | 356 | 59.126 | 32.614 | 33.457 | 1.00 | 20.93 | C | |
| ATOM | 2803 | CG | PHE | A | 356 | 59.673 | 34.009 | 33.161 | 1.00 | 23.84 | C | |
| ATOM | 2804 | CD1 | PHE | A | 356 | 60.858 | 34.182 | 32.475 | 1.00 | 26.64 | C | |
| ATOM | 2805 | CD2 | PHE | A | 356 | 58.989 | 35.140 | 33.604 | 1.00 | 24.03 | C | |
| ATOM | 2806 | CE1 | PHE | A | 356 | 61.375 | 35.455 | 32.229 | 1.00 | 27.67 | C | |
| ATOM | 2807 | CE2 | PHE | A | 356 | 59.489 | 36.421 | 33.370 | 1.00 | 25.15 | C | |
| ATOM | 2808 | CZ | PHE | A | 356 | 60.671 | 36.589 | 32.695 | 1.00 | 26.42 | C | |
| ATOM | 2809 | N | LEU | A | 357 | 61.478 | 30.592 | 33.251 | 1.00 | 19.79 | N | |
| ATOM | 2810 | CA | LEU | A | 357 | 62.752 | 30.169 | 32.675 | 1.00 | 21.15 | C | |
| ATOM | 2811 | C | LEU | A | 357 | 62.615 | 30.046 | 31.150 | 1.00 | 21.32 | C | |
| ATOM | 2812 | O | LEU | A | 357 | 61.776 | 29.289 | 30.654 | 1.00 | 20.75 | O | |
| ATOM | 2813 | CB | LEU | A | 357 | 63.133 | 28.791 | 33.242 | 1.00 | 22.34 | C | |
| ATOM | 2814 | CG | LEU | A | 357 | 64.325 | 28.066 | 32.605 | 1.00 | 27.32 | C | |
| ATOM | 2815 | CD1 | LEU | A | 357 | 65.622 | 28.735 | 32.999 | 1.00 | 25.79 | C | |
| ATOM | 2816 | CD2 | LEU | A | 357 | 64.340 | 26.586 | 33.104 | 1.00 | 29.65 | C | |
| ATOM | 2817 | N | ILE | A | 358 | 63.457 | 30.770 | 30.409 | 1.00 | 21.21 | N | |
| ATOM | 2818 | CA | ILE | A | 358 | 63.431 | 30.670 | 28.934 | 1.00 | 21.64 | C | |
| ATOM | 2819 | C | ILE | A | 358 | 64.804 | 30.271 | 28.415 | 1.00 | 21.87 | C | |
| ATOM | 2820 | O | ILE | A | 358 | 65.807 | 30.386 | 29.126 | 1.00 | 22.77 | O | |
| ATOM | 2821 | CB | ILE | A | 358 | 63.073 | 32.030 | 28.264 | 1.00 | 20.59 | C | |
| ATOM | 2822 | CG1 | ILE | A | 358 | 64.158 | 33.089 | 28.505 | 1.00 | 24.28 | C | |
| ATOM | 2823 | CG2 | ILE | A | 358 | 61.794 | 32.530 | 28.849 | 1.00 | 19.95 | C | |
| ATOM | 2824 | CD1 | ILE | A | 358 | 63.879 | 34.389 | 27.723 | 1.00 | 26.29 | C | |
| ATOM | 2825 | N | GLY | A | 359 | 64.835 | 29.816 | 27.168 | 1.00 | 20.62 | N | |
| ATOM | 2826 | CA | GLY | A | 359 | 66.086 | 29.455 | 26.500 | 1.00 | 20.37 | C | |
| ATOM | 2827 | C | GLY | A | 359 | 66.262 | 30.501 | 25.401 | 1.00 | 23.47 | C | |
| ATOM | 2828 | O | GLY | A | 359 | 65.265 | 30.938 | 24.788 | 1.00 | 22.13 | O | |
| ATOM | 2829 | N | MET | A | 360 | 67.497 | 30.920 | 25.168 | 1.00 | 20.48 | N | |
| ATOM | 2830 | CA | MET | A | 360 | 67.765 | 31.937 | 24.169 | 1.00 | 24.38 | C | |
| ATOM | 2831 | C | MET | A | 360 | 68.943 | 31.537 | 23.330 | 1.00 | 25.27 | C | |
| ATOM | 2832 | O | MET | A | 360 | 69.973 | 31.112 | 23.866 | 1.00 | 25.29 | O | |
| ATOM | 2833 | CB | MET | A | 360 | 68.066 | 33.291 | 24.835 | 1.00 | 24.51 | C | |
| ATOM | 2834 | CG | MET | A | 360 | 68.499 | 34.363 | 23.905 | 1.00 | 24.56 | C | |
| ATOM | 2835 | SD | MET | A | 360 | 68.539 | 35.980 | 24.800 | 1.00 | 34.34 | S | |
| ATOM | 2836 | CE | MET | A | 360 | 69.928 | 35.750 | 25.943 | 1.00 | 30.52 | C | |
| ATOM | 2837 | N | HIS | A | 361 | 68.797 | 31.605 | 22.005 | 1.00 | 22.34 | N | |
| ATOM | 2838 | CA | HIS | A | 361 | 69.962 | 31.266 | 21.198 | 1.00 | 23.47 | C | |
| ATOM | 2839 | C | HIS | A | 361 | 70.030 | 32.059 | 19.915 | 1.00 | 20.15 | C | |
| ATOM | 2840 | O | HIS | A | 361 | 69.053 | 32.667 | 19.491 | 1.00 | 19.89 | O | |
| ATOM | 2841 | CB | HIS | A | 361 | 70.037 | 29.772 | 20.864 | 1.00 | 26.13 | C | |
| ATOM | 2842 | CG | HIS | A | 361 | 69.024 | 29.334 | 19.860 | 1.00 | 28.93 | C | |
| ATOM | 2843 | ND1 | HIS | A | 361 | 67.695 | 29.165 | 20.174 | 1.00 | 33.10 | N | |
| ATOM | 2844 | CD2 | HIS | A | 361 | 69.140 | 29.055 | 18.542 | 1.00 | 32.51 | C | |
| ATOM | 2845 | CE1 | HIS | A | 361 | 67.029 | 28.802 | 19.086 | 1.00 | 33.63 | C | |
| ATOM | 2846 | NE2 | HIS | A | 361 | 67.884 | 28.732 | 18.083 | 1.00 | 34.52 | N | |
| ATOM | 2847 | N | THR | A | 362 | 71.213 | 32.049 | 19.322 | 1.00 | 19.48 | N | |
| ATOM | 2848 | CA | THR | A | 362 | 71.428 | 32.776 | 18.090 | 1.00 | 19.69 | C | |
| ATOM | 2849 | C | THR | A | 362 | 72.731 | 32.299 | 17.443 | 1.00 | 20.11 | C | |
| ATOM | 2850 | O | THR | A | 362 | 73.406 | 31.328 | 17.947 | 1.00 | 21.70 | O | |
| ATOM | 2851 | CB | THR | A | 362 | 71.480 | 34.323 | 18.404 | 1.00 | 22.62 | C | |
| ATOM | 2852 | OG1 | THR | A | 362 | 71.371 | 35.056 | 17.175 | 1.00 | 27.64 | O | |
| ATOM | 2853 | CG2 | THR | A | 362 | 72.815 | 34.703 | 19.095 | 1.00 | 26.71 | C | |
| ATOM | 2854 | N | ATHR | A | 363 | 73.064 | 32.898 | 16.305 | 0.50 | 18.46 | N | |
| ATOM | 2855 | N | BTHR | A | 363 | 73.060 | 32.901 | 16.308 | 0.50 | 19.24 | N | |
| ATOM | 2856 | CA | ATHR | A | 363 | 74.303 | 32.605 | 15.608 | 0.50 | 19.66 | C | |
| ATOM | 2857 | CA | BTHR | A | 363 | 74.297 | 32.620 | 15.606 | 0.50 | 21.08 | C | |
| ATOM | 2858 | C | ATHR | A | 363 | 75.084 | 33.914 | 15.691 | 0.50 | 22.23 | C | |
| ATOM | 2859 | C | BTHR | A | 363 | 75.085 | 33.923 | 15.683 | 0.50 | 22.94 | C | |
| ATOM | 2860 | O | ATHR | A | 363 | 74.496 | 34.998 | 15.696 | 0.50 | 23.41 | O | |
| ATOM | 2861 | O | BTHR | A | 363 | 74.508 | 35.012 | 15.666 | 0.50 | 24.03 | O | |
| ATOM | 2862 | CB | ATHR | A | 363 | 74.071 | 32.245 | 14.120 | 0.50 | 21.19 | C | |
| ATOM | 2863 | CB | BTHR | A | 363 | 74.063 | 32.269 | 14.125 | 0.50 | 23.38 | C | |
| ATOM | 2864 | OG1 | ATHR | A | 363 | 73.184 | 31.126 | 14.042 | 0.50 | 22.34 | O | |
| ATOM | 2865 | OG1 | BTHR | A | 363 | 73.045 | 33.121 | 13.585 | 0.50 | 23.47 | O | |
| ATOM | 2866 | CG2 | ATHR | A | 363 | 75.388 | 31.880 | 13.433 | 0.50 | 16.25 | C | |
| ATOM | 2867 | CG2 | BTHR | A | 363 | 73.647 | 30.825 | 13.986 | 0.50 | 23.11 | C | |
| ATOM | 2868 | N | GLY | A | 364 | 76.396 | 33.820 | 15.781 | 1.00 | 21.74 | N | |
| ATOM | 2869 | CA | GLY | A | 364 | 77.186 | 35.032 | 15.851 | 1.00 | 22.62 | C | |
| ATOM | 2870 | C | GLY | A | 364 | 78.452 | 34.892 | 15.044 | 1.00 | 24.44 | C | |
| ATOM | 2871 | O | GLY | A | 364 | 78.956 | 33.787 | 14.843 | 1.00 | 25.40 | O | |
| ATOM | 2872 | N | SER | A | 365 | 78.945 | 36.003 | 14.527 | 1.00 | 23.38 | N | |
| ATOM | 2873 | CA | SER | A | 365 | 80.192 | 35.942 | 13.792 | 1.00 | 22.86 | C | |
| ATOM | 2874 | C | SER | A | 365 | 81.290 | 36.241 | 14.802 | 1.00 | 24.57 | C | |
| ATOM | 2875 | O | SER | A | 365 | 81.112 | 37.082 | 15.703 | 1.00 | 23.77 | O | |
| ATOM | 2876 | CB | SER | A | 365 | 80.218 | 37.003 | 12.689 | 1.00 | 24.97 | C | |
| ATOM | 2877 | OG | SER | A | 365 | 79.977 | 38.296 | 13.224 | 1.00 | 28.94 | O | |
| ATOM | 2878 | N | MET | A | 366 | 82.426 | 35.565 | 14.659 | 1.00 | 21.45 | N | |
| ATOM | 2879 | CA | MET | A | 366 | 83.558 | 35.814 | 15.541 | 1.00 | 23.58 | C | |
| ATOM | 2880 | C | MET | A | 366 | 84.711 | 36.287 | 14.663 | 1.00 | 22.96 | C | |
| ATOM | 2881 | O | MET | A | 366 | 85.320 | 35.489 | 13.940 | 1.00 | 24.69 | O | |
| ATOM | 2882 | CB | MET | A | 366 | 83.958 | 34.520 | 16.251 | 1.00 | 22.84 | C | |
| ATOM | 2883 | CG | MET | A | 366 | 85.057 | 34.728 | 17.271 | 1.00 | 26.18 | C | |
| ATOM | 2884 | SD | MET | A | 366 | 84.540 | 35.601 | 18.741 | 1.00 | 30.64 | S | |
| ATOM | 2885 | CE | MET | A | 366 | 83.536 | 34.348 | 19.608 | 1.00 | 29.02 | C | |
| ATOM | 2886 | N | GLU | A | 367 | 84.989 | 37.582 | 14.684 | 1.00 | 22.33 | N | |
| ATOM | 2887 | CA | GLU | A | 367 | 86.101 | 38.136 | 13.896 | 1.00 | 22.19 | C | |
| ATOM | 2888 | C | GLU | A | 367 | 87.358 | 37.956 | 14.738 | 1.00 | 22.50 | C | |
| ATOM | 2889 | O | GLU | A | 367 | 87.382 | 38.407 | 15.863 | 1.00 | 24.63 | O | |
| ATOM | 2890 | CB | GLU | A | 367 | 85.895 | 39.626 | 13.660 | 1.00 | 25.42 | C | |
| ATOM | 2891 | CG | GLU | A | 367 | 84.694 | 39.934 | 12.758 | 1.00 | 37.35 | C | |
| ATOM | 2892 | CD | GLU | A | 367 | 84.340 | 41.418 | 12.752 | 1.00 | 43.75 | C | |
| ATOM | 2893 | OE1 | GLU | A | 367 | 83.566 | 41.865 | 13.642 | 1.00 | 44.58 | O | |
| ATOM | 2894 | OE2 | GLU | A | 367 | 84.852 | 42.142 | 11.860 | 1.00 | 47.82 | O | |
| ATOM | 2895 | N | VAL | A | 368 | 88.390 | 37.321 | 14.192 | 1.00 | 25.43 | N | |
| ATOM | 2896 | CA | VAL | A | 368 | 89.637 | 37.032 | 14.921 | 1.00 | 25.46 | C | |
| ATOM | 2897 | C | VAL | A | 368 | 90.826 | 37.811 | 14.325 | 1.00 | 31.07 | C | |
| ATOM | 2898 | O | VAL | A | 368 | 91.007 | 37.853 | 13.087 | 1.00 | 33.39 | O | |
| ATOM | 2899 | CB | VAL | A | 368 | 89.922 | 35.497 | 14.846 | 1.00 | 27.14 | C | |
| ATOM | 2900 | CG1 | VAL | A | 368 | 91.264 | 35.146 | 15.485 | 1.00 | 27.94 | C | |
| ATOM | 2901 | CG2 | VAL | A | 368 | 88.843 | 34.752 | 15.573 | 1.00 | 27.31 | C | |
| ATOM | 2902 | N | SER | A | 369 | 91.635 | 38.413 | 15.198 | 1.00 | 29.11 | N | |
| ATOM | 2903 | CA | SER | A | 369 | 92.799 | 39.176 | 14.777 | 1.00 | 29.86 | C | |
| ATOM | 2904 | C | SER | A | 369 | 94.057 | 38.867 | 15.598 | 1.00 | 29.30 | C | |
| ATOM | 2905 | O | SER | A | 369 | 94.040 | 38.013 | 16.486 | 1.00 | 22.65 | O | |
| ATOM | 2906 | CB | SER | A | 369 | 92.478 | 40.674 | 14.868 | 1.00 | 34.13 | C | |
| ATOM | 2907 | OG | SER | A | 369 | 93.670 | 41.424 | 14.968 | 1.00 | 42.36 | O | |
| ATOM | 2908 | N | ALA | A | 370 | 95.157 | 39.571 | 15.285 | 1.00 | 34.69 | N | |
| ATOM | 2909 | CA | ALA | A | 370 | 96.456 | 39.405 | 16.000 | 1.00 | 36.73 | C | |
| ATOM | 2910 | C | ALA | A | 370 | 96.831 | 40.725 | 16.642 | 1.00 | 38.68 | C | |
| ATOM | 2911 | O | ALA | A | 370 | 96.547 | 41.804 | 16.106 | 1.00 | 39.76 | O | |
| ATOM | 2912 | CB | ALA | A | 370 | 97.582 | 38.981 | 15.044 | 1.00 | 32.66 | C | |
| ATOM | 2913 | N | GLU | A | 371 | 97.508 | 40.638 | 17.771 | 1.00 | 38.55 | N | |
| ATOM | 2914 | CA | GLU | A | 371 | 97.875 | 41.833 | 18.485 | 1.00 | 39.00 | C | |
| ATOM | 2915 | C | GLU | A | 371 | 98.885 | 41.424 | 19.528 | 1.00 | 38.33 | C | |
| ATOM | 2916 | O | GLU | A | 371 | 98.551 | 40.729 | 20.485 | 1.00 | 39.69 | O | |
| ATOM | 2917 | CB | GLU | A | 371 | 96.626 | 42.421 | 19.162 | 1.00 | 43.20 | C | |
| ATOM | 2918 | CG | GLU | A | 371 | 96.333 | 43.851 | 18.798 | 1.00 | 48.61 | C | |
| ATOM | 2919 | CD | GLU | A | 371 | 97.057 | 44.816 | 19.700 | 1.00 | 51.48 | C | |
| ATOM | 2920 | OE1 | GLU | A | 371 | 96.487 | 45.190 | 20.758 | 1.00 | 48.95 | O | |
| ATOM | 2921 | OE2 | GLU | A | 371 | 98.196 | 45.175 | 19.344 | 1.00 | 50.85 | O | |
| ATOM | 2922 | N | SER | A | 372 | 100.132 | 41.821 | 19.314 | 1.00 | 35.49 | N | |
| ATOM | 2923 | CA | SER | A | 372 | 101.190 | 41.511 | 20.248 | 1.00 | 31.31 | C | |
| ATOM | 2924 | C | SER | A | 372 | 101.323 | 40.052 | 20.641 | 1.00 | 27.35 | C | |
| ATOM | 2925 | O | SER | A | 372 | 101.291 | 39.766 | 21.846 | 1.00 | 27.78 | O | |
| ATOM | 2926 | CB | SER | A | 372 | 101.044 | 42.347 | 21.541 | 1.00 | 37.05 | C | |
| ATOM | 2927 | OG | SER | A | 372 | 101.101 | 43.747 | 21.281 | 1.00 | 43.04 | O | |
| ATOM | 2928 | N | AASN | A | 373 | 101.490 | 39.166 | 19.651 | 0.50 | 23.03 | N | |
| ATOM | 2929 | N | BASN | A | 373 | 101.462 | 39.128 | 19.684 | 0.50 | 21.92 | N | |
| ATOM | 2930 | CA | AASN | A | 373 | 101.663 | 37.736 | 19.885 | 0.50 | 20.77 | C | |
| ATOM | 2931 | CA | BASN | A | 373 | 101.638 | 37.722 | 20.052 | 0.50 | 18.76 | C | |
| ATOM | 2932 | C | AASN | A | 373 | 100.508 | 37.084 | 20.656 | 0.50 | 21.04 | C | |
| ATOM | 2933 | C | BASN | A | 373 | 100.435 | 37.083 | 20.742 | 0.50 | 19.62 | C | |
| ATOM | 2934 | O | AASN | A | 373 | 100.698 | 36.158 | 21.447 | 0.50 | 21.27 | O | |
| ATOM | 2935 | O | BASN | A | 373 | 100.566 | 36.140 | 21.532 | 0.50 | 18.66 | O | |
| ATOM | 2936 | CB | AASN | A | 373 | 103.031 | 37.472 | 20.566 | 0.50 | 21.57 | C | |
| ATOM | 2937 | CB | BASN | A | 373 | 102.874 | 37.567 | 20.954 | 0.50 | 17.20 | C | |
| ATOM | 2938 | CG | AASN | A | 373 | 103.293 | 38.381 | 21.734 | 0.50 | 18.77 | C | |
| ATOM | 2939 | CG | BASN | A | 373 | 104.171 | 37.692 | 20.179 | 0.50 | 14.45 | C | |
| ATOM | 2940 | OD1 | AASN | A | 373 | 104.363 | 39.016 | 21.852 | 0.50 | 27.43 | O | |
| ATOM | 2941 | OD1 | BASN | A | 373 | 105.262 | 37.518 | 20.735 | 0.50 | 23.77 | O | |
| ATOM | 2942 | ND2 | AASN | A | 373 | 102.338 | 38.458 | 22.607 | 0.50 | 24.72 | N | |
| ATOM | 2943 | ND2 | BASN | A | 373 | 104.064 | 37.995 | 18.883 | 0.50 | 18.75 | N | |
| ATOM | 2944 | N | AARG | A | 374 | 99.298 | 37.593 | 20.445 | 0.50 | 21.03 | N | |
| ATOM | 2945 | N | BARG | A | 374 | 99.252 | 37.617 | 20.497 | 0.50 | 20.06 | N | |
| ATOM | 2946 | CA | AARG | A | 374 | 98.121 | 37.002 | 21.069 | 0.50 | 21.58 | C | |
| ATOM | 2947 | CA | BARG | A | 374 | 98.082 | 36.998 | 21.079 | 0.50 | 21.50 | C | |
| ATOM | 2948 | C | AARG | A | 374 | 97.011 | 37.029 | 20.022 | 0.50 | 20.79 | C | |
| ATOM | 2949 | C | BARG | A | 374 | 96.991 | 37.046 | 20.034 | 0.50 | 20.80 | C | |
| ATOM | 2950 | O | AARG | A | 374 | 97.118 | 37.771 | 19.036 | 0.50 | 21.98 | O | |
| ATOM | 2951 | O | BARG | A | 374 | 97.089 | 37.807 | 19.060 | 0.50 | 22.34 | O | |
| ATOM | 2952 | CB | AARG | A | 374 | 97.713 | 37.766 | 22.345 | 0.50 | 25.48 | C | |
| ATOM | 2953 | CB | BARG | A | 374 | 97.663 | 37.702 | 22.380 | 0.50 | 25.76 | C | |
| ATOM | 2954 | CG | AARG | A | 374 | 97.013 | 39.119 | 22.200 | 0.50 | 29.02 | C | |
| ATOM | 2955 | CG | BARG | A | 374 | 97.251 | 39.149 | 22.271 | 0.50 | 30.43 | C | |
| ATOM | 2956 | CD | AARG | A | 374 | 96.949 | 39.817 | 23.608 | 0.50 | 30.65 | C | |
| ATOM | 2957 | CD | BARG | A | 374 | 97.481 | 39.907 | 23.615 | 0.50 | 30.79 | C | |
| ATOM | 2958 | NE | AARG | A | 374 | 96.045 | 40.973 | 23.693 | 0.50 | 34.22 | N | |
| ATOM | 2959 | NE | BARG | A | 374 | 98.908 | 40.082 | 23.892 | 0.50 | 32.09 | N | |
| ATOM | 2960 | CZ | AARG | A | 374 | 96.305 | 42.197 | 23.238 | 0.50 | 34.15 | C | |
| ATOM | 2961 | CZ | BARG | A | 374 | 99.409 | 40.778 | 24.910 | 0.50 | 33.07 | C | |
| ATOM | 2962 | NH1 | AARG | A | 374 | 97.446 | 42.479 | 22.637 | 0.50 | 35.34 | N | |
| ATOM | 2963 | NH1 | BARG | A | 374 | 98.604 | 41.384 | 25.773 | 0.50 | 35.29 | N | |
| ATOM | 2964 | NH2 | AARG | A | 374 | 95.424 | 43.163 | 23.420 | 0.50 | 37.32 | N | |
| ATOM | 2965 | NH2 | BARG | A | 374 | 100.718 | 40.866 | 25.076 | 0.50 | 31.01 | N | |
| ATOM | 2966 | N | LEU | A | 375 | 95.993 | 36.190 | 20.213 | 1.00 | 20.17 | N | |
| ATOM | 2967 | CA | LEU | A | 375 | 94.836 | 36.132 | 19.300 | 1.00 | 20.90 | C | |
| ATOM | 2968 | C | LEU | A | 375 | 93.726 | 36.900 | 20.015 | 1.00 | 18.90 | C | |
| ATOM | 2969 | O | LEU | A | 375 | 93.488 | 36.679 | 21.200 | 1.00 | 18.99 | O | |
| ATOM | 2970 | CB | LEU | A | 375 | 94.349 | 34.671 | 19.149 | 1.00 | 23.32 | C | |
| ATOM | 2971 | CG | LEU | A | 375 | 93.360 | 34.539 | 17.996 | 1.00 | 33.86 | C | |
| ATOM | 2972 | CD1 | LEU | A | 375 | 94.113 | 34.782 | 16.677 | 1.00 | 37.49 | C | |
| ATOM | 2973 | CD2 | LEU | A | 375 | 92.707 | 33.122 | 18.017 | 1.00 | 36.55 | C | |
| ATOM | 2974 | N | VAL | A | 376 | 93.026 | 37.784 | 19.318 | 1.00 | 17.78 | N | |
| ATOM | 2975 | CA | VAL | A | 376 | 91.949 | 38.535 | 19.977 | 1.00 | 17.47 | C | |
| ATOM | 2976 | C | VAL | A | 376 | 90.673 | 38.352 | 19.122 | 1.00 | 22.75 | C | |
| ATOM | 2977 | O | VAL | A | 376 | 90.747 | 38.096 | 17.909 | 1.00 | 20.82 | O | |
| ATOM | 2978 | CB | VAL | A | 376 | 92.338 | 40.044 | 20.116 | 1.00 | 23.91 | C | |
| ATOM | 2979 | CG1 | VAL | A | 376 | 93.604 | 40.142 | 20.958 | 1.00 | 23.51 | C | |
| ATOM | 2980 | CG2 | VAL | A | 376 | 92.589 | 40.661 | 18.800 | 1.00 | 23.25 | C | |
| ATOM | 2981 | N | GLY | A | 377 | 89.516 | 38.467 | 19.770 | 1.00 | 20.65 | N | |
| ATOM | 2982 | CA | GLY | A | 377 | 88.263 | 38.290 | 19.048 | 1.00 | 20.98 | C | |
| ATOM | 2983 | C | GLY | A | 377 | 87.196 | 39.353 | 19.299 | 1.00 | 20.73 | C | |
| ATOM | 2984 | O | GLY | A | 377 | 87.263 | 40.094 | 20.292 | 1.00 | 21.26 | O | |
| ATOM | 2985 | N | GLU | A | 378 | 86.192 | 39.388 | 18.423 | 1.00 | 19.36 | N | |
| ATOM | 2986 | CA | GLU | A | 378 | 85.057 | 40.313 | 18.583 | 1.00 | 19.80 | C | |
| ATOM | 2987 | C | GLU | A | 378 | 83.804 | 39.536 | 18.086 | 1.00 | 19.25 | C | |
| ATOM | 2988 | O | GLU | A | 378 | 83.764 | 39.095 | 16.949 | 1.00 | 20.85 | O | |
| ATOM | 2989 | CB | GLU | A | 378 | 85.312 | 41.600 | 17.753 | 1.00 | 20.50 | C | |
| ATOM | 2990 | CG | GLU | A | 378 | 84.156 | 42.571 | 17.702 | 1.00 | 26.76 | C | |
| ATOM | 2991 | CD | GLU | A | 378 | 84.022 | 43.399 | 18.943 | 1.00 | 32.24 | C | |
| ATOM | 2992 | OE1 | GLU | A | 378 | 83.020 | 44.153 | 19.038 | 1.00 | 38.76 | O | |
| ATOM | 2993 | OE2 | GLU | A | 378 | 84.936 | 43.325 | 19.815 | 1.00 | 35.19 | O | |
| ATOM | 2994 | N | LEU | A | 379 | 82.809 | 39.382 | 18.971 | 1.00 | 18.13 | N | |
| ATOM | 2995 | CA | LEU | A | 379 | 81.568 | 38.633 | 18.694 | 1.00 | 18.29 | C | |
| ATOM | 2996 | C | LEU | A | 379 | 80.430 | 39.596 | 18.361 | 1.00 | 20.04 | C | |
| ATOM | 2997 | O | LEU | A | 379 | 80.247 | 40.584 | 19.087 | 1.00 | 18.78 | O | |
| ATOM | 2998 | CB | LEU | A | 379 | 81.174 | 37.869 | 19.977 | 1.00 | 17.99 | C | |
| ATOM | 2999 | CG | LEU | A | 379 | 80.306 | 36.607 | 19.972 | 1.00 | 29.39 | C | |
| ATOM | 3000 | CD1 | LEU | A | 379 | 79.402 | 36.568 | 21.181 | 1.00 | 23.12 | C | |
| ATOM | 3001 | CD2 | LEU | A | 379 | 79.631 | 36.414 | 18.654 | 1.00 | 23.47 | C | |
| ATOM | 3002 | N | LYS | A | 380 | 79.698 | 39.323 | 17.260 | 1.00 | 20.00 | N | |
| ATOM | 3003 | CA | LYS | A | 380 | 78.526 | 40.106 | 16.874 | 1.00 | 20.24 | C | |
| ATOM | 3004 | C | LYS | A | 380 | 77.417 | 39.134 | 16.653 | 1.00 | 19.64 | C | |
| ATOM | 3005 | O | LYS | A | 380 | 77.629 | 38.088 | 16.020 | 1.00 | 23.51 | O | |
| ATOM | 3006 | CB | LYS | A | 380 | 78.732 | 40.894 | 15.570 | 1.00 | 26.90 | C | |
| ATOM | 3007 | CG | LYS | A | 380 | 79.750 | 41.979 | 15.700 | 1.00 | 32.63 | C | |
| ATOM | 3008 | CD | LYS | A | 380 | 81.136 | 41.376 | 15.914 | 1.00 | 36.57 | C | |
| ATOM | 3009 | CE | LYS | A | 380 | 81.237 | 40.018 | 15.216 | 1.00 | 37.63 | C | |
| ATOM | 3010 | NZ | LYS | A | 380 | 82.402 | 39.807 | 14.450 | 1.00 | 33.27 | N | |
| ATOM | 3011 | N | LEU | A | 381 | 76.236 | 39.517 | 17.103 | 1.00 | 18.63 | N | |
| ATOM | 3012 | CA | LEU | A | 381 | 75.099 | 38.607 | 17.098 | 1.00 | 19.16 | C | |
| ATOM | 3013 | C | LEU | A | 381 | 73.972 | 38.789 | 16.105 | 1.00 | 21.37 | C | |
| ATOM | 3014 | O | LEU | A | 381 | 73.656 | 39.899 | 15.714 | 1.00 | 20.52 | O | |
| ATOM | 3015 | CB | LEU | A | 381 | 74.476 | 38.644 | 18.464 | 1.00 | 21.98 | C | |
| ATOM | 3016 | CG | LEU | A | 381 | 75.389 | 38.382 | 19.658 | 1.00 | 24.30 | C | |
| ATOM | 3017 | CD1 | LEU | A | 381 | 74.628 | 38.644 | 20.965 | 1.00 | 26.03 | C | |
| ATOM | 3018 | CD2 | LEU | A | 381 | 75.902 | 36.946 | 19.581 | 1.00 | 23.45 | C | |
| ATOM | 3019 | N | ASP | A | 382 | 73.357 | 37.687 | 15.715 | 1.00 | 17.92 | N | |
| ATOM | 3020 | CA | ASP | A | 382 | 72.167 | 37.792 | 14.856 | 1.00 | 20.04 | C | |
| ATOM | 3021 | C | ASP | A | 382 | 70.918 | 37.926 | 15.757 | 1.00 | 21.87 | C | |
| ATOM | 3022 | O | ASP | A | 382 | 71.002 | 38.201 | 16.959 | 1.00 | 22.38 | O | |
| ATOM | 3023 | CB | ASP | A | 382 | 72.013 | 36.589 | 13.917 | 1.00 | 23.47 | C | |
| ATOM | 3024 | CG | ASP | A | 382 | 73.123 | 36.502 | 12.867 | 1.00 | 29.81 | C | |
| ATOM | 3025 | OD1 | ASP | A | 382 | 73.660 | 37.553 | 12.445 | 1.00 | 36.54 | O | |
| ATOM | 3026 | OD2 | ASP | A | 382 | 73.460 | 35.383 | 12.423 | 1.00 | 31.44 | O | |
| ATOM | 3027 | N | ARG | A | 383 | 69.725 | 37.747 | 15.180 | 1.00 | 24.00 | N | |
| ATOM | 3028 | CA | ARG | A | 383 | 68.482 | 37.884 | 15.928 | 1.00 | 23.87 | C | |
| ATOM | 3029 | C | ARG | A | 383 | 68.398 | 36.894 | 17.094 | 1.00 | 23.10 | C | |
| ATOM | 3030 | O | ARG | A | 383 | 68.840 | 35.752 | 16.952 | 1.00 | 24.99 | O | |
| ATOM | 3031 | CB | ARG | A | 383 | 67.309 | 37.648 | 14.962 | 1.00 | 28.83 | C | |
| ATOM | 3032 | CG | ARG | A | 383 | 66.047 | 38.259 | 15.397 | 1.00 | 34.77 | C | |
| ATOM | 3033 | CD | ARG | A | 383 | 65.028 | 38.203 | 14.249 | 1.00 | 39.89 | C | |
| ATOM | 3034 | NE | ARG | A | 383 | 63.924 | 37.293 | 14.557 | 1.00 | 50.79 | N | |
| ATOM | 3035 | CZ | ARG | A | 383 | 63.619 | 36.201 | 13.854 | 1.00 | 52.55 | C | |
| ATOM | 3036 | NH1 | ARG | A | 383 | 64.333 | 35.864 | 12.781 | 1.00 | 54.41 | N | |
| ATOM | 3037 | NH2 | ARG | A | 383 | 62.605 | 35.437 | 14.238 | 1.00 | 54.44 | N | |
| ATOM | 3038 | N | LEU | A | 384 | 67.846 | 37.337 | 18.228 | 1.00 | 23.74 | N | |
| ATOM | 3039 | CA | LEU | A | 384 | 67.732 | 36.491 | 19.410 | 1.00 | 23.25 | C | |
| ATOM | 3040 | C | LEU | A | 384 | 66.470 | 35.649 | 19.340 | 1.00 | 23.45 | C | |
| ATOM | 3041 | O | LEU | A | 384 | 65.358 | 36.174 | 19.247 | 1.00 | 25.21 | O | |
| ATOM | 3042 | CB | LEU | A | 384 | 67.768 | 37.349 | 20.705 | 1.00 | 22.74 | C | |
| ATOM | 3043 | CG | LEU | A | 384 | 68.963 | 38.299 | 20.801 | 1.00 | 22.83 | C | |
| ATOM | 3044 | CD1 | LEU | A | 384 | 68.867 | 39.112 | 22.132 | 1.00 | 25.00 | C | |
| ATOM | 3045 | CD2 | LEU | A | 384 | 70.293 | 37.502 | 20.742 | 1.00 | 24.54 | C | |
| ATOM | 3046 | N | LEU | A | 385 | 66.650 | 34.330 | 19.384 | 1.00 | 22.53 | N | |
| ATOM | 3047 | CA | LEU | A | 385 | 65.505 | 33.400 | 19.324 | 1.00 | 24.05 | C | |
| ATOM | 3048 | C | LEU | A | 385 | 65.171 | 32.872 | 20.711 | 1.00 | 23.35 | C | |
| ATOM | 3049 | O | LEU | A | 385 | 66.019 | 32.238 | 21.346 | 1.00 | 24.19 | O | |
| ATOM | 3050 | CB | LEU | A | 385 | 65.823 | 32.252 | 18.378 | 1.00 | 20.91 | C | |
| ATOM | 3051 | CG | LEU | A | 385 | 66.216 | 32.659 | 16.923 | 1.00 | 26.56 | C | |
| ATOM | 3052 | CD1 | LEU | A | 385 | 66.668 | 31.412 | 16.157 | 1.00 | 25.11 | C | |
| ATOM | 3053 | CD2 | LEU | A | 385 | 65.008 | 33.327 | 16.202 | 1.00 | 28.62 | C | |
| ATOM | 3054 | N | LEU | A | 386 | 63.926 | 33.091 | 21.146 | 1.00 | 21.44 | N | |
| ATOM | 3055 | CA | LEU | A | 386 | 63.493 | 32.727 | 22.507 | 1.00 | 22.95 | C | |
| ATOM | 3056 | C | LEU | A | 386 | 62.544 | 31.537 | 22.531 | 1.00 | 24.92 | C | |
| ATOM | 3057 | O | LEU | A | 386 | 61.762 | 31.339 | 21.592 | 1.00 | 23.54 | O | |
| ATOM | 3058 | CB | LEU | A | 386 | 62.808 | 33.911 | 23.183 | 1.00 | 20.20 | C | |
| ATOM | 3059 | CG | LEU | A | 386 | 63.483 | 35.298 | 23.058 | 1.00 | 21.58 | C | |
| ATOM | 3060 | CD1 | LEU | A | 386 | 62.642 | 36.337 | 23.771 | 1.00 | 22.42 | C | |
| ATOM | 3061 | CD2 | LEU | A | 386 | 64.926 | 35.276 | 23.654 | 1.00 | 21.55 | C | |
| ATOM | 3062 | N | GLU | A | 387 | 62.601 | 30.772 | 23.617 | 1.00 | 22.74 | N | |
| ATOM | 3063 | CA | GLU | A | 387 | 61.734 | 29.596 | 23.818 | 1.00 | 24.88 | C | |
| ATOM | 3064 | C | GLU | A | 387 | 61.349 | 29.526 | 25.303 | 1.00 | 24.48 | C | |
| ATOM | 3065 | O | GLU | A | 387 | 62.230 | 29.627 | 26.175 | 1.00 | 23.17 | O | |
| ATOM | 3066 | CB | GLU | A | 387 | 62.464 | 28.314 | 23.456 | 1.00 | 27.05 | C | |
| ATOM | 3067 | CG | GLU | A | 387 | 61.503 | 27.128 | 23.237 | 1.00 | 37.16 | C | |
| ATOM | 3068 | CD | GLU | A | 387 | 62.172 | 25.745 | 23.268 | 1.00 | 39.98 | C | |
| ATOM | 3069 | OE1 | GLU | A | 387 | 63.201 | 25.516 | 22.593 | 1.00 | 44.18 | O | |
| ATOM | 3070 | OE2 | GLU | A | 387 | 61.650 | 24.857 | 23.972 | 1.00 | 46.29 | O | |
| ATOM | 3071 | N | LEU | A | 388 | 60.064 | 29.367 | 25.612 | 1.00 | 22.53 | N | |
| ATOM | 3072 | CA | LEU | A | 388 | 59.679 | 29.278 | 27.031 | 1.00 | 26.07 | C | |
| ATOM | 3073 | C | LEU | A | 388 | 59.885 | 27.850 | 27.542 | 1.00 | 27.75 | C | |
| ATOM | 3074 | O | LEU | A | 388 | 59.395 | 26.875 | 26.935 | 1.00 | 28.32 | O | |
| ATOM | 3075 | CB | LEU | A | 388 | 58.224 | 29.686 | 27.281 | 1.00 | 23.47 | C | |
| ATOM | 3076 | CG | LEU | A | 388 | 57.666 | 29.649 | 28.724 | 1.00 | 22.59 | C | |
| ATOM | 3077 | CD1 | LEU | A | 388 | 58.489 | 30.549 | 29.687 | 1.00 | 22.03 | C | |
| ATOM | 3078 | CD2 | LEU | A | 388 | 56.204 | 30.107 | 28.751 | 1.00 | 25.81 | C | |
| ATOM | 3079 | N | LYS | A | 389 | 60.625 | 27.720 | 28.648 | 1.00 | 24.86 | N | |
| ATOM | 3080 | CA | LYS | A | 389 | 60.856 | 26.380 | 29.228 | 1.00 | 27.21 | C | |
| ATOM | 3081 | C | LYS | A | 389 | 59.923 | 26.032 | 30.402 | 1.00 | 26.84 | C | |
| ATOM | 3082 | O | LYS | A | 389 | 59.414 | 24.902 | 30.497 | 1.00 | 27.04 | O | |
| ATOM | 3083 | CB | LYS | A | 389 | 62.310 | 26.246 | 29.735 | 1.00 | 26.39 | C | |
| ATOM | 3084 | CG | LYS | A | 389 | 63.346 | 26.512 | 28.701 | 1.00 | 30.08 | C | |
| ATOM | 3085 | CD | LYS | A | 389 | 63.226 | 25.539 | 27.557 | 1.00 | 33.57 | C | |
| ATOM | 3086 | CE | LYS | A | 389 | 64.360 | 25.699 | 26.571 | 1.00 | 35.29 | C | |
| ATOM | 3087 | NZ | LYS | A | 389 | 65.684 | 25.603 | 27.218 | 1.00 | 36.25 | N | |
| ATOM | 3088 | N | HIS | A | 390 | 59.714 | 26.994 | 31.290 | 1.00 | 25.51 | N | |
| ATOM | 3089 | CA | HIS | A | 390 | 58.893 | 26.802 | 32.476 | 1.00 | 25.54 | C | |
| ATOM | 3090 | C | HIS | A | 390 | 58.355 | 28.139 | 32.990 | 1.00 | 25.92 | C | |
| ATOM | 3091 | O | HIS | A | 390 | 58.976 | 29.182 | 32.776 | 1.00 | 24.09 | O | |
| ATOM | 3092 | CB | HIS | A | 390 | 59.743 | 26.130 | 33.584 | 1.00 | 29.77 | C | |
| ATOM | 3093 | CG | HIS | A | 390 | 58.939 | 25.727 | 34.782 | 1.00 | 33.72 | C | |
| ATOM | 3094 | ND1 | HIS | A | 390 | 58.231 | 24.543 | 34.836 | 1.00 | 35.82 | N | |
| ATOM | 3095 | CD2 | HIS | A | 390 | 58.673 | 26.384 | 35.939 | 1.00 | 32.57 | C | |
| ATOM | 3096 | CE1 | HIS | A | 390 | 57.561 | 24.489 | 35.975 | 1.00 | 37.52 | C | |
| ATOM | 3097 | NE2 | HIS | A | 390 | 57.814 | 25.592 | 36.662 | 1.00 | 39.33 | N | |
| ATOM | 3098 | N | SER | A | 391 | 57.175 | 28.125 | 33.624 | 1.00 | 24.26 | N | |
| ATOM | 3099 | CA | SER | A | 391 | 56.600 | 29.348 | 34.162 | 1.00 | 25.33 | C | |
| ATOM | 3100 | C | SER | A | 391 | 55.911 | 29.073 | 35.490 | 1.00 | 28.33 | C | |
| ATOM | 3101 | O | SER | A | 391 | 55.212 | 28.062 | 35.621 | 1.00 | 28.00 | O | |
| ATOM | 3102 | CB | SER | A | 391 | 55.534 | 29.940 | 33.218 | 1.00 | 24.65 | C | |
| ATOM | 3103 | OG | SER | A | 391 | 54.938 | 31.040 | 33.853 | 1.00 | 25.68 | O | |
| ATOM | 3104 | N | ASN | A | 392 | 56.091 | 29.970 | 36.458 | 1.00 | 26.48 | N | |
| ATOM | 3105 | CA | ASN | A | 392 | 55.396 | 29.833 | 37.731 | 1.00 | 28.76 | C | |
| ATOM | 3106 | C | ASN | A | 392 | 54.343 | 30.930 | 37.878 | 1.00 | 31.72 | C | |
| ATOM | 3107 | O | ASN | A | 392 | 53.783 | 31.109 | 38.964 | 1.00 | 33.11 | O | |
| ATOM | 3108 | CB | ASN | A | 392 | 56.372 | 29.889 | 38.899 | 1.00 | 32.64 | C | |
| ATOM | 3109 | CG | ASN | A | 392 | 57.075 | 28.549 | 39.131 | 1.00 | 35.08 | C | |
| ATOM | 3110 | OD1 | ASN | A | 392 | 56.473 | 27.488 | 38.948 | 1.00 | 37.78 | O | |
| ATOM | 3111 | ND2 | ASN | A | 392 | 58.336 | 28.596 | 39.551 | 1.00 | 36.07 | N | |
| ATOM | 3112 | N | ILE | A | 393 | 54.086 | 31.652 | 36.783 | 1.00 | 30.18 | N | |
| ATOM | 3113 | CA | ILE | A | 393 | 53.110 | 32.742 | 36.735 | 1.00 | 29.96 | C | |
| ATOM | 3114 | C | ILE | A | 393 | 52.044 | 32.460 | 35.642 | 1.00 | 28.49 | C | |
| ATOM | 3115 | O | ILE | A | 393 | 51.394 | 33.395 | 35.155 | 1.00 | 28.83 | O | |
| ATOM | 3116 | CB | ILE | A | 393 | 53.748 | 34.131 | 36.384 | 1.00 | 30.32 | C | |
| ATOM | 3117 | CG1 | ILE | A | 393 | 54.378 | 34.076 | 34.975 | 1.00 | 31.20 | C | |
| ATOM | 3118 | CG2 | ILE | A | 393 | 54.724 | 34.592 | 37.510 | 1.00 | 30.02 | C | |
| ATOM | 3119 | CD1 | ILE | A | 393 | 54.935 | 35.391 | 34.448 | 1.00 | 32.10 | C | |
| ATOM | 3120 | N | GLY | A | 394 | 51.867 | 31.197 | 35.272 | 1.00 | 27.26 | N | |
| ATOM | 3121 | CA | GLY | A | 394 | 50.899 | 30.849 | 34.237 | 1.00 | 31.55 | C | |
| ATOM | 3122 | C | GLY | A | 394 | 51.424 | 30.858 | 32.800 | 1.00 | 34.13 | C | |
| ATOM | 3123 | O | GLY | A | 394 | 52.579 | 31.227 | 32.548 | 1.00 | 31.23 | O | |
| ATOM | 3124 | N | PRO | A | 395 | 50.616 | 30.395 | 31.828 | 1.00 | 36.02 | N | |
| ATOM | 3125 | CA | PRO | A | 395 | 51.095 | 30.399 | 30.437 | 1.00 | 36.00 | C | |
| ATOM | 3126 | C | PRO | A | 395 | 51.157 | 31.835 | 29.918 | 1.00 | 34.59 | C | |
| ATOM | 3127 | O | PRO | A | 395 | 50.467 | 32.707 | 30.435 | 1.00 | 33.77 | O | |
| ATOM | 3128 | CB | PRO | A | 395 | 50.026 | 29.582 | 29.686 | 1.00 | 38.95 | C | |
| ATOM | 3129 | CG | PRO | A | 395 | 49.471 | 28.674 | 30.737 | 1.00 | 39.78 | C | |
| ATOM | 3130 | CD | PRO | A | 395 | 49.387 | 29.589 | 31.957 | 1.00 | 36.96 | C | |
| ATOM | 3131 | N | PHE | A | 396 | 52.033 | 32.074 | 28.942 | 1.00 | 32.83 | N | |
| ATOM | 3132 | CA | PHE | A | 396 | 52.151 | 33.376 | 28.281 | 1.00 | 30.03 | C | |
| ATOM | 3133 | C | PHE | A | 396 | 53.033 | 33.166 | 27.064 | 1.00 | 30.18 | C | |
| ATOM | 3134 | O | PHE | A | 396 | 53.814 | 32.216 | 27.003 | 1.00 | 30.26 | O | |
| ATOM | 3135 | CB | PHE | A | 396 | 52.695 | 34.477 | 29.219 | 1.00 | 28.59 | C | |
| ATOM | 3136 | CG | PHE | A | 396 | 54.133 | 34.292 | 29.643 | 1.00 | 28.56 | C | |
| ATOM | 3137 | CD1 | PHE | A | 396 | 55.181 | 34.788 | 28.858 | 1.00 | 29.91 | C | |
| ATOM | 3138 | CD2 | PHE | A | 396 | 54.434 | 33.638 | 30.837 | 1.00 | 28.62 | C | |
| ATOM | 3139 | CE1 | PHE | A | 396 | 56.553 | 34.636 | 29.260 | 1.00 | 28.42 | C | |
| ATOM | 3140 | CE2 | PHE | A | 396 | 55.776 | 33.477 | 31.257 | 1.00 | 29.06 | C | |
| ATOM | 3141 | CZ | PHE | A | 396 | 56.843 | 33.982 | 30.450 | 1.00 | 25.50 | C | |
| ATOM | 3142 | N | PRO | A | 397 | 52.882 | 34.012 | 26.039 | 1.00 | 31.72 | N | |
| ATOM | 3143 | CA | PRO | A | 397 | 53.695 | 33.881 | 24.811 | 1.00 | 30.99 | C | |
| ATOM | 3144 | C | PRO | A | 397 | 55.089 | 34.437 | 25.073 | 1.00 | 25.03 | C | |
| ATOM | 3145 | O | PRO | A | 397 | 55.209 | 35.577 | 25.501 | 1.00 | 25.74 | O | |
| ATOM | 3146 | CB | PRO | A | 397 | 52.949 | 34.744 | 23.782 | 1.00 | 33.15 | C | |
| ATOM | 3147 | CG | PRO | A | 397 | 51.586 | 34.999 | 24.401 | 1.00 | 36.83 | C | |
| ATOM | 3148 | CD | PRO | A | 397 | 51.847 | 35.046 | 25.893 | 1.00 | 33.37 | C | |
| ATOM | 3149 | N | VAL | A | 398 | 56.108 | 33.641 | 24.798 | 1.00 | 28.32 | N | |
| ATOM | 3150 | CA | VAL | A | 398 | 57.498 | 34.072 | 25.049 | 1.00 | 27.81 | C | |
| ATOM | 3151 | C | VAL | A | 398 | 57.856 | 35.395 | 24.331 | 1.00 | 27.85 | C | |
| ATOM | 3152 | O | VAL | A | 398 | 58.757 | 36.123 | 24.746 | 1.00 | 24.15 | O | |
| ATOM | 3153 | CB | VAL | A | 398 | 58.474 | 32.888 | 24.702 | 1.00 | 26.70 | C | |
| ATOM | 3154 | CG1 | VAL | A | 398 | 58.766 | 32.848 | 23.215 | 1.00 | 27.76 | C | |
| ATOM | 3155 | CG2 | VAL | A | 398 | 59.774 | 32.999 | 25.558 | 1.00 | 25.31 | C | |
| ATOM | 3156 | N | GLU | A | 399 | 57.102 | 35.742 | 23.280 | 1.00 | 26.71 | N | |
| ATOM | 3157 | CA | GLU | A | 399 | 57.345 | 36.985 | 22.548 | 1.00 | 28.24 | C | |
| ATOM | 3158 | C | GLU | A | 399 | 57.228 | 38.237 | 23.401 | 1.00 | 26.94 | C | |
| ATOM | 3159 | O | GLU | A | 399 | 57.781 | 39.288 | 23.047 | 1.00 | 30.28 | O | |
| ATOM | 3160 | CB | GLU | A | 399 | 56.365 | 37.122 | 21.367 | 1.00 | 29.28 | C | |
| ATOM | 3161 | CG | GLU | A | 399 | 56.616 | 36.134 | 20.251 | 1.00 | 33.82 | C | |
| ATOM | 3162 | CD | GLU | A | 399 | 56.400 | 34.677 | 20.666 | 1.00 | 36.50 | C | |
| ATOM | 3163 | OE1 | GLU | A | 399 | 55.470 | 34.413 | 21.483 | 1.00 | 30.49 | O | |
| ATOM | 3164 | OE2 | GLU | A | 399 | 57.174 | 33.811 | 20.154 | 1.00 | 37.52 | O | |
| ATOM | 3165 | N | LEU | A | 400 | 56.493 | 38.157 | 24.505 | 1.00 | 27.36 | N | |
| ATOM | 3166 | CA | LEU | A | 400 | 56.364 | 39.312 | 25.363 | 1.00 | 29.08 | C | |
| ATOM | 3167 | C | LEU | A | 400 | 57.719 | 39.681 | 25.963 | 1.00 | 27.76 | C | |
| ATOM | 3168 | O | LEU | A | 400 | 57.899 | 40.793 | 26.382 | 1.00 | 29.05 | O | |
| ATOM | 3169 | CB | LEU | A | 400 | 55.362 | 39.053 | 26.496 | 1.00 | 29.59 | C | |
| ATOM | 3170 | CG | LEU | A | 400 | 53.940 | 38.807 | 26.046 | 1.00 | 32.52 | C | |
| ATOM | 3171 | CD1 | LEU | A | 400 | 53.027 | 38.505 | 27.241 | 1.00 | 31.14 | C | |
| ATOM | 3172 | CD2 | LEU | A | 400 | 53.484 | 40.056 | 25.314 | 1.00 | 36.13 | C | |
| ATOM | 3173 | N | LEU | A | 401 | 58.668 | 38.748 | 25.993 | 1.00 | 27.77 | N | |
| ATOM | 3174 | CA | LEU | A | 401 | 59.992 | 39.047 | 26.559 | 1.00 | 25.80 | C | |
| ATOM | 3175 | C | LEU | A | 401 | 61.014 | 39.497 | 25.526 | 1.00 | 26.73 | C | |
| ATOM | 3176 | O | LEU | A | 401 | 62.151 | 39.842 | 25.872 | 1.00 | 27.57 | O | |
| ATOM | 3177 | CB | LEU | A | 401 | 60.544 | 37.815 | 27.289 | 1.00 | 22.41 | C | |
| ATOM | 3178 | CG | LEU | A | 401 | 59.721 | 37.451 | 28.526 | 1.00 | 23.32 | C | |
| ATOM | 3179 | CD1 | LEU | A | 401 | 60.133 | 36.055 | 28.969 | 1.00 | 26.06 | C | |
| ATOM | 3180 | CD2 | LEU | A | 401 | 59.921 | 38.497 | 29.649 | 1.00 | 22.32 | C | |
| ATOM | 3181 | N | GLN | A | 402 | 60.618 | 39.510 | 24.253 | 1.00 | 25.66 | N | |
| ATOM | 3182 | CA | GLN | A | 402 | 61.554 | 39.908 | 23.207 | 1.00 | 25.39 | C | |
| ATOM | 3183 | C | GLN | A | 402 | 62.178 | 41.311 | 23.400 | 1.00 | 23.36 | C | |
| ATOM | 3184 | O | GLN | A | 402 | 63.403 | 41.455 | 23.306 | 1.00 | 23.87 | O | |
| ATOM | 3185 | CB | GLN | A | 402 | 60.884 | 39.811 | 21.819 | 1.00 | 24.98 | C | |
| ATOM | 3186 | CG | GLN | A | 402 | 61.896 | 39.857 | 20.675 | 1.00 | 28.31 | C | |
| ATOM | 3187 | CD | GLN | A | 402 | 62.645 | 38.536 | 20.492 | 1.00 | 28.84 | C | |
| ATOM | 3188 | OE1 | GLN | A | 402 | 62.034 | 37.477 | 20.266 | 1.00 | 28.57 | O | |
| ATOM | 3189 | NE2 | GLN | A | 402 | 63.957 | 38.595 | 20.579 | 1.00 | 23.99 | N | |
| ATOM | 3190 | N | ASP | A | 403 | 61.379 | 42.340 | 23.700 | 1.00 | 22.96 | N | |
| ATOM | 3191 | CA | ASP | A | 403 | 61.970 | 43.664 | 23.857 | 1.00 | 24.09 | C | |
| ATOM | 3192 | C | ASP | A | 403 | 63.005 | 43.800 | 24.983 | 1.00 | 25.21 | C | |
| ATOM | 3193 | O | ASP | A | 403 | 64.048 | 44.438 | 24.807 | 1.00 | 24.96 | O | |
| ATOM | 3194 | CB | ASP | A | 403 | 60.884 | 44.734 | 24.080 | 1.00 | 27.07 | C | |
| ATOM | 3195 | CG | ASP | A | 403 | 59.962 | 44.877 | 22.883 | 1.00 | 31.43 | C | |
| ATOM | 3196 | OD1 | ASP | A | 403 | 60.391 | 44.477 | 21.768 | 1.00 | 28.23 | O | |
| ATOM | 3197 | OD2 | ASP | A | 403 | 58.833 | 45.403 | 23.077 | 1.00 | 32.56 | O | |
| ATOM | 3198 | N | ILE | A | 404 | 62.707 | 43.231 | 26.147 | 1.00 | 23.69 | N | |
| ATOM | 3199 | CA | ILE | A | 404 | 63.641 | 43.385 | 27.244 | 1.00 | 22.82 | C | |
| ATOM | 3200 | C | ILE | A | 404 | 64.902 | 42.587 | 26.923 | 1.00 | 23.27 | C | |
| ATOM | 3201 | O | ILE | A | 404 | 65.993 | 43.048 | 27.218 | 1.00 | 22.20 | O | |
| ATOM | 3202 | CB | ILE | A | 404 | 62.988 | 42.956 | 28.571 | 1.00 | 24.00 | C | |
| ATOM | 3203 | CG1 | ILE | A | 404 | 63.958 | 43.222 | 29.723 | 1.00 | 27.72 | C | |
| ATOM | 3204 | CG2 | ILE | A | 404 | 62.516 | 41.533 | 28.472 | 1.00 | 23.88 | C | |
| ATOM | 3205 | CD1 | ILE | A | 404 | 63.268 | 43.632 | 31.034 | 1.00 | 31.59 | C | |
| ATOM | 3206 | N | MET | A | 405 | 64.762 | 41.428 | 26.273 | 1.00 | 22.62 | N | |
| ATOM | 3207 | CA | MET | A | 405 | 65.945 | 40.635 | 25.940 | 1.00 | 23.69 | C | |
| ATOM | 3208 | C | MET | A | 405 | 66.774 | 41.330 | 24.842 | 1.00 | 23.83 | C | |
| ATOM | 3209 | O | MET | A | 405 | 68.008 | 41.322 | 24.887 | 1.00 | 21.48 | O | |
| ATOM | 3210 | CB | MET | A | 405 | 65.558 | 39.189 | 25.541 | 1.00 | 24.44 | C | |
| ATOM | 3211 | CG | MET | A | 405 | 64.834 | 38.424 | 26.658 | 1.00 | 26.47 | C | |
| ATOM | 3212 | SD | MET | A | 405 | 65.782 | 38.427 | 28.249 | 1.00 | 31.28 | S | |
| ATOM | 3213 | CE | MET | A | 405 | 66.985 | 37.529 | 27.697 | 1.00 | 27.02 | C | |
| ATOM | 3214 | N | ASN | A | 406 | 66.108 | 41.997 | 23.892 | 1.00 | 25.32 | N | |
| ATOM | 3215 | CA | ASN | A | 406 | 66.821 | 42.700 | 22.803 | 1.00 | 25.01 | C | |
| ATOM | 3216 | C | ASN | A | 406 | 67.659 | 43.868 | 23.339 | 1.00 | 25.74 | C | |
| ATOM | 3217 | O | ASN | A | 406 | 68.622 | 44.306 | 22.685 | 1.00 | 23.75 | O | |
| ATOM | 3218 | CB | ASN | A | 406 | 65.829 | 43.198 | 21.712 | 1.00 | 24.29 | C | |
| ATOM | 3219 | CG | ASN | A | 406 | 65.341 | 42.062 | 20.789 | 1.00 | 24.00 | C | |
| ATOM | 3220 | OD1 | ASN | A | 406 | 65.830 | 40.942 | 20.857 | 1.00 | 28.11 | O | |
| ATOM | 3221 | ND2 | ASN | A | 406 | 64.363 | 42.367 | 19.906 | 1.00 | 24.97 | N | |
| ATOM | 3222 | N | TYR | A | 407 | 67.299 | 44.356 | 24.532 | 1.00 | 23.43 | N | |
| ATOM | 3223 | CA | TYR | A | 407 | 68.029 | 49.434 | 25.166 | 1.00 | 24.18 | C | |
| ATOM | 3224 | C | TYR | A | 407 | 69.144 | 44.861 | 26.052 | 1.00 | 22.82 | C | |
| ATOM | 3225 | O | TYR | A | 407 | 70.354 | 45.119 | 25.829 | 1.00 | 21.93 | O | |
| ATOM | 3226 | CB | TYR | A | 407 | 67.066 | 46.274 | 26.010 | 1.00 | 27.92 | C | |
| ATOM | 3227 | CG | TYR | A | 407 | 67.751 | 47.430 | 26.690 | 1.00 | 33.37 | C | |
| ATOM | 3228 | CD1 | TYR | A | 407 | 68.167 | 48.545 | 25.955 | 1.00 | 34.55 | C | |
| ATOM | 3229 | CD2 | TYR | A | 407 | 68.058 | 47.380 | 28.049 | 1.00 | 33.57 | C | |
| ATOM | 3230 | CE1 | TYR | A | 407 | 68.876 | 49.574 | 26.558 | 1.00 | 38.53 | C | |
| ATOM | 3231 | CE2 | TYR | A | 407 | 68.761 | 48.402 | 28.656 | 1.00 | 38.93 | C | |
| ATOM | 3232 | CZ | TYR | A | 407 | 69.170 | 49.492 | 27.914 | 1.00 | 38.57 | C | |
| ATOM | 3233 | OH | TYR | A | 407 | 69.879 | 50.504 | 28.529 | 1.00 | 41.58 | O | |
| ATOM | 3234 | N | ILE | A | 408 | 68.738 | 44.065 | 27.034 | 1.00 | 22.57 | N | |
| ATOM | 3235 | CA | ILE | A | 408 | 69.681 | 43.439 | 27.981 | 1.00 | 24.17 | C | |
| ATOM | 3236 | C | ILE | A | 408 | 70.892 | 42.753 | 27.338 | 1.00 | 23.46 | C | |
| ATOM | 3237 | O | ILE | A | 408 | 72.002 | 42.873 | 27.825 | 1.00 | 24.38 | O | |
| ATOM | 3238 | CB | ILE | A | 408 | 68.936 | 42.427 | 28.887 | 1.00 | 27.96 | C | |
| ATOM | 3239 | CG1 | ILE | A | 408 | 68.214 | 43.204 | 29.965 | 1.00 | 31.20 | C | |
| ATOM | 3240 | CG2 | ILE | A | 408 | 69.900 | 41.388 | 29.498 | 1.00 | 32.51 | C | |
| ATOM | 3241 | CD1 | ILE | A | 408 | 67.235 | 42.347 | 30.717 | 1.00 | 34.27 | C | |
| ATOM | 3242 | N | VAL | A | 409 | 70.695 | 42.017 | 26.250 | 1.00 | 24.76 | N | |
| ATOM | 3243 | CA | VAL | A | 409 | 71.828 | 41.335 | 25.645 | 1.00 | 23.94 | C | |
| ATOM | 3244 | C | VAL | A | 409 | 72.953 | 42.192 | 25.083 | 1.00 | 26.77 | C | |
| ATOM | 3245 | O | VAL | A | 409 | 74.074 | 42.096 | 25.554 | 1.00 | 25.67 | O | |
| ATOM | 3246 | CB | VAL | A | 409 | 71.317 | 40.353 | 24.611 | 1.00 | 30.46 | C | |
| ATOM | 3247 | CG1 | VAL | A | 409 | 72.448 | 39.951 | 23.658 | 1.00 | 28.71 | C | |
| ATOM | 3248 | CG2 | VAL | A | 409 | 70.672 | 39.175 | 25.347 | 1.00 | 24.93 | C | |
| ATOM | 3249 | N | PRO | A | 410 | 72.686 | 43.085 | 24.091 | 1.00 | 26.72 | N | |
| ATOM | 3250 | CA | PRO | A | 410 | 73.799 | 43.893 | 23.565 | 1.00 | 26.51 | C | |
| ATOM | 3251 | C | PRO | A | 410 | 74.300 | 44.993 | 24.515 | 1.00 | 25.72 | C | |
| ATOM | 3252 | O | PRO | A | 410 | 75.468 | 45.438 | 24.451 | 1.00 | 26.09 | O | |
| ATOM | 3253 | CB | PRO | A | 410 | 73.213 | 44.470 | 22.252 | 1.00 | 29.07 | C | |
| ATOM | 3254 | CG | PRO | A | 410 | 71.741 | 44.611 | 22.576 | 1.00 | 28.83 | C | |
| ATOM | 3255 | CD | PRO | A | 410 | 71.450 | 43.308 | 23.319 | 1.00 | 27.31 | C | |
| ATOM | 3256 | N | ILE | A | 411 | 73.426 | 45.447 | 25.402 | 1.00 | 22.55 | N | |
| ATOM | 3257 | CA | ILE | A | 411 | 73.842 | 46.484 | 26.333 | 1.00 | 25.31 | C | |
| ATOM | 3258 | C | ILE | A | 411 | 74.587 | 45.988 | 27.585 | 1.00 | 25.38 | C | |
| ATOM | 3259 | O | ILE | A | 411 | 75.588 | 46.575 | 27.971 | 1.00 | 25.04 | O | |
| ATOM | 3260 | CB | ILE | A | 411 | 72.634 | 47.294 | 26.833 | 1.00 | 30.13 | C | |
| ATOM | 3261 | CG1 | ILE | A | 411 | 71.970 | 48.007 | 25.651 | 1.00 | 33.77 | C | |
| ATOM | 3262 | CG2 | ILE | A | 411 | 73.072 | 48.291 | 27.874 | 1.00 | 31.49 | C | |
| ATOM | 3263 | CD1 | ILE | A | 411 | 72.804 | 49.140 | 25.103 | 1.00 | 38.80 | C | |
| ATOM | 3264 | N | LEU | A | 412 | 74.102 | 44.900 | 28.192 | 1.00 | 23.47 | N | |
| ATOM | 3265 | CA | LEU | A | 412 | 74.698 | 44.420 | 29.431 | 1.00 | 24.03 | C | |
| ATOM | 3266 | C | LEU | A | 412 | 75.429 | 43.082 | 29.373 | 1.00 | 24.12 | C | |
| ATOM | 3267 | O | LEU | A | 412 | 76.404 | 42.906 | 30.089 | 1.00 | 24.47 | O | |
| ATOM | 3268 | CB | LEU | A | 412 | 73.638 | 44.311 | 30.512 | 1.00 | 26.07 | C | |
| ATOM | 3269 | CG | LEU | A | 412 | 72.904 | 45.582 | 30.946 | 1.00 | 31.04 | C | |
| ATOM | 3270 | CD1 | LEU | A | 412 | 71.764 | 45.236 | 31.883 | 1.00 | 28.70 | C | |
| ATOM | 3271 | CD2 | LEU | A | 412 | 73.931 | 46.493 | 31.681 | 1.00 | 29.45 | C | |
| ATOM | 3272 | N | VAL | A | 413 | 74.966 | 42.143 | 28.550 | 1.00 | 19.71 | N | |
| ATOM | 3273 | CA | VAL | A | 413 | 75.630 | 40.815 | 28.508 | 1.00 | 20.71 | C | |
| ATOM | 3274 | C | VAL | A | 413 | 76.776 | 40.677 | 27.515 | 1.00 | 19.12 | C | |
| ATOM | 3275 | O | VAL | A | 413 | 77.899 | 40.309 | 27.907 | 1.00 | 20.36 | O | |
| ATOM | 3276 | CB | VAL | A | 413 | 74.588 | 39.665 | 28.247 | 1.00 | 23.74 | C | |
| ATOM | 3277 | CG1 | VAL | A | 413 | 75.277 | 38.304 | 28.398 | 1.00 | 22.41 | C | |
| ATOM | 3278 | CG2 | VAL | A | 413 | 73.408 | 39.797 | 29.204 | 1.00 | 25.05 | C | |
| ATOM | 3279 | N | LEU | A | 414 | 76.521 | 40.964 | 26.234 | 1.00 | 16.47 | N | |
| ATOM | 3280 | CA | LEU | A | 414 | 77.551 | 40.904 | 25.217 | 1.00 | 17.77 | C | |
| ATOM | 3281 | C | LEU | A | 414 | 78.846 | 41.686 | 25.529 | 1.00 | 17.11 | C | |
| ATOM | 3282 | O | LEU | A | 414 | 79.938 | 41.196 | 25.226 | 1.00 | 19.03 | O | |
| ATOM | 3283 | CB | LEU | A | 414 | 77.015 | 41.345 | 23.832 | 1.00 | 18.58 | C | |
| ATOM | 3284 | CG | LEU | A | 414 | 77.989 | 41.222 | 22.646 | 1.00 | 19.25 | C | |
| ATOM | 3285 | CD1 | LEU | A | 414 | 78.439 | 39.725 | 22.442 | 1.00 | 24.16 | C | |
| ATOM | 3286 | CD2 | LEU | A | 414 | 77.270 | 41.708 | 21.397 | 1.00 | 23.16 | C | |
| ATOM | 3287 | N | PRO | A | 415 | 78.752 | 42.895 | 26.089 | 1.00 | 19.05 | N | |
| ATOM | 3288 | CA | PRO | A | 415 | 79.993 | 43.620 | 26.370 | 1.00 | 19.48 | C | |
| ATOM | 3289 | C | PRO | A | 415 | 80.941 | 42.886 | 27.304 | 1.00 | 21.66 | C | |
| ATOM | 3290 | O | PRO | A | 415 | 82.165 | 42.926 | 27.109 | 1.00 | 19.36 | O | |
| ATOM | 3291 | CB | PRO | A | 415 | 79.494 | 44.939 | 26.997 | 1.00 | 19.33 | C | |
| ATOM | 3292 | CG | PRO | A | 415 | 78.226 | 45.179 | 26.302 | 1.00 | 24.09 | C | |
| ATOM | 3293 | CD | PRO | A | 415 | 77.583 | 43.771 | 26.331 | 1.00 | 19.42 | C | |
| ATOM | 3294 | N | ARG | A | 416 | 80.389 | 42.205 | 28.304 | 1.00 | 19.80 | N | |
| ATOM | 3295 | CA | ARG | A | 416 | 81.270 | 41.466 | 29.231 | 1.00 | 21.10 | C | |
| ATOM | 3296 | C | ARG | A | 416 | 82.018 | 40.387 | 28.463 | 1.00 | 19.17 | C | |
| ATOM | 3297 | O | ARG | A | 416 | 83.172 | 40.116 | 28.717 | 1.00 | 19.00 | O | |
| ATOM | 3298 | CB | ARG | A | 416 | 80.448 | 40.837 | 30.351 | 1.00 | 23.65 | C | |
| ATOM | 3299 | CG | ARG | A | 416 | 79.581 | 41.852 | 31.095 | 1.00 | 27.82 | C | |
| ATOM | 3300 | CD | ARG | A | 416 | 80.435 | 42.709 | 32.023 | 1.00 | 33.30 | C | |
| ATOM | 3301 | NE | ARG | A | 416 | 80.994 | 43.881 | 31.353 | 1.00 | 39.79 | N | |
| ATOM | 3302 | CZ | ARG | A | 416 | 80.259 | 44.877 | 30.837 | 1.00 | 42.30 | C | |
| ATOM | 3303 | NH1 | ARG | A | 416 | 78.914 | 44.839 | 30.908 | 1.00 | 40.30 | N | |
| ATOM | 3304 | NH2 | ARG | A | 416 | 80.874 | 45.916 | 30.253 | 1.00 | 39.73 | N | |
| ATOM | 3305 | N | VAL | A | 417 | 81.348 | 39.791 | 27.495 | 1.00 | 19.02 | N | |
| ATOM | 3306 | CA | VAL | A | 417 | 81.962 | 38.768 | 26.652 | 1.00 | 22.54 | C | |
| ATOM | 3307 | C | VAL | A | 417 | 83.081 | 39.351 | 25.777 | 1.00 | 16.77 | C | |
| ATOM | 3308 | O | VAL | A | 417 | 84.194 | 38.828 | 25.715 | 1.00 | 16.49 | O | |
| ATOM | 3309 | CB | VAL | A | 417 | 80.879 | 38.145 | 25.779 | 1.00 | 22.64 | C | |
| ATOM | 3310 | CG1 | VAL | A | 417 | 81.407 | 37.779 | 24.438 | 1.00 | 36.94 | C | |
| ATOM | 3311 | CG2 | VAL | A | 417 | 80.338 | 36.939 | 26.478 | 1.00 | 36.93 | C | |
| ATOM | 3312 | N | ASN | A | 418 | 82.770 | 40.440 | 25.075 | 1.00 | 15.55 | N | |
| ATOM | 3313 | CA | ASN | A | 418 | 83.722 | 41.065 | 24.231 | 1.00 | 14.24 | C | |
| ATOM | 3314 | C | ASN | A | 418 | 84.917 | 41.675 | 24.949 | 1.00 | 14.58 | C | |
| ATOM | 3315 | O | ASN | A | 418 | 85.968 | 41.782 | 24.318 | 1.00 | 14.77 | O | |
| ATOM | 3316 | CB | ASN | A | 418 | 82.997 | 42.087 | 23.305 | 1.00 | 16.36 | C | |
| ATOM | 3317 | CG | ASN | A | 418 | 82.402 | 41.404 | 22.061 | 1.00 | 19.97 | C | |
| ATOM | 3318 | OD1 | ASN | A | 418 | 82.980 | 40.422 | 21.553 | 1.00 | 18.89 | O | |
| ATOM | 3319 | ND2 | ASN | A | 418 | 81.300 | 41.920 | 21.553 | 1.00 | 17.82 | N | |
| ATOM | 3320 | N | GLU | A | 419 | 84.769 | 42.093 | 26.217 | 1.00 | 14.50 | N | |
| ATOM | 3321 | CA | GLU | A | 419 | 85.928 | 42.590 | 26.986 | 1.00 | 19.31 | C | |
| ATOM | 3322 | C | GLU | A | 419 | 86.934 | 41.443 | 27.153 | 1.00 | 16.18 | C | |
| ATOM | 3323 | O | GLU | A | 419 | 88.111 | 41.627 | 27.010 | 1.00 | 18.20 | O | |
| ATOM | 3324 | CB | GLU | A | 419 | 85.501 | 43.085 | 28.354 | 1.00 | 22.44 | C | |
| ATOM | 3325 | CG | GLU | A | 419 | 84.868 | 44.434 | 28.299 | 1.00 | 29.68 | C | |
| ATOM | 3326 | CD | GLU | A | 419 | 84.234 | 44.746 | 29.629 | 1.00 | 32.95 | C | |
| ATOM | 3327 | OE1 | GLU | A | 419 | 84.859 | 44.423 | 30.652 | 1.00 | 41.89 | O | |
| ATOM | 3328 | OE2 | GLU | A | 419 | 83.120 | 45.298 | 29.634 | 1.00 | 42.80 | O | |
| ATOM | 3329 | N | LYS | A | 420 | 86.423 | 40.246 | 27.425 | 1.00 | 18.41 | N | |
| ATOM | 3330 | CA | LYS | A | 420 | 87.305 | 39.096 | 27.568 | 1.00 | 19.49 | C | |
| ATOM | 3331 | C | LYS | A | 420 | 87.917 | 38.617 | 26.233 | 1.00 | 19.26 | C | |
| ATOM | 3332 | O | LYS | A | 420 | 89.085 | 38.244 | 26.180 | 1.00 | 19.03 | O | |
| ATOM | 3333 | CB | LYS | A | 420 | 86.559 | 37.959 | 28.272 | 1.00 | 24.86 | C | |
| ATOM | 3334 | CG | LYS | A | 420 | 87.485 | 36.748 | 28.441 | 1.00 | 26.08 | C | |
| ATOM | 3335 | CD | LYS | A | 420 | 88.576 | 36.974 | 29.526 | 1.00 | 28.43 | C | |
| ATOM | 3336 | CE | LYS | A | 420 | 89.518 | 35.743 | 29.639 | 1.00 | 33.40 | C | |
| ATOM | 3337 | NZ | LYS | A | 420 | 90.694 | 35.904 | 30.610 | 1.00 | 23.76 | N | |
| ATOM | 3338 | N | LEU | A | 421 | 87.126 | 38.654 | 25.139 | 1.00 | 19.68 | N | |
| ATOM | 3339 | CA | LEU | A | 421 | 87.625 | 38.253 | 23.826 | 1.00 | 21.74 | C | |
| ATOM | 3340 | C | LEU | A | 421 | 88.700 | 39.238 | 23.338 | 1.00 | 18.03 | C | |
| ATOM | 3341 | O | LEU | A | 421 | 89.656 | 38.864 | 22.661 | 1.00 | 18.45 | O | |
| ATOM | 3342 | CB | LEU | A | 421 | 86.442 | 38.185 | 22.850 | 1.00 | 20.55 | C | |
| ATOM | 3343 | CG | LEU | A | 421 | 85.483 | 37.060 | 23.215 | 1.00 | 21.71 | C | |
| ATOM | 3344 | CD1 | LEU | A | 421 | 84.179 | 37.245 | 22.433 | 1.00 | 24.60 | C | |
| ATOM | 3345 | CD2 | LEU | A | 421 | 86.141 | 35.719 | 22.866 | 1.00 | 22.63 | C | |
| ATOM | 3346 | N | GLN | A | 422 | 88.550 | 40.508 | 23.692 | 1.00 | 18.48 | N | |
| ATOM | 3347 | CA | GLN | A | 422 | 89.557 | 41.499 | 23.327 | 1.00 | 19.41 | C | |
| ATOM | 3348 | C | GLN | A | 422 | 90.847 | 41.397 | 24.127 | 1.00 | 18.53 | C | |
| ATOM | 3349 | O | GLN | A | 422 | 91.907 | 41.756 | 23.603 | 1.00 | 22.07 | O | |
| ATOM | 3350 | CB | GLN | A | 422 | 88.978 | 42.938 | 23.421 | 1.00 | 20.17 | C | |
| ATOM | 3351 | CG | GLN | A | 422 | 88.115 | 43.246 | 22.179 | 1.00 | 22.16 | C | |
| ATOM | 3352 | CD | GLN | A | 422 | 88.979 | 43.478 | 20.908 | 1.00 | 19.83 | C | |
| ATOM | 3353 | OE1 | GLN | A | 422 | 89.840 | 44.402 | 20.861 | 1.00 | 26.58 | O | |
| ATOM | 3354 | NE2 | GLN | A | 422 | 88.763 | 42.654 | 19.887 | 1.00 | 23.03 | N | |
| ATOM | 3355 | N | LYS | A | 423 | 90.767 | 40.909 | 25.369 | 1.00 | 19.30 | N | |
| ATOM | 3356 | CA | LYS | A | 423 | 91.945 | 40.710 | 26.211 | 1.00 | 21.18 | C | |
| ATOM | 3357 | C | LYS | A | 423 | 92.758 | 39.605 | 25.502 | 1.00 | 23.74 | C | |
| ATOM | 3358 | O | LYS | A | 423 | 93.983 | 39.598 | 25.520 | 1.00 | 24.05 | O | |
| ATOM | 3359 | CB | LYS | A | 423 | 91.519 | 40.264 | 27.619 | 1.00 | 22.13 | C | |
| ATOM | 3360 | CG | LYS | A | 423 | 92.683 | 39.870 | 28.501 | 1.00 | 28.49 | C | |
| ATOM | 3361 | CD | LYS | A | 423 | 92.239 | 39.223 | 29.842 | 1.00 | 34.87 | C | |
| ATOM | 3362 | CE | LYS | A | 423 | 93.422 | 38.373 | 30.382 | 1.00 | 36.10 | C | |
| ATOM | 3363 | NZ | LYS | A | 423 | 93.195 | 37.702 | 31.680 | 1.00 | 41.82 | N | |
| ATOM | 3364 | N | GLY | A | 424 | 92.040 | 38.684 | 24.873 | 1.00 | 22.38 | N | |
| ATOM | 3365 | CA | GLY | A | 424 | 92.679 | 37.641 | 24.079 | 1.00 | 22.35 | C | |
| ATOM | 3366 | C | GLY | A | 424 | 93.355 | 36.440 | 24.745 | 1.00 | 19.62 | C | |
| ATOM | 3367 | O | GLY | A | 424 | 93.200 | 36.206 | 25.947 | 1.00 | 22.15 | O | |
| ATOM | 3368 | N | APHE | A | 425 | 94.119 | 35.697 | 23.948 | 0.50 | 18.88 | N | |
| ATOM | 3369 | N | BPHE | A | 425 | 94.107 | 35.708 | 23.923 | 0.50 | 20.46 | N | |
| ATOM | 3370 | CA | APHE | A | 425 | 94.822 | 34.497 | 24.434 | 0.50 | 16.32 | C | |
| ATOM | 3371 | CA | BPHE | A | 425 | 94.790 | 34.473 | 24.332 | 0.50 | 19.97 | C | |
| ATOM | 3372 | C | APHE | A | 425 | 96.190 | 34.458 | 23.787 | 0.50 | 15.57 | C | |
| ATOM | 3373 | C | BPHE | A | 425 | 96.201 | 34.439 | 23.746 | 0.50 | 17.46 | C | |
| ATOM | 3374 | O | APHE | A | 425 | 96.362 | 34.816 | 22.614 | 0.50 | 18.72 | O | |
| ATOM | 3375 | O | BPHE | A | 425 | 96.414 | 34.802 | 22.579 | 0.50 | 19.98 | O | |
| ATOM | 3376 | CB | APHE | A | 425 | 93.981 | 33.244 | 24.101 | 0.50 | 15.89 | C | |
| ATOM | 3377 | CB | BPHE | A | 425 | 93.980 | 33.247 | 23.810 | 0.50 | 22.42 | C | |
| ATOM | 3378 | CG | APHE | A | 425 | 92.583 | 33.332 | 24.650 | 0.50 | 19.13 | C | |
| ATOM | 3379 | CG | BPHE | A | 425 | 92.489 | 33.335 | 24.088 | 0.50 | 30.09 | C | |
| ATOM | 3380 | CD1 | APHE | A | 425 | 92.327 | 33.048 | 25.988 | 0.50 | 16.03 | C | |
| ATOM | 3381 | CD1 | BPHE | A | 425 | 91.746 | 34.453 | 23.703 | 0.50 | 31.10 | C | |
| ATOM | 3382 | CD2 | APHE | A | 425 | 91.545 | 33.818 | 23.865 | 0.50 | 20.90 | C | |
| ATOM | 3383 | CD2 | BPHE | A | 425 | 91.847 | 32.345 | 24.811 | 0.50 | 33.54 | C | |
| ATOM | 3384 | CE1 | APHE | A | 425 | 91.051 | 33.246 | 26.551 | 0.50 | 16.38 | C | |
| ATOM | 3385 | CE1 | BPHE | A | 425 | 90.387 | 34.587 | 24.054 | 0.50 | 33.46 | C | |
| ATOM | 3386 | CE2 | APHE | A | 425 | 90.259 | 34.025 | 24.429 | 0.50 | 21.73 | C | |
| ATOM | 3387 | CE2 | BPHE | A | 425 | 90.491 | 32.459 | 25.165 | 0.50 | 35.61 | C | |
| ATOM | 3388 | CZ | APHE | A | 425 | 90.033 | 33.732 | 25.773 | 0.50 | 15.74 | C | |
| ATOM | 3389 | CZ | BPHE | A | 425 | 89.763 | 33.583 | 24.791 | 0.50 | 35.59 | C | |
| ATOM | 3390 | N | PRO | A | 426 | 97.194 | 34.013 | 24.543 | 1.00 | 15.33 | N | |
| ATOM | 3391 | CA | PRO | A | 426 | 98.587 | 33.935 | 24.058 | 1.00 | 17.85 | C | |
| ATOM | 3392 | C | PRO | A | 426 | 98.749 | 32.890 | 22.944 | 1.00 | 19.06 | C | |
| ATOM | 3393 | O | PRO | A | 426 | 98.135 | 31.825 | 23.021 | 1.00 | 18.81 | O | |
| ATOM | 3394 | CB | PRO | A | 426 | 99.394 | 33.492 | 25.317 | 1.00 | 20.75 | C | |
| ATOM | 3395 | CG | PRO | A | 426 | 98.415 | 33.397 | 26.418 | 1.00 | 18.10 | C | |
| ATOM | 3396 | CD | PRO | A | 426 | 97.022 | 33.367 | 25.843 | 1.00 | 16.62 | C | |
| ATOM | 3397 | N | LEU | A | 427 | 99.556 | 33.251 | 21.932 | 1.00 | 17.88 | N | |
| ATOM | 3398 | CA | LEU | A | 427 | 99.940 | 32.379 | 20.803 | 1.00 | 19.44 | C | |
| ATOM | 3399 | C | LEU | A | 427 | 101.255 | 31.734 | 21.294 | 1.00 | 17.83 | C | |
| ATOM | 3400 | O | LEU | A | 427 | 101.910 | 32.201 | 22.246 | 1.00 | 17.76 | O | |
| ATOM | 3401 | CB | LEU | A | 427 | 100.193 | 33.241 | 19.561 | 1.00 | 22.15 | C | |
| ATOM | 3402 | CG | LEU | A | 427 | 99.164 | 33.289 | 18.408 | 1.00 | 27.60 | C | |
| ATOM | 3403 | CD1 | LEU | A | 427 | 97.888 | 32.599 | 18.752 | 1.00 | 28.50 | C | |
| ATOM | 3404 | CD2 | LEU | A | 427 | 99.026 | 34.715 | 17.895 | 1.00 | 26.30 | C | |
| ATOM | 3405 | N | PRO | A | 428 | 101.665 | 30.649 | 20.645 | 1.00 | 17.74 | N | |
| ATOM | 3406 | CA | PRO | A | 428 | 102.879 | 29.954 | 21.041 | 1.00 | 21.49 | C | |
| ATOM | 3407 | C | PRO | A | 428 | 104.137 | 30.525 | 20.445 | 1.00 | 21.45 | C | |
| ATOM | 3408 | O | PRO | A | 428 | 104.814 | 29.868 | 19.654 | 1.00 | 23.23 | O | |
| ATOM | 3409 | CB | PRO | A | 428 | 102.618 | 28.505 | 20.593 | 1.00 | 17.24 | C | |
| ATOM | 3410 | CG | PRO | A | 428 | 101.748 | 28.706 | 19.275 | 1.00 | 18.87 | C | |
| ATOM | 3411 | CD | PRO | A | 428 | 100.943 | 30.009 | 19.532 | 1.00 | 18.18 | C | |
| ATOM | 3412 | N | THR | A | 429 | 104.491 | 31.725 | 20.870 | 1.00 | 18.87 | N | |
| ATOM | 3413 | CA | THR | A | 429 | 105.688 | 32.345 | 20.317 | 1.00 | 20.98 | C | |
| ATOM | 3414 | C | THR | A | 429 | 106.866 | 32.119 | 21.236 | 1.00 | 21.62 | C | |
| ATOM | 3415 | O | THR | A | 429 | 106.711 | 32.179 | 22.435 | 1.00 | 24.02 | O | |
| ATOM | 3416 | CB | THR | A | 429 | 105.508 | 33.920 | 20.196 | 1.00 | 24.02 | C | |
| ATOM | 3417 | OG1 | THR | A | 429 | 104.883 | 34.388 | 21.394 | 1.00 | 23.58 | O | |
| ATOM | 3418 | CG2 | THR | A | 429 | 104.642 | 34.315 | 19.005 | 1.00 | 21.06 | C | |
| ATOM | 3419 | N | PRO | A | 430 | 108.055 | 31.846 | 20.676 | 1.00 | 23.16 | N | |
| ATOM | 3420 | CA | PRO | A | 430 | 109.281 | 31.638 | 21.461 | 1.00 | 25.95 | C | |
| ATOM | 3421 | C | PRO | A | 430 | 109.782 | 33.003 | 21.900 | 1.00 | 27.94 | C | |
| ATOM | 3422 | O | PRO | A | 430 | 109.184 | 34.023 | 21.560 | 1.00 | 23.22 | O | |
| ATOM | 3423 | CB | PRO | A | 430 | 110.233 | 30.965 | 20.466 | 1.00 | 23.91 | C | |
| ATOM | 3424 | CG | PRO | A | 430 | 109.778 | 31.495 | 19.113 | 1.00 | 25.65 | C | |
| ATOM | 3425 | CD | PRO | A | 430 | 108.258 | 31.521 | 19.255 | 1.00 | 25.14 | C | |
| ATOM | 3426 | N | ALA | A | 431 | 110.885 | 33.022 | 22.650 | 1.00 | 27.17 | N | |
| ATOM | 3427 | CA | ALA | A | 431 | 111.414 | 34.289 | 23.151 | 1.00 | 27.49 | C | |
| ATOM | 3428 | C | ALA | A | 431 | 111.787 | 35.293 | 22.052 | 1.00 | 25.89 | C | |
| ATOM | 3429 | O | ALA | A | 431 | 112.258 | 34.923 | 20.969 | 1.00 | 24.83 | O | |
| ATOM | 3430 | CB | ALA | A | 431 | 112.625 | 34.011 | 24.028 | 1.00 | 24.28 | C | |
| ATOM | 3431 | N | ARG | A | 432 | 111.572 | 36.570 | 22.355 | 1.00 | 28.54 | N | |
| ATOM | 3432 | CA | ARG | A | 432 | 111.911 | 37.680 | 21.458 | 1.00 | 30.43 | C | |
| ATOM | 3433 | C | ARG | A | 432 | 111.367 | 37.638 | 20.026 | 1.00 | 30.67 | C | |
| ATOM | 3434 | O | ARG | A | 432 | 111.992 | 38.221 | 19.114 | 1.00 | 31.09 | O | |
| ATOM | 3435 | CB | ARG | A | 432 | 113.434 | 37.840 | 21.356 | 1.00 | 34.94 | C | |
| ATOM | 3436 | CG | ARG | A | 432 | 114.225 | 37.832 | 22.656 | 1.00 | 38.43 | C | |
| ATOM | 3437 | CD | ARG | A | 432 | 115.598 | 37.159 | 22.398 | 1.00 | 43.63 | C | |
| ATOM | 3438 | NE | ARG | A | 432 | 115.848 | 36.054 | 23.320 | 1.00 | 46.50 | N | |
| ATOM | 3439 | CZ | ARG | A | 432 | 116.796 | 35.136 | 23.156 | 1.00 | 48.68 | C | |
| ATOM | 3440 | NH1 | ARG | A | 432 | 117.586 | 35.193 | 22.099 | 1.00 | 50.18 | N | |
| ATOM | 3441 | NH2 | ARG | A | 432 | 116.955 | 34.160 | 24.051 | 1.00 | 51.79 | N | |
| ATOM | 3442 | N | VAL | A | 433 | 110.241 | 36.970 | 19.797 | 1.00 | 21.77 | N | |
| ATOM | 3443 | CA | VAL | A | 433 | 109.677 | 36.906 | 18.459 | 1.00 | 26.93 | C | |
| ATOM | 3444 | C | VAL | A | 433 | 108.329 | 37.595 | 18.423 | 1.00 | 30.21 | C | |
| ATOM | 3445 | O | VAL | A | 433 | 107.530 | 37.377 | 19.314 | 1.00 | 32.17 | O | |
| ATOM | 3446 | CB | VAL | A | 433 | 109.511 | 35.405 | 17.998 | 1.00 | 26.51 | C | |
| ATOM | 3447 | CG1 | VAL | A | 433 | 108.356 | 35.240 | 17.018 | 1.00 | 31.84 | C | |
| ATOM | 3448 | CG2 | VAL | A | 433 | 110.847 | 34.930 | 17.320 | 1.00 | 23.21 | C | |
| ATOM | 3449 | N | GLN | A | 434 | 108.095 | 38.435 | 17.406 | 1.00 | 29.12 | N | |
| ATOM | 3450 | CA | GLN | A | 434 | 106.808 | 39.104 | 17.222 | 1.00 | 30.27 | C | |
| ATOM | 3451 | C | GLN | A | 434 | 106.286 | 38.853 | 15.785 | 1.00 | 31.28 | C | |
| ATOM | 3452 | O | GLN | A | 434 | 107.075 | 38.756 | 14.827 | 1.00 | 29.65 | O | |
| ATOM | 3453 | CB | GLN | A | 434 | 106.947 | 40.619 | 17.519 | 1.00 | 31.56 | C | |
| ATOM | 3454 | CG | GLN | A | 434 | 107.404 | 40.933 | 18.969 | 1.00 | 36.69 | C | |
| ATOM | 3455 | CD | GLN | A | 434 | 107.250 | 42.427 | 19.376 | 1.00 | 40.55 | C | |
| ATOM | 3456 | OE1 | GLN | A | 434 | 106.127 | 42.938 | 19.461 | 1.00 | 36.93 | O | |
| ATOM | 3457 | NE2 | GLN | A | 434 | 108.383 | 43.116 | 19.627 | 1.00 | 40.09 | N | |
| ATOM | 3458 | N | LEU | A | 435 | 104.965 | 38.736 | 15.621 | 1.00 | 30.10 | N | |
| ATOM | 3459 | CA | LEU | A | 435 | 104.378 | 38.469 | 14.308 | 1.00 | 30.39 | C | |
| ATOM | 3460 | C | LEU | A | 435 | 103.920 | 39.711 | 13.523 | 1.00 | 30.20 | C | |
| ATOM | 3461 | O | LEU | A | 435 | 103.413 | 40.628 | 14.126 | 1.00 | 33.46 | O | |
| ATOM | 3462 | CB | LEU | A | 435 | 103.181 | 37.533 | 14.506 | 1.00 | 33.74 | C | |
| ATOM | 3463 | CG | LEU | A | 435 | 103.581 | 36.311 | 15.342 | 1.00 | 35.93 | C | |
| ATOM | 3464 | CD1 | LEU | A | 435 | 102.347 | 35.560 | 15.799 | 1.00 | 35.75 | C | |
| ATOM | 3465 | CD2 | LEU | A | 435 | 104.515 | 35.433 | 14.487 | 1.00 | 36.78 | C | |
| ATOM | 3466 | N | TYR | A | 436 | 104.083 | 39.739 | 12.190 | 1.00 | 29.33 | N | |
| ATOM | 3467 | CA | TYR | A | 436 | 103.605 | 40.868 | 11.383 | 1.00 | 27.87 | C | |
| ATOM | 3468 | C | TYR | A | 436 | 103.174 | 40.410 | 10.000 | 1.00 | 30.05 | C | |
| ATOM | 3469 | O | TYR | A | 436 | 103.297 | 39.212 | 9.676 | 1.00 | 28.38 | O | |
| ATOM | 3470 | CB | TYR | A | 436 | 104.644 | 42.022 | 11.318 | 1.00 | 27.89 | C | |
| ATOM | 3471 | CG | TYR | A | 436 | 105.870 | 41.805 | 10.472 | 1.00 | 25.19 | C | |
| ATOM | 3472 | CD1 | TYR | A | 436 | 106.912 | 41.009 | 10.917 | 1.00 | 28.80 | C | |
| ATOM | 3473 | CD2 | TYR | A | 436 | 105.981 | 42.383 | 9.198 | 1.00 | 26.32 | C | |
| ATOM | 3474 | CE1 | TYR | A | 436 | 108.025 | 40.787 | 10.108 | 1.00 | 25.24 | C | |
| ATOM | 3475 | CE2 | TYR | A | 436 | 107.059 | 42.175 | 8.392 | 1.00 | 23.20 | C | |
| ATOM | 3476 | CZ | TYR | A | 436 | 108.093 | 41.360 | 8.848 | 1.00 | 24.73 | C | |
| ATOM | 3477 | OH | TYR | A | 436 | 109.149 | 41.076 | 8.040 | 1.00 | 25.47 | O | |
| ATOM | 3478 | N | AASN | A | 437 | 102.667 | 41.343 | 9.190 | 0.50 | 28.37 | N | |
| ATOM | 3479 | N | BASN | A | 437 | 102.668 | 41.340 | 9.186 | 0.50 | 26.79 | N | |
| ATOM | 3480 | CA | AASN | A | 437 | 102.179 | 41.039 | 7.848 | 0.50 | 29.41 | C | |
| ATOM | 3481 | CA | BASN | A | 437 | 102.204 | 41.033 | 7.832 | 0.50 | 26.48 | C | |
| ATOM | 3482 | C | AASN | A | 437 | 101.207 | 39.872 | 7.930 | 0.50 | 30.78 | C | |
| ATOM | 3483 | C | BASN | A | 437 | 101.216 | 39.866 | 7.930 | 0.50 | 29.14 | C | |
| ATOM | 3484 | O | AASN | A | 437 | 101.254 | 38.968 | 7.105 | 0.50 | 29.23 | O | |
| ATOM | 3485 | O | BASN | A | 437 | 101.259 | 38.955 | 7.112 | 0.50 | 27.72 | O | |
| ATOM | 3486 | CB | AASN | A | 437 | 103.323 | 40.649 | 6.912 | 0.50 | 32.27 | C | |
| ATOM | 3487 | CB | BASN | A | 437 | 103.400 | 40.639 | 6.942 | 0.50 | 26.39 | C | |
| ATOM | 3488 | CG | AASN | A | 437 | 104.118 | 41.835 | 6.442 | 0.50 | 36.64 | C | |
| ATOM | 3489 | CG | BASN | A | 437 | 103.018 | 40.367 | 5.487 | 0.50 | 25.88 | C | |
| ATOM | 3490 | OD1 | AASN | A | 437 | 104.019 | 42.932 | 7.010 | 0.50 | 39.42 | O | |
| ATOM | 3491 | OD1 | BASN | A | 437 | 103.785 | 39.742 | 4.741 | 0.50 | 27.37 | O | |
| ATOM | 3492 | ND2 | AASN | A | 437 | 104.927 | 41.628 | 5.399 | 0.50 | 34.66 | N | |
| ATOM | 3493 | ND2 | BASN | A | 437 | 101.862 | 40.842 | 5.071 | 0.50 | 26.13 | N | |
| ATOM | 3494 | N | VAL | A | 438 | 100.316 | 39.913 | 8.915 | 1.00 | 30.56 | N | |
| ATOM | 3495 | CA | VAL | A | 438 | 99.348 | 38.823 | 9.108 | 1.00 | 32.47 | C | |
| ATOM | 3496 | C | VAL | A | 438 | 98.194 | 38.788 | 8.127 | 1.00 | 34.47 | C | |
| ATOM | 3497 | O | VAL | A | 438 | 97.598 | 39.810 | 7.761 | 1.00 | 32.50 | O | |
| ATOM | 3498 | CB | VAL | A | 438 | 98.776 | 38.847 | 10.525 | 1.00 | 31.87 | C | |
| ATOM | 3499 | CG1 | VAL | A | 438 | 99.882 | 38.858 | 11.518 | 1.00 | 31.13 | C | |
| ATOM | 3500 | CG2 | VAL | A | 438 | 97.889 | 40.072 | 10.724 | 1.00 | 36.41 | C | |
| ATOM | 3501 | N | VAL | A | 439 | 97.905 | 37.588 | 7.652 | 1.00 | 31.32 | N | |
| ATOM | 3502 | CA | VAL | A | 439 | 96.800 | 37.406 | 6.759 | 1.00 | 36.11 | C | |
| ATOM | 3503 | C | VAL | A | 439 | 95.986 | 36.241 | 7.345 | 1.00 | 37.14 | C | |
| ATOM | 3504 | O | VAL | A | 439 | 96.522 | 35.377 | 8.071 | 1.00 | 35.65 | O | |
| ATOM | 3505 | CB | VAL | A | 439 | 97.273 | 37.104 | 5.337 | 1.00 | 38.19 | C | |
| ATOM | 3506 | CG1 | VAL | A | 439 | 98.226 | 35.915 | 5.367 | 1.00 | 43.13 | C | |
| ATOM | 3507 | CG2 | VAL | A | 439 | 96.078 | 36.833 | 4.426 | 1.00 | 41.07 | C | |
| ATOM | 3508 | N | LEU | A | 440 | 94.685 | 36.266 | 7.089 | 1.00 | 34.94 | N | |
| ATOM | 3509 | CA | LEU | A | 440 | 93.828 | 35.207 | 7.575 | 1.00 | 36.32 | C | |
| ATOM | 3510 | C | LEU | A | 440 | 92.984 | 34.651 | 6.445 | 1.00 | 37.21 | C | |
| ATOM | 3511 | O | LEU | A | 440 | 92.425 | 35.383 | 5.629 | 1.00 | 34.43 | O | |
| ATOM | 3512 | CB | LEU | A | 440 | 92.904 | 35.710 | 8.676 | 1.00 | 36.50 | C | |
| ATOM | 3513 | CG | LEU | A | 440 | 91.658 | 34.812 | 8.872 | 1.00 | 38.03 | C | |
| ATOM | 3514 | CD1 | LEU | A | 440 | 92.075 | 33.435 | 9.471 | 1.00 | 37.31 | C | |
| ATOM | 3515 | CD2 | LEU | A | 440 | 90.640 | 35.509 | 9.775 | 1.00 | 38.88 | C | |
| ATOM | 3516 | N | GLN | A | 441 | 92.916 | 33.341 | 6.386 | 1.00 | 34.60 | N | |
| ATOM | 3517 | CA | GLN | A | 441 | 92.098 | 32.713 | 5.409 | 1.00 | 35.49 | C | |
| ATOM | 3518 | C | GLN | A | 441 | 91.343 | 31.610 | 6.155 | 1.00 | 35.54 | C | |
| ATOM | 3519 | O | GLN | A | 441 | 91.944 | 30.698 | 6.754 | 1.00 | 34.07 | O | |
| ATOM | 3520 | CB | GLN | A | 441 | 92.929 | 32.180 | 4.207 | 1.00 | 38.29 | C | |
| ATOM | 3521 | CG | GLN | A | 441 | 94.232 | 31.383 | 4.512 | 1.00 | 43.75 | C | |
| ATOM | 3522 | CD | GLN | A | 441 | 94.977 | 30.862 | 3.238 | 1.00 | 45.12 | C | |
| ATOM | 3523 | OE1 | GLN | A | 441 | 94.466 | 30.019 | 2.499 | 1.00 | 39.90 | O | |
| ATOM | 3524 | NE2 | GLN | A | 441 | 96.210 | 31.368 | 3.013 | 1.00 | 46.48 | N | |
| ATOM | 3525 | N | PRO | A | 442 | 90.017 | 31.731 | 6.220 | 1.00 | 33.67 | N | |
| ATOM | 3526 | CA | PRO | A | 442 | 89.398 | 30.621 | 6.927 | 1.00 | 30.02 | C | |
| ATOM | 3527 | C | PRO | A | 442 | 89.342 | 29.430 | 5.977 | 1.00 | 29.82 | C | |
| ATOM | 3528 | O | PRO | A | 442 | 89.255 | 29.584 | 4.766 | 1.00 | 29.81 | O | |
| ATOM | 3529 | CB | PRO | A | 442 | 88.000 | 31.156 | 7.273 | 1.00 | 31.27 | C | |
| ATOM | 3530 | CG | PRO | A | 442 | 88.226 | 32.645 | 7.331 | 1.00 | 34.90 | C | |
| ATOM | 3531 | CD | PRO | A | 442 | 89.133 | 32.918 | 6.210 | 1.00 | 31.78 | C | |
| ATOM | 3532 | N | HIS | A | 443 | 89.457 | 28.240 | 6.525 | 1.00 | 26.17 | N | |
| ATOM | 3533 | CA | HIS | A | 443 | 89.329 | 27.030 | 5.749 | 1.00 | 25.42 | C | |
| ATOM | 3534 | C | HIS | A | 443 | 88.255 | 26.234 | 6.431 | 1.00 | 23.53 | C | |
| ATOM | 3535 | O | HIS | A | 443 | 87.791 | 26.574 | 7.509 | 1.00 | 25.93 | O | |
| ATOM | 3536 | CB | HIS | A | 443 | 90.603 | 26.183 | 5.734 | 1.00 | 22.58 | C | |
| ATOM | 3537 | CG | HIS | A | 443 | 91.666 | 26.691 | 4.814 | 1.00 | 26.96 | C | |
| ATOM | 3538 | ND1 | HIS | A | 443 | 92.222 | 25.911 | 3.820 | 1.00 | 27.56 | N | |
| ATOM | 3539 | CD2 | HIS | A | 443 | 92.263 | 27.904 | 4.725 | 1.00 | 25.93 | C | |
| ATOM | 3540 | CE1 | HIS | A | 443 | 93.114 | 26.629 | 3.152 | 1.00 | 28.01 | C | |
| ATOM | 3541 | NE2 | HIS | A | 443 | 93.159 | 27.838 | 3.678 | 1.00 | 29.23 | N | |
| ATOM | 3542 | N | GLN | A | 444 | 87.854 | 25.147 | 5.792 | 1.00 | 26.75 | N | |
| ATOM | 3543 | CA | GLN | A | 444 | 86.854 | 24.293 | 6.415 | 1.00 | 26.51 | C | |
| ATOM | 3544 | C | GLN | A | 444 | 87.498 | 23.631 | 7.672 | 1.00 | 24.96 | C | |
| ATOM | 3545 | O | GLN | A | 444 | 88.513 | 22.934 | 7.578 | 1.00 | 23.07 | O | |
| ATOM | 3546 | CB | GLN | A | 444 | 86.401 | 23.210 | 5.428 | 1.00 | 29.64 | C | |
| ATOM | 3547 | CG | GLN | A | 444 | 85.238 | 22.388 | 5.936 | 1.00 | 35.33 | C | |
| ATOM | 3548 | CD | GLN | A | 444 | 83.963 | 23.235 | 6.089 | 1.00 | 41.12 | C | |
| ATOM | 3549 | OE1 | GLN | A | 444 | 83.128 | 22.973 | 6.948 | 1.00 | 43.39 | O | |
| ATOM | 3550 | NE2 | GLN | A | 444 | 83.816 | 24.245 | 5.241 | 1.00 | 38.86 | N | |
| ATOM | 3551 | N | ASN | A | 445 | 86.895 | 23.873 | 8.829 | 1.00 | 20.79 | N | |
| ATOM | 3552 | CA | ASN | A | 445 | 87.343 | 23.289 | 10.111 | 1.00 | 20.60 | C | |
| ATOM | 3553 | C | ASN | A | 445 | 88.584 | 23.879 | 10.766 | 1.00 | 20.05 | C | |
| ATOM | 3554 | O | ASN | A | 445 | 89.017 | 23.403 | 11.832 | 1.00 | 16.72 | O | |
| ATOM | 3555 | CB | ASN | A | 445 | 87.498 | 21.763 | 9.997 | 1.00 | 22.62 | C | |
| ATOM | 3556 | CG | ASN | A | 445 | 86.214 | 21.099 | 9.539 | 1.00 | 26.79 | C | |
| ATOM | 3557 | OD1 | ASN | A | 445 | 86.236 | 20.053 | 8.868 | 1.00 | 29.77 | O | |
| ATOM | 3558 | ND2 | ASN | A | 445 | 85.094 | 21.707 | 9.884 | 1.00 | 19.91 | N | |
| ATOM | 3559 | N | PHE | A | 446 | 89.151 | 24.900 | 10.148 | 1.00 | 19.07 | N | |
| ATOM | 3560 | CA | PHE | A | 446 | 90.285 | 25.573 | 10.798 | 1.00 | 17.78 | C | |
| ATOM | 3561 | C | PHE | A | 446 | 90.559 | 26.925 | 10.190 | 1.00 | 20.51 | C | |
| ATOM | 3562 | O | PHE | A | 446 | 90.317 | 27.167 | 9.011 | 1.00 | 20.33 | O | |
| ATOM | 3563 | CB | PHE | A | 446 | 91.613 | 24.759 | 10.748 | 1.00 | 16.32 | C | |
| ATOM | 3564 | CG | PHE | A | 446 | 92.191 | 24.549 | 9.336 | 1.00 | 21.48 | C | |
| ATOM | 3565 | CD1 | PHE | A | 446 | 91.672 | 23.525 | 8.472 | 1.00 | 20.65 | C | |
| ATOM | 3566 | CD2 | PHE | A | 446 | 93.291 | 25.301 | 8.909 | 1.00 | 20.96 | C | |
| ATOM | 3567 | CE1 | PHE | A | 446 | 92.252 | 23.264 | 7.227 | 1.00 | 20.84 | C | |
| ATOM | 3568 | CE2 | PHE | A | 446 | 93.889 | 25.046 | 7.634 | 1.00 | 23.60 | C | |
| ATOM | 3569 | CZ | PHE | A | 446 | 93.366 | 24.026 | 6.799 | 1.00 | 23.81 | C | |
| ATOM | 3570 | N | LEU | A | 447 | 91.118 | 27.787 | 11.022 | 1.00 | 18.73 | N | |
| ATOM | 3571 | CA | LEU | A | 447 | 91.515 | 29.117 | 10.589 | 1.00 | 19.06 | C | |
| ATOM | 3572 | C | LEU | A | 447 | 93.019 | 29.026 | 10.292 | 1.00 | 21.79 | C | |
| ATOM | 3573 | O | LEU | A | 447 | 93.777 | 28.502 | 11.118 | 1.00 | 19.94 | O | |
| ATOM | 3574 | CB | LEU | A | 447 | 91.323 | 30.083 | 11.736 | 1.00 | 20.93 | C | |
| ATOM | 3575 | CG | LEU | A | 447 | 90.394 | 31.268 | 11.561 | 1.00 | 34.61 | C | |
| ATOM | 3576 | CD1 | LEU | A | 447 | 89.167 | 30.903 | 10.734 | 1.00 | 31.44 | C | |
| ATOM | 3577 | CD2 | LEU | A | 447 | 90.021 | 31.775 | 12.972 | 1.00 | 30.45 | C | |
| ATOM | 3578 | N | LEU | A | 448 | 93.433 | 29.576 | 9.149 | 1.00 | 19.34 | N | |
| ATOM | 3579 | CA | LEU | A | 448 | 94.839 | 29.605 | 8.781 | 1.00 | 22.65 | C | |
| ATOM | 3580 | C | LEU | A | 448 | 95.354 | 31.026 | 8.837 | 1.00 | 26.10 | C | |
| ATOM | 3581 | O | LEU | A | 448 | 94.854 | 31.881 | 8.103 | 1.00 | 28.78 | O | |
| ATOM | 3582 | CB | LEU | A | 448 | 95.071 | 29.087 | 7.351 | 1.00 | 23.58 | C | |
| ATOM | 3583 | CG | LEU | A | 448 | 96.540 | 28.992 | 6.896 | 1.00 | 24.08 | C | |
| ATOM | 3584 | CD1 | LEU | A | 448 | 97.352 | 28.054 | 7.852 | 1.00 | 26.56 | C | |
| ATOM | 3585 | CD2 | LEU | A | 448 | 96.611 | 28.401 | 5.444 | 1.00 | 25.75 | C | |
| ATOM | 3586 | N | PHE | A | 449 | 96.353 | 31.252 | 9.690 | 1.00 | 22.16 | N | |
| ATOM | 3587 | CA | PHE | A | 449 | 97.002 | 32.548 | 9.818 | 1.00 | 27.70 | C | |
| ATOM | 3588 | C | PHE | A | 449 | 98.413 | 32.464 | 9.259 | 1.00 | 27.30 | C | |
| ATOM | 3589 | O | PHE | A | 449 | 99.200 | 31.625 | 9.668 | 1.00 | 30.52 | O | |
| ATOM | 3590 | CB | PHE | A | 449 | 97.076 | 32.963 | 11.265 | 1.00 | 31.43 | C | |
| ATOM | 3591 | CG | PHE | A | 449 | 95.844 | 33.691 | 11.737 | 1.00 | 35.96 | C | |
| ATOM | 3592 | CD1 | PHE | A | 449 | 95.551 | 34.973 | 11.261 | 1.00 | 36.95 | C | |
| ATOM | 3593 | CD2 | PHE | A | 449 | 94.962 | 33.090 | 12.643 | 1.00 | 40.57 | C | |
| ATOM | 3594 | CE1 | PHE | A | 449 | 94.400 | 35.652 | 11.664 | 1.00 | 36.95 | C | |
| ATOM | 3595 | CE2 | PHE | A | 449 | 93.804 | 33.768 | 13.055 | 1.00 | 38.38 | C | |
| ATOM | 3596 | CZ | PHE | A | 449 | 93.536 | 35.058 | 12.557 | 1.00 | 36.99 | C | |
| ATOM | 3597 | N | GLY | A | 450 | 98.679 | 33.319 | 8.293 | 1.00 | 27.97 | N | |
| ATOM | 3598 | CA | GLY | A | 450 | 99.989 | 33.412 | 7.652 | 1.00 | 29.22 | C | |
| ATOM | 3599 | C | GLY | A | 450 | 100.637 | 34.680 | 8.164 | 1.00 | 29.80 | C | |
| ATOM | 3600 | O | GLY | A | 450 | 99.952 | 35.700 | 8.345 | 1.00 | 29.78 | O | |
| ATOM | 3601 | N | ALA | A | 451 | 101.945 | 34.627 | 8.394 | 1.00 | 29.19 | N | |
| ATOM | 3602 | CA | ALA | A | 451 | 102.642 | 35.773 | 8.944 | 1.00 | 25.12 | C | |
| ATOM | 3603 | C | ALA | A | 451 | 104.145 | 35.754 | 8.809 | 1.00 | 27.49 | C | |
| ATOM | 3604 | O | ALA | A | 451 | 104.737 | 34.686 | 8.622 | 1.00 | 21.59 | O | |
| ATOM | 3605 | CB | ALA | A | 451 | 102.327 | 35.877 | 10.353 | 1.00 | 24.08 | C | |
| ATOM | 3606 | N | ASP | A | 452 | 104.752 | 36.953 | 8.898 | 1.00 | 25.36 | N | |
| ATOM | 3607 | CA | ASP | A | 452 | 106.208 | 37.056 | 8.886 | 1.00 | 25.80 | C | |
| ATOM | 3608 | C | ASP | A | 452 | 106.617 | 37.246 | 10.329 | 1.00 | 24.82 | C | |
| ATOM | 3609 | O | ASP | A | 452 | 105.785 | 37.610 | 11.171 | 1.00 | 27.57 | O | |
| ATOM | 3610 | CB | ASP | A | 452 | 106.684 | 38.187 | 7.945 | 1.00 | 26.54 | C | |
| ATOM | 3611 | CG | ASP | A | 452 | 106.427 | 37.838 | 6.481 | 1.00 | 24.14 | C | |
| ATOM | 3612 | OD1 | ASP | A | 452 | 106.390 | 36.622 | 6.133 | 1.00 | 25.74 | O | |
| ATOM | 3613 | OD2 | ASP | A | 452 | 106.226 | 38.756 | 5.650 | 1.00 | 25.90 | O | |
| ATOM | 3614 | N | AVAL | A | 453 | 107.879 | 36.957 | 10.609 | 0.50 | 22.35 | N | |
| ATOM | 3615 | N | BVAL | A | 453 | 107.875 | 36.991 | 10.664 | 0.50 | 22.88 | N | |
| ATOM | 3616 | CA | AVAL | A | 453 | 108.427 | 37.025 | 11.938 | 0.50 | 21.03 | C | |
| ATOM | 3617 | CA | BVAL | A | 453 | 108.305 | 37.131 | 12.042 | 0.50 | 22.64 | C | |
| ATOM | 3618 | C | AVAL | A | 453 | 109.584 | 38.001 | 12.019 | 0.50 | 22.27 | C | |
| ATOM | 3619 | C | BVAL | A | 453 | 109.499 | 38.050 | 12.221 | 0.50 | 24.32 | C | |
| ATOM | 3620 | O | AVAL | A | 453 | 110.375 | 38.146 | 11.080 | 0.50 | 19.85 | O | |
| ATOM | 3621 | O | BVAL | A | 453 | 110.375 | 38.141 | 11.356 | 0.50 | 23.04 | O | |
| ATOM | 3622 | CB | AVAL | A | 453 | 108.943 | 35.607 | 12.380 | 0.50 | 23.45 | C | |
| ATOM | 3623 | CB | BVAL | A | 453 | 108.642 | 35.735 | 12.681 | 0.50 | 24.58 | C | |
| ATOM | 3624 | CG1 | AVAL | A | 453 | 109.945 | 35.730 | 13.512 | 0.50 | 22.91 | C | |
| ATOM | 3625 | CG1 | BVAL | A | 453 | 109.461 | 34.898 | 11.720 | 0.50 | 22.55 | C | |
| ATOM | 3626 | CG2 | AVAL | A | 453 | 107.780 | 34.728 | 12.789 | 0.50 | 22.07 | C | |
| ATOM | 3627 | CG2 | BVAL | A | 453 | 109.414 | 35.926 | 13.994 | 0.50 | 25.79 | C | |
| ATOM | 3628 | N | AVAL | A | 454 | 109.655 | 38.697 | 13.144 | 0.50 | 21.67 | N | |
| ATOM | 3629 | N | BVAL | A | 454 | 109.510 | 38.758 | 13.344 | 0.50 | 24.28 | N | |
| ATOM | 3630 | CA | AVAL | A | 454 | 110.747 | 39.611 | 13.387 | 0.50 | 22.52 | C | |
| ATOM | 3631 | CA | BVAL | A | 454 | 110.608 | 39.646 | 13.667 | 0.50 | 25.20 | C | |
| ATOM | 3632 | C | AVAL | A | 454 | 111.300 | 39.164 | 14.747 | 0.50 | 22.17 | C | |
| ATOM | 3633 | C | BVAL | A | 454 | 111.308 | 39.127 | 14.895 | 0.50 | 24.52 | C | |
| ATOM | 3634 | O | AVAL | A | 454 | 110.539 | 38.979 | 15.702 | 0.50 | 24.20 | O | |
| ATOM | 3635 | O | BVAL | A | 454 | 110.673 | 38.901 | 15.931 | 0.50 | 26.42 | O | |
| ATOM | 3636 | CB | AVAL | A | 454 | 110.280 | 41.128 | 13.361 | 0.50 | 20.96 | C | |
| ATOM | 3637 | CB | BVAL | A | 454 | 110.151 | 41.103 | 13.985 | 0.50 | 26.07 | C | |
| ATOM | 3638 | CG1 | AVAL | A | 454 | 109.020 | 41.343 | 14.207 | 0.50 | 16.41 | C | |
| ATOM | 3639 | CG1 | BVAL | A | 454 | 111.278 | 41.856 | 14.686 | 0.50 | 26.68 | C | |
| ATOM | 3640 | CG2 | AVAL | A | 454 | 111.423 | 42.001 | 13.832 | 0.50 | 23.65 | C | |
| ATOM | 3641 | CG2 | BVAL | A | 454 | 109.787 | 41.807 | 12.733 | 0.50 | 21.85 | C | |
| ATOM | 3642 | N | TYR | A | 455 | 112.615 | 38.928 | 14.786 | 1.00 | 22.38 | N | |
| ATOM | 3643 | CA | TYR | A | 455 | 113.375 | 38.470 | 15.950 | 1.00 | 22.98 | C | |
| ATOM | 3644 | C | TYR | A | 455 | 114.315 | 39.571 | 16.504 | 1.00 | 29.35 | C | |
| ATOM | 3645 | O | TYR | A | 455 | 114.931 | 40.303 | 15.742 | 1.00 | 27.45 | O | |
| ATOM | 3646 | CB | TYR | A | 455 | 114.256 | 37.287 | 15.555 | 1.00 | 26.26 | C | |
| ATOM | 3647 | CG | TYR | A | 455 | 115.206 | 36.847 | 16.640 | 1.00 | 24.63 | C | |
| ATOM | 3648 | CD1 | TYR | A | 455 | 114.748 | 36.180 | 17.796 | 1.00 | 30.39 | C | |
| ATOM | 3649 | CD2 | TYR | A | 455 | 116.578 | 37.050 | 16.515 | 1.00 | 27.09 | C | |
| ATOM | 3650 | CE1 | TYR | A | 455 | 115.653 | 35.730 | 18.784 | 1.00 | 30.67 | C | |
| ATOM | 3651 | CE2 | TYR | A | 455 | 117.471 | 36.596 | 17.494 | 1.00 | 28.49 | C | |
| ATOM | 3652 | CZ | TYR | A | 455 | 117.021 | 35.943 | 18.615 | 1.00 | 31.62 | C | |
| ATOM | 3653 | OH | TYR | A | 455 | 117.943 | 35.491 | 19.558 | 1.00 | 32.04 | O | |
| ATOM | 3654 | N | LYS | A | 456 | 114.453 | 39.685 | 17.822 | 1.00 | 34.22 | N | |
| ATOM | 3655 | CA | LYS | A | 456 | 115.368 | 40.710 | 18.335 | 1.00 | 40.96 | C | |
| ATOM | 3656 | C | LYS | A | 456 | 116.207 | 40.240 | 19.531 | 1.00 | 44.03 | C | |
| ATOM | 3657 | O | LYS | A | 456 | 117.455 | 40.143 | 19.415 | 1.00 | 45.32 | O | |
| ATOM | 3658 | CB | LYS | A | 456 | 114.570 | 41.967 | 18.679 | 1.00 | 40.55 | C | |
| ATOM | 3659 | CG | LYS | A | 456 | 113.678 | 42.482 | 17.510 | 1.00 | 39.39 | C | |
| ATOM | 3660 | CD | LYS | A | 456 | 112.429 | 43.272 | 17.993 | 1.00 | 44.56 | C | |
| ATOM | 3661 | CE | LYS | A | 456 | 111.457 | 42.393 | 18.837 | 1.00 | 43.77 | C | |
| ATOM | 3662 | NZ | LYS | A | 456 | 110.868 | 41.202 | 18.097 | 1.00 | 35.09 | N | |
| ATOM | 3663 | OXT | LYS | A | 456 | 115.595 | 39.955 | 20.584 | 1.00 | 49.29 | O | |
| TER | 3664 | | LYS | A | 456 | | | | | | | |
| HETATM | 3665 | C1 | PC2 | | 577 | 121.235 | 32.739 | 19.104 | 1.00 | 59.16 | C | |
| HETATM | 3666 | C2 | PC2 | | 577 | 120.356 | 31.500 | 18.816 | 1.00 | 57.07 | C | |
| HETATM | 3667 | C3 | PC2 | | 577 | 119.568 | 31.650 | 17.503 | 1.00 | 56.79 | C | |
| HETATM | 3668 | C4 | PC2 | | 577 | 121.205 | 33.400 | 23.182 | 1.00 | 61.06 | C | |
| HETATM | 3669 | C5 | PC2 | | 577 | 120.728 | 33.401 | 24.674 | 1.00 | 60.65 | C | |
| HETATM | 3670 | C6 | PC2 | | 577 | 122.072 | 35.346 | 25.394 | 1.00 | 59.92 | C | |
| HETATM | 3671 | C7 | PC2 | | 577 | 120.292 | 34.461 | 26.780 | 1.00 | 60.68 | C | |
| HETATM | 3672 | C8 | PC2 | | 577 | 119.736 | 35.662 | 24.847 | 1.00 | 61.06 | C | |
| HETATM | 3673 | C11 | PC2 | | 577 | 117.221 | 32.516 | 17.064 | 1.00 | 57.74 | C | |
| HETATM | 3674 | C12 | PC2 | | 577 | 117.198 | 31.721 | 15.738 | 1.00 | 54.85 | C | |
| HETATM | 3675 | C13 | PC2 | | 577 | 116.145 | 32.257 | 14.732 | 1.00 | 50.33 | C | |
| HETATM | 3676 | C14 | PC2 | | 577 | 114.692 | 31.939 | 15.150 | 1.00 | 49.07 | C | |
| HETATM | 3677 | C15 | PC2 | | 577 | 113.654 | 33.001 | 14.715 | 1.00 | 48.44 | C | |
| HETATM | 3678 | C16 | PC2 | | 577 | 113.310 | 32.887 | 13.223 | 1.00 | 48.78 | C | |
| HETATM | 3679 | C17 | PC2 | | 577 | 111.846 | 32.603 | 12.954 | 1.00 | 46.01 | C | |
| HETATM | 3680 | C18 | PC2 | | 577 | 111.601 | 31.175 | 12.380 | 1.00 | 45.14 | C | |
| HETATM | 3681 | C19 | PC2 | | 577 | 111.123 | 30.137 | 13.434 | 1.00 | 46.58 | C | |
| HETATM | 3682 | C20 | PC2 | | 577 | 110.150 | 30.714 | 14.459 | 1.00 | 47.03 | C | |
| HETATM | 3683 | C21 | PC2 | | 577 | 109.035 | 29.767 | 14.797 | 1.00 | 48.76 | C | |
| HETATM | 3684 | C22 | PC2 | | 577 | 107.689 | 30.368 | 14.417 | 1.00 | 50.04 | C | |
| HETATM | 3685 | C23 | PC2 | | 577 | 107.108 | 31.182 | 15.547 | 1.00 | 49.67 | C | |
| HETATM | 3686 | C24 | PC2 | | 577 | 105.810 | 30.574 | 16.064 | 1.00 | 48.89 | C | |
| HETATM | 3687 | C25 | PC2 | | 577 | 104.734 | 31.630 | 16.244 | 1.00 | 50.26 | C | |
| HETATM | 3688 | C26 | PC2 | | 577 | 103.447 | 30.995 | 16.691 | 1.00 | 49.29 | C | |
| HETATM | 3689 | C27 | PC2 | | 577 | 102.382 | 32.024 | 16.975 | 1.00 | 51.78 | C | |
| HETATM | 3690 | C28 | PC2 | | 577 | 101.409 | 32.282 | 15.810 | 1.00 | 54.01 | C | |
| HETATM | 3691 | C31 | PC2 | | 577 | 122.265 | 29.713 | 18.256 | 1.00 | 56.47 | C | |
| HETATM | 3692 | C32 | PC2 | | 577 | 122.719 | 30.430 | 16.985 | 1.00 | 56.20 | C | |
| HETATM | 3693 | C33 | PC2 | | 577 | 123.655 | 29.529 | 16.141 | 1.00 | 53.24 | C | |
| HETATM | 3694 | C34 | PC2 | | 577 | 123.082 | 29.292 | 14.754 | 1.00 | 52.77 | C | |
| HETATM | 3695 | C35 | PC2 | | 577 | 122.913 | 27.816 | 14.408 | 1.00 | 49.23 | C | |
| HETATM | 3696 | C36 | PC2 | | 577 | 122.541 | 27.627 | 12.919 | 1.00 | 46.06 | C | |
| HETATM | 3697 | C37 | PC2 | | 577 | 121.192 | 28.221 | 12.542 | 1.00 | 45.16 | C | |
| HETATM | 3698 | C38 | PC2 | | 577 | 120.055 | 27.326 | 12.945 | 1.00 | 45.26 | C | |
| HETATM | 3699 | C39 | PC2 | | 577 | 118.752 | 27.801 | 12.328 | 1.00 | 47.84 | C | |
| HETATM | 3700 | C40 | PC2 | | 577 | 117.543 | 27.464 | 13.200 | 1.00 | 46.88 | C | |
| HETATM | 3701 | C41 | PC2 | | 577 | 116.897 | 26.131 | 12.833 | 1.00 | 47.97 | C | |
| HETATM | 3702 | C42 | PC2 | | 577 | 115.389 | 26.197 | 12.994 | 1.00 | 47.83 | C | |
| HETATM | 3703 | C43 | PC2 | | 577 | 114.873 | 25.007 | 13.766 | 1.00 | 48.72 | C | |
| HETATM | 3704 | C44 | PC2 | | 577 | 113.548 | 25.320 | 14.416 | 1.00 | 48.84 | C | |
| HETATM | 3705 | C45 | PC2 | | 577 | 112.964 | 24.126 | 15.143 | 1.00 | 47.68 | C | |
| HETATM | 3706 | C46 | PC2 | | 577 | 111.467 | 24.303 | 15.304 | 1.00 | 48.42 | C | |
| HETATM | 3707 | C47 | PC2 | | 577 | 110.972 | 23.732 | 16.607 | 1.00 | 48.20 | C | |
| HETATM | 3708 | C48 | PC2 | | 577 | 109.552 | 24.190 | 16.938 | 1.00 | 49.06 | C | |
| HETATM | 3709 | N | PC2 | | 577 | 120.725 | 34.725 | 25.400 | 1.00 | 60.84 | N | |
| HETATM | 3710 | O2 | PC2 | | 577 | 121.109 | 30.228 | 19.074 | 1.00 | 57.05 | O | |
| HETATM | 3711 | O3 | PC2 | | 577 | 118.448 | 32.480 | 17.912 | 1.00 | 58.04 | O | |
| HETATM | 3712 | O11 | PC2 | | 577 | 116.229 | 33.216 | 17.411 | 1.00 | 61.98 | O | |
| HETATM | 3713 | O31 | PC2 | | 577 | 122.842 | 28.650 | 18.589 | 1.00 | 58.09 | O | |
| HETATM | 3714 | O1P | PC2 | | 577 | 123.027 | 34.657 | 20.963 | 1.00 | 64.51 | O | |
| HETATM | 3715 | O2P | PC2 | | 577 | 120.836 | 35.518 | 20.050 | 1.00 | 62.32 | O | |
| HETATM | 3716 | O3P | PC2 | | 577 | 121.125 | 33.001 | 20.535 | 1.00 | 62.24 | O | |
| HETATM | 3717 | O4P | PC2 | | 577 | 121.011 | 34.654 | 22.447 | 1.00 | 63.16 | O | |
| HETATM | 3718 | P | PC2 | | 577 | 121.502 | 34.465 | 20.981 | 1.00 | 64.80 | P | |
| HETATM | 3719 | C1 | PC2 | | 578 | 86.994 | 34.830 | 31.930 | 1.00 | 61.62 | C | |
| HETATM | 3720 | C2 | PC2 | | 578 | 85.841 | 34.965 | 30.901 | 1.00 | 62.02 | C | |
| HETATM | 3721 | C3 | PC2 | | 578 | 85.868 | 33.862 | 29.802 | 1.00 | 62.35 | C | |
| HETATM | 3722 | C11 | PC2 | | 578 | 85.960 | 33.700 | 27.285 | 1.00 | 60.88 | C | |
| HETATM | 3723 | C12 | PC2 | | 578 | 85.311 | 34.735 | 26.339 | 1.00 | 59.32 | C | |
| HETATM | 3724 | C13 | PC2 | | 578 | 84.043 | 35.415 | 26.818 | 1.00 | 58.10 | C | |
| HETATM | 3725 | C14 | PC2 | | 578 | 82.918 | 34.462 | 27.127 | 1.00 | 56.18 | C | |
| HETATM | 3726 | C15 | PC2 | | 578 | 82.345 | 33.863 | 25.860 | 1.00 | 54.56 | C | |
| HETATM | 3727 | C16 | PC2 | | 578 | 82.790 | 32.421 | 25.739 | 1.00 | 52.22 | C | |
| HETATM | 3728 | C17 | PC2 | | 578 | 83.408 | 32.152 | 24.392 | 1.00 | 52.98 | C | |
| HETATM | 3729 | C18 | PC2 | | 578 | 83.871 | 30.717 | 24.277 | 1.00 | 51.10 | C | |
| HETATM | 3730 | C19 | PC2 | | 578 | 84.448 | 30.482 | 22.917 | 1.00 | 50.62 | C | |
| HETATM | 3731 | C20 | PC2 | | 578 | 85.944 | 30.570 | 22.896 | 1.00 | 49.23 | C | |
| HETATM | 3732 | C21 | PC2 | | 578 | 86.382 | 31.222 | 21.621 | 1.00 | 48.86 | C | |
| HETATM | 3733 | C22 | PC2 | | 578 | 87.865 | 31.308 | 21.562 | 1.00 | 51.74 | C | |
| HETATM | 3734 | C23 | PC2 | | 578 | 88.343 | 32.690 | 21.928 | 1.00 | 52.09 | C | |
| HETATM | 3735 | C24 | PC2 | | 578 | 89.645 | 33.031 | 21.201 | 1.00 | 53.34 | C | |
| HETATM | 3736 | C25 | PC2 | | 578 | 89.651 | 34.467 | 20.657 | 1.00 | 53.56 | C | |
| HETATM | 3737 | C26 | PC2 | | 578 | 89.281 | 34.477 | 19.186 | 1.00 | 53.09 | C | |
| HETATM | 3738 | C27 | PC2 | | 578 | 87.778 | 34.270 | 18.955 | 1.00 | 55.53 | C | |
| HETATM | 3739 | C28 | PC2 | | 578 | 87.401 | 32.905 | 18.347 | 1.00 | 54.69 | C | |
| HETATM | 3740 | C31 | PC2 | | 578 | 83.872 | 36.202 | 31.806 | 1.00 | 59.73 | C | |
| HETATM | 3741 | C32 | PC2 | | 578 | 82.863 | 36.604 | 30.692 | 1.00 | 56.28 | C | |
| HETATM | 3742 | C33 | PC2 | | 578 | 81.386 | 36.617 | 31.079 | 1.00 | 51.27 | C | |
| HETATM | 3743 | C34 | PC2 | | 578 | 80.520 | 36.146 | 29.937 | 1.00 | 47.45 | C | |
| HETATM | 3744 | C35 | PC2 | | 578 | 79.091 | 36.663 | 30.071 | 1.00 | 44.75 | C | |
| HETATM | 3745 | C36 | PC2 | | 578 | 78.132 | 35.627 | 30.661 | 1.00 | 42.16 | C | |
| HETATM | 3746 | C37 | PC2 | | 578 | 77.138 | 35.053 | 29.644 | 1.00 | 44.01 | C | |
| HETATM | 3747 | C38 | PC2 | | 578 | 77.700 | 33.789 | 29.025 | 1.00 | 48.55 | C | |
| HETATM | 3748 | C39 | PC2 | | 578 | 76.779 | 33.141 | 28.005 | 1.00 | 48.71 | C | |
| HETATM | 3749 | C40 | PC2 | | 578 | 77.578 | 32.230 | 27.052 | 1.00 | 50.41 | C | |
| HETATM | 3750 | C41 | PC2 | | 578 | 77.628 | 32.766 | 25.626 | 1.00 | 50.35 | C | |
| HETATM | 3751 | C42 | PC2 | | 578 | 78.631 | 33.930 | 25.450 | 1.00 | 50.71 | C | |
| HETATM | 3752 | C43 | PC2 | | 578 | 77.938 | 35.323 | 25.440 | 1.00 | 52.01 | C | |
| HETATM | 3753 | C44 | PC2 | | 578 | 77.116 | 35.594 | 24.178 | 1.00 | 48.11 | C | |
| HETATM | 3754 | C45 | PC2 | | 578 | 75.625 | 35.644 | 24.489 | 1.00 | 46.52 | C | |
| HETATM | 3755 | C46 | PC2 | | 578 | 74.771 | 35.884 | 23.241 | 1.00 | 48.58 | C | |
| HETATM | 3756 | C47 | PC2 | | 578 | 73.272 | 35.766 | 23.543 | 1.00 | 45.01 | C | |
| HETATM | 3757 | C48 | PC2 | | 578 | 72.569 | 34.523 | 22.985 | 1.00 | 44.41 | C | |
| HETATM | 3758 | O2 | PC2 | | 578 | 84.555 | 34.890 | 31.620 | 1.00 | 61.50 | O | |
| HETATM | 3759 | O3 | PC2 | | 578 | 86.546 | 34.266 | 28.542 | 1.00 | 61.85 | O | |
| HETATM | 3760 | O11 | PC2 | | 578 | 86.007 | 32.473 | 26.979 | 1.00 | 61.43 | O | |
| HETATM | 3761 | O31 | PC2 | | 578 | 84.118 | 36.937 | 32.806 | 1.00 | 62.13 | O | |
| HETATM | 3762 | O | HOH | | 600 | 84.165 | 23.668 | 11.967 | 1.00 | 21.08 | O | |
| HETATM | 3763 | O | HOH | | 601 | 118.125 | 29.791 | 3.819 | 1.00 | 27.75 | O | |
| HETATM | 3764 | O | HOH | | 602 | 70.149 | 31.105 | 34.270 | 1.00 | 18.49 | O | |
| HETATM | 3765 | O | HOH | | 603 | 79.512 | 28.647 | 33.051 | 1.00 | 18.96 | O | |
| HETATM | 3766 | O | HOH | | 604 | 70.538 | 30.942 | 39.043 | 1.00 | 18.86 | O | |
| HETATM | 3767 | O | HOH | | 605 | 119.573 | 16.746 | 8.921 | 1.00 | 27.63 | O | |
| HETATM | 3768 | O | HOH | | 606 | 107.282 | 22.980 | 23.340 | 1.00 | 20.11 | O | |
| HETATM | 3769 | O | HOH | | 607 | 109.393 | 24.187 | 24.821 | 1.00 | 23.06 | O | |
| HETATM | 3770 | O | HOH | | 608 | 108.438 | 26.949 | 1.654 | 1.00 | 22.26 | O | |
| HETATM | 3771 | O | HOH | | 609 | 150.557 | 34.508 | 0.708 | 1.00 | 23.00 | O | |
| HETATM | 3772 | O | HOH | | 610 | 73.597 | 33.322 | 36.572 | 1.00 | 24.35 | O | |
| HETATM | 3773 | O | HOH | | 611 | 115.493 | 23.475 | 1.026 | 1.00 | 17.94 | O | |
| HETATM | 3774 | O | HOH | | 612 | 153.597 | 40.785 | 1.406 | 1.00 | 22.50 | O | |
| HETATM | 3775 | O | HOH | | 613 | 151.671 | 22.751 | 7.992 | 1.00 | 29.22 | O | |
| HETATM | 3776 | O | HOH | | 614 | 101.357 | 27.242 | 33.267 | 1.00 | 39.25 | O | |
| HETATM | 3777 | O | HOH | | 615 | 75.759 | 42.035 | 18.093 | 1.00 | 25.98 | O | |
| HETATM | 3778 | O | HOH | | 616 | 69.042 | 30.299 | 36.834 | 1.00 | 22.54 | O | |
| HETATM | 3779 | O | HOH | | 617 | 66.882 | 39.968 | 18.354 | 1.00 | 24.93 | O | |
| HETATM | 3780 | O | HOH | | 618 | 124.577 | 13.543 | 9.803 | 1.00 | 38.85 | O | |
| HETATM | 3781 | O | HOH | | 619 | 58.117 | 29.117 | 23.652 | 1.00 | 26.70 | O | |
| HETATM | 3782 | O | HOH | | 620 | 122.240 | 24.322 | 3.407 | 1.00 | 27.36 | O | |
| HETATM | 3783 | O | HOH | | 621 | 151.729 | 23.761 | 4.689 | 1.00 | 27.10 | O | |
| HETATM | 3784 | O | HOH | | 622 | 123.381 | 37.486 | 9.617 | 1.00 | 34.51 | O | |
| HETATM | 3785 | O | HOH | | 623 | 105.192 | 24.618 | 22.288 | 1.00 | 20.50 | O | |
| HETATM | 3786 | O | HOH | | 624 | 112.577 | 17.031 | 10.950 | 1.00 | 23.13 | O | |
| HETATM | 3787 | O | HOH | | 625 | 68.255 | 38.543 | 47.337 | 1.00 | 28.60 | O | |
| HETATM | 3788 | O | HOH | | 626 | 141.576 | 23.506 | 4.319 | 1.00 | 30.21 | O | |
| HETATM | 3789 | O | HOH | | 627 | 94.885 | 18.212 | 12.932 | 1.00 | 29.78 | O | |
| HETATM | 3790 | O | HOH | | 628 | 61.737 | 32.251 | 51.574 | 1.00 | 23.90 | O | |
| HETATM | 3791 | O | HOH | | 629 | 105.807 | 18.370 | 15.601 | 1.00 | 22.56 | O | |
| HETATM | 3792 | O | HOH | | 630 | 112.993 | 35.474 | 4.344 | 1.00 | 29.17 | O | |
| HETATM | 3793 | O | HOH | | 631 | 113.576 | 34.548 | 1.924 | 1.00 | 29.34 | O | |
| HETATM | 3794 | O | HOH | | 632 | 67.986 | 42.712 | 43.674 | 1.00 | 33.32 | O | |
| HETATM | 3795 | O | HOH | | 633 | 88.011 | 39.143 | 33.387 | 1.00 | 49.94 | O | |
| HETATM | 3796 | O | HOH | | 634 | 148.292 | 33.436 | 17.793 | 1.00 | 30.67 | O | |
| HETATM | 3797 | O | HOH | | 635 | 58.382 | 42.073 | 23.521 | 1.00 | 28.36 | O | |
| HETATM | 3798 | O | HOH | | 636 | 114.984 | 27.282 | 27.222 | 1.00 | 27.16 | O | |
| HETATM | 3799 | O | HOH | | 637 | 156.456 | 40.662 | 2.248 | 1.00 | 25.44 | O | |
| HETATM | 3800 | O | HOH | | 638 | 90.154 | 46.369 | 22.690 | 1.00 | 29.16 | O | |
| HETATM | 3801 | O | HOH | | 639 | 62.250 | 34.585 | 19.684 | 1.00 | 25.07 | O | |
| HETATM | 3802 | O | HOH | | 640 | 72.453 | 27.097 | 33.204 | 1.00 | 31.42 | O | |
| HETATM | 3803 | O | HOH | | 641 | 114.904 | 15.705 | 12.003 | 1.00 | 21.58 | O | |
| HETATM | 3804 | O | HOH | | 642 | 123.877 | 18.903 | 19.587 | 1.00 | 29.76 | O | |
| HETATM | 3805 | O | HOH | | 643 | 156.476 | 34.609 | −0.986 | 1.00 | 24.32 | O | |
| HETATM | 3806 | O | HOH | | 644 | 110.541 | 41.097 | 21.054 | 1.00 | 47.68 | O | |
| HETATM | 3807 | O | HOH | | 645 | 59.862 | 42.710 | 26.761 | 1.00 | 26.65 | O | |
| HETATM | 3808 | O | HOH | | 646 | 121.792 | 21.063 | 0.952 | 1.00 | 28.87 | O | |
| HETATM | 3809 | O | HOH | | 647 | 147.063 | 19.376 | 11.110 | 1.00 | 37.21 | O | |
| HETATM | 3810 | O | HOH | | 648 | 80.118 | 44.353 | 22.877 | 1.00 | 24.05 | O | |
| HETATM | 3811 | O | HOH | | 649 | 66.949 | 43.858 | 41.168 | 1.00 | 29.23 | O | |
| HETATM | 3812 | O | HOH | | 650 | 121.851 | 19.874 | 21.078 | 1.00 | 27.34 | O | |
| HETATM | 3813 | O | HOH | | 651 | 66.496 | 27.385 | 29.201 | 1.00 | 41.55 | O | |
| HETATM | 3814 | O | HOH | | 652 | 57.737 | 46.027 | 42.082 | 1.00 | 29.12 | O | |
| HETATM | 3815 | O | HOH | | 653 | 132.496 | 22.579 | 0.588 | 1.00 | 35.69 | O | |
| HETATM | 3816 | O | HOH | | 654 | 79.724 | 43.521 | 19.119 | 1.00 | 39.42 | O | |
| HETATM | 3817 | O | HOH | | 655 | 146.296 | 39.108 | 3.659 | 1.00 | 27.69 | O | |
| HETATM | 3818 | O | HOH | | 656 | 157.244 | 40.794 | 16.353 | 1.00 | 34.49 | O | |
| HETATM | 3819 | O | HOH | | 657 | 109.980 | 12.911 | 10.044 | 1.00 | 48.41 | O | |
| HETATM | 3820 | O | HOH | | 658 | 76.988 | 28.330 | 33.249 | 1.00 | 33.22 | O | |
| HETATM | 3821 | O | HOH | | 659 | 115.513 | 24.743 | 27.838 | 1.00 | 48.49 | O | |
| HETATM | 3822 | O | HOH | | 660 | 152.446 | 19.148 | 4.996 | 1.00 | 45.90 | O | |
| HETATM | 3823 | O | HOH | | 661 | 119.882 | 31.345 | −0.439 | 1.00 | 31.38 | O | |
| HETATM | 3824 | O | HOH | | 662 | 63.940 | 29.426 | 43.558 | 1.00 | 27.44 | O | |
| HETATM | 3825 | O | HOH | | 663 | 108.569 | 14.156 | 7.472 | 1.00 | 31.27 | O | |
| HETATM | 3826 | O | HOH | | 664 | 92.364 | 23.218 | 3.396 | 1.00 | 41.96 | O | |
| HETATM | 3827 | O | HOH | | 665 | 129.328 | 32.249 | 2.058 | 1.00 | 34.24 | O | |
| HETATM | 3828 | O | HOH | | 666 | 60.790 | 27.041 | 41.788 | 1.00 | 43.62 | O | |
| HETATM | 3829 | O | HOH | | 667 | 55.153 | 27.590 | 26.291 | 1.00 | 57.69 | O | |
| HETATM | 3830 | O | HOH | | 668 | 112.053 | 24.724 | 0.381 | 1.00 | 26.48 | O | |
| HETATM | 3831 | O | HOH | | 669 | 71.535 | 37.120 | 43.420 | 1.00 | 36.10 | O | |
| HETATM | 3832 | O | HOH | | 670 | 152.485 | 38.683 | 0.095 | 1.00 | 34.47 | O | |
| HETATM | 3833 | O | HOH | | 671 | 155.059 | 32.253 | −1.042 | 1.00 | 30.25 | O | |
| HETATM | 3834 | O | HOH | | 672 | 111.138 | 14.284 | 13.013 | 1.00 | 29.81 | O | |
| HETATM | 3835 | O | HOH | | 673 | 69.315 | 47.567 | 22.031 | 1.00 | 40.85 | O | |
| HETATM | 3836 | O | HOH | | 674 | 88.892 | 15.685 | 9.494 | 1.00 | 29.01 | O | |
| HETATM | 3837 | O | HOH | | 675 | 90.677 | 34.777 | 40.315 | 1.00 | 21.65 | O | |
| HETATM | 3838 | O | HOH | | 676 | 69.174 | 35.500 | 49.454 | 1.00 | 26.98 | O | |
| HETATM | 3839 | O | HOH | | 677 | 147.497 | 23.711 | 18.579 | 1.00 | 48.10 | O | |
| HETATM | 3840 | O | HOH | | 678 | 53.792 | 36.272 | 20.287 | 1.00 | 52.58 | O | |
| HETATM | 3841 | O | HOH | | 679 | 95.255 | 37.138 | 32.574 | 1.00 | 32.28 | O | |
| HETATM | 3842 | O | HOH | | 680 | 118.975 | 24.574 | 0.202 | 1.00 | 27.94 | O | |
| HETATM | 3843 | O | HOH | | 681 | 157.015 | 43.625 | 16.085 | 1.00 | 35.02 | O | |
| HETATM | 3844 | O | HOH | | 682 | 71.015 | 37.252 | 50.263 | 1.00 | 30.20 | O | |
| HETATM | 3845 | O | HOH | | 683 | 105.844 | 26.507 | 29.332 | 1.00 | 41.67 | O | |
| HETATM | 3846 | O | HOH | | 684 | 72.619 | 30.758 | 37.168 | 1.00 | 29.31 | O | |
| HETATM | 3847 | O | HOH | | 685 | 91.117 | 36.658 | 27.489 | 1.00 | 25.14 | O | |
| HETATM | 3848 | O | HOH | | 686 | 59.826 | 28.974 | 36.483 | 1.00 | 26.96 | O | |
| HETATM | 3849 | O | HOH | | 687 | 162.581 | 36.737 | −0.268 | 1.00 | 20.53 | O | |
| HETATM | 3850 | O | HOH | | 688 | 86.908 | 24.089 | 33.784 | 1.00 | 22.35 | O | |
| HETATM | 3851 | O | HOH | | 689 | 90.944 | 13.399 | 14.833 | 1.00 | 28.37 | O | |
| HETATM | 3852 | O | HOH | | 690 | 126.240 | 20.004 | 19.576 | 1.00 | 37.29 | O | |
| HETATM | 3853 | O | HOH | | 691 | 90.232 | 37.577 | 32.820 | 1.00 | 31.92 | O | |
| HETATM | 3854 | O | HOH | | 692 | 146.372 | 29.587 | 22.048 | 1.00 | 47.56 | O | |
| HETATM | 3855 | O | HOH | | 693 | 59.625 | 35.450 | 52.705 | 1.00 | 28.99 | O | |
| HETATM | 3856 | O | HOH | | 694 | 99.546 | 23.468 | 34.480 | 1.00 | 37.18 | O | |
| HETATM | 3857 | O | HOH | | 695 | 111.569 | 14.955 | 9.158 | 1.00 | 20.48 | O | |
| HETATM | 3858 | O | HOH | | 696 | 109.181 | 35.811 | 26.432 | 1.00 | 31.77 | O | |
| HETATM | 3859 | O | HOH | | 697 | 146.411 | 28.647 | −0.165 | 1.00 | 27.19 | O | |
| HETATM | 3860 | O | HOH | | 698 | 107.049 | 35.569 | 22.940 | 1.00 | 26.40 | O | |
| HETATM | 3861 | O | HOH | | 699 | 88.913 | 25.897 | 35.466 | 1.00 | 31.19 | O | |
| HETATM | 3862 | O | HOH | | 700 | 80.943 | 21.097 | 22.741 | 1.00 | 44.09 | O | |
| HETATM | 3863 | O | HOH | | 701 | 59.884 | 52.268 | 32.933 | 1.00 | 41.47 | O | |
| HETATM | 3864 | O | HOH | | 702 | 88.600 | 18.881 | 28.890 | 1.00 | 48.28 | O | |
| HETATM | 3865 | O | HOH | | 703 | 118.459 | 16.809 | 23.165 | 1.00 | 35.83 | O | |
| HETATM | 3866 | O | HOH | | 704 | 75.861 | 44.759 | 19.660 | 1.00 | 49.00 | O | |
| HETATM | 3867 | O | HOH | | 705 | 148.896 | 41.404 | 17.323 | 1.00 | 39.32 | O | |
| HETATM | 3868 | O | HOH | | 706 | 57.705 | 51.373 | 37.515 | 1.00 | 42.86 | O | |
| HETATM | 3869 | O | HOH | | 707 | 90.470 | 20.849 | 31.454 | 1.00 | 33.79 | O | |
| HETATM | 3870 | O | HOH | | 708 | 98.710 | 27.300 | 1.950 | 1.00 | 57.96 | O | |
| HETATM | 3871 | O | HOH | | 709 | 114.909 | 13.698 | 20.364 | 1.00 | 36.36 | O | |
| HETATM | 3872 | O | HOH | | 710 | 128.227 | 18.122 | 7.681 | 1.00 | 41.51 | O | |
| HETATM | 3873 | O | HOH | | 711 | 84.343 | 40.331 | 30.987 | 1.00 | 31.65 | O | |
| HETATM | 3874 | O | HOH | | 712 | 64.870 | 28.178 | 41.483 | 1.00 | 35.92 | O | |
| HETATM | 3875 | O | HOH | | 713 | 54.115 | 30.045 | 25.716 | 1.00 | 37.12 | O | |
| HETATM | 3876 | O | HOH | | 714 | 170.800 | 30.895 | 13.617 | 1.00 | 44.37 | O | |
| HETATM | 3877 | O | HOH | | 715 | 135.759 | 19.480 | 8.584 | 1.00 | 38.78 | O | |
| HETATM | 3878 | O | HOH | | 716 | 61.416 | 54.337 | 33.040 | 1.00 | 40.11 | O | |
| HETATM | 3879 | O | HOH | | 717 | 70.513 | 31.233 | 14.897 | 1.00 | 36.70 | O | |
| HETATM | 3880 | O | HOH | | 718 | 59.429 | 37.749 | 19.202 | 1.00 | 29.70 | O | |
| HETATM | 3881 | O | HOH | | 719 | 69.018 | 43.576 | 20.186 | 1.00 | 37.93 | O | |
| HETATM | 3882 | O | HOH | | 720 | 152.683 | 44.439 | 7.806 | 1.00 | 23.28 | O | |
| HETATM | 3883 | O | HOH | | 721 | 69.215 | 26.261 | 21.940 | 1.00 | 33.59 | O | |
| HETATM | 3884 | O | HOH | | 722 | 98.704 | 44.877 | 23.423 | 1.00 | 44.80 | O | |
| HETATM | 3885 | O | HOH | | 723 | 87.846 | 36.232 | 11.402 | 1.00 | 39.79 | O | |
| HETATM | 3886 | O | HOH | | 724 | 76.298 | 28.512 | 25.264 | 1.00 | 39.88 | O | |
| HETATM | 3887 | O | HOH | | 725 | 65.656 | 27.131 | 23.451 | 1.00 | 40.38 | O | |
| HETATM | 3888 | O | HOH | | 726 | 76.954 | 38.718 | 37.760 | 1.00 | 29.18 | O | |
| HETATM | 3889 | O | HOH | | 727 | 72.127 | 44.464 | 18.845 | 1.00 | 45.57 | O | |
| HETATM | 3890 | O | HOH | | 728 | 121.819 | 18.076 | 23.415 | 1.00 | 40.29 | O | |
| HETATM | 3891 | O | HOH | | 729 | 136.251 | 34.502 | 0.126 | 1.00 | 38.20 | O | |
| HETATM | 3892 | O | HOH | | 730 | 90.079 | 20.313 | 27.612 | 1.00 | 32.09 | O | |
| HETATM | 3893 | O | HOH | | 731 | 80.930 | 22.158 | 9.764 | 1.00 | 49.58 | O | |
| HETATM | 3894 | O | HOH | | 732 | 67.095 | 47.116 | 20.879 | 1.00 | 49.75 | O | |
| HETATM | 3895 | O | HOH | | 733 | 154.005 | 47.582 | 16.984 | 1.00 | 47.08 | O | |
| HETATM | 3896 | O | HOH | | 734 | 93.497 | 43.759 | 22.460 | 1.00 | 36.22 | O | |
| HETATM | 3897 | O | HOH | | 735 | 123.975 | 16.028 | 20.691 | 1.00 | 41.90 | O | |
| HETATM | 3898 | O | HOH | | 736 | 138.143 | 19.692 | 6.394 | 1.00 | 36.56 | O | |
| HETATM | 3899 | O | HOH | | 737 | 69.252 | 28.062 | 24.561 | 1.00 | 35.33 | O | |
| HETATM | 3900 | O | HOH | | 738 | 88.975 | 40.951 | 16.566 | 1.00 | 30.91 | O | |
| HETATM | 3901 | O | HOH | | 739 | 82.594 | 24.019 | 23.496 | 1.00 | 28.76 | O | |
| HETATM | 3902 | O | HOH | | 740 | 142.679 | 28.765 | 14.461 | 1.00 | 37.71 | O | |
| HETATM | 3903 | O | HOH | | 741 | 163.315 | 38.750 | 16.038 | 1.00 | 32.52 | O | |
| HETATM | 3904 | O | HOH | | 742 | 140.477 | 20.049 | 10.607 | 1.00 | 39.02 | O | |
| HETATM | 3905 | O | HOH | | 743 | 63.822 | 26.626 | 38.993 | 1.00 | 46.07 | O | |
| HETATM | 3906 | O | HOH | | 744 | 155.846 | 47.012 | 13.006 | 1.00 | 39.84 | O | |
| HETATM | 3907 | O | HOH | | 745 | 102.339 | 21.432 | −1.541 | 1.00 | 65.06 | O | |
| HETATM | 3908 | O | HOH | | 746 | 131.504 | 40.364 | 15.835 | 1.00 | 59.13 | O | |
| HETATM | 3909 | O | HOH | | 747 | 132.517 | 38.688 | 19.028 | 1.00 | 36.09 | O | |
| HETATM | 3910 | O | HOH | | 748 | 140.272 | 36.157 | 17.398 | 1.00 | 32.62 | O | |
| HETATM | 3911 | O | HOH | | 749 | 145.027 | 27.734 | 13.192 | 1.00 | 30.23 | O | |
| HETATM | 3912 | O | HOH | | 750 | 165.145 | 39.393 | 4.138 | 1.00 | 30.48 | O | |
| HETATM | 3913 | O | HOH | | 751 | 149.283 | 29.832 | 21.072 | 1.00 | 34.58 | O | |
| HETATM | 3914 | O | HOH | | 752 | 105.122 | 16.944 | 13.211 | 1.00 | 38.22 | O | |
| HETATM | 3915 | O | HOH | | 753 | 99.521 | 42.459 | 10.102 | 1.00 | 35.81 | O | |
| HETATM | 3916 | O | HOH | | 754 | 100.450 | 40.949 | 13.986 | 1.00 | 35.66 | O | |
| HETATM | 3917 | O | HOH | | 755 | 108.933 | 39.473 | 22.726 | 1.00 | 44.20 | O | |
| HETATM | 3918 | O | HOH | | 756 | 96.199 | 19.266 | 28.127 | 1.00 | 34.23 | O | |
| HETATM | 3919 | O | HOH | | 757 | 98.861 | 20.013 | 4.596 | 1.00 | 36.98 | O | |
| HETATM | 3920 | O | HOH | | 758 | 92.369 | 35.715 | 36.473 | 1.00 | 22.87 | O | |
| HETATM | 3921 | O | HOH | | 759 | 84.124 | 46.243 | 16.859 | 1.00 | 46.81 | O | |
| HETATM | 3922 | O | HOH | | 760 | 108.204 | 40.616 | 5.066 | 1.00 | 35.36 | O | |
| HETATM | 3923 | O | HOH | | 761 | 93.508 | 15.929 | 21.132 | 1.00 | 36.61 | O | |
| HETATM | 3924 | O | HOH | | 762 | 81.980 | 35.320 | 35.482 | 1.00 | 36.35 | O | |
| HETATM | 3925 | O | HOH | | 763 | 140.231 | 31.504 | −2.668 | 1.00 | 45.36 | O | |
| HETATM | 3926 | O | HOH | | 764 | 103.511 | 41.517 | 18.364 | 1.00 | 41.85 | O | |
| HETATM | 3927 | O | HOH | | 765 | 94.866 | 20.227 | 34.149 | 1.00 | 52.60 | O | |
| HETATM | 3928 | O | HOH | | 766 | 92.144 | 19.942 | 5.830 | 1.00 | 30.00 | O | |
| HETATM | 3929 | O | HOH | | 767 | 92.493 | 44.765 | 20.132 | 1.00 | 33.65 | O | |
| HETATM | 3930 | O | HOH | | 768 | 75.829 | 25.024 | 23.495 | 1.00 | 37.74 | O | |
| HETATM | 3931 | O | HOH | | 769 | 106.630 | 13.659 | 12.956 | 1.00 | 28.74 | O | |
| HETATM | 3932 | O | HOH | | 770 | 151.375 | 35.339 | 21.807 | 1.00 | 44.52 | O | |
| HETATM | 3933 | O | HOH | | 771 | 135.682 | 25.759 | 0.758 | 1.00 | 29.89 | O | |
| HETATM | 3934 | O | HOH | | 772 | 152.829 | 21.578 | 5.856 | 1.00 | 36.44 | O | |
| HETATM | 3935 | O | HOH | | 773 | 57.464 | 41.464 | 21.070 | 1.00 | 43.85 | O | |
| HETATM | 3936 | O | HOH | | 774 | 139.894 | 22.673 | 3.437 | 1.00 | 28.84 | O | |
| HETATM | 3937 | O | HOH | | 775 | 152.226 | 46.921 | 8.720 | 1.00 | 41.21 | O | |
| HETATM | 3938 | O | HOH | | 776 | 109.851 | 26.344 | −0.818 | 1.00 | 25.73 | O | |
| HETATM | 3939 | O | HOH | | 777 | 165.149 | 44.456 | 12.143 | 1.00 | 38.51 | O | |
| HETATM | 3940 | O | HOH | | 778 | 102.435 | 18.845 | 26.427 | 1.00 | 43.28 | O | |
| HETATM | 3941 | O | HOH | | 779 | 100.110 | 39.229 | 16.764 | 1.00 | 31.02 | O | |
| HETATM | 3942 | O | HOH | | 780 | 96.129 | 17.751 | 10.589 | 1.00 | 32.67 | O | |
| HETATM | 3943 | O | HOH | | 781 | 131.014 | 22.477 | 21.435 | 1.00 | 42.25 | O | |
| HETATM | 3944 | O | HOH | | 782 | 120.604 | 15.448 | 10.801 | 1.00 | 41.74 | O | |
| HETATM | 3945 | O | HOH | | 783 | 163.466 | 43.135 | 15.379 | 1.00 | 46.48 | O | |
| HETATM | 3946 | O | HOH | | 784 | 155.317 | 45.308 | 14.992 | 1.00 | 37.63 | O | |
| HETATM | 3947 | O | HOH | | 785 | 150.231 | 43.678 | 7.025 | 1.00 | 46.11 | O | |
| HETATM | 3948 | O | HOH | | 786 | 135.972 | 20.072 | 15.547 | 1.00 | 34.19 | O | |
| HETATM | 3949 | O | HOH | | 787 | 85.518 | 16.013 | 28.791 | 1.00 | 34.72 | O | |
| HETATM | 3950 | O | HOH | | 788 | 174.108 | 37.912 | 12.316 | 1.00 | 24.85 | O | |
| HETATM | 3951 | O | HOH | | 789 | 61.092 | 29.173 | 43.272 | 1.00 | 31.17 | O | |
| HETATM | 3952 | O | HOH | | 790 | 94.569 | 28.608 | 34.977 | 1.00 | 29.27 | O | |
| HETATM | 3953 | O | HOH | | 791 | 74.231 | 21.973 | 32.900 | 1.00 | 50.90 | O | |
| HETATM | 3954 | O | HOH | | 792 | 163.104 | 35.043 | 16.441 | 1.00 | 49.62 | O | |
| HETATM | 3955 | O | HOH | | 793 | 88.902 | 18.902 | 8.063 | 1.00 | 29.96 | O | |
| HETATM | 3956 | O | HOH | | 794 | 151.103 | 37.446 | 24.513 | 1.00 | 51.18 | O | |
| HETATM | 3957 | O | HOH | | 795 | 70.942 | 40.577 | 18.429 | 1.00 | 39.55 | O | |
| HETATM | 3958 | O | HOH | | 796 | 117.220 | 31.093 | 25.182 | 1.00 | 35.18 | O | |
| HETATM | 3959 | O | HOH | | 797 | 110.206 | 37.559 | 24.758 | 1.00 | 32.16 | O | |
| HETATM | 3960 | O | HOH | | 798 | 96.225 | 15.839 | 18.808 | 1.00 | 36.05 | O | |
| HETATM | 3961 | O | HOH | | 799 | 113.513 | 13.535 | 12.743 | 1.00 | 43.83 | O | |
| HETATM | 3962 | O | HOH | | 800 | 119.461 | 32.010 | 2.490 | 1.00 | 30.34 | O | |
| HETATM | 3963 | O | HOH | | 801 | 162.424 | 41.337 | 17.354 | 1.00 | 36.07 | O | |
| HETATM | 3964 | O | HOH | | 802 | 130.761 | 22.385 | 2.692 | 1.00 | 42.71 | O | |
| HETATM | 3965 | O | HOH | | 803 | 121.910 | 13.054 | 8.759 | 1.00 | 48.96 | O | |
| HETATM | 3966 | O | HOH | | 804 | 104.355 | 17.000 | 7.578 | 1.00 | 40.59 | O | |
| HETATM | 3967 | O | HOH | | 805 | 148.592 | 43.686 | 10.498 | 1.00 | 38.17 | O | |
| HETATM | 3968 | O | HOH | | 806 | 118.786 | 33.859 | 4.580 | 1.00 | 33.89 | O | |
| HETATM | 3969 | O | HOH | | 807 | 121.315 | 36.698 | 16.426 | 1.00 | 30.64 | O | |
| HETATM | 3970 | O | HOH | | 808 | 130.070 | 28.160 | 20.674 | 1.00 | 35.12 | O | |
| HETATM | 3971 | O | HOH | | 809 | 130.545 | 37.782 | 21.836 | 1.00 | 45.09 | O | |
| HETATM | 3972 | O | HOH | | 810 | 139.410 | 29.130 | 20.308 | 1.00 | 51.26 | O | |
| HETATM | 3973 | O | HOH | | 811 | 150.228 | 24.531 | 19.243 | 1.00 | 36.32 | O | |
| HETATM | 3974 | O | HOH | | 812 | 154.418 | 26.491 | 17.387 | 1.00 | 33.90 | O | |
| HETATM | 3975 | O | HOH | | 813 | 155.138 | 29.214 | 19.843 | 1.00 | 44.59 | O | |
| HETATM | 3976 | O | HOH | | 814 | 161.192 | 28.681 | 16.293 | 1.00 | 37.97 | O | |
| HETATM | 3977 | O | HOH | | 815 | 167.542 | 31.317 | 15.840 | 1.00 | 47.80 | O | |
| HETATM | 3978 | O | HOH | | 816 | 125.138 | 28.083 | 1.710 | 1.00 | 38.13 | O | |
| HETATM | 3979 | O | HOH | | 817 | 115.719 | 13.005 | 1.788 | 1.00 | 47.10 | O | |
| HETATM | 3980 | O | HOH | | 818 | 121.231 | 17.065 | 6.662 | 1.00 | 44.28 | O | |
| HETATM | 3981 | O | HOH | | 819 | 148.456 | 44.805 | 15.415 | 1.00 | 52.81 | O | |
| HETATM | 3982 | O | HOH | | 820 | 165.597 | 32.633 | 17.489 | 1.00 | 43.14 | O | |
| HETATM | 3983 | O | HOH | | 821 | 162.652 | 44.400 | 11.679 | 1.00 | 41.81 | O | |
| HETATM | 3984 | O | HOH | | 822 | 156.092 | 44.470 | 8.805 | 1.00 | 23.14 | O | |
| HETATM | 3985 | O | HOH | | 823 | 151.412 | 34.978 | −1.177 | 1.00 | 51.79 | O | |
| HETATM | 3986 | O | HOH | | 824 | 146.291 | 31.250 | −3.811 | 1.00 | 37.67 | O | |
| HETATM | 3987 | O | HOH | | 825 | 146.652 | 21.446 | 12.953 | 1.00 | 29.77 | O | |
| HETATM | 3988 | O | HOH | | 826 | 150.914 | 21.632 | 14.330 | 1.00 | 60.29 | O | |
| HETATM | 3989 | O | HOH | | 827 | 147.767 | 24.264 | 15.647 | 1.00 | 37.90 | O | |
| HETATM | 3990 | O | HOH | | 828 | 118.239 | 22.197 | 26.336 | 1.00 | 37.95 | O | |
| HETATM | 3991 | O | HOH | | 829 | 63.716 | 30.262 | 47.058 | 1.00 | 22.34 | O | |
| HETATM | 3992 | O | HOH | | 830 | 119.913 | 38.538 | 15.478 | 1.00 | 45.30 | O | |
| HETATM | 3993 | O | HOH | | 831 | 111.319 | 39.953 | 9.439 | 1.00 | 26.28 | O | |
| HETATM | 3994 | O | HOH | | 832 | 115.475 | 42.297 | 14.232 | 1.00 | 53.11 | O | |
| HETATM | 3995 | O | HOH | | 833 | 92.734 | 26.431 | 35.997 | 1.00 | 43.59 | O | |
| HETATM | 3996 | O | HOH | | 834 | 64.798 | 54.408 | 33.323 | 1.00 | 40.01 | O | |
| HETATM | 3997 | O | HOH | | 835 | 73.057 | 42.215 | 19.412 | 1.00 | 36.23 | O | |
| HETATM | 3998 | O | HOH | | 836 | 68.772 | 41.714 | 18.186 | 1.00 | 30.98 | O | |
| HETATM | 3999 | O | HOH | | 837 | 64.284 | 46.260 | 22.870 | 1.00 | 33.08 | O | |
| HETATM | 4000 | O | HOH | | 838 | 51.819 | 46.802 | 36.972 | 1.00 | 49.30 | O | |
| HETATM | 4001 | O | HOH | | 839 | 61.815 | 30.441 | 49.451 | 1.00 | 25.71 | O | |
| HETATM | 4002 | O | HOH | | 840 | 108.833 | 30.076 | 1.473 | 1.00 | 47.10 | O | |
| HETATM | 4003 | O | HOH | | 841 | 152.525 | 24.454 | 15.434 | 1.00 | 34.70 | O | |
| HETATM | 4004 | O | HOH | | 842 | 143.739 | 42.754 | 21.023 | 1.00 | 50.03 | O | |
| HETATM | 4005 | O | HOH | | 843 | 149.171 | 43.279 | 13.651 | 1.00 | 38.57 | O | |
| HETATM | 4006 | O | HOH | | 844 | 122.510 | 34.797 | 6.476 | 1.00 | 45.60 | O | |
| HETATM | 4007 | O | HOH | | 845 | 143.681 | 40.244 | 7.035 | 1.00 | 39.12 | O | |
| HETATM | 4008 | O | HOH | | 846 | 128.609 | 16.210 | 15.979 | 1.00 | 52.82 | O | |
| HETATM | 4009 | O | HOH | | 847 | 147.972 | 31.179 | −1.305 | 1.00 | 46.95 | O | |
| HETATM | 4010 | O | HOH | | 848 | 150.699 | 40.290 | −0.741 | 1.00 | 48.63 | O | |
| HETATM | 4011 | O | HOH | | 849 | 152.288 | 47.132 | 2.636 | 1.00 | 54.66 | O | |
| HETATM | 4012 | O | HOH | | 850 | 140.836 | 20.556 | 7.851 | 1.00 | 36.11 | O | |
| HETATM | 4013 | O | HOH | | 851 | 145.790 | 17.583 | 6.027 | 1.00 | 48.50 | O | |
| HETATM | 4014 | O | HOH | | 852 | 120.951 | 21.863 | 25.632 | 1.00 | 37.97 | O | |
| HETATM | 4015 | O | HOH | | 853 | 126.465 | 35.258 | 19.933 | 1.00 | 44.79 | O | |
| HETATM | 4016 | O | HOH | | 854 | 99.050 | 26.095 | 33.949 | 1.00 | 40.92 | O | |
| HETATM | 4017 | O | HOH | | 855 | 101.578 | 40.601 | 17.498 | 1.00 | 42.01 | O | |
| HETATM | 4018 | O | HOH | | 856 | 55.840 | 31.049 | 24.084 | 1.00 | 34.43 | O | |
| HETATM | 4019 | O | HOH | | 857 | 54.576 | 31.649 | 21.882 | 1.00 | 37.29 | O | |
| HETATM | 4020 | O | HOH | | 858 | 107.147 | 38.143 | 21.777 | 1.00 | 41.29 | O | |
| HETATM | 4021 | O | HOH | | 859 | 105.900 | 14.619 | 7.169 | 1.00 | 35.18 | O | |
| HETATM | 4022 | O | HOH | | 860 | 116.770 | 16.258 | 18.155 | 1.00 | 27.05 | O | |
| HETATM | 4023 | O | HOH | | 861 | 69.225 | 34.124 | 14.708 | 1.00 | 33.72 | O | |
| HETATM | 4024 | O | HOH | | 862 | 106.229 | 19.293 | 7.105 | 1.00 | 39.37 | O | |
| HETATM | 4025 | O | HOH | | 863 | 77.224 | 43.597 | 32.842 | 1.00 | 40.57 | O | |
| HETATM | 4026 | O | HOH | | 864 | 161.617 | 45.539 | 2.273 | 1.00 | 32.00 | O | |
| HETATM | 4027 | O | HOH | | 865 | 161.920 | 30.571 | 1.633 | 1.00 | 31.06 | O | |
| HETATM | 4028 | O | HOH | | 866 | 156.327 | 24.681 | 17.370 | 1.00 | 27.75 | O | |
| HETATM | 4029 | O | HOH | | 867 | 96.184 | 36.978 | 15.196 | 1.00 | 41.87 | O | |
| HETATM | 4030 | O | HOH | | 868 | 55.480 | 25.734 | 32.879 | 1.00 | 30.85 | O | |
| HETATM | 4031 | O | HOH | | 869 | 106.580 | 32.589 | 25.649 | 1.00 | 34.75 | O | |
| HETATM | 4032 | O | HOH | | 870 | 50.312 | 40.619 | 37.293 | 1.00 | 40.30 | O | |
| HETATM | 4033 | O | HOH | | 871 | 61.888 | 42.769 | 46.652 | 1.00 | 33.46 | O | |
| HETATM | 4034 | O | HOH | | 872 | 115.833 | 18.453 | 26.468 | 1.00 | 42.41 | O | |
| HETATM | 4035 | O | HOH | | 873 | 117.285 | 19.219 | 28.375 | 1.00 | 51.17 | O | |
| HETATM | 4036 | O | HOH | | 874 | 101.801 | 26.935 | 3.329 | 1.00 | 46.93 | O | |
| HETATM | 4037 | O | HOH | | 875 | 72.830 | 28.698 | 18.685 | 1.00 | 39.82 | O | |
| HETATM | 4038 | O | HOH | | 876 | 107.025 | 20.942 | 29.856 | 1.00 | 38.35 | O | |
| HETATM | 4039 | O | HOH | | 877 | 77.248 | 45.117 | 22.622 | 1.00 | 33.37 | O | |
| HETATM | 4040 | O | HOH | | 878 | 106.575 | 20.058 | 0.784 | 1.00 | 32.74 | O | |
| HETATM | 4041 | O | HOH | | 879 | 113.147 | 13.152 | 15.219 | 1.00 | 41.45 | O | |
| HETATM | 4042 | O | HOH | | 880 | 155.196 | 24.185 | 11.650 | 1.00 | 43.65 | O | |
| HETATM | 4043 | O | HOH | | 881 | 154.331 | 25.515 | 13.624 | 1.00 | 32.54 | O | |
| HETATM | 4044 | O | HOH | | 882 | 123.346 | 38.135 | 17.229 | 1.00 | 36.23 | O | |
| HETATM | 4045 | O | HOH | | 883 | 52.761 | 28.518 | 36.319 | 1.00 | 40.91 | O | |
| HETATM | 4046 | O | HOH | | 884 | 81.340 | 34.434 | 10.456 | 1.00 | 43.97 | O | |
| HETATM | 4047 | O | HOH | | 885 | 164.641 | 46.049 | 1.631 | 1.00 | 40.97 | O | |
| HETATM | 4048 | O | HOH | | 886 | 155.437 | 21.390 | 8.538 | 1.00 | 40.35 | O | |
| HETATM | 4049 | O | HOH | | 887 | 63.917 | 41.371 | 50.043 | 1.00 | 39.24 | O | |
| HETATM | 4050 | O | HOH | | 888 | 123.710 | 32.172 | 25.832 | 1.00 | 44.56 | O | |
| HETATM | 4051 | O | HOH | | 889 | 58.715 | 49.682 | 38.988 | 1.00 | 47.00 | O | |
| HETATM | 4052 | O | HOH | | 890 | 104.230 | 14.552 | 9.231 | 1.00 | 38.00 | O | |
| HETATM | 4053 | O | HOH | | 891 | 89.121 | 24.453 | 3.218 | 1.00 | 44.48 | O | |
| HETATM | 4054 | O | HOH | | 892 | 89.963 | 22.112 | 2.715 | 1.00 | 52.95 | O | |
| HETATM | 4055 | O | HOH | | 893 | 80.309 | 34.123 | 37.159 | 1.00 | 41.56 | O | |
| HETATM | 4056 | O | HOH | | 894 | 50.820 | 42.137 | 27.517 | 1.00 | 45.59 | O | |
| HETATM | 4057 | O | HOH | | 895 | 132.400 | 22.217 | 4.400 | 1.00 | 33.87 | O | |
| HETATM | 4058 | O | HOH | | 896 | 133.073 | 19.595 | 16.021 | 1.00 | 48.75 | O | |
| HETATM | 4059 | O | HOH | | 897 | 60.299 | 42.001 | 49.556 | 1.00 | 44.94 | O | |
| HETATM | 4060 | O | HOH | | 898 | 148.453 | 22.262 | 14.649 | 1.00 | 44.77 | O | |
| HETATM | 4061 | O | HOH | | 899 | 81.576 | 24.201 | 8.323 | 1.00 | 69.20 | O | |
| HETATM | 4062 | O | HOH | | 900 | 76.494 | 26.185 | 21.200 | 1.00 | 41.15 | O | |
| HETATM | 4063 | O | HOH | | 901 | 110.529 | 33.372 | 26.395 | 1.00 | 39.74 | O | |
| HETATM | 4064 | O | HOH | | 902 | 87.373 | 43.064 | 31.908 | 1.00 | 45.67 | O | |
| HETATM | 4065 | O | HOH | | 903 | 80.822 | 19.174 | 24.998 | 1.00 | 48.54 | O | |
| HETATM | 4066 | O | HOH | | 904 | 114.486 | 16.480 | 26.313 | 1.00 | 52.76 | O | |
| HETATM | 4067 | O | HOH | | 905 | 102.668 | 16.759 | 24.664 | 1.00 | 33.84 | O | |
| HETATM | 4068 | O | HOH | | 906 | 81.395 | 22.735 | 21.353 | 1.00 | 54.24 | O | |
| HETATM | 4069 | O | HOH | | 907 | 92.266 | 12.498 | 17.010 | 1.00 | 41.25 | O | |
| HETATM | 4070 | O | HOH | | 908 | 140.566 | 22.484 | 17.874 | 1.00 | 41.09 | O | |
| HETATM | 4071 | O | HOH | | 909 | 108.747 | 26.840 | 27.788 | 1.00 | 37.32 | O | |
| HETATM | 4072 | O | HOH | | 910 | 65.582 | 29.275 | 21.957 | 1.00 | 47.66 | O | |
| HETATM | 4073 | O | HOH | | 911 | 105.924 | 14.765 | 21.331 | 1.00 | 30.45 | O | |
| HETATM | 4074 | O | HOH | | 912 | 61.805 | 38.633 | 16.917 | 1.00 | 42.23 | O | |
| HETATM | 4075 | O | HOH | | 913 | 71.330 | 51.912 | 24.092 | 1.00 | 50.84 | O | |
| HETATM | 4076 | O | HOH | | 914 | 87.612 | 39.996 | 30.692 | 1.00 | 43.06 | O | |
| HETATM | 4077 | O | HOH | | 915 | 76.320 | 38.318 | 12.947 | 1.00 | 49.95 | O | |
| HETATM | 4078 | O | HOH | | 916 | 63.606 | 26.203 | 20.193 | 1.00 | 44.91 | O | |
| HETATM | 4079 | O | HOH | | 917 | 128.927 | 24.825 | 2.715 | 1.00 | 37.40 | O | |
| HETATM | 4080 | O | HOH | | 918 | 164.440 | 24.988 | 6.034 | 1.00 | 48.18 | O | |
| HETATM | 4081 | O | HOH | | 919 | 158.919 | 46.596 | 12.205 | 1.00 | 41.72 | O | |
| HETATM | 4082 | O | HOH | | 920 | 166.343 | 44.608 | 1.107 | 1.00 | 44.66 | O | |
| HETATM | 4083 | O | HOH | | 921 | 63.772 | 28.487 | 18.826 | 1.00 | 44.56 | O | |
| HETATM | 4084 | O | HOH | | 922 | 94.239 | 13.856 | 17.494 | 1.00 | 51.75 | O | |
| HETATM | 4085 | O | HOH | | 923 | 112.642 | 13.764 | 29.772 | 1.00 | 53.93 | O | |
| HETATM | 4086 | O | HOH | | 924 | 105.528 | 26.307 | 3.412 | 1.00 | 44.89 | O | |
| HETATM | 4087 | O | HOH | | 925 | 112.574 | 25.374 | 28.494 | 1.00 | 46.08 | O | |
| HETATM | 4088 | O | HOH | | 926 | 159.002 | 47.297 | 1.994 | 1.00 | 30.69 | O | |
| HETATM | 4089 | O | HOH | | 927 | 168.738 | 25.307 | 12.173 | 1.00 | 47.73 | O | |
| HETATM | 4090 | O | HOH | | 928 | 130.802 | 15.541 | 11.998 | 1.00 | 41.37 | O | |
| HETATM | 4091 | O | HOH | | 929 | 125.657 | 30.349 | 1.536 | 1.00 | 48.67 | O | |
| HETATM | 4092 | O | HOH | | 930 | 52.878 | 40.908 | 35.137 | 1.00 | 39.90 | O | |
| HETATM | 4093 | O | HOH | | 931 | 173.404 | 34.514 | 8.786 | 1.00 | 45.88 | O | |
| HETATM | 4094 | O | HOH | | 932 | 174.038 | 32.207 | 4.516 | 1.00 | 50.98 | O | |
| HETATM | 4095 | O | HOH | | 933 | 58.948 | 23.815 | 39.014 | 1.00 | 48.58 | O | |
| HETATM | 4096 | O | HOH | | 934 | 171.141 | 25.826 | 10.192 | 1.00 | 43.72 | O | |
| HETATM | 4097 | O | HOH | | 935 | 138.816 | 17.114 | 6.239 | 1.00 | 51.73 | O | |
| HETATM | 4098 | O | HOH | | 936 | 57.703 | 30.554 | 42.247 | 1.00 | 38.78 | O | |
| HETATM | 4099 | O | HOH | | 937 | 73.186 | 26.592 | 27.737 | 1.00 | 41.12 | O | |
| HETATM | 4100 | O | HOH | | 938 | 114.786 | 41.338 | 11.605 | 1.00 | 36.01 | O | |
| HETATM | 4101 | O | HOH | | 939 | 112.711 | 11.095 | 10.160 | 1.00 | 29.76 | O | |
| HETATM | 4102 | O | HOH | | 940 | 104.813 | 24.020 | 30.984 | 1.00 | 33.95 | O | |
| HETATM | 4103 | O | HOH | | 941 | 59.405 | 35.938 | 16.859 | 1.00 | 46.75 | O | |
| HETATM | 4104 | O | HOH | | 942 | 80.586 | 44.462 | 16.730 | 1.00 | 50.32 | O | |
| HETATM | 4105 | O | HOH | | 943 | 95.242 | 17.442 | 20.288 | 1.00 | 48.12 | O | |
| HETATM | 4106 | O | HOH | | 944 | 118.329 | 13.406 | 11.659 | 1.00 | 48.74 | O | |
| HETATM | 4107 | O | HOH | | 945 | 102.783 | 25.226 | 34.776 | 1.00 | 56.86 | O | |
| HETATM | 4108 | O | HOH | | 946 | 81.548 | 48.319 | 17.358 | 1.00 | 47.92 | O | |
| HETATM | 4109 | O | HOH | | 947 | 111.457 | 39.065 | 4.726 | 1.00 | 44.11 | O | |
| HETATM | 4110 | O | HOH | | 948 | 112.739 | 39.709 | 6.931 | 1.00 | 29.09 | O | |
| HETATM | 4111 | O | HOH | | 949 | 133.431 | 40.642 | 11.124 | 1.00 | 34.53 | O | |
| HETATM | 4112 | O | HOH | | 950 | 154.877 | 31.262 | 21.008 | 1.00 | 33.91 | O | |
| HETATM | 4113 | O | HOH | | 951 | 124.157 | 30.842 | −0.320 | 1.00 | 45.25 | O | |
| HETATM | 4114 | O | HOH | | 952 | 79.250 | 24.969 | 35.208 | 1.00 | 37.33 | O | |
| HETATM | 4115 | O | HOH | | 953 | 60.260 | 26.416 | 39.304 | 1.00 | 41.17 | O | |
| HETATM | 4116 | O | HOH | | 954 | 144.358 | 21.133 | 14.031 | 1.00 | 46.73 | O | |
| HETATM | 4117 | O | HOH | | 955 | 77.925 | 35.184 | 38.555 | 1.00 | 41.95 | O | |
| HETATM | 4118 | O | HOH | | 956 | 164.916 | 23.852 | 10.192 | 1.00 | 49.14 | O | |
| HETATM | 4119 | O | HOH | | 957 | 97.622 | 19.483 | 10.877 | 1.00 | 39.78 | O | |
| HETATM | 4120 | O | HOH | | 958 | 154.791 | 45.833 | 10.794 | 1.00 | 41.16 | O | |
| HETATM | 4121 | O | HOH | | 959 | 166.086 | 30.480 | 0.903 | 1.00 | 41.69 | O | |
| HETATM | 4122 | O | HOH | | 960 | 70.539 | 26.740 | 26.849 | 1.00 | 37.97 | O | |
| HETATM | 4123 | O | HOH | | 961 | 56.103 | 43.120 | 25.916 | 1.00 | 38.96 | O | |
| HETATM | 4124 | O | HOH | | 962 | 149.861 | 27.279 | −0.455 | 1.00 | 41.71 | O | |
| HETATM | 4125 | O | HOH | | 963 | 59.435 | 29.715 | 20.995 | 1.00 | 34.53 | O | |
| HETATM | 4126 | O | HOH | | 964 | 150.227 | 26.129 | 16.990 | 1.00 | 37.54 | O | |
| HETATM | 4127 | O | HOH | | 965 | 133.292 | 17.437 | 4.000 | 1.00 | 39.92 | O | |
| HETATM | 4128 | O | HOH | | 966 | 123.896 | 14.408 | 17.440 | 1.00 | 41.00 | O | |
| HETATM | 4129 | O | HOH | | 967 | 51.062 | 34.617 | 20.368 | 1.00 | 41.20 | O | |
| HETATM | 4130 | O | HOH | | 968 | 120.380 | 37.559 | 22.871 | 1.00 | 40.73 | O | |
| HETATM | 4131 | O | HOH | | 969 | 126.208 | 22.127 | 25.561 | 1.00 | 45.32 | O | |
| HETATM | 4132 | O | HOH | | 970 | 147.061 | 23.515 | 2.663 | 1.00 | 43.25 | O | |
| HETATM | 4133 | O | HOH | | 971 | 81.184 | 24.886 | 38.284 | 1.00 | 41.16 | O | |
| HETATM | 4134 | O | HOH | | 972 | 86.924 | 21.739 | 32.071 | 1.00 | 38.90 | O | |
| HETATM | 4135 | O | HOH | | 973 | 77.139 | 47.107 | 30.409 | 1.00 | 40.17 | O | |
| HETATM | 4136 | O | HOH | | 974 | 143.226 | 34.622 | 18.413 | 1.00 | 40.67 | O | |
| HETATM | 4137 | O | HOH | | 975 | 49.420 | 41.350 | 32.003 | 1.00 | 45.02 | O | |
| HETATM | 4138 | O | HOH | | 976 | 140.806 | 40.144 | 2.737 | 1.00 | 38.01 | O | |
| HETATM | 4139 | O | HOH | | 977 | 61.977 | 26.445 | 36.528 | 1.00 | 39.65 | O | |
| HETATM | 4140 | O | HOH | | 978 | 132.596 | 28.115 | 22.444 | 1.00 | 45.78 | O | |
| HETATM | 4141 | O | HOH | | 979 | 89.594 | 21.309 | 5.594 | 1.00 | 43.15 | O | |
| HETATM | 4142 | O | HOH | | 980 | 136.533 | 27.240 | 18.859 | 1.00 | 38.68 | O | |
| HETATM | 4143 | O | HOH | | 981 | 81.636 | 45.581 | 20.509 | 1.00 | 38.28 | O | |
| HETATM | 4144 | O | HOH | | 982 | 150.176 | 23.125 | 1.828 | 1.00 | 42.74 | O | |
| HETATM | 4145 | O | HOH | | 983 | 102.467 | 17.446 | 5.500 | 1.00 | 41.73 | O | |
| HETATM | 4146 | O | HOH | | 984 | 71.891 | 54.910 | 33.932 | 1.00 | 49.74 | O | |
| HETATM | 4147 | O | HOH | | 985 | 84.869 | 35.360 | 11.358 | 1.00 | 40.38 | O | |
| HETATM | 4148 | O | HOH | | 986 | 142.653 | 36.231 | 0.185 | 1.00 | 43.33 | O | |
| CONECT | 1044 | 1375 | | | | | | | | | | |
| CONECT | 1375 | 1044 | | | | | | | | | | |
| CONECT | 3665 | 3666 | 3716 | | | | | | | | | |
| CONECT | 3666 | 3665 | 3667 | 3710 | | | | | | | | |
| CONECT | 3667 | 3666 | 3711 | | | | | | | | | |
| CONECT | 3668 | 3669 | 3717 | | | | | | | | | |
| CONECT | 3669 | 3668 | 3709 | | | | | | | | | |
| CONECT | 3670 | 3709 | | | | | | | | | | |
| CONECT | 3671 | 3709 | | | | | | | | | | |
| CONECT | 3672 | 3709 | | | | | | | | | | |
| CONECT | 3673 | 3674 | 3711 | 3712 | | | | | | | | |
| CONECT | 3674 | 3673 | 3675 | | | | | | | | | |
| CONECT | 3675 | 3674 | 3676 | | | | | | | | | |
| CONECT | 3676 | 3675 | 3677 | | | | | | | | | |
| CONECT | 3677 | 3676 | 3678 | | | | | | | | | |
| CONECT | 3678 | 3677 | 3679 | | | | | | | | | |
| CONECT | 3679 | 3678 | 3680 | | | | | | | | | |
| CONECT | 3680 | 3679 | 3681 | | | | | | | | | |
| CONECT | 3681 | 3680 | 3682 | | | | | | | | | |
| CONECT | 3682 | 3681 | 3683 | | | | | | | | | |
| CONECT | 3683 | 3682 | 3684 | | | | | | | | | |
| CONECT | 3684 | 3683 | 3685 | | | | | | | | | |
| CONECT | 3685 | 3684 | 3686 | | | | | | | | | |
| CONECT | 3686 | 3685 | 3687 | | | | | | | | | |
| CONECT | 3687 | 3686 | 3688 | | | | | | | | | |
| CONECT | 3688 | 3687 | 3689 | | | | | | | | | |
| CONECT | 3689 | 3688 | 3690 | | | | | | | | | |
| CONECT | 3690 | 3689 | | | | | | | | | | |
| CONECT | 3691 | 3692 | 3710 | 3713 | | | | | | | | |
| CONECT | 3692 | 3691 | 3693 | | | | | | | | | |
| CONECT | 3693 | 3692 | 3694 | | | | | | | | | |
| CONECT | 3694 | 3693 | 3695 | | | | | | | | | |
| CONECT | 3695 | 3694 | 3696 | | | | | | | | | |
| CONECT | 3696 | 3695 | 3697 | | | | | | | | | |
| CONECT | 3697 | 3696 | 3698 | | | | | | | | | |
| CONECT | 3698 | 3697 | 3699 | | | | | | | | | |
| CONECT | 3699 | 3698 | 3700 | | | | | | | | | |
| CONECT | 3700 | 3699 | 3701 | | | | | | | | | |
| CONECT | 3701 | 3700 | 3702 | | | | | | | | | |
| CONECT | 3702 | 3701 | 3703 | | | | | | | | | |
| CONECT | 3703 | 3702 | 3704 | | | | | | | | | |
| CONECT | 3704 | 3703 | 3705 | | | | | | | | | |
| CONECT | 3705 | 3704 | 3706 | | | | | | | | | |
| CONECT | 3706 | 3705 | 3707 | | | | | | | | | |
| CONECT | 3707 | 3706 | 3708 | | | | | | | | | |
| CONECT | 3708 | 3707 | | | | | | | | | | |
| CONECT | 3709 | 3669 | 3670 | 3671 | 3672 | | | | | | | |
| CONECT | 3710 | 3666 | 3691 | | | | | | | | | |
| CONECT | 3711 | 3667 | 3673 | | | | | | | | | |
| CONECT | 3712 | 3673 | | | | | | | | | | |
| CONECT | 3713 | 3691 | | | | | | | | | | |
| CONECT | 3714 | 3718 | | | | | | | | | | |
| CONECT | 3715 | 3718 | | | | | | | | | | |
| CONECT | 3716 | 3665 | 3718 | | | | | | | | | |
| CONECT | 3717 | 3668 | 3718 | | | | | | | | | |
| CONECT | 3718 | 3714 | 3715 | 3716 | 3717 | | | | | | | |
| CONECT | 3719 | 3720 | | | | | | | | | | |
| CONECT | 3720 | 3719 | 3721 | 3758 | | | | | | | | |
| CONECT | 3721 | 3720 | 3759 | | | | | | | | | |
| CONECT | 3722 | 3723 | 3759 | 3760 | | | | | | | | |
| CONECT | 3723 | 3722 | 3724 | | | | | | | | | |
| CONECT | 3724 | 3723 | 3725 | | | | | | | | | |
| CONECT | 3725 | 3724 | 3726 | | | | | | | | | |
| CONECT | 3726 | 3725 | 3727 | | | | | | | | | |
| CONECT | 3727 | 3726 | 3728 | | | | | | | | | |
| CONECT | 3728 | 3727 | 3729 | | | | | | | | | |
| CONECT | 3729 | 3728 | 3730 | | | | | | | | | |
| CONECT | 3730 | 3729 | 3731 | | | | | | | | | |
| CONECT | 3731 | 3730 | 3732 | | | | | | | | | |
| CONECT | 3732 | 3731 | 3733 | | | | | | | | | |
| CONECT | 3733 | 3732 | 3734 | | | | | | | | | |
| CONECT | 3734 | 3733 | 3735 | | | | | | | | | |
| CONECT | 3735 | 3734 | 3736 | | | | | | | | | |
| CONECT | 3736 | 3735 | 3737 | | | | | | | | | |
| CONECT | 3737 | 3736 | 3738 | | | | | | | | | |
| CONECT | 3738 | 3737 | 3739 | | | | | | | | | |
| CONECT | 3739 | 3738 | | | | | | | | | | |
| CONECT | 3740 | 3741 | 3758 | 3761 | | | | | | | | |
| CONECT | 3741 | 3740 | 3742 | | | | | | | | | |
| CONECT | 3742 | 3741 | 3743 | | | | | | | | | |
| CONECT | 3743 | 3742 | 3744 | | | | | | | | | |
| CONECT | 3744 | 3743 | 3745 | | | | | | | | | |
| CONECT | 3745 | 3744 | 3746 | | | | | | | | | |
| CONECT | 3746 | 3745 | 3747 | | | | | | | | | |
| CONECT | 3747 | 3746 | 3748 | | | | | | | | | |
| CONECT | 3748 | 3747 | 3749 | | | | | | | | | |
| CONECT | 3749 | 3748 | 3750 | | | | | | | | | |
| CONECT | 3750 | 3749 | 3751 | | | | | | | | | |
| CONECT | 3751 | 3750 | 3752 | | | | | | | | | |
| CONECT | 3752 | 3751 | 3753 | | | | | | | | | |
| CONECT | 3753 | 3752 | 3754 | | | | | | | | | |
| CONECT | 3754 | 3753 | 3755 | | | | | | | | | |
| CONECT | 3755 | 3754 | 3756 | | | | | | | | | |
| CONECT | 3756 | 3755 | 3757 | | | | | | | | | |
| CONECT | 3757 | 3756 | | | | | | | | | | |
| CONECT | 3758 | 3720 | 3740 | | | | | | | | | |
| CONECT | 3759 | 3721 | 3722 | | | | | | | | | |
| CONECT | 3760 | 3722 | | | | | | | | | | |
| CONECT | 3761 | 3740 | | | | | | | | | | |
| MASTER | 323 | 0 | 2 | 12 | 23 | 0 | 0 | 6 | 4147 | 1 | 99 | 36 |
| END | | | | | | | | | | | | |

| HEADER | | ANTIBIOTIC 25-APR-00 1EWF | | |
|---|---|---|---|---|
| TITLE | | THE 1.7 ANGSTROM CRYSTAL STRUCTURE OF BPI | | |
| COMPND | | MOL_ID: 1; | | |
| COMPND | 2 | MOLECULE: BACTERICIDAL/PERMEABILITY-INCREASING PROTEIN; | | |
| COMPND | 3 | CHAIN: A; | | |
| COMPND | 4 | SYNONYM: BPI; | | |
| COMPND | 5 | ENGINEERED: YES; | | |
| COMPND | 6 | MUTATION: YES | | |
| SOURCE | | MOL_ID: 1; | | |
| SOURCE | 2 | ORGANISM_SCIENTIFIC: HOMO SAPIENS; | | |
| SOURCE | 3 | ORGANISM_COMMON: HUMAN; | | |
| SOURCE | 4 | EXPRESSION_SYSTEM: CRICETULUS GRISEUS; | | |
| SOURCE | 5 | EXPRESSION_SYSTEM_COMMON: CHINESE HAMSTER; | | |
| SOURCE | 6 | EXPRESSION_SYSTEM_CELL: OVARY CELLS | | |
| KEYWDS | | BACTERICIDAL, PERMEABILITY-INCREASING, LIPID-BINDING, | | |
| KEYWDS | 2 | LIPOPOLYSACCHARIDE-BINDING, ANTIBIOTIC | | |
| EXPDTA | | X-RAY DIFFRACTION | | |
| AUTHOR | | G. KLEIGER, L. J. BEAMER, R. GROTHE, P. MALLICK, D. EISENBERG | | |
| REVDAT | 1 | 21-JUN-00 | 1EWF | 0 |

| JRNL | | AUTH G. KLEIGER, L. J. BEAMER, R. GROTHE, P. MALLICK, D. EISENBERG | |
|---|---|---|---|
| JRNL | | TITL THE 1.7 ANGSTROM CRYSTAL STRUCTURE OF BPI: A STUDY | |
| JRNL | | TITL 2 OF HOW TWO DISSIMILAR AMINO ACID SEQUENCES CAN | |
| JRNL | | TITL 3 ADOPT THE SAME FOLD. | |
| JRNL | | REF J. MOL. BIOL. V. 299 1019 2000 | |
| JRNL | | REFN ASTM JMOBAK UK ISSN 0022-2836 | |
| REMARK | 1 | | |
| REMARK | 1 | REFERENCE 1 | |
| REMARK | 1 | AUTH | L. J. BEAMER, S. F. CARROLL, D. EISENBERG |
| REMARK | 1 | TITL | CRYSTAL STRUCTURE OF HUMAN BPI AND TWO BOUND |
| REMARK | 1 | TITL 2 | PHOSPHOLIPIDS AT 2.4 ANGSTROM RESOLUTION |

| | | | | | | |
|---|---|---|---|---|---|---|
| REMARK | 1 | REF | SCIENCE | V. 276 | 1861 | 1997 |

| REMARK | 1 | REFN ASTM SCIEAS US ISSN 0036-8075 | |
|---|---|---|---|
| REMARK | 2 | | |
| REMARK | 2 | RESOLUTION. 1.70 ANGSTROMS. | |
| REMARK | 3 | | |
| REMARK | 3 | REFINEMENT. | |
| REMARK | 3 | PROGRAM | CNS |
| REMARK | 3 | AUTHORS | BRUNGER, ADAMS, CLORE, DELANO, GROS, GROSSE- |
| REMARK | 3 | | KUNSTLEVE, JIANG, KUSZEWSKI, NILGES, PANNU, |
| REMARK | 3 | | READ, RICE, SIMONSON, WARREN |
| REMARK | 3 | | |
| REMARK | 3 | REFINEMENT TARGET | ENGH & HUBER |
| REMARK | 3 | | |
| REMARK | 3 | DATA USED IN REFINEMENT. | |

| | | | |
|---|---|---|---|
| REMARK | 3 | RESOLUTION RANGE HIGH (ANGSTROMS) | 1.70 |
| REMARK | 3 | RESOLUTION RANGE LOW (ANGSTROMS) | 50.00 |
| REMARK | 3 | DATA CUTOFF (SIGMA(F)) | 0.000 |
| REMARK | 3 | OUTLIER CUTOFF HIGH (RMS(ABS(F))) | NULL |
| REMARK | 3 | COMPLETENESS (WORKING + TEST) (%) | 94.2 |
| REMARK | 3 | NUMBER OF REFLECTIONS | 47197 |
| REMARK | 3 | | |
| REMARK | 3 | | |
| REMARK | 3 | FIT TO DATA USED IN REFINEMENT. | |
| REMARK | 3 | CROSS-VALIDATION METHOD | NULL |
| REMARK | 3 | FREE R VALUE TEST SET SELECTION | RANDOM SELECTION OF |
| REMARK | 3 | | 10 PERCENT OF ALL DATA |
| REMARK | 3 | R VALUE (WORKING SET) | 0.198 |
| REMARK | 3 | FREE R VALUE | 0.250 |
| REMARK | 3 | FREE R VALUE TEST SET SIZE (%) | NULL |
| REMARK | 3 | FREE R VALUE TEST SET COUNT | 4755 |
| REMARK | 3 | ESTIMATED ERROR OF FREE R VALUE | NULL |
| REMARK | 3 | | |
| REMARK | 3 | FIT IN THE HIGHEST RESOLUTION BIN. | |
| REMARK | 3 | TOTAL NUMBER OF BINS USED | NULL |
| REMARK | 3 | BIN RESOLUTION RANGE HIGH (A) | NULL |
| REMARK | 3 | BIN RESOLUTION RANGE LOW (A) | NULL |
| REMARK | 3 | BIN COMPLETENESS (WORKING + TEST) | |
| | | (%) | NULL |
| REMARK | 3 | REFLECTIONS IN BIN (WORKING SET) | NULL |
| REMARK | 3 | BIN R VALUE (WORKING SET) | NULL |
| REMARK | 3 | BIN FREE R VALUE | NULL |
| REMARK | 3 | BIN FREE R VALUE TEST SET SIZE (%) | NULL |
| REMARK | 3 | BIN FREE R VALUE TEST SET COUNT | NULL |
| REMARK | 3 | ESTIMATED ERROR OF BIN FREE R VALUE | NULL |
| REMARK | 3 | | |
| REMARK | 3 | NUMBER OF NON-HYDROGEN ATOMS | |
| | | USED IN REFINEMENT. | |
| REMARK | 3 | PROTEIN ATOMS | 3663 |
| REMARK | 3 | NUCLEIC ACID ATOMS | 0 |
| REMARK | 3 | HETEROGEN ATOMS | 97 |
| REMARK | 3 | SOLVENT ATOMS | 387 |
| REMARK | 3 | | |
| REMARK | 3 | B VALUES. | |
| REMARK | 3 | FROM WILSON PLOT (A**2) | 24.00 |
| REMARK | 3 | MEAN B VALUE (OVERALL, A**2) | NULL |
| REMARK | 3 | OVERALL ANISOTROPIC B VALUE. | |
| REMARK | 3 | B11 (A**2) | NULL |
| REMARK | 3 | B22 (A**2) | NULL |
| REMARK | 3 | B33 (A**2) | NULL |
| REMARK | 3 | B12 (A**2) | NULL |
| REMARK | 3 | B13 (A**2) | NULL |
| REMARK | 3 | B23 (A**2) | NULL |
| REMARK | 3 | | |
| REMARK | 3 | ESTIMATED COORDINATE ERROR. | |
| REMARK | 3 | ESD FROM LUZZATI PLOT (A) | NULL |
| REMARK | 3 | ESD FROM SIGMAA (A) | NULL |
| REMARK | 3 | LOW RESOLUTION CUTOFF (A) | NULL |
| REMARK | 3 | | |
| REMARK | 3 | CROSS-VALIDATED ESTIMATED | |
| | | COORDINATE ERROR. | |
| REMARK | 3 | ESD FROM C-V LUZZATI PLOT (A) | NULL |
| REMARK | 3 | ESD FROM C-V SIGMAA (A) | NULL |
| REMARK | 3 | | |
| REMARK | 3 | RMS DEVIATIONS FROM IDEAL VALUES. | |
| REMARK | 3 | BOND LENGTHS (A) | 0.017 |
| REMARK | 3 | BOND ANGLES (DEGREES) | 2.00 |
| REMARK | 3 | DIHEDRAL ANGLES (DEGREES) | NULL |
| REMARK | 3 | IMPROPER ANGLES (DEGREES) | NULL |
| REMARK | 3 | | |
| REMARK | 3 | ISOTROPIC THERMAL MODEL | NULL |
| REMARK | 3 | | |

| | | | | |
|---|---|---|---|---|
| REMARK | 3 | ISOTROPIC THERMAL FACTOR RESTRAINTS. | RMS | SIGMA |
| REMARK | 3 | MAIN-CHAIN BOND (A**2) | NULL | NULL |
| REMARK | 3 | MAIN-CHAIN ANGLE (A**2) | NULL | NULL |
| REMARK | 3 | SIDE-CHAIN BOND (A**2) | NULL | NULL |
| REMARK | 3 | SIDE-CHAIN ANGLE (A**2) | NULL | NULL |
| REMARK | 3 | | | |
| REMARK | 3 | | | |

| REMARK | 3 | BULK SOLVENT MODELING. | |
|---|---|---|---|
| REMARK | 3 | METHOD USED | NULL |
| REMARK | 3 | KSOL | NULL |
| REMARK | 3 | BSOL | NULL |
| REMARK | 3 | | |
| REMARK | 3 | NCS MODEL | NULL |
| REMARK | 3 | | |

| | | | | |
|---|---|---|---|---|
| REMARK | 3 | NCS RESTRAINTS. | RMS | SIGMA/WEIGHT |
| REMARK | 3 | GROUP 1 POSITIONAL (A) | NULL | NULL |
| REMARK | 3 | GROUP 1 B-FACTOR (A**2) | NULL | NULL |

| | | | |
|---|---|---|---|
| REMARK | 3 | | |
| REMARK | 3 | PARAMETER FILE 1 | NULL |
| REMARK | 3 | TOPOLOGY FILE 1 | NULL |
| REMARK | 3 | | |
| REMARK | 3 | OTHER REFINEMENT REMARKS | NULL |
| REMARK | 4 | | |

| REMARK | 4 | 1EWF COMPLIES WITH FORMAT V. 2.3, 09-JULY-1998 | |
|---|---|---|---|
| REMARK | 6 | | |
| REMARK | 6 | ELECTRON DENSITY WAS MISSING OR DIFFUSE FOR THE | |
| REMARK | 6 | ATOMS LISTED IN REMARK 470. | |
| REMARK | 100 | | |
| REMARK | 100 | THIS ENTRY HAS BEEN PROCESSED BY RCSB ON 05-MAY-2000. | |
| REMARK | 100 | THE RCSB ID CODE IS RCSB010960. | |
| REMARK | 200 | | |
| REMARK | 200 | EXPERIMENTAL DETAILS | |
| REMARK | 200 | EXPERIMENT TYPE | X-RAY DIFFRACTION |
| REMARK | 200 | DATE OF DATA COLLECTION | 06-FEB-1998 |
| REMARK | 200 | TEMPERATURE (KELVIN) | 93.0 |
| REMARK | 200 | PH | 6.80 |
| REMARK | 200 | NUMBER OF CRYSTALS USED | 1 |
| REMARK | 200 | | |
| REMARK | 200 | SYNCHROTRON (Y/N) | Y |
| REMARK | 200 | RADIATION SOURCE | NSLS |
| REMARK | 200 | BEAMLINE | X12B |
| REMARK | 200 | X-RAY GENERATOR MODEL | NULL |
| REMARK | 200 | MONOCHROMATIC OR LAUE (M/L) | M |
| REMARK | 200 | WAVELENGTH OR RANGE (A) | 0.975 |
| REMARK | 200 | MONOCHROMATOR | NULL |
| REMARK | 200 | OPTICS | NULL |
| REMARK | 200 | | |
| REMARK | 200 | DETECTOR TYPE | CCD |
| REMARK | 200 | DETECTOR MANUFACTURER | QUANTUM IV |
| REMARK | 200 | INTENSITY - INTEGRATION SOFTWARE | DENZO |
| REMARK | 200 | DATA SCALING SOFTWARE | SCALEPACK |
| REMARK | 200 | | |
| REMARK | 200 | NUMBER OF UNIQUE REFLECTIONS | 47198 |
| REMARK | 200 | RESOLUTION RANGE HIGH (A) | 1.700 |
| REMARK | 200 | RESOLUTION RANGE LOW (A) | 100.000 |
| REMARK | 200 | REJECTION CRITERIA (SIGMA(I)) | −3.000 |
| REMARK | 200 | | |
| REMARK | 200 | OVERALL. | |
| REMARK | 200 | COMPLETENESS FOR RANGE (%) | 94.2 |
| REMARK | 200 | DATA REDUNDANCY | 2.300 |
| REMARK | 200 | R MERGE (I) | 0.04800 |
| REMARK | 200 | R SYM (I) | NULL |
| REMARK | 200 | <I/SIGMA(I)> FOR THE DATA SET | 16.7000 |
| REMARK | 200 | | |
| REMARK | 200 | IN THE HIGHEST RESOLUTION SHELL. | |
| REMARK | 200 | HIGHEST RESOLUTION SHELL, RANGE HIGH (A) | 1.70 |
| REMARK | 200 | HIGHEST RESOLUTION SHELL, RANGE LOW (A) | 1.76 |
| REMARK | 200 | COMPLETENESS FOR SHELL (%) | 67.0 |
| REMARK | 200 | DATA REDUNDANCY IN SHELL | 1.30 |
| REMARK | 200 | R MERGE FOR SHELL (I) | 0.20100 |
| REMARK | 200 | R SYM FOR SHELL (I) | NULL |
| REMARK | 200 | <I/SIGMA(I)> FOR SHELL | NULL |
| REMARK | 200 | | |
| REMARK | 200 | DIFFRACTION PROTOCOL | SINGLE WAVELENGTH |
| REMARK | 200 | METHOD USED TO DETERMINE THE STRUCTURE | NULL |
| REMARK | 200 | SOFTWARE USED | X-PLOR |
| REMARK | 200 | STARTING MODEL | NULL |
| REMARK | 200 | | |
| REMARK | 200 | REMARK | NULL |
| REMARK | 280 | | |
| REMARK | 280 | CRYSTAL | |
| REMARK | 280 | SOLVENT CONTENT, VS (%) | NULL |
| REMARK | 280 | MATTHEWS COEFFICIENT, VM (ANGSTROMS**3/DA) | NULL |
| REMARK | 280 | | |

| | | | |
|---|---|---|---|
| REMARK | 280 | CRYSTALLIZATION CONDITIONS | 12% PEG 6000, 0.2 M MG |

| REMARK | 280 | ACETATE, 0.1 M NA CACODYLATE, PH 6.8 | |
|---|---|---|---|
| REMARK | 290 | | |
| REMARK | 290 | CRYSTALLOGRAPHIC SYMMETRY | |
| REMARK | 290 | SYMMETRY OPERATORS FOR SPACE GROUP C 1 2 1 | |
| REMARK | 290 | | |
| REMARK | 290 | SYMOP | SYMMETRY |
| REMARK | 290 | NNNMMM | OPERATOR |
| REMARK | 290 | 1555 | X,Y,Z |
| REMARK | 290 | 2555 | −X,Y,−Z |
| REMARK | 290 | 3555 | 1/2 + X, 1/2 + Y, Z |
| REMARK | 290 | 4555 | 1/2 − X, 1/2 + Y, −Z |
| REMARK | 290 | | |

| REMARK | 290 | WHERE NNN -> OPERATOR NUMBER | | | | | |
|---|---|---|---|---|---|---|---|
| REMARK | 290 | MMM -> TRANSLATION VECTOR | | | | | |
| REMARK | 290 | | | | | | |
| REMARK | 290 | CRYSTALLOGRAPHIC SYMMETRY TRANSFORMATIONS | | | | | |
| REMARK | 290 | THE FOLLOWING TRANSFORMATIONS OPERATE ON THE ATOM/HETATM | | | | | |
| REMARK | 290 | RECORDS IN THIS ENTRY TO PRODUCE CRYSTALLOGRAPHICALLY | | | | | |
| REMARK | 290 | RELATED MOLECULES. | | | | | |
| REMARK | 290 | SMTRY1 | 1 | 1.000000 | 0.000000 | 0.000000 | 0.00000 |
| REMARK | 290 | SMTRY2 | 1 | 0.000000 | 1.000000 | 0.000000 | 0.00000 |
| REMARK | 290 | SMTRY3 | 1 | 0.000000 | 0.000000 | 1.000000 | 0.00000 |
| REMARK | 290 | SMTRY1 | 2 | −1.000000 | 0.000000 | 0.000000 | 0.00000 |
| REMARK | 290 | SMTRY2 | 2 | 0.000000 | 1.000000 | 0.000000 | 0.00000 |
| REMARK | 290 | SMTRY3 | 2 | 0.000000 | 0.000000 | −1.000000 | 0.00000 |
| REMARK | 290 | SMTRY1 | 3 | 1.000000 | 0.000000 | 0.000000 | 92.16000 |
| REMARK | 290 | SMTRY2 | 3 | 0.000000 | 1.000000 | 0.000000 | 15.61500 |
| REMARK | 290 | SMTRY3 | 3 | 0.000000 | 0.000000 | 1.000000 | 0.00000 |
| REMARK | 290 | SMTRY1 | 4 | −1.000000 | 0.000000 | 0.000000 | 92.16000 |
| REMARK | 290 | SMTRY2 | 4 | 0.000000 | 1.000000 | 0.000000 | 15.61500 |
| REMARK | 290 | SMTRY3 | 4 | 0.000000 | 0.000000 | −1.000000 | 0.00000 |
| REMARK | 290 | | | | | | |

| | | | |
|---|---|---|---|
| REMARK | 290 | REMARK | NULL |
| REMARK | 300 | | |
| REMARK | 300 | BIOMOLECULE | 1 |

| REMARK | 300 | THIS ENTRY CONTAINS THE CRYSTALLOGRAPHIC ASYMMETRIC UNIT | | | | | |
|---|---|---|---|---|---|---|---|
| REMARK | 300 | WHICH CONSISTS OF 1 CHAIN(S). SEE REMARK 350 FOR | | | | | |
| REMARK | 300 | INFORMATION ON GENERATING THE BIOLOGICAL MOLECULE(S). | | | | | |
| REMARK | 350 | | | | | | |
| REMARK | 350 | GENERATING THE BIOMOLECULE | | | | | |
| REMARK | 350 | COORDINATES FOR A COMPLETE MULTIMER REPRESENTING THE KNOWN | | | | | |
| REMARK | 350 | BIOLOGICALLY SIGNIFICANT OLIGOMERIZATION STATE OF THE | | | | | |
| REMARK | 350 | MOLECULE CAN BE GENERATED BY APPLYING BIOMT TRANSFORMATIONS | | | | | |
| REMARK | 350 | GIVEN BELOW. BOTH NON-CRYSTALLOGRAPHIC AND | | | | | |
| REMARK | 350 | CRYSTALLOGRAPHIC OPERATIONS ARE GIVEN. | | | | | |
| REMARK | 350 | | | | | | |
| REMARK | 350 | BIOMOLECULE 1 | | | | | |
| REMARK | 350 | APPLY THE FOLLOWING TO CHAINS: A | | | | | |
| REMARK | 350 | BIOMT1 | 1 | 1.000000 | 0.000000 | 0.000000 | 0.00000 |
| REMARK | 350 | BIOMT2 | 1 | 0.000000 | 1.000000 | 0.000000 | 0.00000 |
| REMARK | 350 | BIOMT3 | 1 | 0.000000 | 0.000000 | 1.000000 | 0.00000 |
| REMARK | 470 | | | | | | |

| REMARK | 470 | MISSING ATOM | | | | | | | |
|---|---|---|---|---|---|---|---|---|---|
| REMARK | 470 | THE FOLLOWING RESIDUES HAVE MISSING ATOMS (M = MODEL NUMBER; | | | | | | | |
| REMARK | 470 | RES = RESIDUE NAME; C = CHAIN IDENTIFIER; SSEQ = SEQUENCE NUMBER; | | | | | | | |
| REMARK | 470 | I = INSERTION CODE): | | | | | | | |
| REMARK | 470 | M RES CSSEQI ATOMS | | | | | | | |
| REMARK | 470 | VAL | A | 1 | CG1 | CG2 | | | |
| REMARK | 470 | LYS | A | 33 | CG | CD | CE | NZ | |
| REMARK | 470 | LYS | A | 44 | CG | CD | CE | NZ | |
| REMARK | 470 | HIS | A | 45 | CG | ND1 | CD2 | CE1 | NE2 |
| REMARK | 470 | LYS | A | 86 | CD | CE | NZ | | |
| REMARK | 470 | LYS | A | 95 | CG | CD | CE | NZ | |
| REMARK | 470 | LYS | A | 99 | CG | CD | CE | NZ | |
| REMARK | 470 | LYS | A | 118 | CG | CD | CE | NZ | |
| REMARK | 470 | ASN | A | 232 | CG | OD1 | ND2 | | |
| REMARK | 470 | HIS | A | 233 | CG | ND1 | CD2 | CE1 | NE2 |
| REMARK | 470 | HIS | A | 234 | CG | ND1 | CD2 | CE1 | NE2 |
| REMARK | 470 | ASN | A | 235 | CG | OD1 | ND2 | | |
| REMARK | 500 | | | | | | | | |

| REMARK | 500 | GEOMETRY AND STEREOCHEMISTRY | | | | | | | | |
|---|---|---|---|---|---|---|---|---|---|---|
| REMARK | 500 | SUBTOPIC: CLOSE CONTACTS IN SAME ASYMMETRIC UNIT | | | | | | | | |
| REMARK | 500 | | | | | | | | | |
| REMARK | 500 | THE FOLLOWING ATOMS ARE IN CLOSE CONTACT. | | | | | | | | |
| REMARK | 500 | | | | | | | | | |
| REMARK | 500 | ATM1 | RES | C | SSEQI | ATM2 | RES | C | SSEQI | |
| REMARK | 500 | O | HOH | | 626 | O | HOH | | 774 | 2.07 |
| REMARK | 500 | N | ASN | A | 82 | O | HOH | | 665 | 2.09 |
| REMARK | 500 | O | HOH | | 609 | O | HOH | | 823 | 2.12 |
| REMARK | 500 | O | HOH | | 855 | O | HOH | | 779 | 2.14 |
| REMARK | 500 | O | HOH | | 870 | O | PRO | A | 303 | 2.15 |
| REMARK | 500 | O | LEU | A | 302 | O | HOH | | 930 | 2.17 |
| REMARK | 500 | O | HOH | | 906 | O | HOH | | 700 | 2.19 |

| REMARK | 500 | | | | | | | | | | |
|---|---|---|---|---|---|---|---|---|---|---|---|
| REMARK | 500 | GEOMETRY AND STEREOCHEMISTRY | | | | | | | | | |
| REMARK | 500 | SUBTOPIC: COVALENT BOND LENGTHS | | | | | | | | | |
| REMARK | 500 | | | | | | | | | | |
| REMARK | 500 | THE STEREOCHEMICAL PARAMETERS OF THE FOLLOWING RESIDUES | | | | | | | | | |
| REMARK | 500 | HAVE VALUES WHICH DEVIATE FROM EXPECTED VALUES BY MORE | | | | | | | | | |
| REMARK | 500 | THAN 6 * RMSD (M = MODEL NUMBER; RES = RESIDUE NAME; C = CHAIN | | | | | | | | | |
| REMARK | 500 | IDENTIFIER; SSEQ = SEQUENCE NUMBER; I = INSERTION CODE). | | | | | | | | | |
| REMARK | 500 | | | | | | | | | | |
| REMARK | 500 | STANDARD TABLE: | | | | | | | | | |
| REMARK | 500 | FORMAT: (10X, I3, 1X, 2 (A3, 1X, A1, I4, A1, 1X, A4, 3X), F6.3) | | | | | | | | | |
| REMARK | 500 | | | | | | | | | | |
| REMARK | 500 | EXPECTED VALUES: ENGH AND HUBER, 1991 | | | | | | | | | |
| REMARK | 500 | | | | | | | | | | |
| REMARK | 500 | M RES C SSEQI ATM1 RES C SSEQI ATM2 DEVIATION | | | | | | | | | |
| REMARK | 500 | | MET | A | 70 | CE | MET | A | 70 | SD | −0.230 |
| REMARK | 500 | | MET | A | 170 | CE | MET | A | 170 | SD | −0.130 |
| REMARK | 500 | | VAL | A | 269 | CG1 | VAL | A | 269 | CB | 0.103 |
| REMARK | 500 | | MET | A | 278 | CE | MET | A | 278 | SD | −0.119 |
| REMARK | 500 | | MET | A | 405 | CE | MET | A | 405 | SD | −0.192 |

| REMARK | 500 | | | | | | | |
|---|---|---|---|---|---|---|---|---|
| REMARK | 500 | GEOMETRY AND STEREOCHEMISTRY | | | | | | |
| REMARK | 500 | SUBTOPIC: COVALENT BOND ANGLES | | | | | | |
| REMARK | 500 | | | | | | | |
| REMARK | 500 | THE STEREOCHEMICAL PARAMETERS OF THE FOLLOWING RESIDUES | | | | | | |
| REMARK | 500 | HAVE VALUES WHICH DEVIATE FROM EXPECTED VALUES BY MORE | | | | | | |
| REMARK | 500 | THAN 6 * RMSD (M = MODEL NUMBER; RES = RESIDUE NAME; C = CHAIN | | | | | | |
| REMARK | 500 | IDENTIFIER; SSEQ = SEQUENCE NUMBER; I = INSERTION CODE). | | | | | | |
| REMARK | 500 | | | | | | | |
| REMARK | 500 | STANDARD TABLE: | | | | | | |
| REMARK | 500 | FORMAT: (10X, I3, 1X, A3, 1X, A1, I4, A1, 3 (1X, A4, 2X), (12X, F5.1) | | | | | | |
| REMARK | 500 | | | | | | | |
| REMARK | 500 | EXPECTED VALUES: ENGH AND HUBER, 1991 | | | | | | |
| REMARK | 500 | | | | | | | |
| REMARK | 500 | M | RES | C | SSEQI | ATM1 | ATM2 | ATM3 |

| | | | | | | | | |
|---|---|---|---|---|---|---|---|---|
| REMARK | 500 | LYS | A | 42 | N | CA | C | ANGL. DEV. = −15.2 DEGREES |
| REMARK | 500 | LYS | A | 44 | N | CA | C | ANGL. DEV. = 18.4 DEGREES |
| REMARK | 500 | HIS | A | 45 | N | CA | C | ANGL. DEV. = −27.6 DEGREES |
| REMARK | 500 | HIS | A | 45 | C | N | CA | ANGL. DEV. = 12.1 DEGREES |
| REMARK | 500 | GLY | A | 47 | N | CA | C | ANGL. DEV. = 17.7 DEGREES |
| REMARK | 500 | LYS | A | 48 | N | CA | C | ANGL. DEV. = 20.9 DEGREES |
| REMARK | 500 | GLY | A | 49 | N | CA | C | ANGL. DEV. = 28.6 DEGREES |
| REMARK | 500 | SER | A | 81 | N | CA | C | ANGL. DEV. = 14.5 DEGREES |
| REMARK | 500 | | | | | | | |

| REMARK | 500 | GEOMETRY AND STEREOCHEMISTRY | | | | | |
|---|---|---|---|---|---|---|---|
| REMARK | 500 | SUBTOPIC: TORSION ANGLES | | | | | |
| REMARK | 500 | | | | | | |
| REMARK | 500 | TORSION ANGLES OUTSIDE THE EXPECTED RAMACHANDRAN REGIONS: | | | | | |
| REMARK | 500 | (M = MODEL NUMBER; RES = RESIDUE NAME; C = CHAIN IDENTIFIER; | | | | | |
| REMARK | 500 | SSEQ = SEQUENCE NUMBER; I = INSERTION CODE). | | | | | |
| REMARK | 500 | | | | | | |
| REMARK | 500 | STANDARD TABLE: | | | | | |
| REMARK | 500 | FORMAT: (10X, I3, 1X, A3, 1X, A1, I4, A1, 4X, F7.2, 3X, F7.2) | | | | | |
| REMARK | 500 | | | | | | |
| REMARK | 500 | M | RES | C | SSEQI | PSI | PHI |

| | | | | | | |
|---|---|---|---|---|---|---|
| REMARK | 500 | HIS | A | 45 | 146.65 | −9.33 |
| REMARK | 500 | LEU | A | 46 | 141.72 | 106.25 |
| REMARK | 500 | ARG | A | 96 | 59.46 | −115.40 |

| | | |
|---|---|---|
| REMARK | 600 | |
| REMARK | 600 | HETEROGEN |
| REMARK | 600 | THE HEAD GROUP OF PC2 577 IS MISSING IN THE ELECTRON |
| REMARK | 600 | DENSITY MAPS. |
| REMARK | 900 | |
| REMARK | 900 | RELATED ENTRIES |
| REMARK | 900 | RELATED ID: 1BP1 RELATED DB: PDB |
| REMARK | 900 | CRYSTAL STRUCTURE OF HUMAN BPI AT ROOM TEMPERATURE |
| REMARK | 999 | |
| REMARK | 999 | SEQUENCE |
| REMARK | 999 | A NATURALLY OCCURING POLYMORPHISM EXISTS AT RESIDUE |
| REMARK | 999 | 185 FOR HUMAN BPI. THE CLONE USED FOR EXPRESSION |
| REMARK | 999 | OF BPI HAS GLU AT THIS POSITION, ALTHOUGH THE CLONE |
| REMARK | 999 | FOR THE SWISSPROT ENTRY HAS LYS AT THE SAME POSITION. |

| | | | | | | | | | |
|---|---|---|---|---|---|---|---|---|---|
| DBREF | 1EWF | A | 1 | 456 | SWS | P17213 | BPI_HUMAN | 28 | 483 |

| | | | | | | | | | |
|---|---|---|---|---|---|---|---|---|---|
| SEQADV | 1EWF | GLU | A | 185 | SWS | P17213 | LYS | 212 | SEE REMARK 999 |
| SEQADV | 1EWF | ALA | A | 351 | SWS | P17213 | SER | 378 | ENGINEERED |

| | | | | | | | | | | | | | | | | |
|---|---|---|---|---|---|---|---|---|---|---|---|---|---|---|---|---|
| SEQRES | 1 | A | 456 | VAL | ASN | PRO | GLY | VAL | VAL | VAL | ARG | ILE | SER | GLN | LYS | GLY |
| SEQRES | 2 | A | 456 | LEU | ASP | TYR | ALA | SER | GLN | GLN | GLY | THR | ALA | ALA | LEU | GLN |
| SEQRES | 3 | A | 456 | LYS | GLU | LEU | LYS | ARG | ILE | LYS | ILE | PRO | ASP | TYR | SER | ASP |
| SEQRES | 4 | A | 456 | SER | PHE | LYS | ILE | LYS | HIS | LEU | GLY | LYS | GLY | HIS | TYR | SER |
| SEQRES | 5 | A | 456 | PHE | TYR | SER | MET | ASP | ILE | ARG | GLU | PHE | GLN | LEU | PRO | SER |
| SEQRES | 6 | A | 456 | SER | GLN | ILE | SER | MET | VAL | PRO | ASN | VAL | GLY | LEU | LYS | PHE |
| SEQRES | 7 | A | 456 | SER | ILE | SER | ASN | ALA | ASN | ILE | LYS | ILE | SER | GLY | LYS | TRP |
| SEQRES | 8 | A | 456 | LYS | ALA | GLN | LYS | ARG | PHE | LEU | LYS | MET | SER | GLY | ASN | PHE |
| SEQRES | 9 | A | 456 | ASP | LEU | SER | ILE | GLU | GLY | MET | SER | ILE | SER | ALA | ASP | LEU |
| SEQRES | 10 | A | 456 | LYS | LEU | GLY | SER | ASN | PRO | THR | SER | GLY | LYS | PRO | THR | ILE |
| SEORES | 11 | A | 456 | THR | CYS | SER | SER | CYS | SER | SER | HIS | ILE | ASN | SER | VAL | HIS |
| SEQRES | 12 | A | 456 | VAL | HIS | ILE | SER | LYS | SER | LYS | VAL | GLY | TRP | LEU | ILE | GLN |
| SEQRES | 13 | A | 456 | LEU | PHE | HIS | LYS | LYS | ILE | GLU | SER | ALA | LEU | ARG | ASN | LYS |
| SEQRES | 14 | A | 456 | MET | ASN | SER | GLN | VAL | CYS | GLU | LYS | VAL | THR | ASN | SER | VAL |
| SEQRES | 15 | A | 456 | SER | SER | GLU | LEU | GLN | PRO | TYR | PHE | GLN | THR | LEU | PRO | VAL |
| SEQRES | 16 | A | 456 | MET | THR | LYS | ILE | ASP | SER | VAL | ALA | GLY | ILE | ASN | TYR | GLY |
| SEQRES | 17 | A | 456 | LEU | VAL | ALA | PRO | PRO | ALA | THR | THR | ALA | GLU | THR | LEU | ASP |
| SEQRES | 18 | A | 456 | VAL | GLN | MET | LYS | GLY | GLU | PHE | TYR | SER | GLU | ASN | HIS | HIS |
| SEQRES | 19 | A | 456 | ASN | PRO | PRO | PRO | PHE | ALA | PRO | PRO | VAL | MET | GLU | PHE | PRO |
| SEQRES | 20 | A | 456 | ALA | ALA | HIS | ASP | ARG | MET | VAL | TYR | LEU | GLY | LEU | SER | ASP |
| SEQRES | 21 | A | 456 | TYR | PHE | PHE | ASN | THR | ALA | GLY | LEU | VAL | TYR | GLN | GLU | ALA |
| SEQRES | 22 | A | 456 | GLY | VAL | LEU | LYS | MET | THR | LEU | ARG | ASP | ASP | MET | ILE | PRO |
| SEQRES | 23 | A | 456 | LYS | GLU | SER | LYS | PHE | ARG | LEU | THR | THR | LYS | PHE | PHE | GLY |
| SEQRES | 24 | A | 456 | THR | PHE | LEU | PRO | GLU | VAL | ALA | LYS | LYS | PHE | PRO | ASN | MET |
| SEQRES | 25 | A | 456 | LYS | ILE | GLN | ILE | HIS | VAL | SER | ALA | SER | THR | PRO | PRO | HIS |
| SEQRES | 26 | A | 456 | LEU | SER | VAL | GLN | PRO | THR | GLY | LEU | THR | PHE | TYR | PRO | ALA |
| SEORES | 27 | A | 456 | VAL | ASP | VAL | GLN | ALA | PHE | ALA | VAL | LEU | PRO | ASN | SER | ALA |
| SEQRES | 28 | A | 456 | LEU | ALA | SER | LEU | PHE | LEU | ILE | GLY | MET | HIS | THR | THR | GLY |
| SEQRES | 29 | A | 456 | SER | MET | GLU | VAL | SER | ALA | GLU | SER | ASN | ARG | LEU | VAL | GLY |
| SEQRES | 30 | A | 456 | GLU | LEU | LYS | LEU | ASP | ARG | LEU | LEU | LEU | GLU | LEU | LYS | HIS |
| SEQRES | 31 | A | 456 | SER | ASN | ILE | GLY | PRO | PHE | PRO | VAL | GLU | LEU | LEU | GLN | ASP |
| SEQRES | 32 | A | 456 | ILE | MET | ASN | TYR | ILE | VAL | PRO | ILE | LEU | VAL | LEU | PRO | ARG |
| SEQRES | 33 | A | 456 | VAL | ASN | GLU | LYS | LEU | GLN | LYS | GLY | PHE | PRO | LEU | PRO | THR |
| SEQRES | 34 | A | 456 | PRO | ALA | ARG | VAL | GLN | LEU | TYR | ASN | VAL | VAL | LEU | GLN | PRO |
| SEQRES | 35 | A | 456 | HIS | GLN | ASN | PHE | LEU | LEU | PHE | GLY | ALA | ASP | VAL | VAL | TYR |
| SEQRES | 36 | A | 456 | LYS | | | | | | | | | | | | |

| | | | |
|---|---|---|---|
| HET | PC2 | 577 | 54 |
| HET | PC2 | 578 | 43 |

| | | |
|---|---|---|
| HETNAM | PC2 | DI-STEAROYL-3-SN-PHOSPHATIDYLCHOLINE |

| | | | |
|---|---|---|---|
| FORMUL | 2 | PC2 | 2(C44 H89 N1 O8 P1 1 + ) |
| FORMUL | 4 | HOH | *387(H2 O1) |

| | | | | | | | | | | |
|---|---|---|---|---|---|---|---|---|---|---|
| HELIX | 1 | 1 | GLN | A | 11 | LYS | A | 30 | 1 | 20 |
| HELIX | 2 | 2 | LYS | A | 148 | LYS | A | 150 | 5 | 3 |
| HELIX | 3 | 3 | VAL | A | 151 | ILE | A | 162 | 1 | 12 |
| HELIX | 4 | 4 | ILE | A | 162 | GLU | A | 185 | 1 | 24 |
| HELIX | 5 | 5 | GLU | A | 185 | GLN | A | 191 | 1 | 7 |
| HELIX | 6 | 6 | ASP | A | 260 | ALA | A | 273 | 1 | 14 |
| HELIX | 7 | 7 | ASP | A | 283 | ILE | A | 285 | 5 | 3 |
| HELIX | 8 | 8 | THR | A | 294 | THR | A | 300 | 1 | 7 |
| HELIX | 9 | 9 | GLU | A | 304 | PHE | A | 309 | 1 | 6 |
| HELIX | 10 | 10 | PRO | A | 397 | LEU | A | 400 | 5 | 4 |
| HELIX | 11 | 11 | LEU | A | 401 | VAL | A | 413 | 1 | 13 |
| HELIX | 12 | 12 | VAL | A | 413 | GLY | A | 424 | 1 | 12 |
| SHEET | 1 | A | 6 | ALA | A | 214 | THR | A | 215 | 0 |

| | | | | | | | | | | | | | | | | | | |
|---|---|---|---|---|---|---|---|---|---|---|---|---|---|---|---|---|---|---|
| SHEET | 2 | A | 6 | THR | A | 219 | MET | A | 224 | −1 | N | ASP | A | 221 | O | ALA | A | 214 |
| SHEET | 3 | A | 6 | VAL | A | 5 | SER | A | 10 | −1 | O | VAL | A | 5 | N | MET | A | 224 |
| SHEET | 4 | A | 6 | VAL | A | 254 | SER | A | 259 | −1 | O | TYR | A | 255 | N | ARG | A | 8 |
| SHEET | 5 | A | 6 | PHE | A | 446 | TYR | A | 455 | −1 | O | LEU | A | 447 | N | LEU | A | 258 |
| SHEET | 6 | A | 6 | VAL | A | 433 | HIS | A | 443 | −1 | N | GLN | A | 434 | O | VAL | A | 454 |
| SHEET | 1 | B | 9 | TYR | A | 37 | SER | A | 40 | 0 | | | | | | | | |
| SHEET | 2 | B | 9 | HIS | A | 50 | GLN | A | 62 | −1 | N | TYR | A | 51 | O | ASP | A | 39 |
| SHEET | 3 | B | 9 | GLY | A | 75 | LYS | A | 95 | −1 | O | ASN | A | 84 | N | GLN | A | 62 |
| SHEET | 4 | B | 9 | SER | A | 66 | VAL | A | 71 | −1 | O | GLN | A | 67 | N | SER | A | 79 |
| SHEET | 5 | B | 9 | GLY | A | 75 | LYS | A | 95 | −1 | O | GLY | A | 75 | N | VAL | A | 71 |
| SHEET | 6 | B | 9 | LEU | A | 98 | ASN | A | 122 | −1 | N | LEU | A | 98 | O | LYS | A | 95 |
| SHEET | 7 | B | 9 | LYS | A | 127 | HIS | A | 138 | −1 | O | LYS | A | 127 | N | ASN | A | 122 |
| SHEET | 8 | B | 9 | LEU | A | 98 | ASN | A | 122 | −1 | O | SER | A | 112 | N | HIS | A | 138 |
| SHEET | 9 | B | 9 | SER | A | 141 | HIS | A | 145 | −1 | O | SER | A | 141 | N | GLU | A | 109 |
| SHEET | 1 | C | 3 | MET | A | 196 | LYS | A | 198 | 0 | | | | | | | | |
| SHEET | 2 | C | 3 | GLY | A | 204 | ASN | A | 206 | −1 | O | ILE | A | 205 | N | THR | A | 197 |
| SHEET | 3 | C | 3 | GLU | A | 227 | TYR | A | 229 | −1 | O | GLU | A | 227 | N | ASN | A | 206 |
| SHEET | 1 | D | 5 | LYS | A | 277 | ARG | A | 281 | 0 | | | | | | | | |
| SHEET | 2 | D | 5 | ILE | A | 314 | SER | A | 319 | −1 | O | ILE | A | 316 | N | LEU | A | 280 |
| SHEET | 3 | D | 5 | ALA | A | 338 | VAL | A | 346 | −1 | N | ASP | A | 340 | O | SER | A | 319 |
| SHEET | 4 | D | 5 | LEU | A | 352 | HIS | A | 361 | −1 | N | ALA | A | 353 | O | ALA | A | 345 |
| SHEET | 5 | D | 5 | LEU | A | 385 | SER | A | 391 | −1 | N | LEU | A | 385 | O | HIS | A | 361 |

| | | | | | | | |
|---|---|---|---|---|---|---|---|
| SSBOND | 1 | CYS | A | 135 | CYS | A | 175 |

| | | | | | | | | |
|---|---|---|---|---|---|---|---|---|
| CRYST1 | 184.320 | 31.230 | 80.660 | 90.00 | 103.20 | 90.00 | C 1 2 1 | 4 |

| | | | | |
|---|---|---|---|---|
| ORIGX1 | 1.000000 | 0.000000 | 0.000000 | 0.00000 |
| ORIGX2 | 0.000000 | 1.000000 | 0.000000 | 0.00000 |
| ORIGX3 | 0.000000 | 0.000000 | 1.000000 | 0.00000 |
| SCALE1 | 0.005425 | 0.000000 | 0.001273 | 0.00000 |
| SCALE2 | 0.000000 | 0.032020 | 0.000000 | 0.00000 |
| SCALE3 | 0.000000 | 0.000000 | 0.012734 | 0.00000 |

### EXAMPLE 5

### b 3D-1D Environment Classes and Comparison of Structually Equivalent Positions in BPI Domains

Additional analyses were performed to compare N-terminal and C-terminal domains of BPI using 3D-1D environments to analyze environmentally conserved positions in the BPI alignment as a function of environment class, to analyze the structural roles of 3D-1D environmentally conserved positions with dissimilar residue with BPI domain alignment and to analyze the clustering of 3D-1D environmentally conserved positions in both the BPI fold and the lipid binding pockets. Initially, for these analyses, a structural and sequence alignment was done for the two BPI domains, which have substantially the same fold but different sequences and statistical analyses were performed.

Coordinates for residues 1 to 229 corresponding to the N-terminal domain of BPI and residues 251 to 456 corresponding to the C-terminal domain were used for rigid-body alignment of the two domains. The program Align_v2 was used for the superposition, which outputs a structure-based sequence alignment of the two domains [G. Cohen, et al., J. Mol. Biol., 190:593-604 (1986)]. All main-chain atoms were used for both the superposition and calculation of rmsd. Initially 173 positions were aligned by the program. Nine pairs had no structurally equivalent main-chain atoms within 5 Å and were not considered aligned. Therefore, the length of the entire BPI domain alignment is 164 positions.

Based on the results of the structural alignment of the N-terminal and C-terminal BPI domains, a sequence alignment was generated between the two domains [S. Benner et al., Prot. Eng. 7:1323 (1994)]. Conservation between the two domains was first examined at the residue level. A total of 21 pairs of residues are identical out of 164 aligned positions between the two domains, corresponding to a sequence identity of 13%. This level of sequence identity is significantly higher than would be expected for two independently generated random sequences of this length. The sequence identity is too low to allow sequence alignment methods to predict an alignment, and in fact the structural identity of the two domains was unsuspected prior to the 2.4 Å resolution structure.

To understand the fold identity of the two BPI domains in the absence of strong sequence similarity, positional similarities were examined using 3D-1D environment classes. 3D-1D profiles were generated for both the N-terminal domain and the C-terminal domain [J. Bowie, et al., Science, 253:164-170 (1991)]. The boundaries used for calculating environment classes are shown in Table 7. The secondary structure at each residue was characterized using the program DSSP [W. Kabsch and C. Sander, Biopolymers, 22:2577-2637 (1983)]. 3D-1D profiles and secondary structures were calculated in a similar manner for examples from the distant aligned protein structures (DAPS) database. The environments for each position in the two domains are shown on the structure-derived sequence alignment of the two domains.

TABLE 7

| | | | Area Buried | |
|---|---|---|---|---|
| Environment Class | | | (Å²) | Fraction Polar |
| H:B1 | E:B1 | C:B1 | Ab ≧ 114 | Fp ≦ 0.33 |
| H:B2 | E:B2 | C:B2 | Ab ≧ 114 | 0.33 ≦ Fp ≦ 0.46 |
| H:B3 | E:B3 | C:B3 | Ab ≧ 114 | 0.46 ≦ Fp ≦ 0.57 |
| H:P1 | E:P1 | C:P1 | 40 ≦ Ab ≦ 114 | 0.46 ≦ Fp ≦ 0.57 |
| H:P2 | E:P2 | C:P2 | 40 ≦ Ab ≦ 114 | 0.57 ≦ Fp ≦ 1.00 |
| H:E | E:E | C:E | Ab ≦ 40 | 0.00 ≦ Fp ≦ 1.00 |

Boundaries for area buried (Ab) and fraction of the area buried by polar atoms (Fp) used for defining 3D-1D environment classes. Each position defined by values of Ab and Fb can be occupied by a residue that is in helix (H), β-strand (E), or coil (C)

Structurally equivalent positions with similar environments were reasoned to have similar structural roles in each BPI domain. Of the 164 structurally aligned positions, 51 have identical 3D-1D environments, corresponding to 31% of the structurally equivalent positions in the BPI domain alignment. This level of conservation is highly significant relative to an alignment of two independently generated random profiles of this length.

The % residue identity expected from two independently generated random sequences of 164 residues was calculated. The expected frequency of matches or % identity is given by:

$$⟨ P ⟩ = {\sum }_{i = 1}^{m} {p}_{i}^{2}$$

where pᵢ is the probability of observing a residue of type i at a given position in a random sequence and m is the number of residue types, which in the case of a sequence alignment is 20 amino acid residues. The standard deviation, σₚ, in the frequency of matches is:

σₚ=\sqrt{P(1−P)/N}

where P is given above and N is the length of the alignment. Probabilities for each amino acid residue, as well as each environment class, were derived from the DAPS database.

The Z-score is given by:

Z=(P_{obs}−<P>)/σₚ

where P_{obs} is the observed % identity for either residues or environment classes between N-terminal and C-terminal domains of BPI. <P> and σₚ are both given above. The Z-score for the sequence identity of the N-terminal and C-terminal domains in BPI is 3.6; higher than expected for independently generated random sequences. A Z-score of 12.9 was calculated for the corresponding conservation of the 3D-1D environments in the 164 structurally aligned positions in BPI. Again, this level of conservation is higher than expected for the alignment of two independently generated random profiles of this length.

The next analyses involved environmentally conserved positions in the BPI alignment as a function of environmental class. To further compare and contrast structural properties between N-terminal and C-terminal domains, three statistical properties were computed for each environment class based upon the structural alignment of BPI. For each environment class, the statistical significance of observing pairs of positions (brought together by the alignment) both belonging to a given environment class was assessed by computing a p-value. The p-value for a given environment class is the probability that one would observe at least as many matches for that class in a random alignment where M is the number of observed matches of that type in the BPI domain structural alignment. The p-value is given by the following equation:

$$p = {\sum }_{( {X}_{12} = M )}^{Min ( {X}_{1} , {X}_{2} )} \frac{( N - {X}_{1} ) ! ( N - {X}_{2} ) ! {X}_{1} ! {X}_{2} !}{( {X}_{1} - {X}_{12} ) ! ( {X}_{2} - {X}_{12} ) ( N - {X}_{1} - {X}_{2} + {X}_{12} ) ! {X}_{12} ! N !}$$

where X₁ is the number of observations for a given environment class from the N-terminal domain, X₂ is the number of observations for the same environment class for the C-terminal domain, and N is the total number of pairs in the alignment. Gaps are not considered. Several of the environment classes have low p-values, and therefore when they are paired in the domain alignment it is of statistical significance, and indicate correlation between the environments of structurally aligned positions. P-values for each environment class are shown in Table 8.

TABLE 8
Log-Odds And Fractional Weighted Log-Odds (FWLO)
Values For 3D-1D Environmental Classes (Env. Class)
For The Alignment Of The N And C-Terminal Domains
Of BPI And For All Alignments From
The DAPS Database

| BPI | | | | DAPS | |
|---|---|---|---|---|---|
| Env. | | | | database | |
| Class | P-value | Log-odds | FWLO | Log-odds | FWLO |
| H:E | 5 × 10⁻² | 1.7 | 0.05 | 1.4 | 0.03 |
| H:P2 | 2 × 10⁻⁸ | 2.0 | 0.24 | 1.2 | 0.11 |
| H:P1 | NO | NO | NO | 1.0 | 0.06 |
| H:B3 | NO | NO | NO | 1.0 | 0.02 |
| H:B2 | NO | NO | NO | 1.0 | 0.02 |
| H:B1 | 3 × 10⁻⁶ | 3.1 | 0.17 | 1.5 | 0.16 |
| E:E | 2 × 10⁻² | 1.4 | 0.06 | 2.1 | 0.01 |
| E:P2 | 1 × 10⁻⁵ | 1.0 | 0.20 | 1.9 | 0.05 |
| E:P1 | 1 × 10⁻¹ | 0.9 | 0.04 | 1.3 | 0.04 |
| E:B3 | NO | NO | NO | 1.5 | 0.03 |
| E:B2 | NO | NO | NO | 1.3 | 0.03 |
| E:B1 | 8 × 10⁻⁶ | 1.3 | 0.19 | 1.7 | 0.23 |
| C:E | 1 × 10⁻¹ | 1.1 | 0.03 | 1.4 | 0.07 |
| C:P2 | 2 × 10⁻¹ | 0.9 | 0.02 | 0.9 | 0.06 |
| C:P1 | NO | NO | NO | 0.9 | 0.03 |
| C:B3 | NO | NO | NO | 1.0 | 0.02 |
| C:B2 | NO | NO | NO | 1.1 | 0.01 |
| C:B1 | NO | NO | NO | 1.3 | 0.02 |

Residues important for stabilizing the cores of proteins tend to be hydrophobic and buried in apolar environments, that is, in the B1 environment class (Table 7). A low p-value for B1-B1 pairs was expected because the structural role of residues that pack in the protein core tend to be conserved. The p-values for the H:B1 and E:B1 environment classes are 3×10⁻⁶ and 8×10⁻⁶, respectively. In other words, observation of at least as many H:B1 matches for the BPI domains in a random alignment is expected only three out of one million times. Thus, positions that belong to the H:B1 or E:B1 classes are observed more often than expected at random. Therefore these positions have conserved structural roles (Table 9).

Positions which belong to the P2 environment class tend to be solvent exposed at the protein's surface. We observed low p-values for the P2 class. The p-values for the H:P2 and E:P2 environment classes are 2×10⁻⁵ and 1×10⁻⁵, respectively, which are as low as the values shown above for the H:B1 and E:B1 classes. While the structural conservation of residues in the protein core is well documented, the conservation of residues on the surface of proteins is not as well described.

Second, the log-odds value for each environment class was calculated. The log-odds ratio for a given pair of environment classes is a measure of how likely it is that any given instance of an environment class is conserved in an alignment. Scores well above zero imply significance, whereas scores near zero imply that the observation is likely to occur when environments are paired randomly. The log-odds value, LO(e) is given by:

$$LO ( e ) = log \frac{⌈ {P}_{AB} ( e ) ⌉}{{P}_{A} ( e ) {P}_{B} ( e )}$$

where P_{AB}(e) is the joint probability of observing environment class e at aligned positions in both sequence A (N-terminal domain) and B (C-terminal domain). P_{A}(e) is the probability of observing the environment class in sequence A, while P_{B}(e) is the probability of observing the same class in sequence B. Results of the analysis are shown in Table 8.

Lastly, the fractional weighted log-odds value (weighted by the joint probability), FWLO(e), was calculated for each environment class. This value is expressed as a fraction of the total weighted log-odds scores for all environment classes. The FWLO(e) value is given by:

$$FWLO ( e ) = \frac{⌈ LO ( e ) \times {P}_{AB} ( e ) ⌉}{{\sum }_{env classes} LO ( e ) \times {P}_{AB} ( e )}$$

a given pair may have a positive LO(e), but if it occurs only rarely in the structural alignment, the FWLO(e) value will be low. Table 9 lists the LO(e) and FWLO(e) values for all identical environment pairs for both the N-terminal and C-terminal domains of BPI.

The H:P2, H:B1, E:P2 and E:B1 environment classes dominate the distribution of LO(e) and FWLO(e) values for each identical environment pair. Large LO(e) values are observed for other environment classes, such as H:E or E:E, but these are relatively rare in BPI and thus unlikely to be as important. This is reflected in the low FWLO(e) values for the H:E and E:E pairs. High FWLO(e) values are observed for the H:P2, H:B1, E:P2 and E:B1 environment classes and are expected play a dominant structural role in the BPI domain.

To examine whether the H:P2, H:B1, E:P2 and E:B1 environment classes had structural significance to proteins other than BPI, we analyzed proteins in the database of aligned protein structures (DAPS) [D. Rice & D. Eisenberg, J. Mol. Biol., 267:1026 (1997)]. DAPS contains 1074 structurally-derived sequence alignments between structurally homologous proteins with less than 25% sequence identity.

LO(e) and FWLO(e) values were calculated over all protein pairs in DAPS (Table 8). The H:P2, H:Bl and E:B1 residue environments had large LO(e) and FWLO(e) values. However, the FWLO(e) value for the E:P2 class is only slightly higher than values for several of the other environment classes. The importance of positions belonging the E:P2 environment class is not general and may be unique to the BPI domain.

For the next analyses, the structural roles of 3D-1D environmentally conserved positions with dissimilar residues in the BPI domain alignment were examined. Thirty-one of the 51 3D-1D environmentally conserved pairs from the BPI domain alignment have similar or identical residues, defined by a positive substitution score from the GONNET matrix [S. Benner et al., Prot. Eng. 7:1323-32 (1994)]. However, 20 structurally equivalent positions with conserved 3D-1D environments have dissimilar residues. The structural roles for residues at these 20 positions were characterized and their importance for stabilizing the BPI fold was determined.

The structural roles for 3D-1D environmentally conserved positions with dissimilar residues were compared by analyzing the tertiary interactions they form with other residues. Favorable contacts were analyzed, including hydrogen bonds, salt bridges, disulfide bonds, van der Waals interactions, or aromatic ring stacking. The similarity in the structural roles was determined for each pair in the alignment.

The structural roles for these pairs fall into three major categories: (i) conserved structuralroles; (ii) auxiliary structural roles; and (iii) different structural roles.

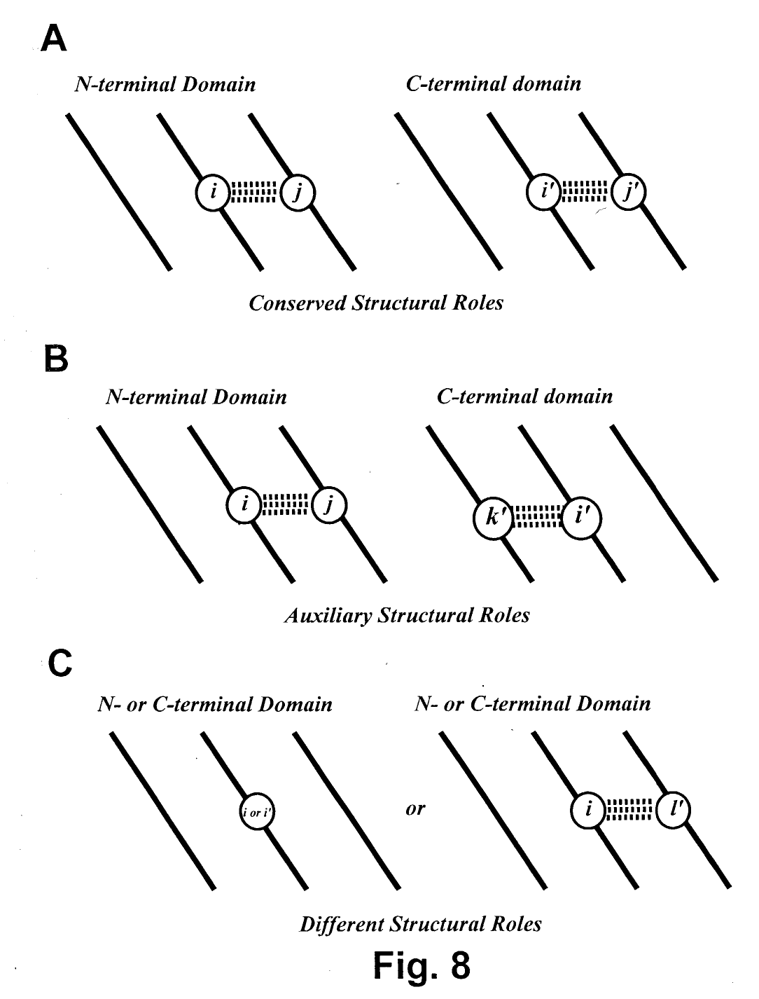

The structural roles for a pair of dissimilar residues are defined as conserved when a residue at position i in the N-terminal domain of BPI forms a tertiary interaction with residue j in the N-terminal domain, and residue i′ in the C-terminal domain forms a tertiary interaction with residue j′. Both residues i and i′ and j and j′ must be at structurally equivalent positions in the alignment for the structural roles to be defined as conserved. The type of tertiary interaction does not have to be the same (FIG. 8(a)).

In auxiliary structural roles, residue i forms a tertiary interaction with residue j in the N-terminal domain, and residue i′ in the C-terminal domain forms a tertiary interaction with residue k′. Residues i and i′ must occupy structurally equivalent positions, but residues j and k′ do not have to be aligned (FIG. 8(b)).

For different structural roles, residues i and i′ belong to the same environment class but one or both of the residues have no corresponding structural role, or one or both residues are involved in stabilizing the interface between the domains in the BPI structure rather than the domain itself (FIG. 8(c)). For example, either residue i or i′ is solvent exposed, or residue i from the N-terminal domain interacts with residue l′ from the C-terminal domain. A summary of the structural roles for the 20 pairs of dissimilar residues is given in Table 9.

TABLE 9
Classification Of The Structural Roles For Equivalent Positions
With Conserved 3D-1D Environment Classes And Dissimilar Residue
Types. Comparison Of Structural Roles For The N-Terminal (N)
And C-Terminal (C) Domains Of BPI

| Residue | N-terminal domain | | | C-terminal domain | |
|---|---|---|---|---|---|
| (n) | Atom | Structural role | Residue (c) | Atom | Structural role |

A. Conserved structural roles

| | | | | | |
|---|---|---|---|---|---|
| Ser79 | OG | H bond to Ser69 OG through | Tyr336 | OH | H bond to Ser327 OG |
| | | a water molecule | | | |
| Ser112 | OG | H bond to bb carbonyl group | His361 | CE1 | VdW with Leu385 CB and |
| | | of His138 through a water | | | CD1 |
| | | molecule | | | |
| Asp116 | OD2 | H bond to Ser134 OG | Ser365 | OG | H bond to Lys380 NZ |
| Thr219 | OG1 | H bond to Thr216 OG1 | Phe446 | — | Ring stacking with His443 |

B. Auxiliary structural roles

| | | | | | |
|---|---|---|---|---|---|
| Tyr16 | OH | H bond to Gln20 NH2 | Thr265 | OG1 | H bond to bb carbonyl group |
| | | | | | of Phe262 |
| Glu19 | NE2 | H bond to Gln20 OE1 | Leu268 | CG, CD1 | VdW with Glu272 CD and |
| | | | | | CG |
| Asp36 | OD1 | H bond to bb amide of Ser55 | Thr294 | OG1 | H bond to bb amide group of |
| | | | | | Met312 |
| Lys77 | NZ | Salt bridge to Asp116 OD1 | Thr334 | OG1 | H bond to Gln329 NE2 |
| Ala83 | CB | VdW with Leu63 CD2 and | Val339 | CG1 | VdW with Met360 CB |
| | | Ile 80 CG2 | | | |
| Asn180 | ND2 | H bond to Ser184 OG | Pro415 | CB, CD | VdW with Arg416 CZ and |
| | | | | | Leu414 CB |
| Ser184 | OG | H bond to Asn 180 ND2 | Glu419 | OE2 | Salt bridge to Arg416 NH2 |
| Pro188 | — | Solvent exposed; | Lys423 | — | Solvent exposed |
| Asn206 | CG | VdW with Lys225 CB and | Val433 | CG1, CG2 | VdW with Val453 CG1 |
| | | CG | | | |
| Val222 | CG1 | VdW with Leu209 CG and | Phe449 | CD1, CE1, CE2 | VdW with Leu440 CB and |
| | | CD1 | | | CD1 and Val368 CG1 |
| Glu227 | OE1 | Salt bridge to Lys225 NZ | Val454 | CB | VdW with Tyr436 CE1 |

C. Different Structural Roles

| | | | | | |
|---|---|---|---|---|---|
| Val1 | — | Solvent exposed | Asp251 | OD1 | H bond with Arg252 NH1 |
| | | | | | and Tyr255 OH |
| Lys12 | NZ | Salt bridge with Asp452 OD1 | Tyr261 | CG, CD1, | VdW w/Pro241 CG, CD |
| | | | | CD2, CE2, | |
| | | | | CZ | |
| Gly120 | — | Solvent exposed; | Ser369 | OG | H bond to bb carbonyl group |
| | | | | | of Ala370 |
| His138 | — | Solvent exposed | Leu385 | CB, CD1 | VdW with His361 CE1 |
| Thr215 | OG | H bond to ordered water | Pro442 | CB, CG | VdW with Leu447 CD1. |

H bond, Hydrogen-bond interaction;
VdW, a van der Waals interaction;
backbone, bb

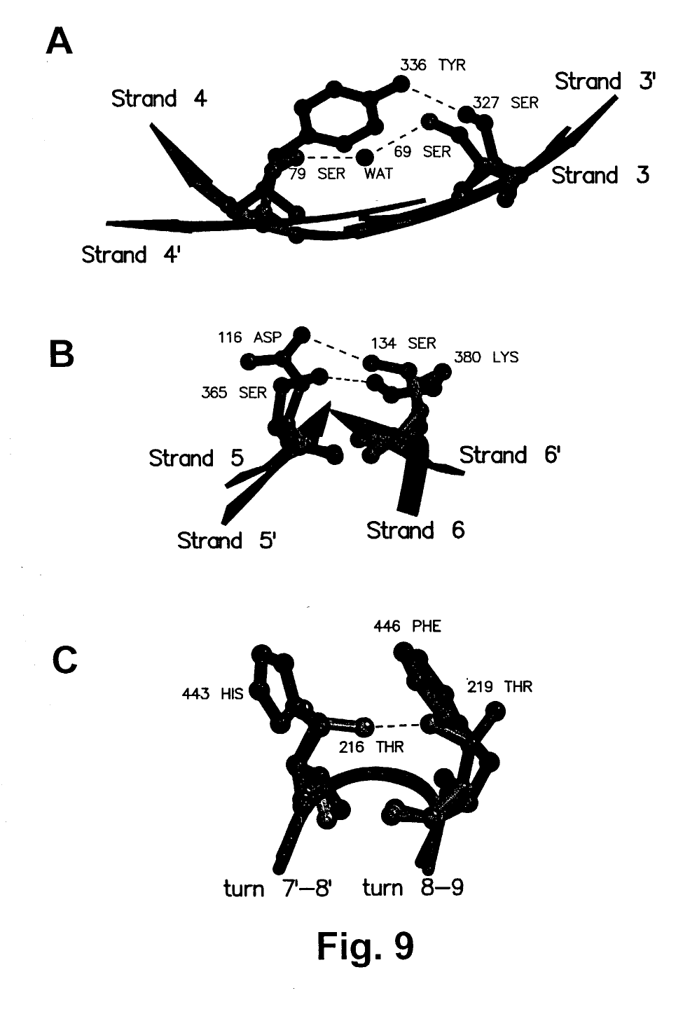

Four pairs of 3D-1D environmentally conserved positions with dissimilar residue types fall into the conserved structural roles category. FIG. 9 shows examples of pairs of 3D-1D environmentally conserved residues with dissimilar residues yet conserved structural roles from the BPI domain alignment. Residues and secondary structure elements from the N-terminal domain are blue; residues and secondary structure elements from the C-terminal are red. The relationship of the secondary structure elements to the entire BPI molecule is shown in FIG. 7(c). Hydrogen bonds are shown as broken lines between the hydrogen bond donor and acceptor atoms. For example, Ser79 and Tyr336 both belong to the E:P2 environment class, however the substitution of serine for tyrosine in the GONNET matrix [S. Benner et al., Prot. Eng. 7:1323 (1994)] is not favorable. The conserved structural roles of both Ser79 and Tyr336 are to stabilize the domain by connecting adjacent β-strands. These residues are located on β-strands 4 and 4′ respectively (see FIGS. 7(c) and 9(al )). Ser79 (N-terminal domain) forms a 2.7 Å hydrogen bond to a water molecule which is hydrogen bonded to Ser69, located on β-strand 3. The crystallographic temperature factor for this water molecule is 27 Å² so it is well ordered. In the C-terminal domain, Tyr336 forms a 2.8 Å hydrogen bond to Ser327, which lies on β-strand 3′. Ser69 and Ser327 are also paired in the BPI domain alignment. Therefore, Ser79 and Tyr336 interact with structurally equivalent residues in their respective domains, conserving the structural role for these dissimilar residues.

A similar example involves Asp116 and Ser365, located on β-strands 5 and 5′, respectively (FIG. 9(b)). The conserved structural roles of Asp116 and Ser365 connect adjacent β-strands in their respective domains. Asp116 forms a 2.9 Å hydrogen bond to Ser134 on β-strand 6. Ser365 forms a 3.1 Å hydrogen bond to Lys380 O on β-strand 6′. Ser134 and Lys380 are paired in the BPI domain alignment.

Conserved structural roles do not necessarily involve identical tertiary interactions. For example, Thr219 and Phe446 are structurally equivalent residues with identical environment classes (E:P2). Thr219 forms a 2.7 Å hydrogen bond with Thr216 which stabilizes a tight turn from β-strand 8 to β-strand 9 (FIG. 9(c)). In the C-terminal domain, the aromatic ring of Phe446 is approximately 4 Å from the ring of His443. Ring stacking of phenylalanine and histidine residues has been shown to be an energetically favorable interaction [J. Mitchell et al., J. Mol. Biol., 239:315 (1994)]. Therefore, the interaction of Phe446 with His443 stabilizes a tight turn from β-strand 7′ to β-strand 8′, similar to the interaction found in the N-terminal domain.

Eleven 3D-1D environmentally conserved positions with dissimilar residue types have auxiliary structural roles in the BPI domains. The structural roles of these residue pairs are auxiliary because they help stabilize the fold, even though the structural roles are not strictly conserved. An example is Lys77 and Thr334, located in β-strand 4 and 4′ respectively (FIG. 7(c)). Lys77 forms a salt bridge with Asp116 located on β-strand 5. Thr334 forms a hydrogen bond to Gln329 located on β-strand 3′. These interactions still support the fold of each BPI domain by connecting adjacent β-strands.

Five of the 3D-1D environmentally conserved positions with dissimilar residue types have different structural roles in the two domains. Some of these positions, such as His138, are completely exposed to bulk solvent and make no contacts with any ordered atoms in the BPI structure, whereas the structurally equivalent residue from the C-terminal domain, Leu385, makes van der Waals contacts with His361.

Another example of a pair of residues with different structural roles is Lys12 and Tyr261. Lys12 forms a salt bridge with Asp452, connecting the N-terminal domain of BPI with the C-terminal domain. Tyr261 forms van der Waals contacts with Pro241, located in the domain linker. The structural roles for these two residues are different because Lys12 stabilizes the interface between the two domains and Tyr261 interacts with the domain linker.

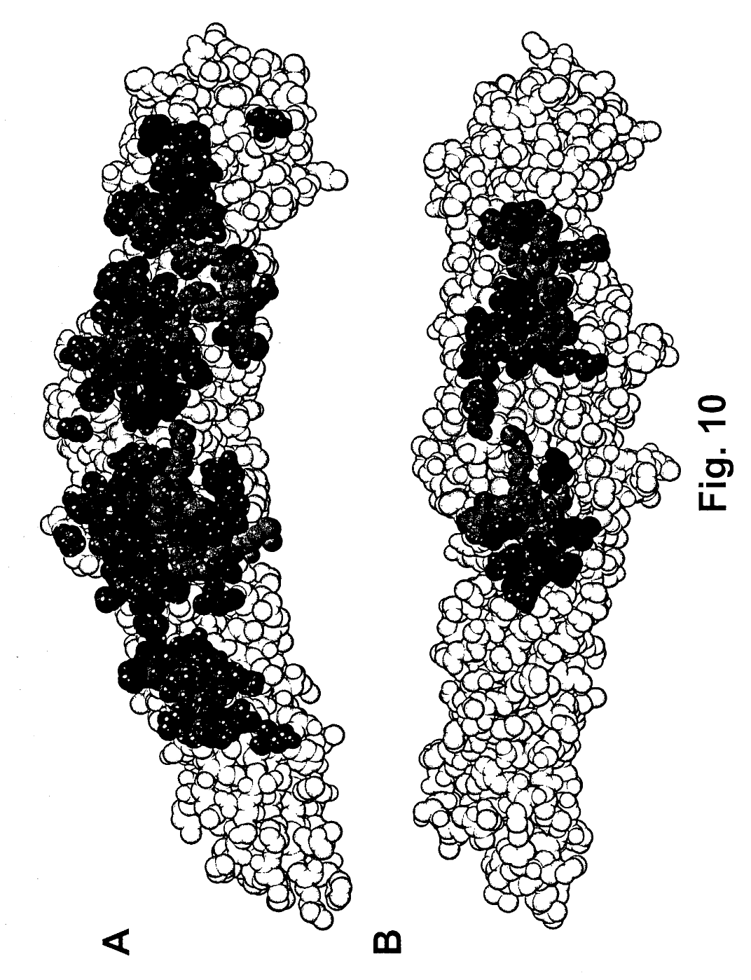

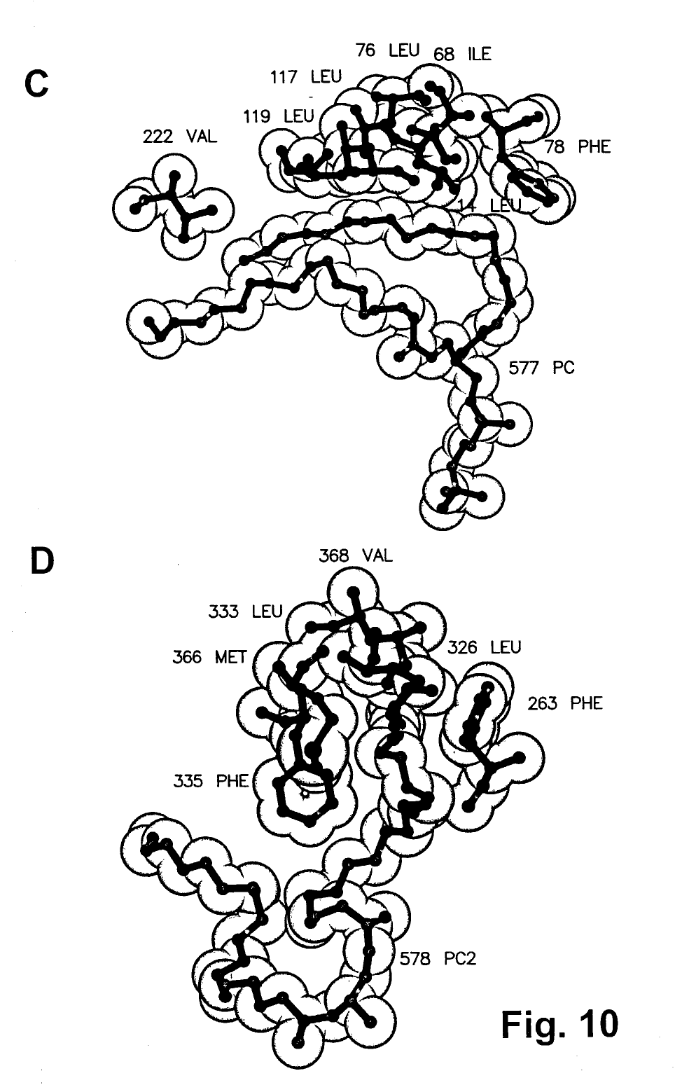

For the next analyses, the clustering of 3D-1D environmentally conserved positions in both the BPI fold and the lipid binding pockets was studied. FIG. 10(a) shows a space-filling representation of BPI. 3D-1D environmentally conserved positions are colored red. The two bound phospholipids are gold. The N-terminal domain is shown on the left. FIG. 10(b) shows a spaced-filling representation of BPI rotated 90° about the x-axis relative to FIG. 10(a). Positions colored dark blue: (1) have conserved 3D-1D environments and are paired in the structural alignment, (2) contain at least one atom in contact with at least one lipid atom; (3) both positions are in contact with at least one lipid atom. Positions that are colored cyan: (1) and (2) hold but not (3). Positions that are colored green: 3D-1D environmentally conserved positions from the C-terminal domain that are in contact with the lipid-binding pocket of the N-terminal domain. Positions colored purple: 3D-1D-conserved positions from the N-terminal domain that are in contact with the lipid-binding pocket of the C-terminal domain. FIG. 10(c) shows the interaction of 3D-1D environmentally conserved positions in the N-terminal lipid-binding pocket. These seven positions satisfy the following constraints: (1) have conserved 3D-1D environments and are paired in the structural alignment; (2) contain at least one atom in contact with at least one lipid atom; (3) structurally equivalent positions in the C-terminal domain are also in contact with at least one lipid atom. The residues occupying these positions are also shown in 10(b) as blue spheres. The van der Waals radii are also shown for the atoms. These residues form a cluster which interacts exclusively with only one acyl chain of the. phospholipid, shown as gray ball-and-sticks. FIG. 10(d) shows the interaction of 3D-1D environmentally conserved positions in the C-terminal lipid-binding pocket. These six positions also satisfy the same constraints as the equivalent N-terminal positions. The residues interact with only one acyl chain of the lipid.

Positions with conserved 3D-1D enviromnents tend to cluster in the BPI fold. The location of the 51 3D-1D environmentally conserved pairs are highlighted in FIG. 10(a). These positions tend to cluster around the core of each domain and the phospholipid binding pockets, while the two tips of the molecule contain very few 3D-1D environmentally conserved residues. Clustered positions predominantly belong to classes with mostly buried and apolar environments (H:B1 or E:B1) or mostly solvent accessible environments (H:P2, or E:P2).

The degree of clustering was assessed by calculating C^{∝}-C^{∝} distances for all 3D-1D environmentally conserved positions in each domain. For this analysis, a position is considered a tertiary neighbor of another position if its C^{∝} atom is less than 7 Å away but is not within two residues on the peptide chain. 3D-1D environmentally conserved positions were found to have at least one other conserved tertiary neighbor 41 and 44% of the time for the N-terminal and C-terminal domains, respectively. In contrast, positions that were not 3D-1D environmentally conserved have tertiary neighbors that were conserved only 24 and 28%, confirming that 3D-1D environmentally conserved positions tend to cluster.

3D-1D environmentally conserved positions in the lipid-binding pockets of each domain also cluster. The program CAST [J. Liang, et al., Prot. Sci., 7:1884-1897 (1998)] was used to identify all positions in contact with at least one lipid atom. Fifty-one positions contribute to the N-terminal lipid-binding pocket and 43 positions contribute to the C-terminal pocket. A total of 11 of these positions from the N-terminal pocket are 3D-1D environmentally conserved; 15 positions from the C-terminal pocket are 3D-1D environmentally conserved. While the number of 3D-1D environmentally conserved positions between the domains of BPI must be equal, the number of conserved positions in the lipid-binding pockets can differ. This is explained by the observation that some positions located in a lipid-binding pocket are aligned to positions that are not located in the other pocket. Therefore, only eight of the environmentally conserved positions in each pocket are paired in the BPI domain alignment.

The eight structurally equivalent positions from the lipid-binding pockets cluster in each domain (FIG. 10(b)). Seven of the eight residues at these positions in the N-terminal lipid-binding pocket interact exclusively with only one acyl chain of the phospholipid (FIG. 10(c)). Six of the eight residues at these positions in the C-terminal pocket interact exclusively with only one acyl chain of the lipid (FIG. 10(d)).

Nearly all of the 3D-1D environmentally conserved positions in the lipid-binding pockets belong to either the H:B1 (helical secondary structure and buried) or E:B1 (□-strand secondary structure and buried) environment classes. This is as one would expect because buried, hydrophobic residues contribute to pockets which bind apolar molecules. An exception occurs at the Phe228-Tyr455 pair, where the conserved environment class is E:P2. These residues are near the mouth of the lipid-binding pockets and are exposed to solvent.

The structure coordinates described in Example 4 and listed in Table 6 and the analyses described in this Example are useful for the designing and making of new and useful products based on BPI, including a BPI protein, including a fragment, analog or variant thereof, or of a BPI related lipid transfer protein, including a fragment, analog or variant thereof.

What is claimed is:

1. Use of atomic coordinates of bactericidal/permeability-increasing (“BPI”) protein, or fragment, analog or variant thereof, to model a BPI protein.

2. Use of atomic coordinates of bactericidal/permeability-increasing (“BPI”) protein, or fragment, analog or variant thereof, to model a BPI-related lipid transfer protein.

3. The use according to claim 2, wherein the BPI-related lipid transfer protein is lipopolysaccharide-binding protein (LBP), cholesteryl ester transferase protein (CETP) or phospholipid transfer protein (PLTP), or fragment, analog or variant thereof.

4. The use according to any of claims 1-3, wherein the BPI protein comprises a binding site characterized by amino acid residues of at least one binding pocket as defined in Table 3.

5. The use according to any of claims 1-3, wherein the BPI protein comprises a binding site characterized by at least one amino acid sequence, or variant of the sequence, selected from positions about 17 to about 45, positions about 36 to about 54, positions about 65 to about 99, positions about 84 to about 109, positions about 142 to about 164, or positions about 142 to about 169 of BPI.

6. The use according to any of claims 1-3, wherein the BPI protein comprises a binding site characterized by amino acid residues of at least one binding pocket as defined in Table 3 and a binding site characterized by at least one amino acid sequence, or variant of the sequence, selected from positions about 17 to about 45, positions about 36 to about 54, positions about 65 to about 99, positions about 84 to about 109, positions about 142 to about 164, or positions about 142 to about 169 of BPI.

7. Use of atomic coordinates of bactericidal/permeability-increasing (“BPI”) protein to computationally design a chemical compound for mimicking BPI protein, or fragment, analog or variant thereof.

8. Use of atomic coordinates of bactericidal/permeability-increasing (“BPI”) protein to computationally design a chemical compound for mimicking a BPI-related lipid transfer protein, or fragment, analog or variant thereof.

9. The use according to claim 8, wherein the BPI-related lipid transfer protein is lipopolysaccharide-binding protein (LBP), cholesteryl ester transferase protein (CETP) or phospholipid transfer protein (PLTP).

10. Use of atomic coordinates of bactericidal/permeability-increasing (“BPI”) protein, to design a chemical compound capable of associating with a BPI-related lipid binding protein, or fragment, analog or variant thereof.

11. The use according to claim 10, wherein the BPI-related lipid binding protein is lipopolysaccharide-binding protein (LBP), cholesteryl ester transferase protein (CETP) or phospholipid transfer protein (PLTP), or fragment, analog or variant thereof.

12. Use of atomic coordinates of bactericidal/permeability-increasing (“BPI”) protein to design a model of ligands in an active site of a lipid binding protein.

13. The use according to claim 12, wherein the lipid binding protein is bactericidal/permeability-increasing protein (BPI), lipopolysaccharide-binding protein (LBP), cholesteryl ester transferase protein (CETP) or phospholipid transfer protein (PLTP), or fragment, analog or variant thereof.

14. Use of atomic coordinates of bactericidal/permeability-increasing (“BPI”) protein, to design compounds with at least one activity of antibacterial, antifungal, antimycobacterial, antichlamydial, antiprotozoan, heparin-binding, endotoxin-binding, heparin-neutralizing, endotoxin-neutralizing, inhibition of tumor or endothelial cell proliferation, inhibition of angiogenesis, anti-inflammatory, anticoagulant, antithrombolytic, enhancement of pericyte cell proliferation, enhancement of antibiotic activity or susceptibility, or inhibition of H⁺/K⁺ ATPase activity.

15. The use according to any of claims 1-14, wherein atomic coordinates are according to Table 4.

16. The use according to any of claims 1-14, wherein the atomic coordinates are according to Table 6.

17. A method of three-dimensional modeling of a bactericidal/permeability-increasing (“BPI”) protein comprising the steps of:
(a) providing three-dimensional atomic coordinates derived from X-ray diffraction measurements of a BPI protein in a computer readable format;
(b) inputting the data from step (a) into a computer with appropriate software programs;
(c) generating a three-dimensional structural representation of the BPI protein suitable for visualization and further computational manipulation.

18. A method of three-dimensional modeling of a bactericidal/permeability-increasing (“BPI”)-related lipid transfer protein comprising the steps of:
(a) providing three-dimensional atomic coordinates derived from X-ray diffraction measurements of a BPI protein in a computer readable format;
(b) inputting the data from step (a) into a computer with appropriate software programs;
(c) generating a three-dimensional structural representation of the BPI-related lipid transfer protein suitable for visualization and further computational manipulation.

19. The use according to any of claims 17-18, wherein the BPI protein comprises a binding site characterized by amino acid residues of at least one binding pocket as defined in Table 3.

20. The use according to any of claims 17-18, wherein the BPI protein comprises a binding site characterized by at least one amino acid sequence, or variant of the sequence, selected from positions about 17 to about 45, positions about 36 to about 54, positions about 65 to about 99, positions about 84 to about 109, positions about 142 to about 164, or positions about 142 to about 169 of BPI.

21. The use according to any of claims 17-18, wherein the BPI protein comprises a binding site characterized by amino acid residues of at least one binding pocket as defined in Table 3 and a binding site characterized by at least one amino acid sequence, or variant of the sequence, selected from positions about 17 to about 45, positions about 36 to about 54, positions about 65 to about 99, positions about 84 to about 109, positions about 142 to about 164, or positions about 142 to about 169 of BPI.

22. A method for providing an atomic model of a BPI protein, or fragment, analog or variant thereof, comprising
(a) providing a computer readable medium having stored thereon atomic coordinate/x-ray diffraction data of the BPI protein, or fragment, analog or variant thereof, in crystalline form, the data sufficient to model the three-dimensional structure of the BPI protein, or fragment, analog or variant thereof;
(b) analyzing, on a computer using at least one subroutine executed in the computer, atomic coordinate/x-ray diffraction data from (a) to provide atomic coordinate data output defining an atomic model of the BPI protein, or fragment, analog or variant thereof, said analyzing utilizing at least one computing algorithm selected from the group consisting of data processing and reduction, auto-indexing, intensity scaling, intensity merging, amplitude conversion, truncation, molecular replacement, molecular alignment, molecular refinement, electron density map calculation, electron density modification, electron map visualization, model building, rigid body refinement, positional refinement; and
(c) obtaining atomic coordinate data defining the three-dimensional structure of at least one of the BPI protein, or fragment, analog or variant thereof.

23. A method according to claim 22, wherein the computer readable medium further has stored thereon data corresponding to a nucleic acid sequence or an amino acid sequence data comprising at least one structural domain or functional domain of a BPI, LBP, CETP or PLTP corresponding to at least one BPI or mutant primary sequence of FIG. 5 or Table 2, or a fragment thereof; and wherein said analyzing step further comprises analyzing the sequence data.

24. A computer-based system for providing atomic model data of the three-dimensional structure of BPI protein, or fragment, analog or variant thereof, a BPI mutant or a BPI fragment, comprising the following elements:
(a) at least one computer readable medium (CRM) having stored thereon atomic coordinate/x-ray diffraction data of the BPI protein, or fragment, analog or variant thereof;
(b) at least one computing subroutine that, when executed in a computer, causes the computer to analyze atomic coordinate/x-ray diffraction data from (a) to provide atomic coordinate data output defining an atomic model of the BPI protein, or fragment, analog or variant thereof, said analyzing utilizing at least one computing subroutine selected from the group consisting of data processing and reduction, auto-indexing, intensity scaling, intensity merging, amplitude conversion, truncation, molecular replacement, molecular alignment, molecular refinement, electron density map calculation, electron density modification, electron map visualization, model building, rigid body refinement, positional refinement; and
(c) retrieval means for obtaining atomic coordinate output data substantially defining the three-dimensional structure of the BPI protein, or fragment, analog or variant thereof.

25. A method for providing a computer atomic model of a ligand of a BPI protein, or fragment, analog or variant thereof, comprising
(a) providing a computer readable medium (CRM) having stored thereon atomic coordinate data of a BPI protein, or fragment, analog, or variant thereof;
(b) providing a CRM having stored thereon atomic coordinate data sufficient to generate atomic models of potential ligands of the BPI protein, or fragment, analog, or variant thereof;
(c) analyzing on a computer, using at least one subroutine executed in the computer, the atomic coordinate data from (a) and ligand data from (b), to determine binding sites of BPI protein, or fragment, analog, or variant thereof, and to provide atomic coordinate data defining an atomic model of at least one ligand of the BPI, BPI mutant or a fragment thereof, said analyzing utilizing computing subroutines selected from the group consisting of data processing and reduction, auto-indexing, intensity scaling, intensity merging, amplitude conversion, truncation, molecular replacement, molecular alignment, molecular refinement, electron density map calculation, electron density modification, electron map visualization, model building, rigid body refinement, positional refinement; and
(d) obtaining atomic coordinate model output data defining the three-dimensional structure of said at least one ligand of the BPI protein, or fragment, analog, or variant thereof.

26. A computer-based system for providing an atomic model of at least one ligand of a BPI, BPI mutant or a fragment thereof, comprising the following elements;
(a) a computer readable medium (CRM) having stored thereon atomic coordinate data of a BPI, mutant or fragment thereof;
(b) a CRM having stored thereon atomic coordinate data sufficient to generate atomic models of potential ligands of a BPI, mutant or fragment;
(c) at least one computing subroutine for analyzing on a computer, the atomic coordinate data from (a) and (b), to determine binding sites of BPI protein, or fragment, analog, or variant thereof, and to provide data output defining an atomic model of at least one potential ligand of BPI protein, or fragment, analog, or variant thereof, said analyzing utilizing at least one computing subroutine selected from the group consisting of data processing and reduction, auto-indexing, intensity scaling, intensity merging, amplitude conversion, truncation, molecular replacement, molecular alignment, molecular refinement, electron density map calculation, electron density modification, electron map visualization, model building, rigid body refinement, positional refinement; and
(d) retrieval means for obtaining atomic coordinate data of said at least one ligand of a BPI protein, or fragment, analog or variant thereof.
